(19) United States
(12) Patent Application Publication
(10) Pub. No.: US 2003/0113798 A1
(43) Pub. Date: Jun. 19, 2003

(54) Antigenic peptides, such as for G protein-coupled receptors (GPCRS), antibodies thereto, and systems for identifying such antigenic peptides

(76) Inventors: Glenna C. Burmer, Seattle, WA (US); Christine L. Roush, Seattle, WA (US); Joseph P. Brown, Seattle, WA (US)

Correspondence Address: Joshua King, GRAYBEAL JACKSON HALEY LLP, Suite 350, 155-108th Avenue N.E., Bellevue, WA, 98004-5973, US

(21) Appl. No.: 10/225,567

(22) Filed: Aug. 19, 2002

(30) Foreign Application Priority Data
Dec. 19, 2001 (WO) PCT/US01/50107

(51) Int. Cl. G01N 33/53; C07K 14/705; C07K 16/28; G01N 33/537; G01N 33/543

(52) U.S. Cl. 435/7.1; 530/388.22; 530/350; 435/7.92; 435/7.93

(57) ABSTRACT

The present invention provides antigenic peptides for GPCRs and antibodies relating thereto, and related systems, methods, compositions, and the like, such as diagnostics and medicaments. Where antibodies against a given GPCR are not known, the present invention provides such antibodies, and preferred antigenic sequences for producing such antibodies. Where antibodies against a given GPCR are known, the present invention provides preferred antigenic peptides for producing antibodies that exhibit improved specificity, affinity or capacity to perform antibody-related actions relative to the known antibodies.

### CROSS-REFERENCE TO RELATED APPLICATIONS

The present application claims priority from U.S. provisional patent application No. 60/257,144, filed Dec. 19, 2000 and presently pending.

### TABLE OF CONTENTS

The following is a Table of Contents to assist review of the present application:

CROSS-REFERENCE TO RELATED APPLICATIONS

TABLE OF CONTENTS

BACKGROUND

SUMMARY

BRIEF DESCRIPTION OF THE DRAWING

DETAILED DESCRIPTION

A. INTRODUCTION AND OVERVIEW

B. DEFINITIONS

C. SELECTION OF DESIRED ANTIGENIC PEPTIDES FOR GPCRs AND OTHER POLYPEPTIDES

D. GENERAL DISCUSSION OF ANTIGENIC PEPTIDES RELATED TO PARTICULAR GPCRS

ANTIGENIC PEPTIDES GENERALLY:

EXPRESSION PROFILES BASED ON PROTEINS:

SCREENING FOR ACTIVITY:

PROTEIN PURIFICATION:

E. CERTAIN ASSAYS, ANTIBODIES, PROBES, THERAPEUTICS, AND OTHER SYSTEMS AND ASPECTS, OF THE INVENTION

1. SYSTEMS AND METHODS FOR SCREENING FOR A PARTICULAR GPCR OR ANTIGENIC PEPTIDE

SCREENING FOR ANTIGENIC PEPTIDES:

SCREENING FOR/WITH ANTIGENIC PEPTIDES:

LIST OF ASSAYS:

ENZYME-LINKED IMMUNOSORBENT ASSAYS (ELISA):

IMMUNOFLUORESCENCE ASSAY:

BEAD AGGLUTINATION ASSAYS:

ENZYME IMMUNOASSAYS:

SANDWICH ASSAY:

SEQUENTIAL AND SIMULTANEOUS ASSAYS:

IMMUNOSTICK (DIP-STICK) ASSAYS:

IMMUNOCHROMATOGRAPHIC ASSAYS:

IMMUNOFILTRATION ASSAYS:

BIOSENSOR ASSAYS:

2. ANTIBODIES

ANTIBODIES GENERATED AGAINST A PARTICULAR ANTIGENIC PEPTIDE AND ITS CORRESPONDING GPCR:

ANTIBODIES GENERALLY:

ANTI-IDIOTYPIC ANTIBODIES:

a. Antibody Preparation

(i) Polyclonal Antibodies

ANTIBODY PREP—POLYCLONAL:

ANTIBODY PREP—ADJUVANTS (ALL ABS):

(ii) Monoclonal Antibodies

ANTIBODY PREP—MONOCLONAL:

MOABS—COMBINATORIAL:

HUMANIZED MOAB:

ANTIBODY SUBSTITUTIONS—NON-IMMUNOGLOBULIN POLYPEPTIDES (ALL ABS):

CHIMERICS:

ANTIBODY LABELING (ALL ABS):

(iii) Humanized And Human Antibodies

HUMANIZED AB GENERALLY:

(iv) Antibody Fragments

ANTIBODY FRAGMENTS:

(v) Bispecific Antibodies

BISPECIFIC ANTIBODIES GENERALLY:

ANTIBODIES—HYBRID IMMUNOGLOBULIN HEAVY CHAIN:

ANTIBODIES—CROSS-LINKED OR “HETEROCONJUGATE”:

ANTIBODIES—DIABODIES:

ANTIBODIES—OTHER:

b. Antibody Purification

ANTIBODY PURIFICATION GENERALLY:

BEFORE LPHIC:

LPHIC:

POST LPHIC:

c. Some Uses For Antibodies Described Herein

(i) Generally

GENERALLY:

ASSAYS:

DIAGNOSTIC USES:

(ii) Assays

ASSAYS:

COMPETITIVE BINDING ASSAYS:

(iii) Affinity Purification

AFFINITY PURIFICATION:

(iv) Therapeutics

THERAPEUTIC USES:

THERAPEUTIC FORMULATIONS:

THERAPEUTIC FORMULATIONS—STERILE:

THERAPEUTIC ADMINISTRATIONS:

THERAPEUTIC ADMINISTRATIONS—SUSTAINED RELEASE-POLYMERS:

THERAPEUTIC ADMINISTRATIONS—SUSTAINED RELEASE-LIPOSOMES:

THERAPEUTICALLY EFFECTIVE AMOUNT:

5. DRUG DESIGN BASED ON THE ANTIGENS HEREIN OR ANTIBODIES THERETO

DISEASE/CONDITIONS LIST:

EXAMPLES

SEQUENCE LISTING:

CLAIMS

ABSTRACT

### BACKGROUND

G protein-coupled receptors (GPCRs) are a large group of proteins that transmit signals across cell membranes. In general terms, GPCRs function somewhat like doorbells. When a molecule outside the cell contacts the GPCR (pushes the doorbell), the GPCR changes its shape and activates “G proteins” inside the cell (similar to the doorbell causing the bell to ring inside the house, which in turn causes people inside to answer the door). GPCRs are like high-security doorbells because each GPCR responds to only one specific kind of signaling molecule (called its “endogenous ligand”), kind of like a high-tech door lock that responds to only one fingerprint. Part of the GPCR is located outside the cell (the “extracellular domain”), part spans the cell's membrane (the “transmembrane domain”), and part is located inside the cell (the “intracellular domain”). Thus, GPCRs are embedded in the outer membrane of a cell and recognize and bind certain signaling molecules that are present in the spaces surrounding the cell. GPCRs are used by cells to keep an eye on the cells' own activity and on the environment. In organisms that have many cells, the cells use GPCRs to talk to each other.

GPCRs are important to the pharmaceutical industry and other industries. For example, many drugs, including some antibody-based drugs, act by binding to specific GPCRs and initiating or inhibiting their intracellular actions, and diagnostics and therapeutics based on GPCRs or on antibodies for GPCRs are becoming increasingly important.

General concepts about GPCRs are discussed in more scientific terms in the following paragraphs.

The GPCR superfamily has at least 250 members, Strader et al., FASEB J., 9:745-754 (1995); Strader et al., Annu. Rev. Biochem., 63:101-32 (1994). GPCRs play important roles in diverse cellular processes including cell proliferation and differentiation, leukocyte migration in response to inflammation, gene transcription, vision (the rhodopsins), smell (the olfactory receptors), neurotransmission (muscarinic acetylcholine, dopamine, and adrenergic receptors), and hormonal response (luteinizing hormone and thyroid-stimulating hormone receptors). Strader et al., supra; U.S. Pat. Nos. 5,994,097 and 6,063,596. Many important drugs produce their therapeutic actions through their interaction with GPCRs.

Nucleotide and amino acid sequences for many GPCRs have been reported and can be found in public databases such as GenBank and GenPept. Generally speaking, different GPCRs show both structural and sequence similarities. The most conserved domains of GPCRs are the transmembrane domains and the first two cytoplasmic loops. GPCRs range in size from under 400 to over 1000 amino acids. Coughlin, S. R., Curr. Opin. Cell Biol. 6:191-197 (1994). They contain seven hydrophobic transmembrane regions that span the cellular membrane and form a bundle of antiparallel alpha helices. McKee K. K., supra. The bundle of helices forming the transmembrane regions provide many structural and functional features of the receptor. In most cases, the bundle of helices form a pocket that binds a signaling molecule. However, when the binding site accommodates larger molecules, the extracellular N-terminal segment or one or more of the three extracellular loops participate in binding and in subsequent induction of conformational change in the intracellular portions of the receptor. These helices are joined at their ends by three intracellular and three extracellular loops. GPCRs also contain cysteine disulfide bridges between the second and third extracellular loops, an extracellular N-terminus, and a cytoplasmic or intracellular C-terminus. The N-terminus is often glycosylated, while the C-terminus is generally phosphorylated. A conserved, acidic-Arg-aromatic triplet present in the second cytoplasmic loop may interact with G Proteins. Most GPCRs contain a characteristic consensus pattern. Watson, S. and S. Arkinstall, The G protein Linked Receptor Facts Book, Academic Press, San Diego, Calif. (1994); Bolander, F. F. Molecular Endocrinology, Academic Press, San Diego, Calif. (1994).

Although GPCRs have many features in common, each GPCR has its own unique characteristics as well. GPCRs have varying nucleotide and amino acid sequences, and varying antigenicity. GPCRs bind a diverse array of specific, extracellular signaling molecules (which can also be referred to as “ligands”) including peptides, cytokines, hormones, neurotransmitters, growth factors, and specialized stimuli such as photons, flavorants, and odorants. Identified ligands include, for example, purines, nucleotides (e.g., adenosine, cAMP, NTPs), biogenic amines (e.g., epinephrine, norepinepherine, dopamine, histamine, noradrenaline, serotonin), acetylcholine, peptides (e.g., angiotensin, calcitonin, chemokines, corticotropin releasing factor, galanin, growth hormone releasing hormone, gastric inhibitory peptide, glucagon, neuropeptide Y, neurotensin, opioids, thrombin, secretin, somatostatin, thyrotropin releasing hormone, vasopressin, vasoactive intestinal peptide), lipids and lipid-based compounds (e.g., cannabinoids, platelet activating factor), excitatory and inhibitory amino acids (e.g., glutamate, GABA), ions (e.g., calcium), and toxins.

In general, a GPCR binds only one type of signaling molecule and GPCRs are classified according to subfamilies based upon their selectivity and specificity for a particular ligand. When the ligand for a receptor is not known, the receptor is known as an orphan receptor. The extracellular domain interacts with or binds to certain signaling molecules or ligands located outside of the cell. The binding of a ligand to the extracellular domain alters the conformation of the receptor's intracellular domain causing the activation of a G protein. The G protein then activates or inactivates a separate plasma-membrane-bound enzyme or ion channel. This chain of events alters the concentration of one or more intracellular messengers (second messengers) such as cyclic AMP (cAMP), inositol triphosphate, diacylglycerol, or Ca²⁺. These, in turn, alter the activity of other intracellular proteins such as cAMP-dependent protein kinase and Ca²⁺/calmodulin-dependent protein kinases, leading to the transduction and amplification of the original extracellular signal. Baldwin, J. M., Curr. Opin. Cell Biol. 6:180-190 (1994). The G protein is deactivated by hydrolysis of GTP by GTPase. U.S. Pat. Nos. 5,994,097 and 6,063,596.

GPCR mutations, both of the loss-of-function and of the activating variety, have been associated with numerous human diseases, Coughlin, supra. For example, retinitis pigmentosa may arise from either loss-of-function or activating mutations in the rhodopsin gene. Somatic activating mutations in the thyrotropin receptor cause hyperfunctioning thyroid adenomas, Parma, J. et al., Nature 365:649-651 (1993). Parma et al. indicate that it may be possible that certain G protein-coupled receptors susceptible to constitutive activation may behave as protooncogenes. Interestingly, GPCRs have functional homologues in human cytomegalovirus and herpesvirus, so GPCRs may have been acquired during evolution for viral pathogenesis, Strader et al., FASEB J., 9:745-754 (1995); Arvanitakis et al., Nature, 385:347-350 (1997); Murphy, Annu. Rev. Immunol. 12:593-633 (1994). The importance of the GPCR superfamily is further highlighted by the recent discoveries that some of its family members, the chemokine receptors CXCR4/Fusin and CCR5, are co-receptors for T cell-tropic and macrophage-tropic HIV virus strains, respectively, Alkhatib et al., Science, 272:1955 (1996); Choe et al., Cell, 85:1135 (1996); Deng et al., Nature, 381:661 (1996); Doranz et al., Cell, 85:1149 (1996); Dragic et al., Nature, 381:667 (1996); Feng et al., Science, 272:872 (1996). It is conceivable that blocking these receptors may prevent infection by the human immunodeficiency (HIV) virus. Other GPCR-related items include regulating cellular metabolism and diagnosing, treating and preventing particular diseases associated with particular GPCRs.

One important way to evaluate GPCRs and antibodies for GPCRs as novel drug targets and for other purposes such as diagnostics is through the creation and use of databases. Such databases can provide large amounts of information about genes, proteins, and other biological matter. An excellent example of such a database is the GPCR database created and maintained by LifeSpan BioSciences, Inc., Seattle, Wash., USA, which database is available by subscription to researchers and others needing such information. The information in the databases can, for example, be searched, compared, and analyzed. The compilation of such databases, as well as the searching, comparing, etc., of the databases, can be referred to as the field of “bioinformatics.” Investigations largely related to genes, such as the information found from the sequencing of the human genome, can be called “genomics” while similar activities on proteins can be called “proteomics.”

There has gone unmet a need for improved systems, compositions, methods, and the like relating to improved antigenicity of peptides from GPCRs and antibodies relating thereto. The present invention provides these and other advantages.

### SUMMARY

The present invention provides antigenic peptides for GPCRs and antibodies relating thereto, and related systems, methods, compositions, and the like, such as diagnostics and medicaments. Where antibodies against a given GPCR are not known, the present invention provides such antibodies, and preferred antigenic sequences for producing such antibodies. Where antibodies against a given GPCR are known, the present invention provides preferred antigenic peptides for producing antibodies that exhibit improved specificity, affinity or capacity to perform antibody-related actions relative to the known antibodies. The present invention also provides improved methods of selecting antigenic peptides from any desired protein or polypeptide, as well as antigenic peptides so produced and antibodies against such antigenic peptides.

The antigenic peptides and antibodies herein can be used, for example, to detect the presence or absence of corresponding GPCRs. They can be used to diagnose a variety of diseases and disorders in which GPCRs are involved, such as, e.g., immune-related diseases, cell growth-related diseases, cell regeneration-related diseases, immunological-related cell proliferative diseases, and autoimmune diseases. Examples of specific diseases include AIDS, allergies, Alzheimer's disease, amyotrophic lateral sclerosis, atherosclerosis, bacterial, fungal, protozoan and viral infections, benign prostatic hypertrophy, bone diseases (e.g., osteoarthritis, osteoporosis), carcinoma (e.g., basal cell carcinoma, breast carcinoma, embryonal carcinoma, ovarian carcinoma, renal cell carcinoma, lung adenocarcinoma, lung small cell carcinoma, pancreatic carcinoma, prostate carcinoma, transitional carcinoma of the bladder, squamous cell carcinoma, thyroid carcinoma), cardiomyopathy, chronic and acute inflammation, circadian rhythm disorders, COPD, Crohn's disease, diabetes, Duchenne muscular dystrophy, embryonal carcinoma, endotoxic shock, environmental stress (e.g., by heat, UV or chemicals), gastrointestinal disorders, glioblastoma multiform, graft vs. host disease, Hodgkin's disease, inflammatory bowel disease, ischemia, stroke, lymphoma, macular degeneration, malignant cytokine production, malignant fibrous histiocytoma, melanoma, meningioma, mesothelioma, multiple sclerosis, nasal congestion, pain, Parkinson's disease, prostate carcinoma, psoriasis, rhabdomyosarcoma, psychotic or neurological disorders (e.g., anxiety, depression, schizophrenia, dementia, mental retardation, memory loss, epilepsy, locomotor problems, respiratory disorders, asthma, eating/body weight disorders including obesity, bulimia, diabetes, anorexia, nausea, hypertension, hypotension), renal disorders, reperfusion injury, rheumatoid arthritis, sarcoma (e.g., chondrosarcoma, Ewing's sarcoma, osteosarcoma), septicemia, seminoma, sexual/reproductive disorders, tonsil, transitional carcinoma of the bladder, transplant rejection, trauma, tuberculosis, ulcers, ulcerative colitis, urinary retention, vascular and cardiovascular disorders, or any other disease or disorder in which G protein-coupled receptors are involved, as well as learning and/or memory disorders, diabetes, pain perception disorders, anorexia, obesity, hormonal release problems, or any other disease or disorder in which a specific GPCR is involved.

The association of particular GPCRs with particular diseases, disorders or conditions will be apparent to a person of ordinary skill in the art in view of the present application, and thus the association with the antibodies of the present invention to the corresponding diseases, disorders or conditions.

Thus, in one aspect the present invention provides isolated antigenic peptides according to any one of SEQ ID NOS. 692-2292. The isolated antigenic peptides also comprise an amino acid sequences that are at least about 90% or 95% identical to such sequences, or be an analog of such sequences, or comprise a short antigenic amino acid sequence that is identical to at least 5 consecutive amino acids set forth in any one of such sequences or contain no more than one conservative amino acid substitution over at least 7 consecutive amino acids set forth in any of such sequences. The present invention also provides antibodies, particularly isolated antibody having high specificity and high affinity or avidity for a particular GPCR or other target polypeptide or protein, generated using the antigenic peptides discussed herein.

The present invention also provides isolated nucleic acid molecules encoding an antigenic peptide or antibody as described herein. The molecule can encode a naturally occurring human antigenic peptide. In some embodiments, the present invention provides processes for producing an isolated polynucleotide can comprise hybridizing a nucleotide encoding an antigenic peptide as discussed herein to DNA such as genomic DNA under stringent or highly stringent conditions and isolating the polynucleotide detected with the nucleotide.

The present invention also provides kits and assays, such as kits for the detection of antibodies against a particular GPCR or other target polypeptide in a sample comprising: a) an isolated antigenic peptide as discussed herein and derived from the particular GPCR, and b) at least one of a reagent or a device for detecting the antibodies, or comprising: a) an isolated antibody as described herein, and b) at least one of a reagent or a device for detecting the antibody. The assays include detection of a particular GPCR in a sample, comprising: a) providing an isolated antigenic peptide, b) contacting the isolated antigenic peptide corresponding to the particular GPCR with the sample under conditions suitable and for a time sufficient for the antigenic peptide to bind to one or more antibodies specific for the target protein present in the sample, to provide an antibody-bound target protein, and c) detecting the antibody-bound antigenic peptide, and therefrom determining whether the sample contains the particular GPCR. The assays can further comprise the step of binding the isolated antigenic peptide or the antibody to a solid substrate, and the sample can be an unpurified sample, for example from a human being.

The assay can be selected from the group consisting of a countercurrent immuno-electrophoresis (CIEP) assay, a radioimmunoassay, a radioimmunoprecipitation, an enzyme-linked immuno-sorbent assay (ELISA), a dot blot assay, an inhibition or competition assay, a sandwich assay, an immunostick (dip-stick) assays, a simultaneous assay, an immunochromatographic assay, an immunofiltration assay, a latex bead agglutination assay, an immunofluorescent assay, a biosensor assay, and a low-light detection assay.

In other aspects, the present invention provides methods of identifying an amino acid sequence for an antigenic peptide from a candidate polypeptide sequence such as a polypeptide or protein wherein the antigenic peptide has a length of about 5 to about 100 amino acids, typically 6 amino acids to about 50 amino acids, and preferably 7 amino acids to about 20 amino acids. The methods comprise: a) searching the candidate polypeptide sequence using a comparison window of the length, and b) selecting against amino acid sequences of the length and having at least 1 to 3 or 4 characteristics selected from the group consisting of 1) at least two consecutive prolines, 2) at least two consecutive serines, 3) at least two consecutive lysines, 4) at least two consecutive arginines, 5) at least two consecutive aspartic acids, 6) at least two consecutive glutamic acids, 7) methionine, 8) tryptophan, and 9) at least five consecutive amino acids comprising no charged amino acids. Preferably, the method comprises selecting against at least 5 to all of the characteristics.

The methods can comprise, independently or in addition, selecting against amino acid sequences of the desired length having at least one of the following characteristics 1) sequences having at least 5 consecutive amino acids that are identical to an alternative amino acid sequence from an alternative polypeptide that can be different from the candidate polypeptide, 2) posttranslational modification sites, and 3) highly hydrophobic sequences. The posttranslational modification sites can be phosphorylation or glycosylation sites. The methods can also comprise performing a BLAST-type or a FAST-type analyses for the candidate polypeptide sequence.

These and other aspects, features, and embodiments are set forth within this application, including the following Detailed Description and attached drawings. The present invention comprises a variety of aspects, features, and embodiments; such multiple aspects, features, and embodiments can be combined and permuted in any desired manner. In addition, various references are set forth herein, including in the Cross-Reference To Related Applications, that discuss certain compositions, apparatus, methods, or other information; all such references are incorporated herein by reference in their entirety and for all their teachings and disclosures, regardless of where the references may appear in this application.

### BRIEF DESCRIPTION OF THE DRAWING

FIG. 1 depicts representative examples of the nucleotide and amino acid sequences of the GPCRs for which antigenic peptides are set forth herein, SEQ ID NOS. 1-691.

FIG. 2 depicts amino acid sequences for the antigenic peptides for the GPCRs herein, SEQ ID NOS. 692-2292.

FIG. 3 depicts a listing of GPCRS for which commercially available antibodies are putatively available.

### DETAILED DESCRIPTION

A. Introduction and Overview

Diseases such as immune-related diseases, cell growth-related diseases, cell regeneration-related diseases, immunological-related cell proliferative diseases, and autoimmune diseases are serious health problems in the modem world. Any improvement in the diagnosis, treatment or other remediation of such diseases is a significant advance for millions of people. The present invention provides methods of identifying and selecting desirable antigenic peptides for GPCRs and other desired target or candidate proteins and polypeptides. The present invention also provides the antigenic peptides themselves, as well as antibodies against the antigenic peptides (and against proteins or polypeptides containing such antigenic peptides), and related diagnostics, antibody-based therapeutics directed to certain diseases and conditions, and other helpful compositions, systems, kits, assays and the like. The compositions, methods, and the like can be useful, for example, as agonists, antagonists, probes, and otherwise as may be desired.

The antigenic peptides have been carefully selected using specific selection criteria and methodologies set forth herein to take advantage of particularly advantageous regions of the GPCRs from which they have been derived to provide unusually specific and immunogenic antigens. These antigenic peptides are particularly useful for producing highly specific antibodies against the antigenic peptides, which, in turn, also means antibodies that are highly specific for the corresponding GPCRs containing the antigenic peptides. Accordingly, the antigenic peptides of the present invention, and the antibodies produced therefrom, are particularly useful for high specifity, low noise diagnostics and, in the case of the antibodies, for certain antibody-based therapeutics, as well as methods, kits, systems, and the like incorporating or based on such antigenic peptides or antibodies.

The antibodies produced using the antigenic peptides of the present invention, for example, have a specificity for the corresponding GPCR such that the antibodies can selectively detect the corresponding GPCR in a sample containing non-desired or contaminating proteins or polypeptides, such as a tissue or blood sample. Preferably, the antibodies have a high specificity such that no significant amounts of such proteins or polypeptides are detected, and further preferably have a specificity such that only insubstantial to essentially zero amounts of non-desirable proteins are detected.

The antibodies produced using the antigenic peptides of the present invention, for example, typically have an affinity or avidity constant (Ka) of at least about 10⁷ liters/mole, typically a high affinity or avidity at least about 10⁹ liters/mole, preferably at least about 10¹⁰ liters/mole, and further preferably at least about 10¹¹ liters/mole.

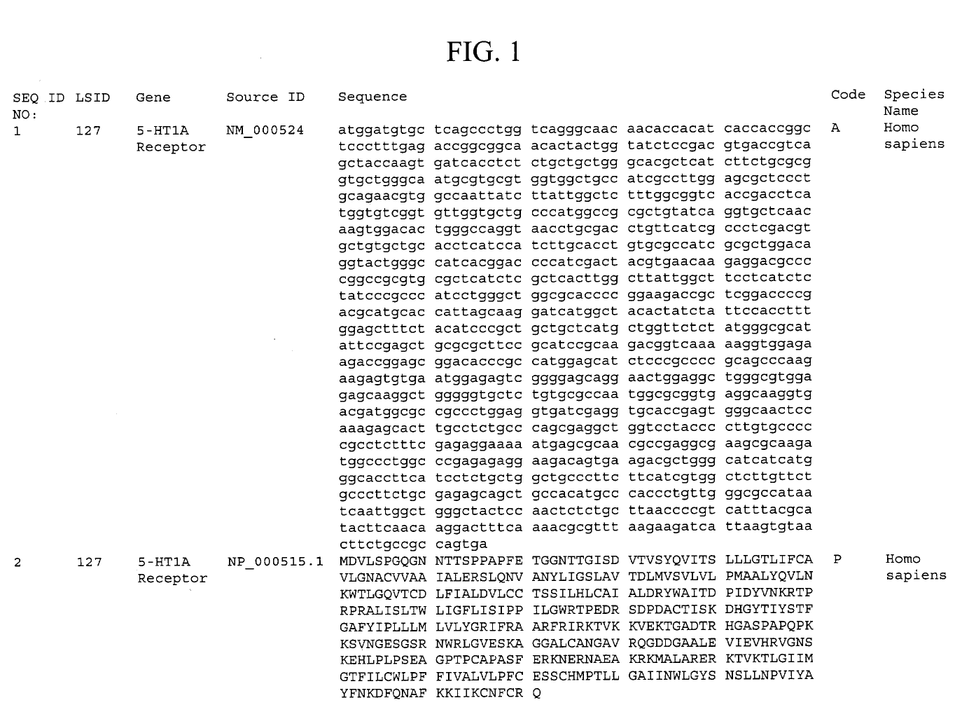

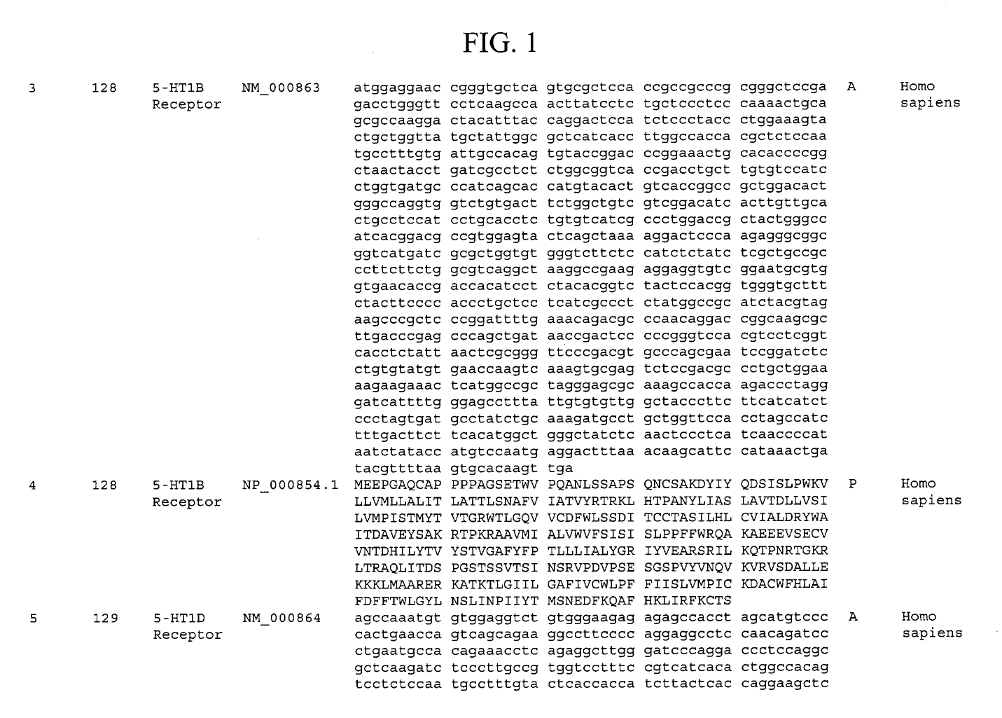

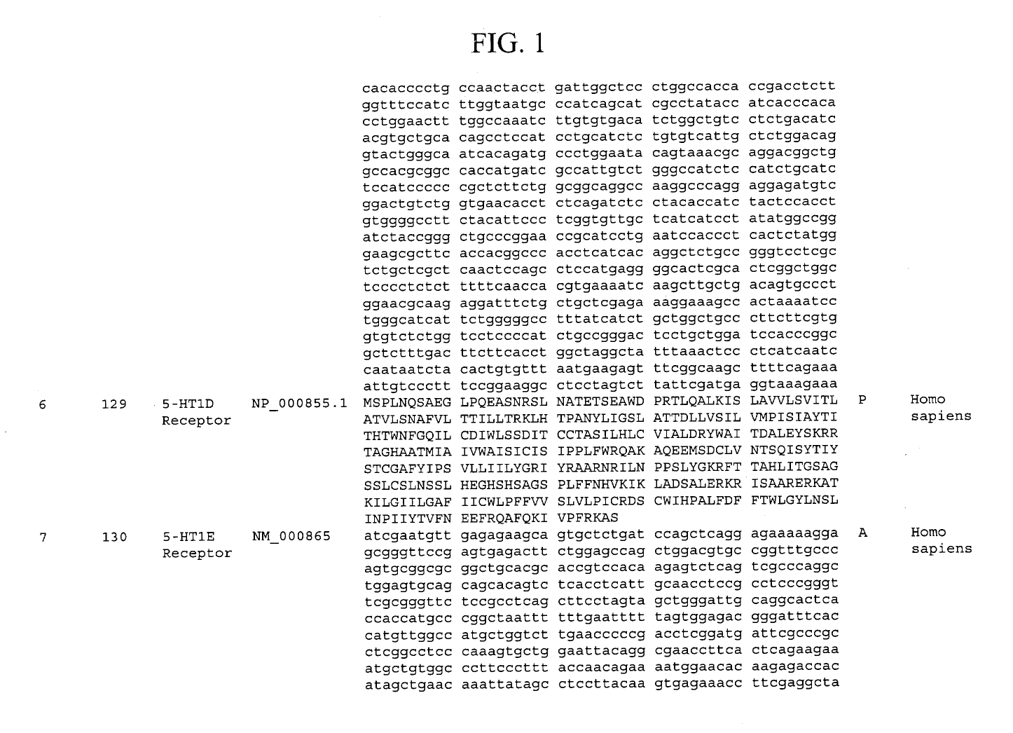

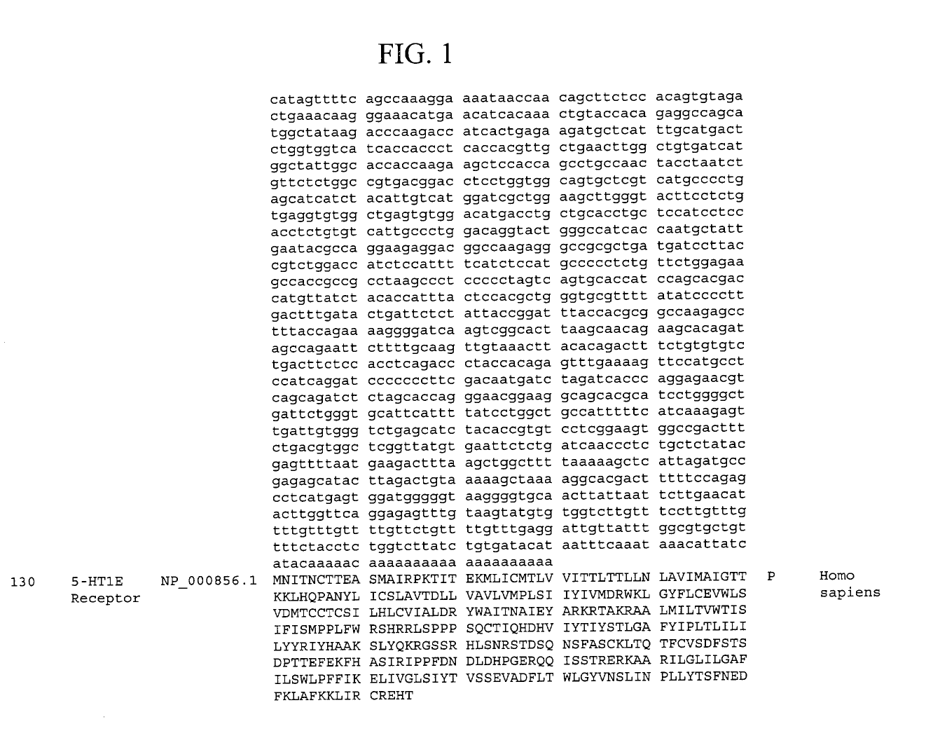

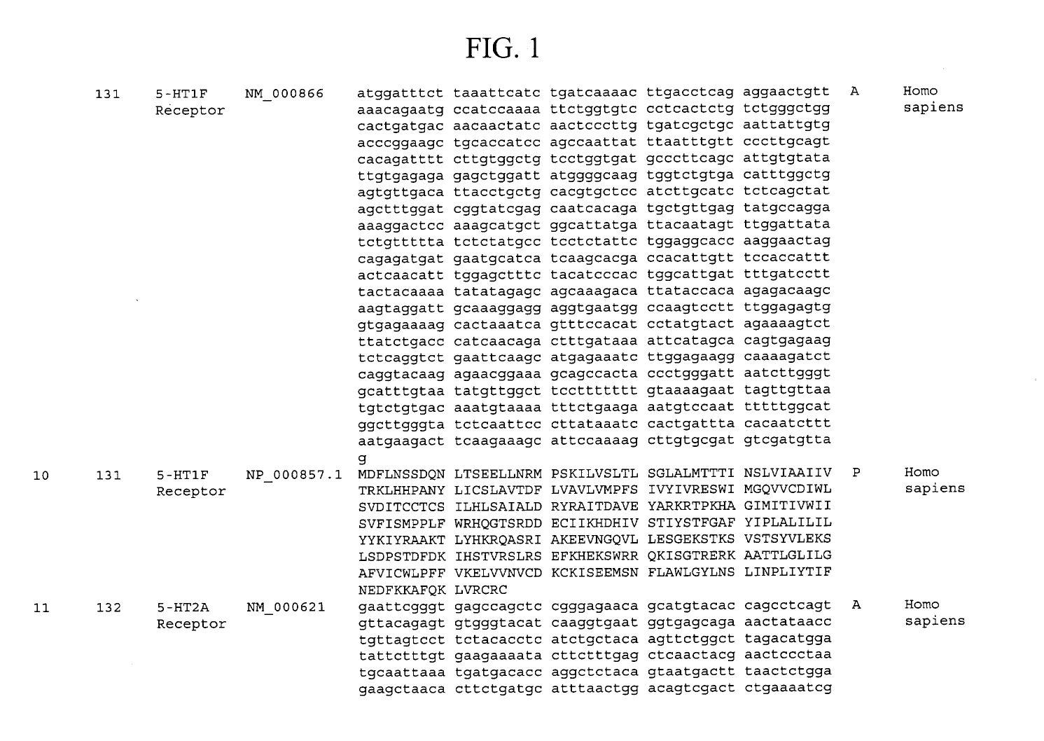

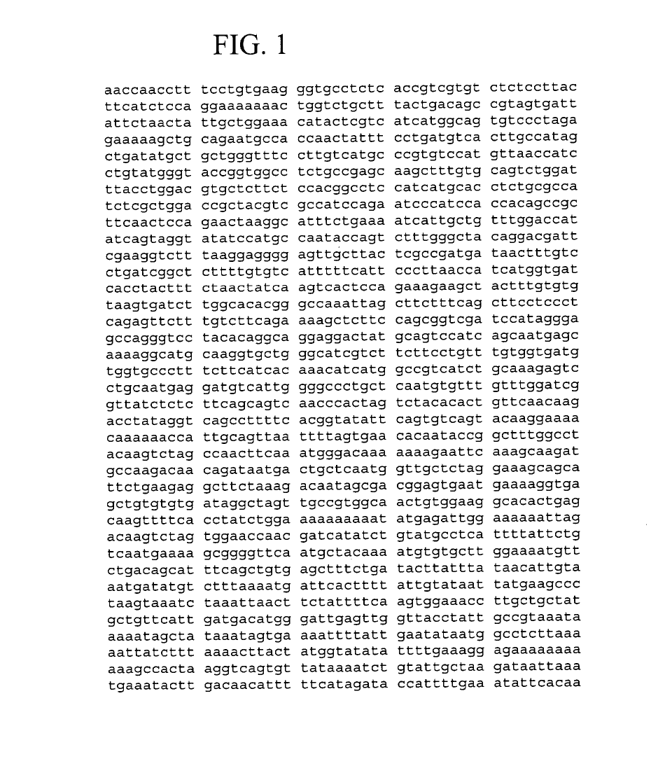

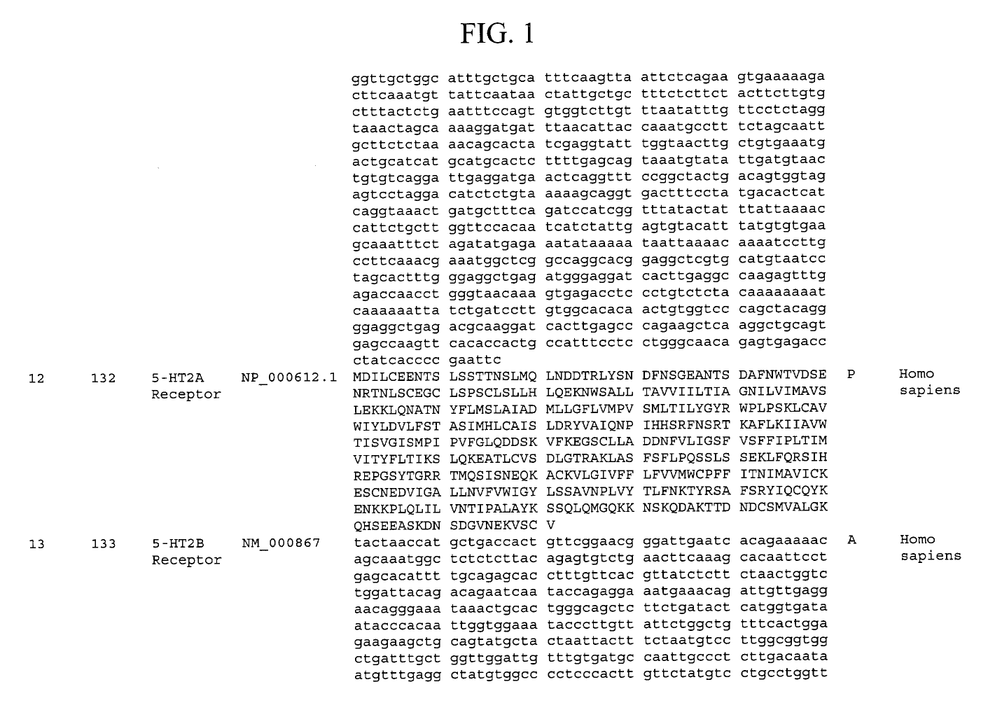

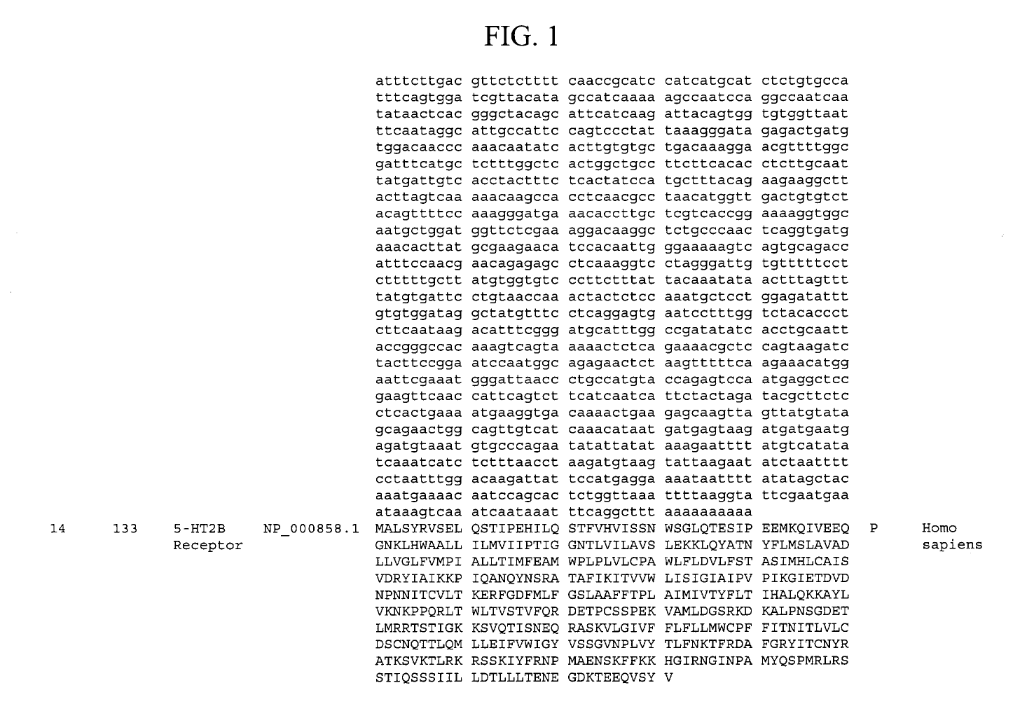

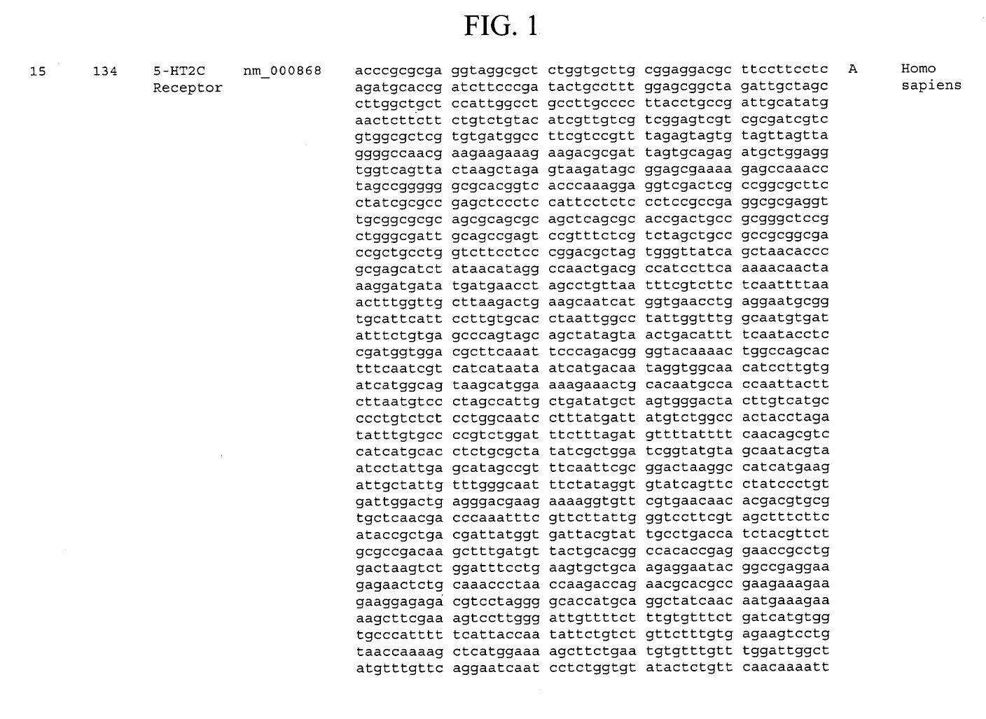

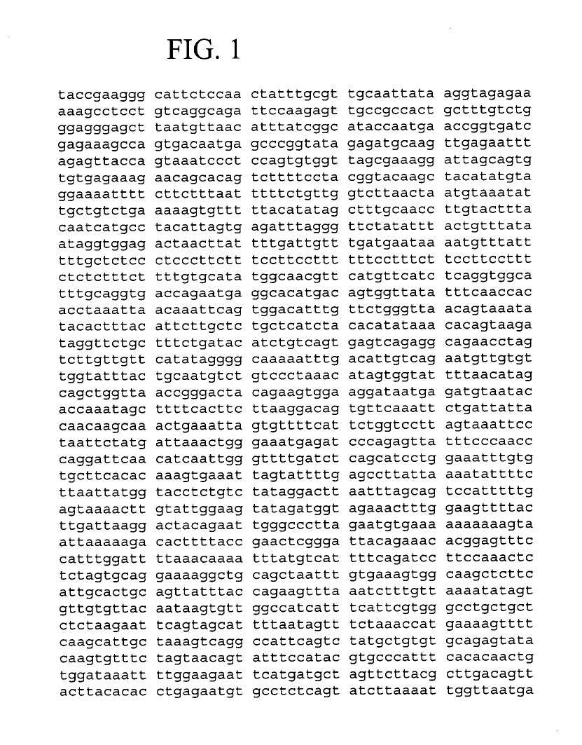

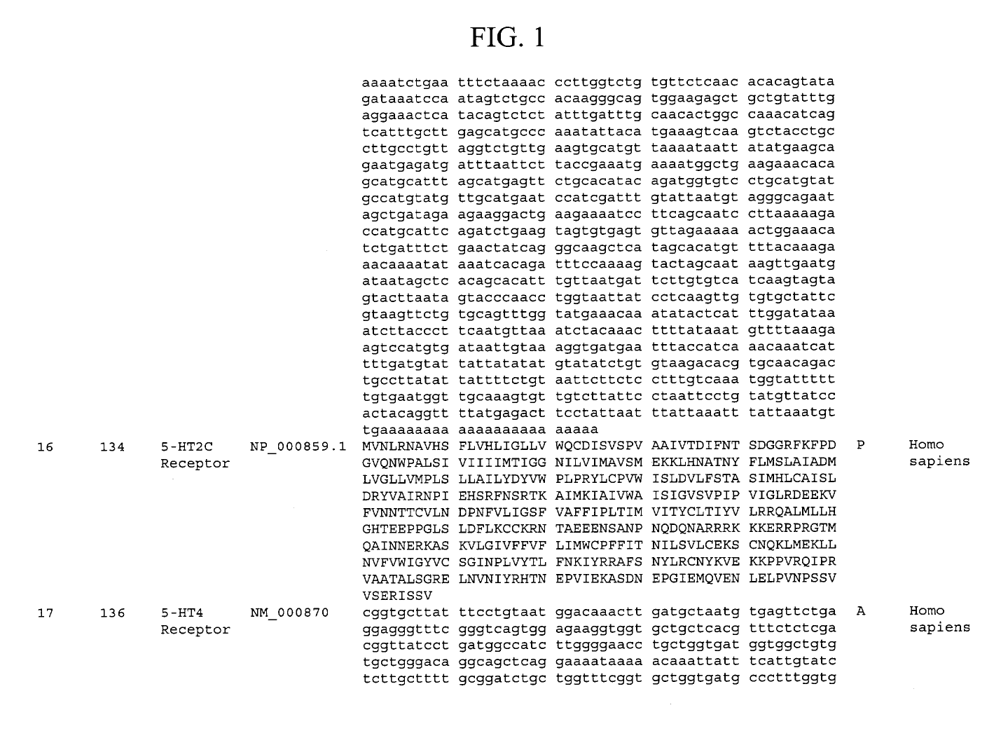

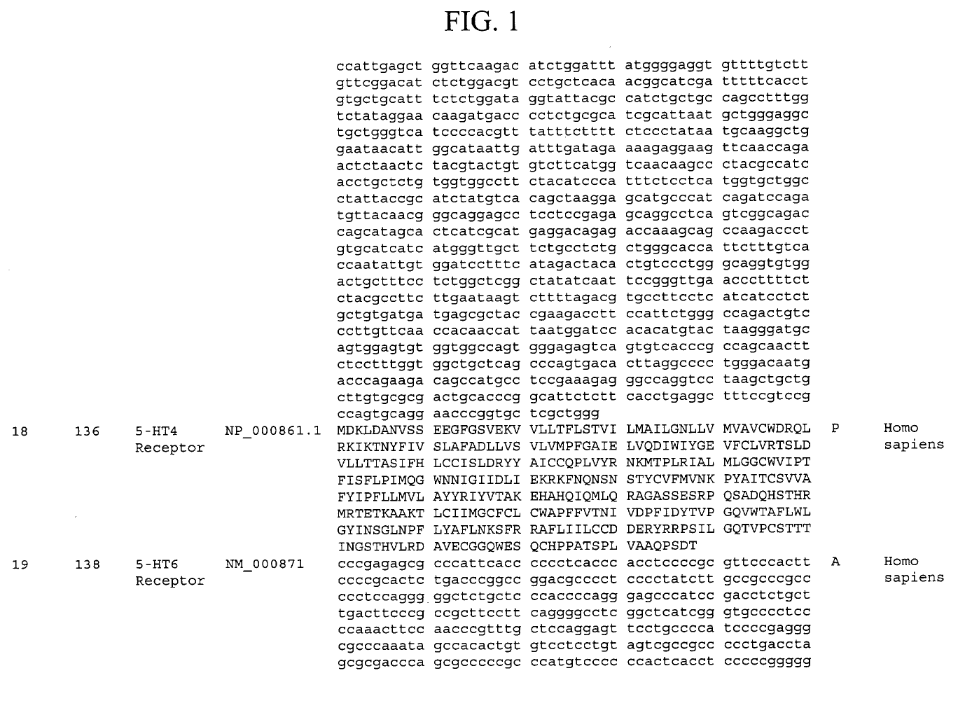

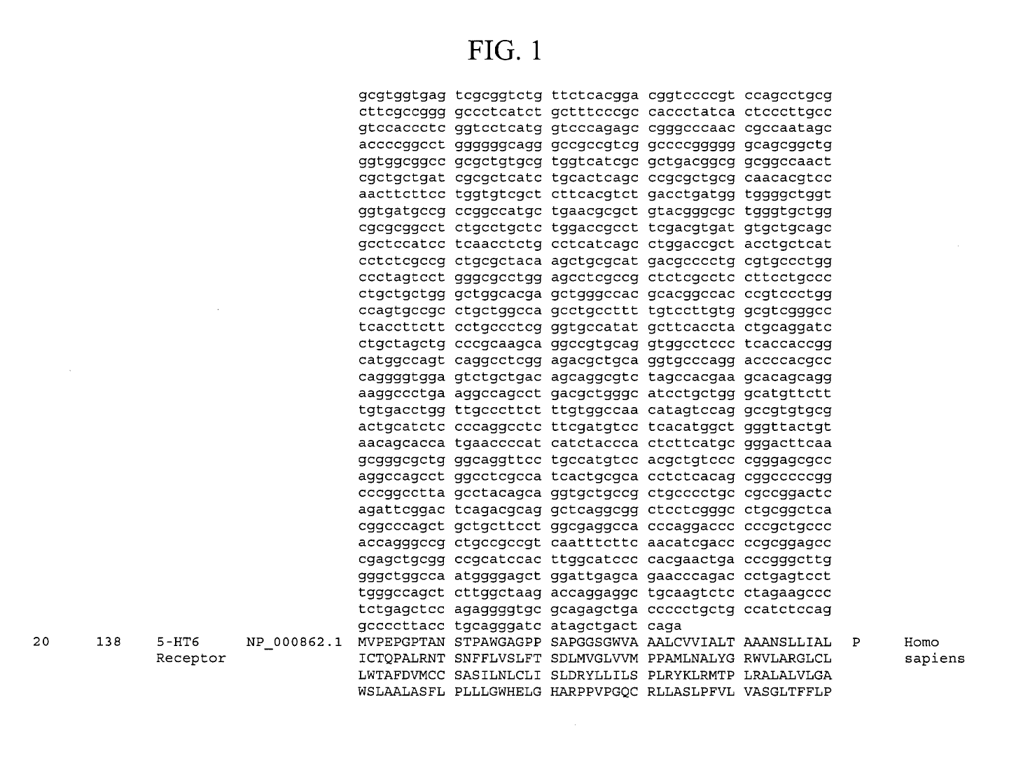

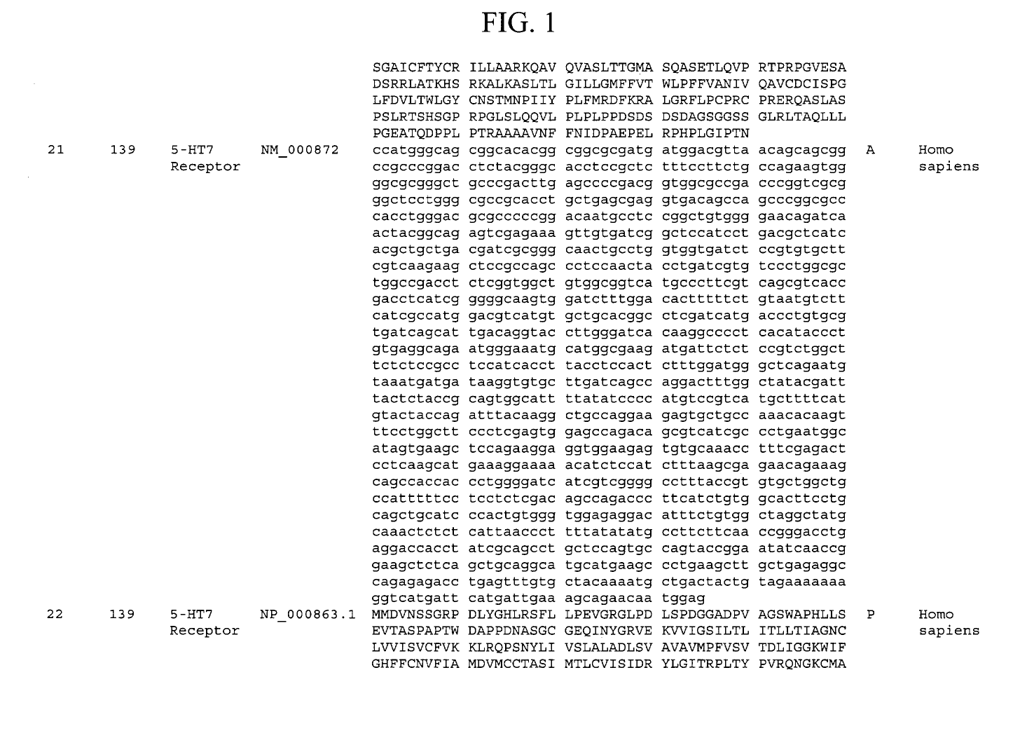

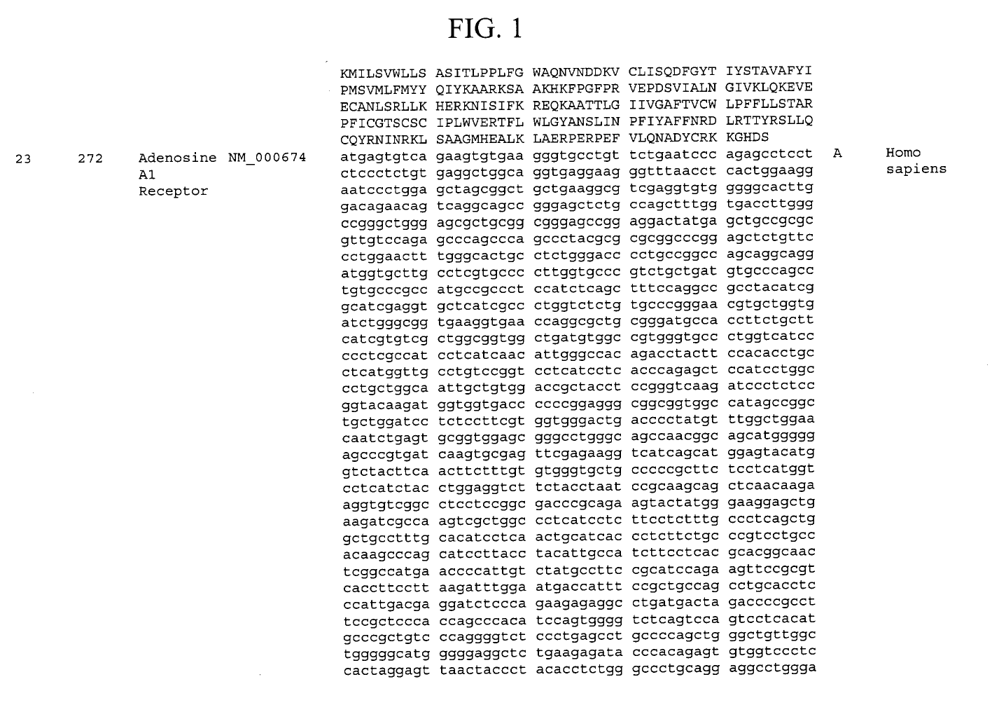

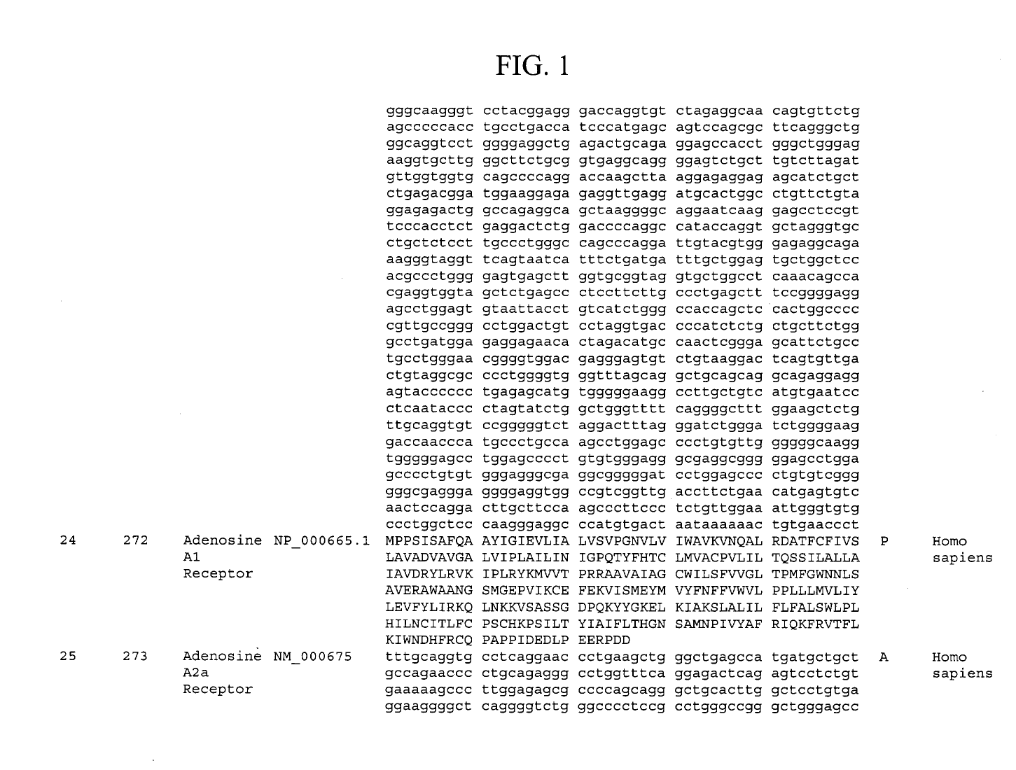

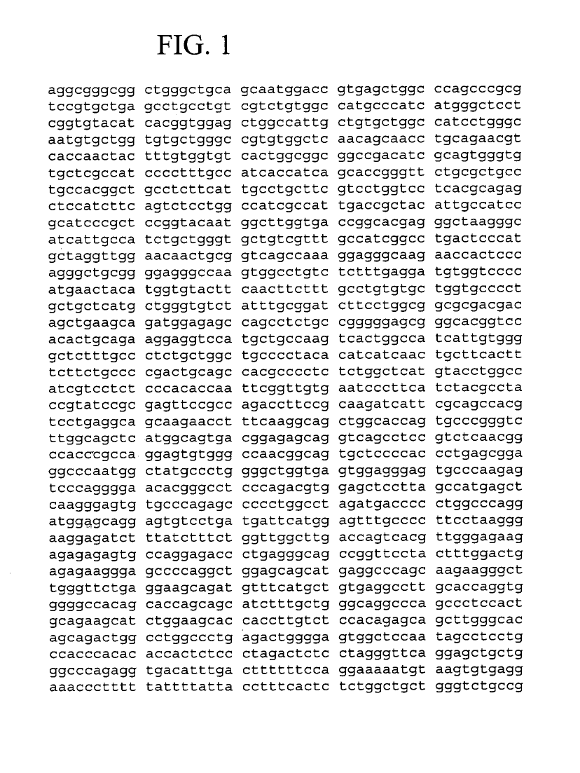

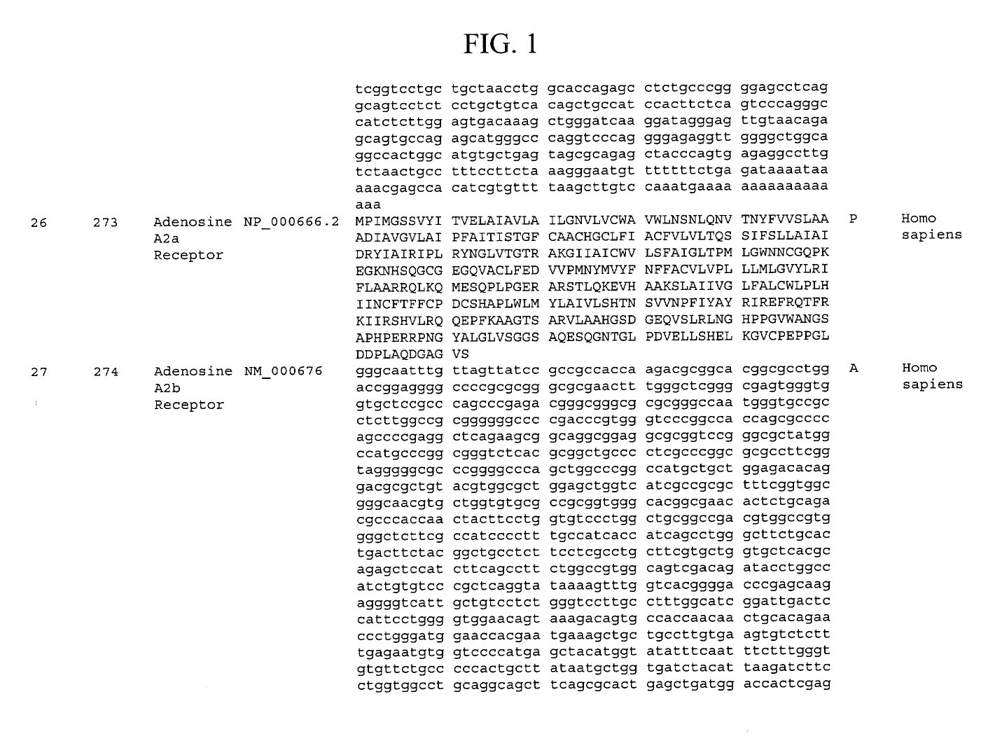

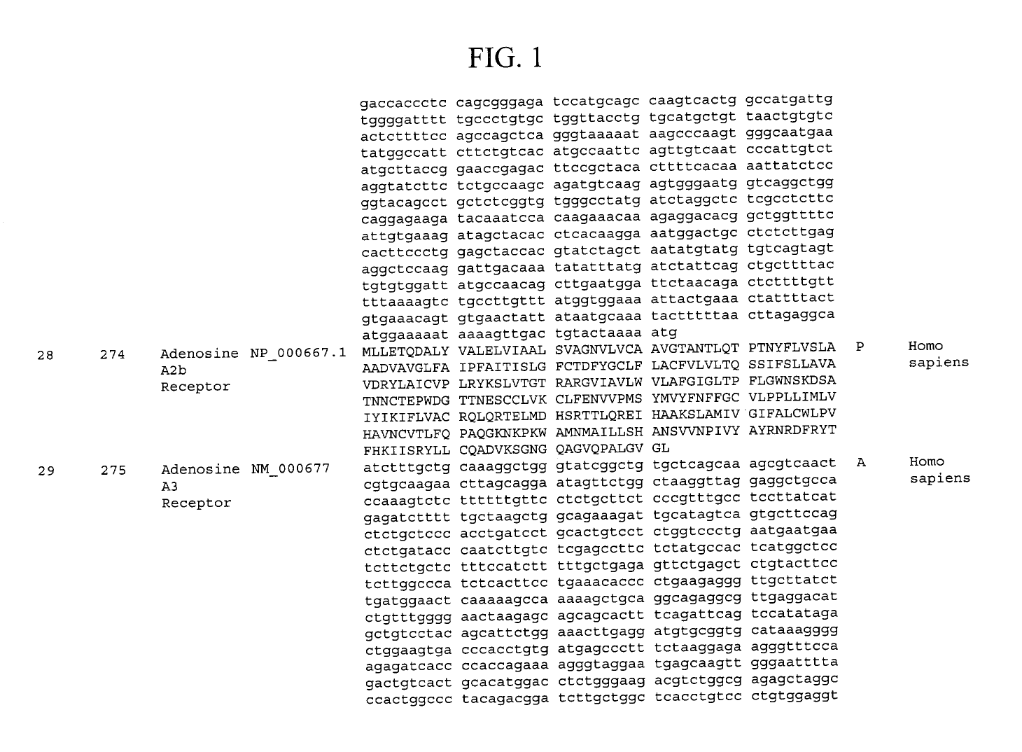

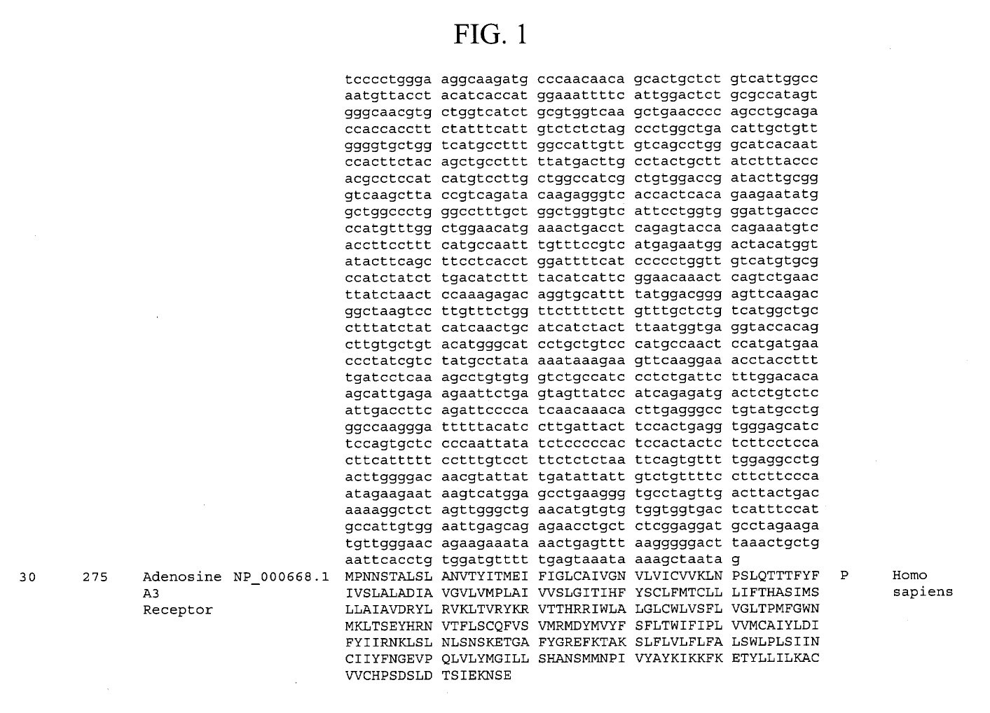

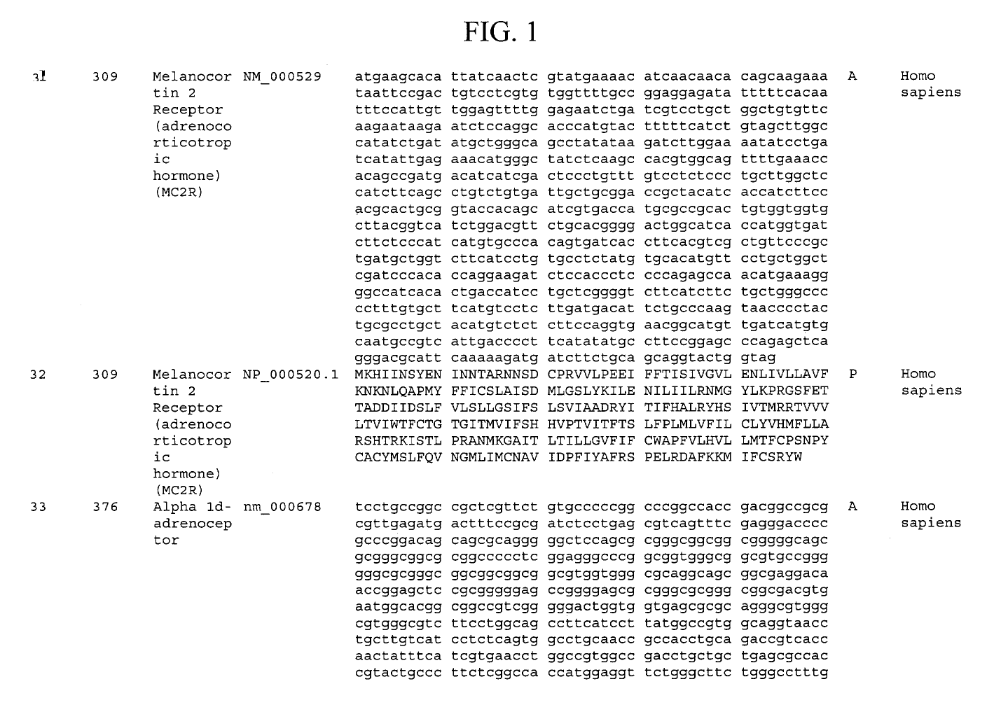

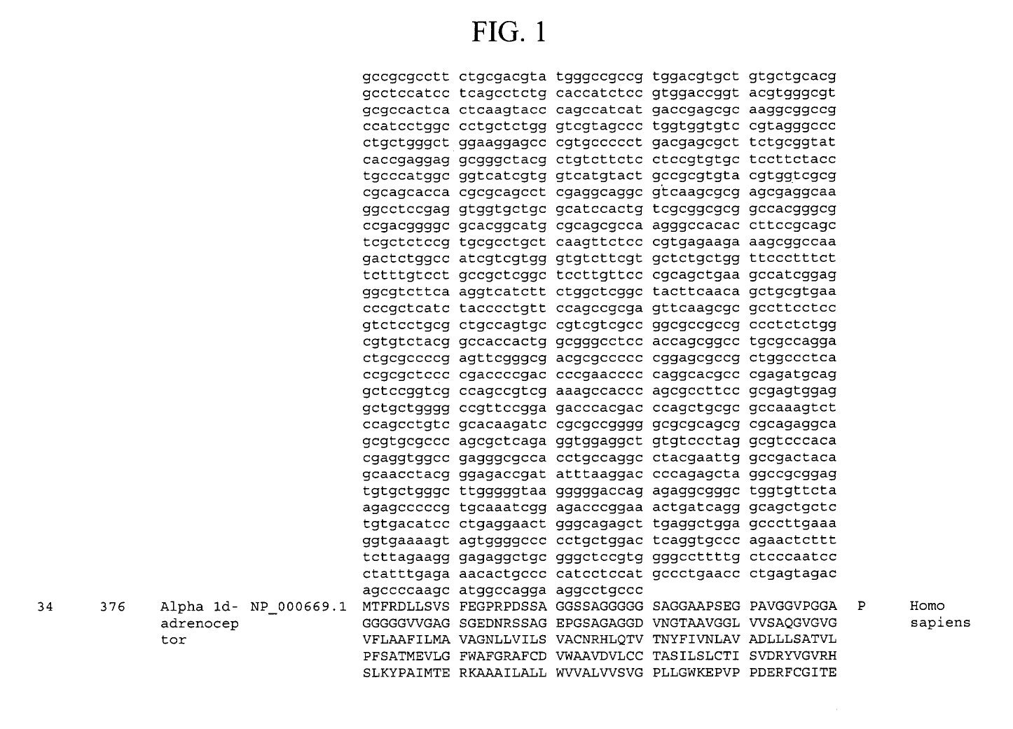

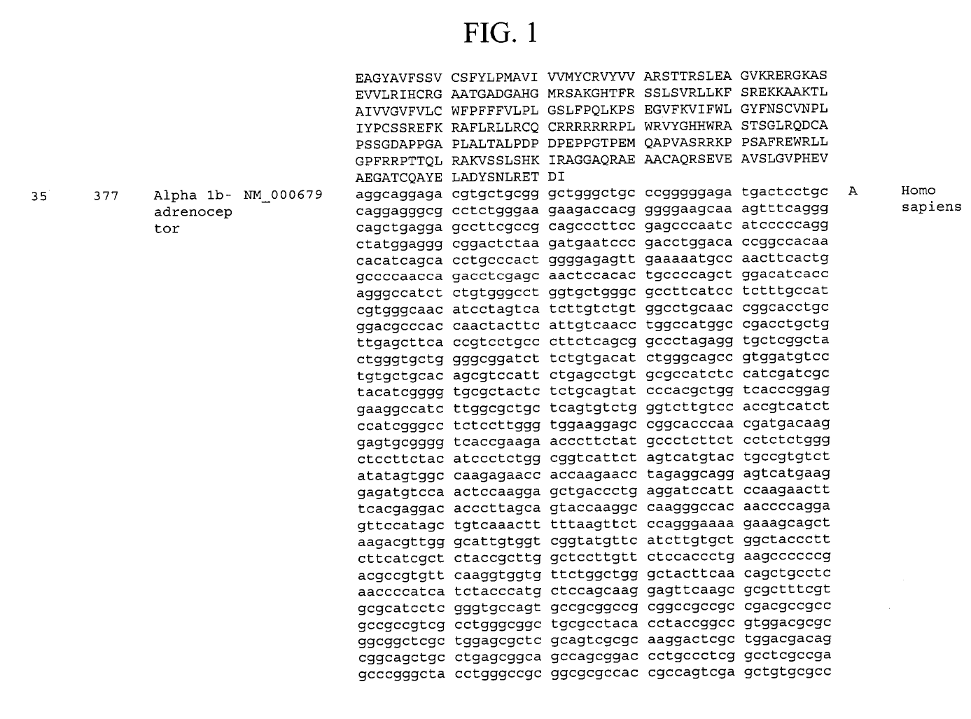

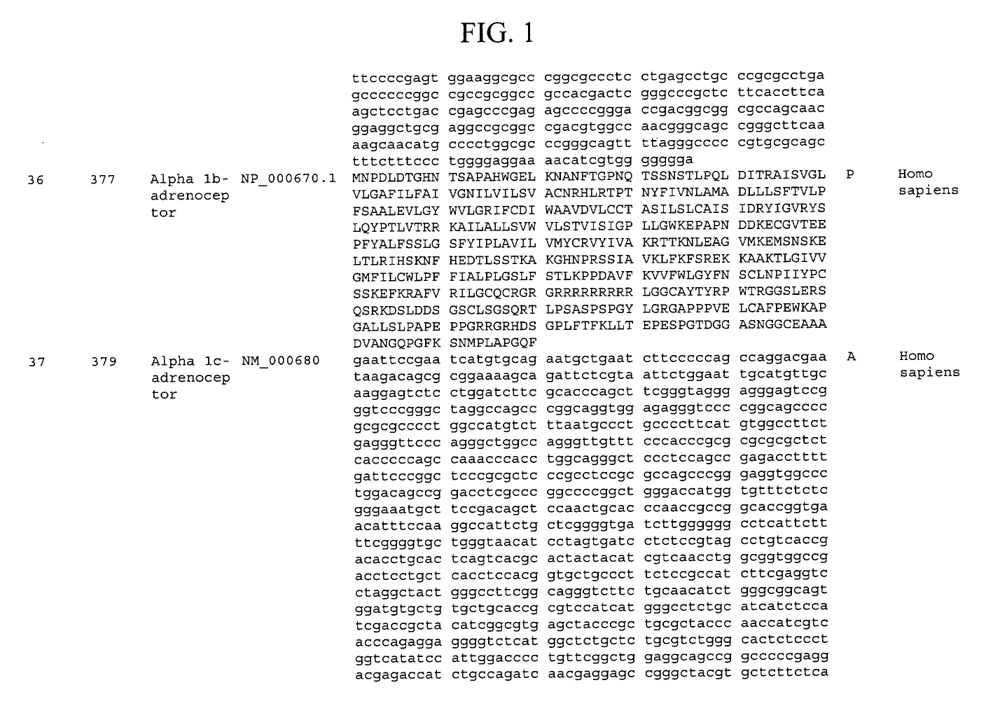

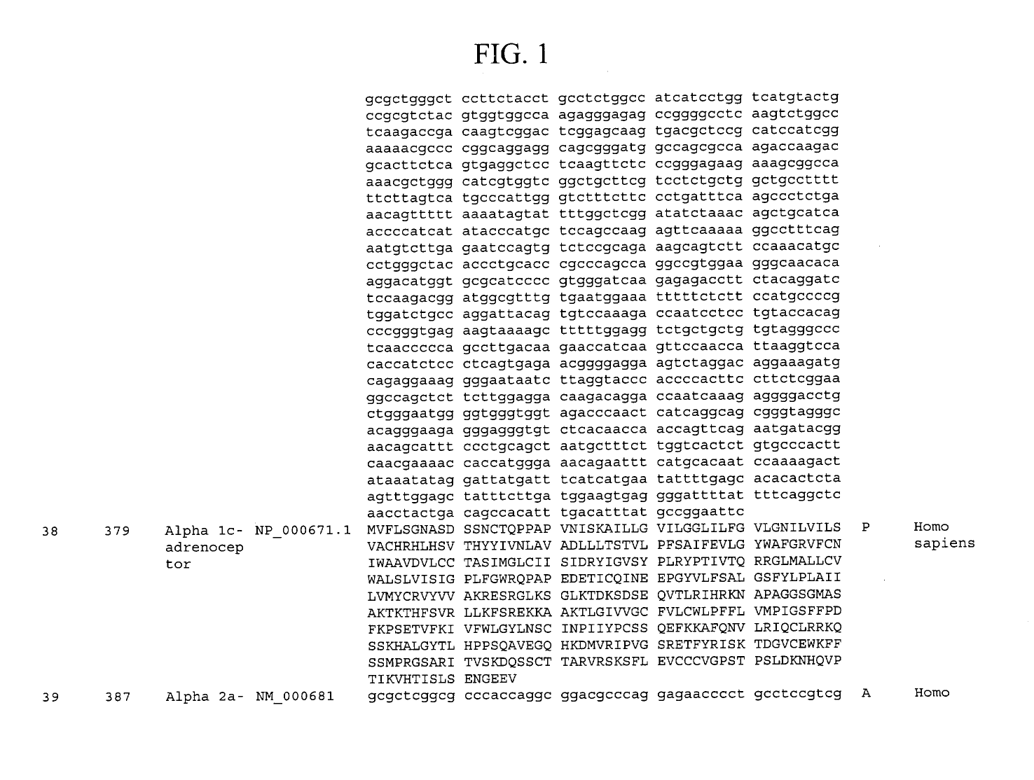

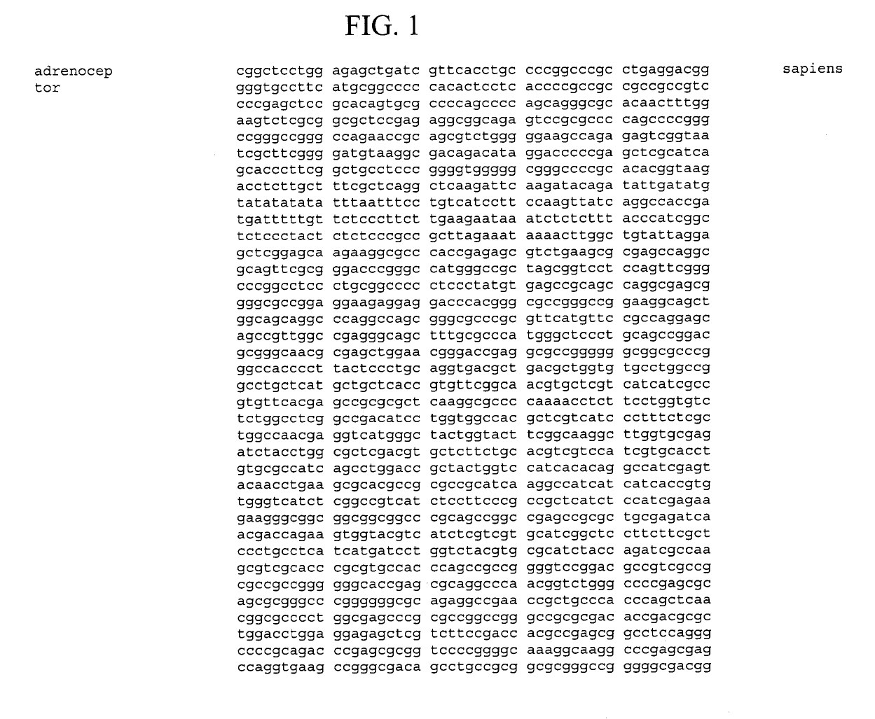

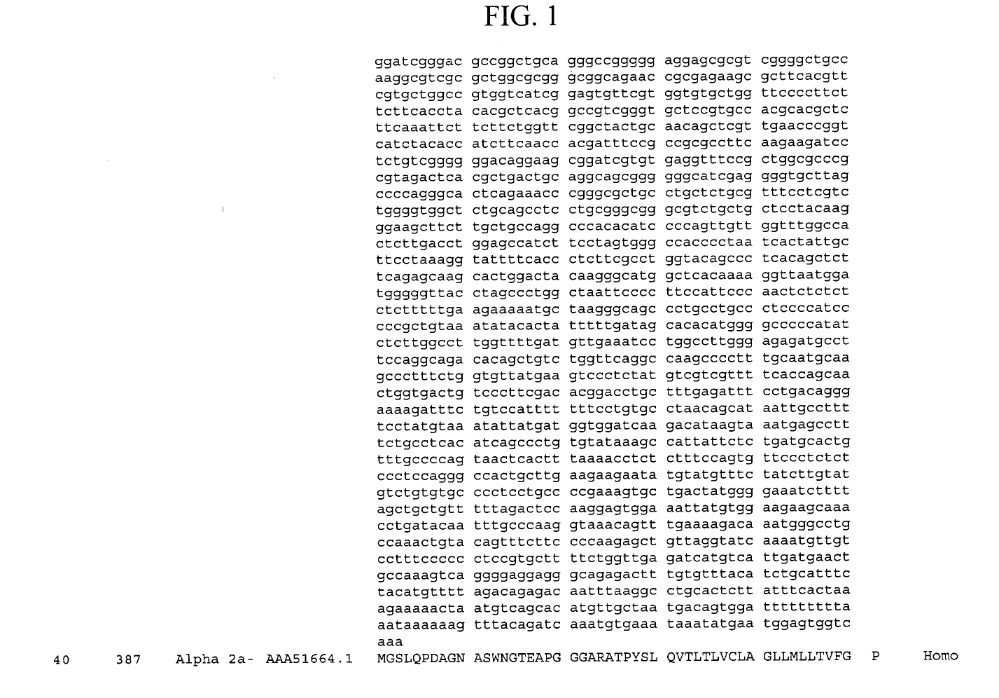

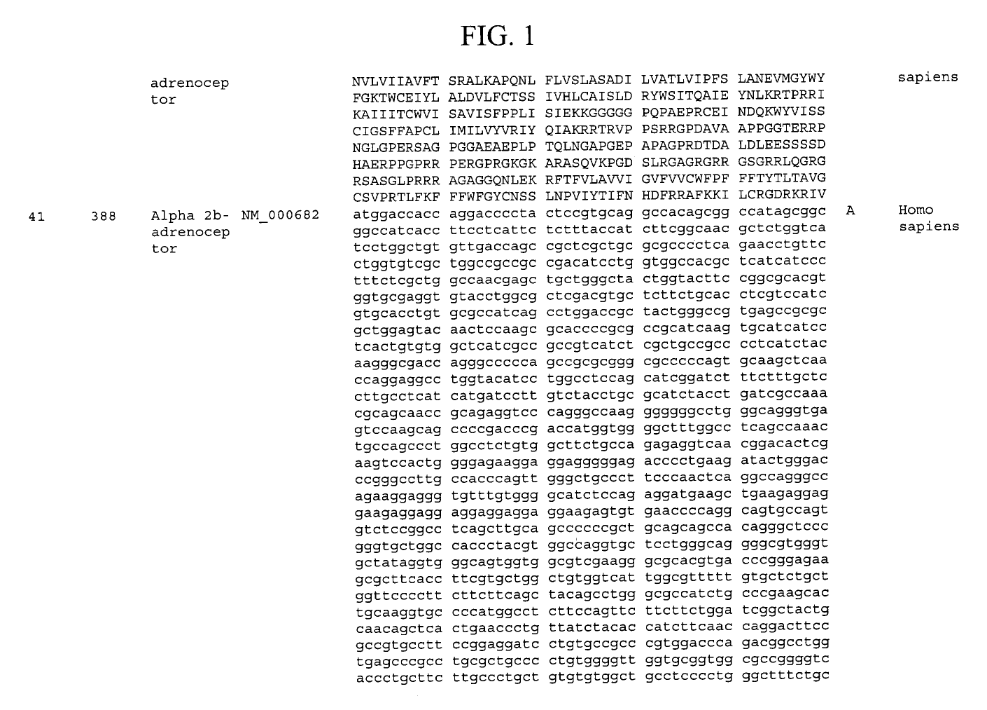

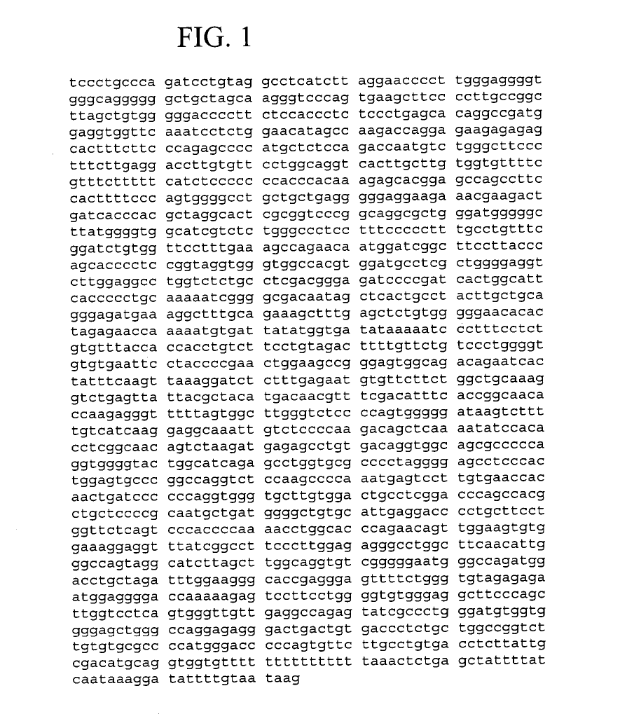

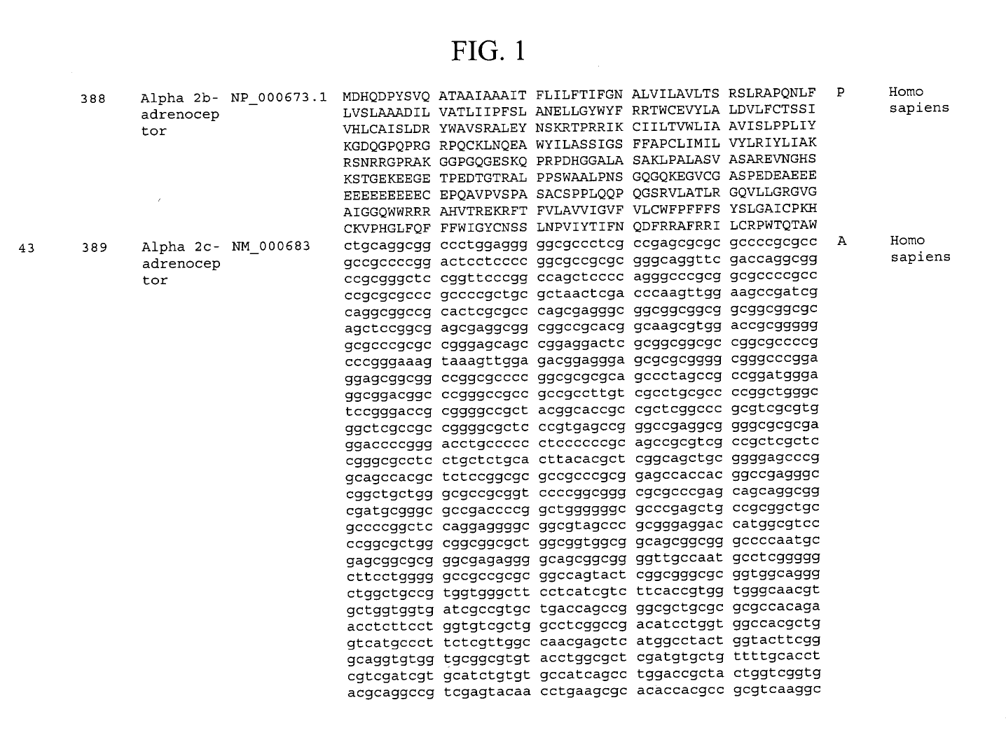

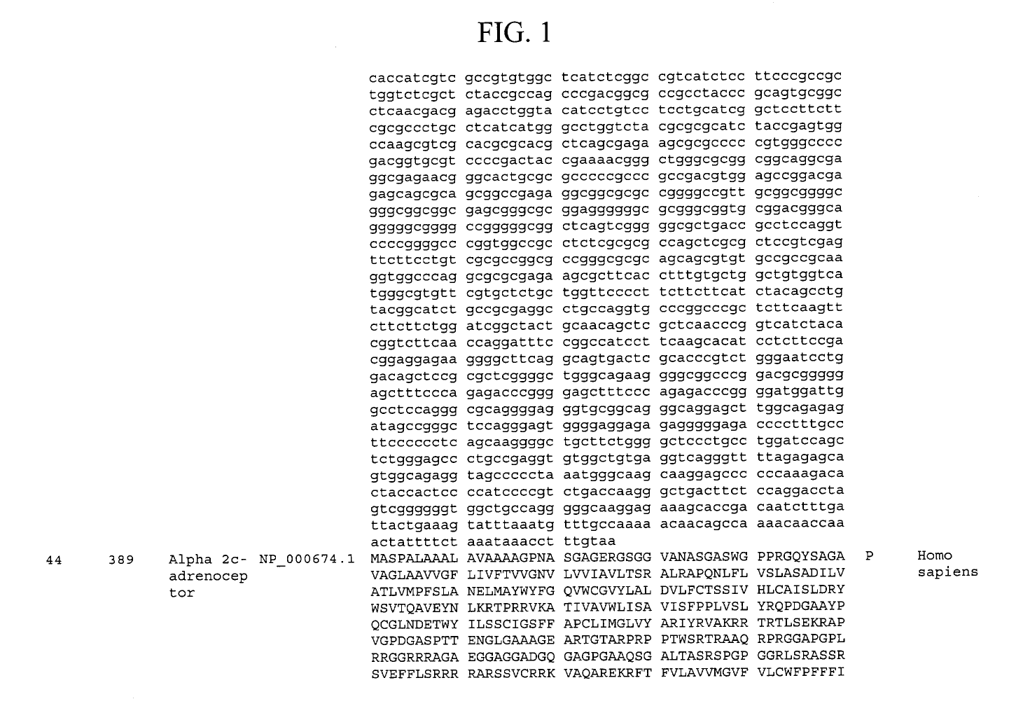

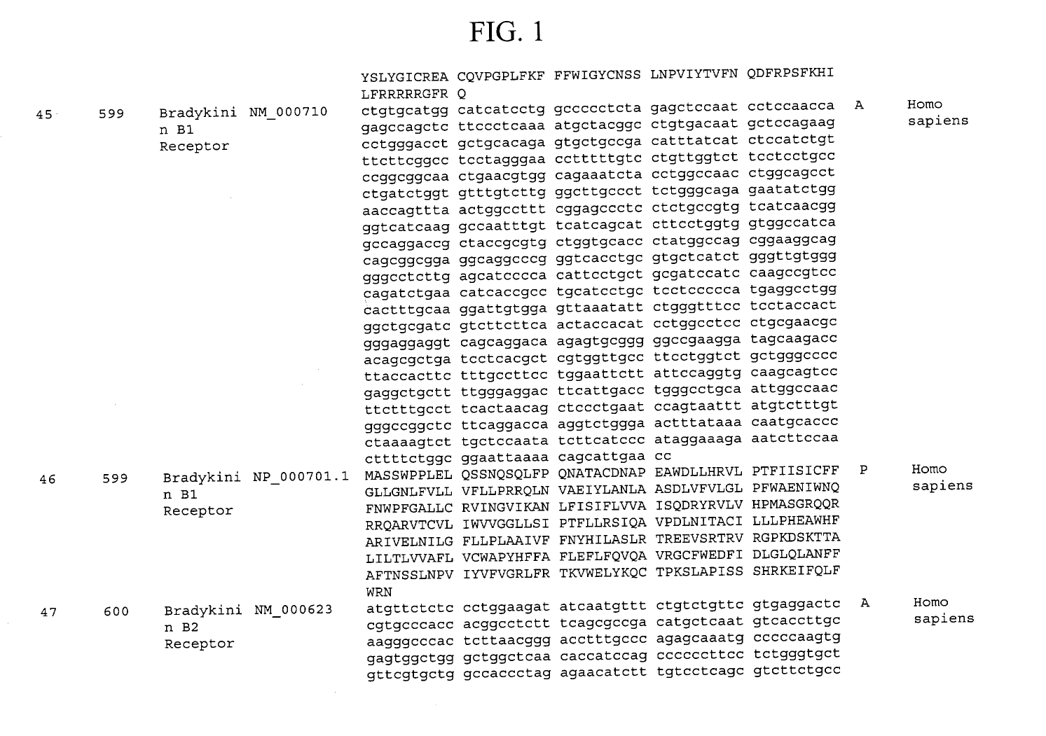

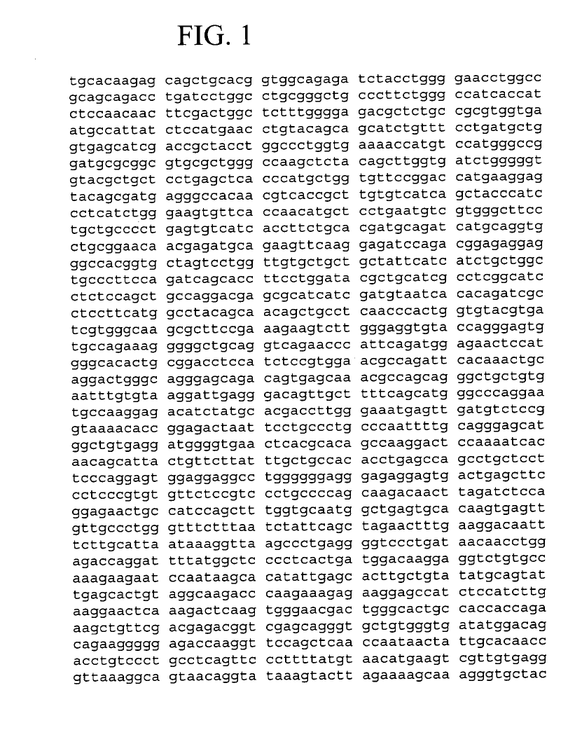

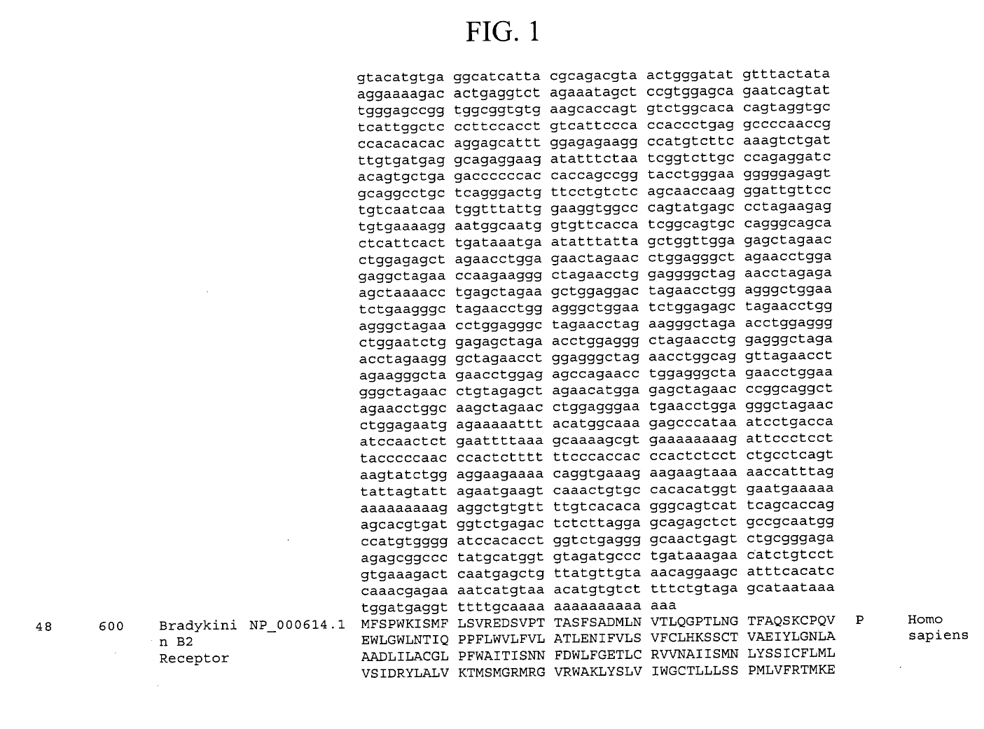

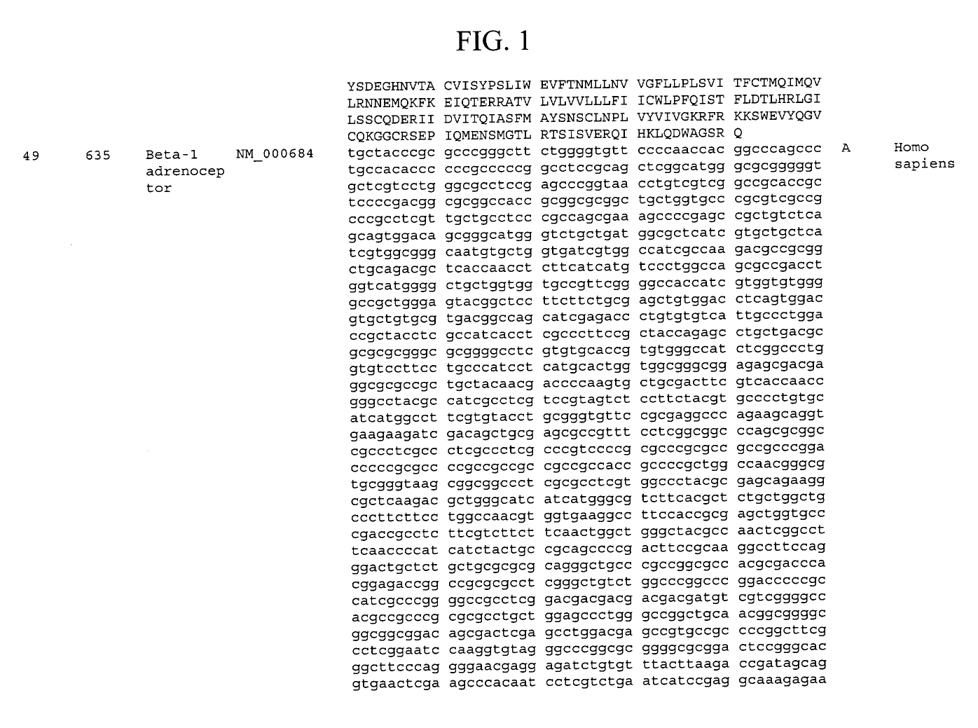

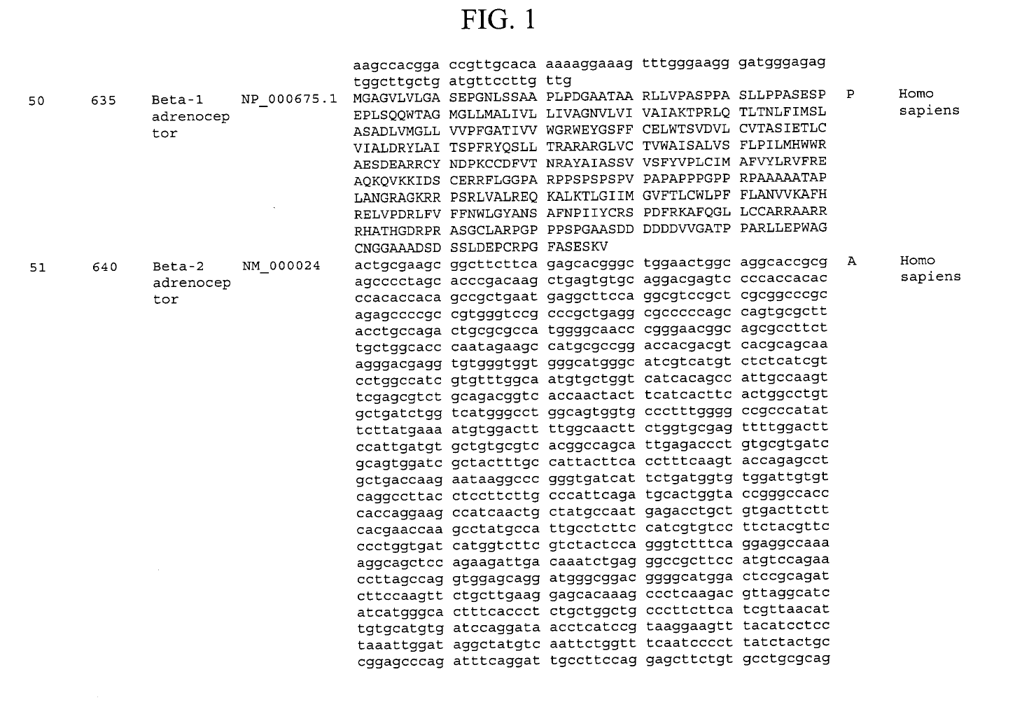

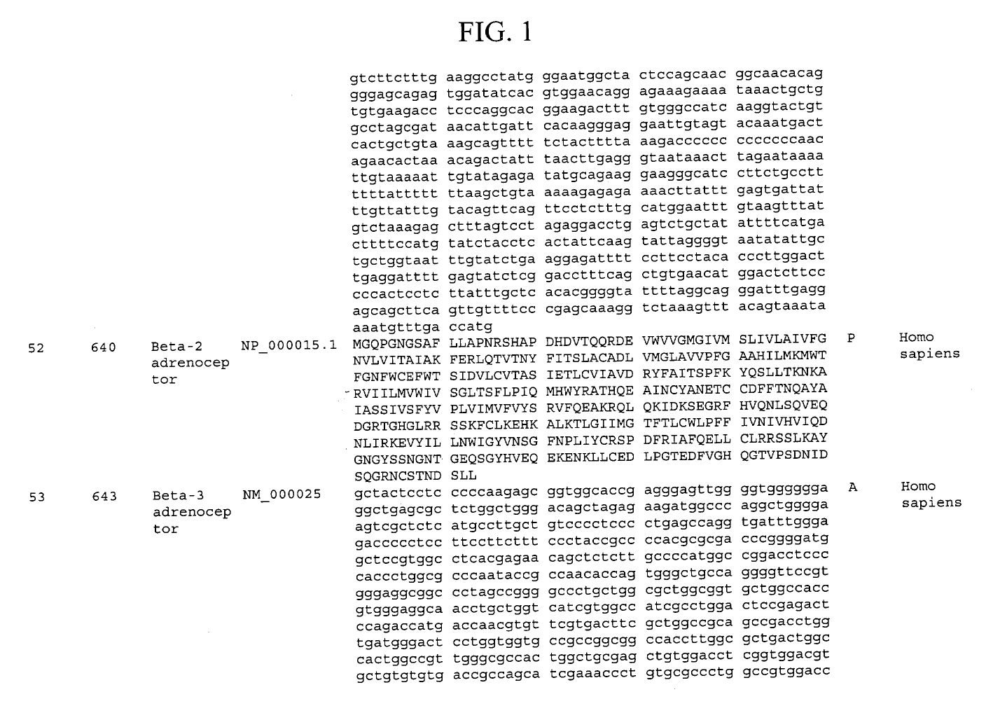

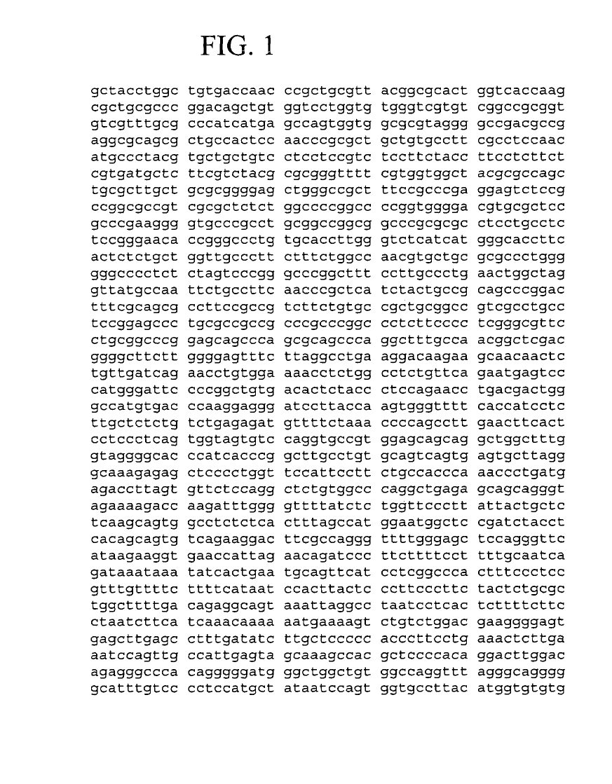

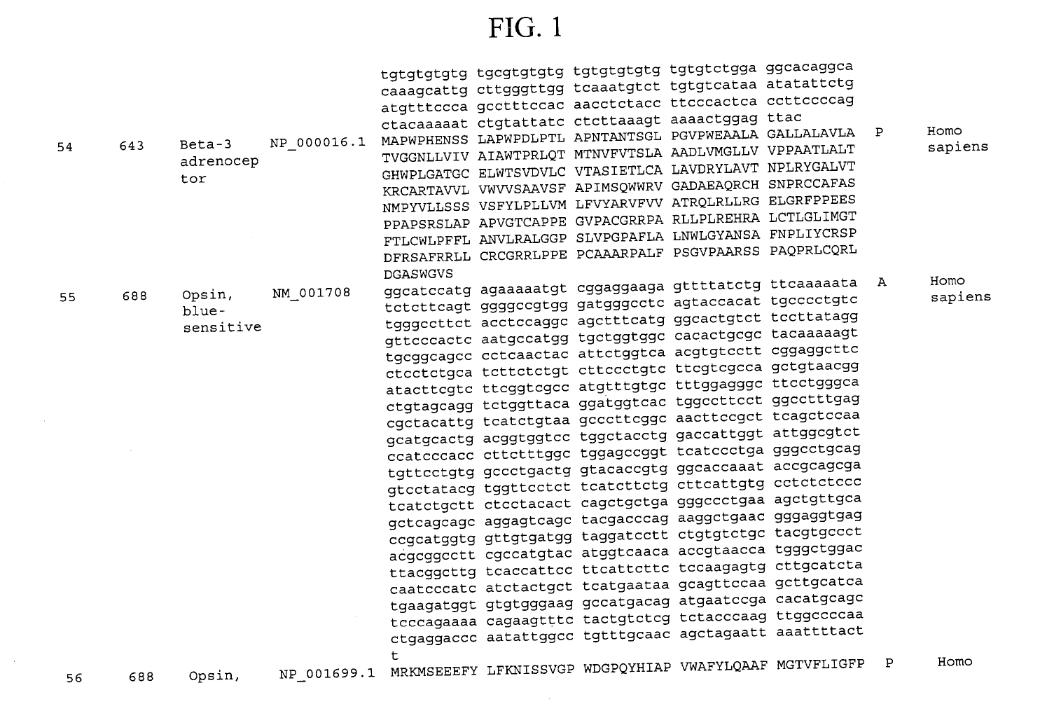

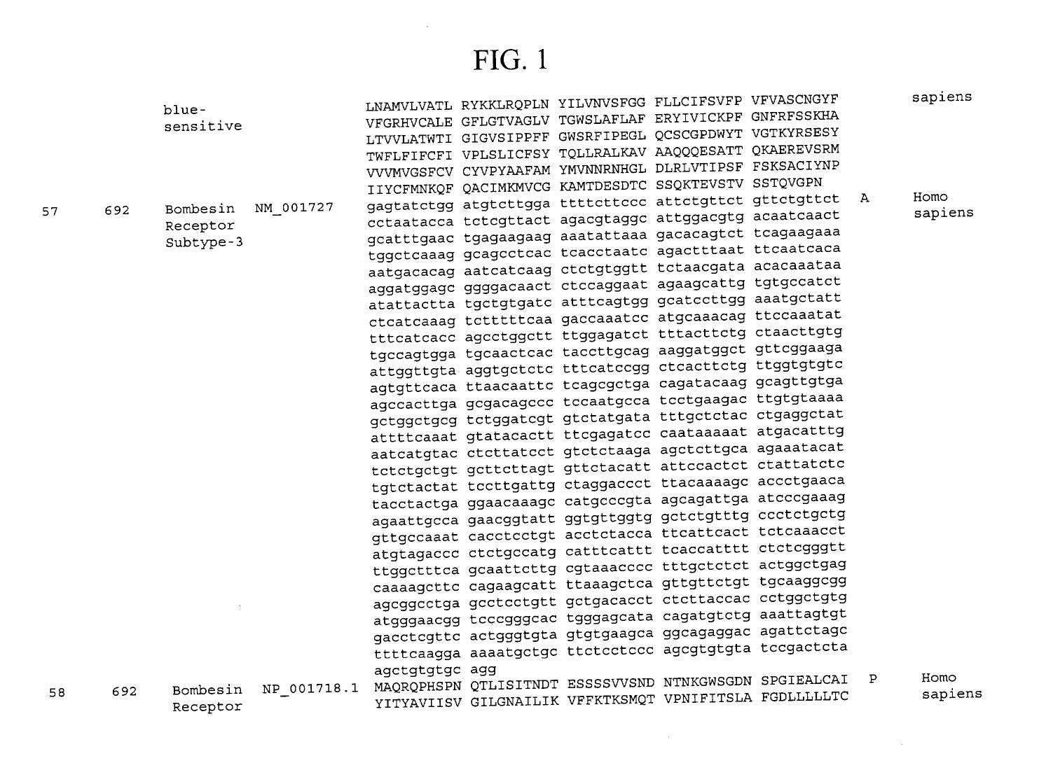

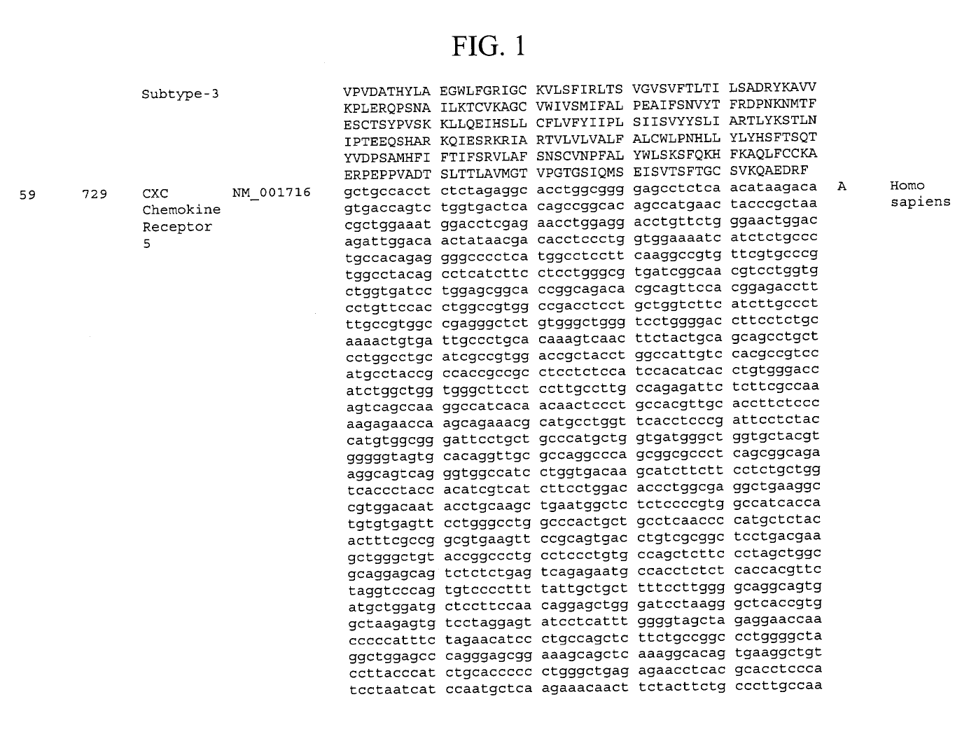

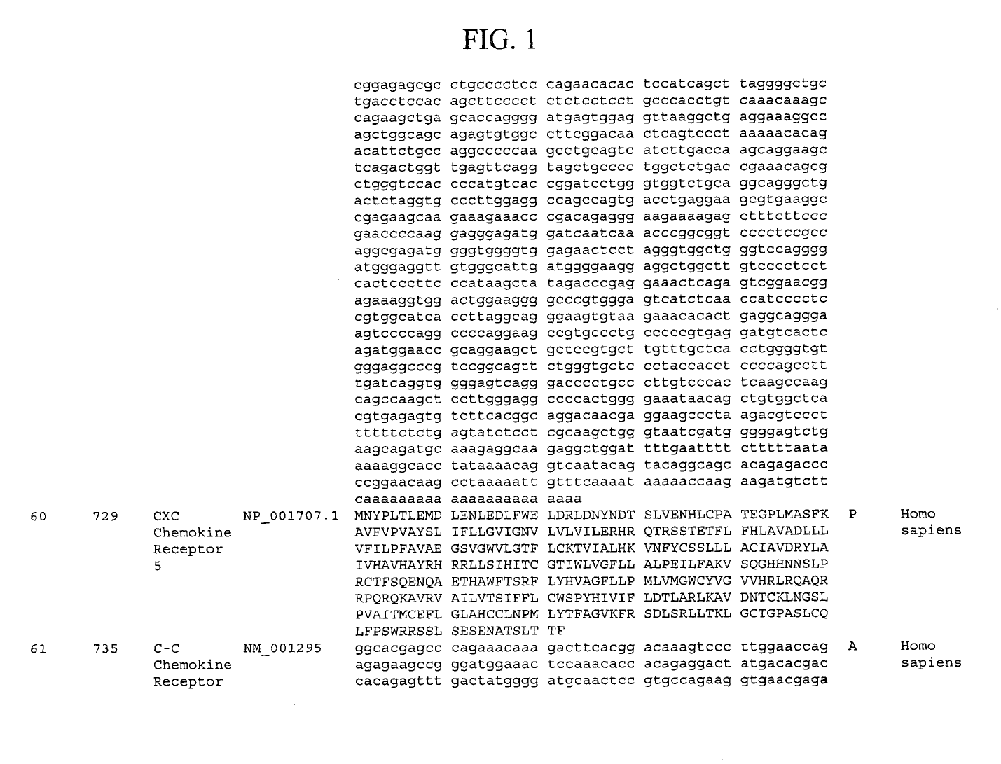

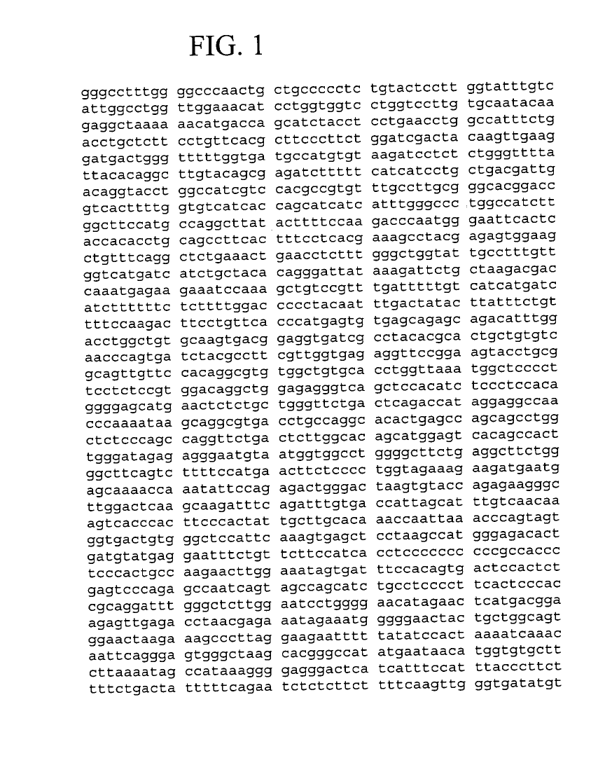

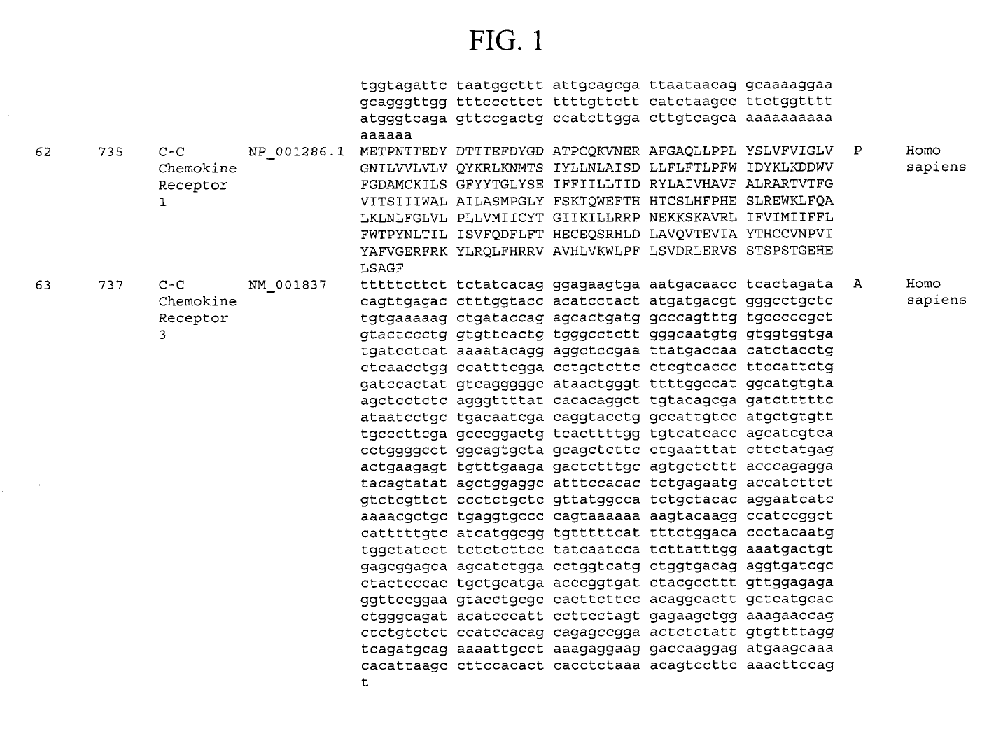

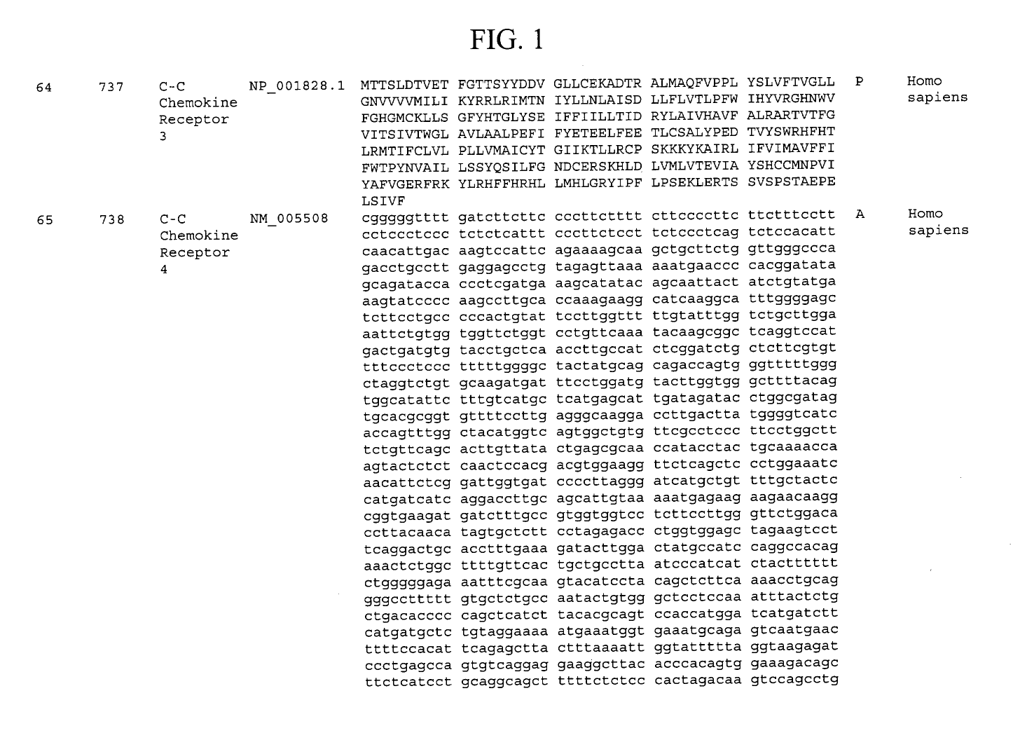

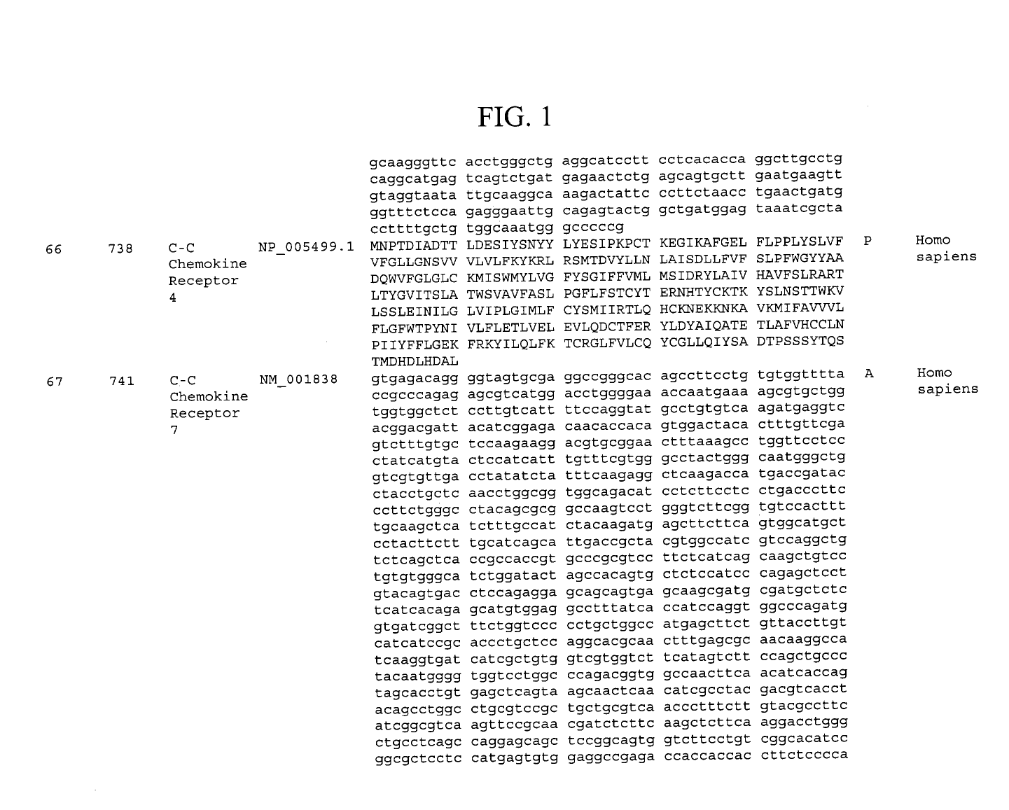

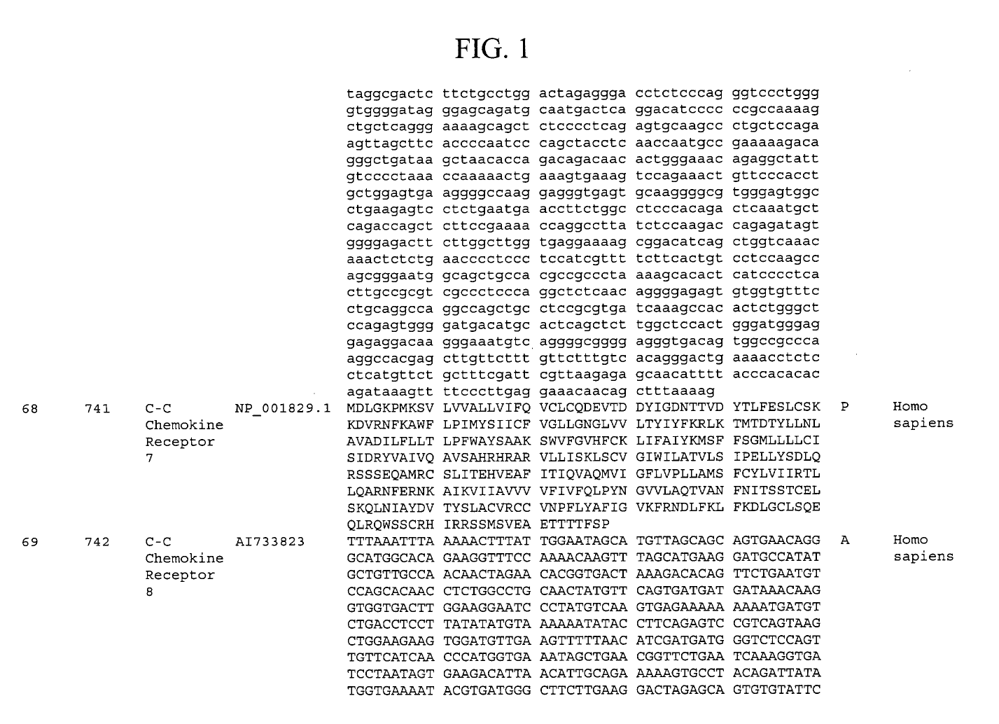

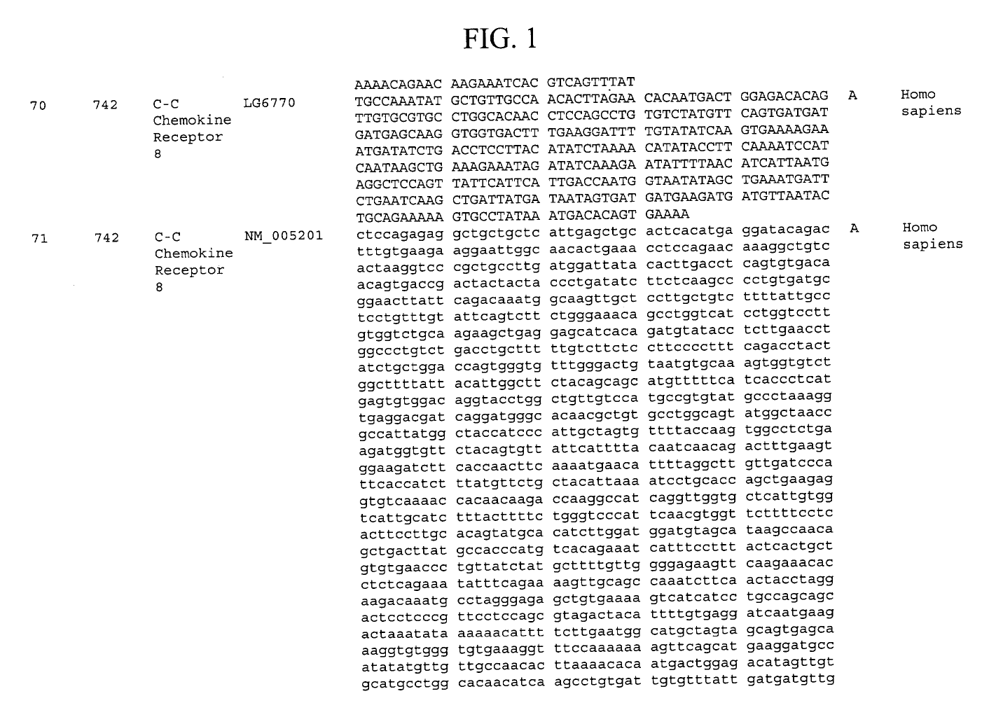

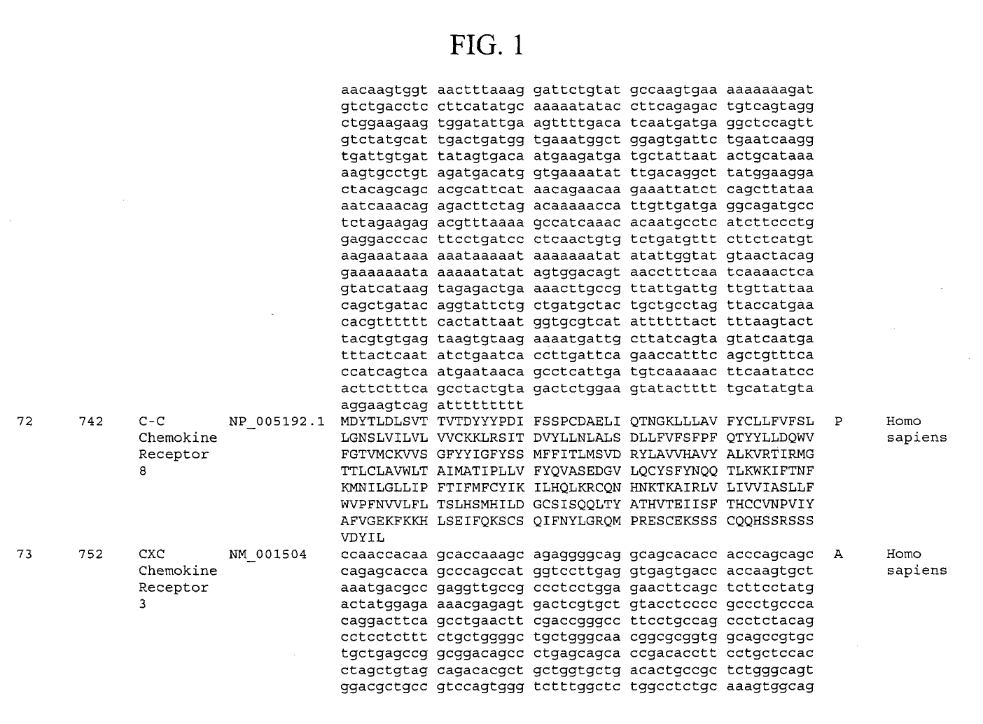

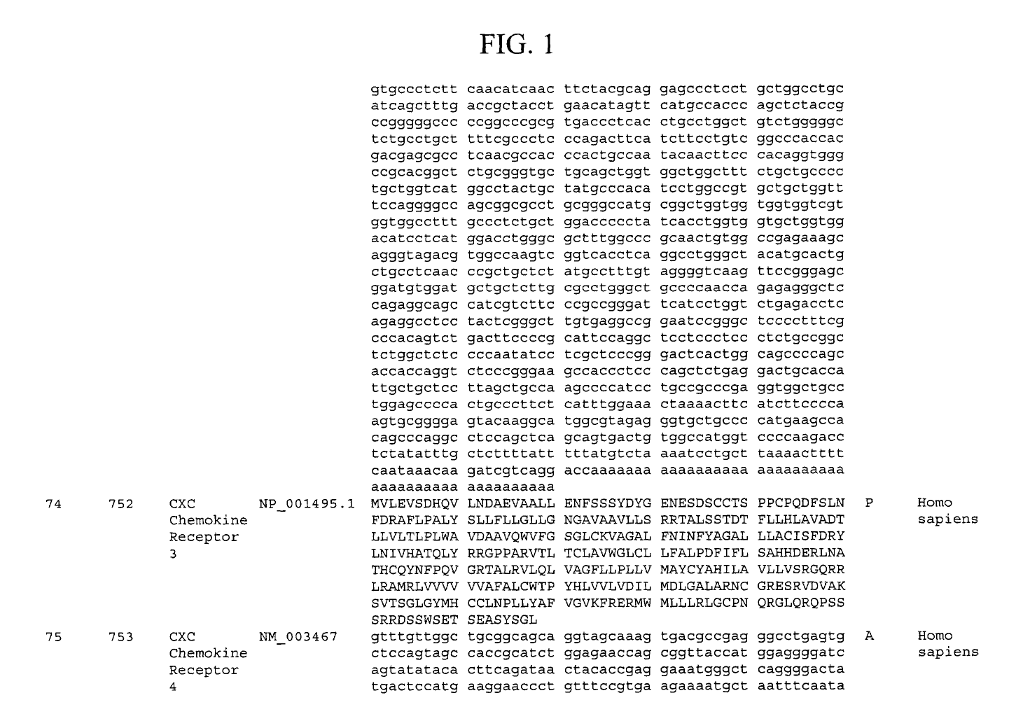

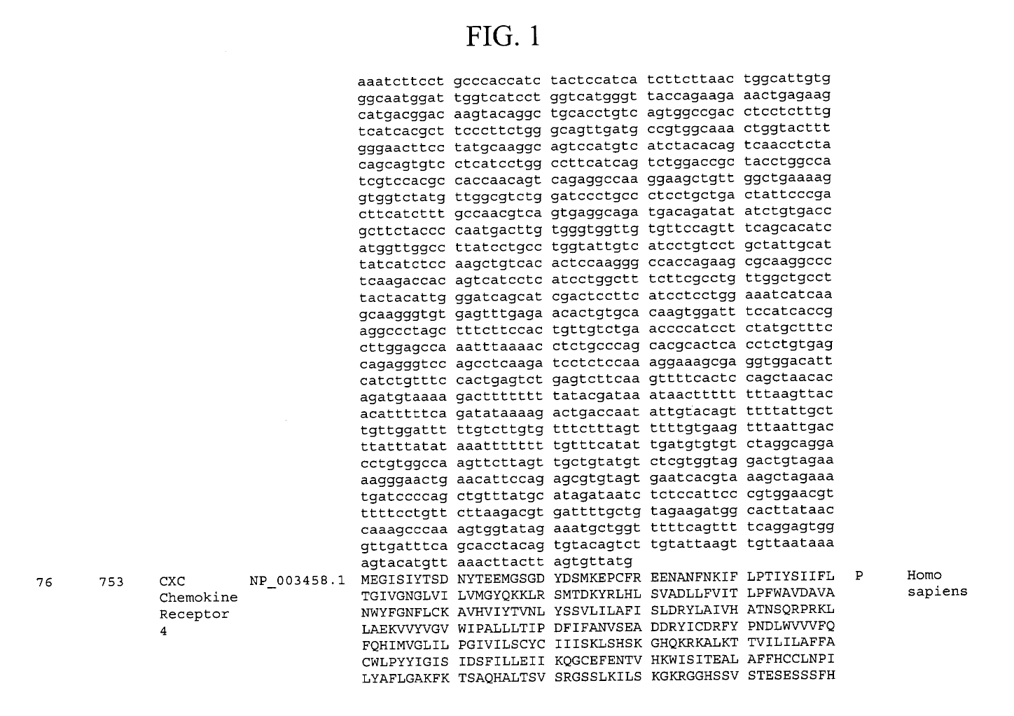

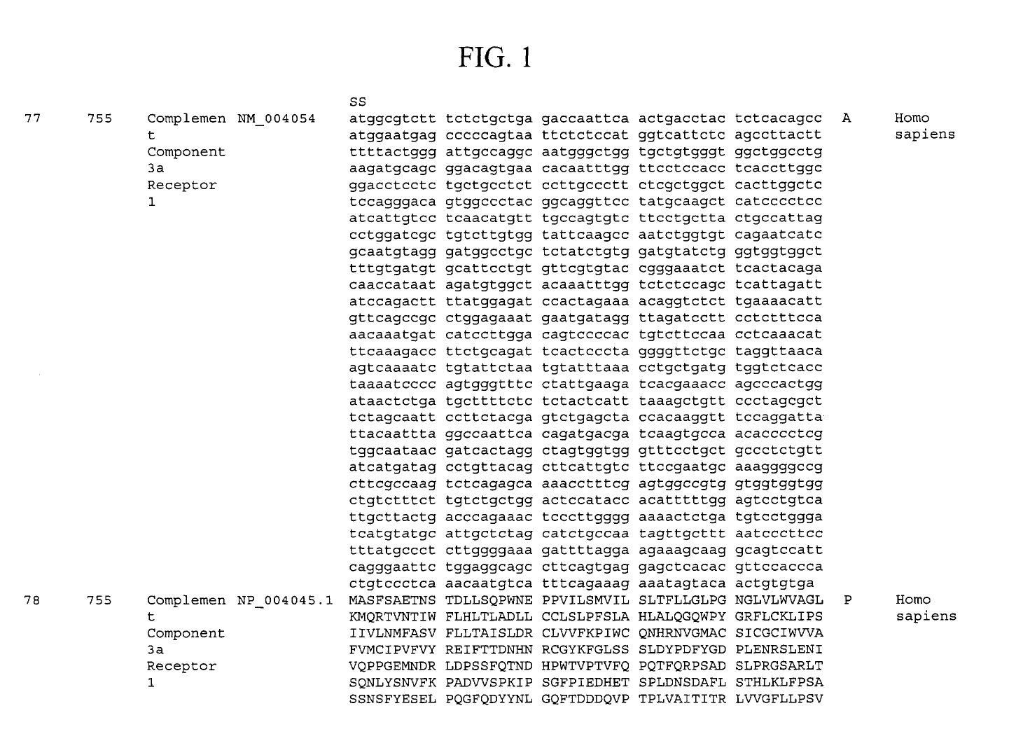

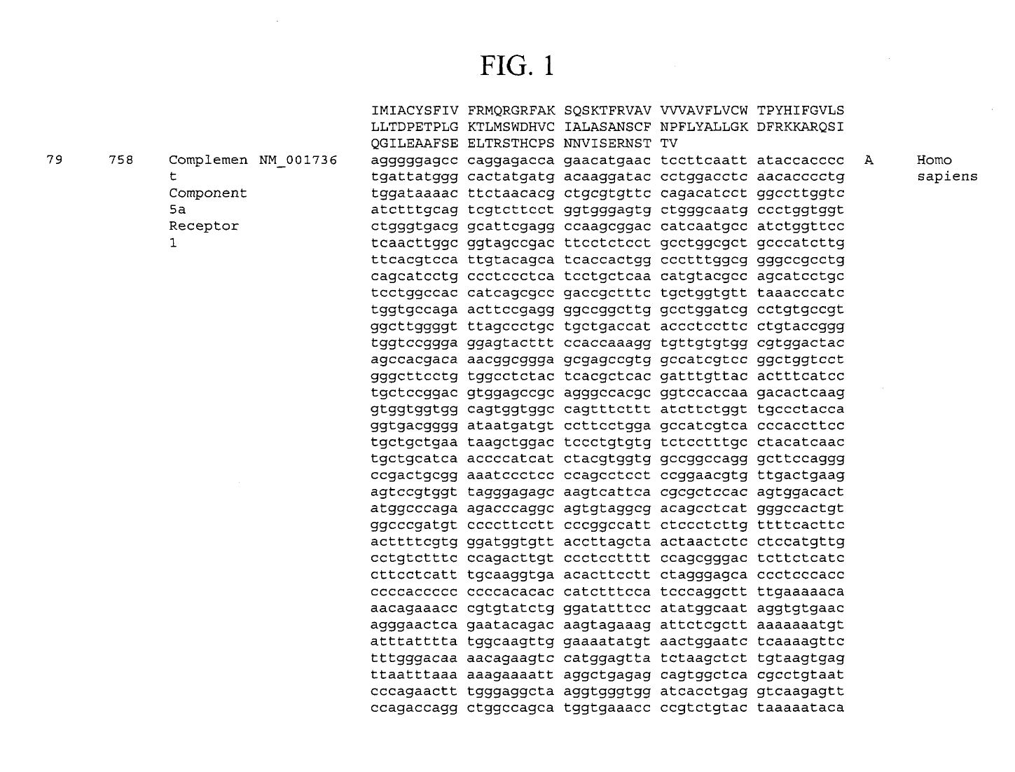

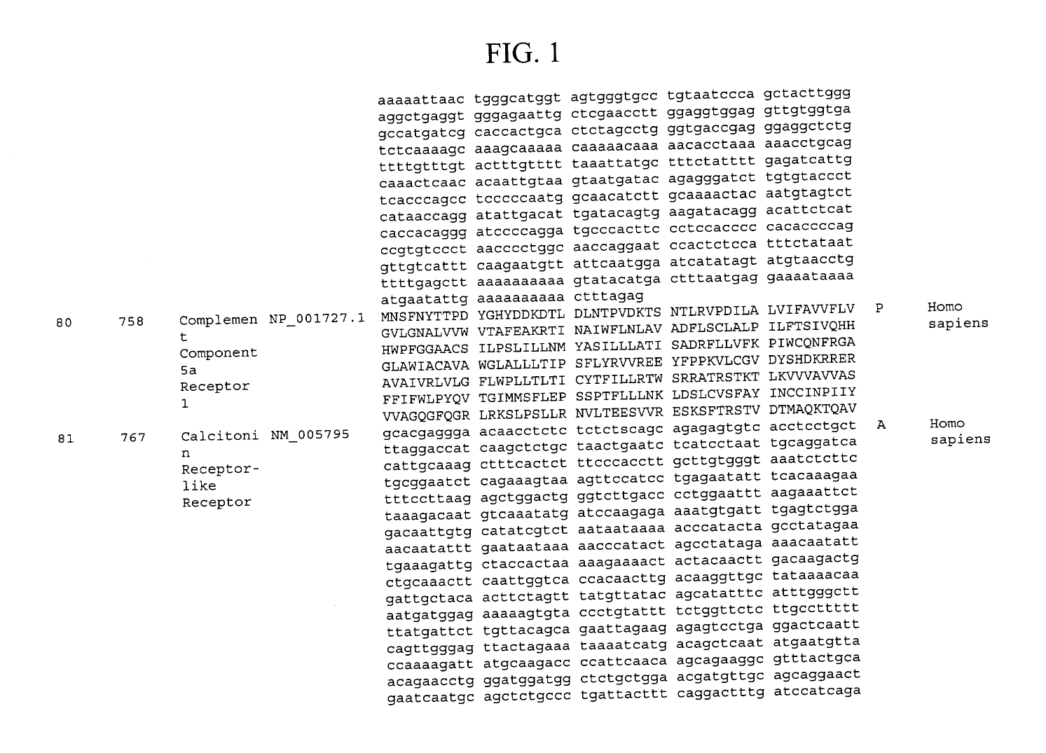

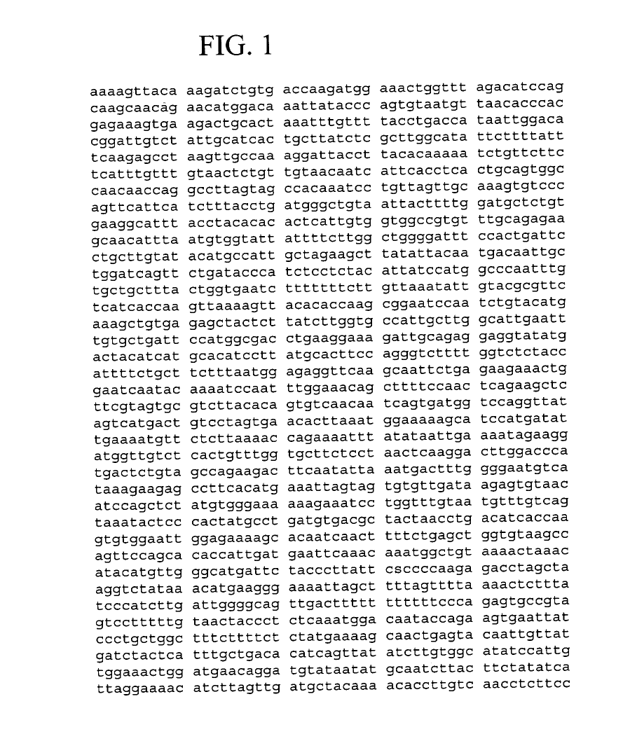

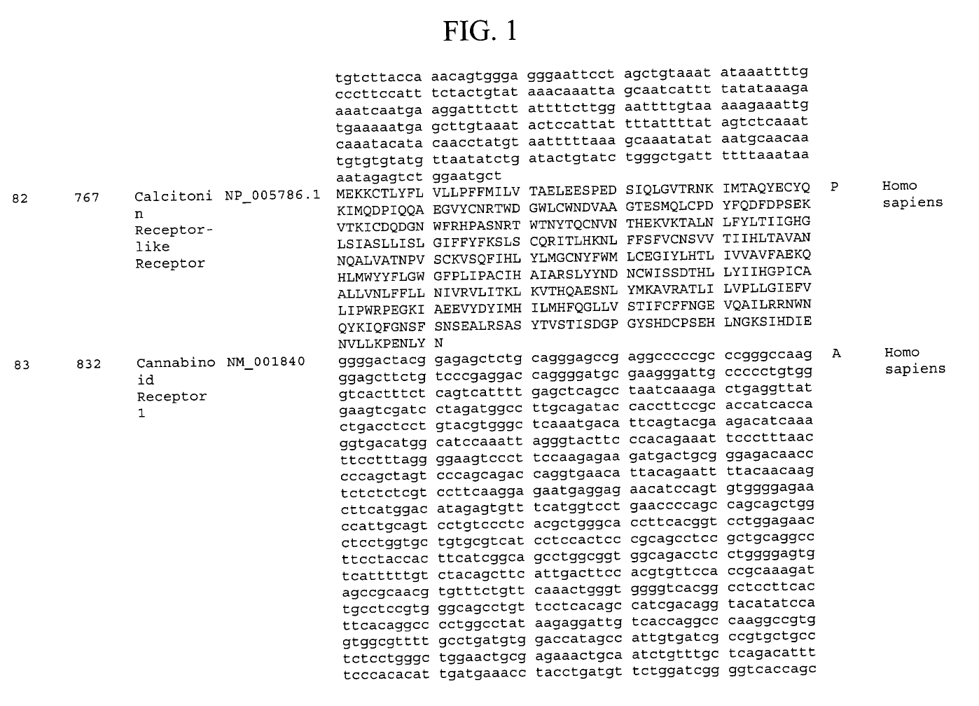

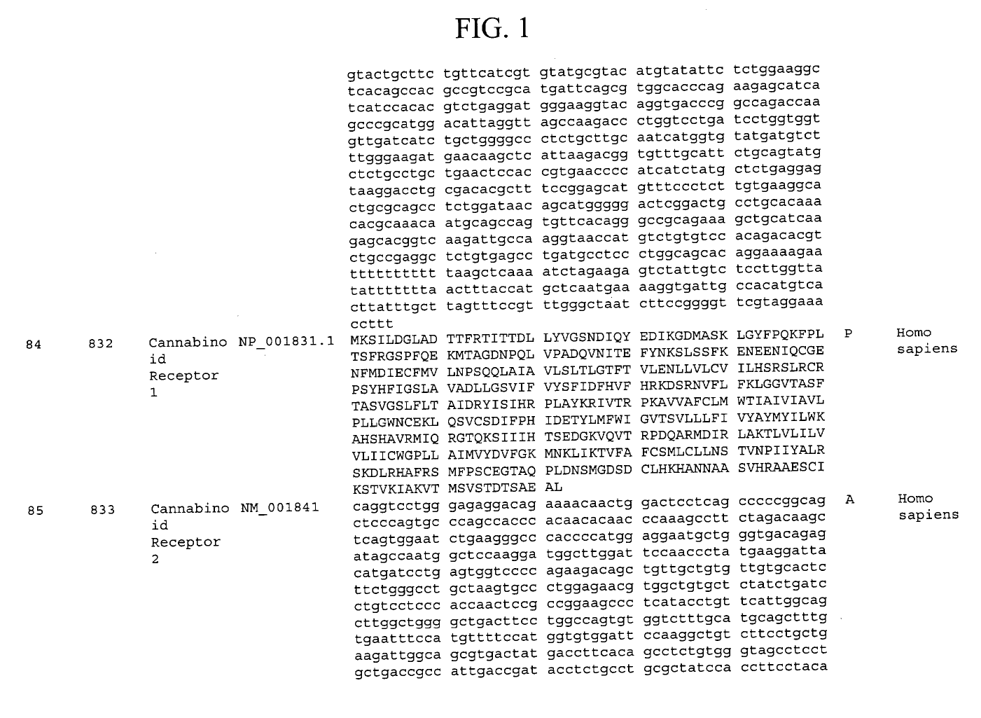

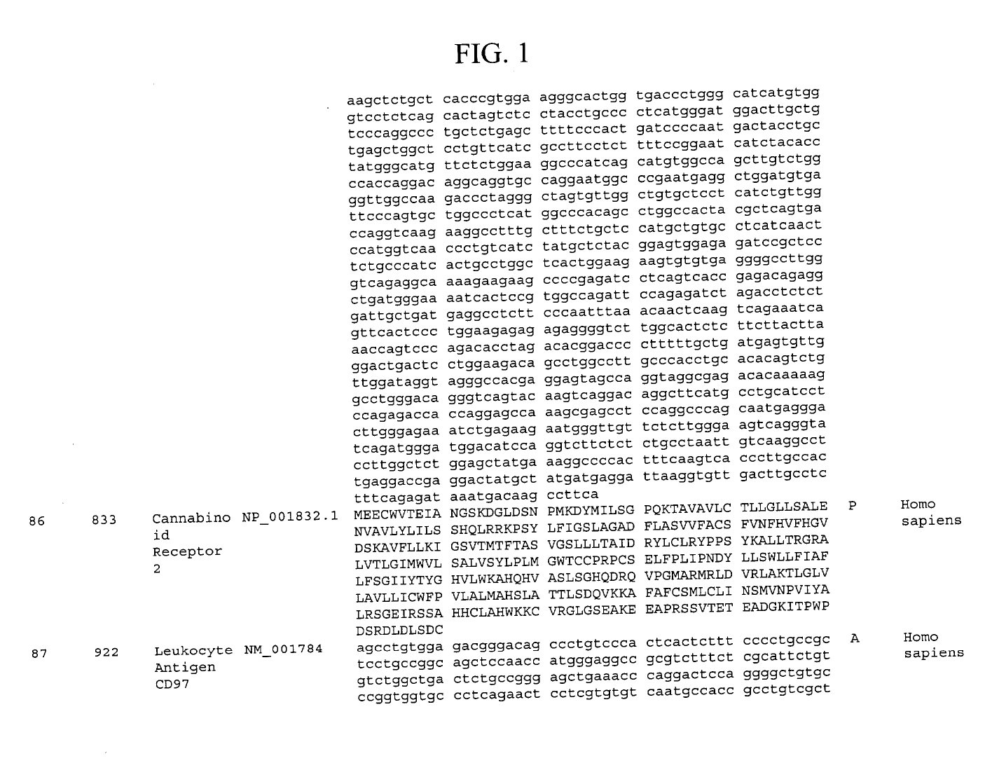

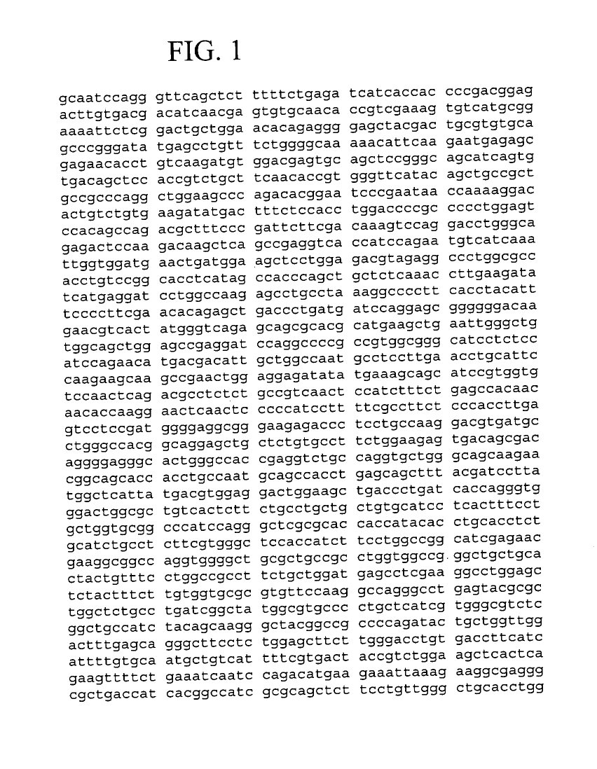

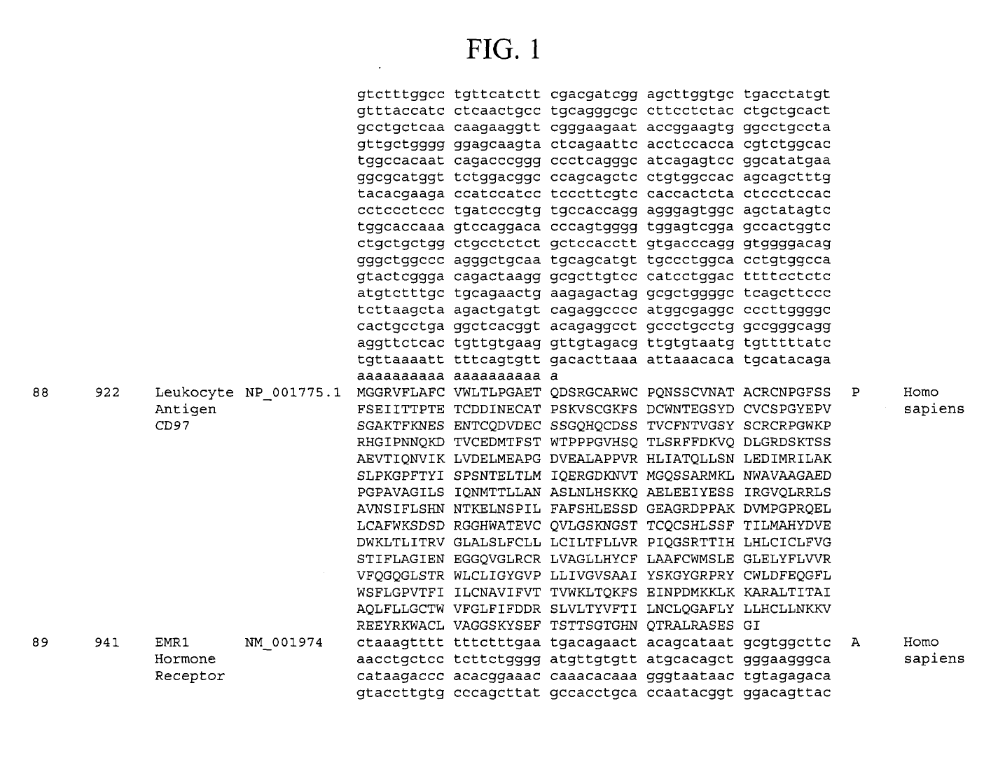

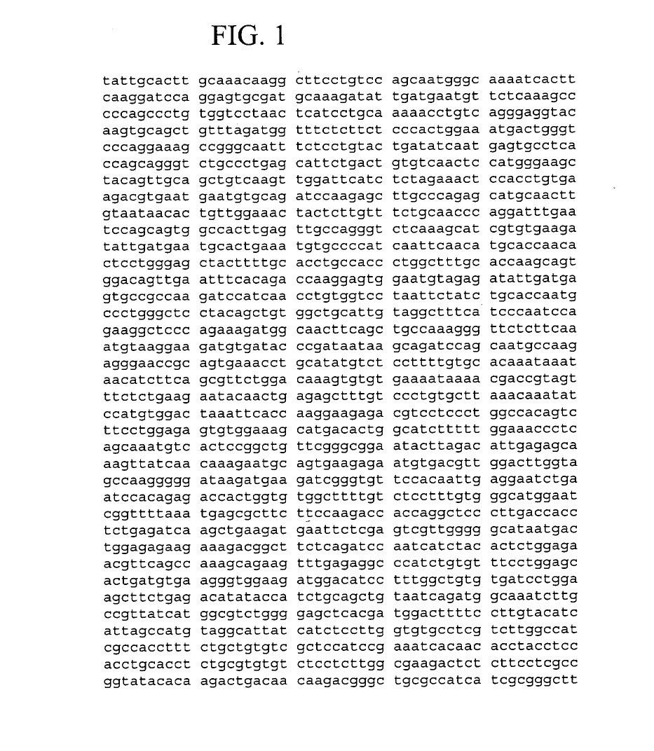

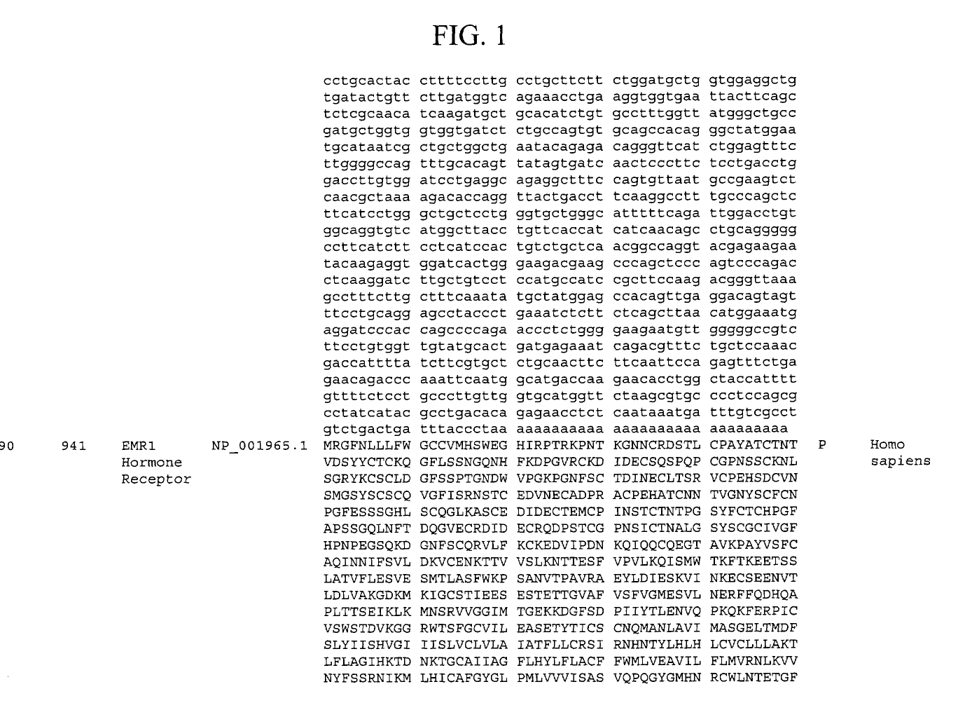

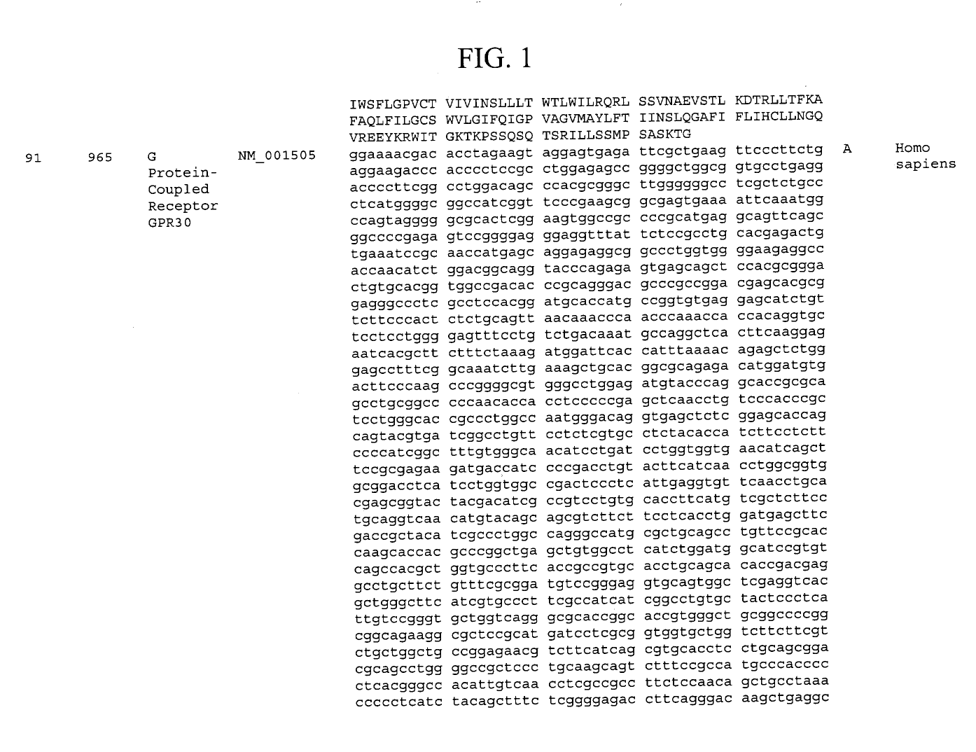

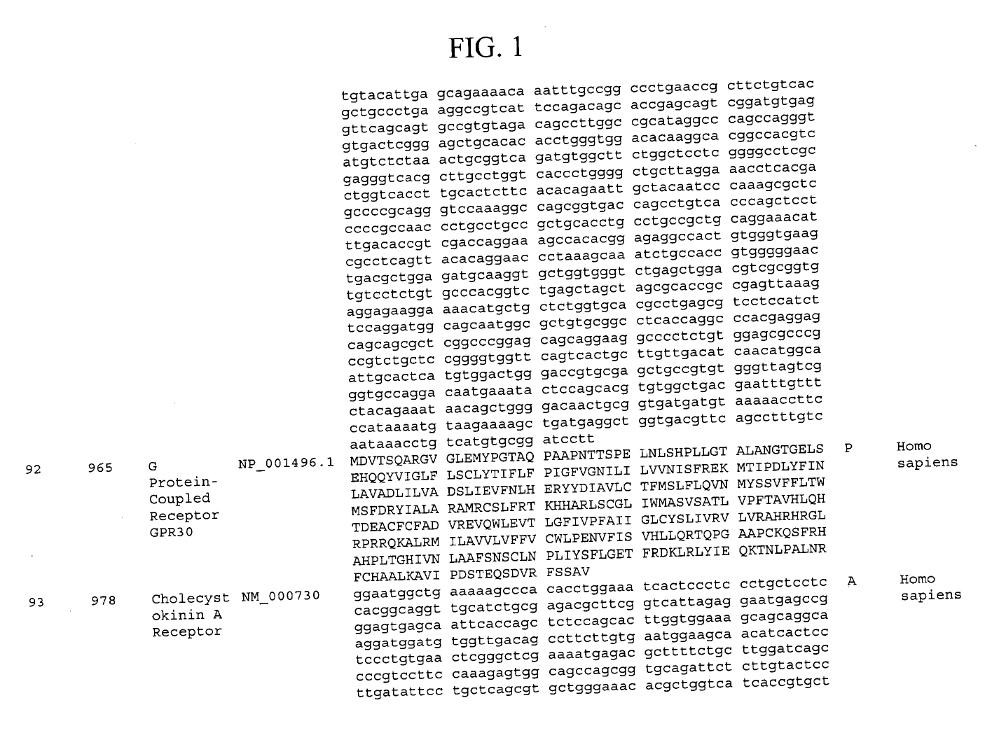

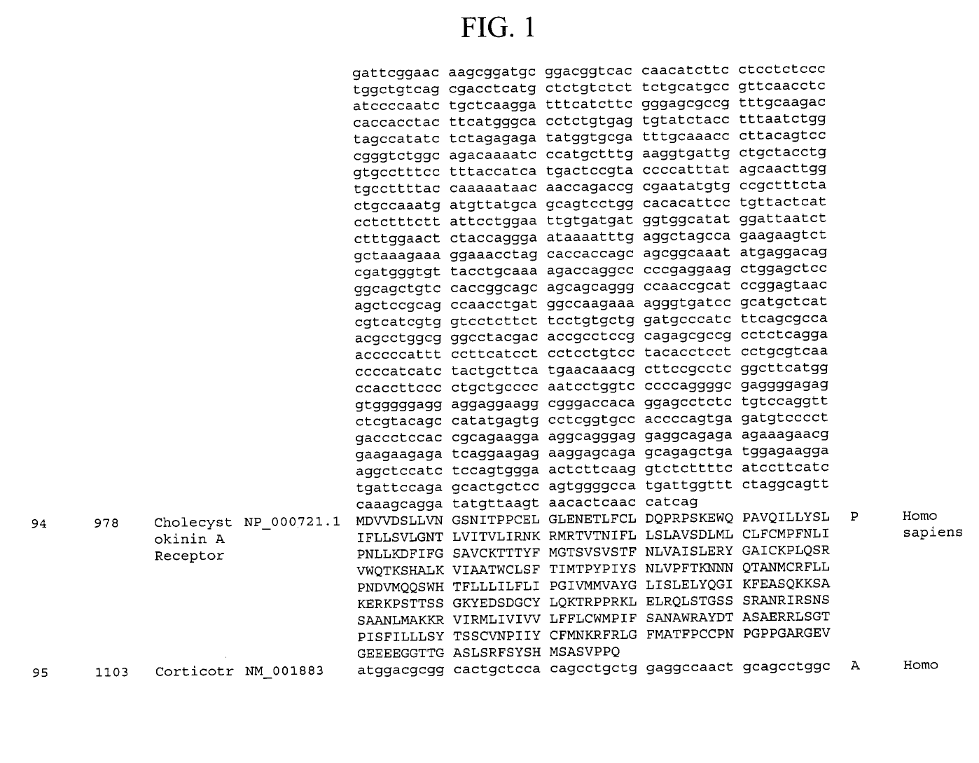

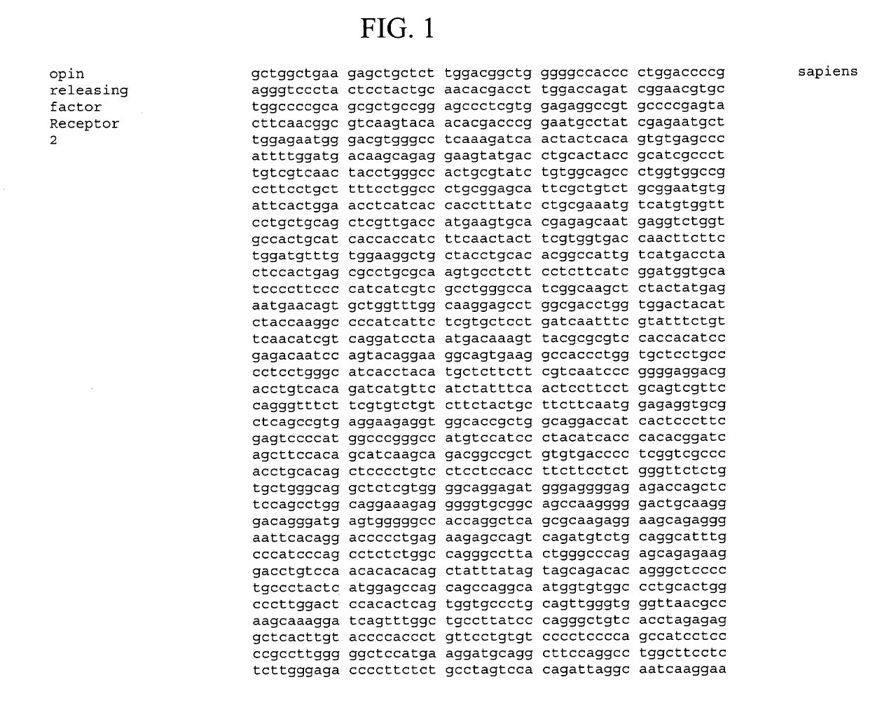

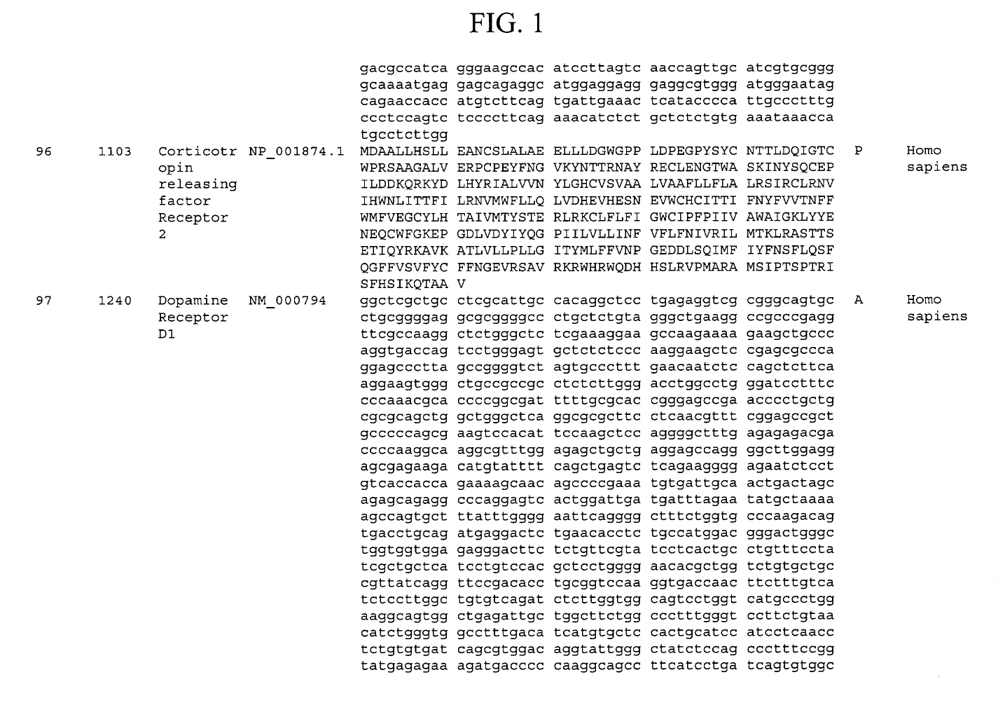

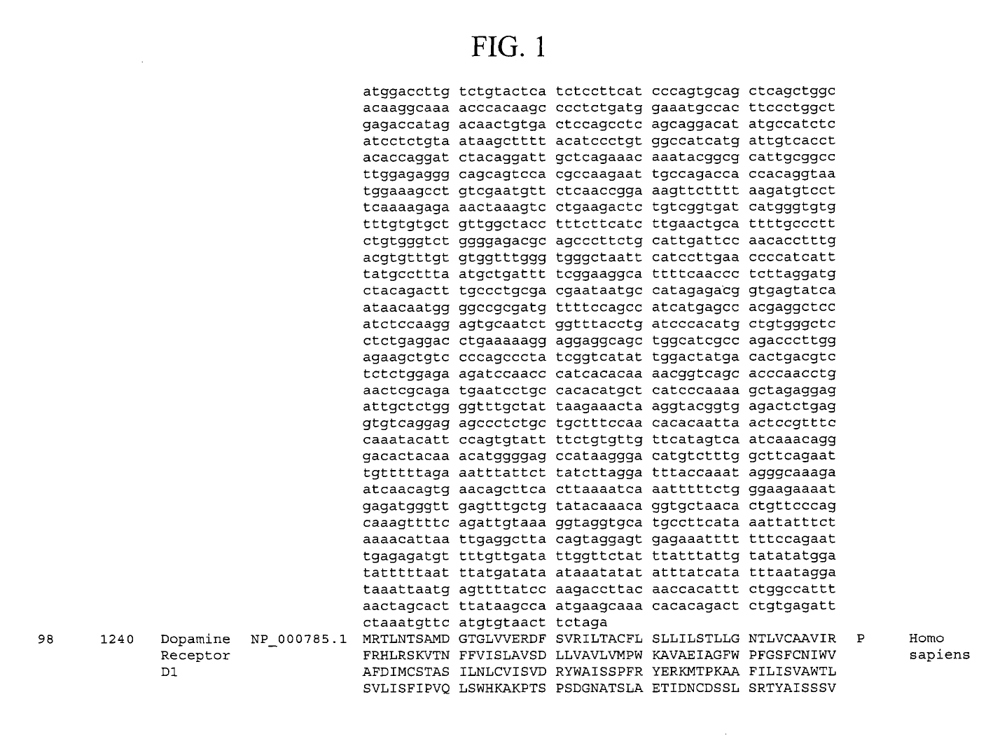

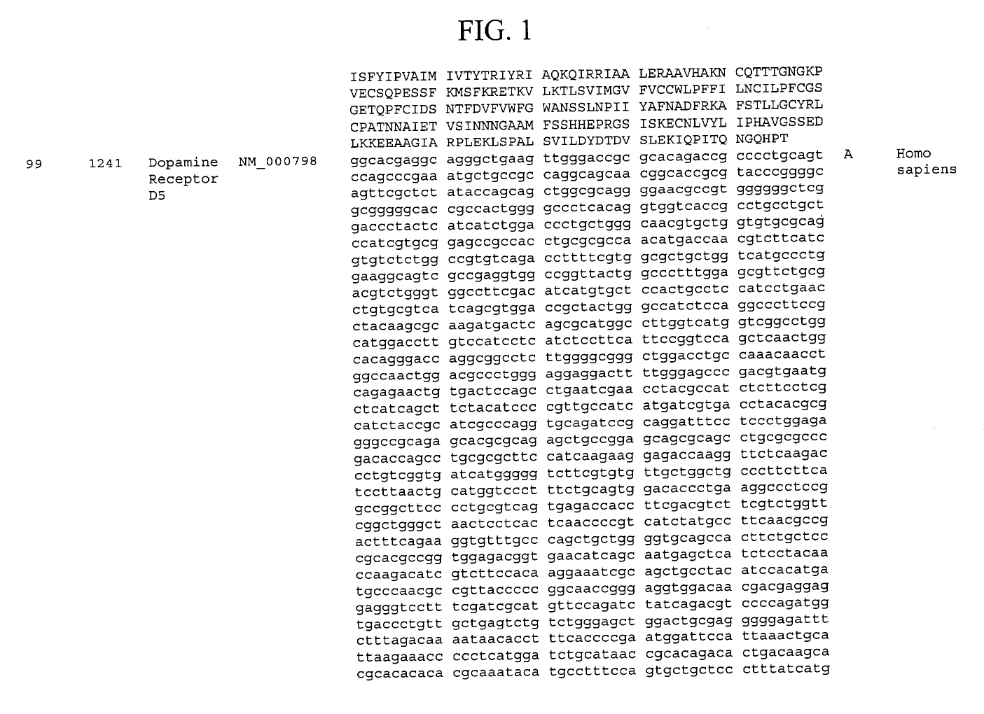

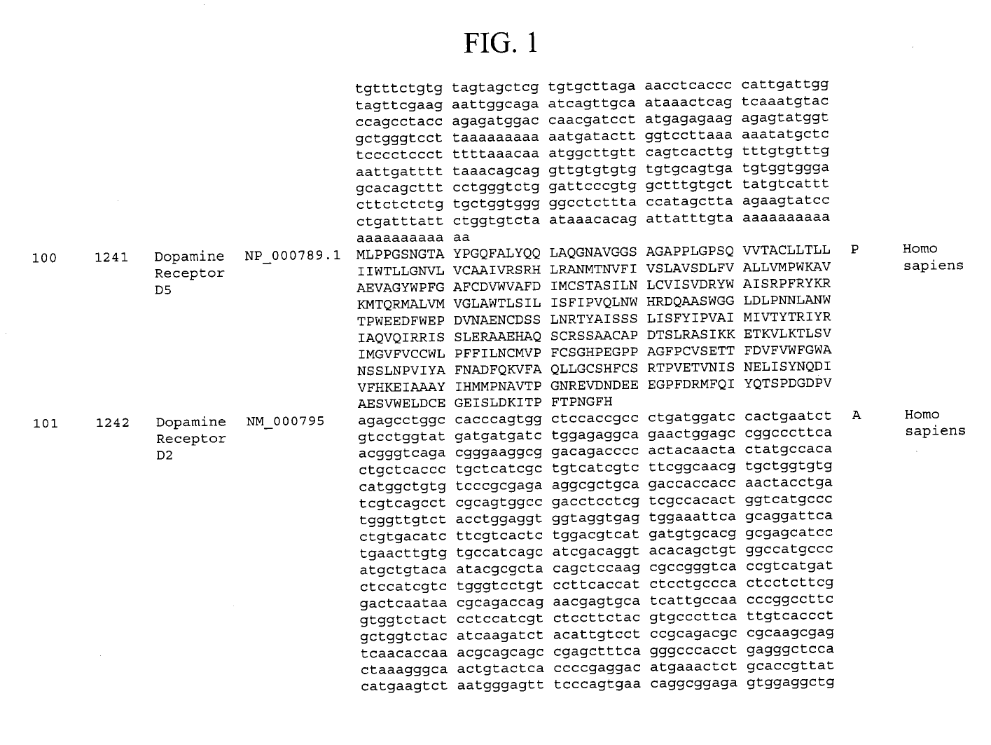

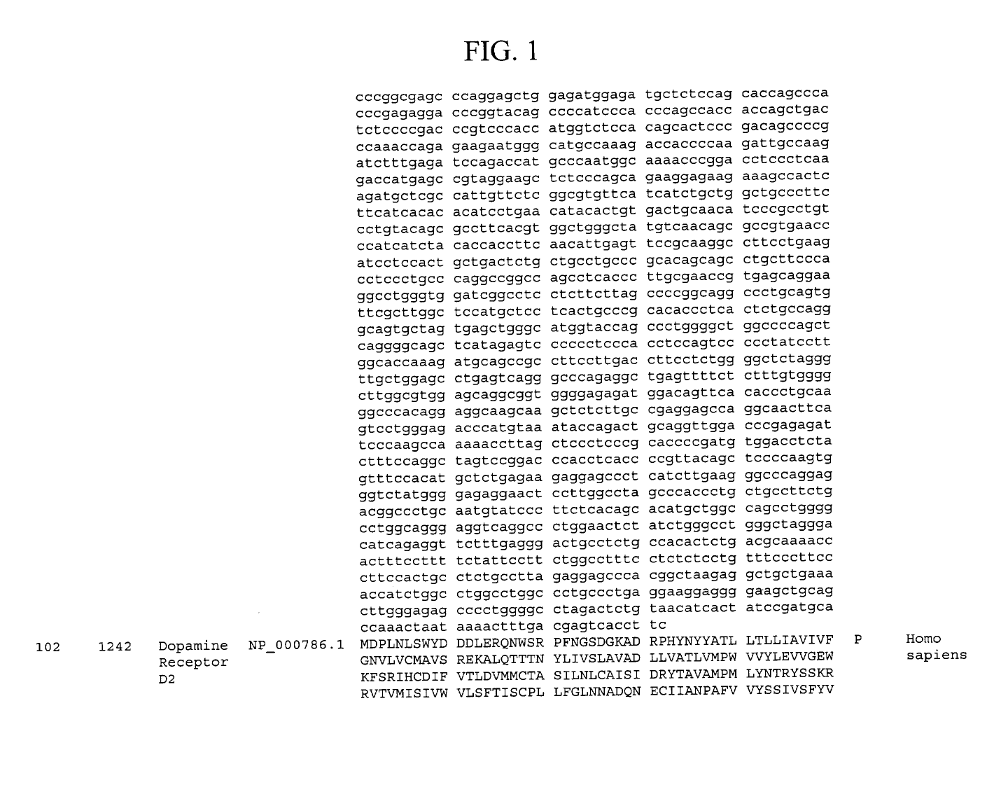

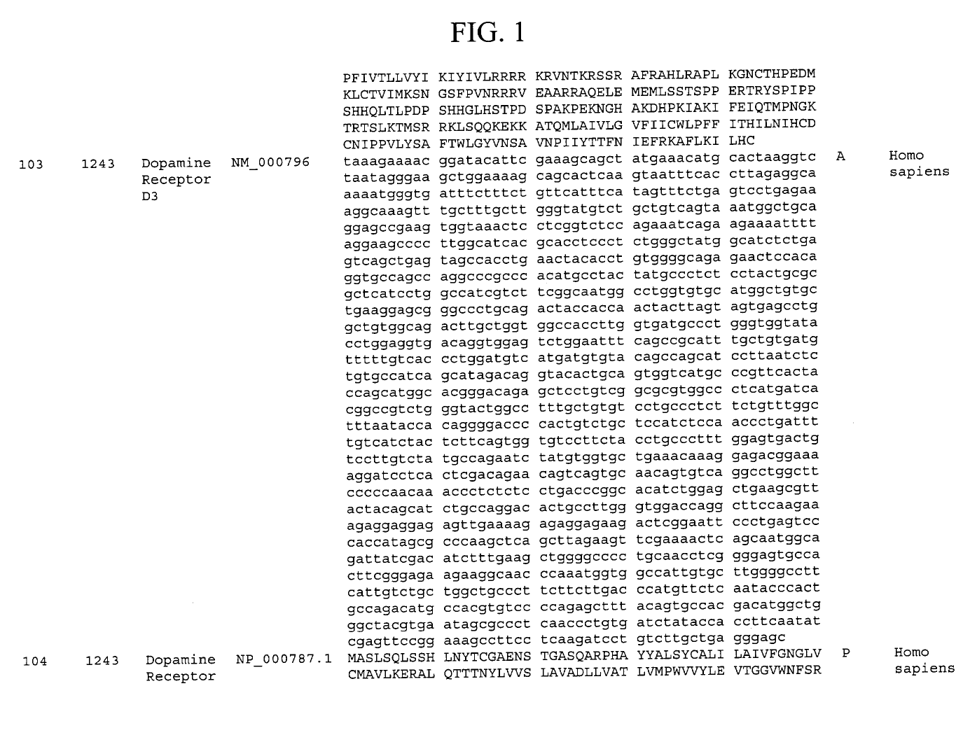

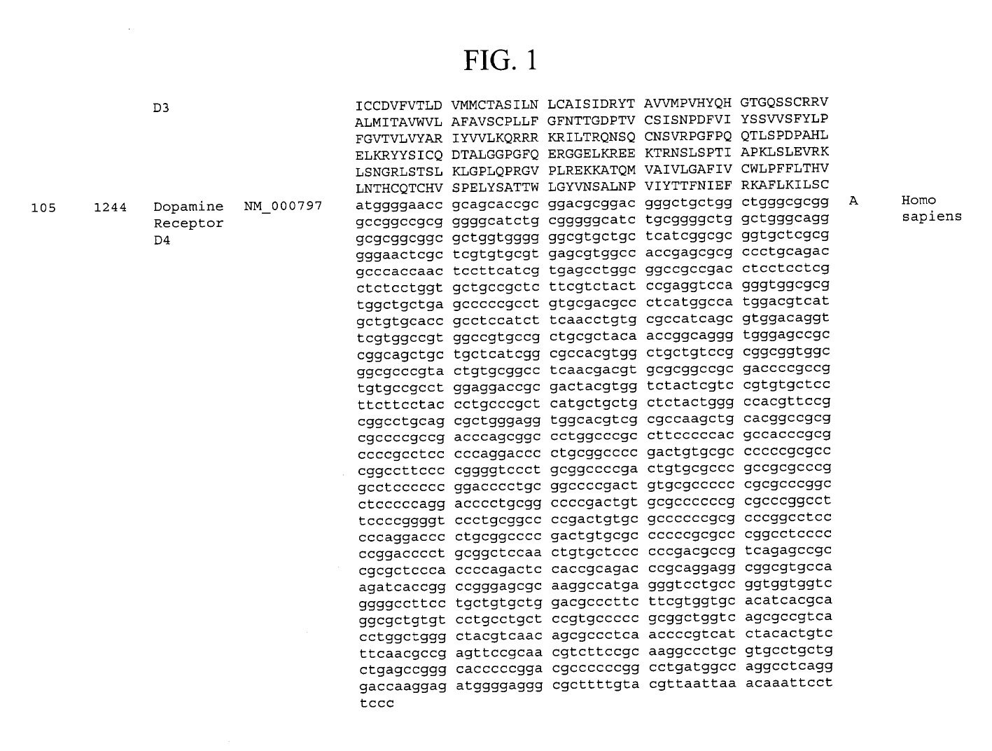

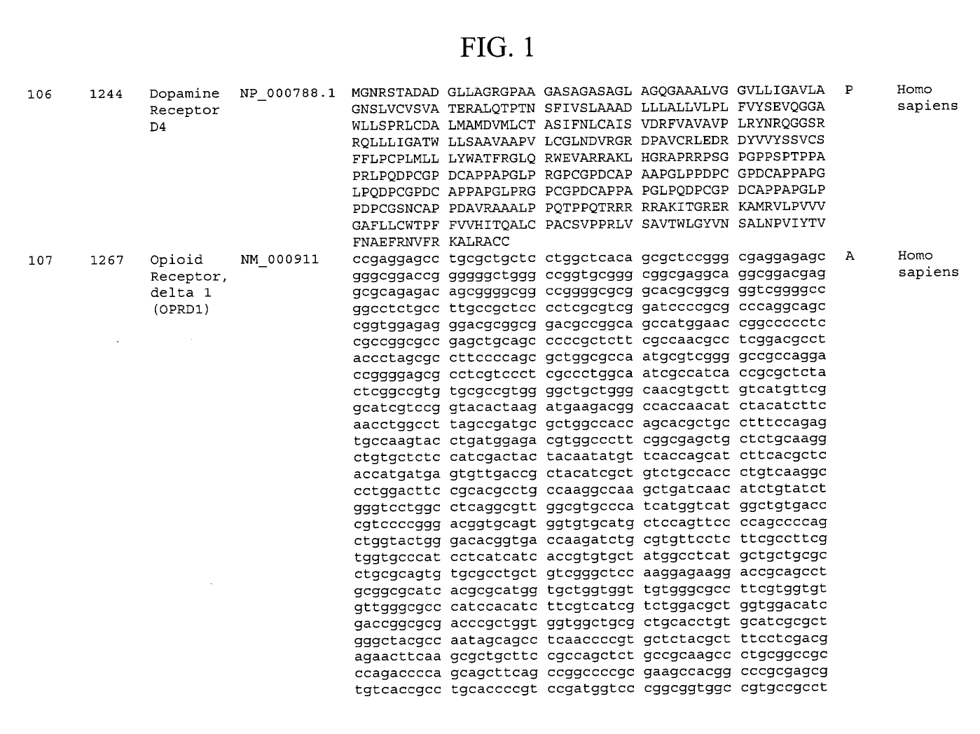

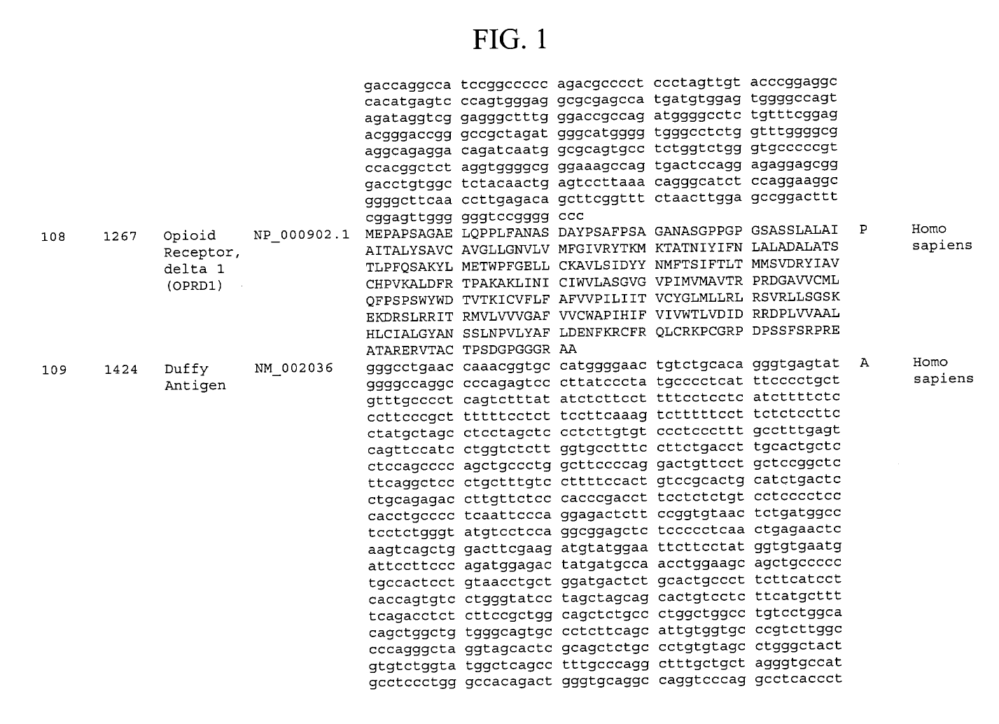

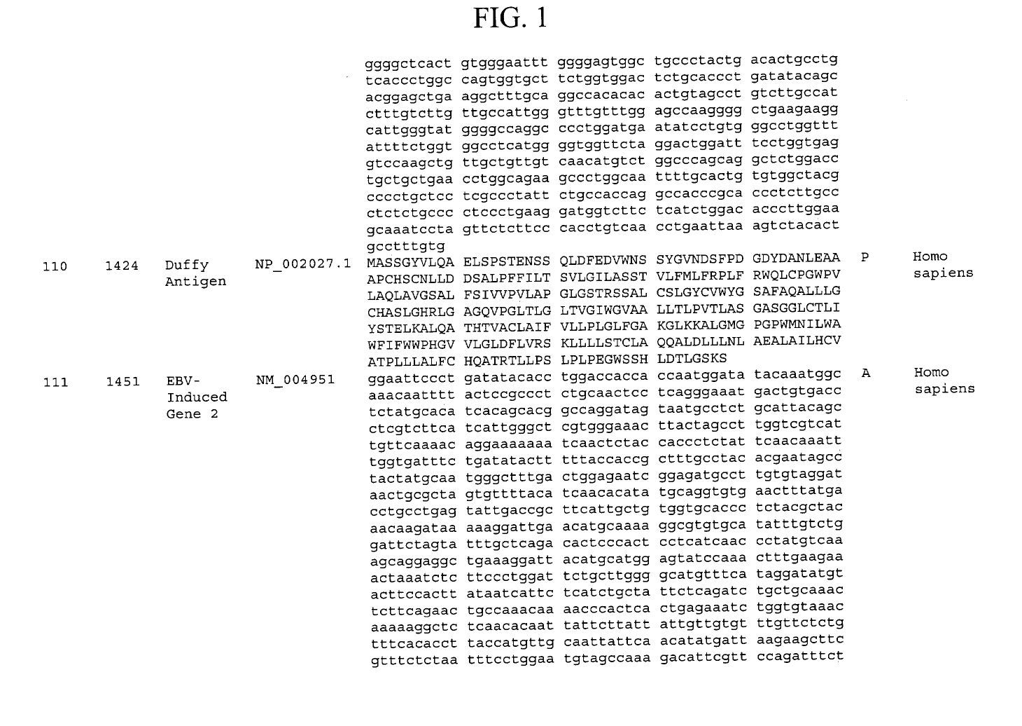

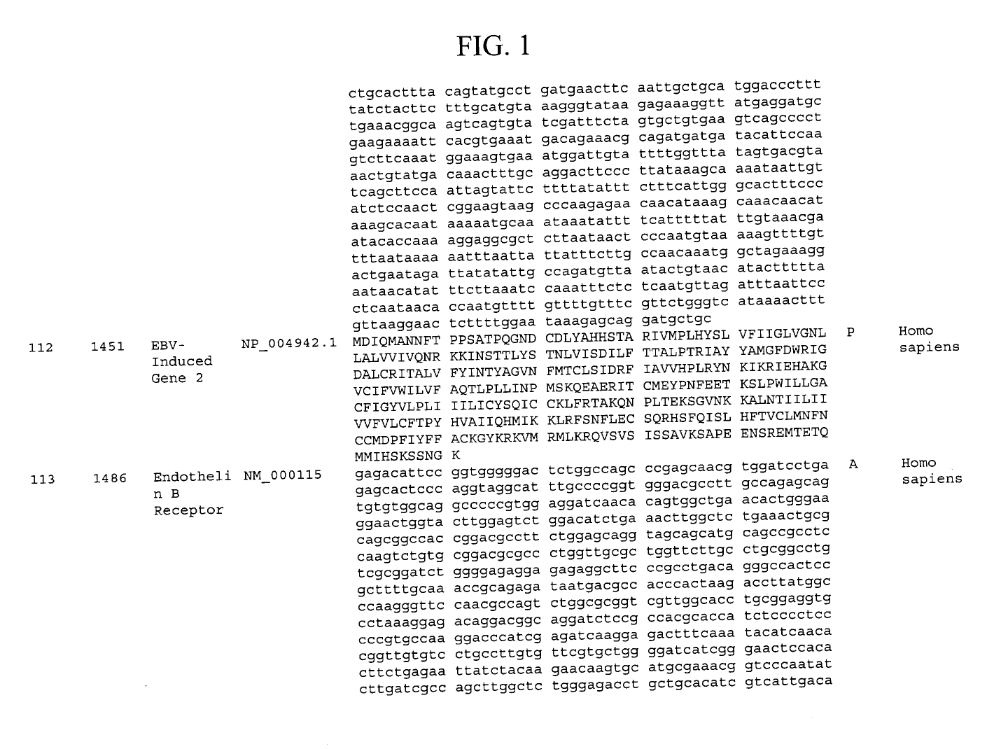

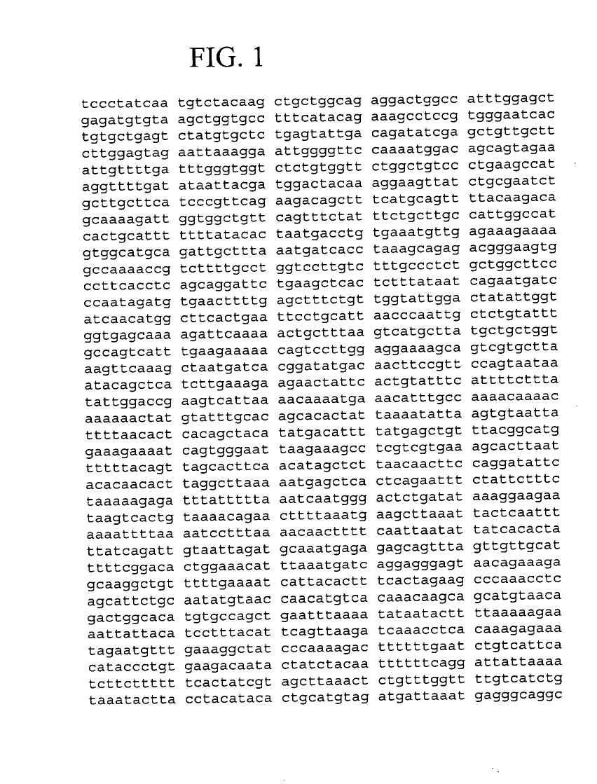

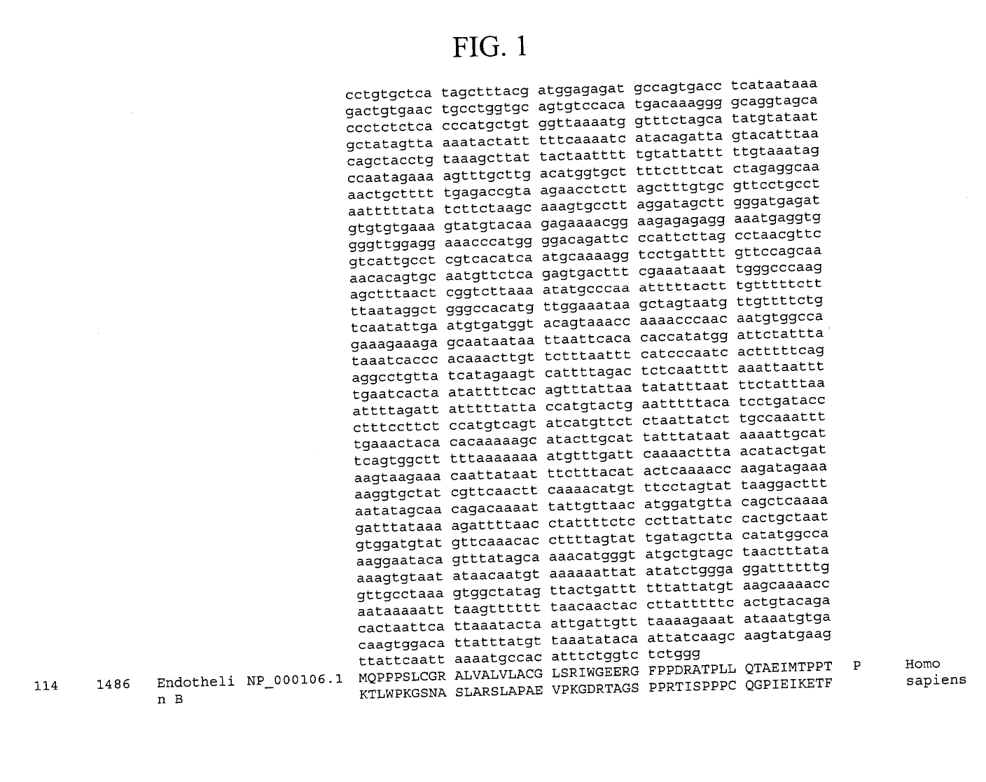

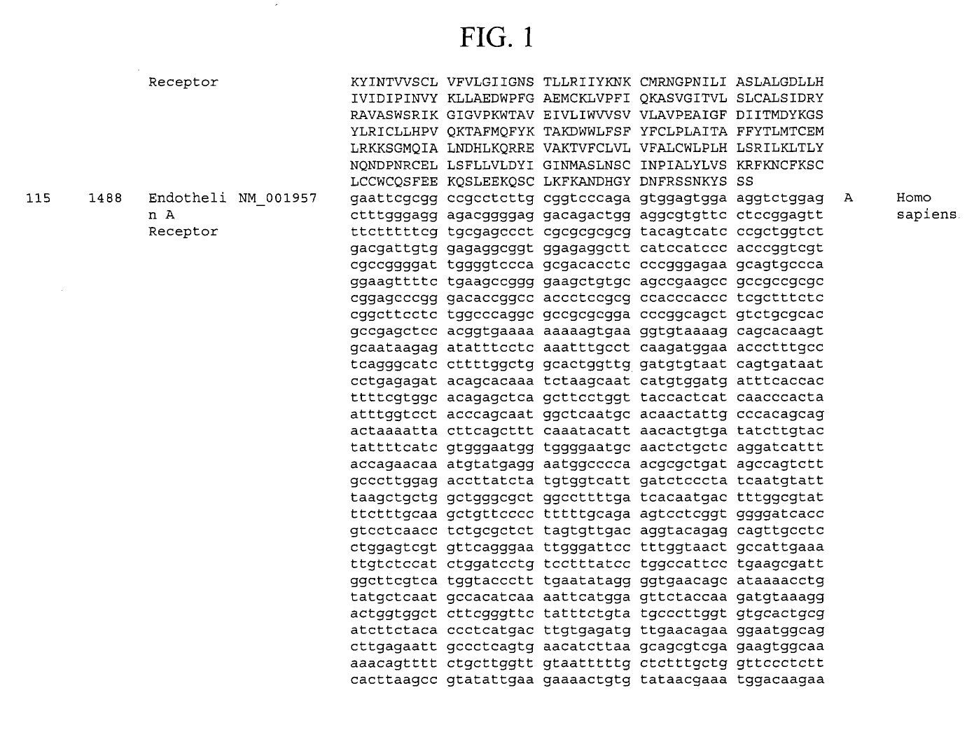

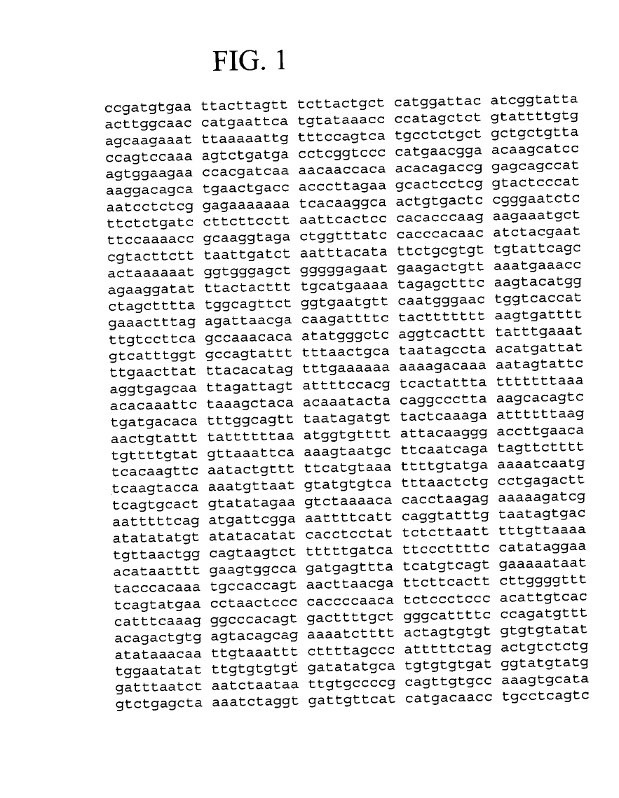

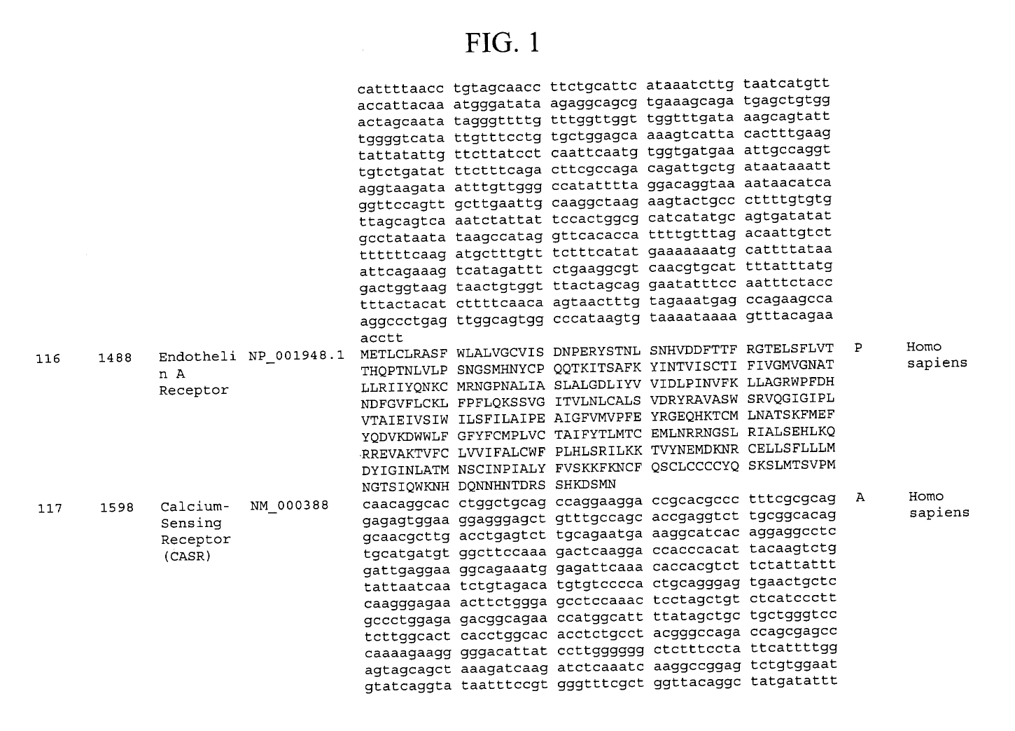

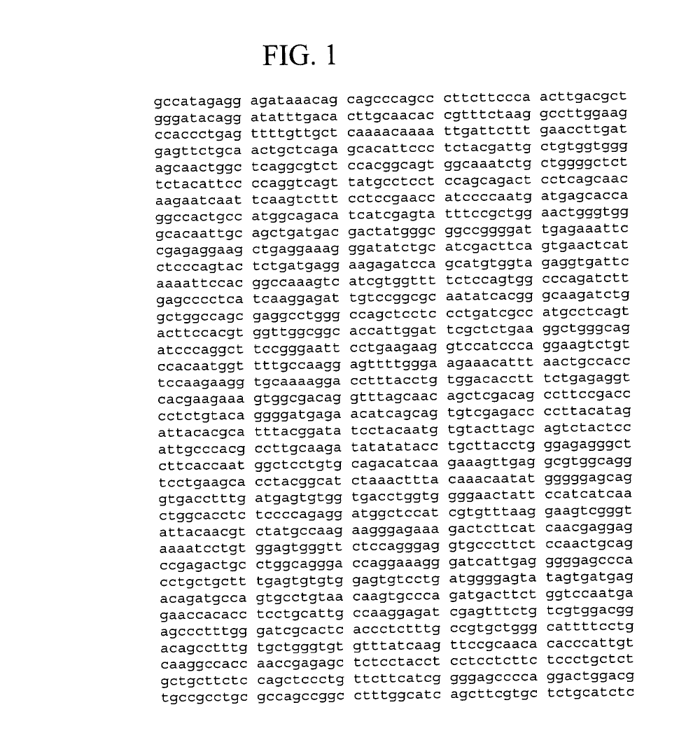

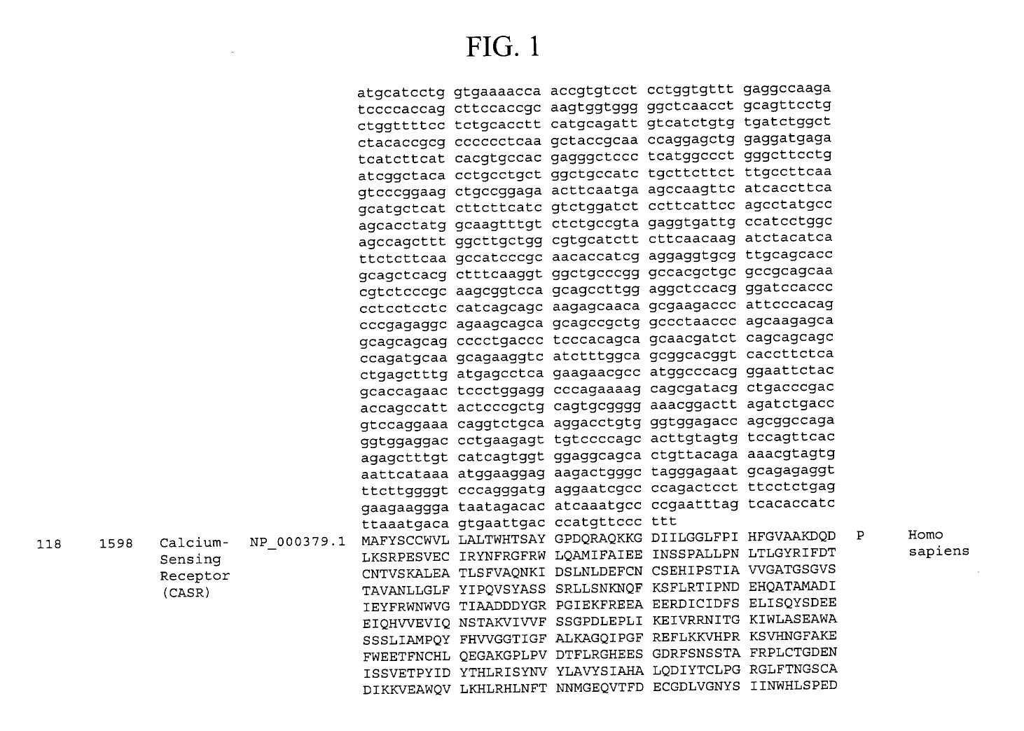

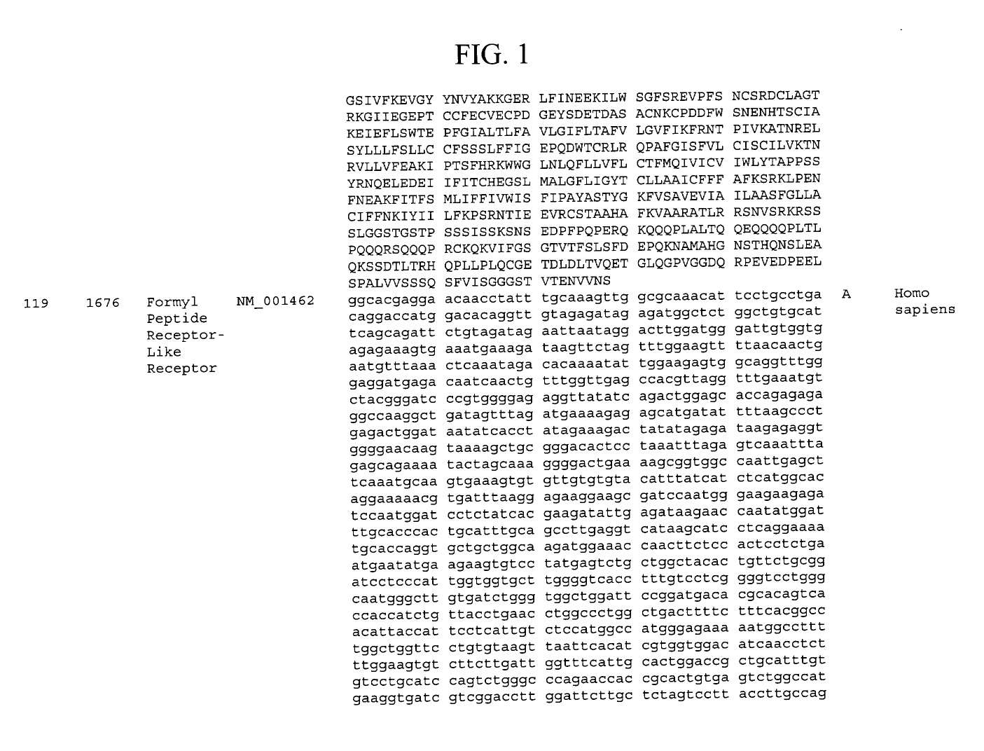

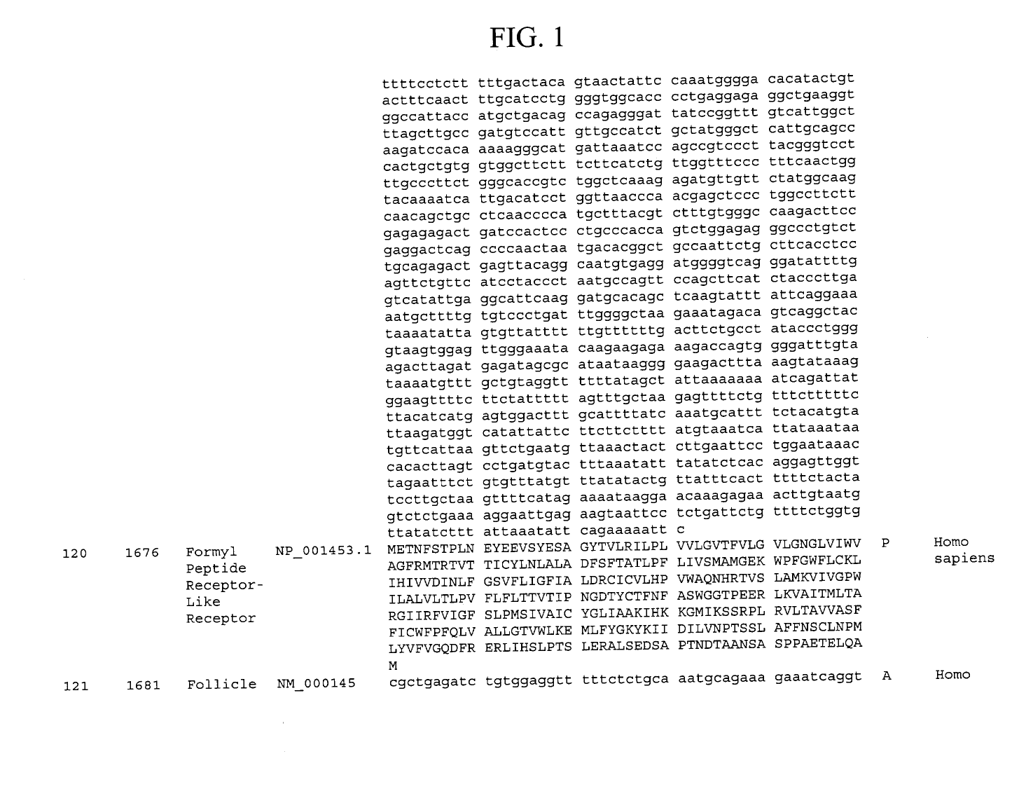

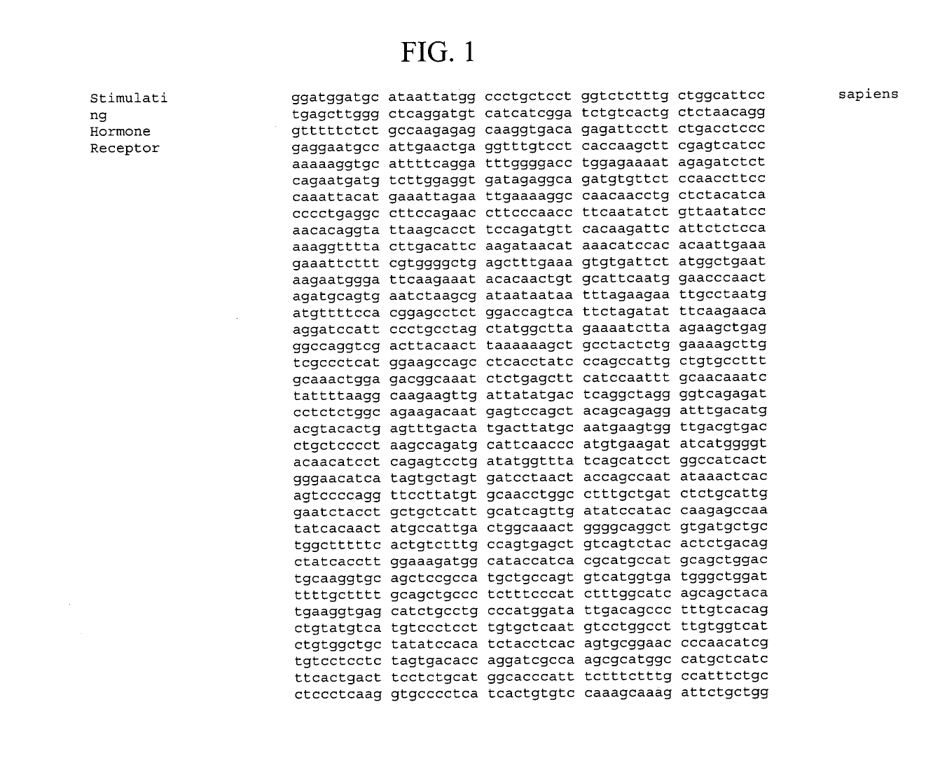

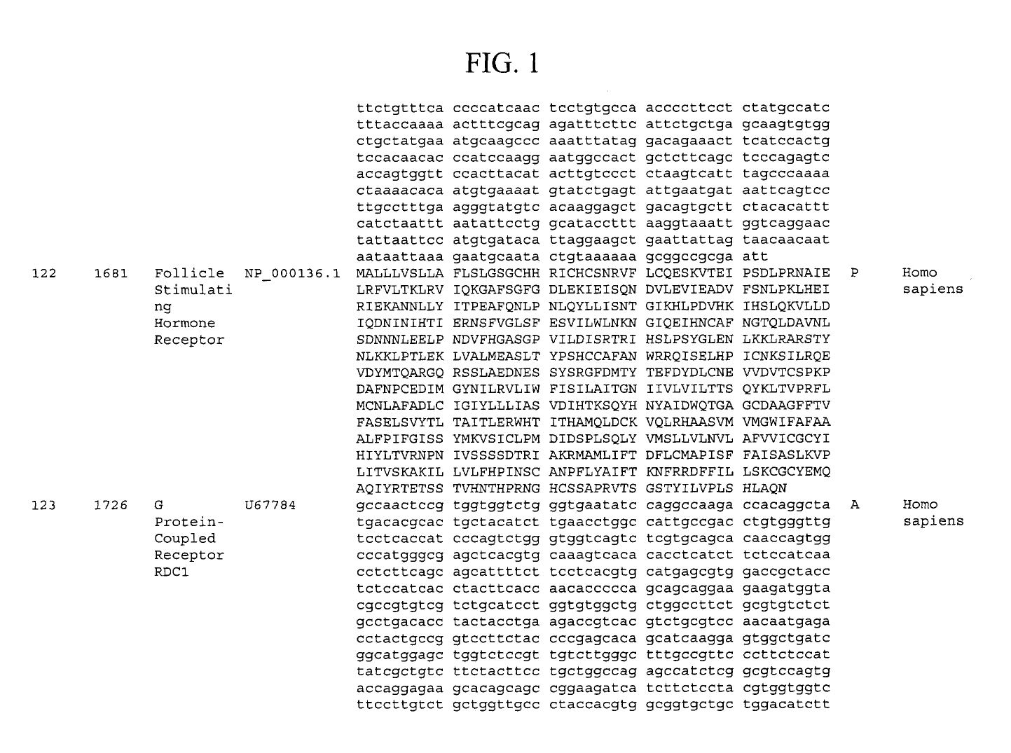

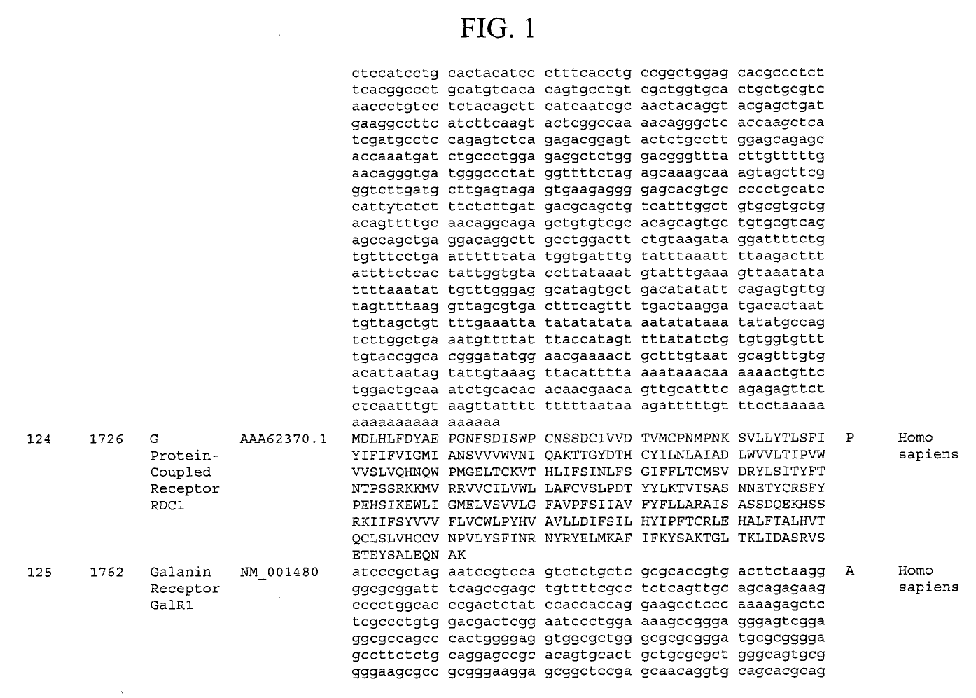

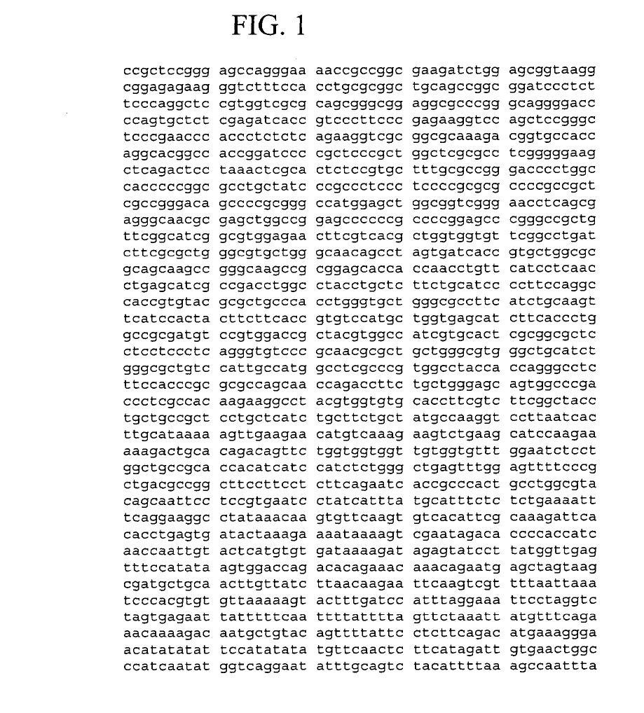

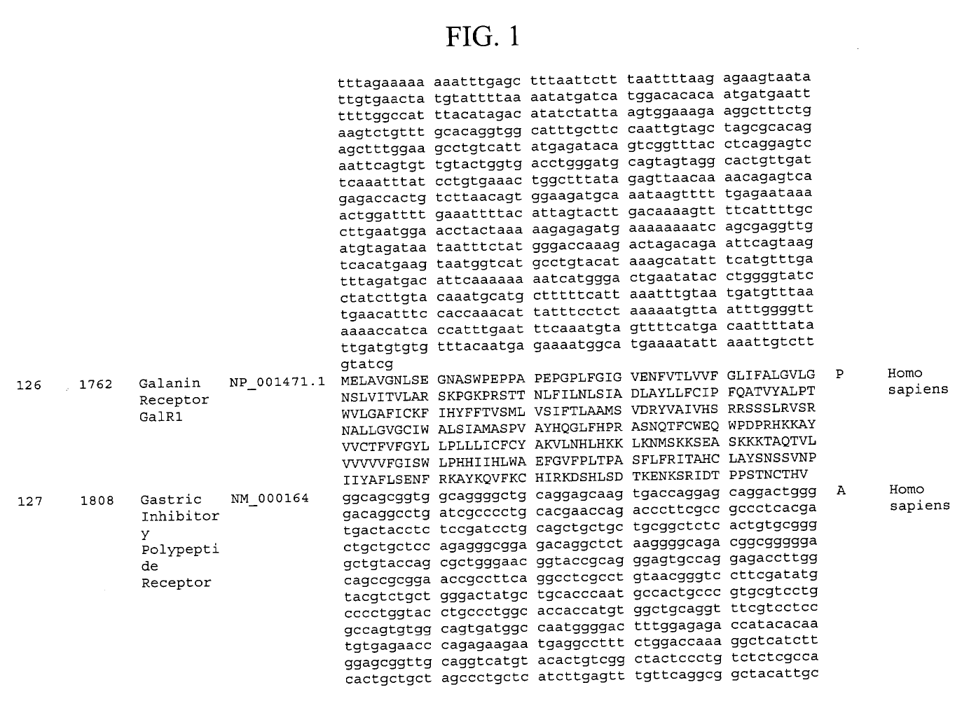

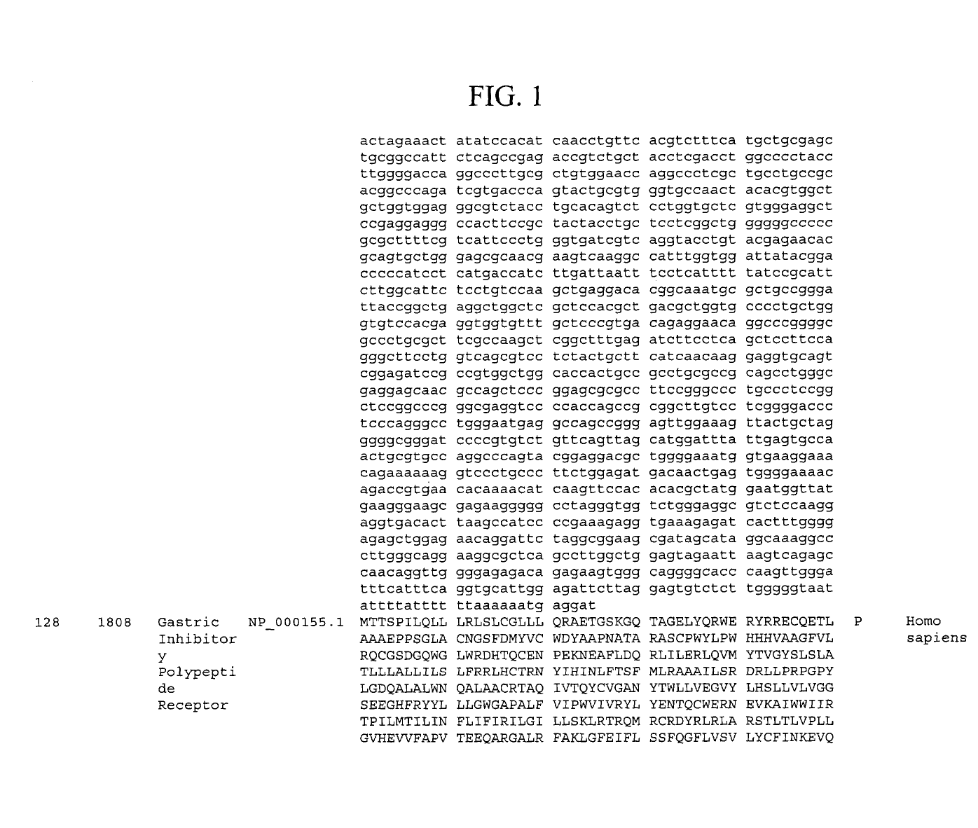

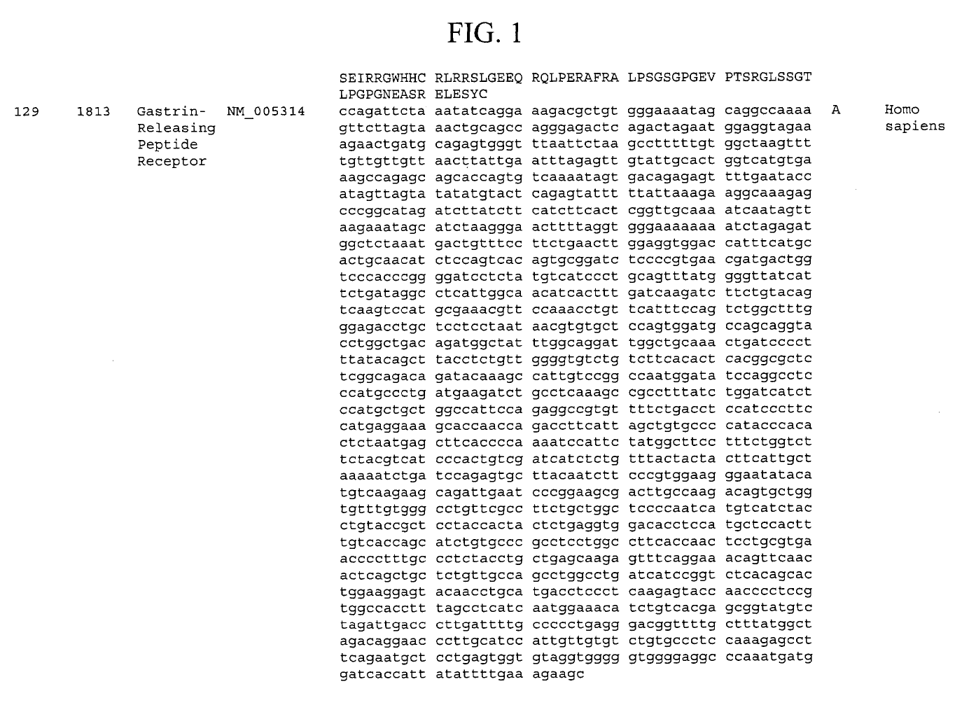

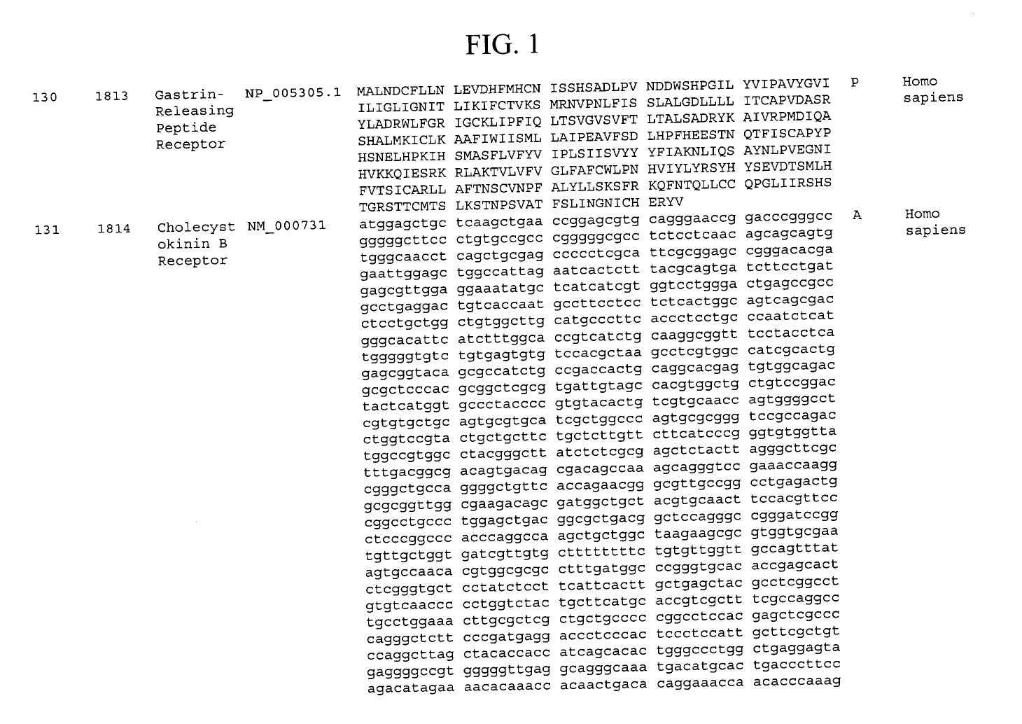

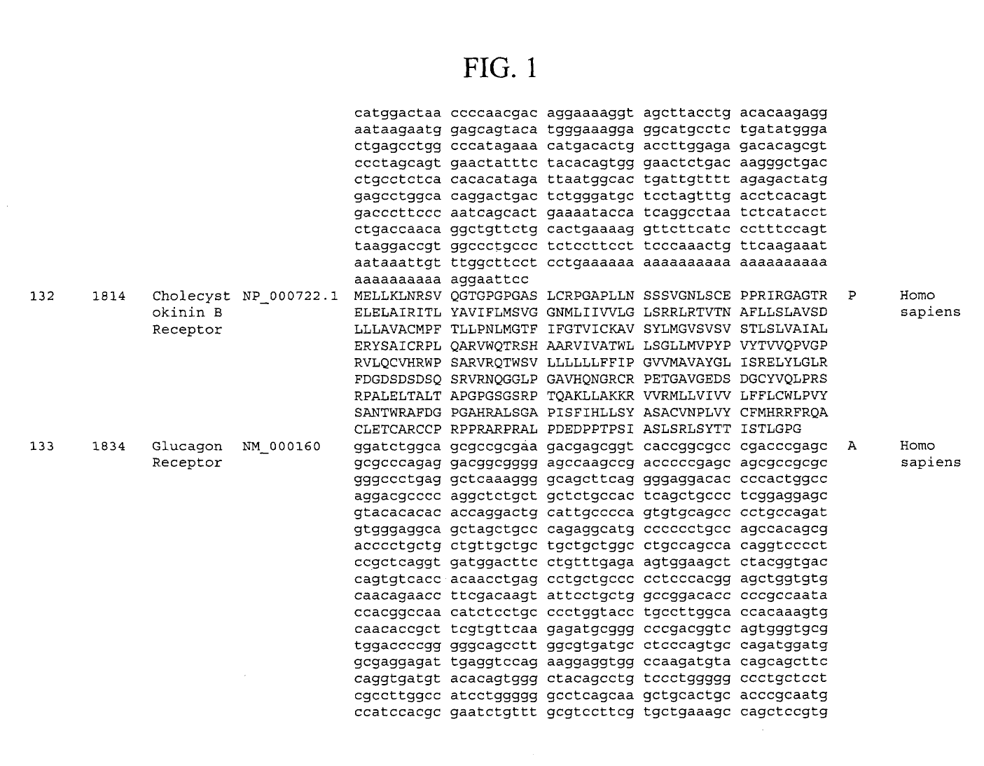

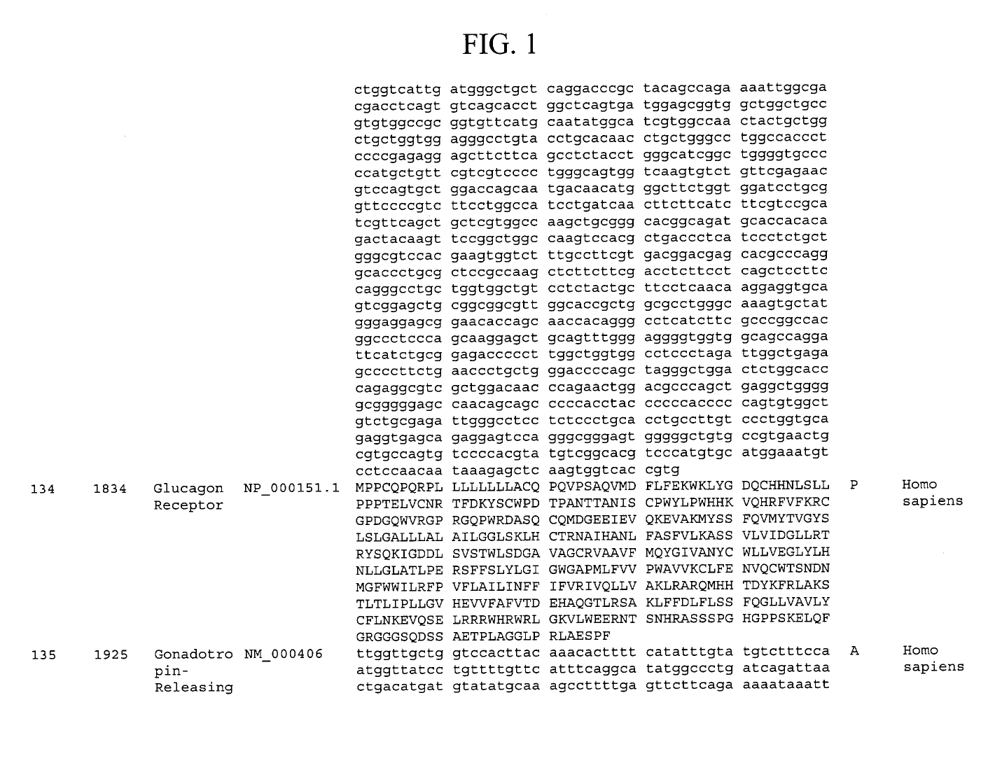

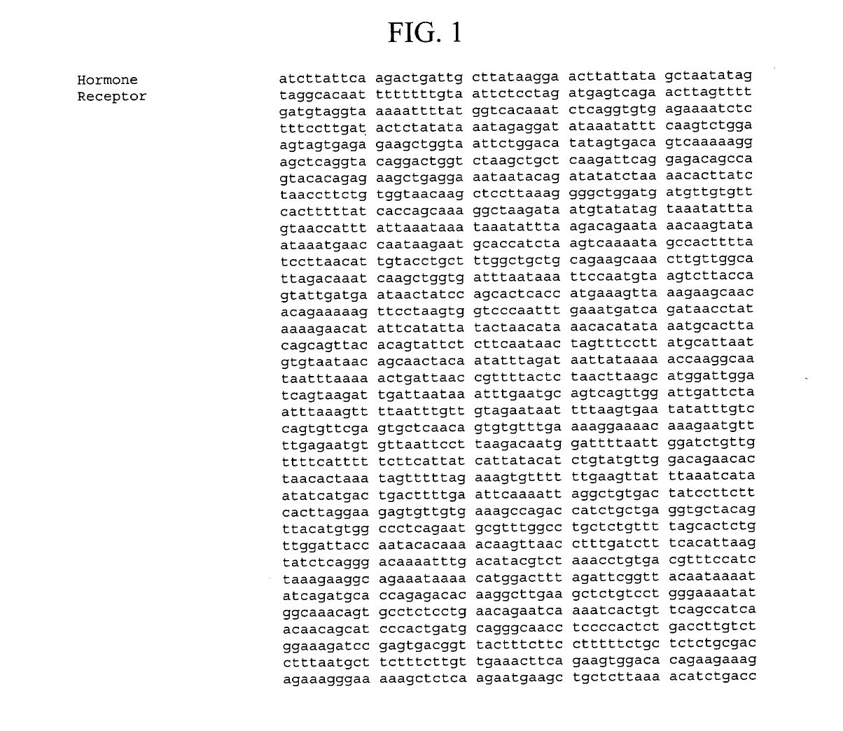

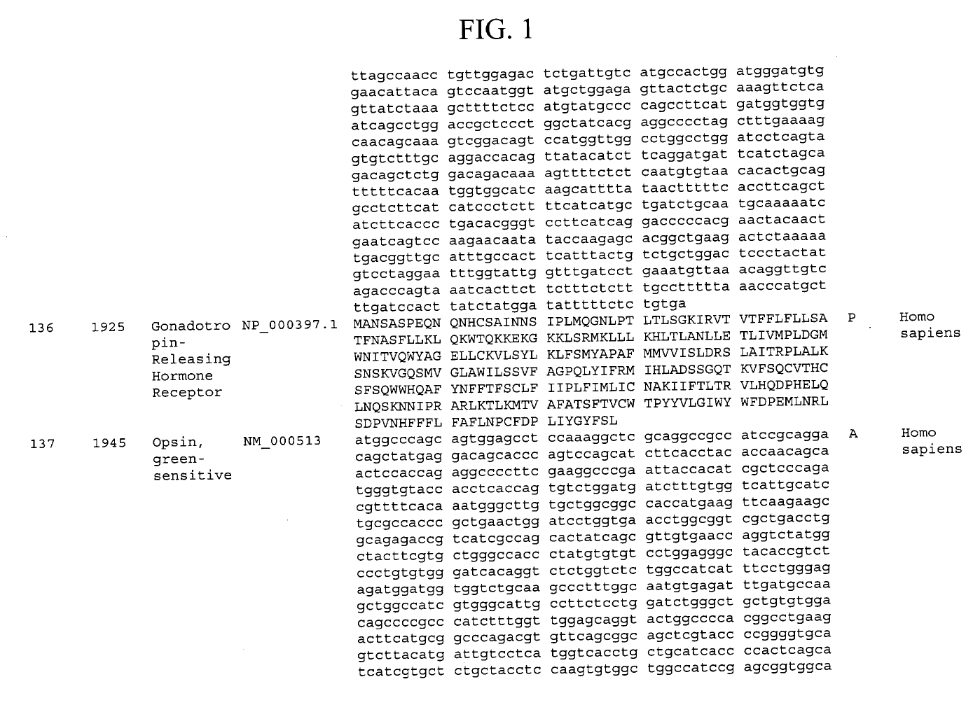

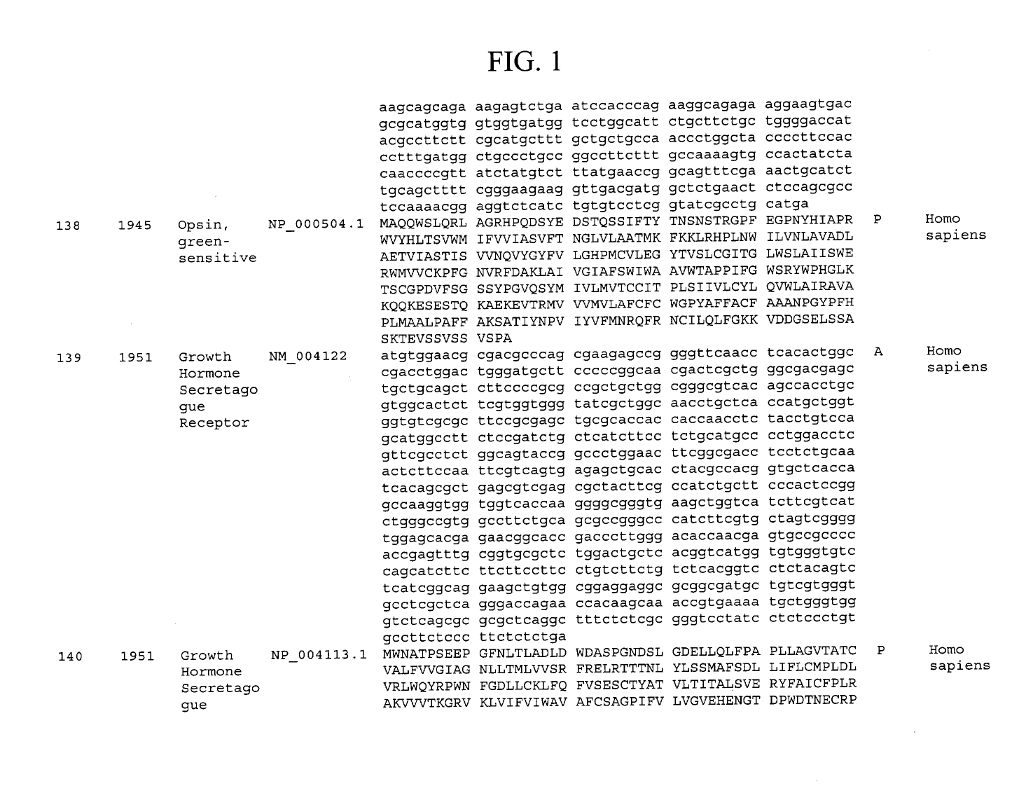

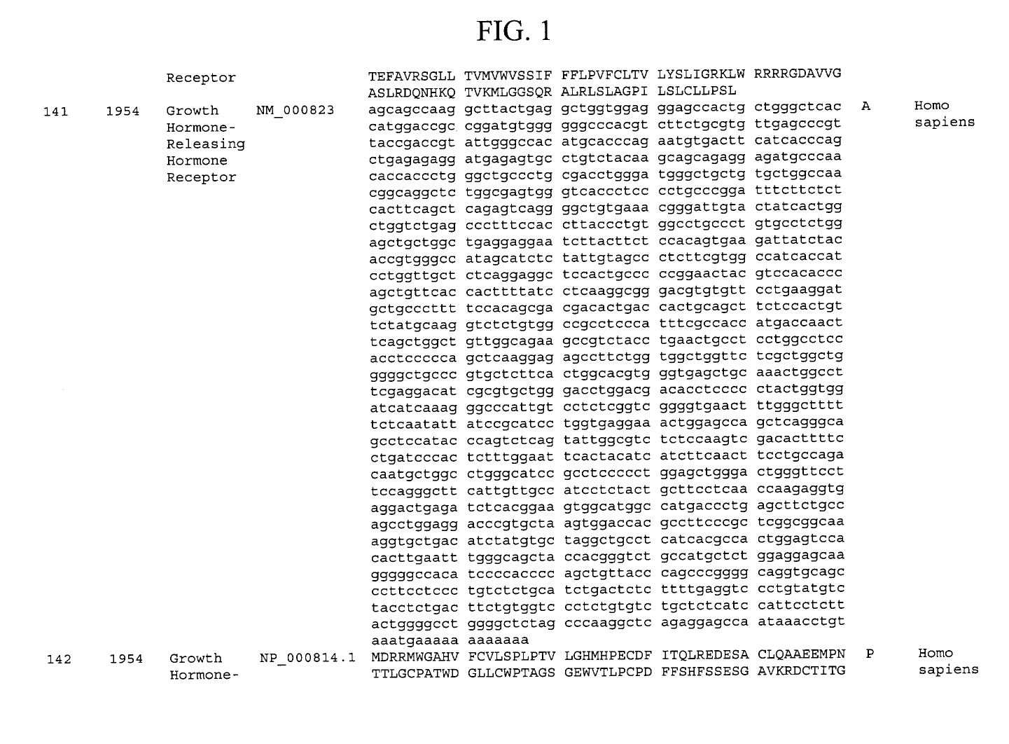

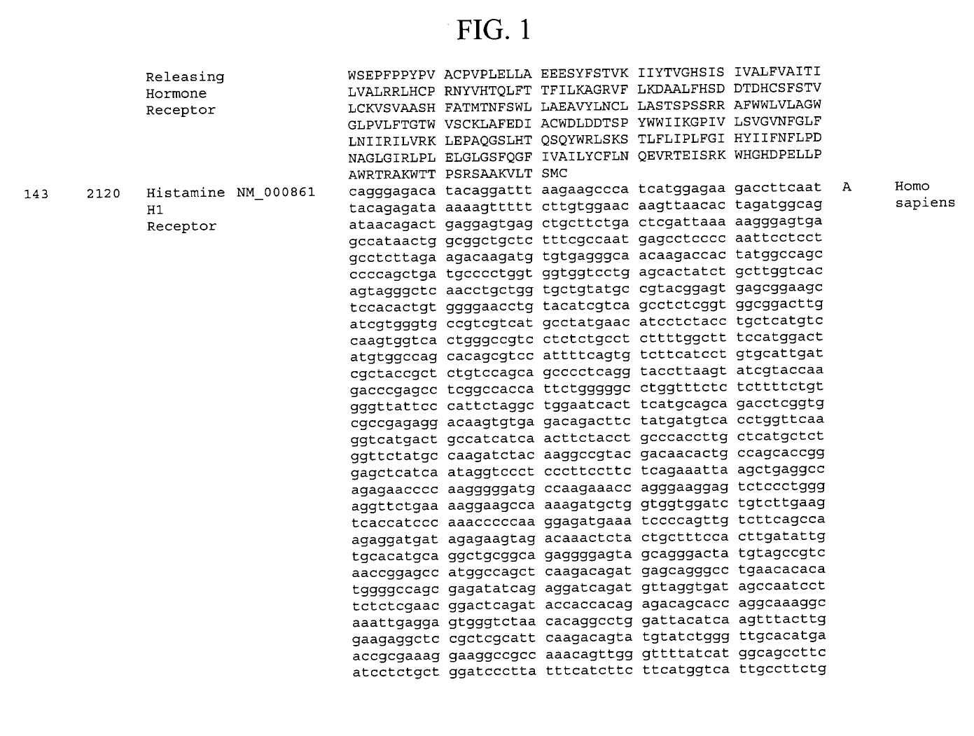

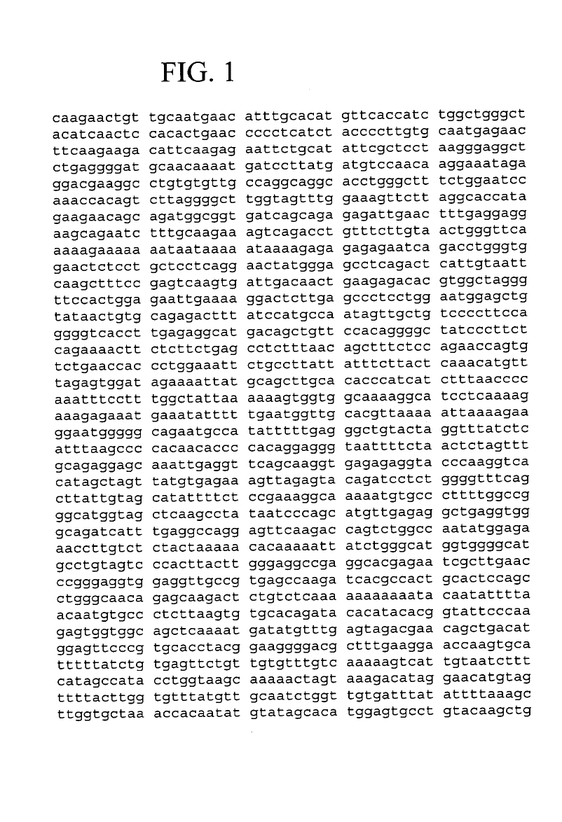

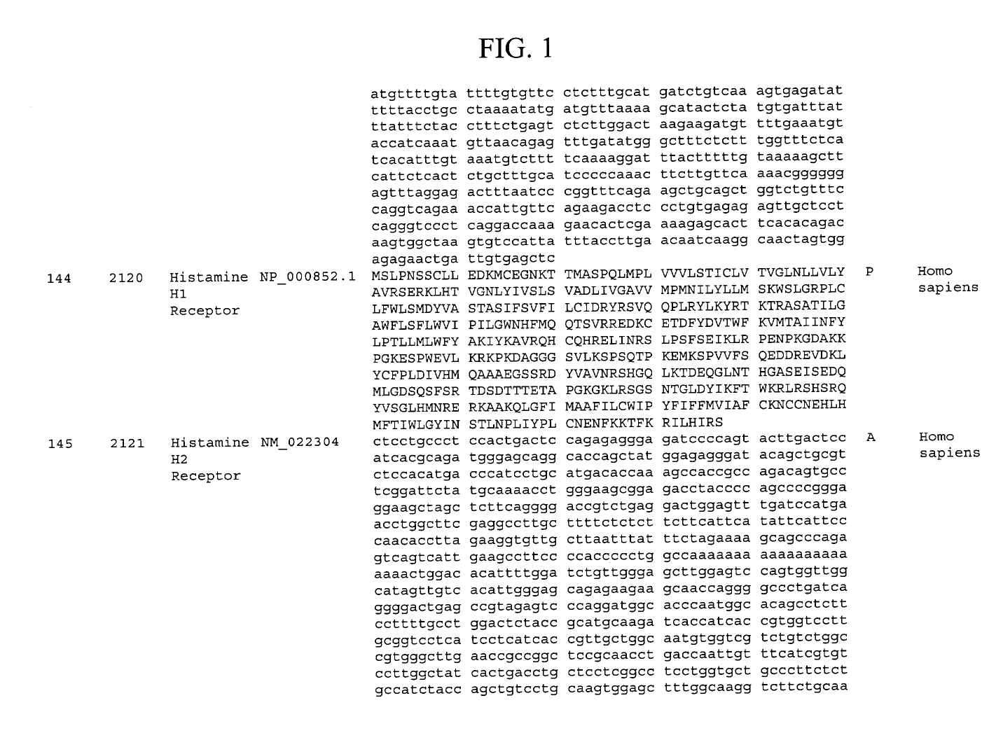

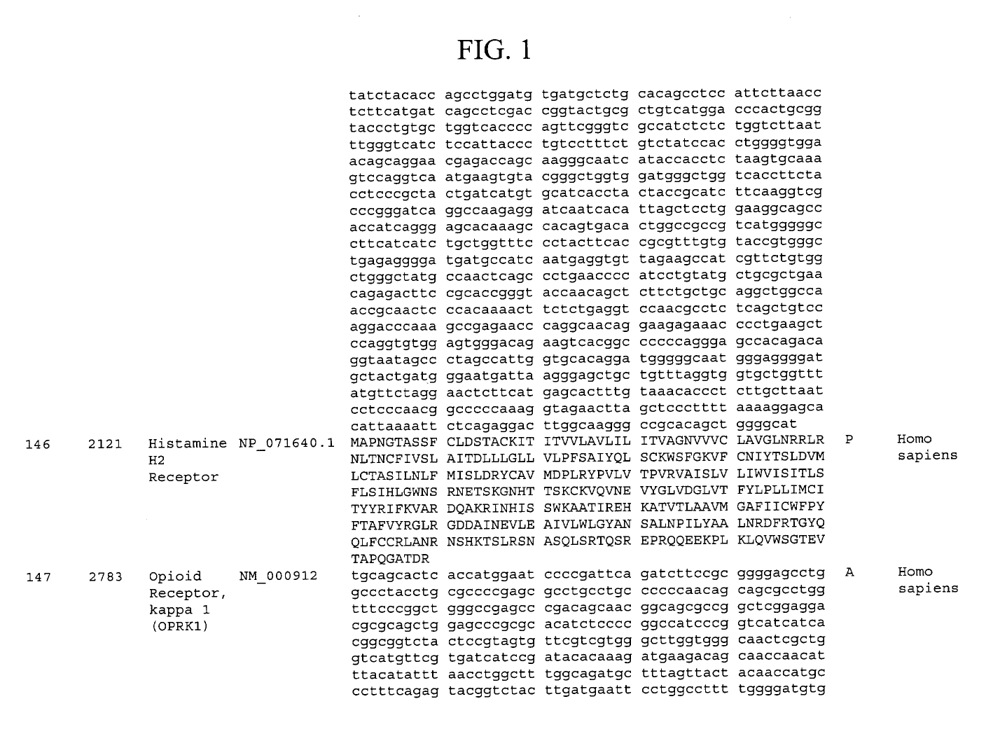

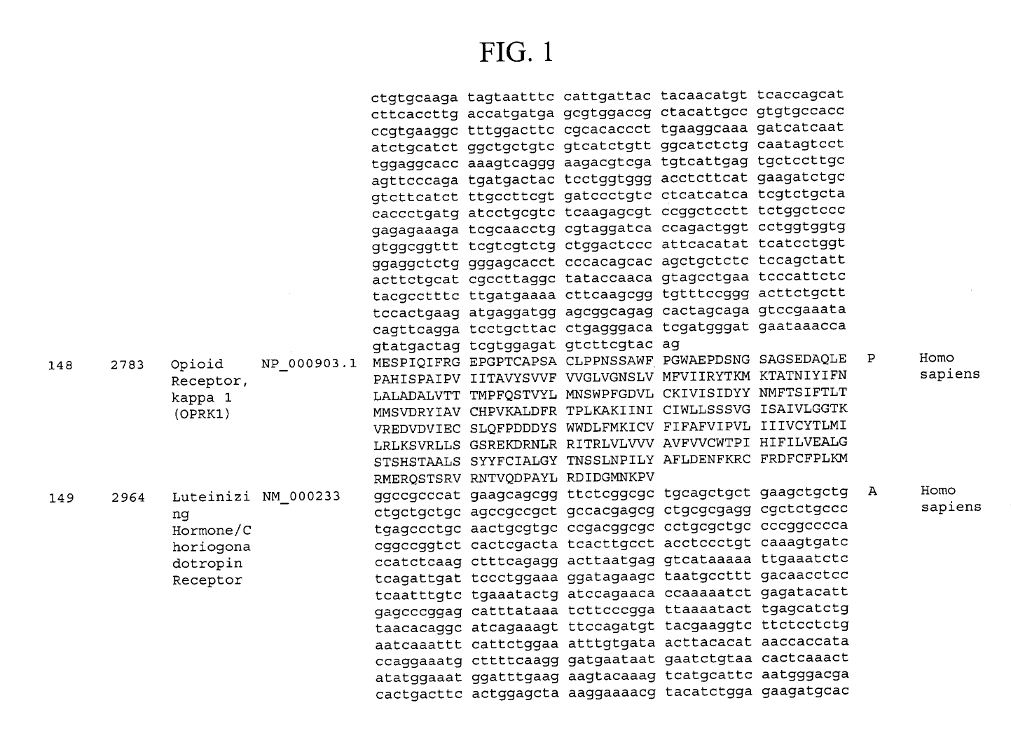

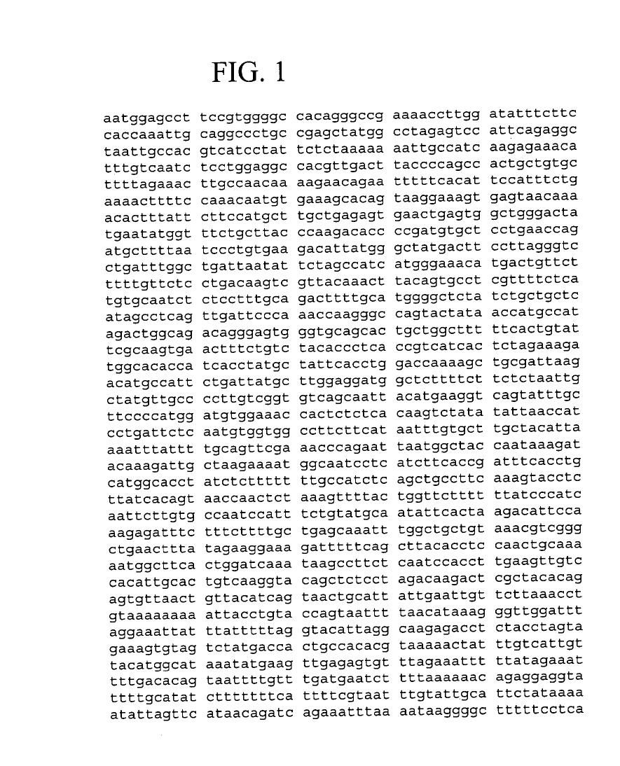

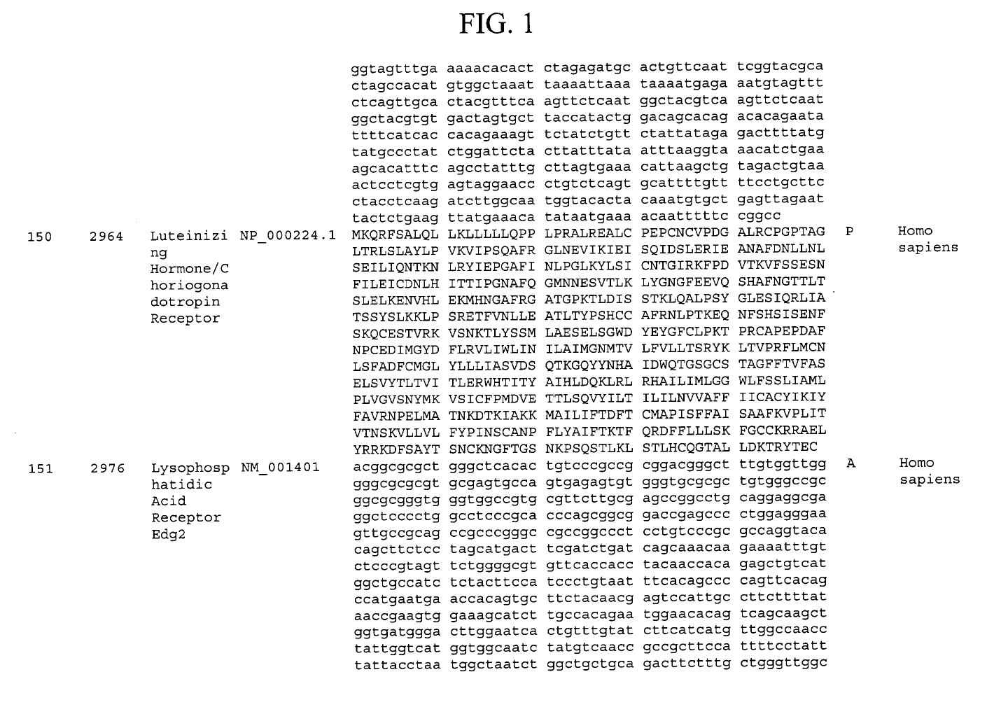

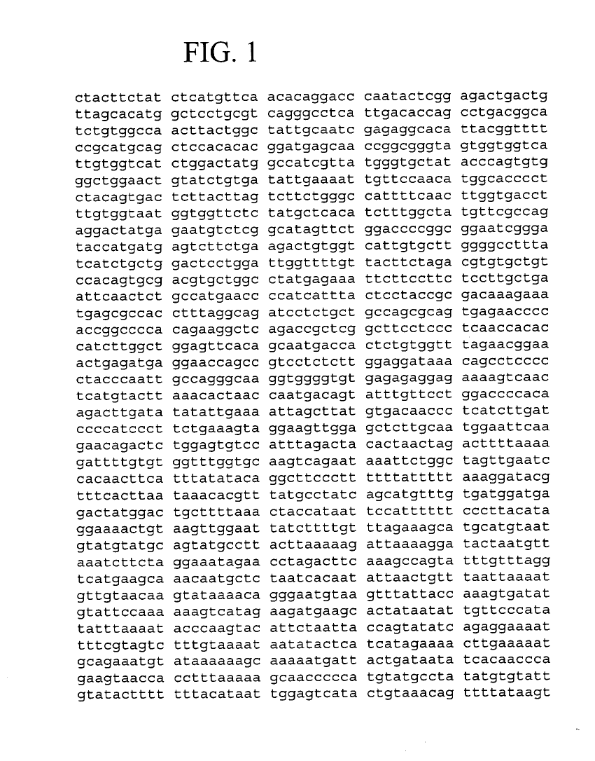

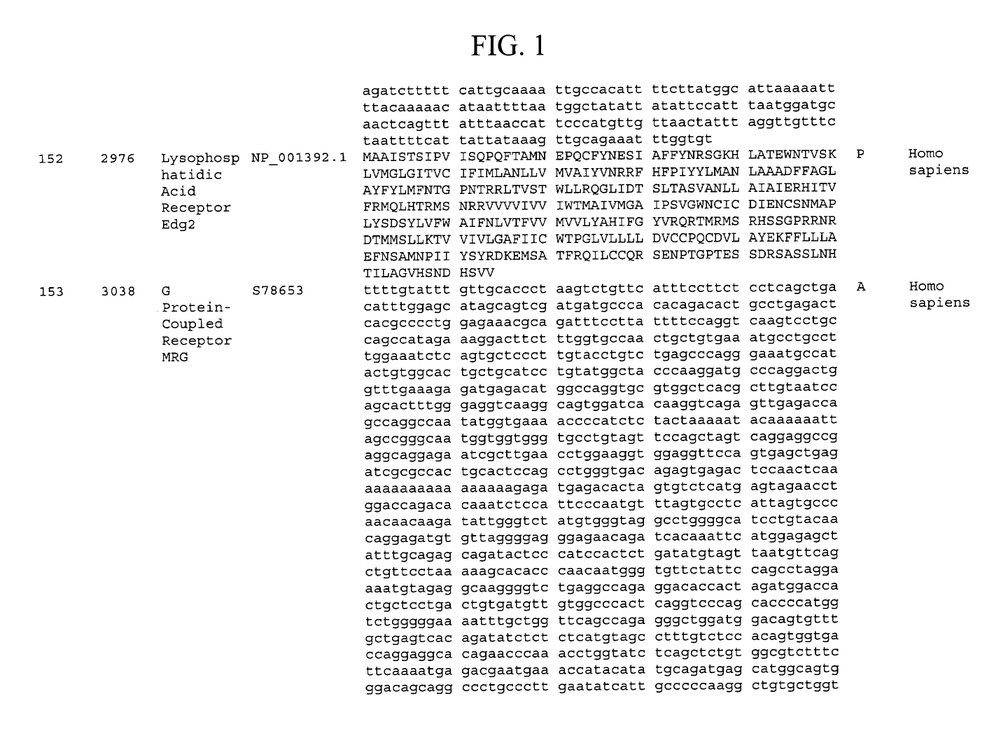

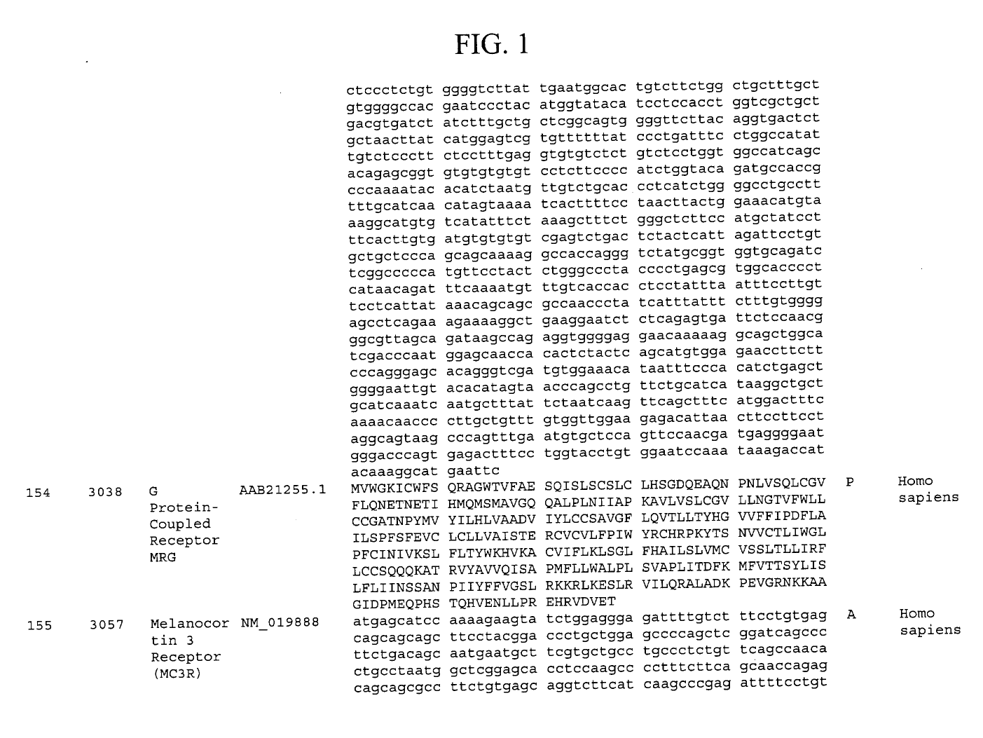

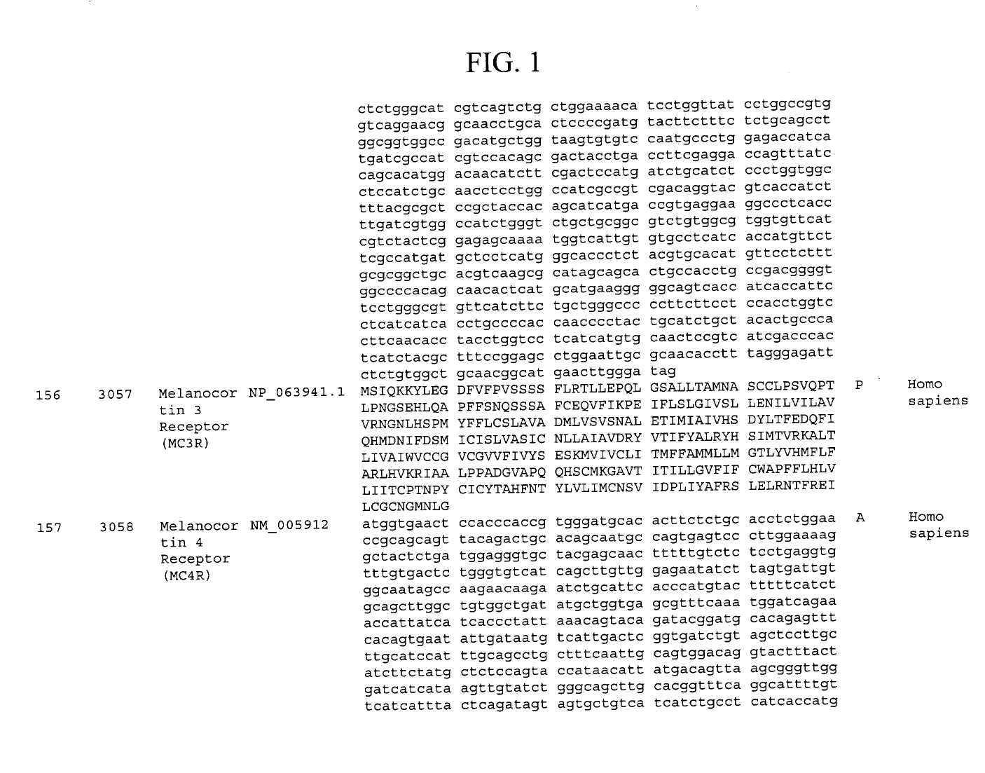

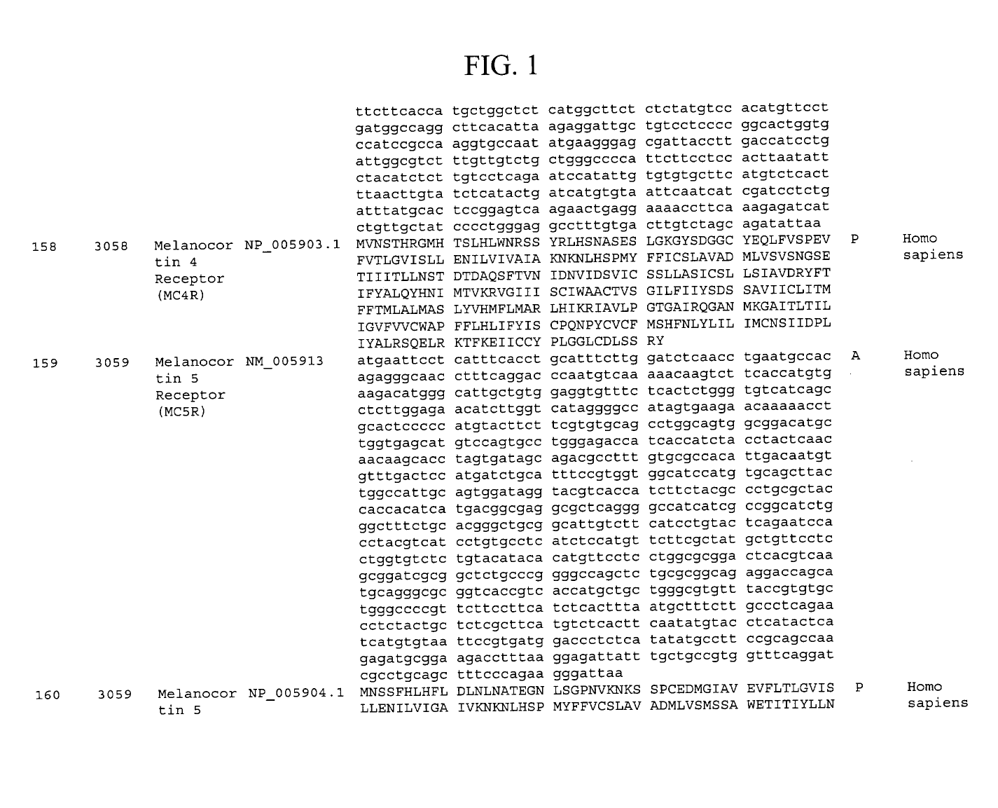

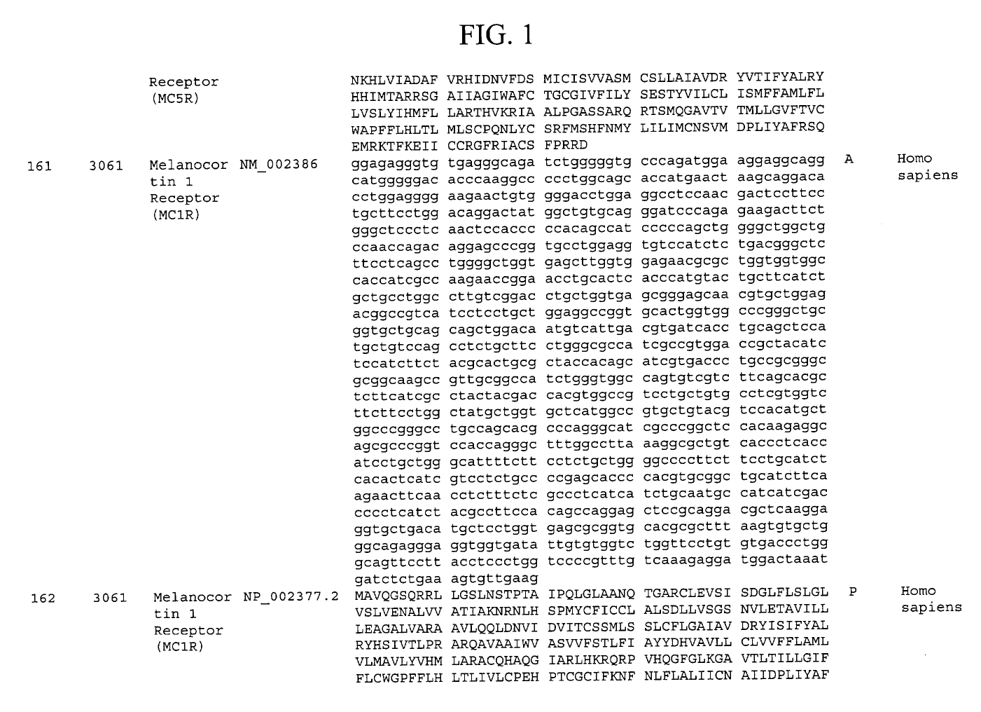

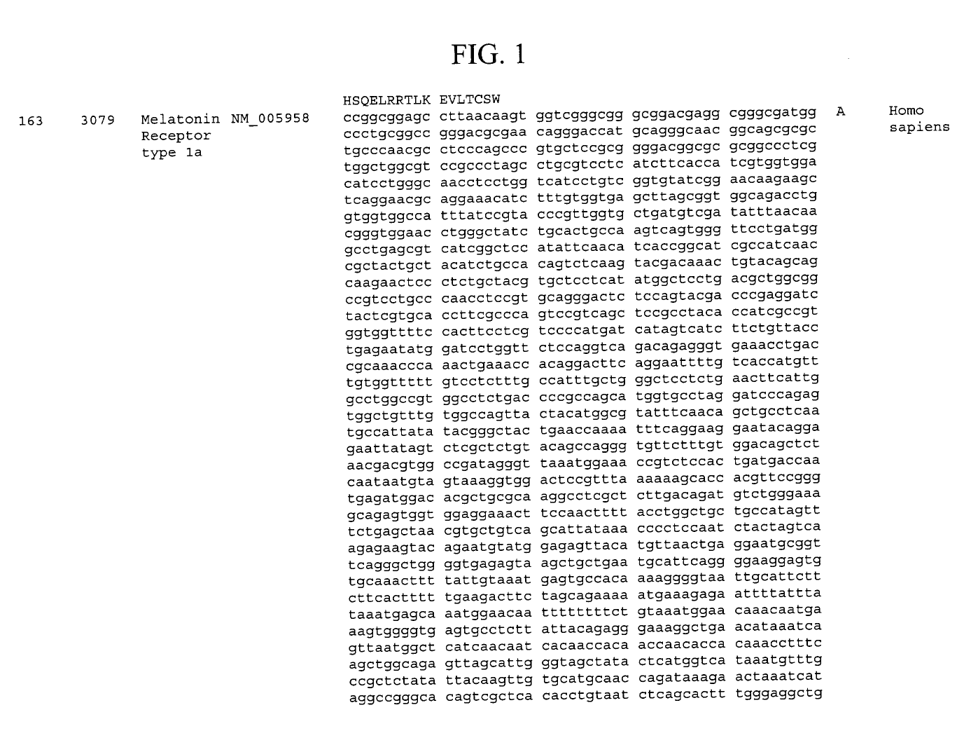

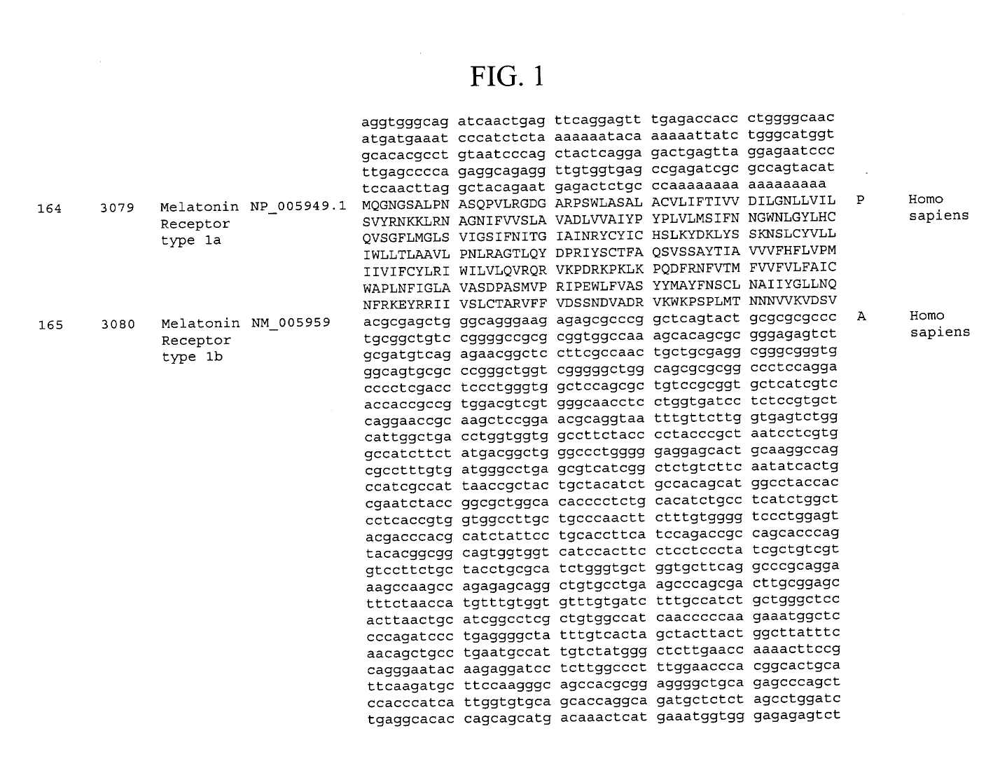

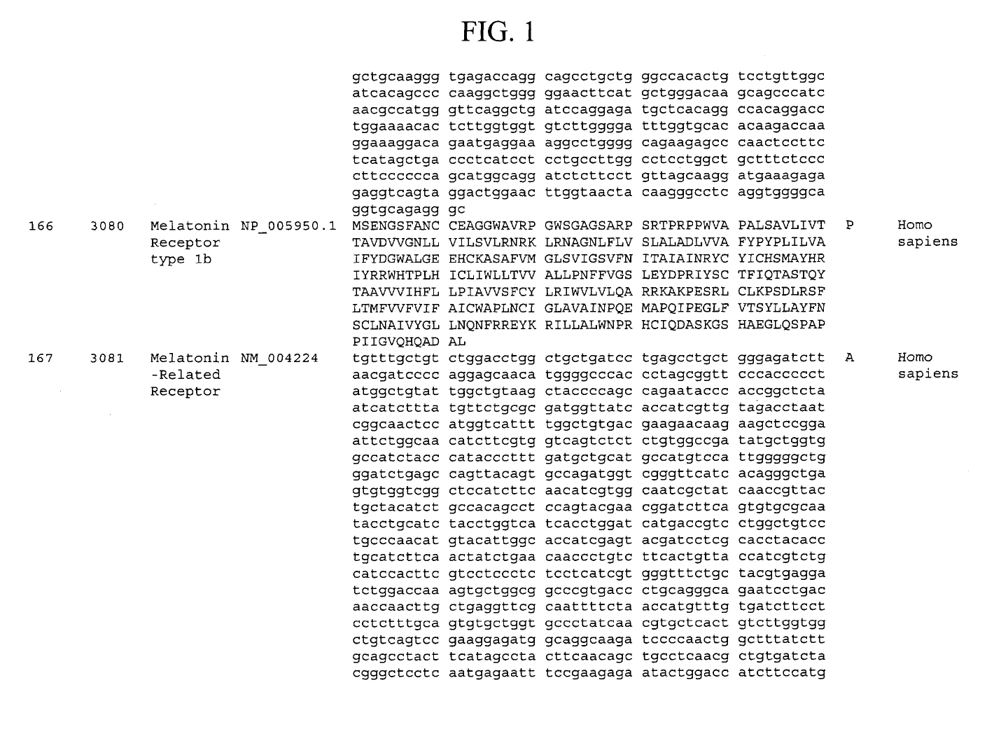

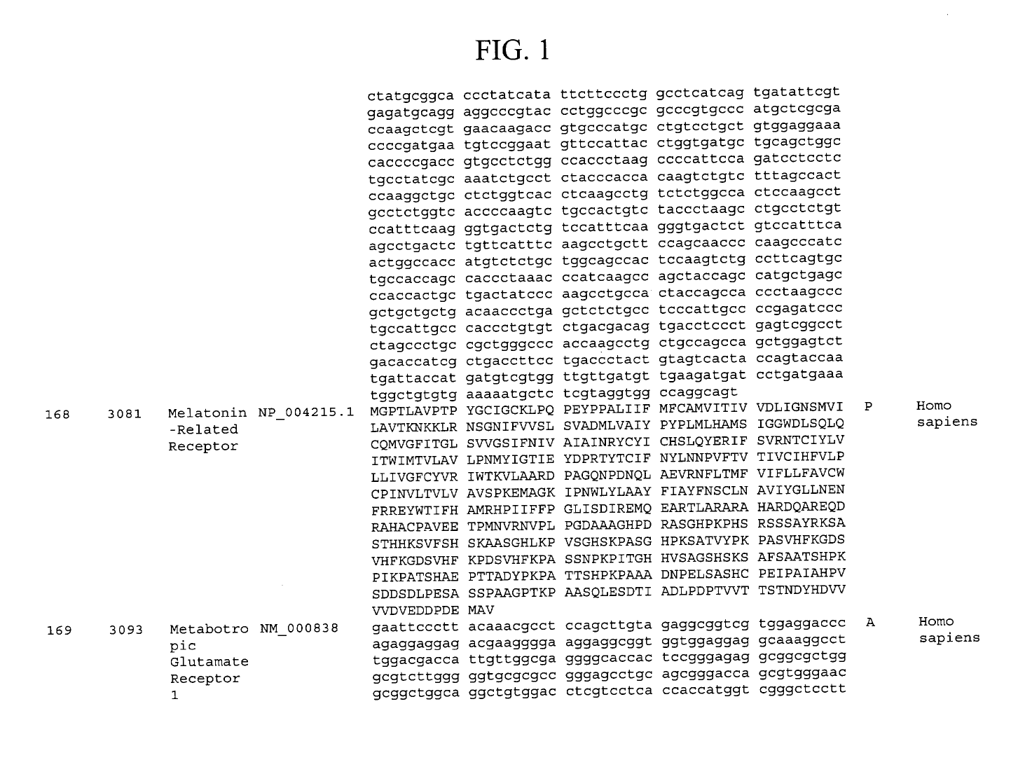

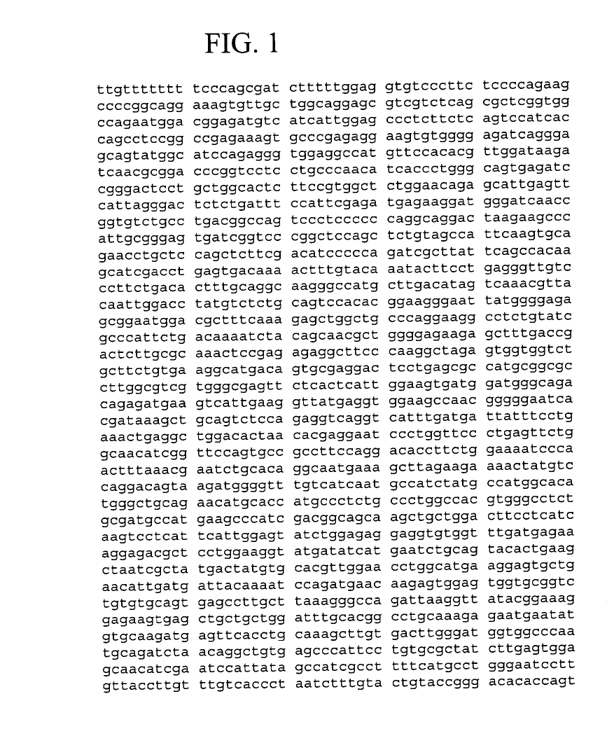

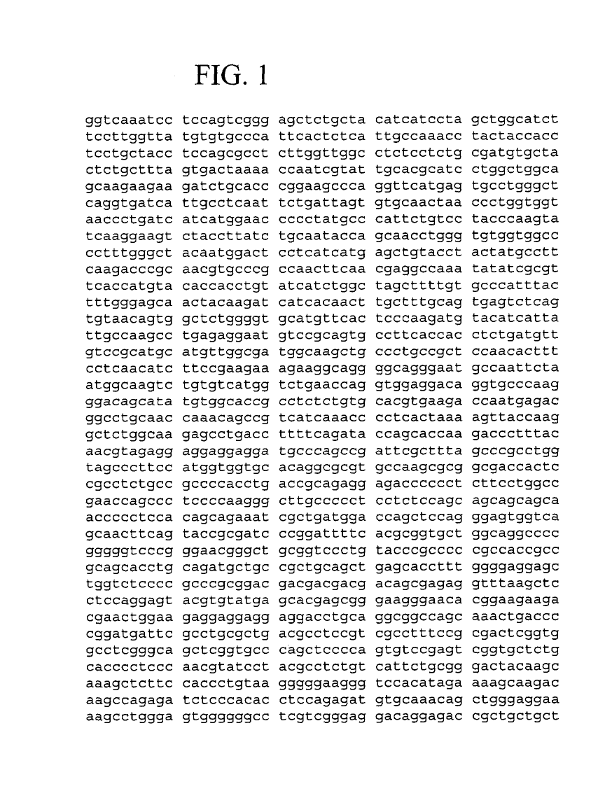

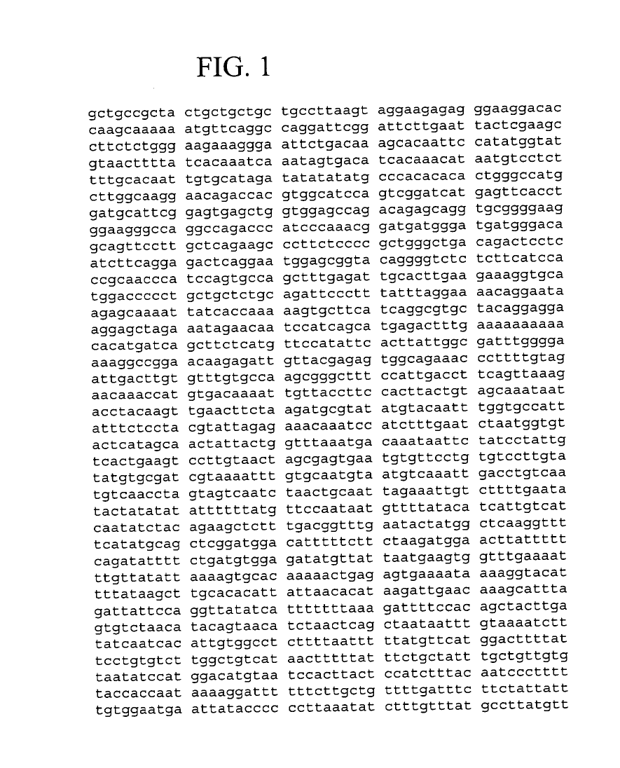

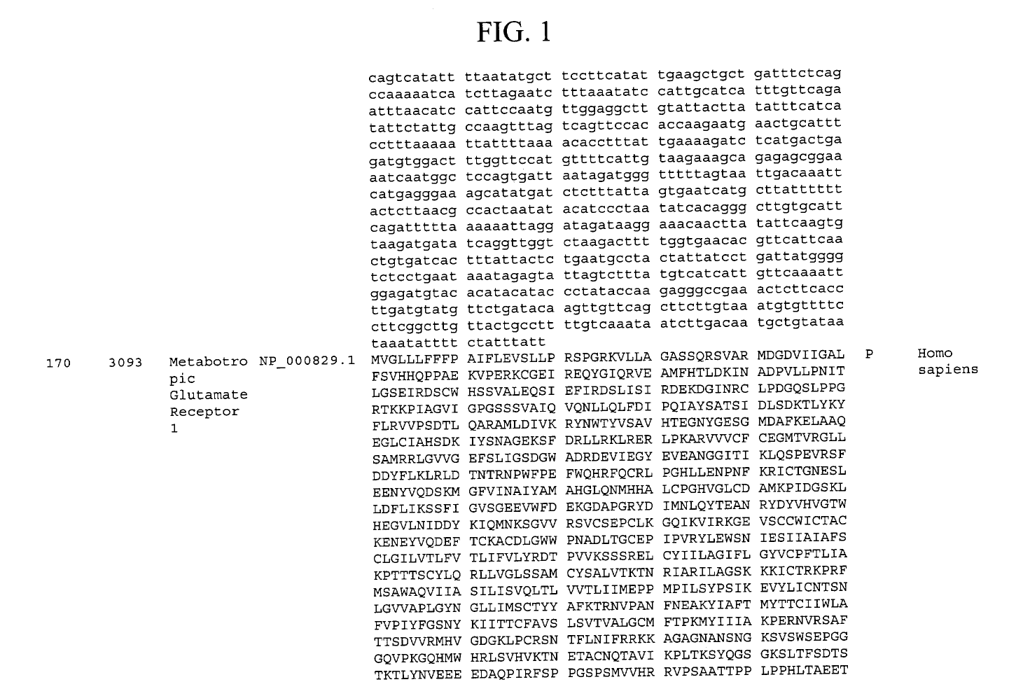

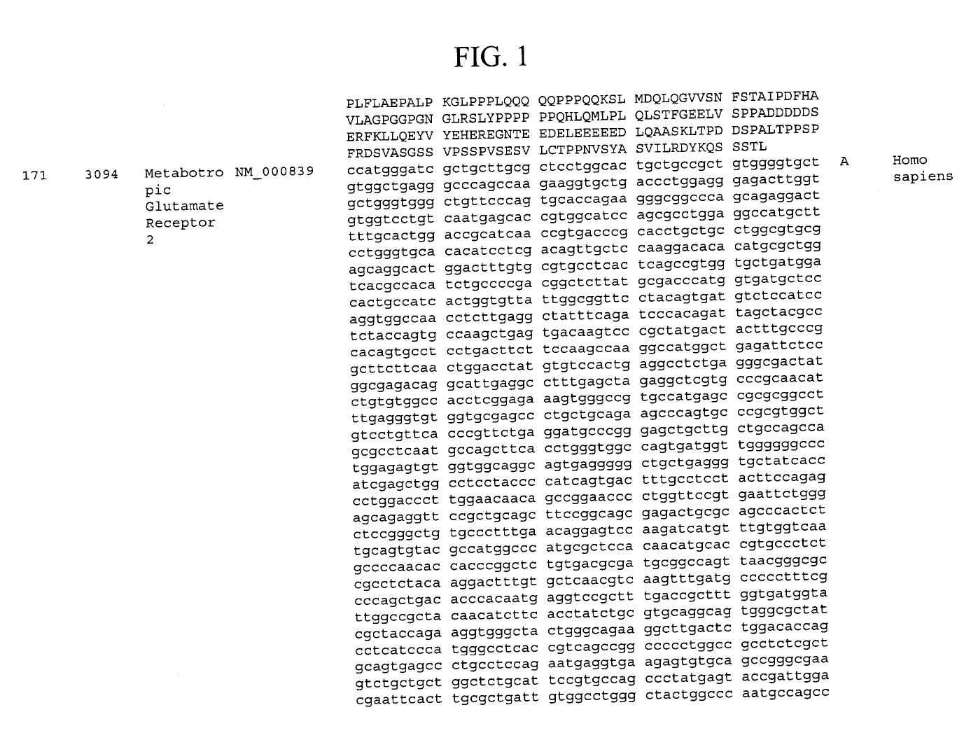

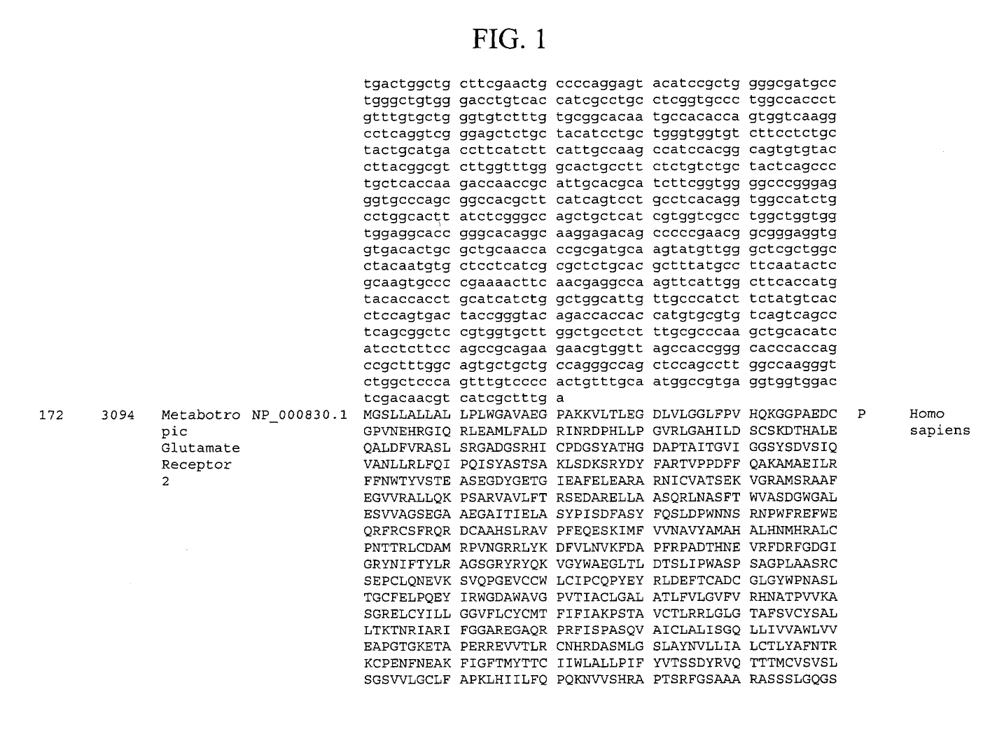

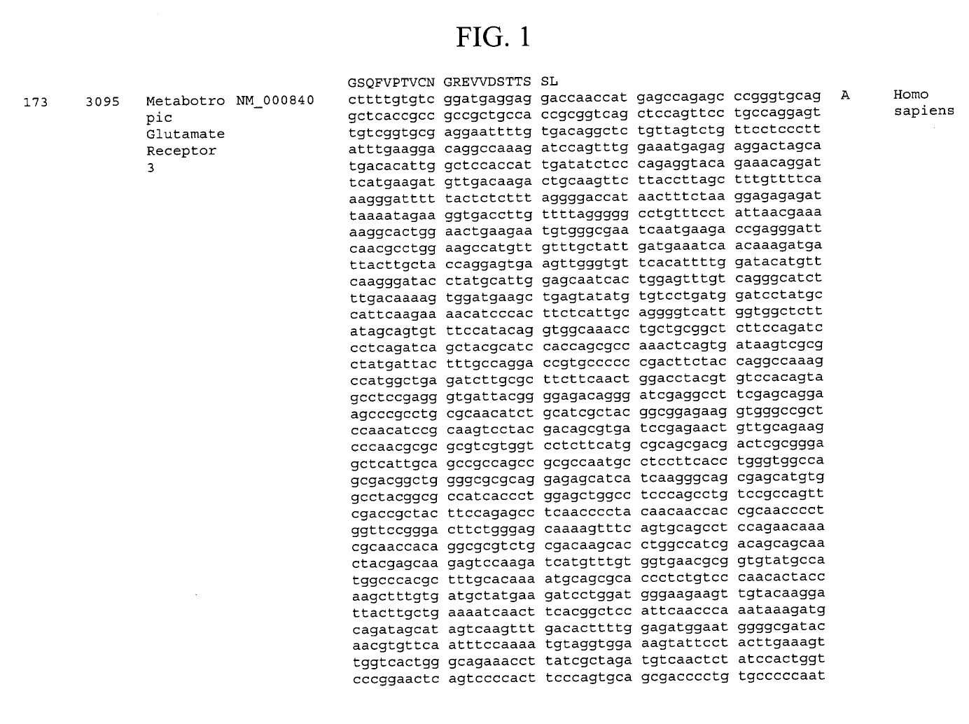

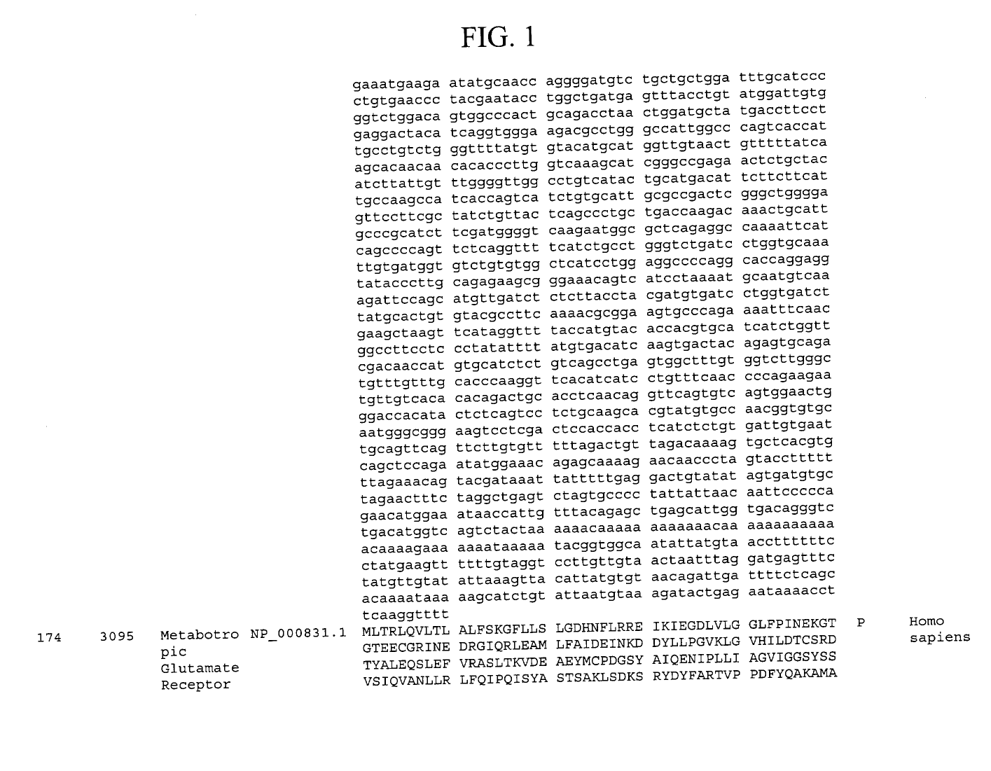

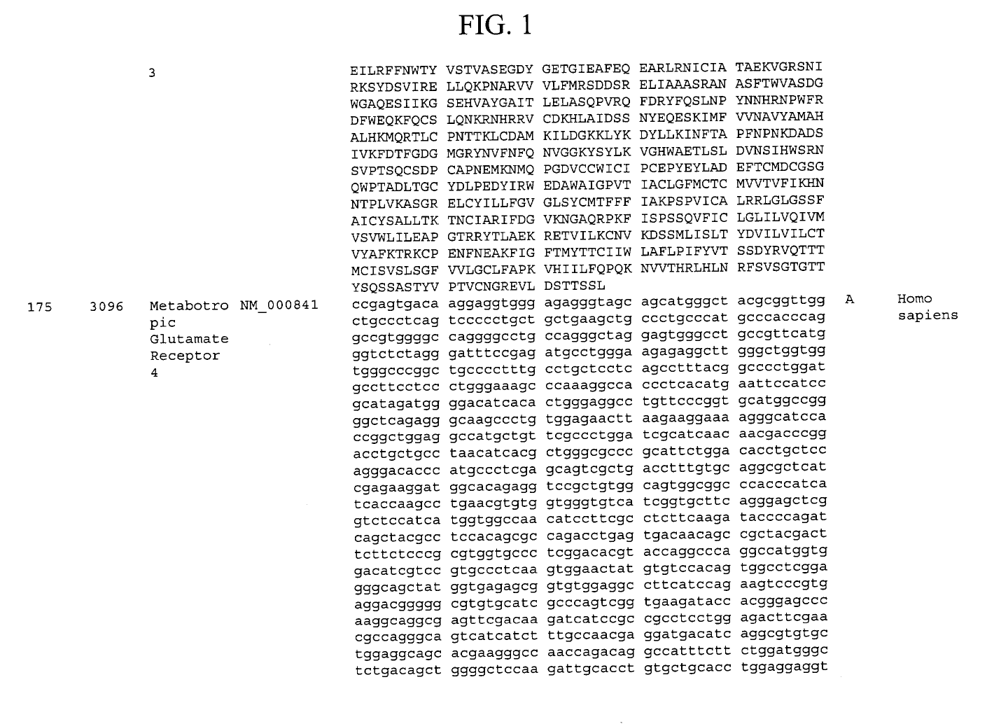

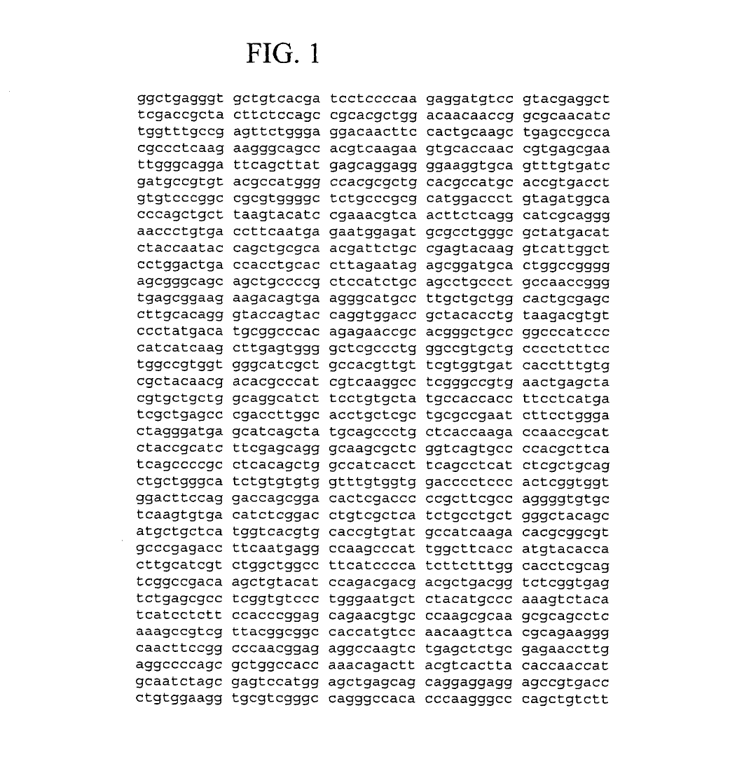

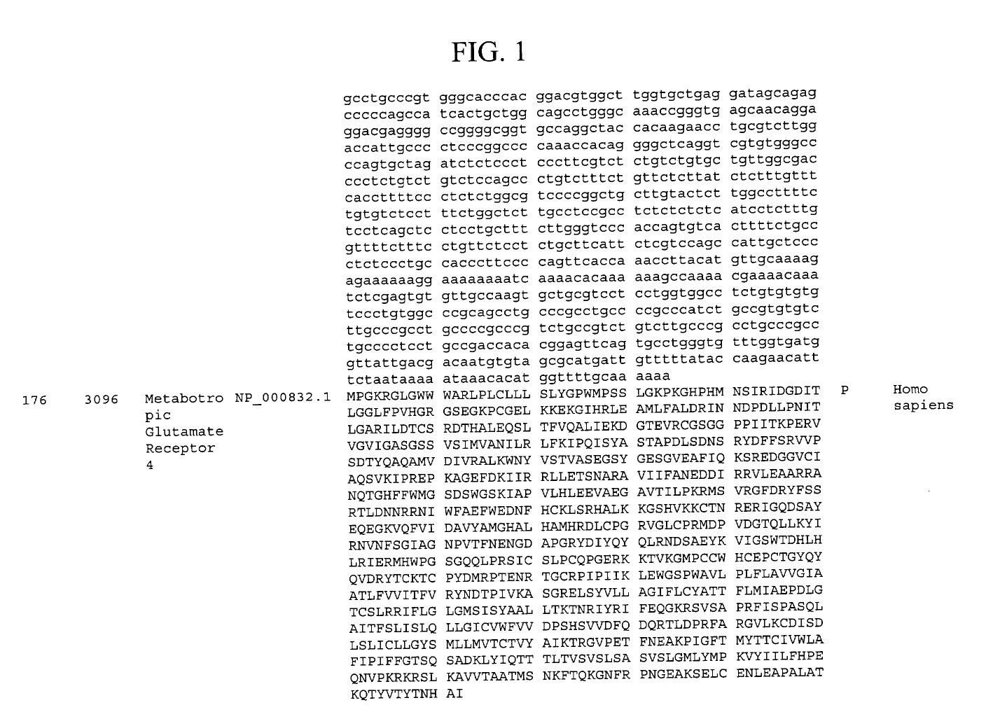

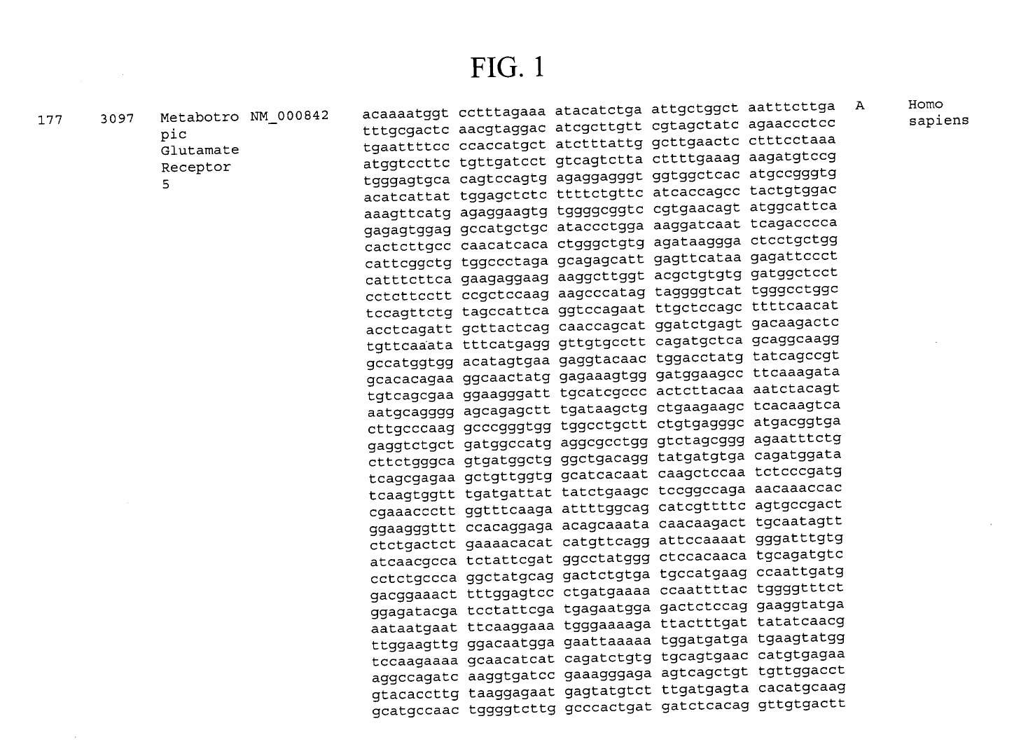

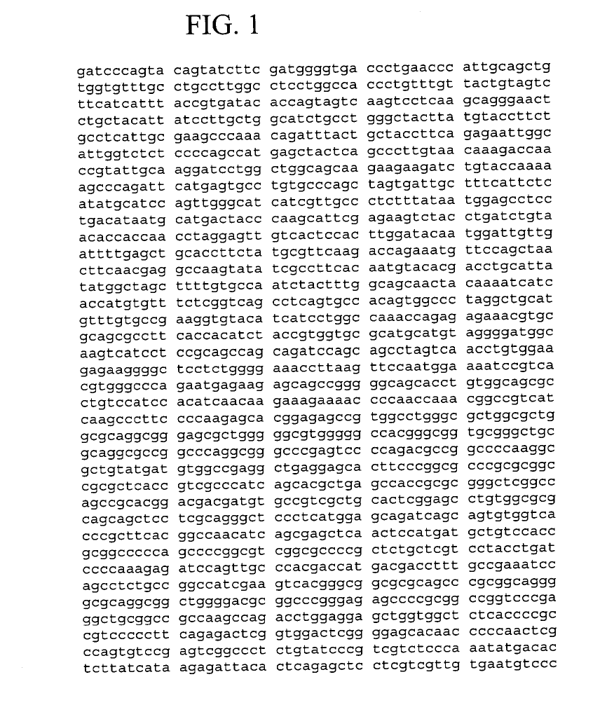

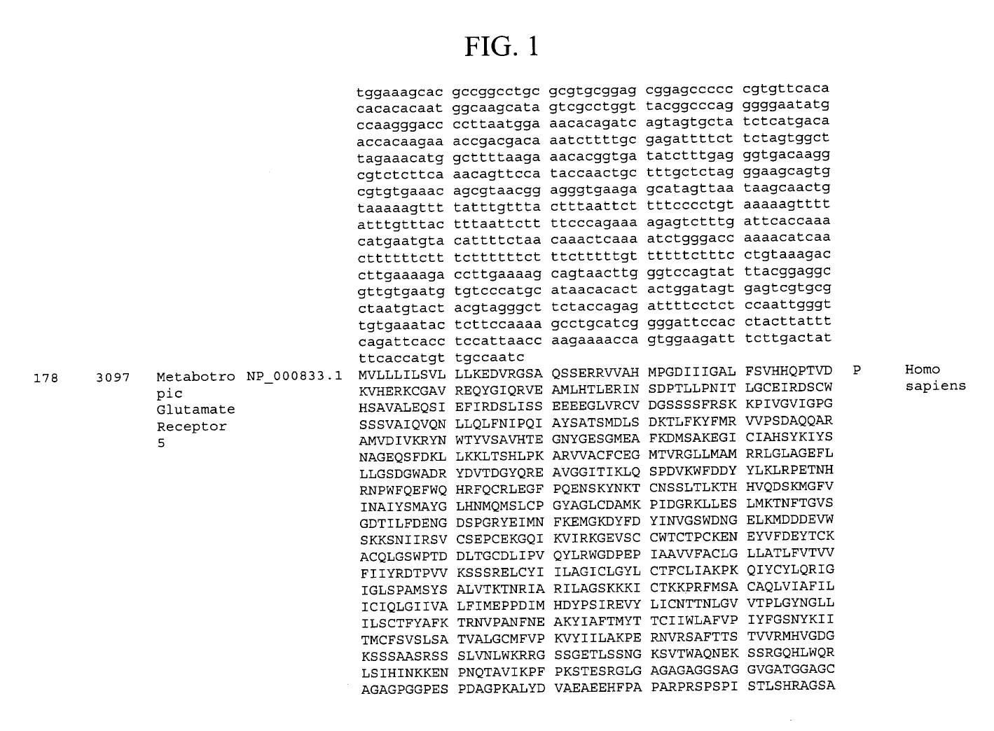

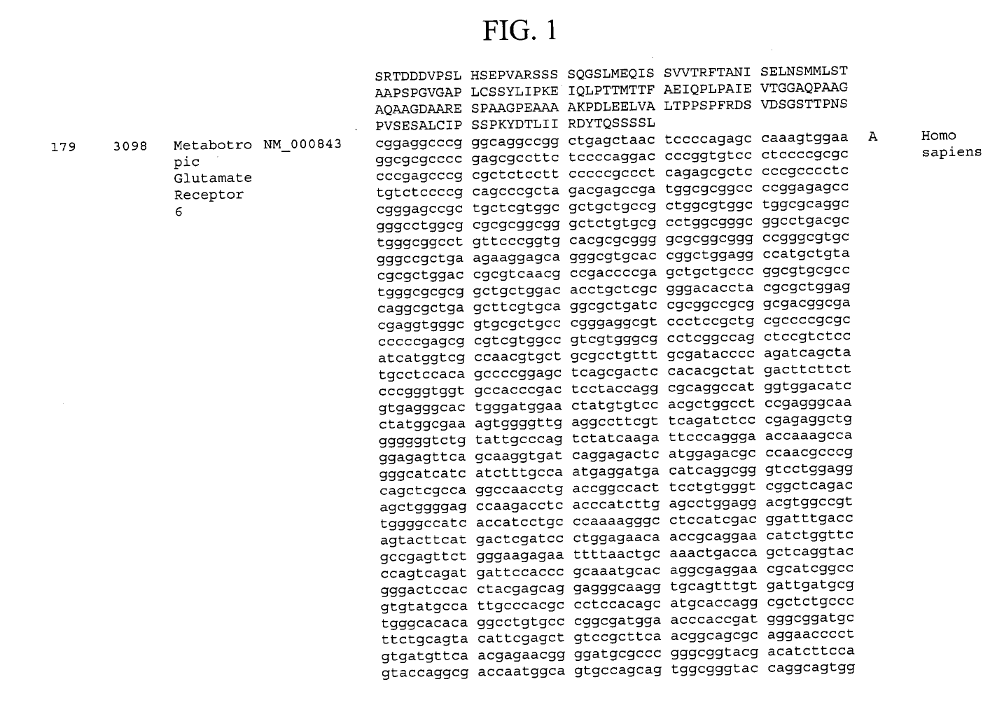

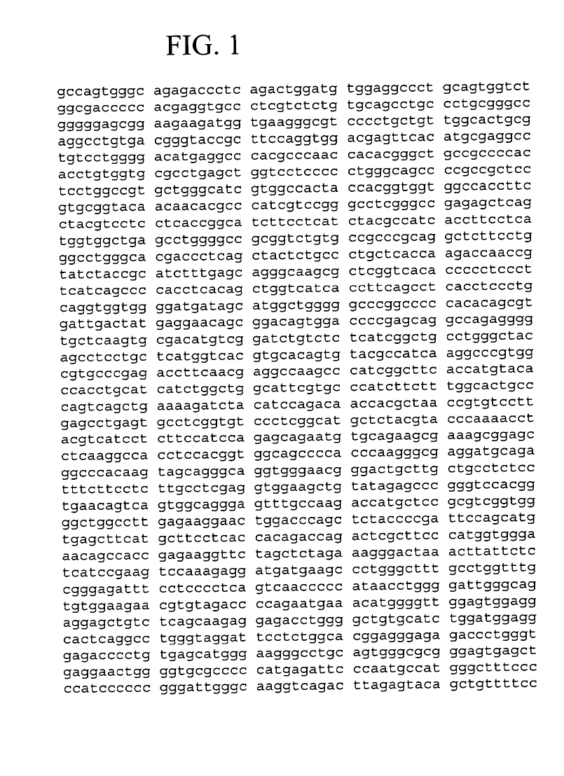

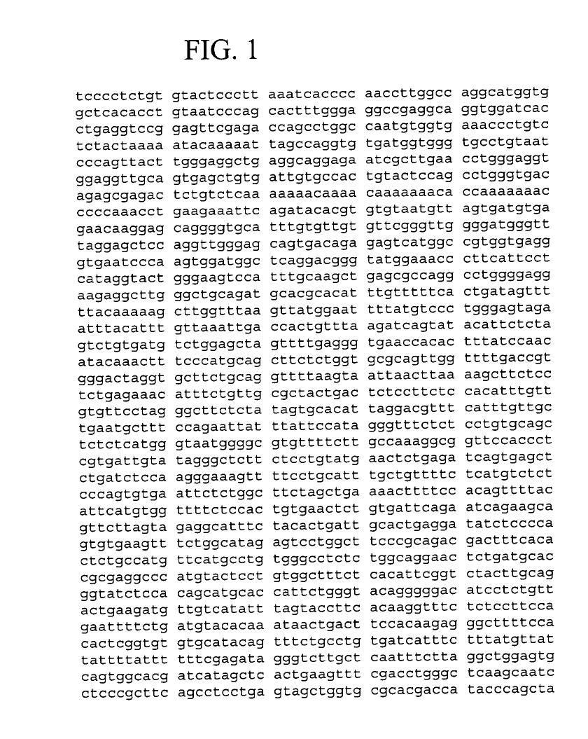

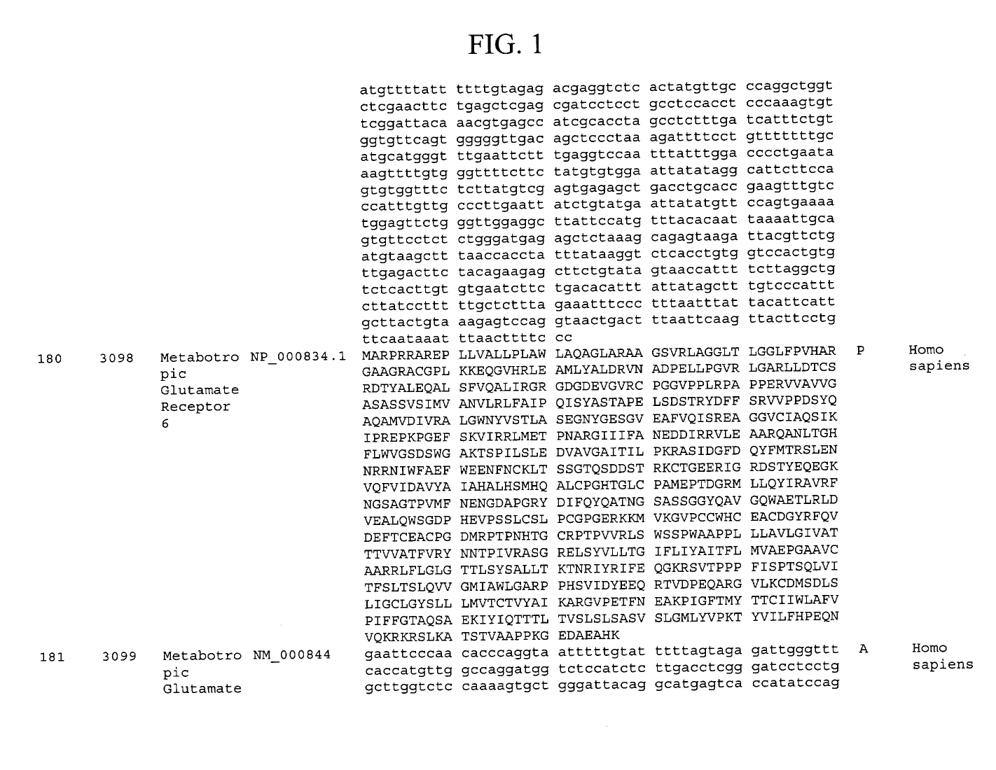

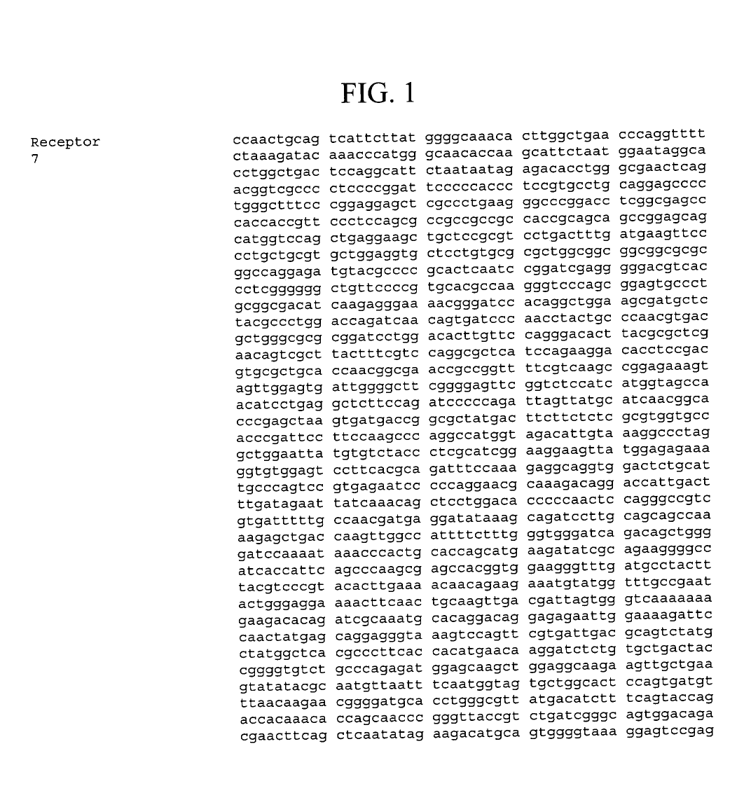

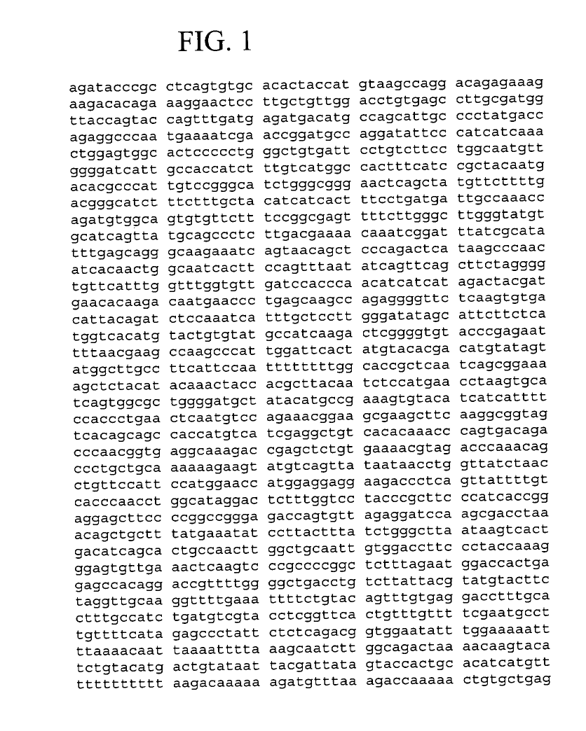

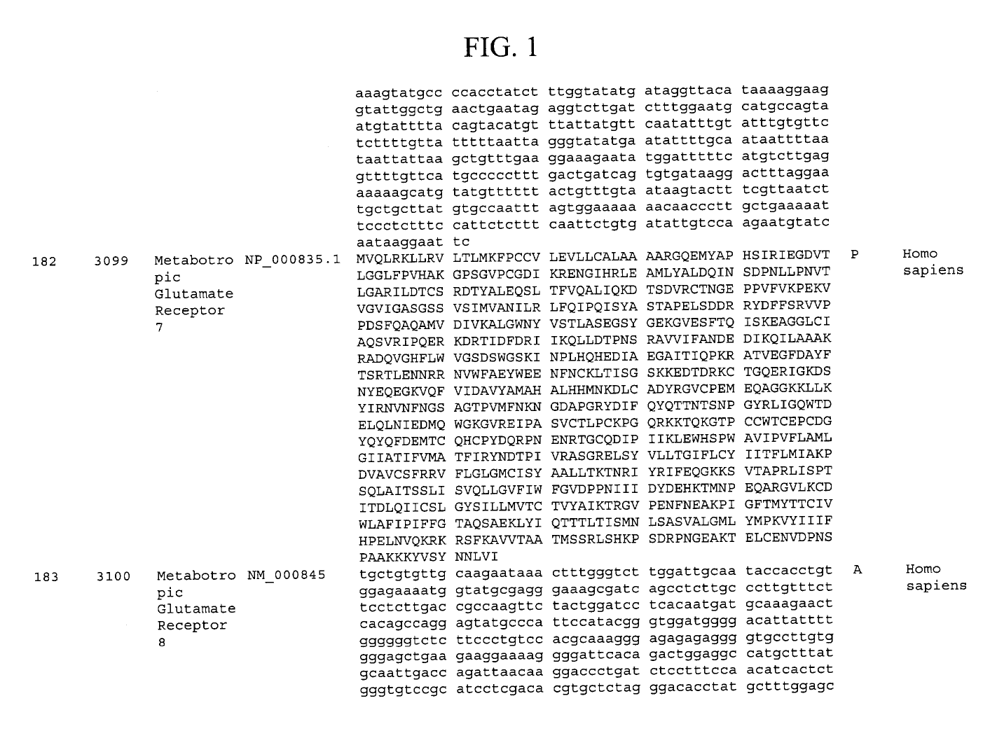

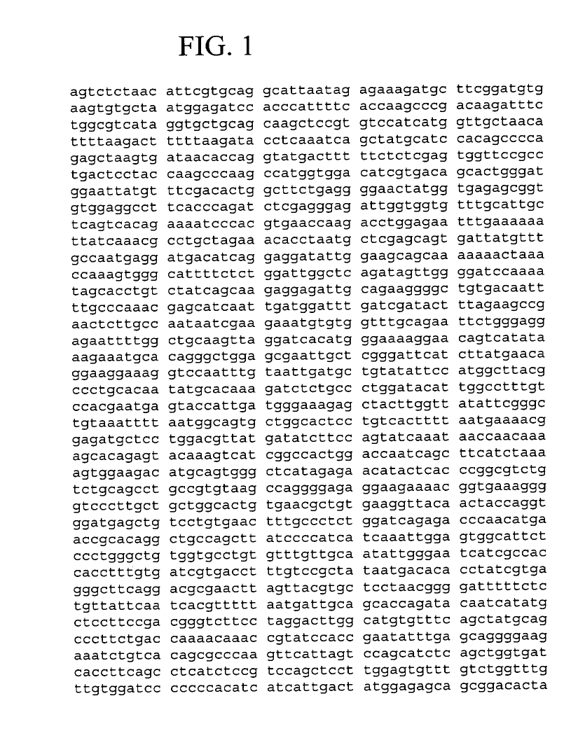

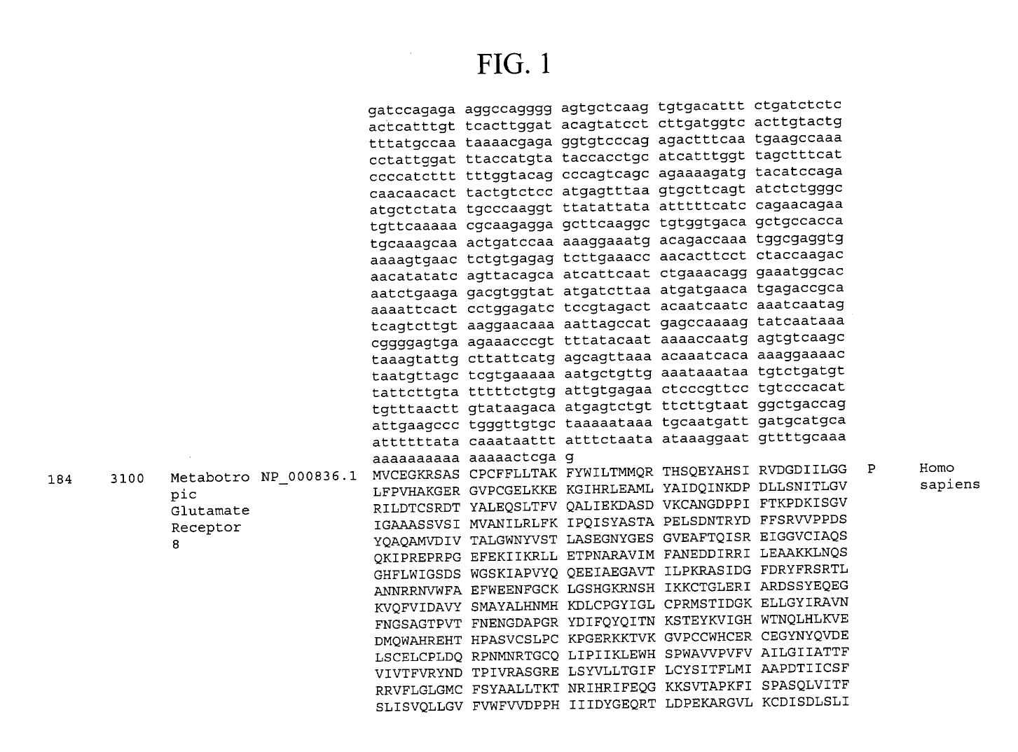

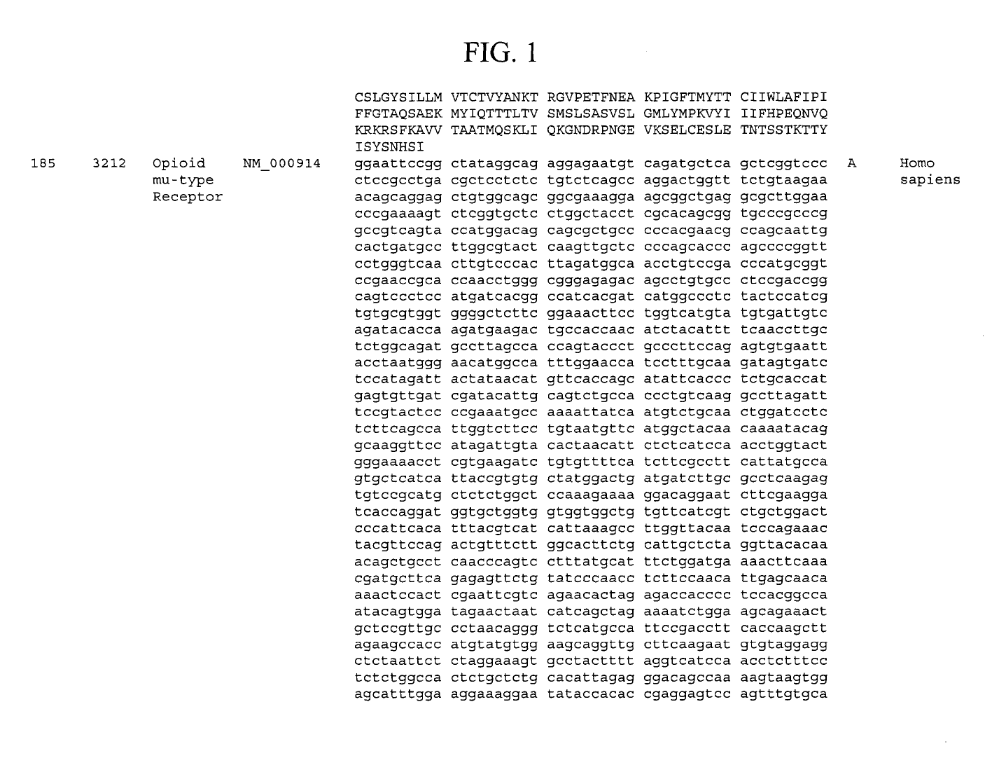

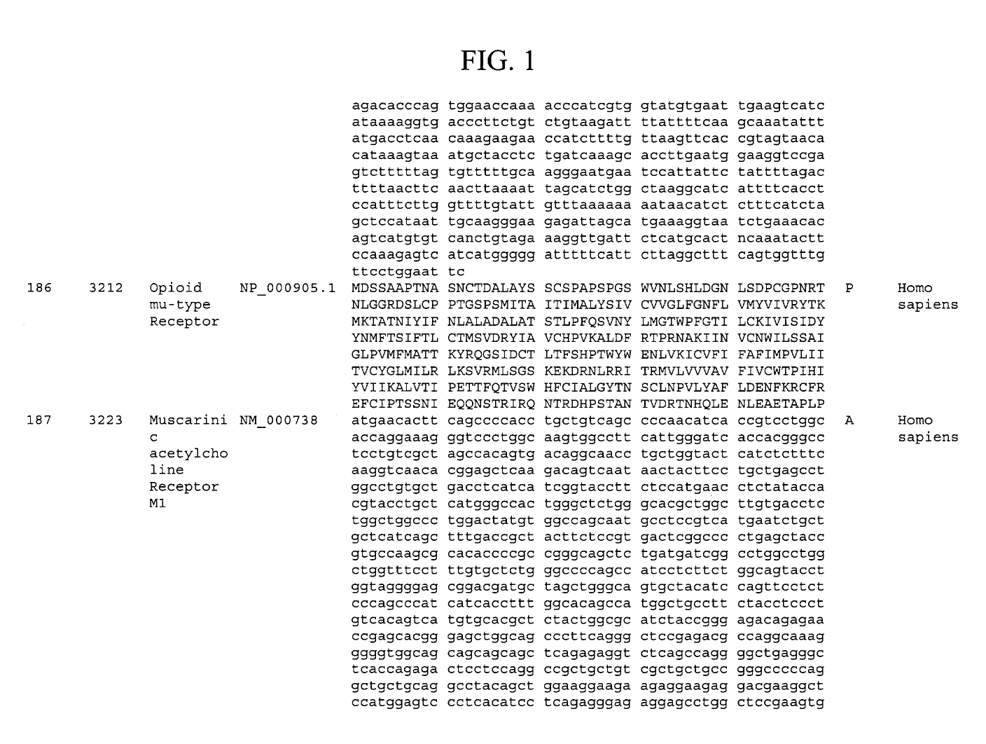

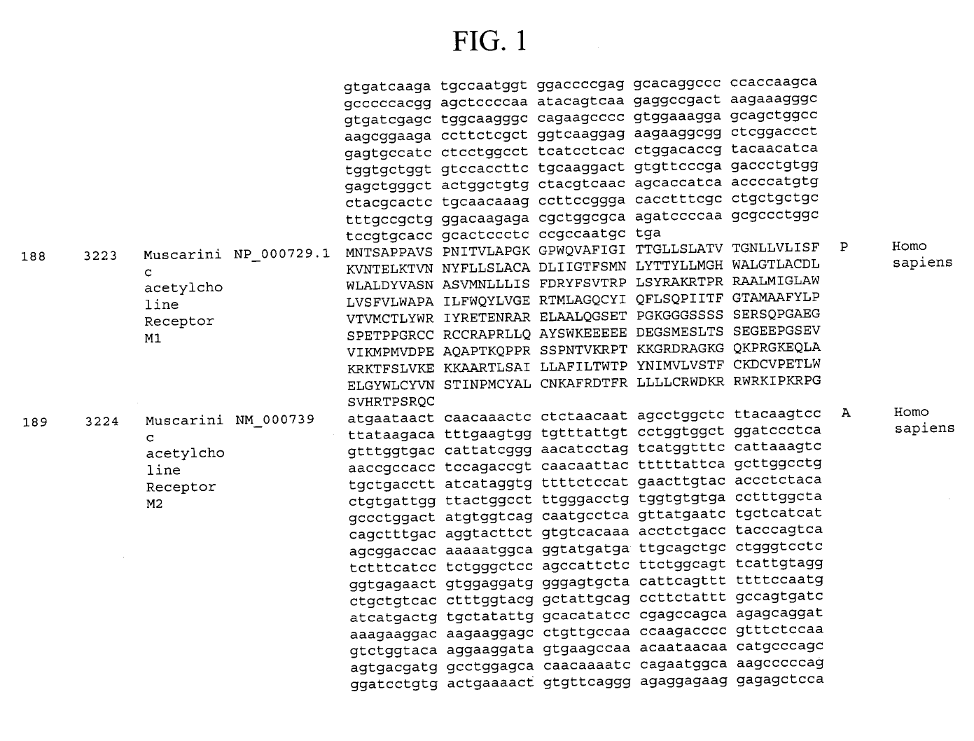

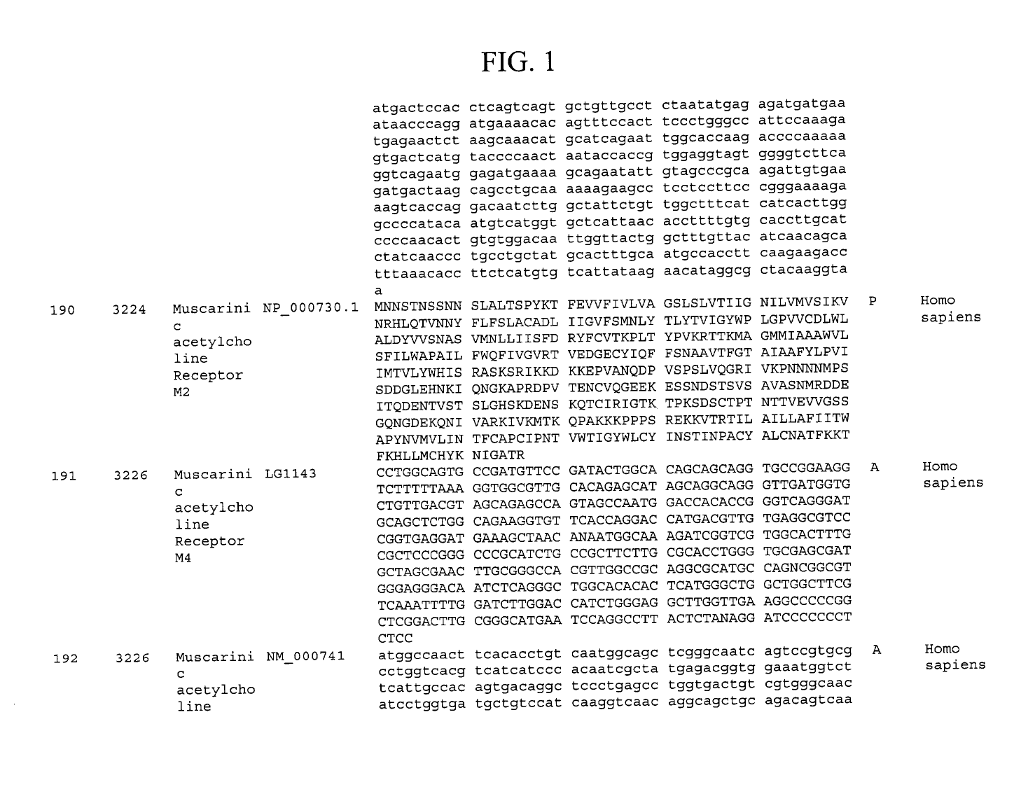

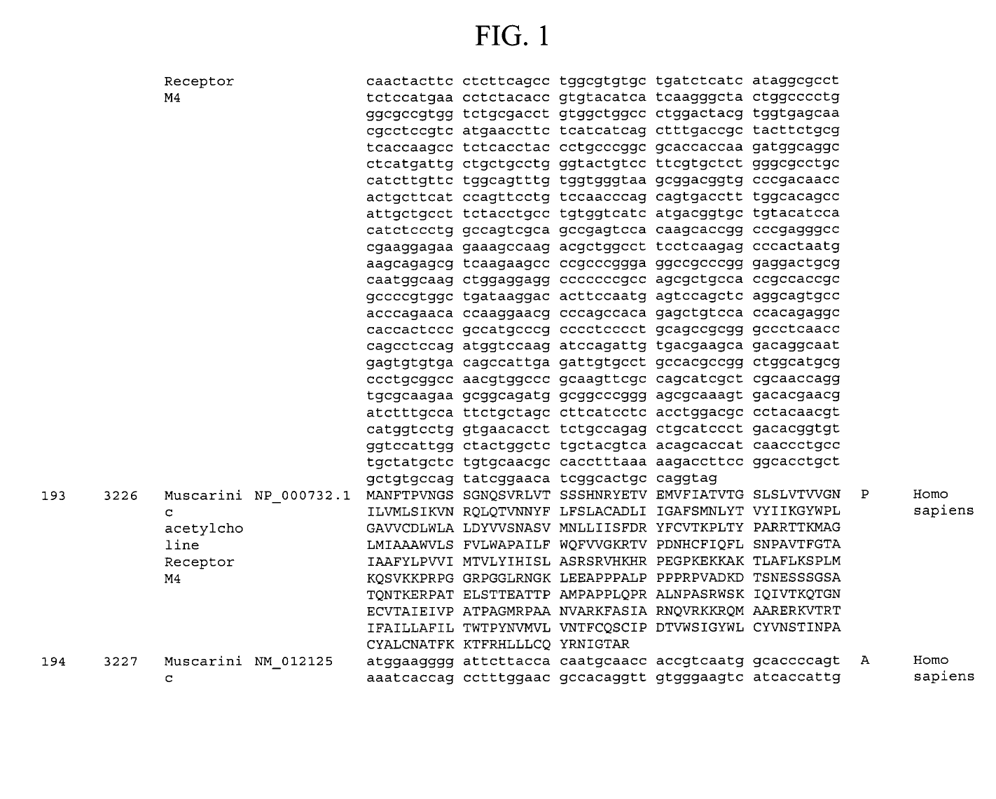

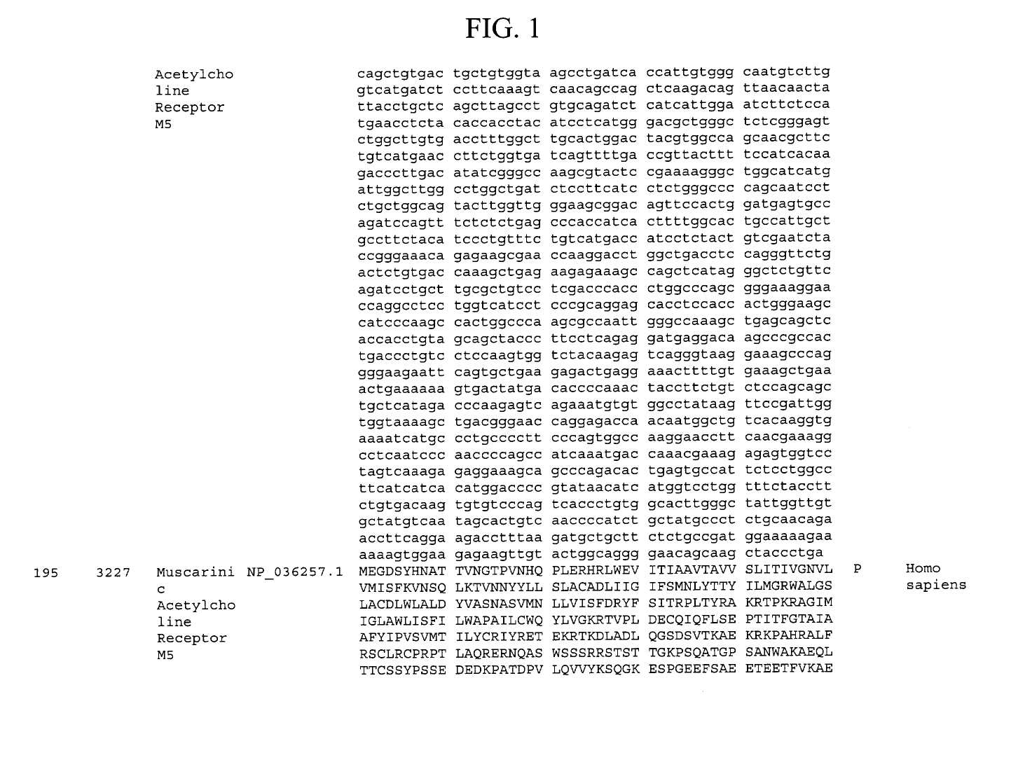

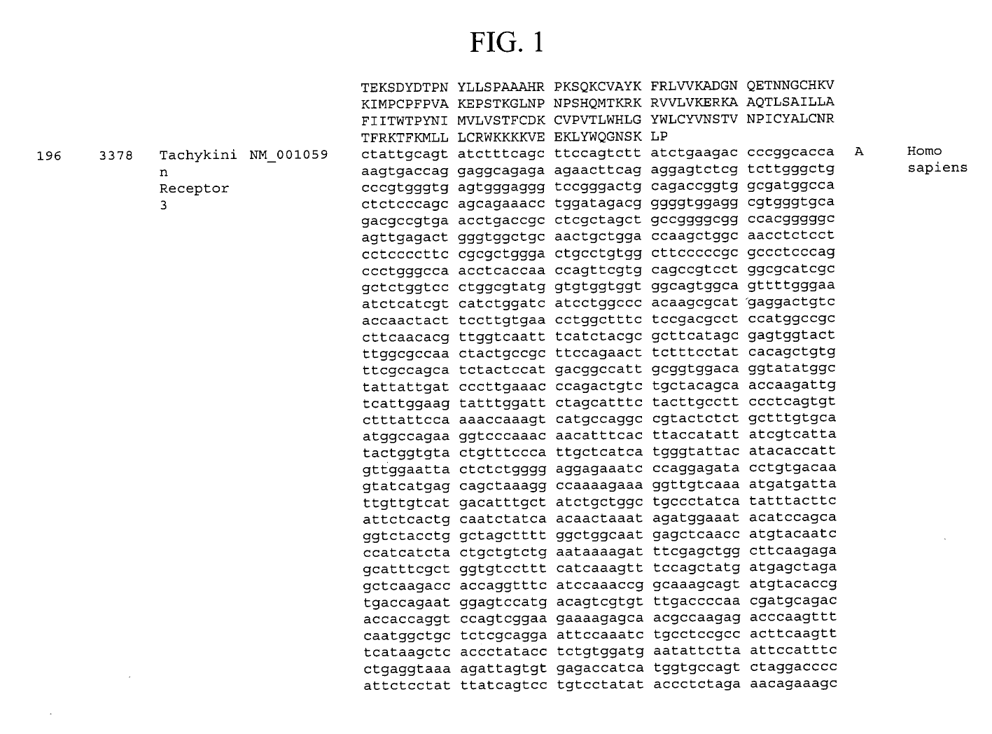

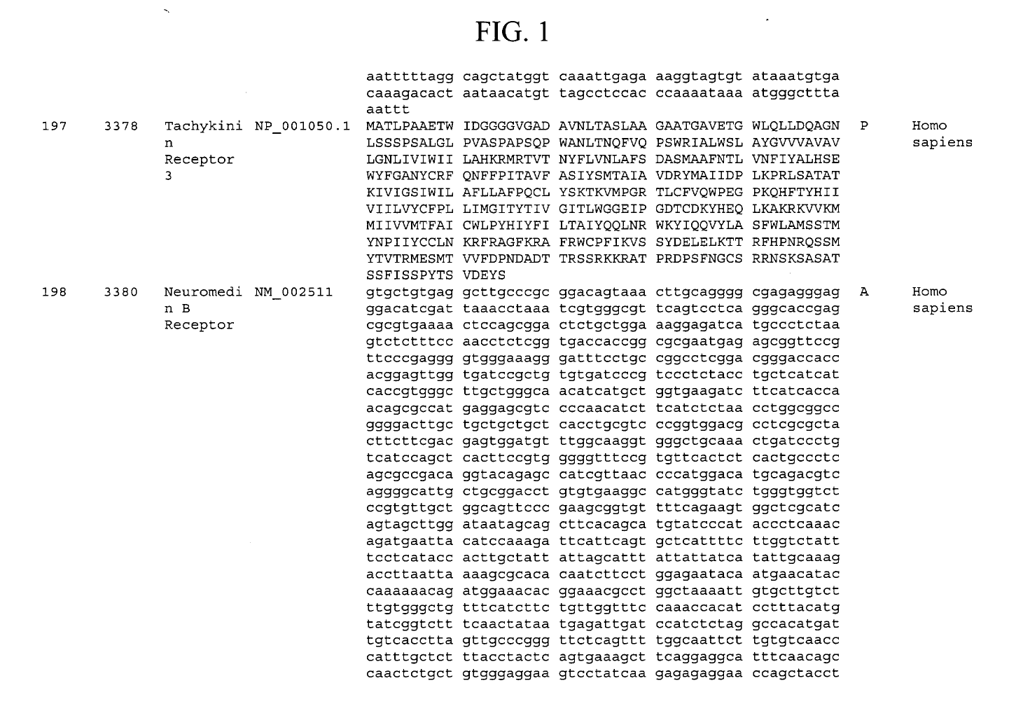

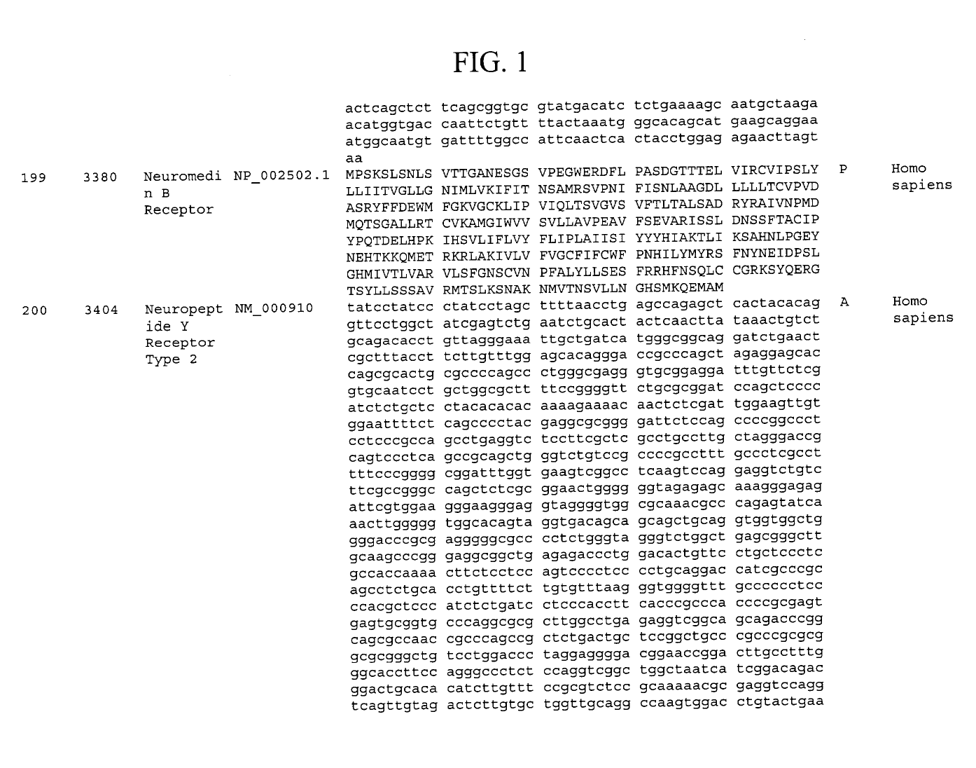

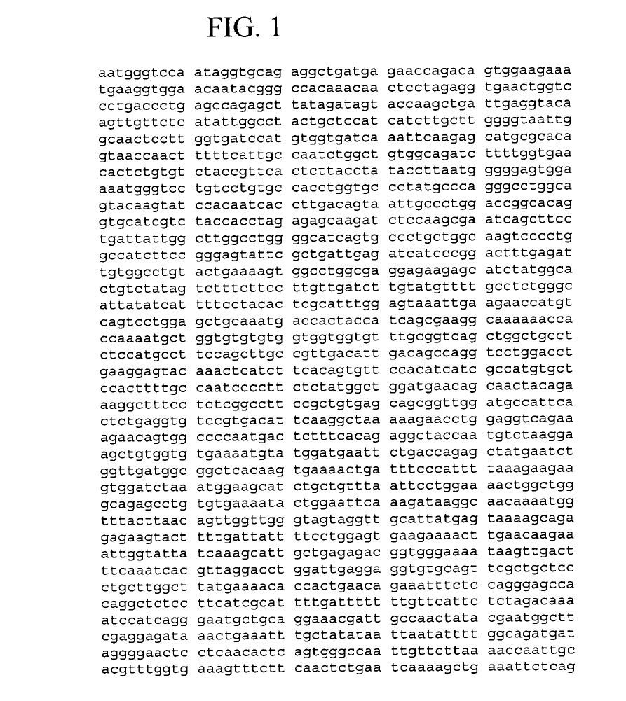

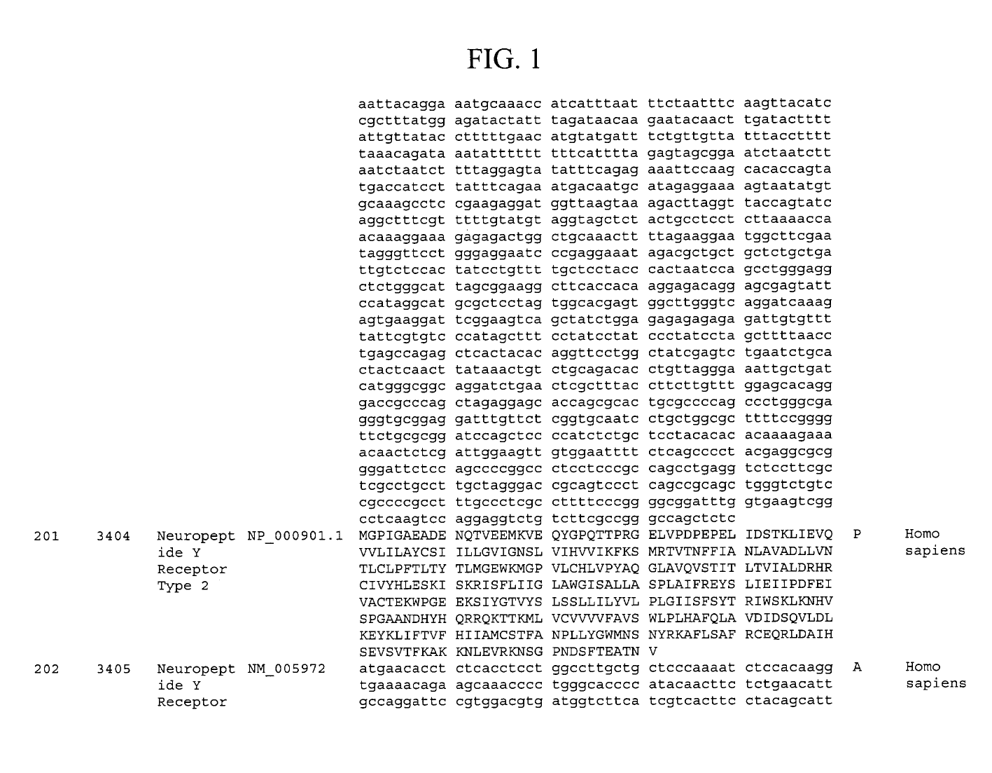

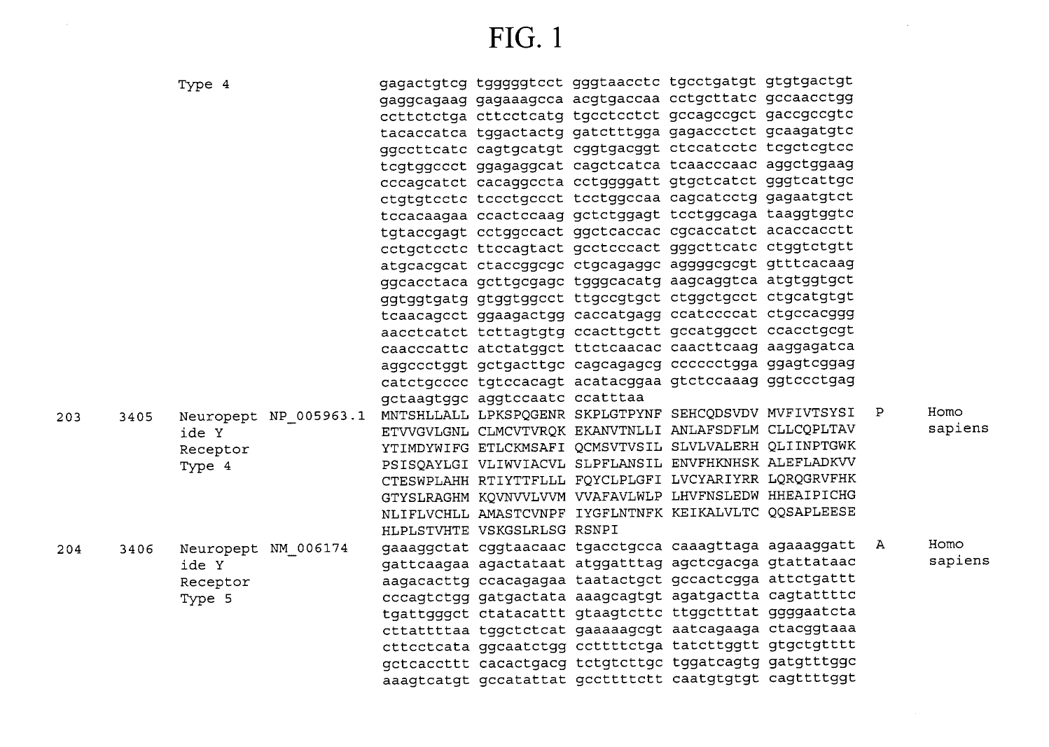

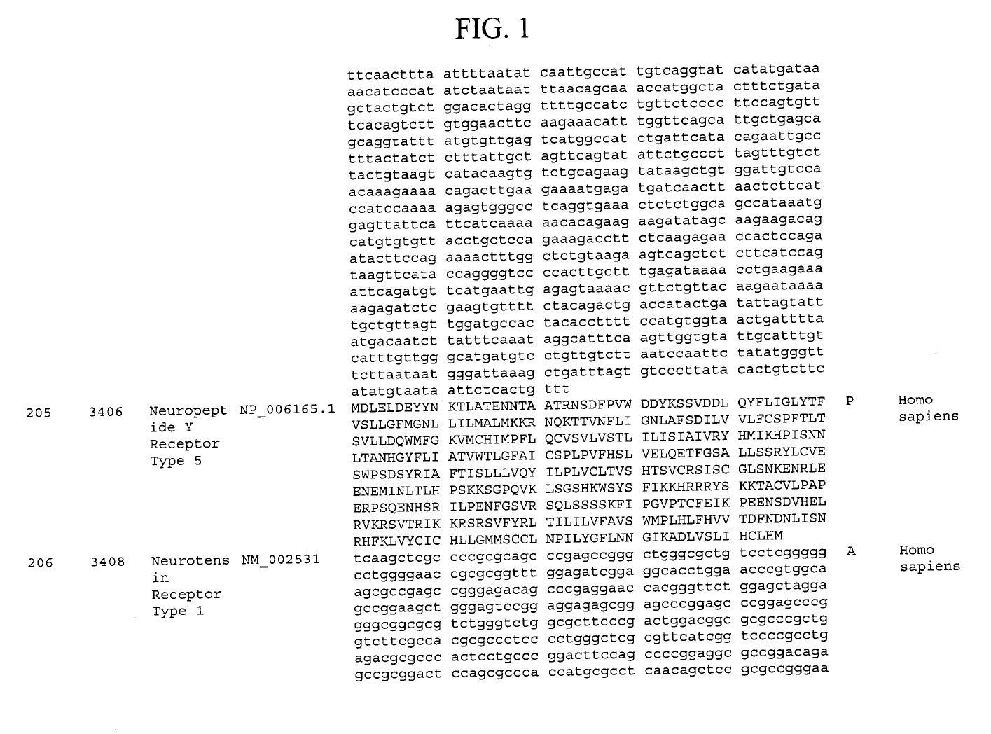

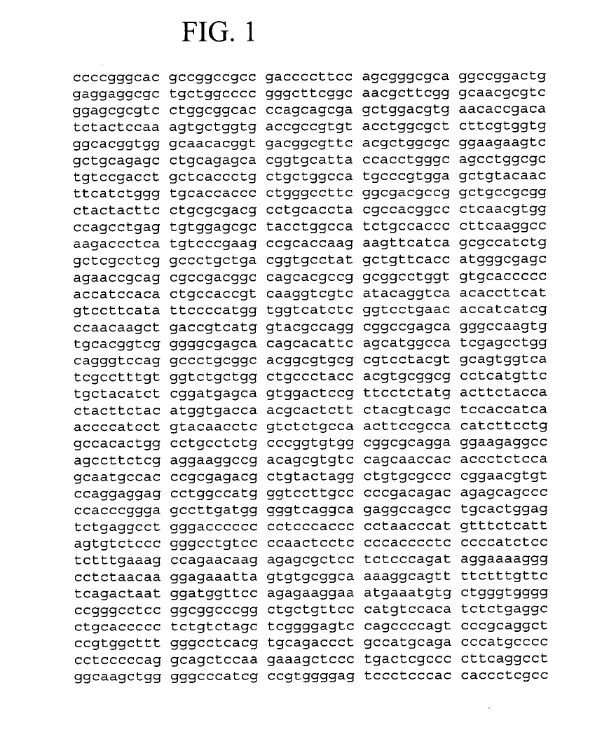

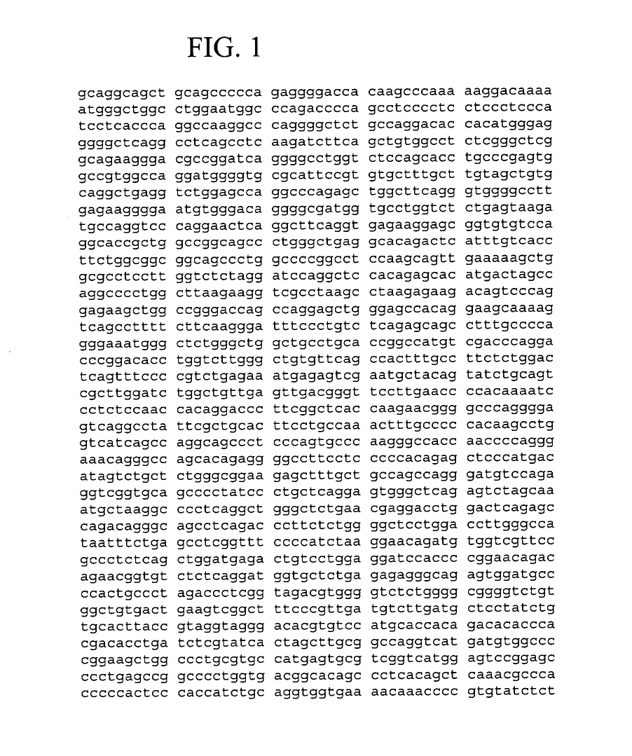

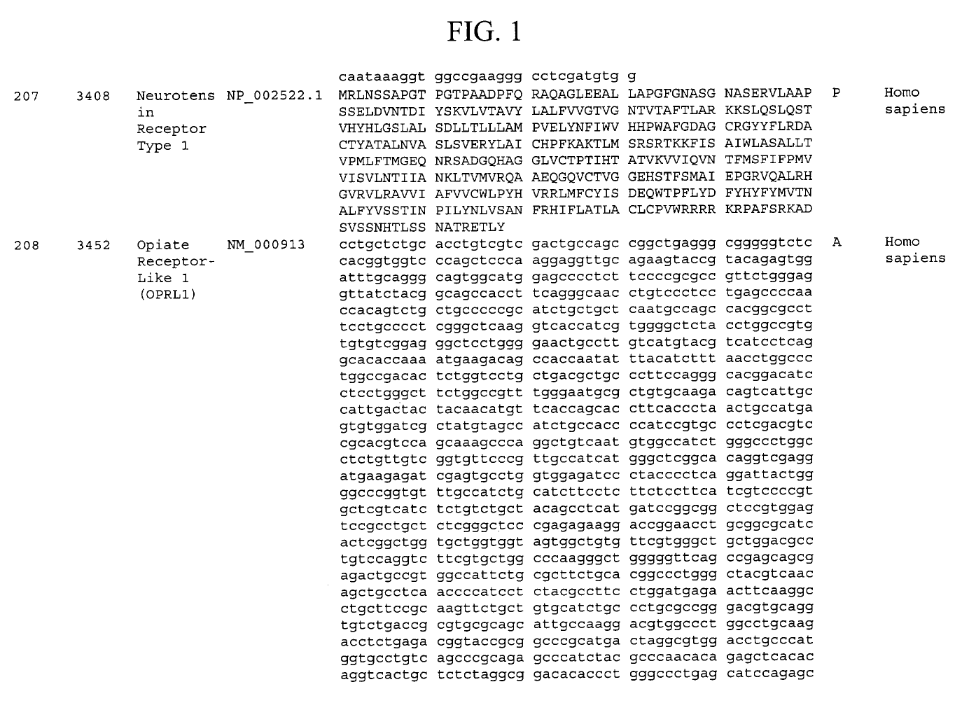

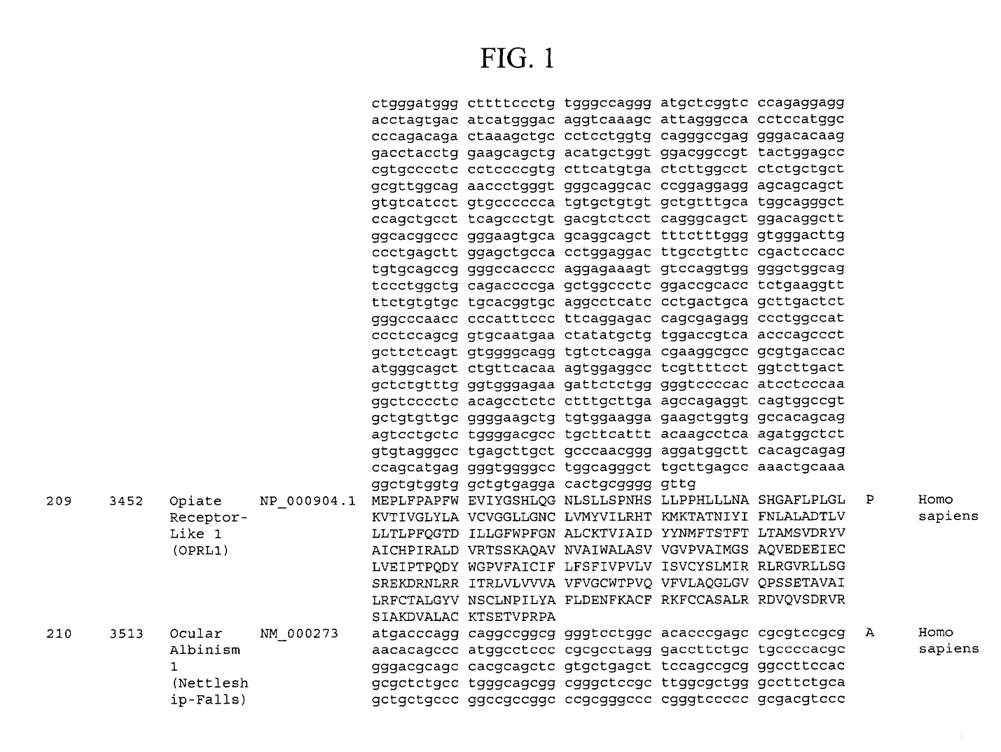

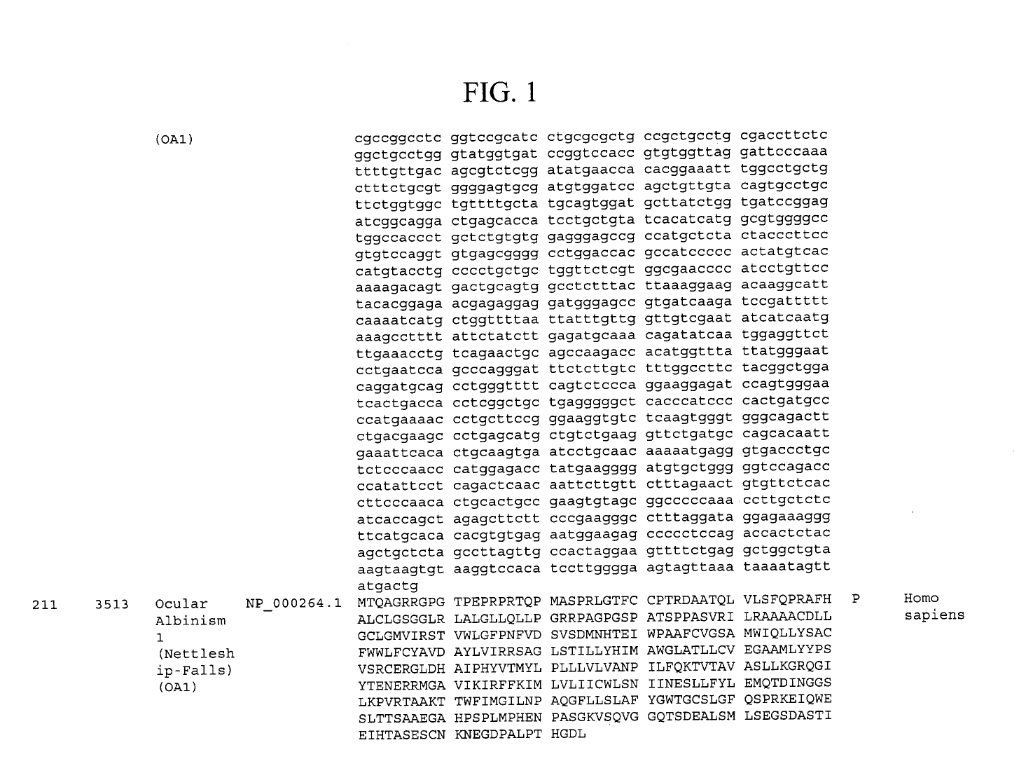

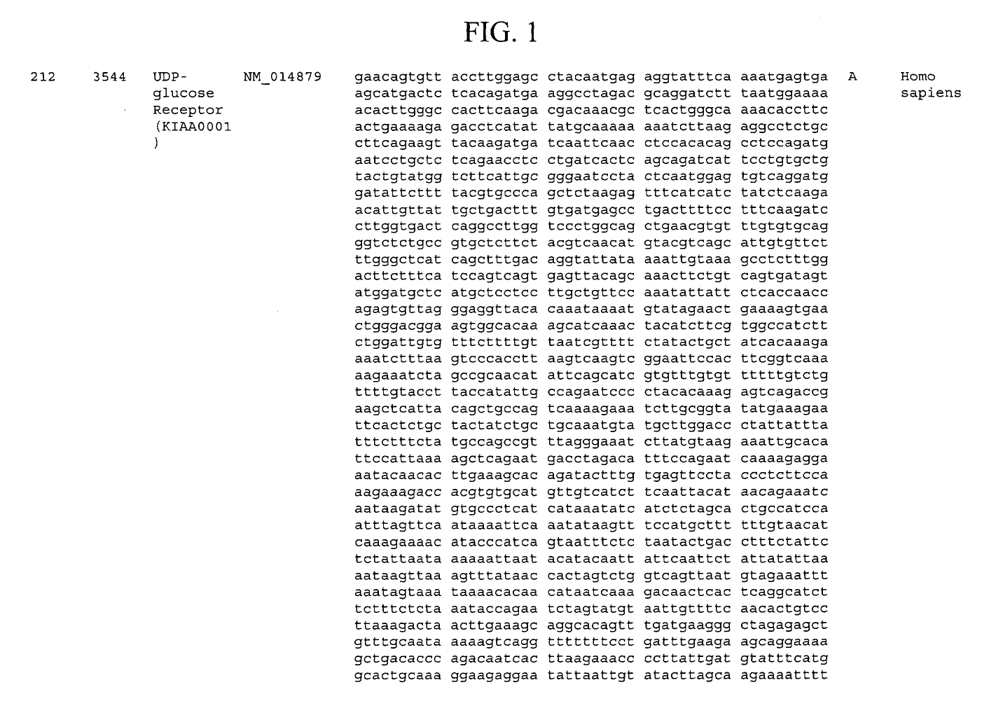

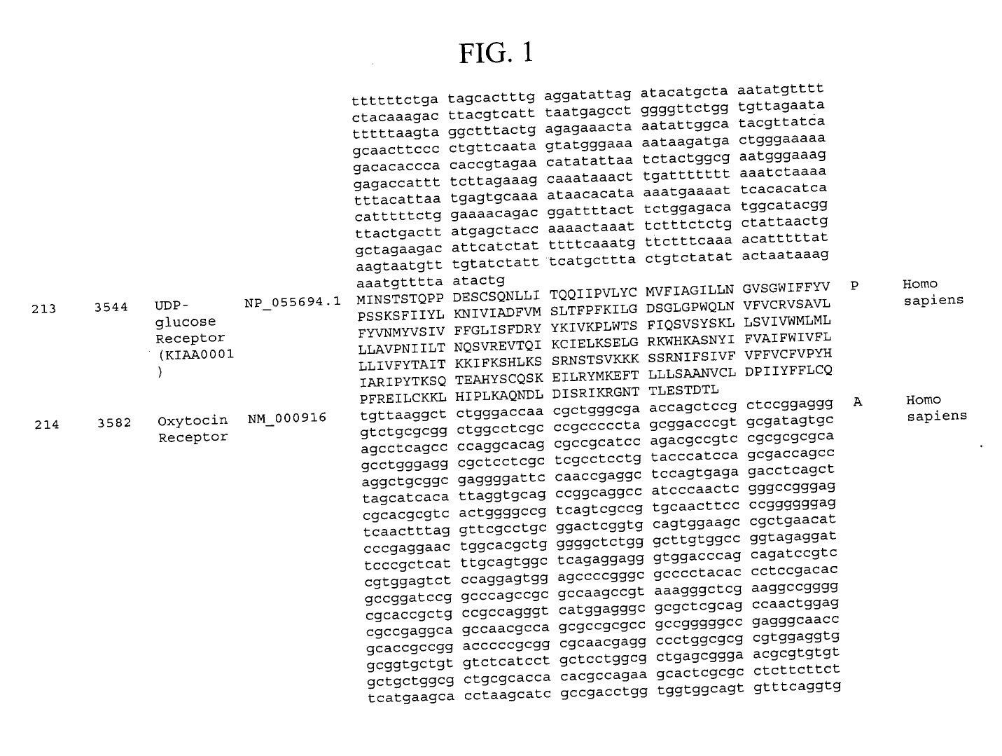

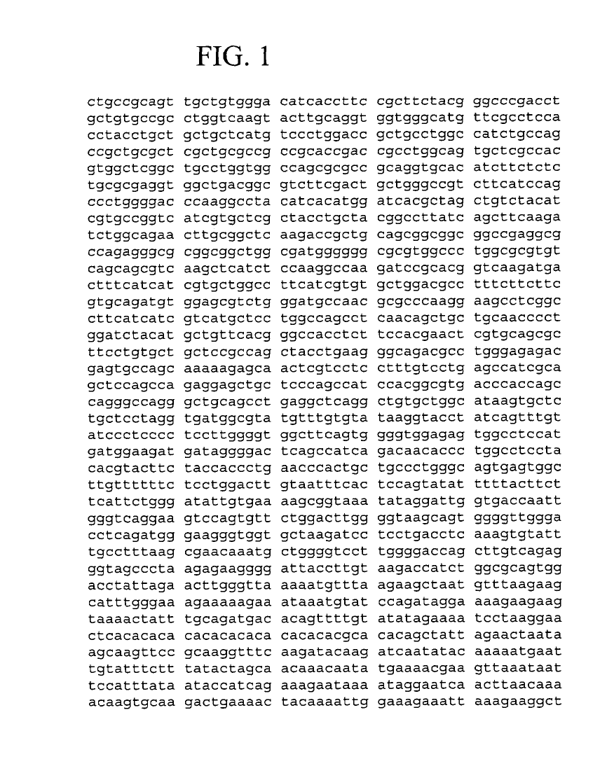

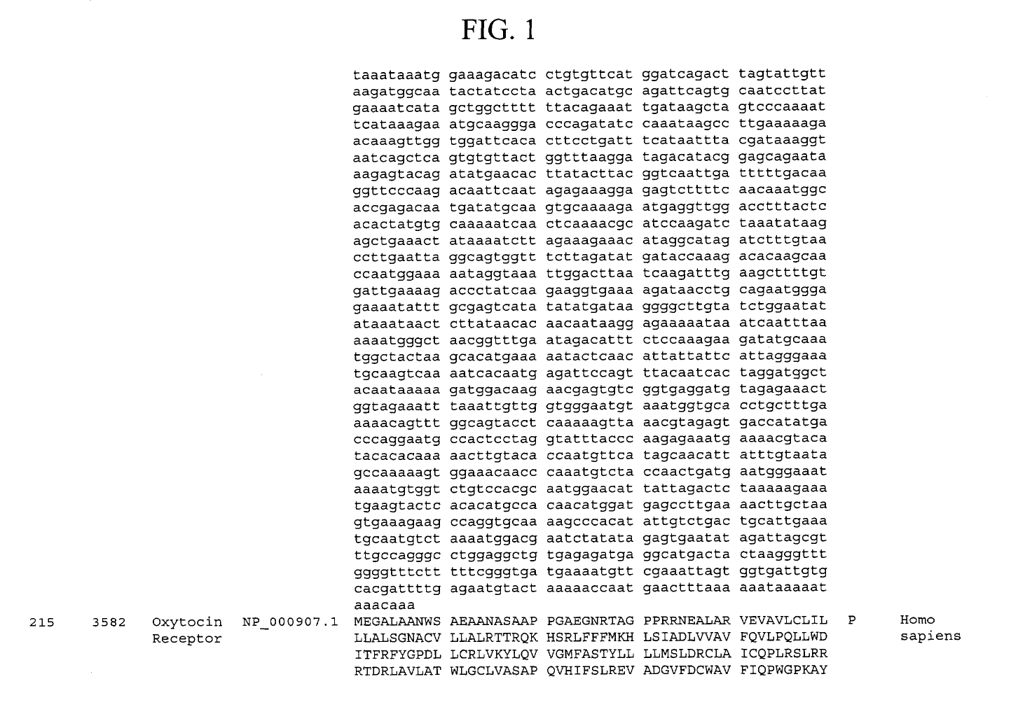

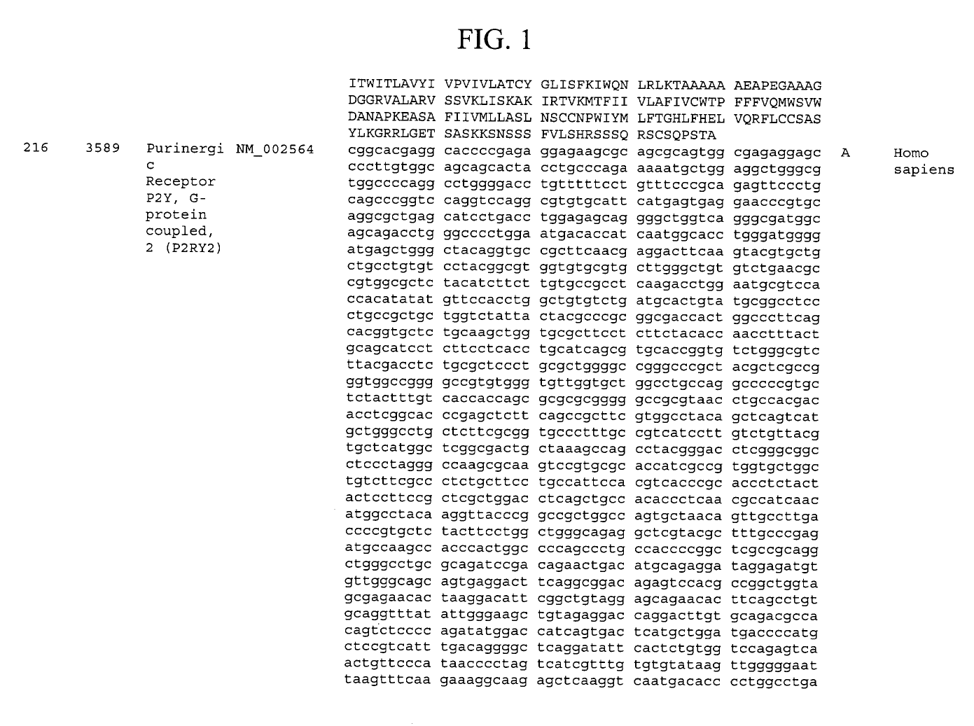

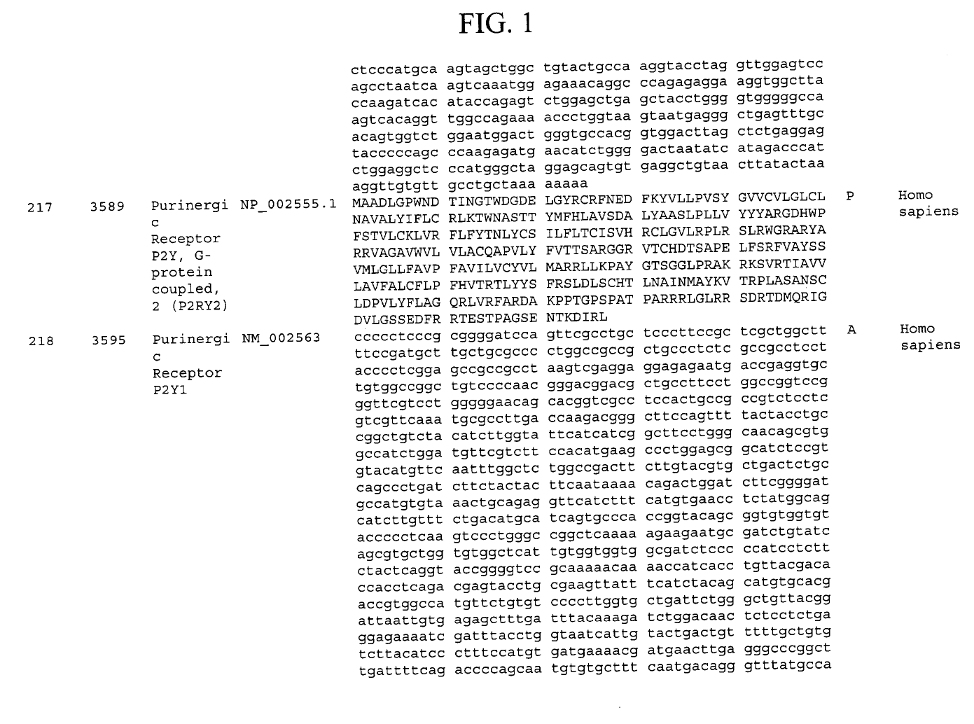

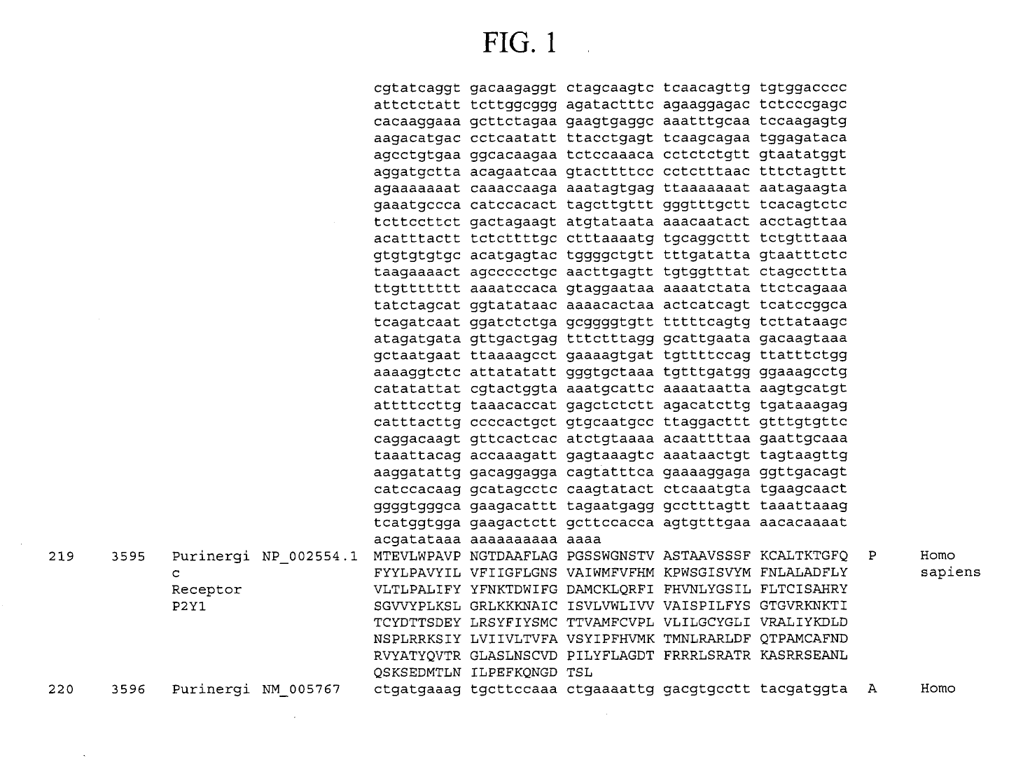

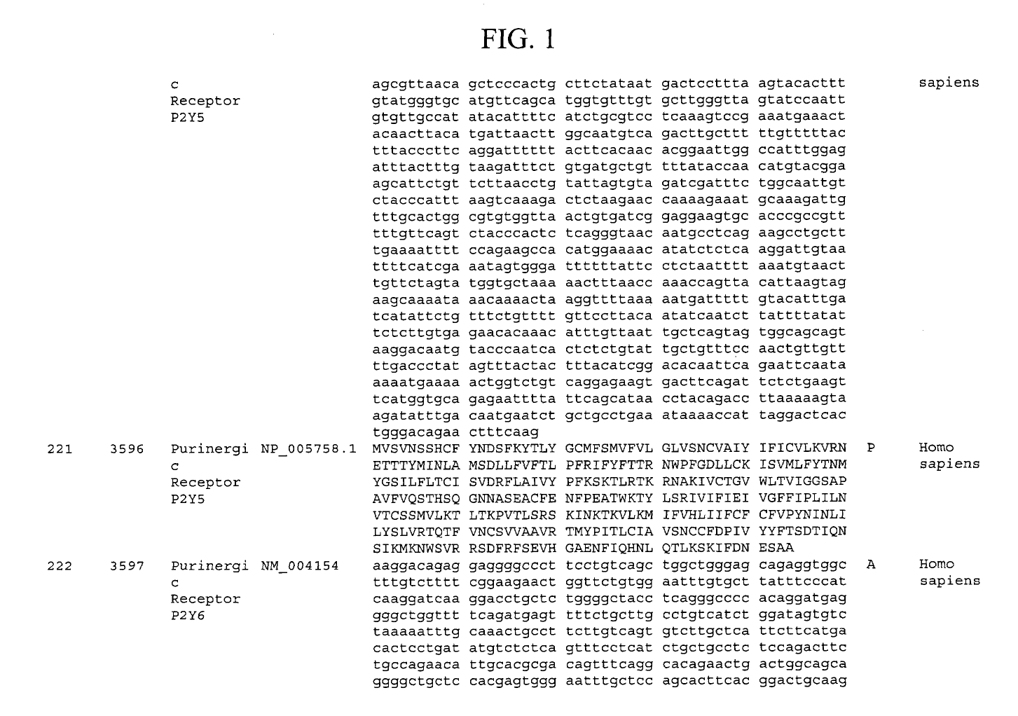

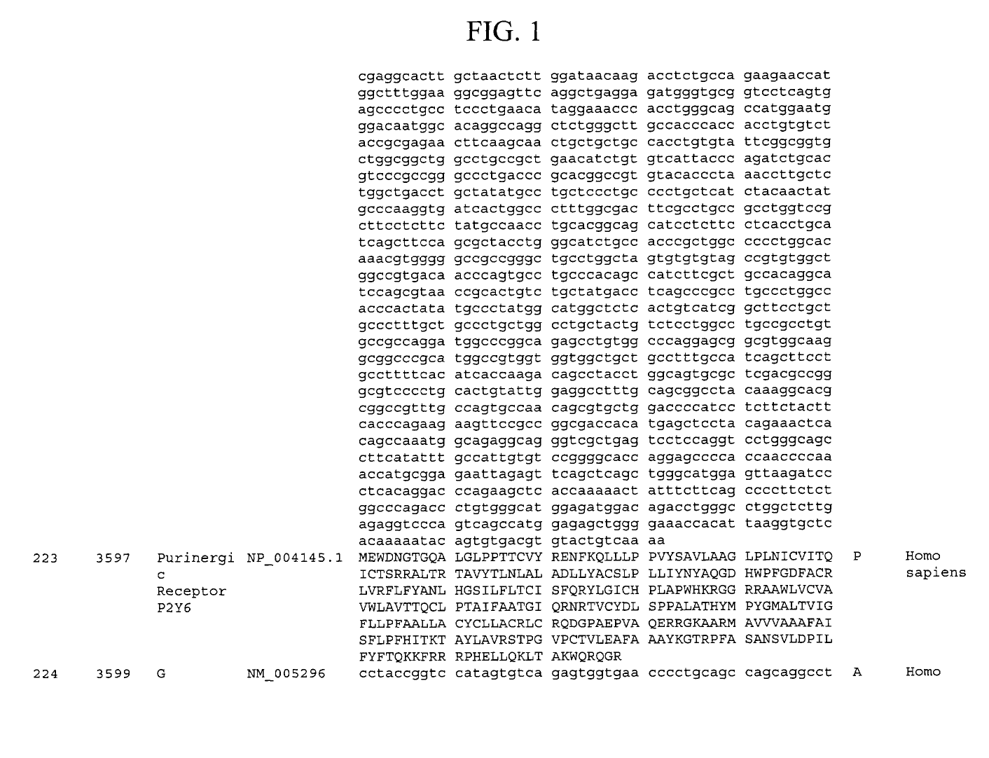

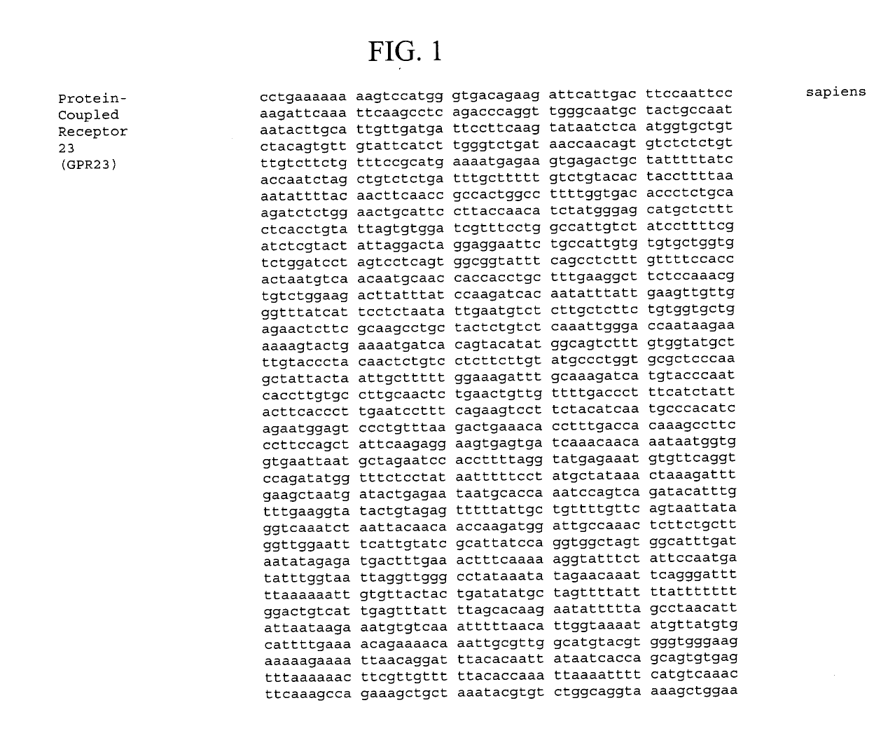

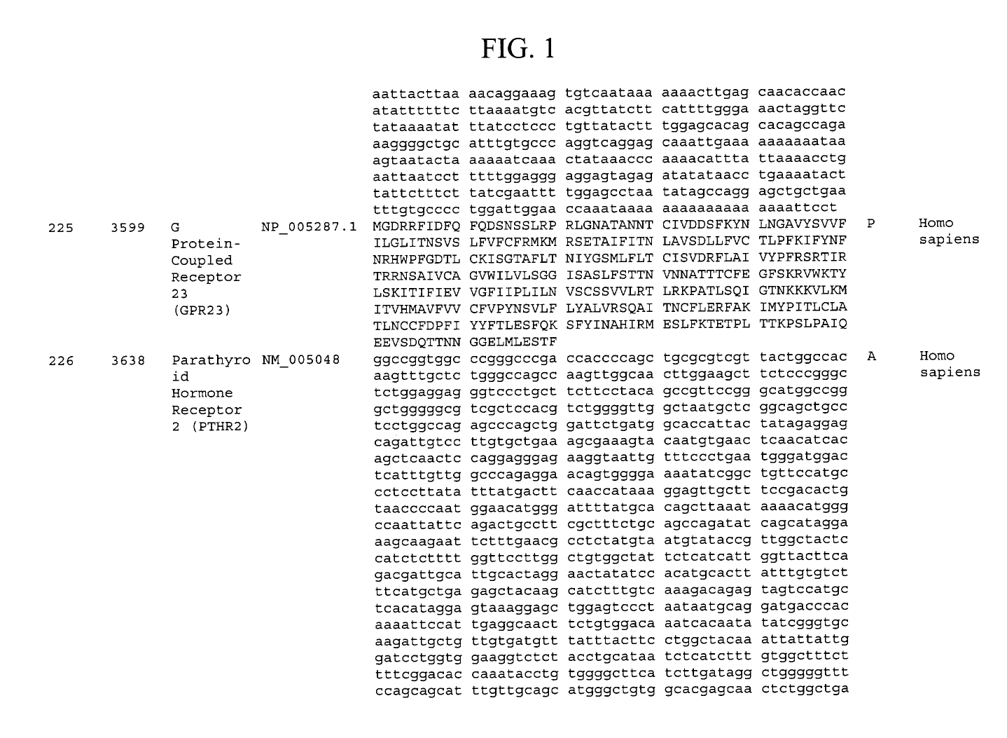

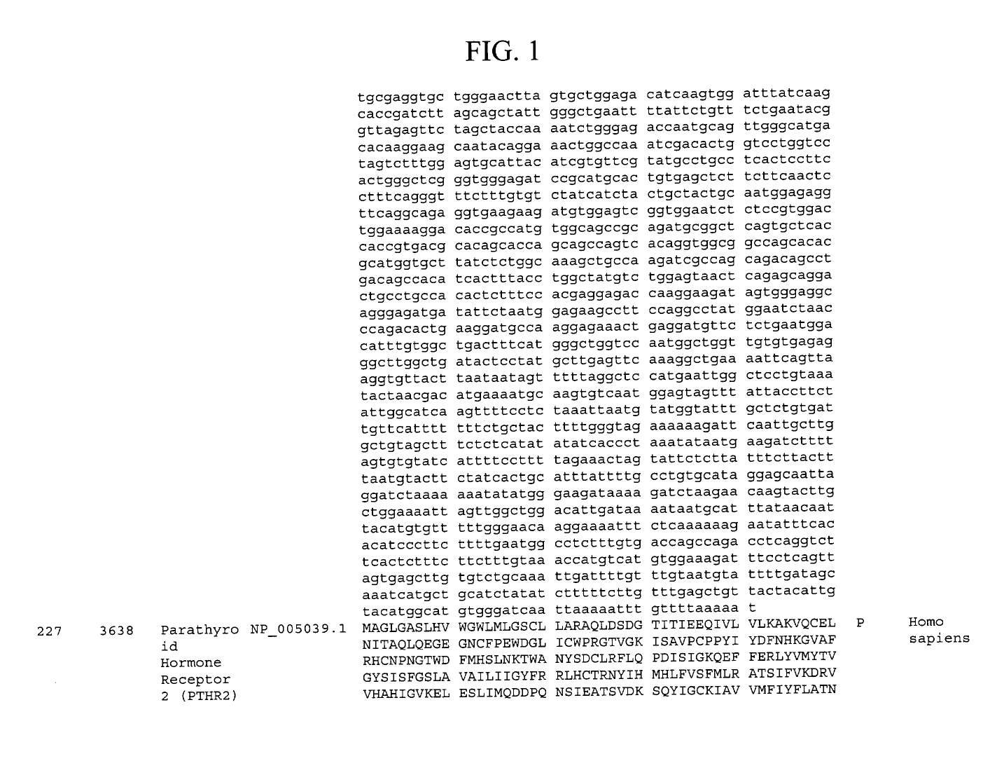

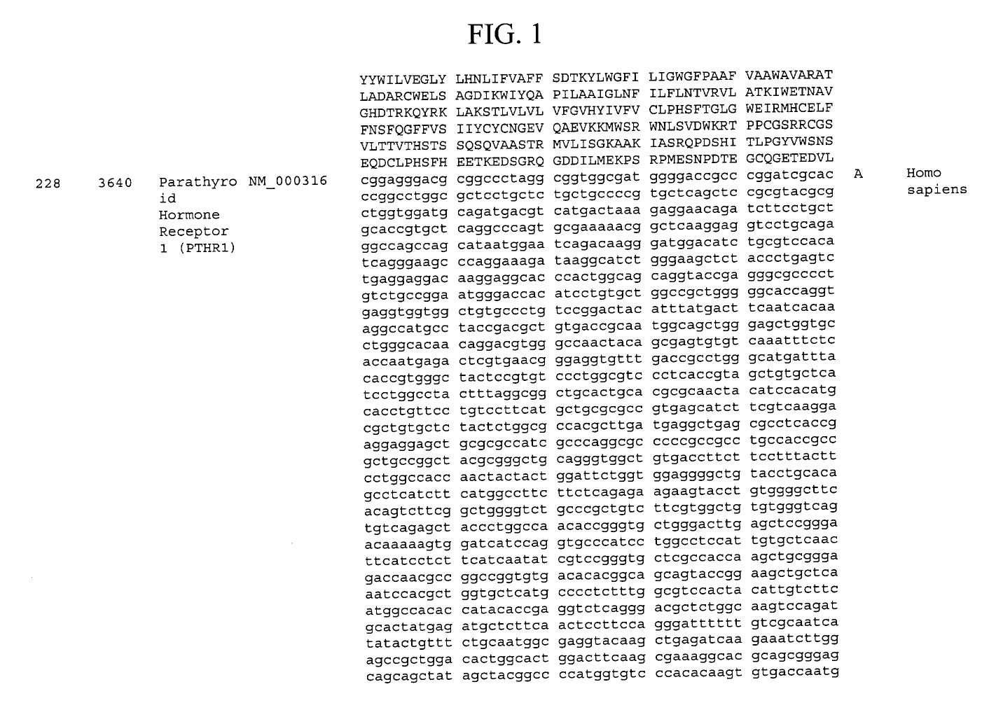

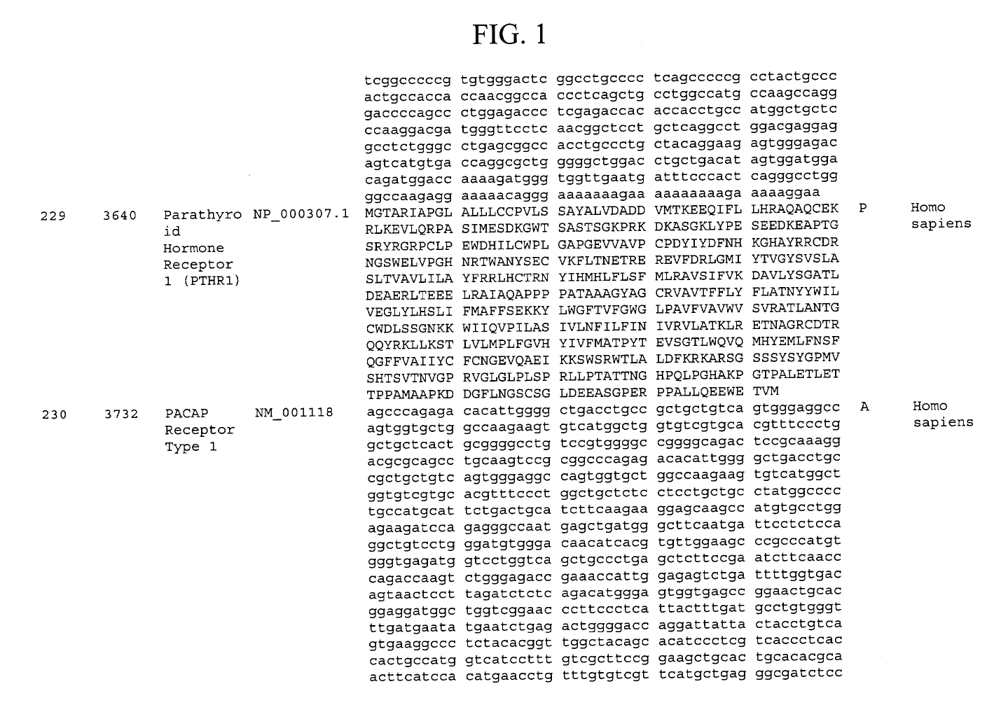

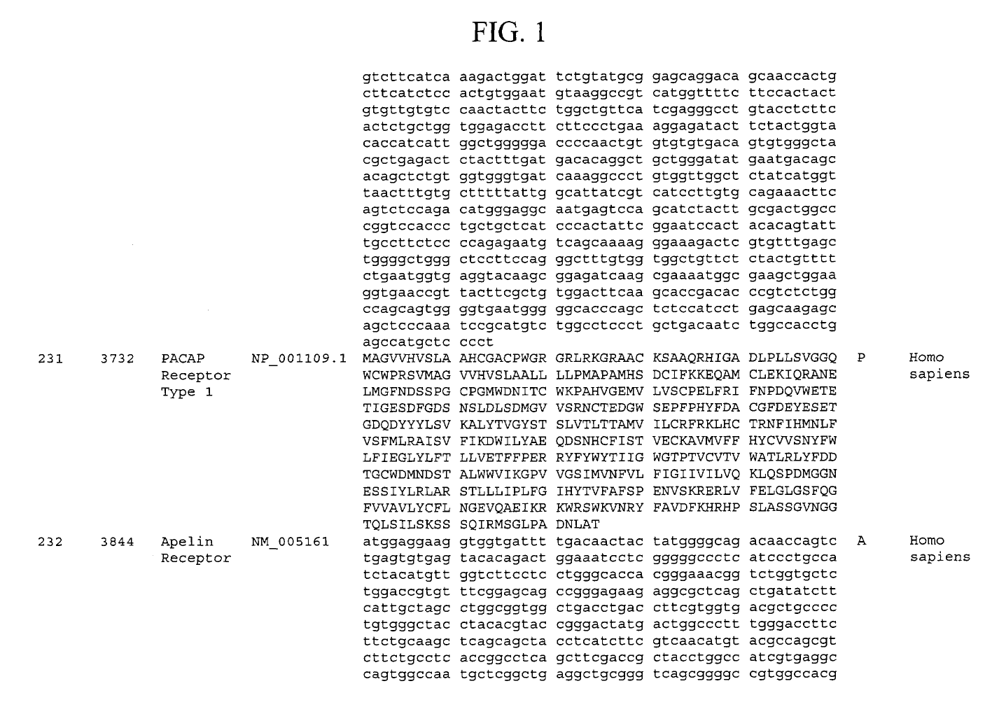

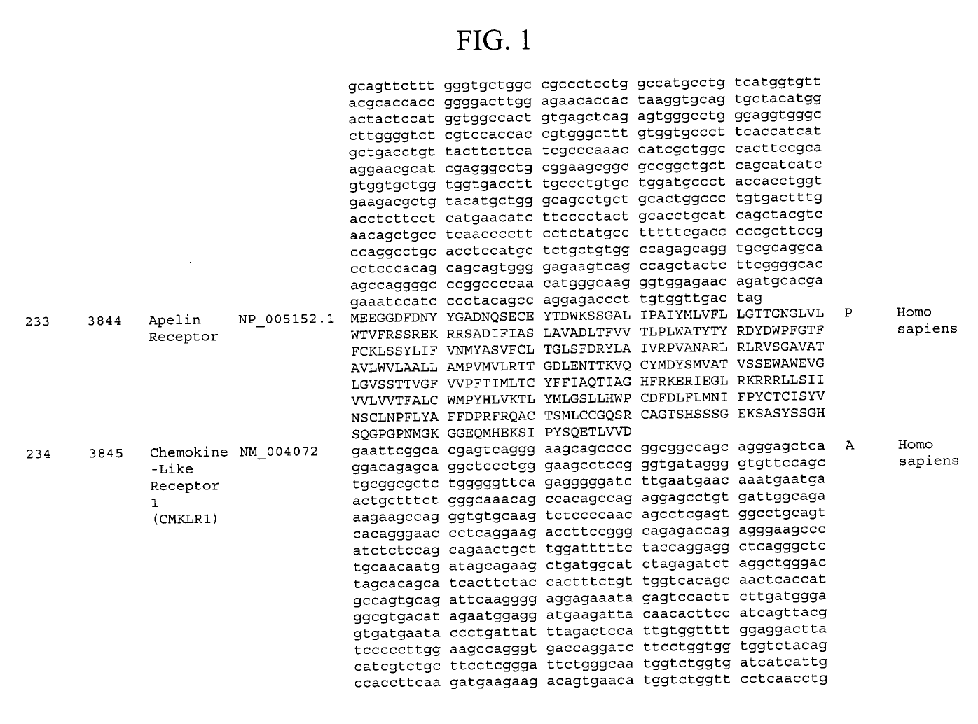

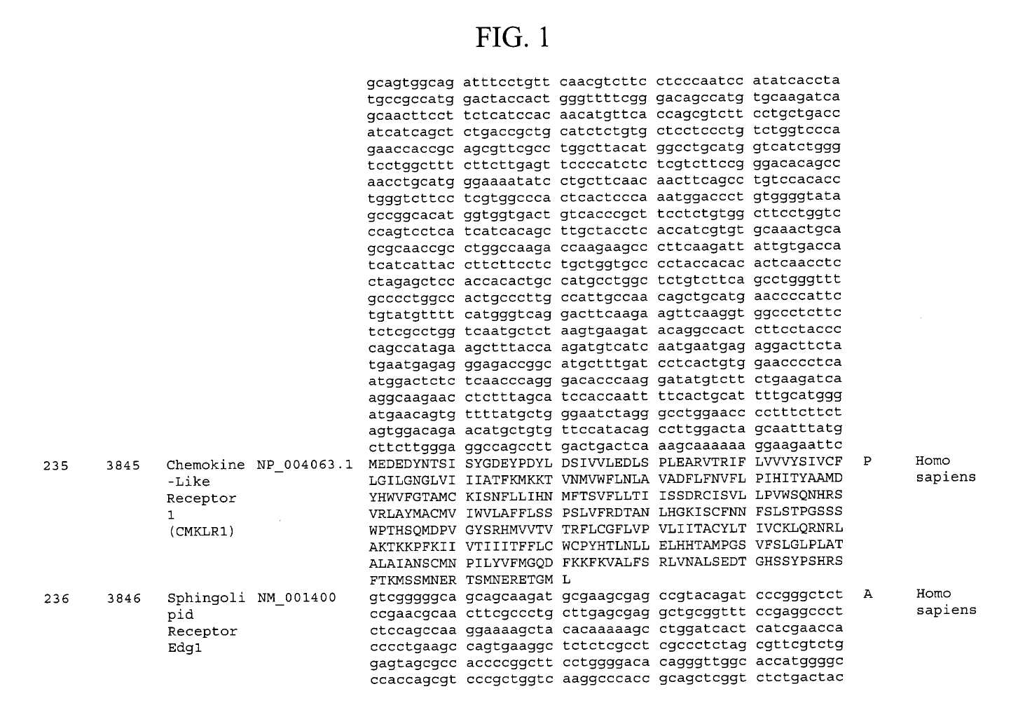

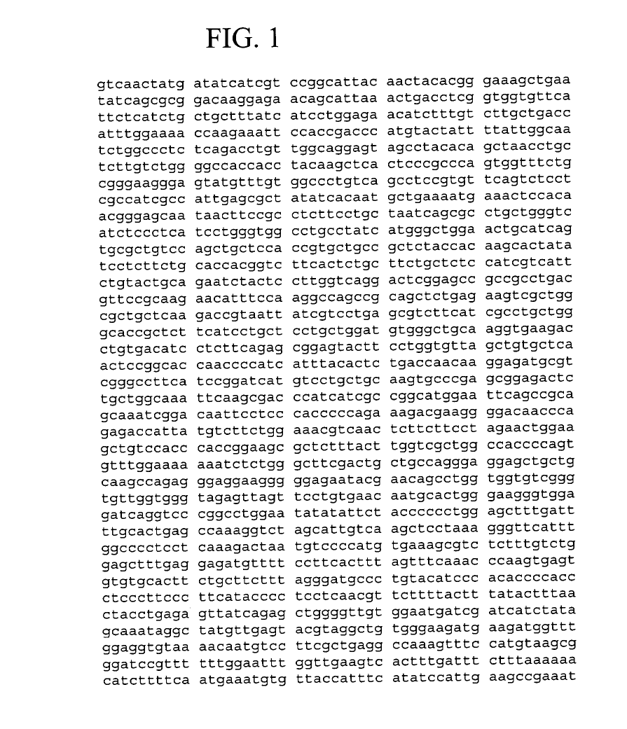

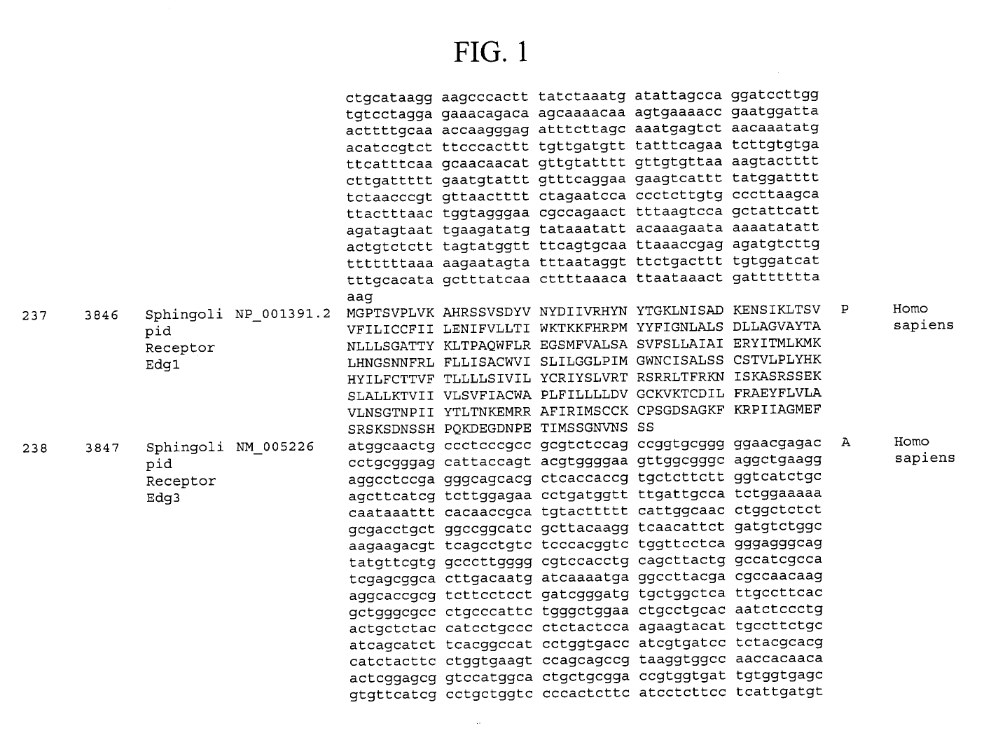

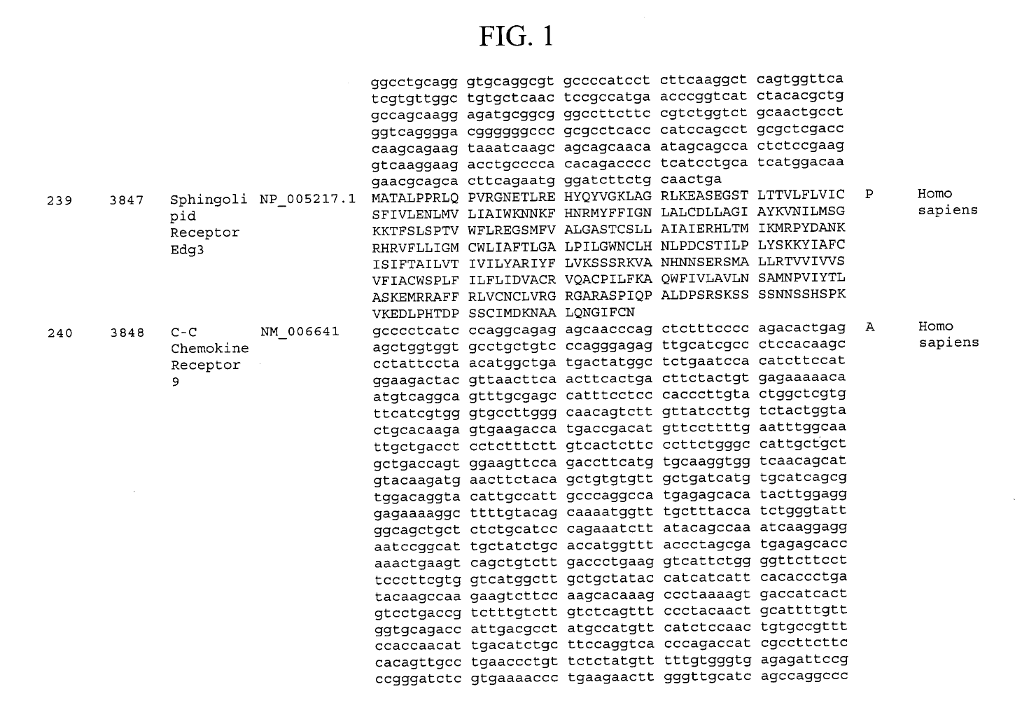

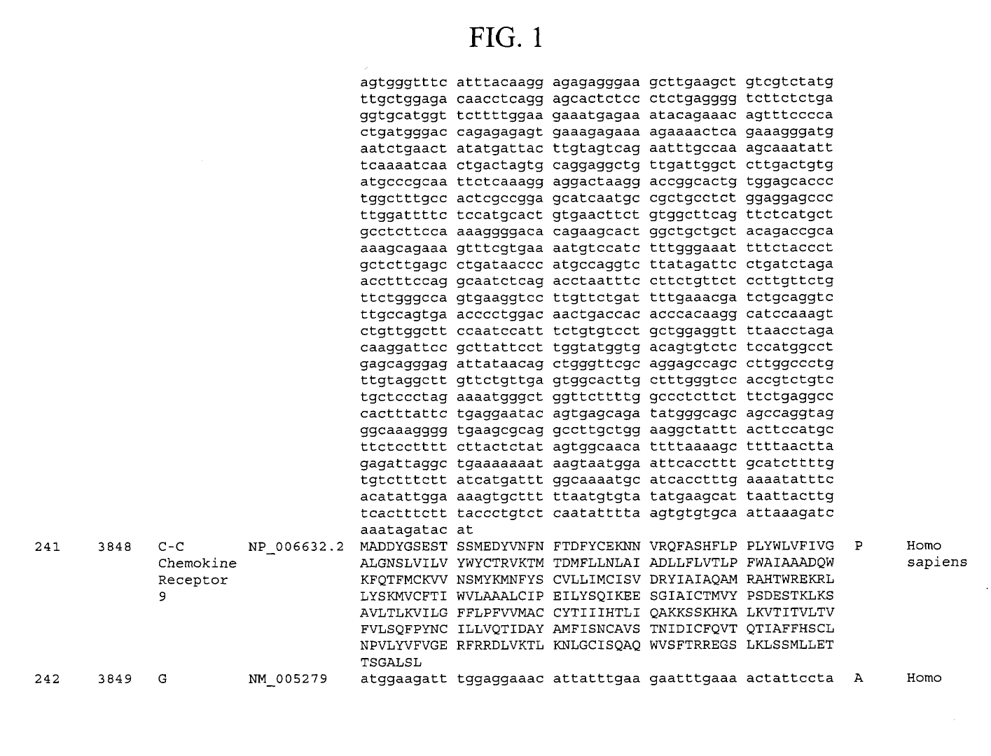

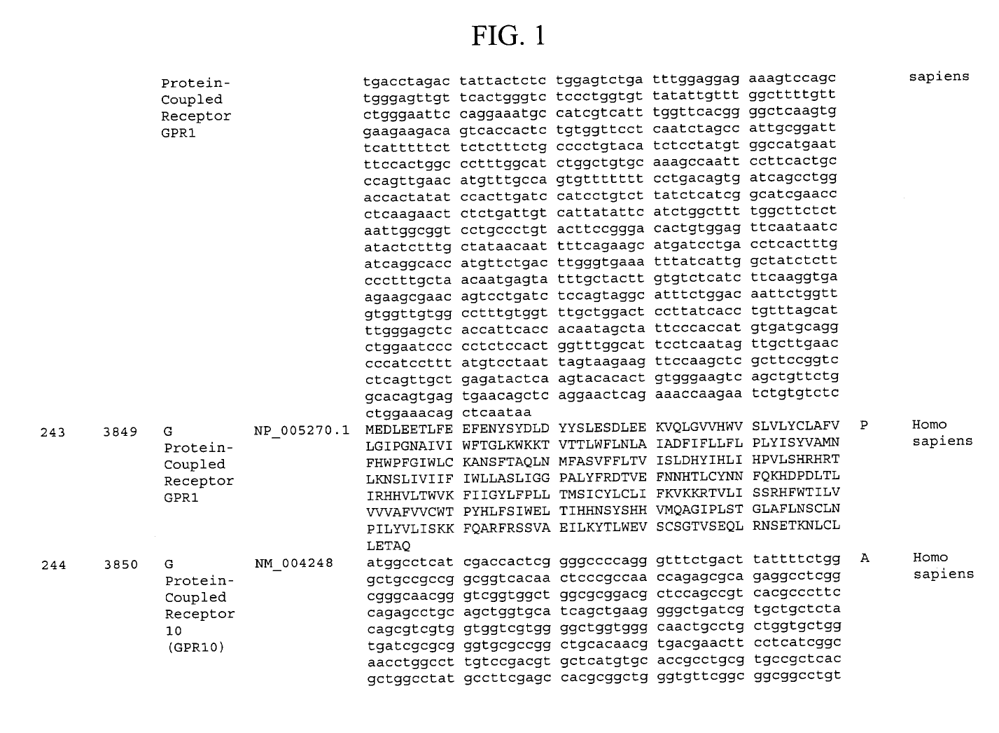

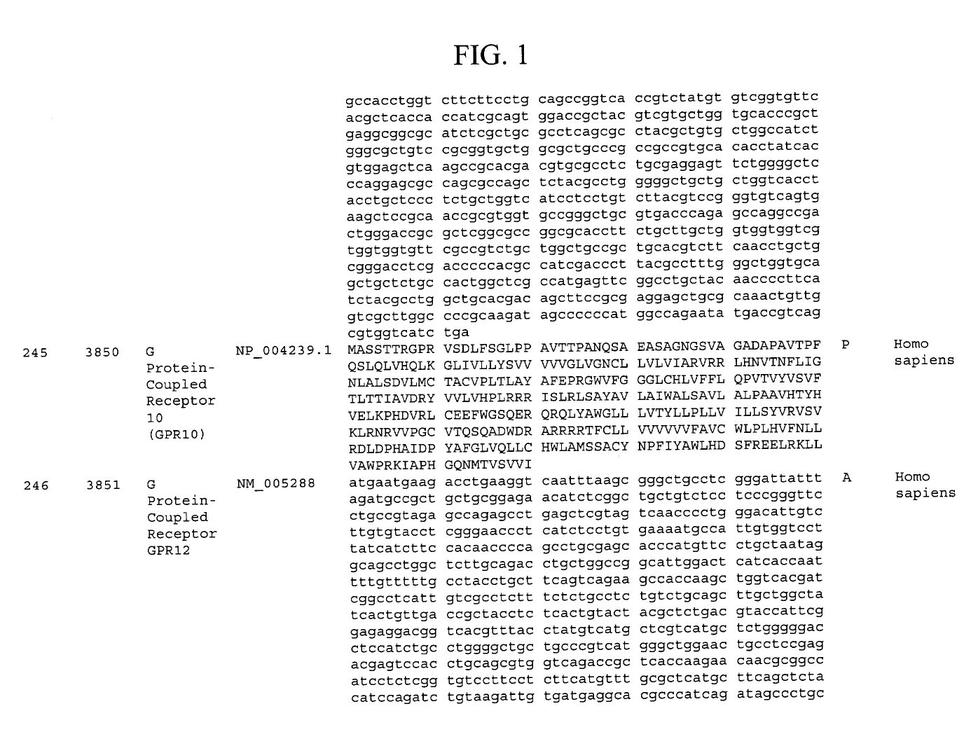

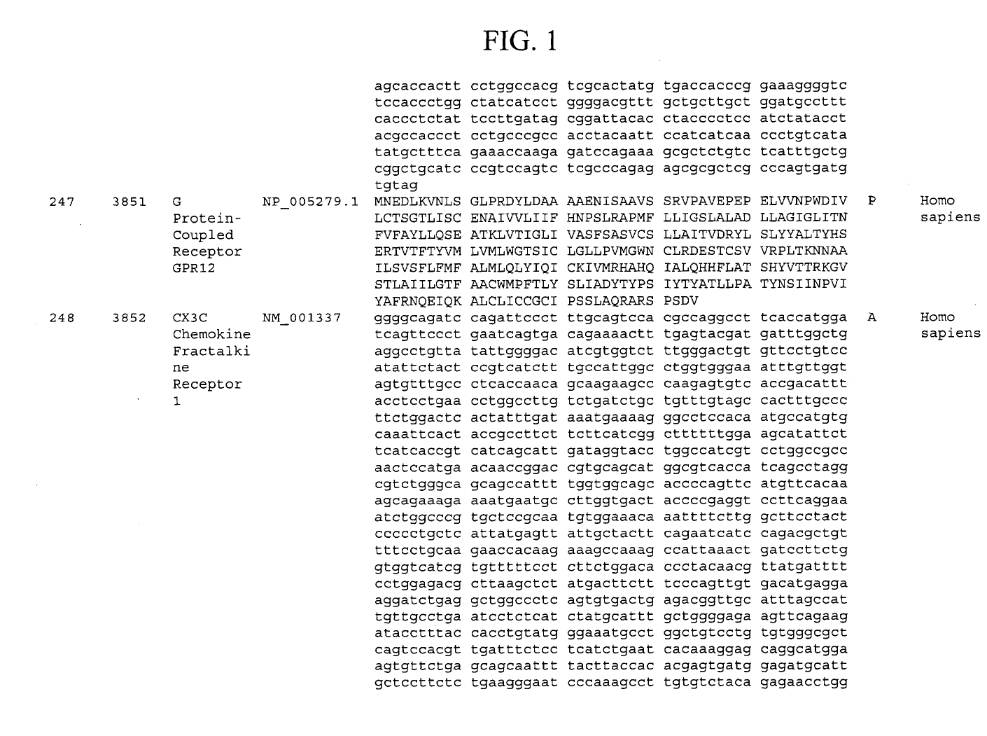

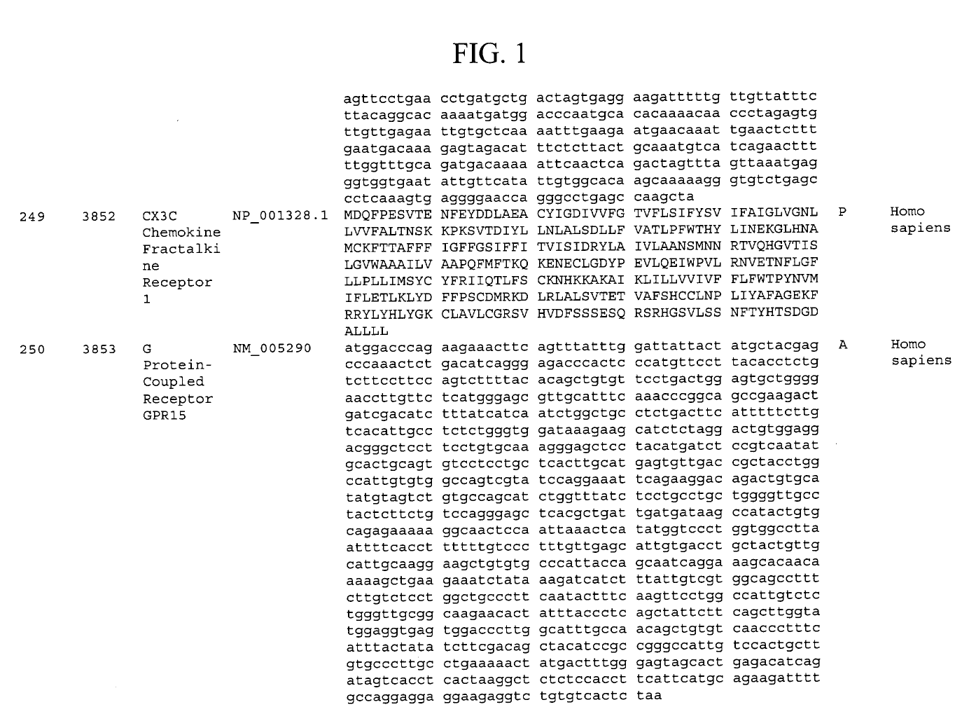

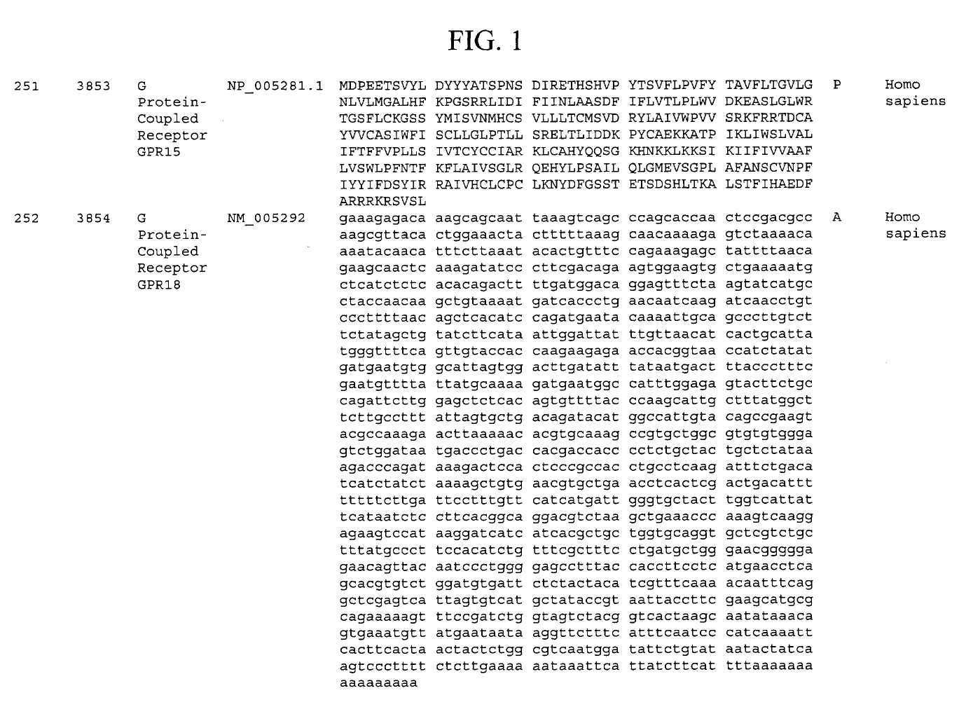

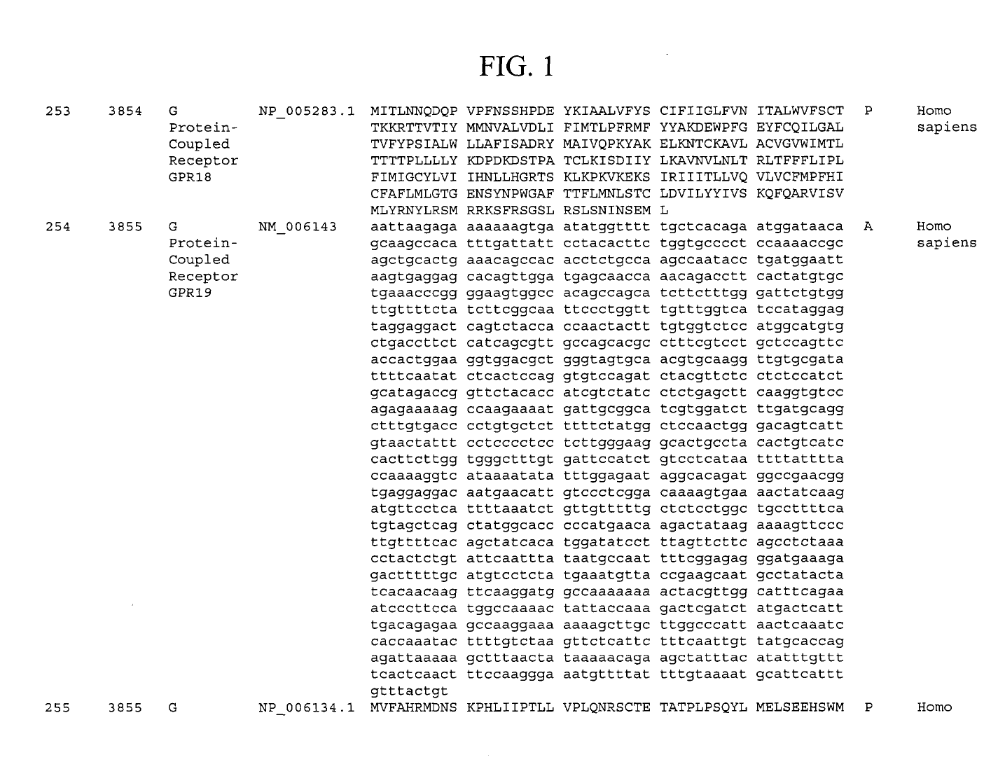

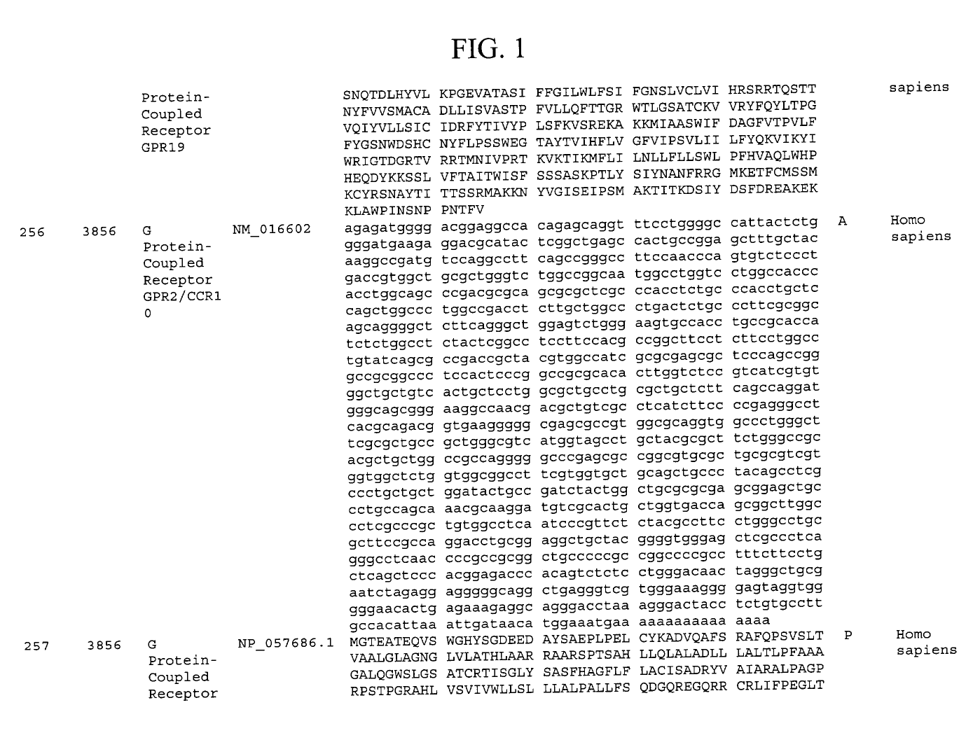

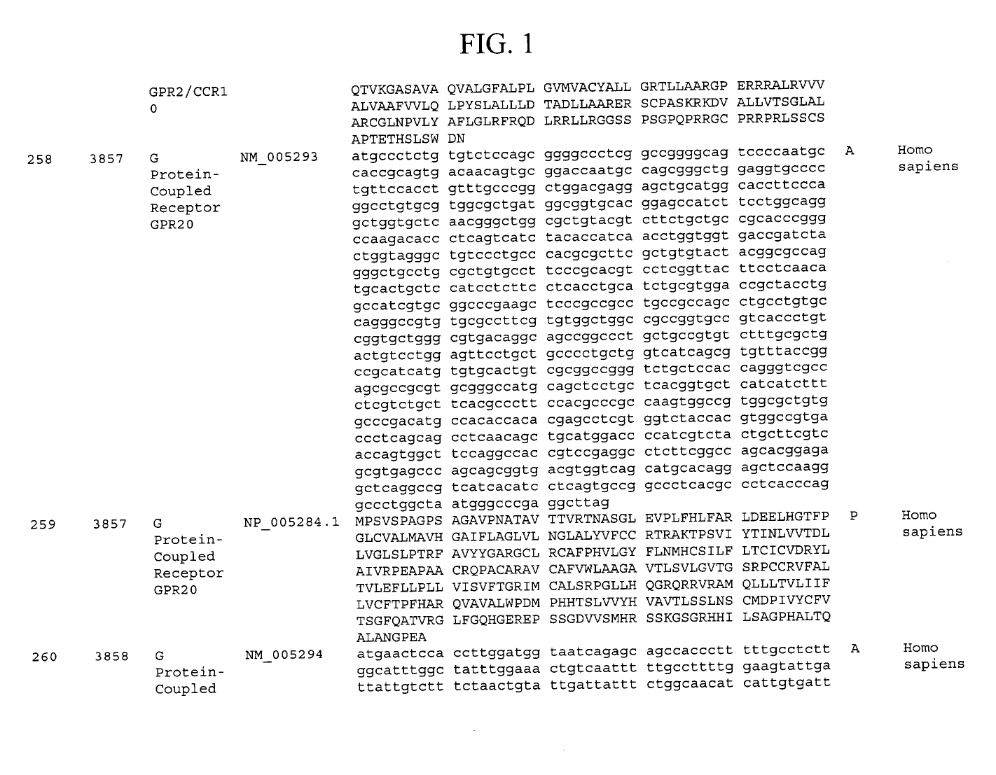

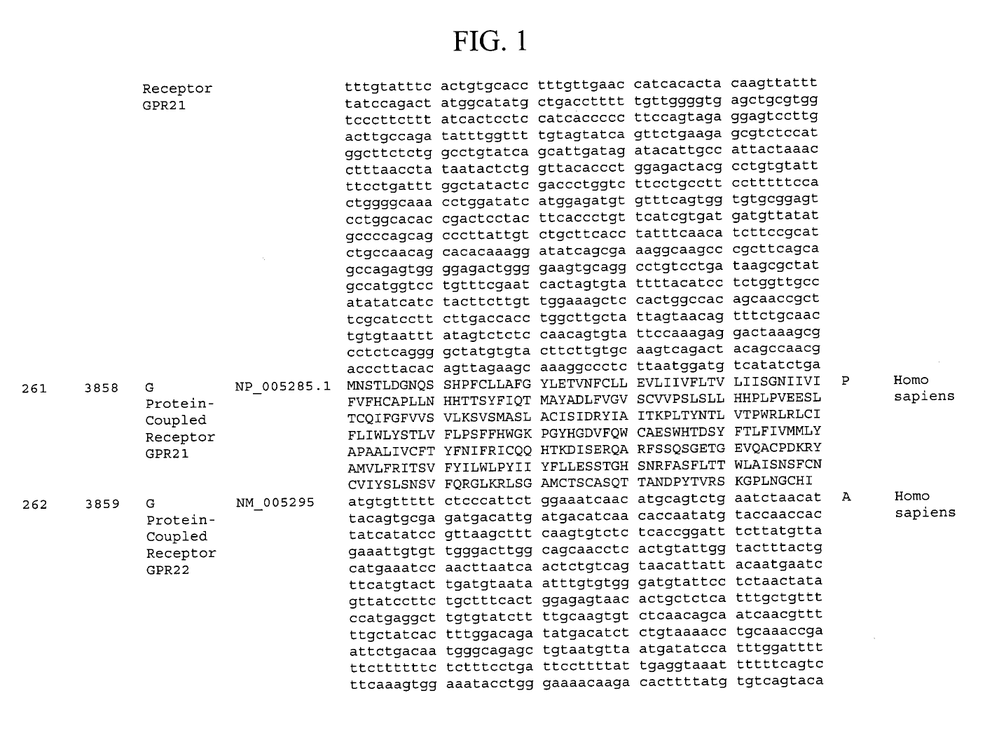

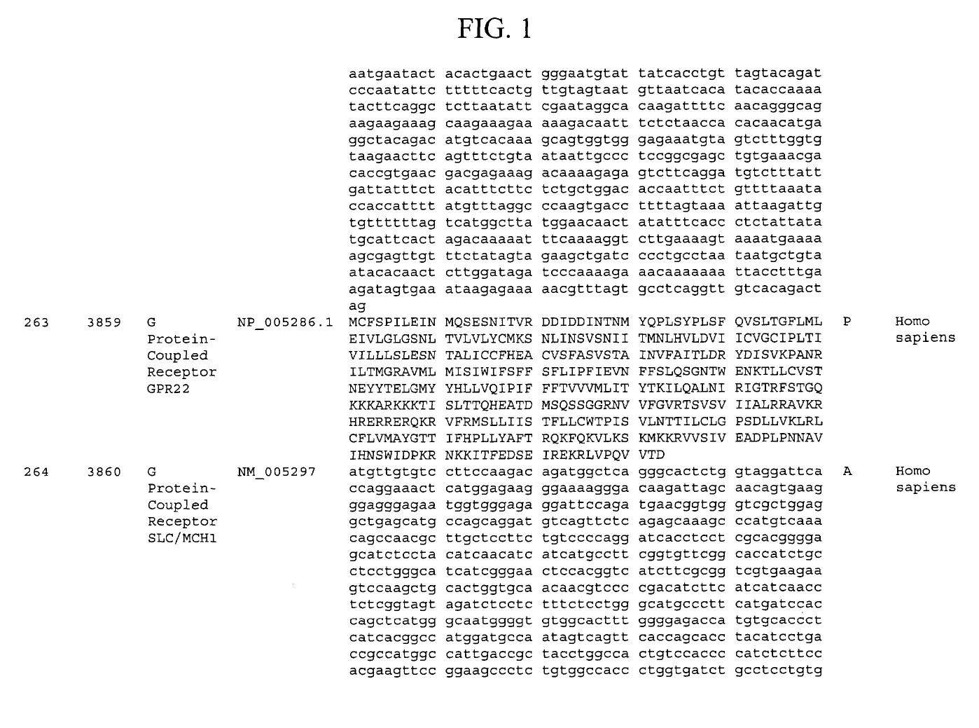

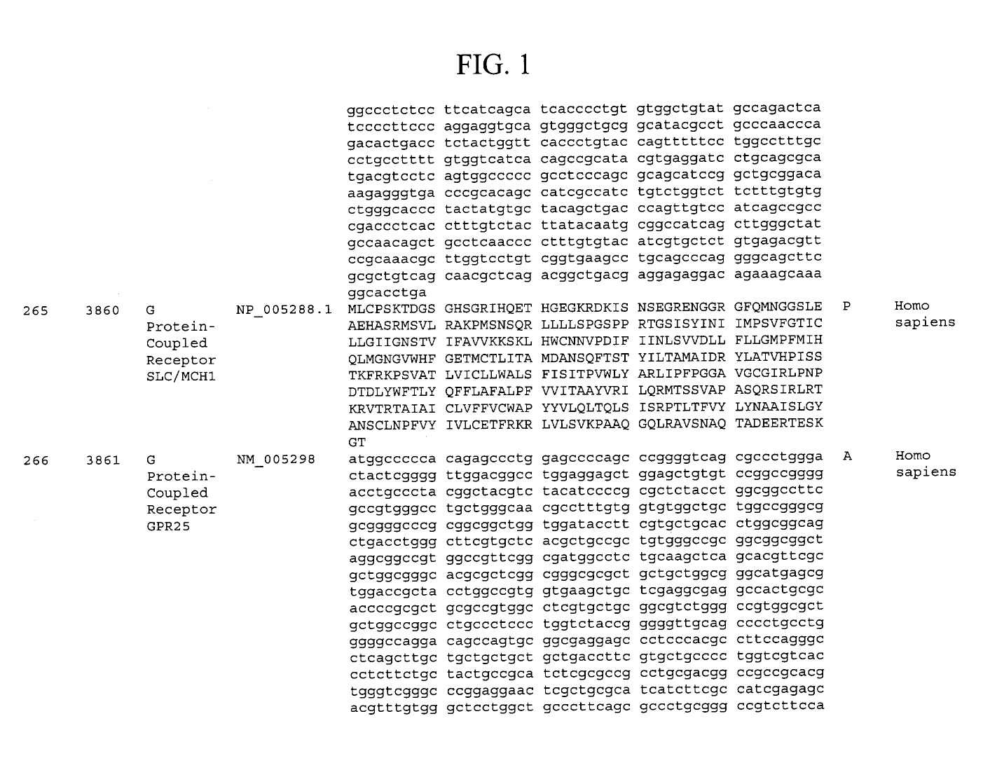

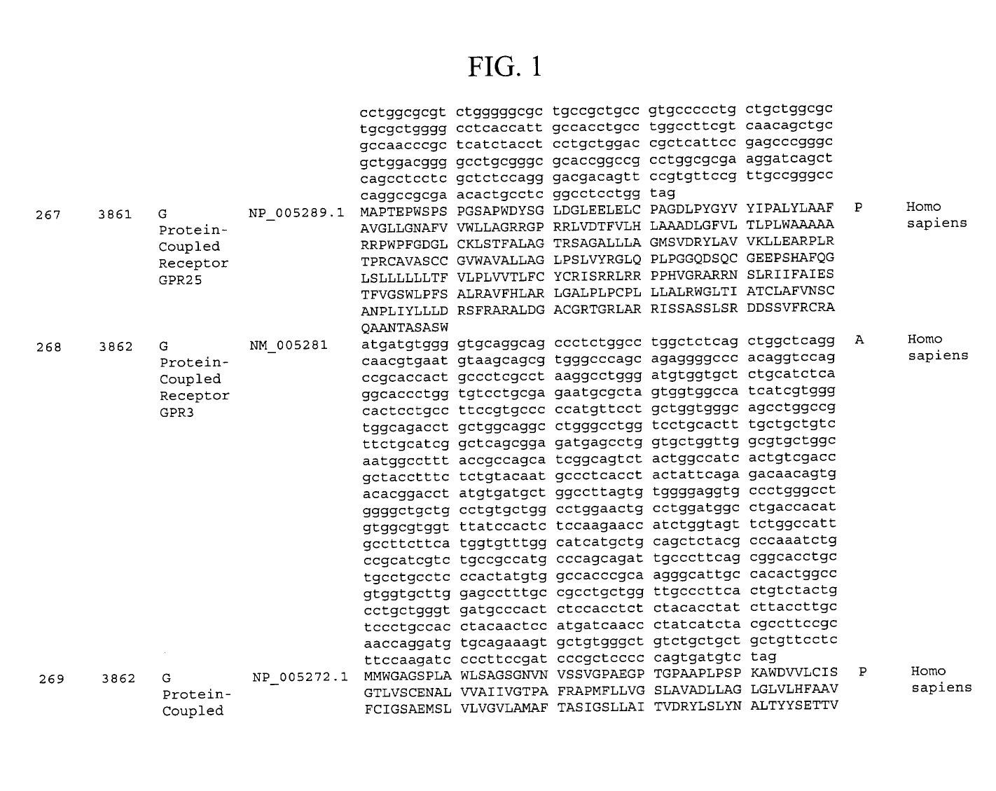

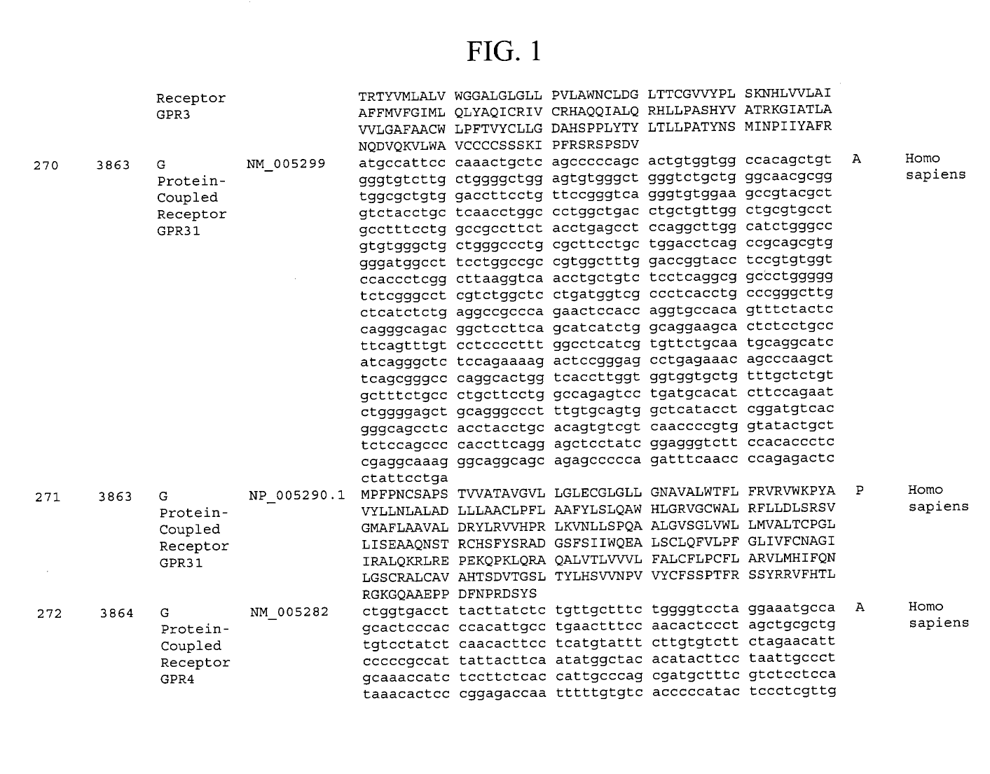

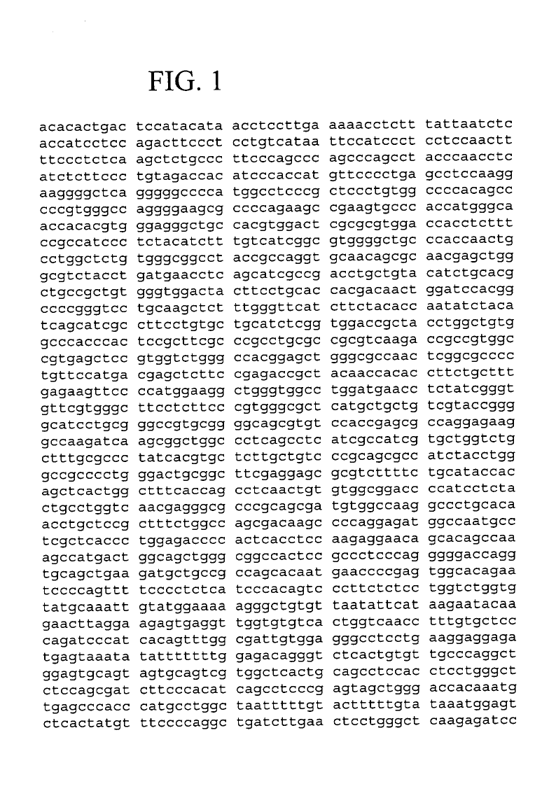

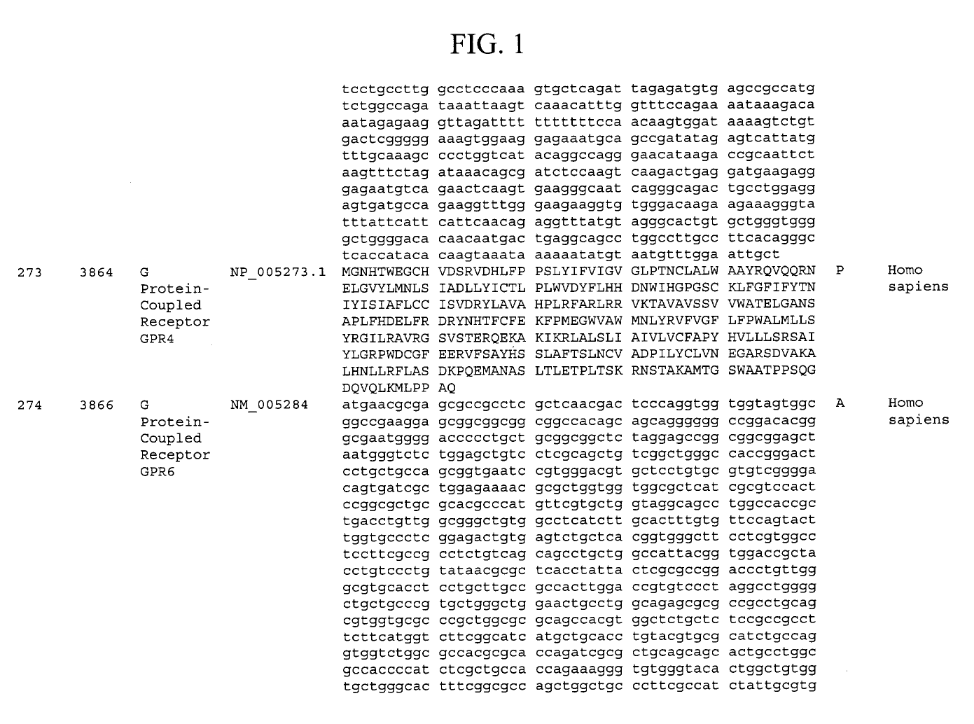

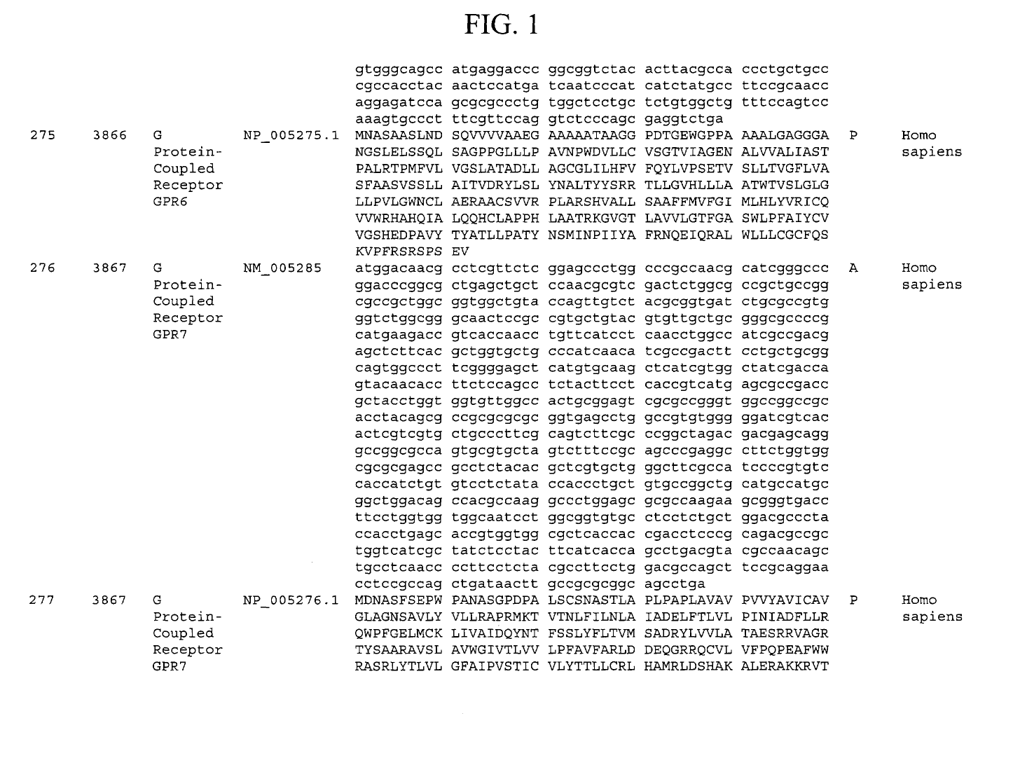

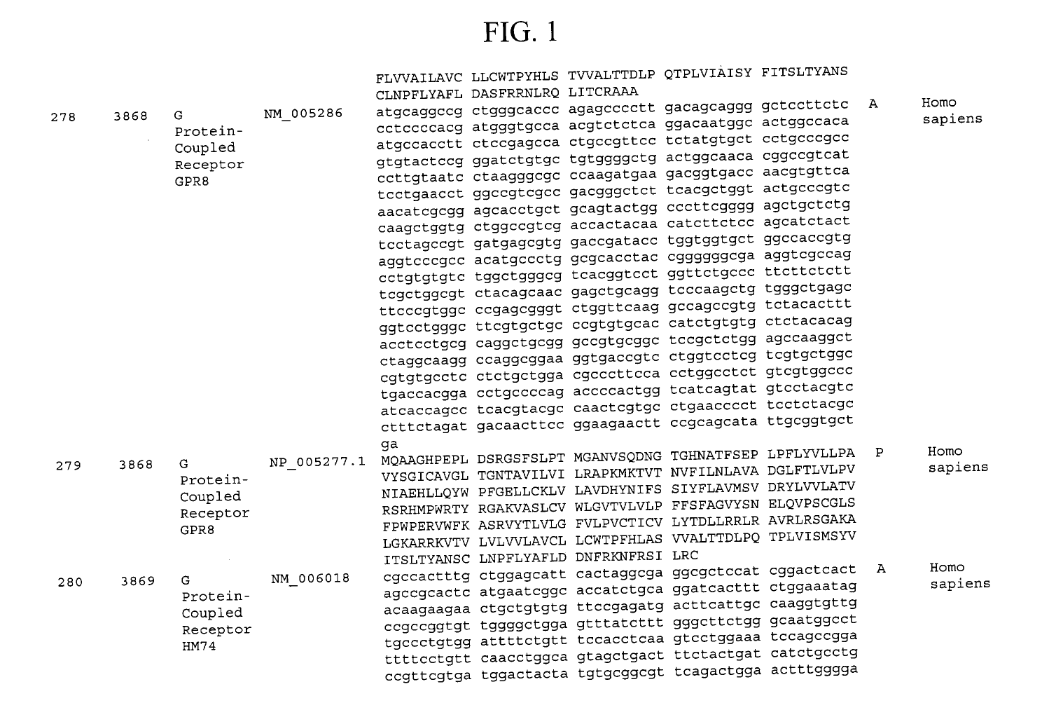

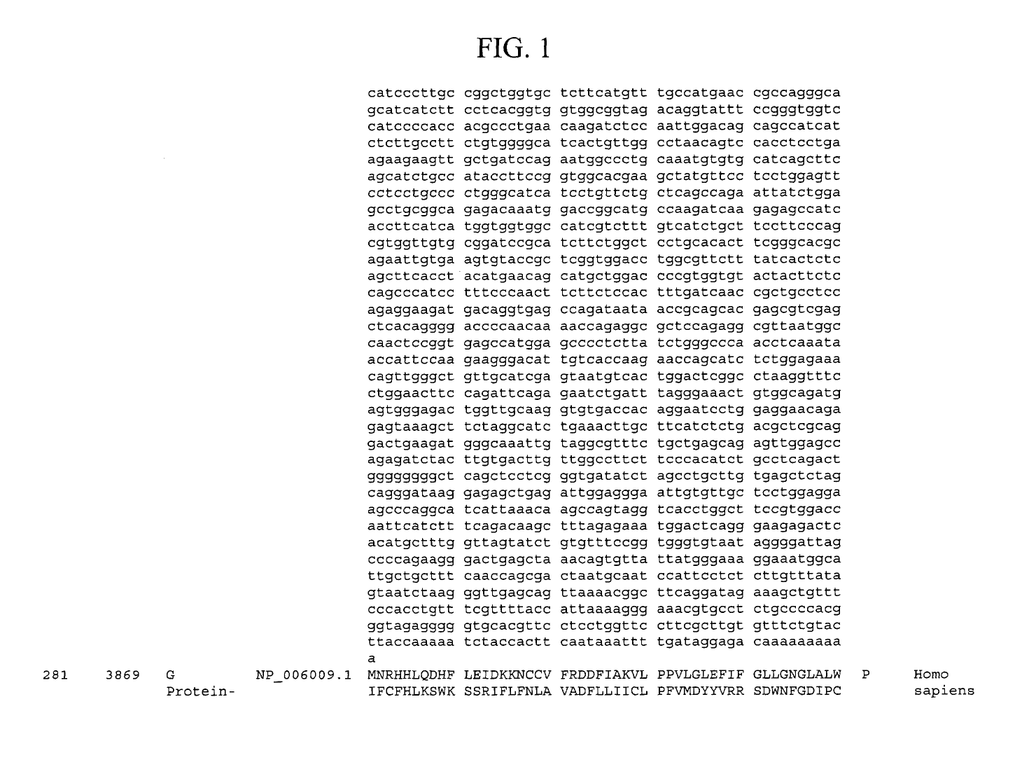

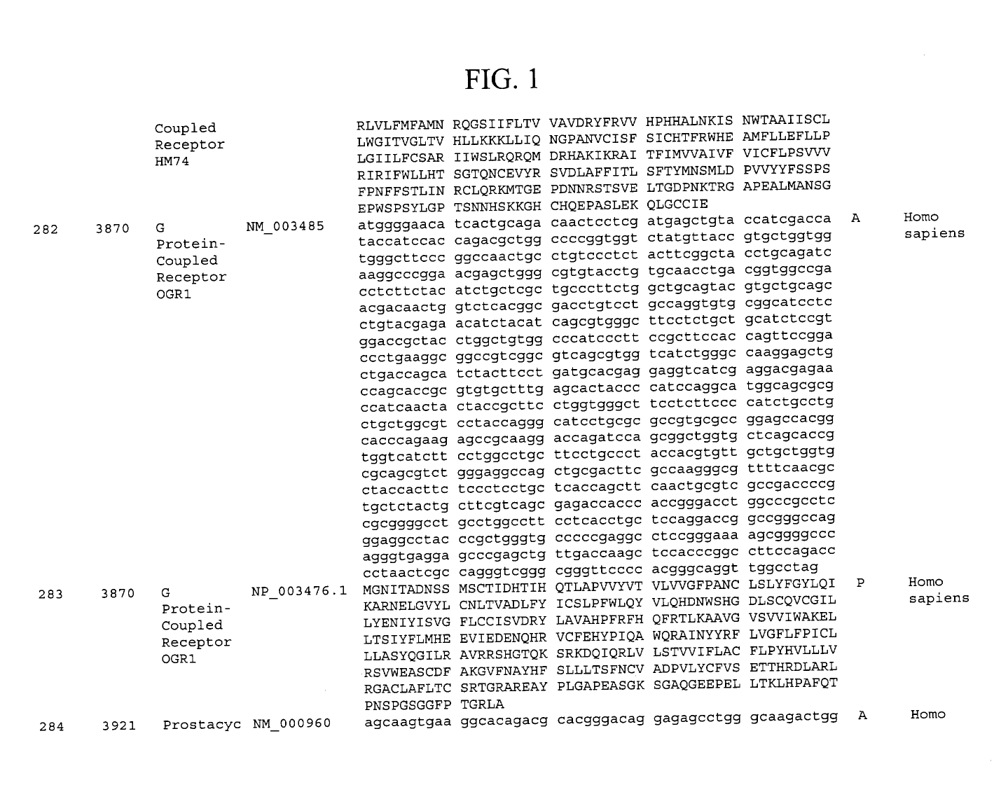

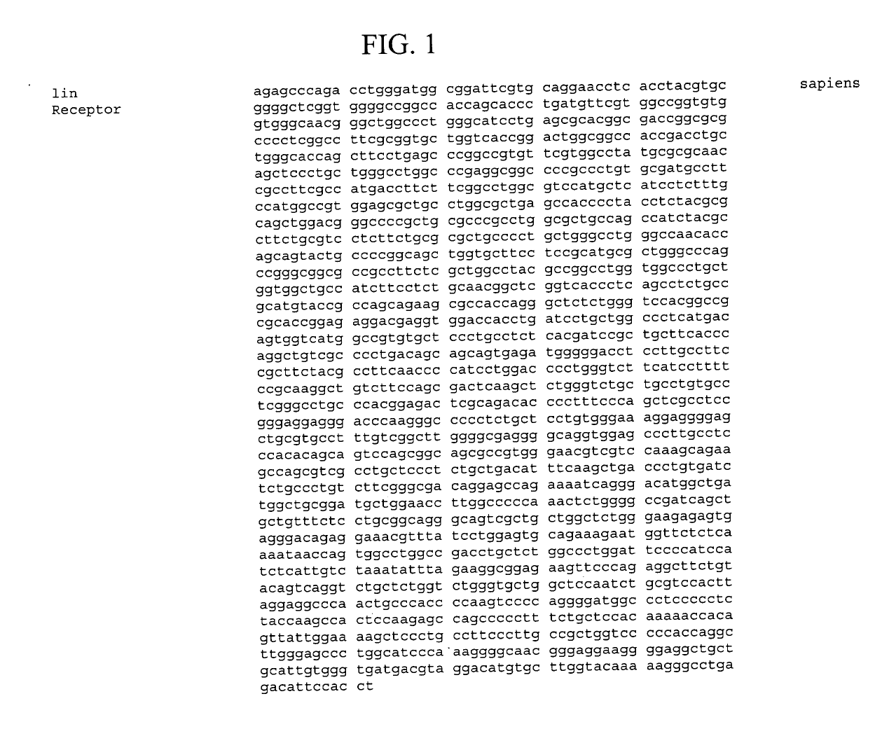

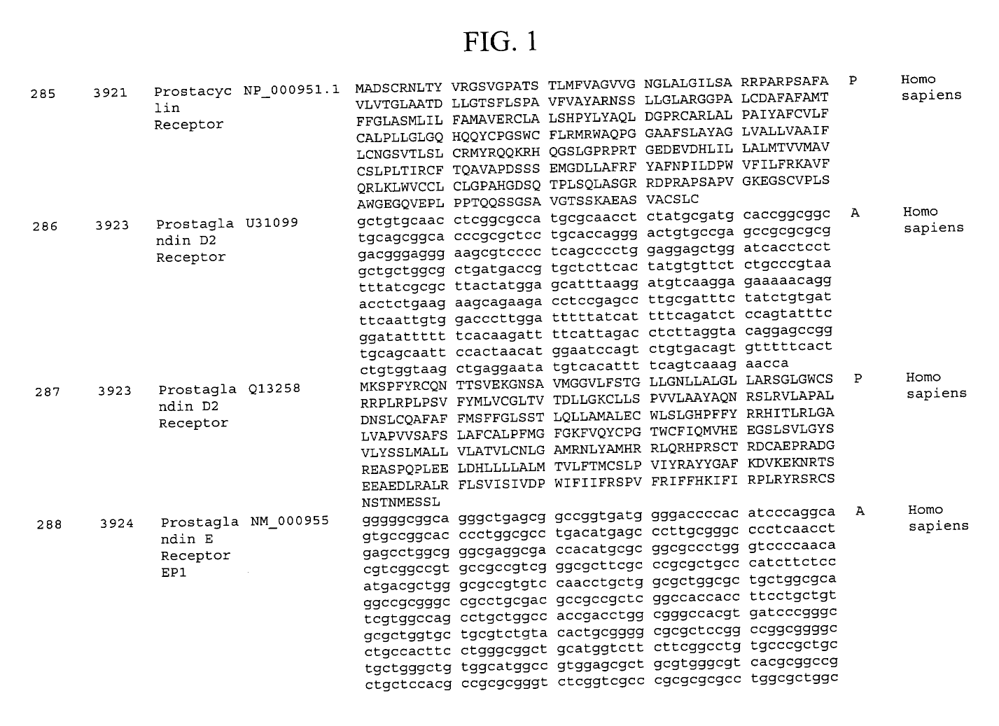

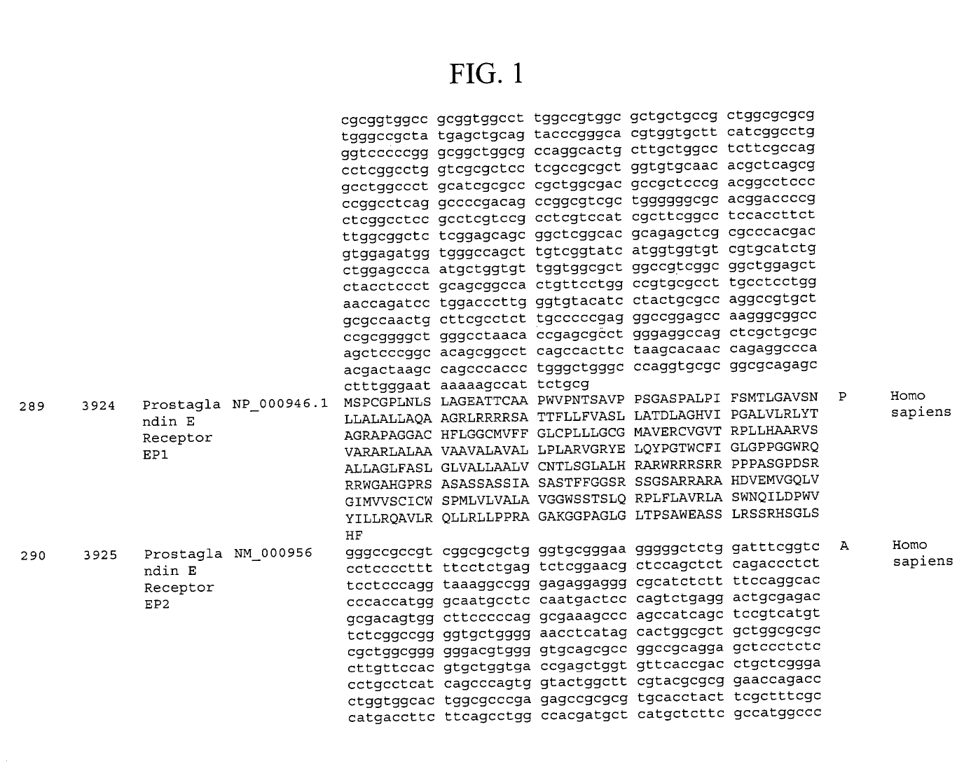

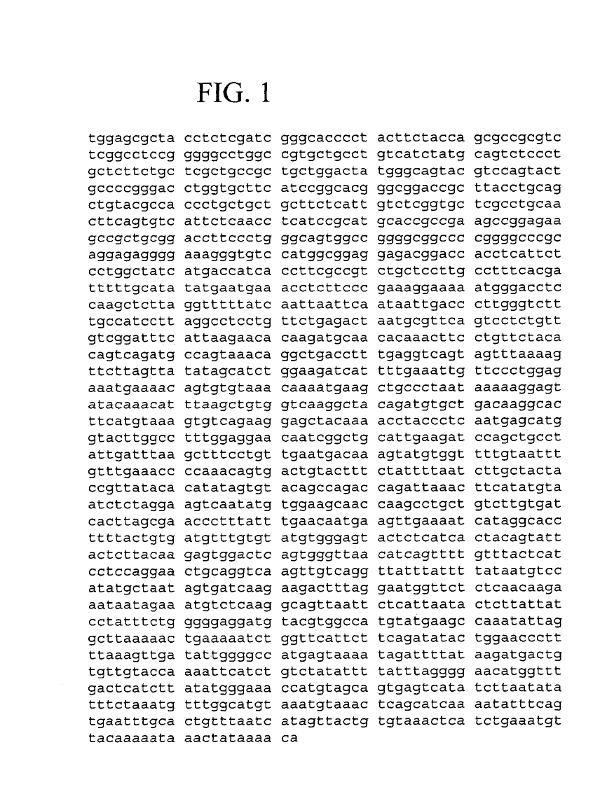

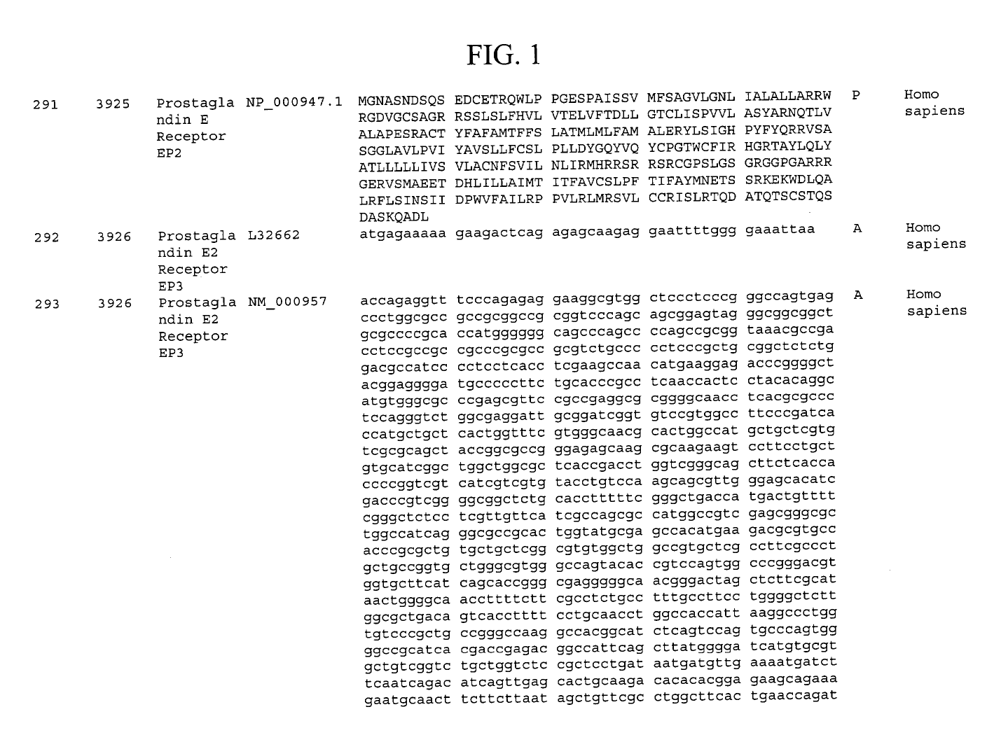

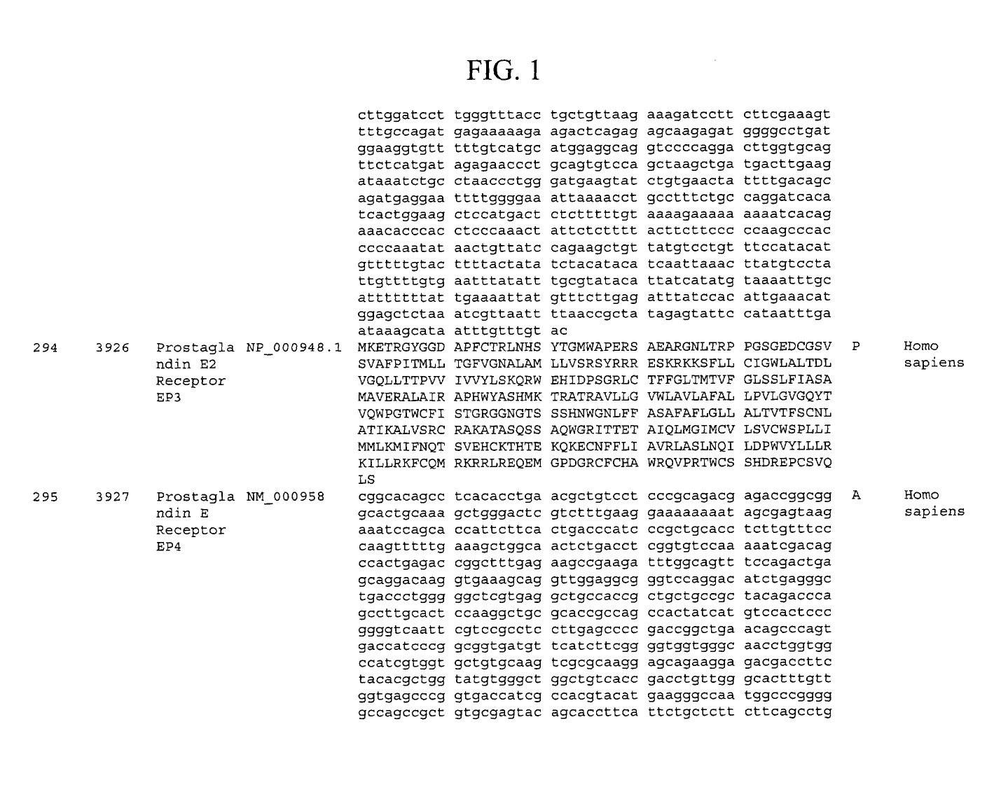

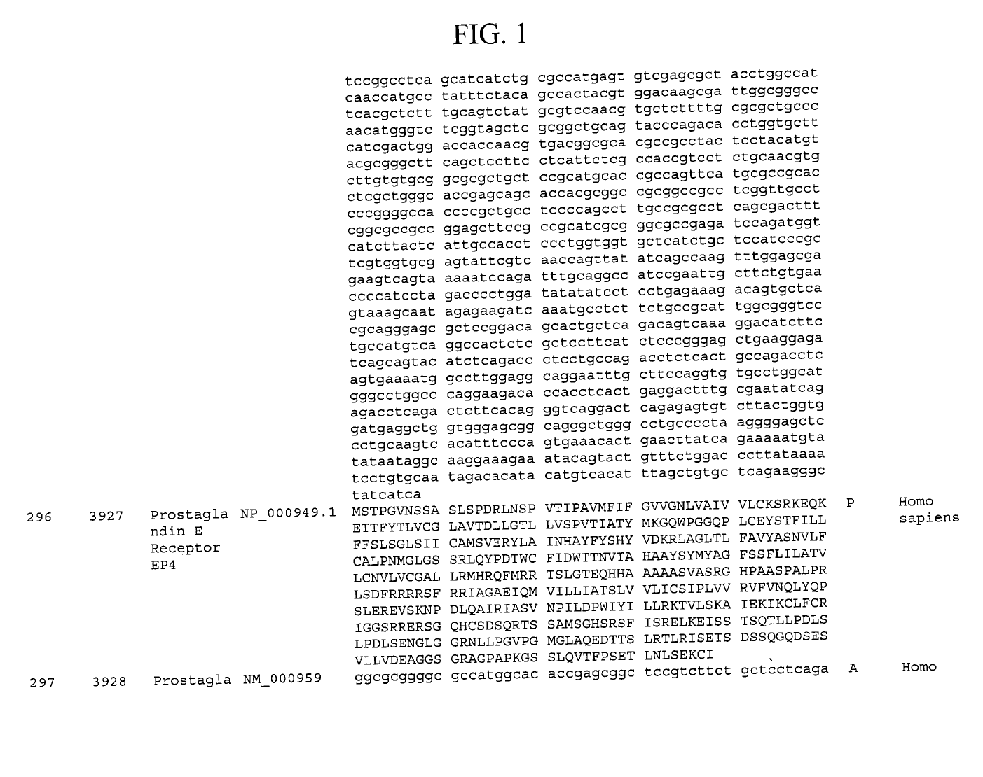

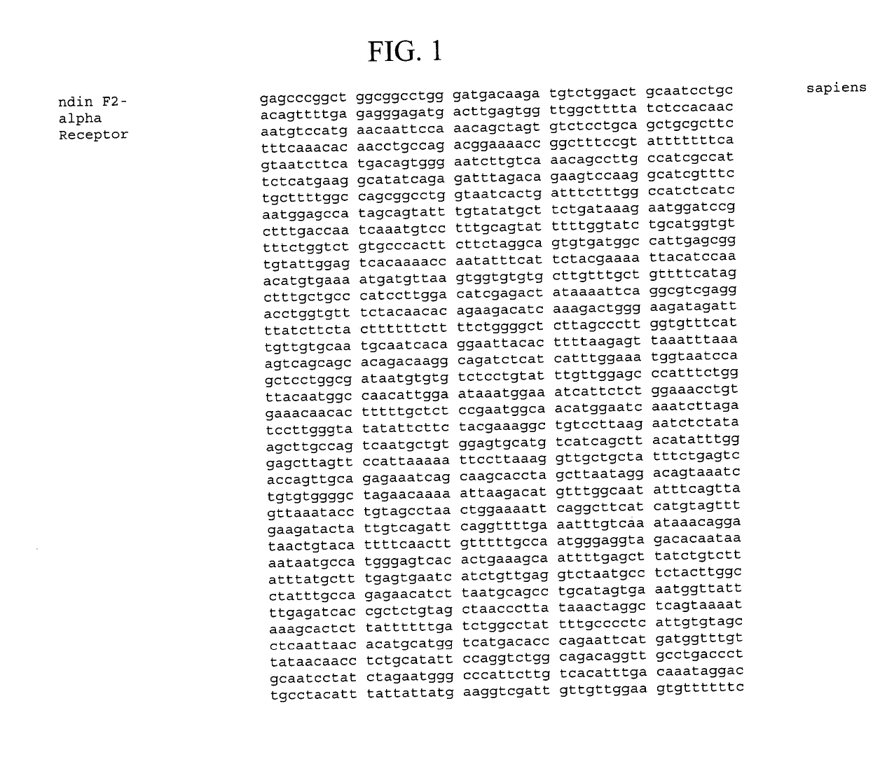

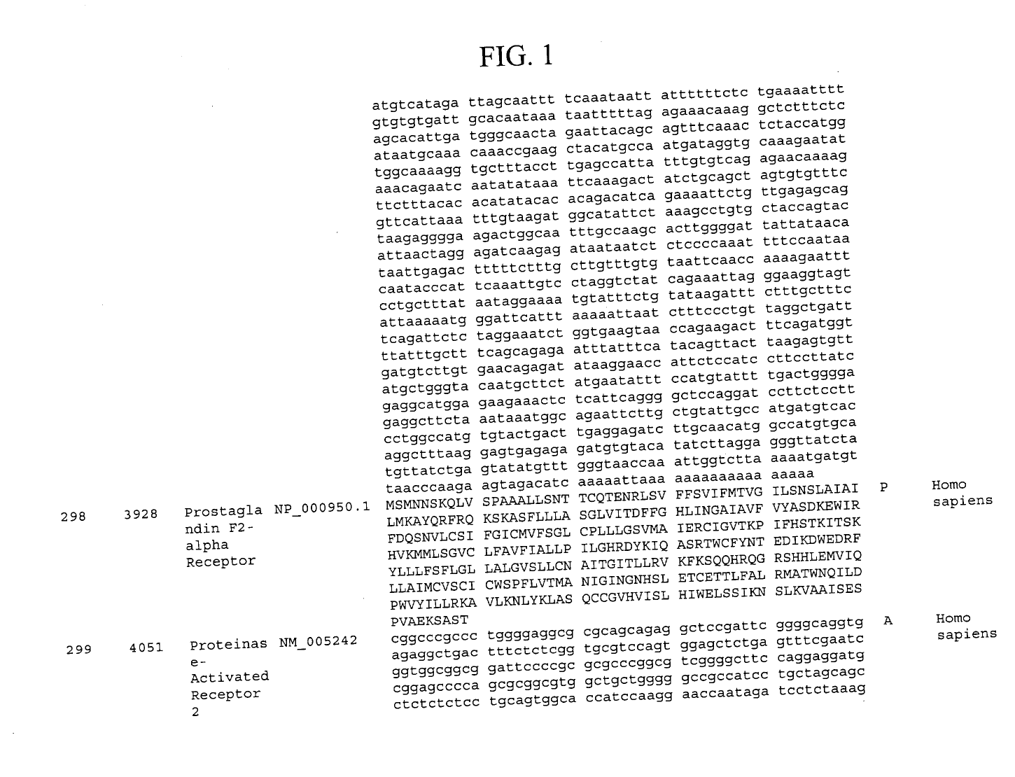

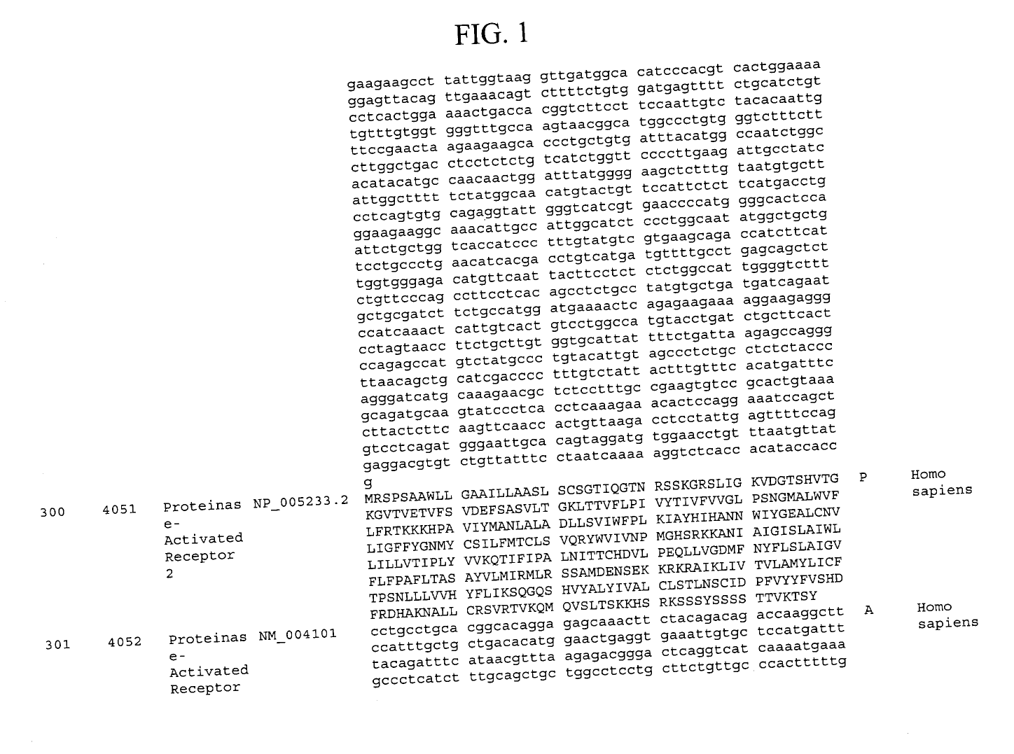

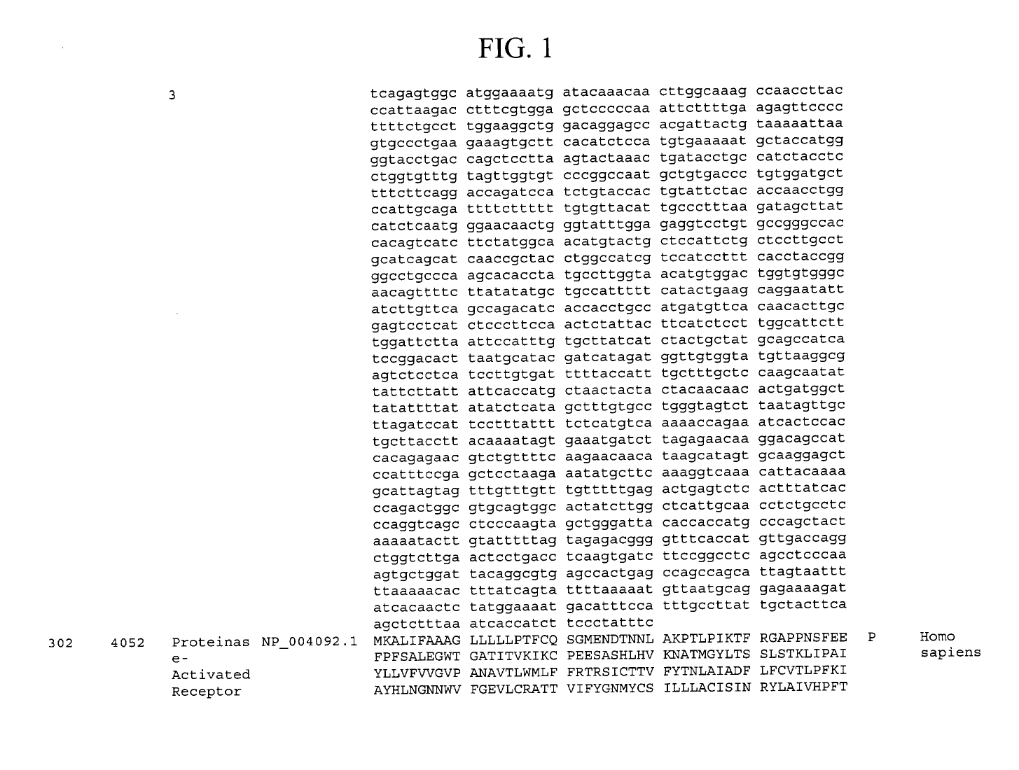

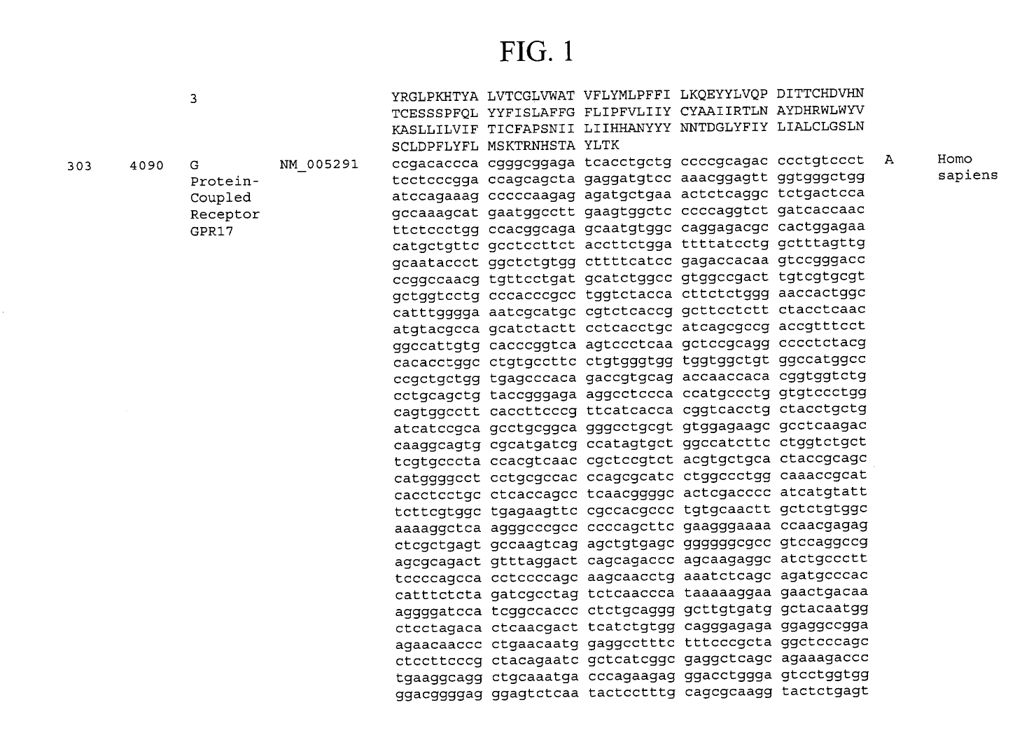

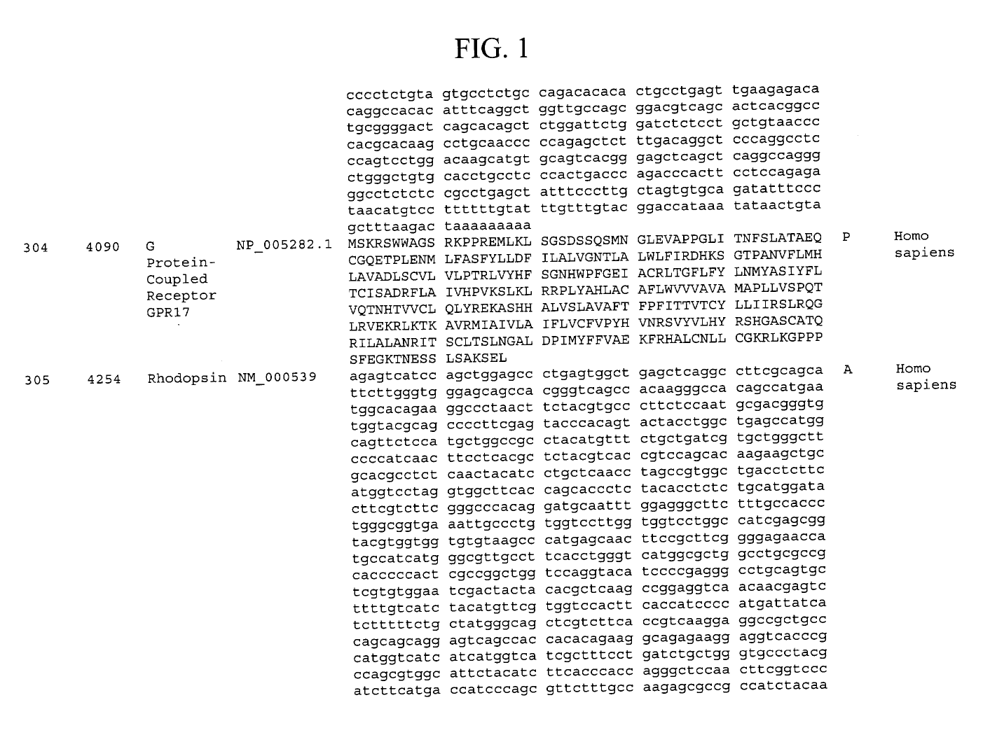

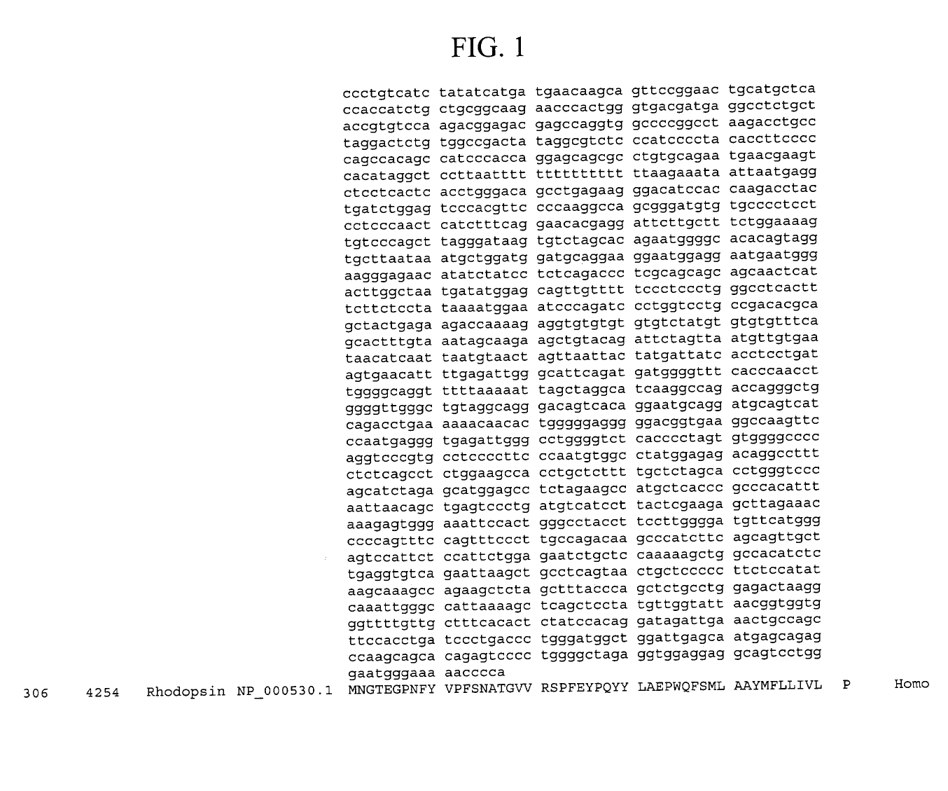

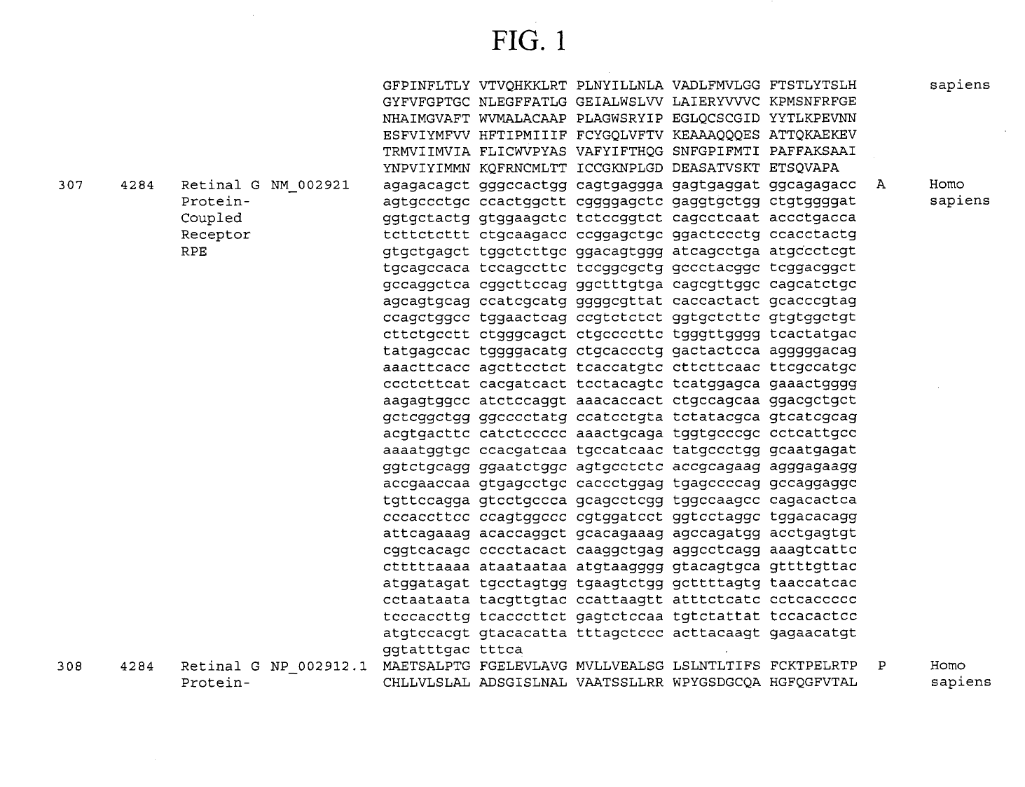

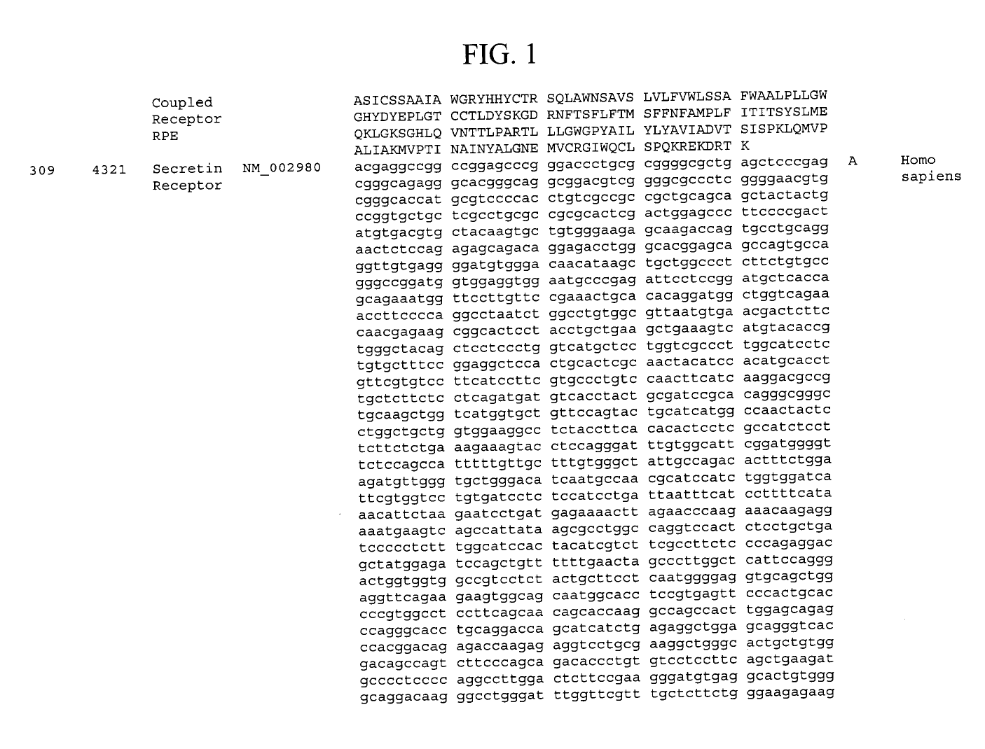

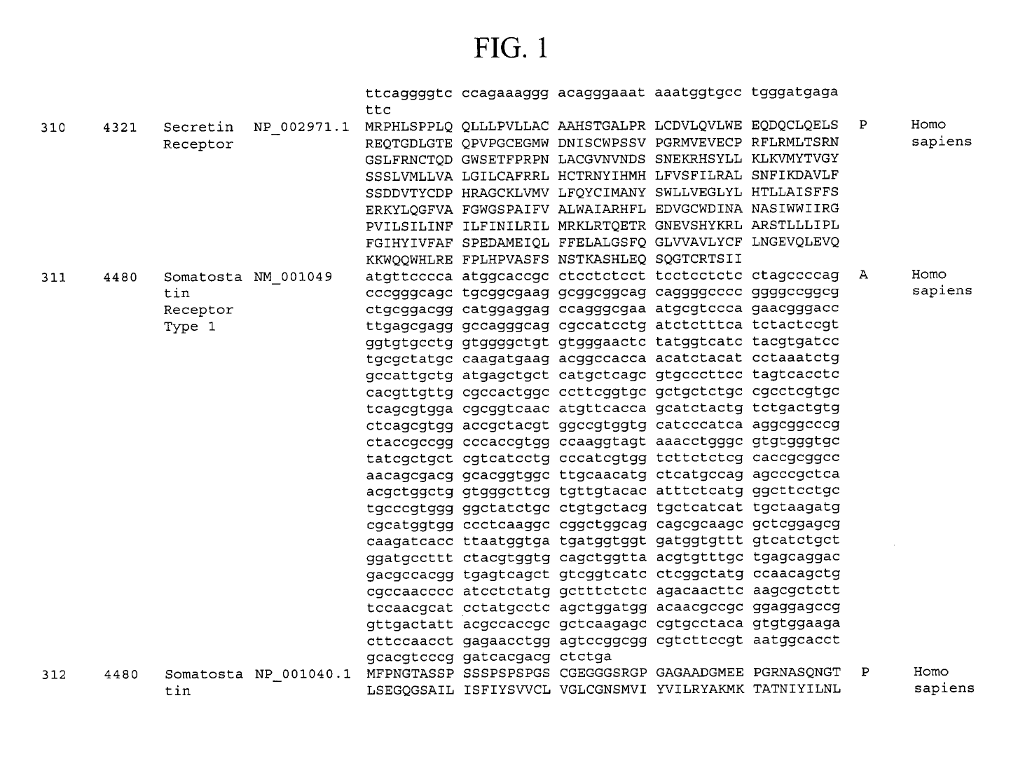

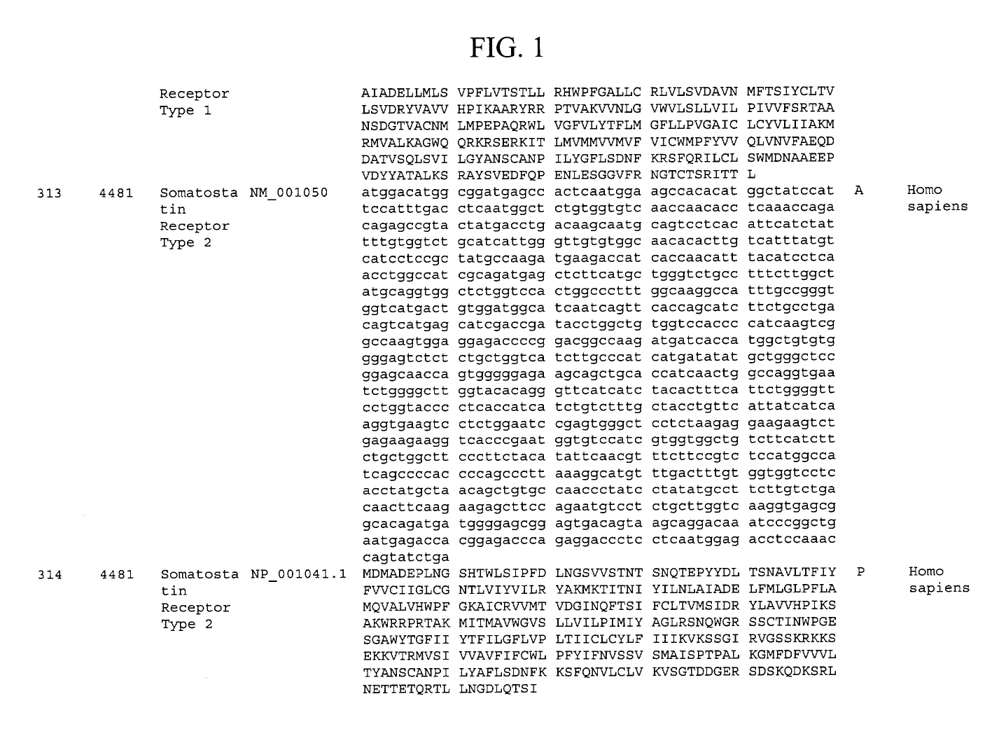

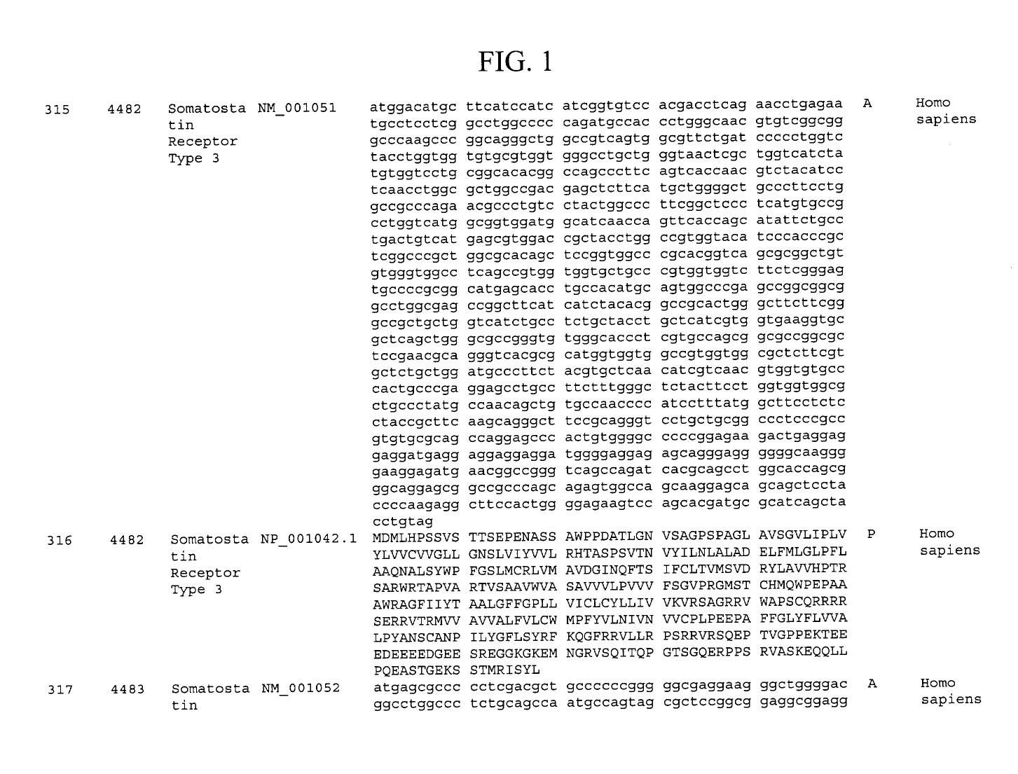

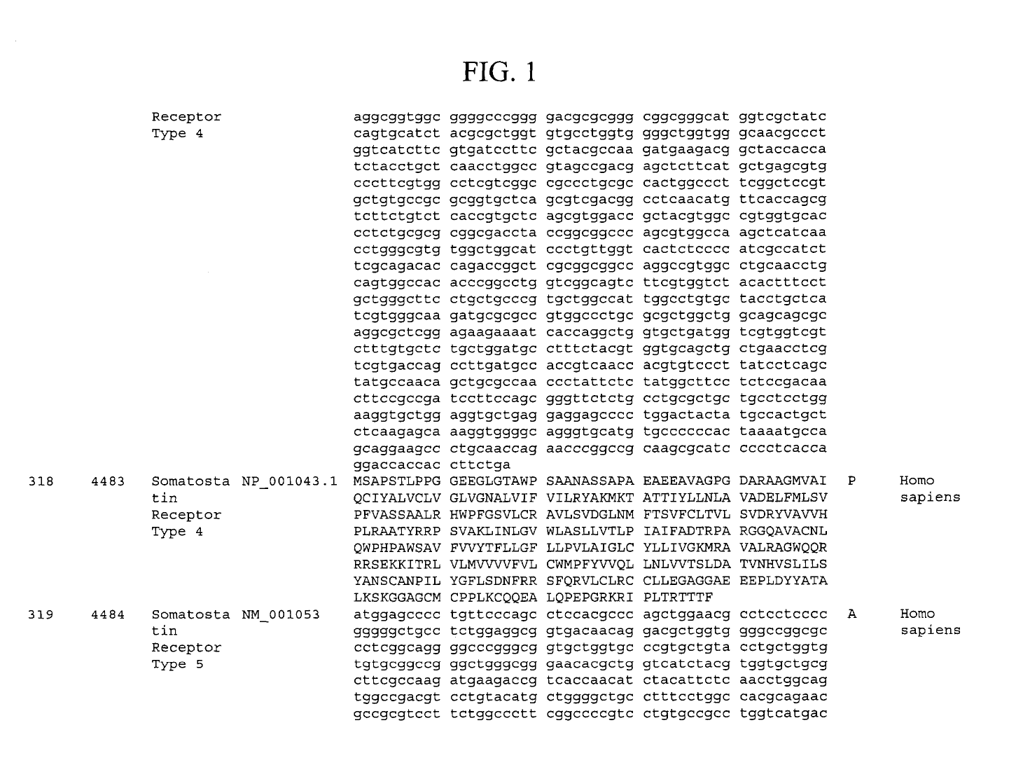

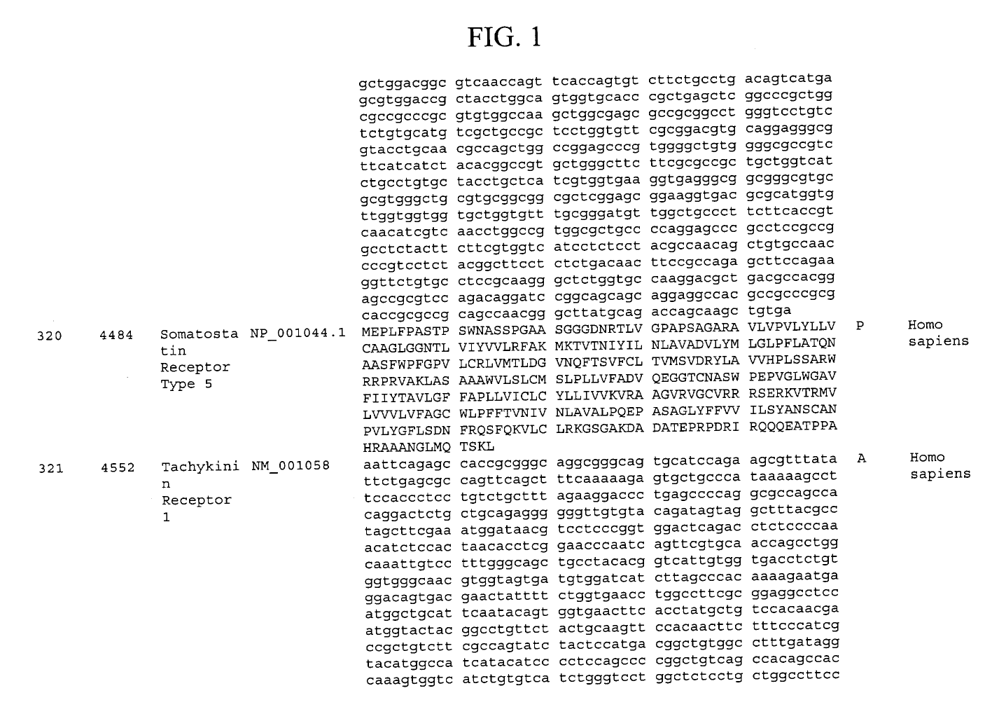

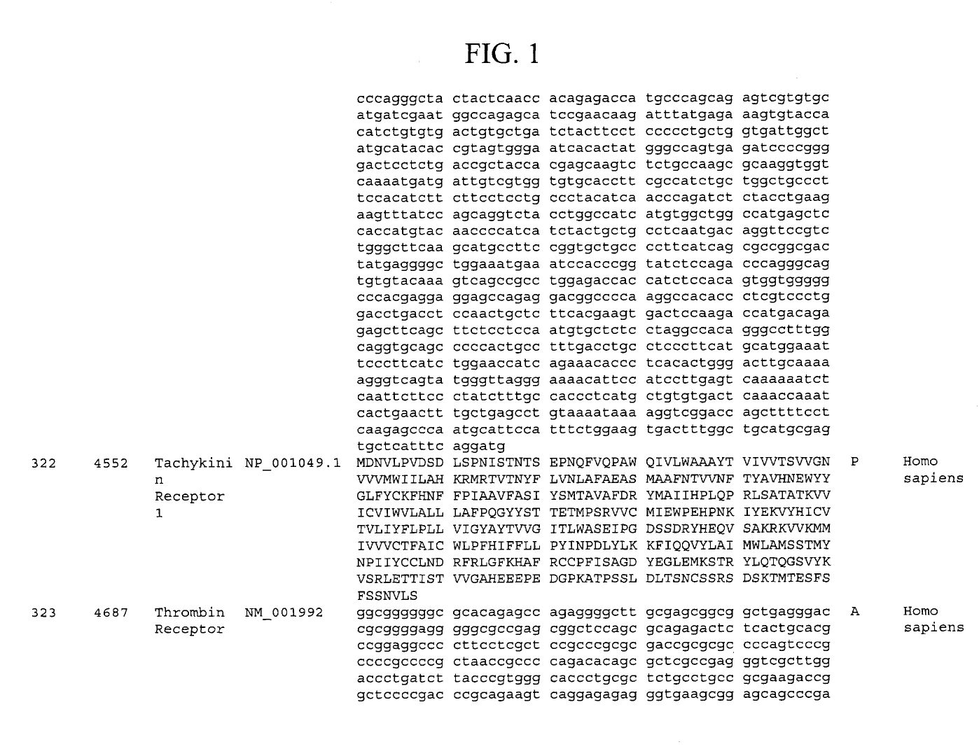

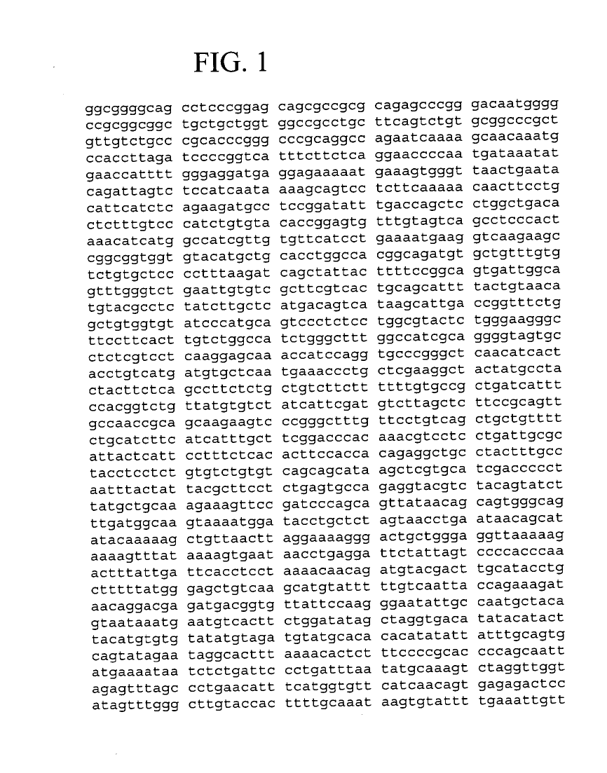

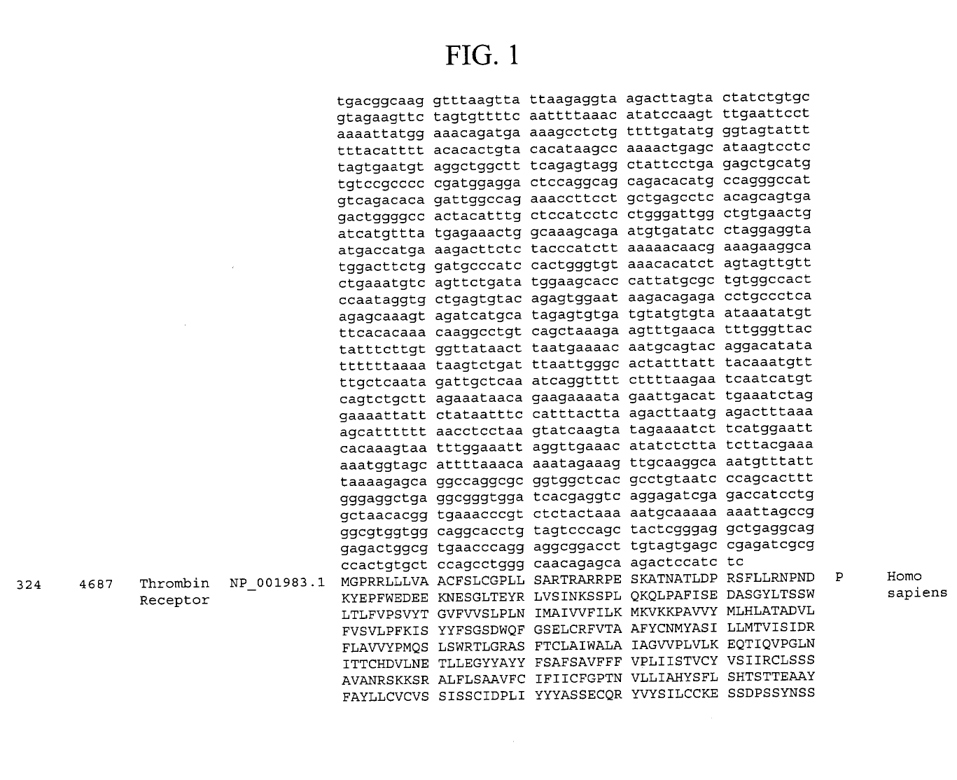

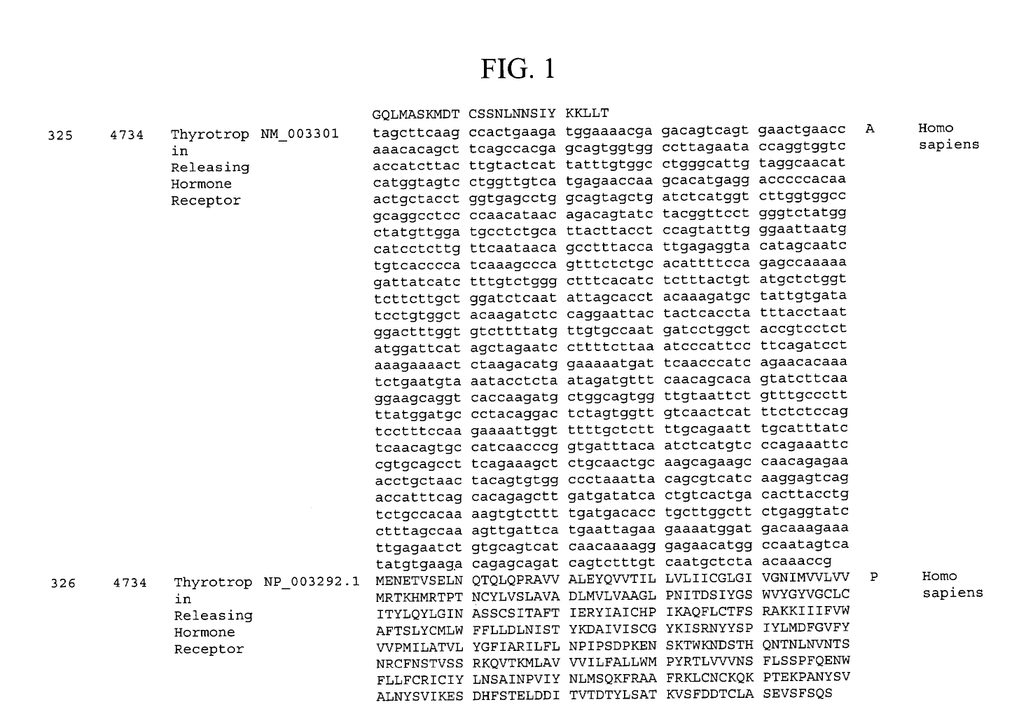

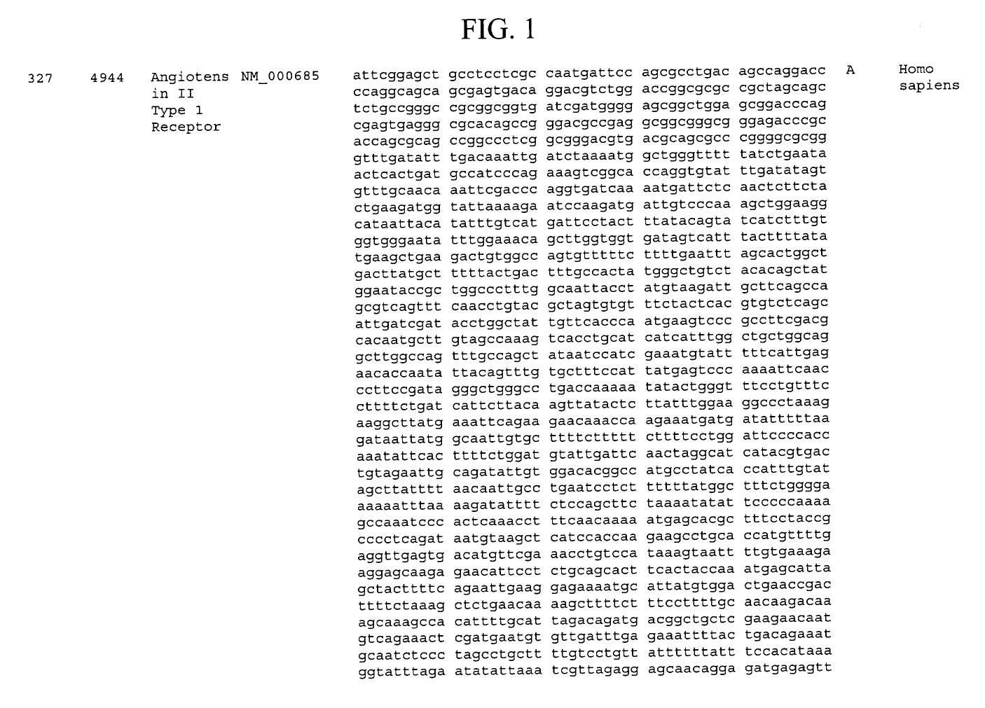

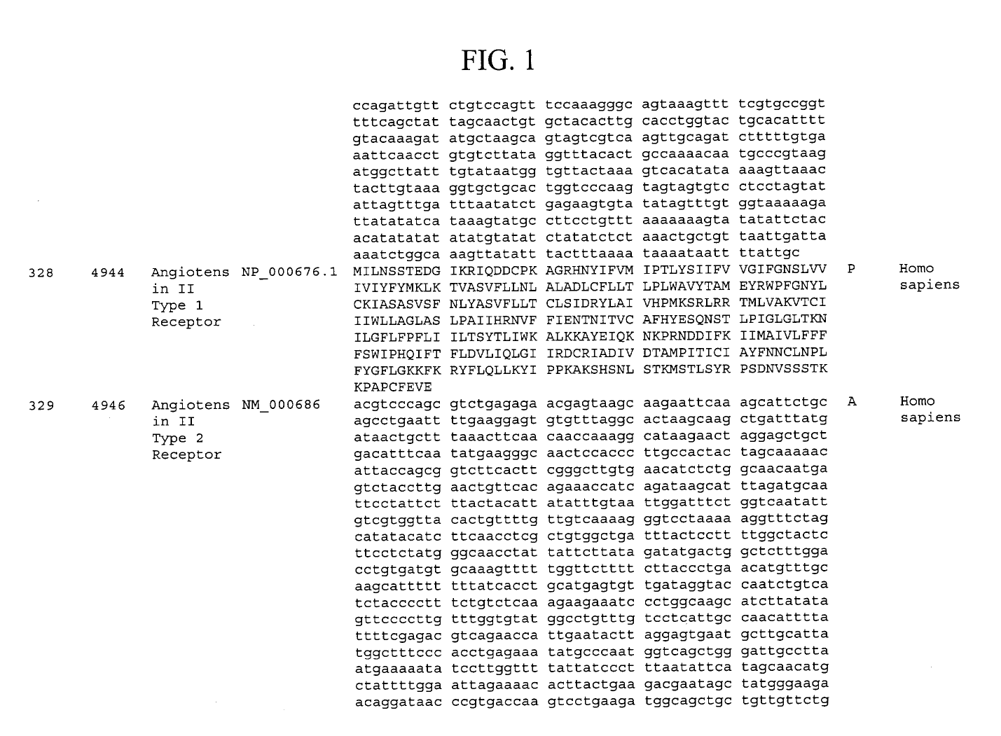

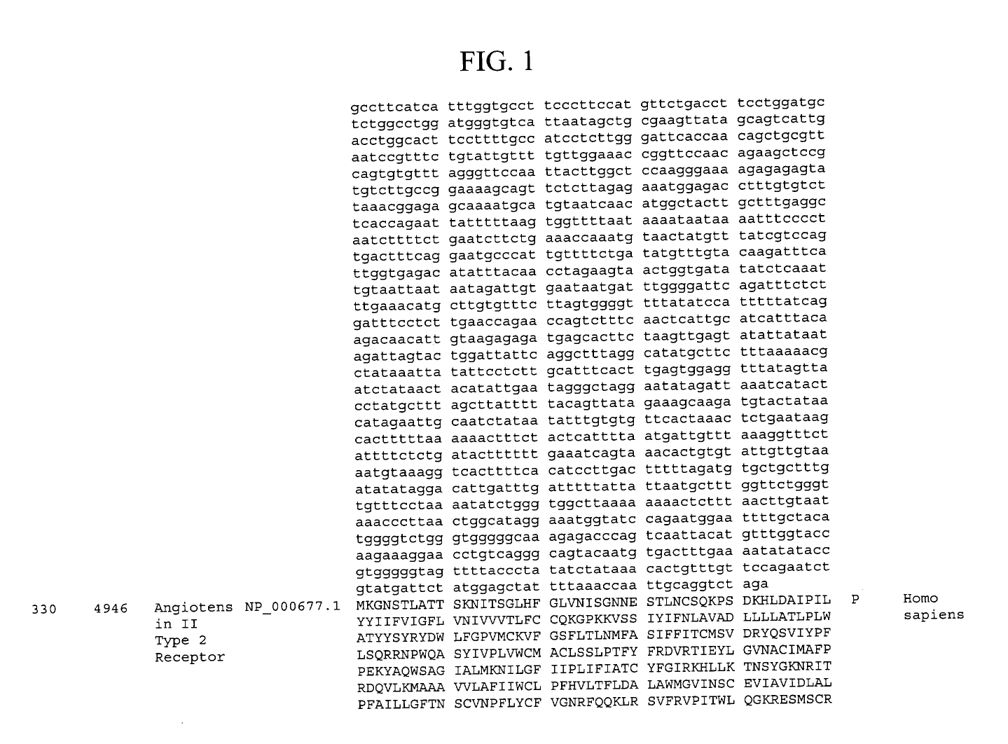

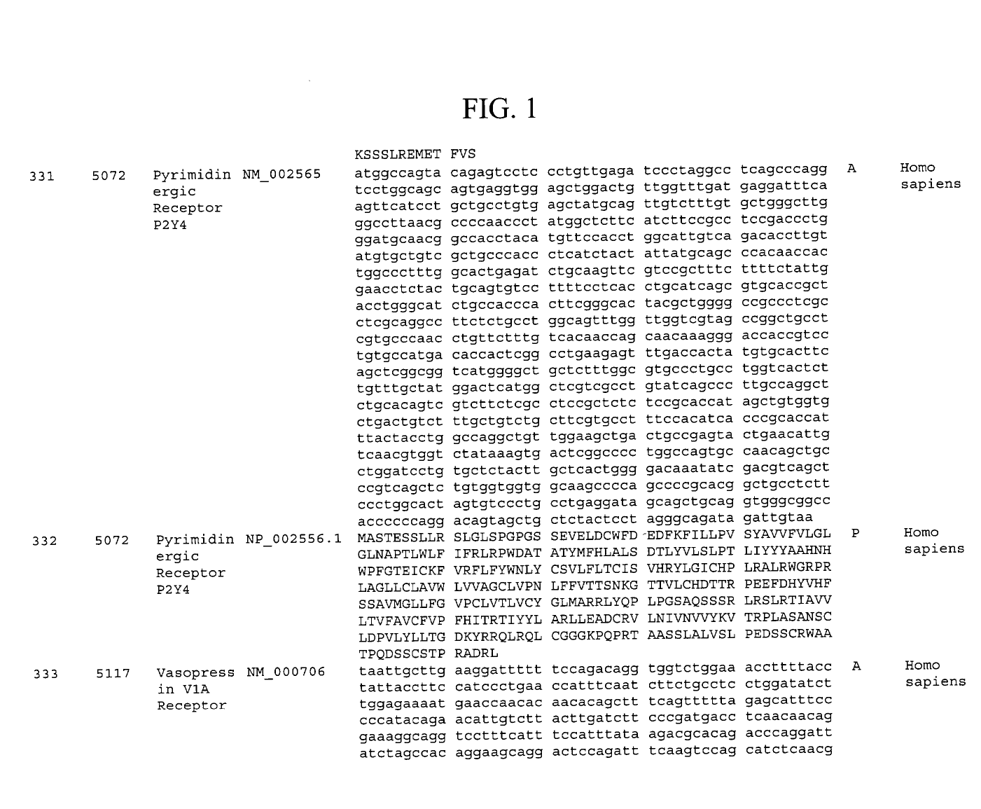

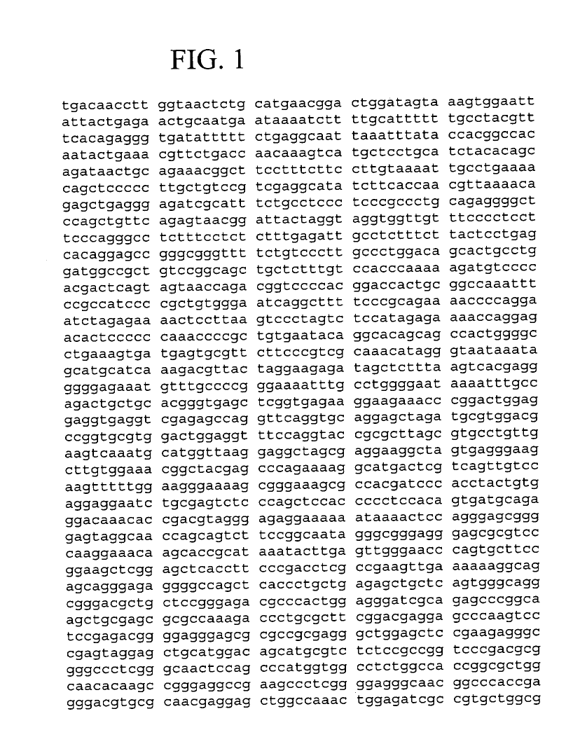

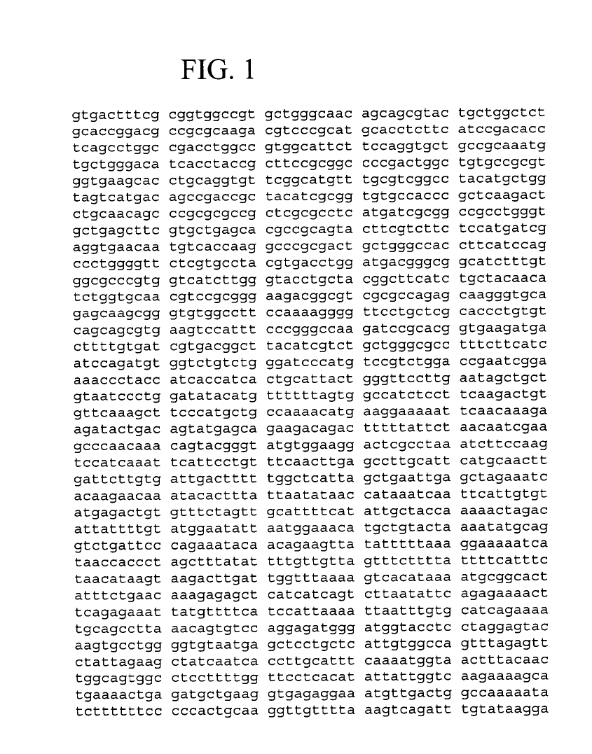

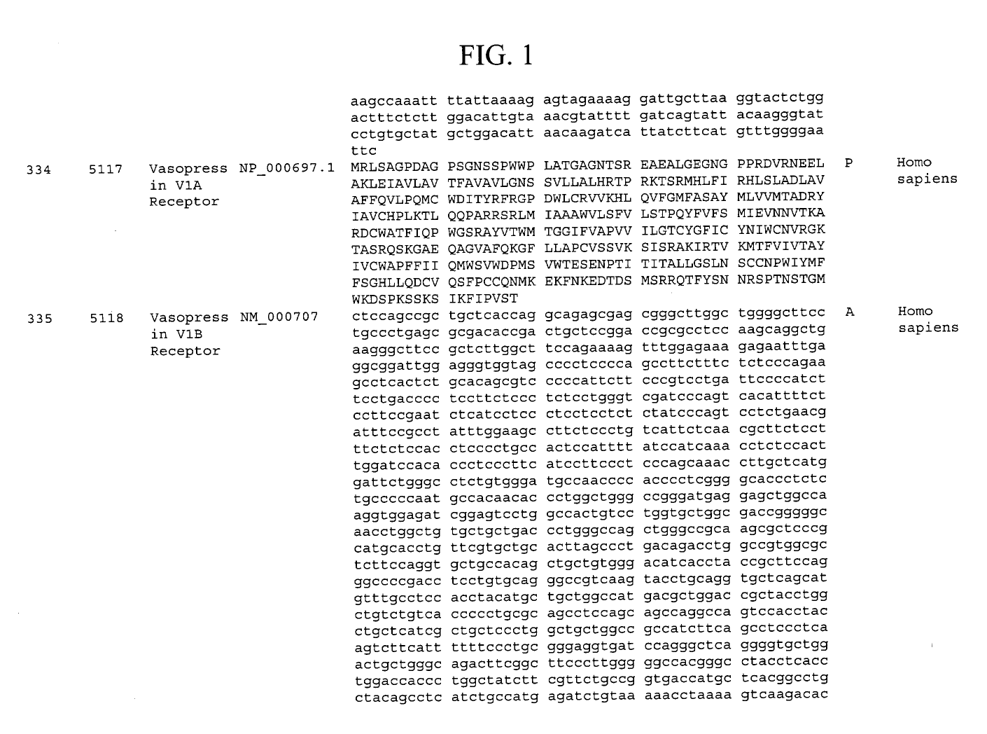

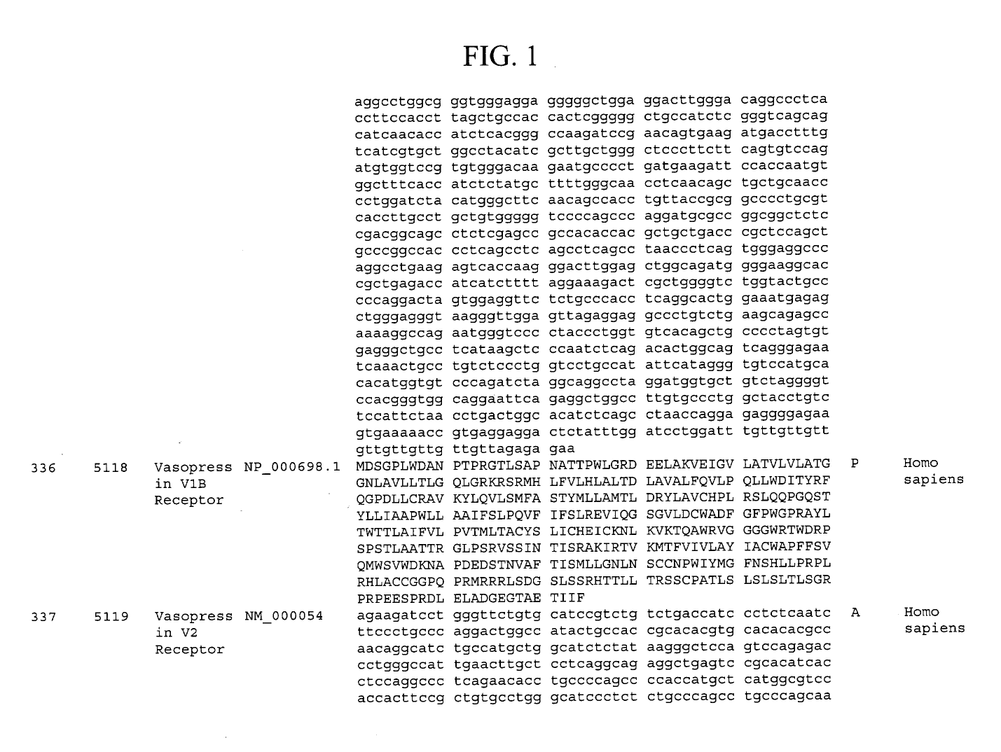

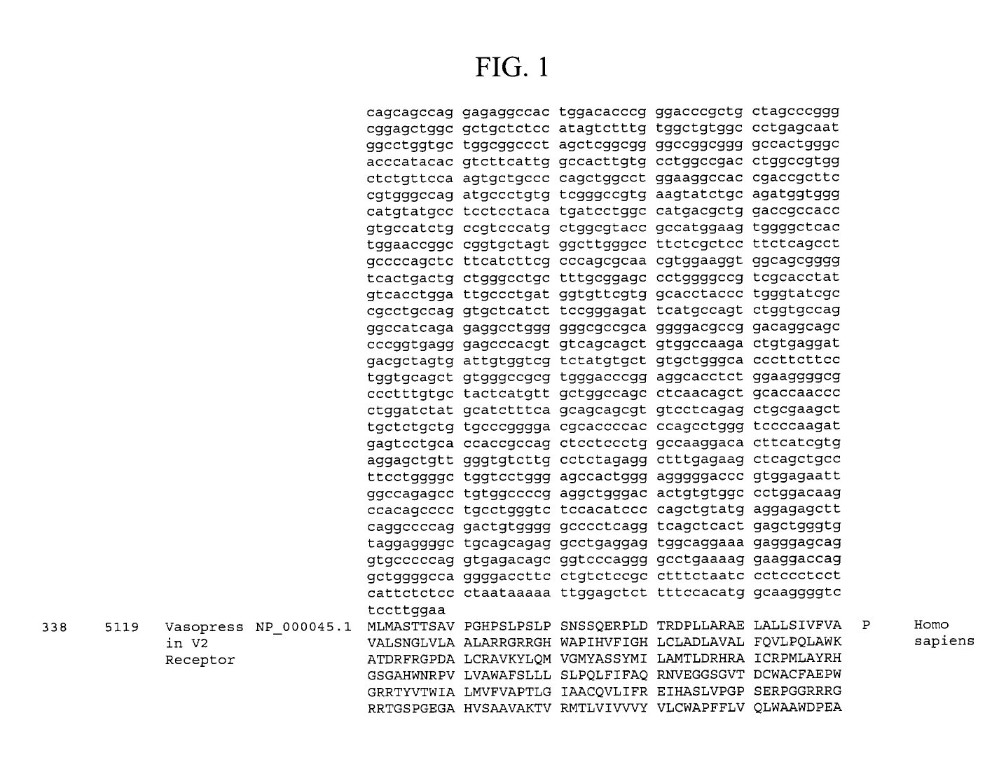

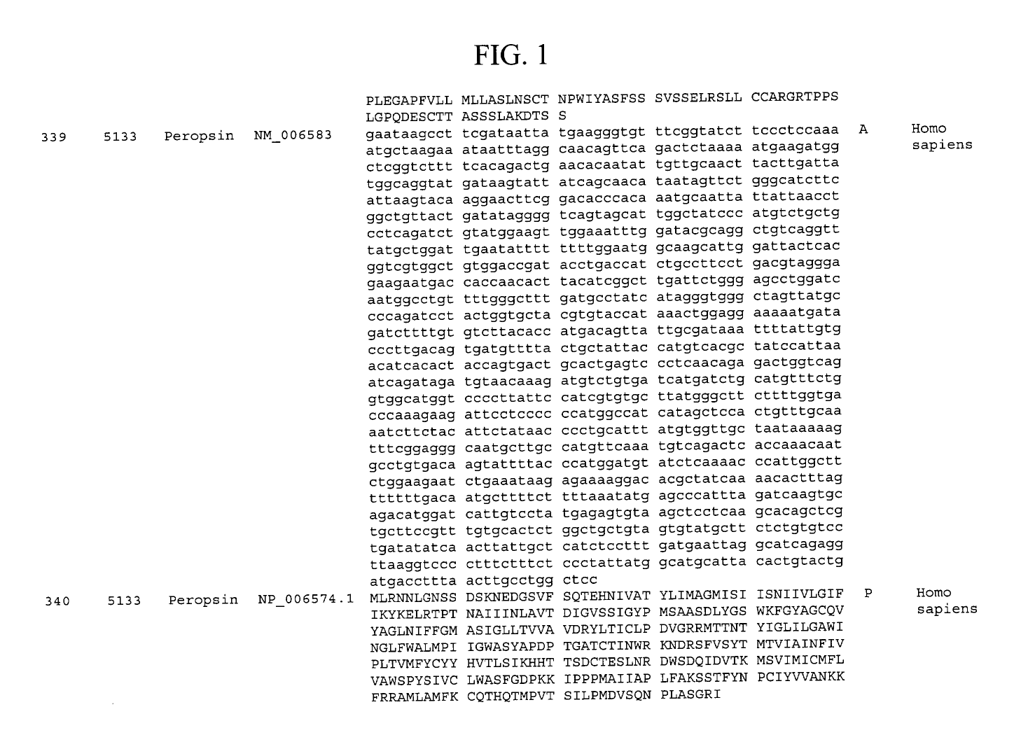

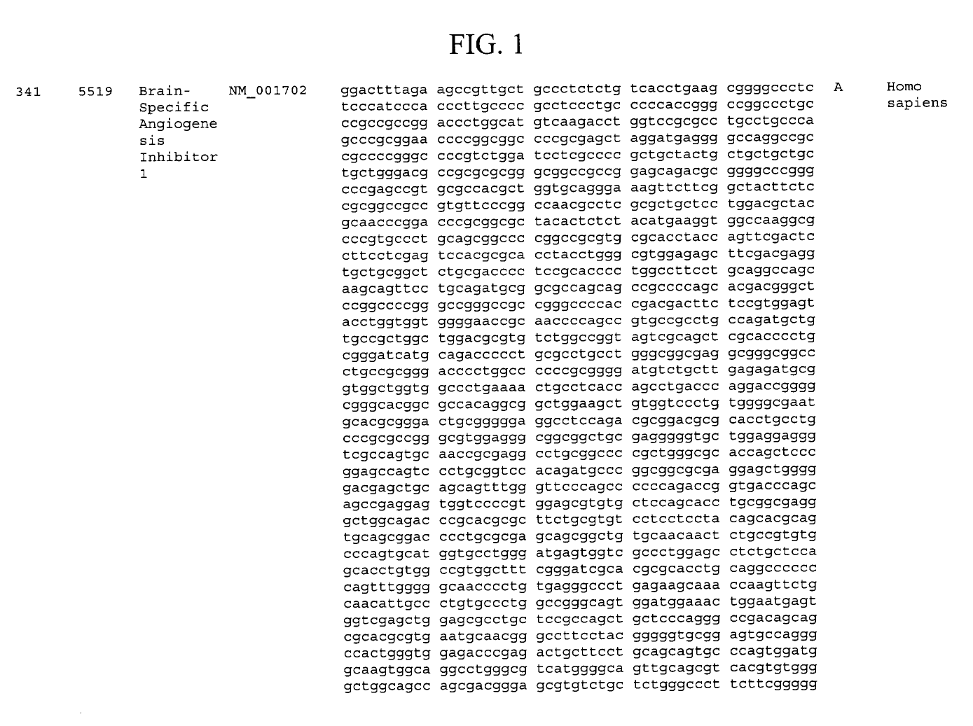

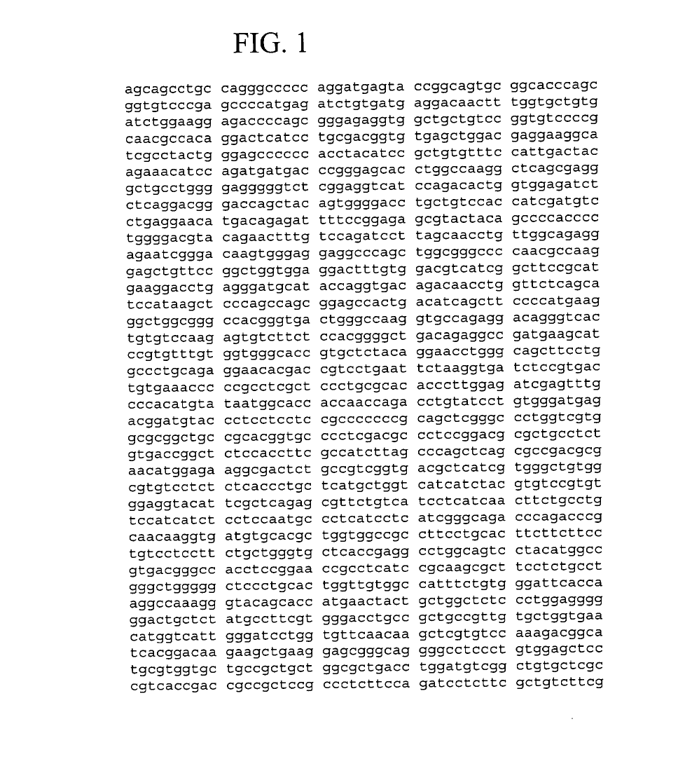

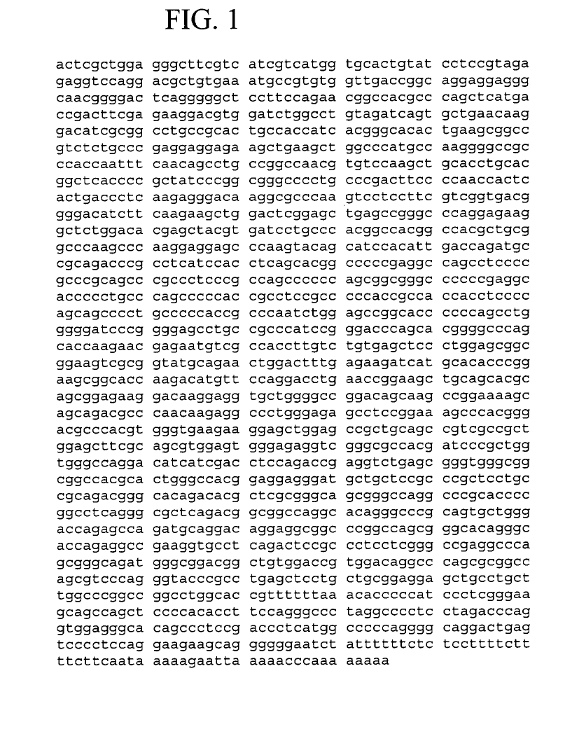

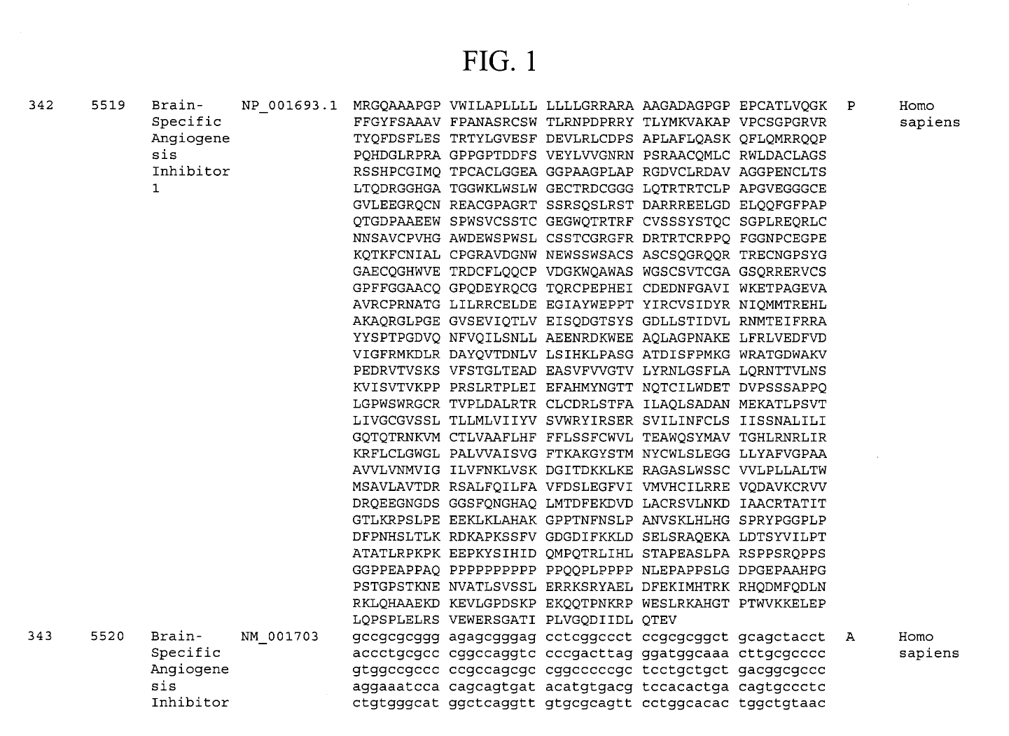

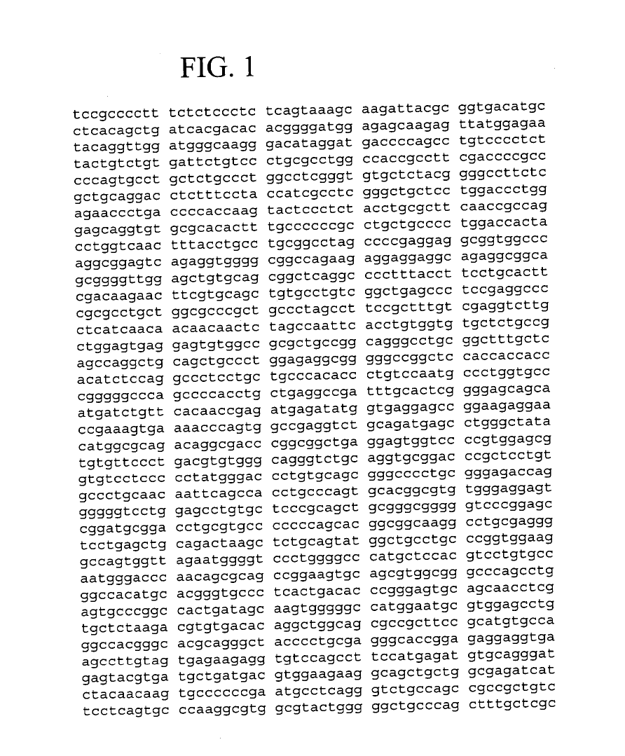

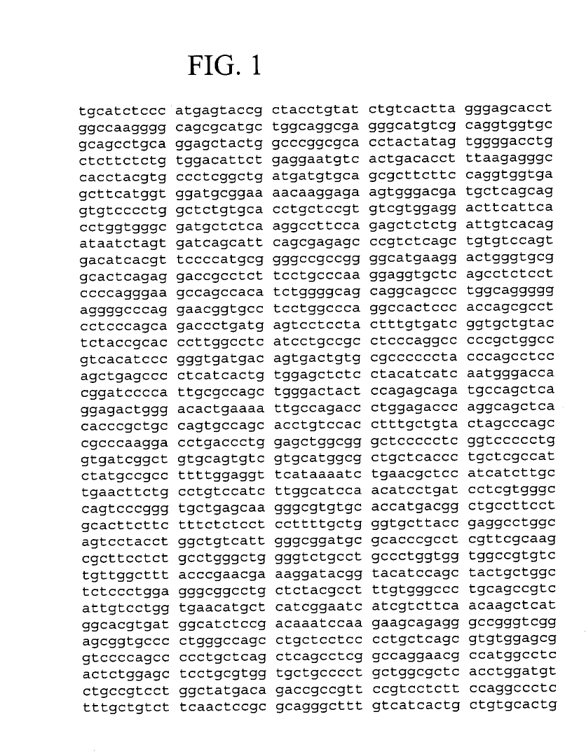

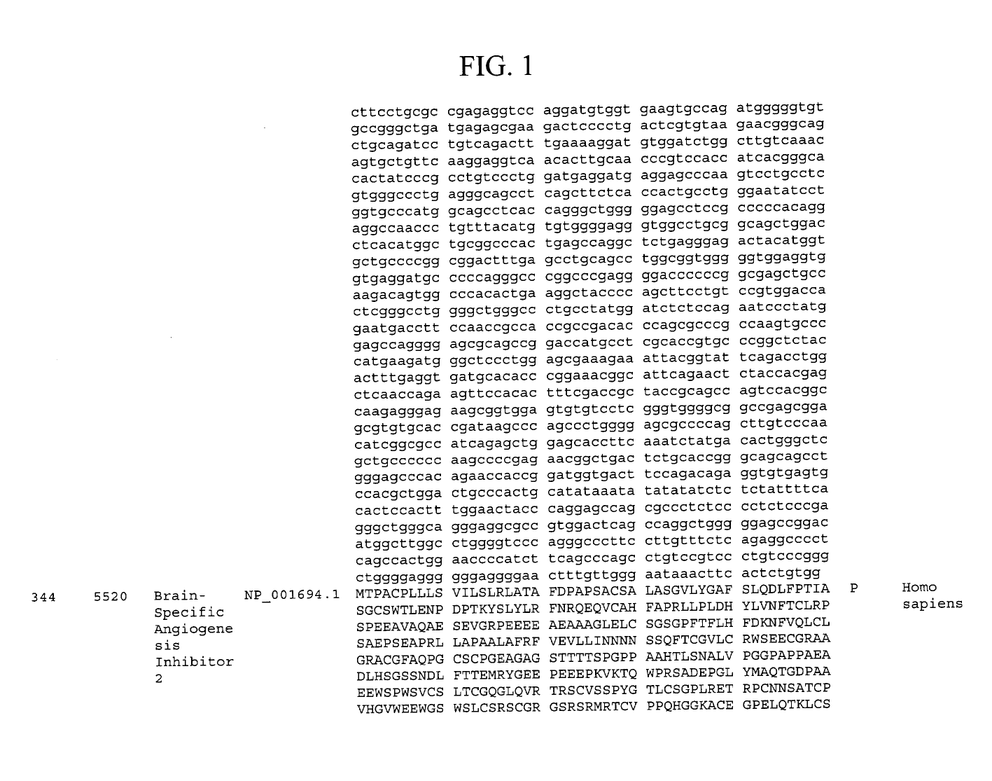

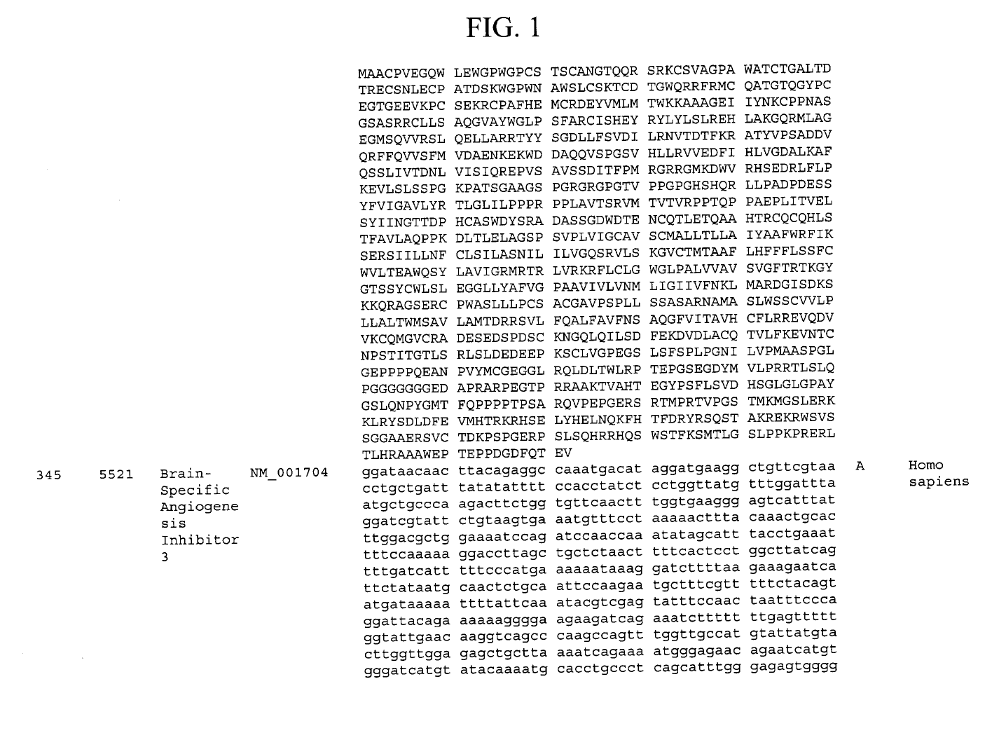

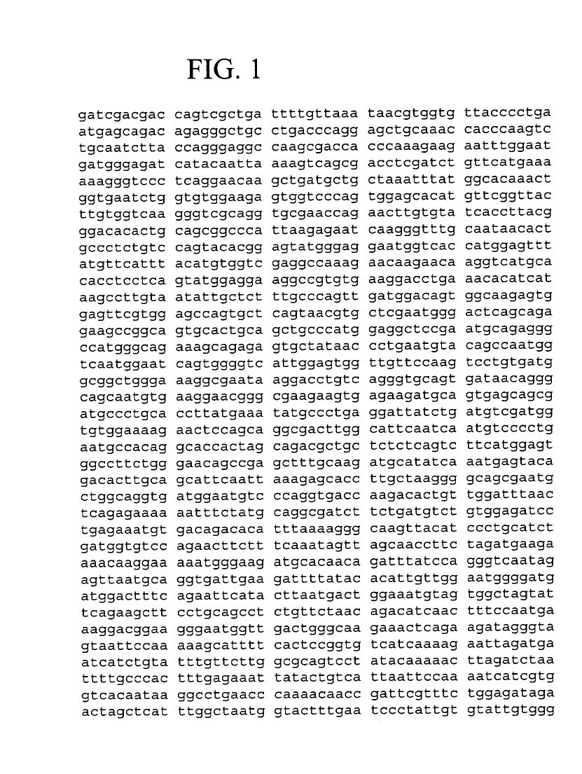

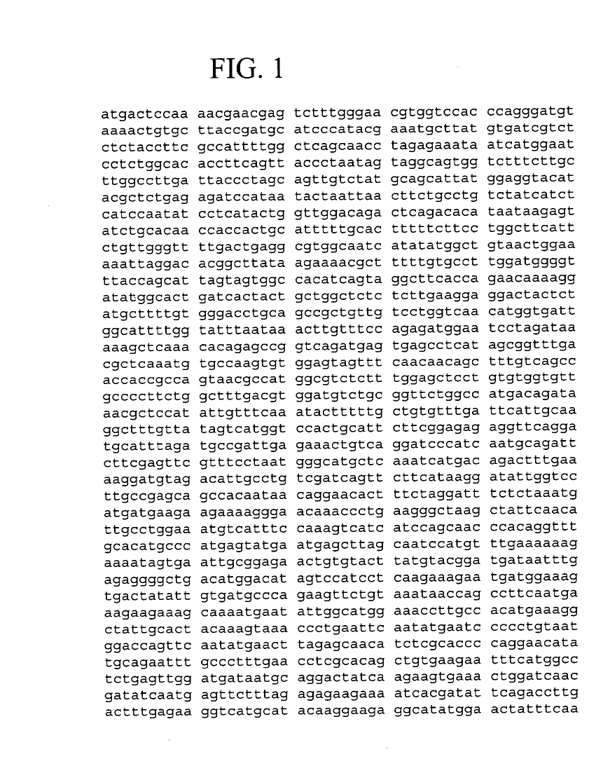

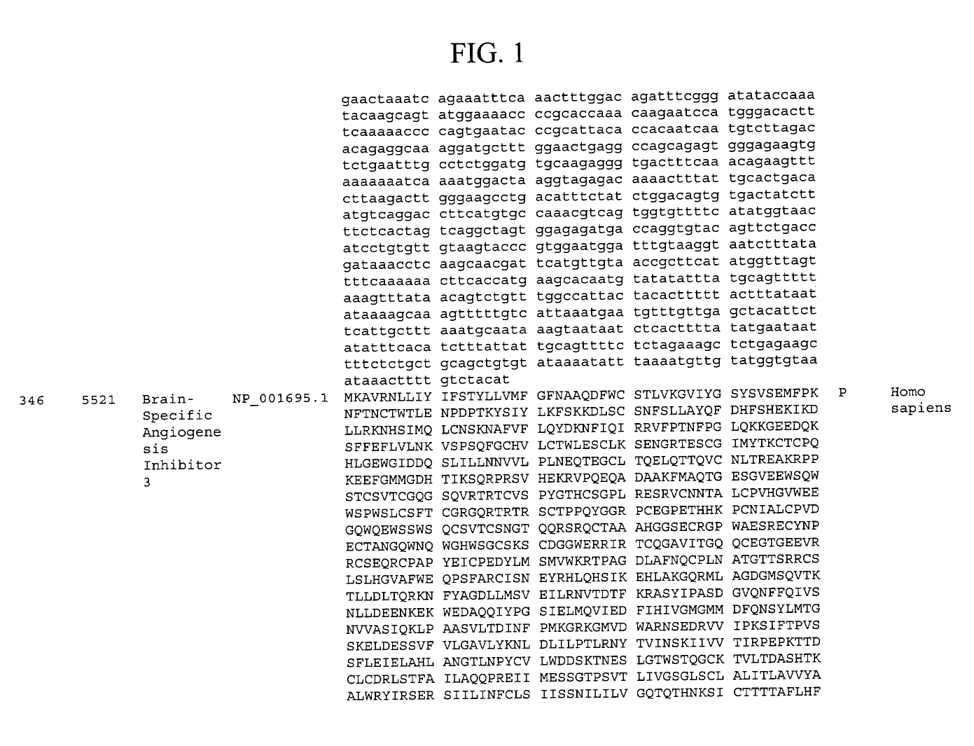

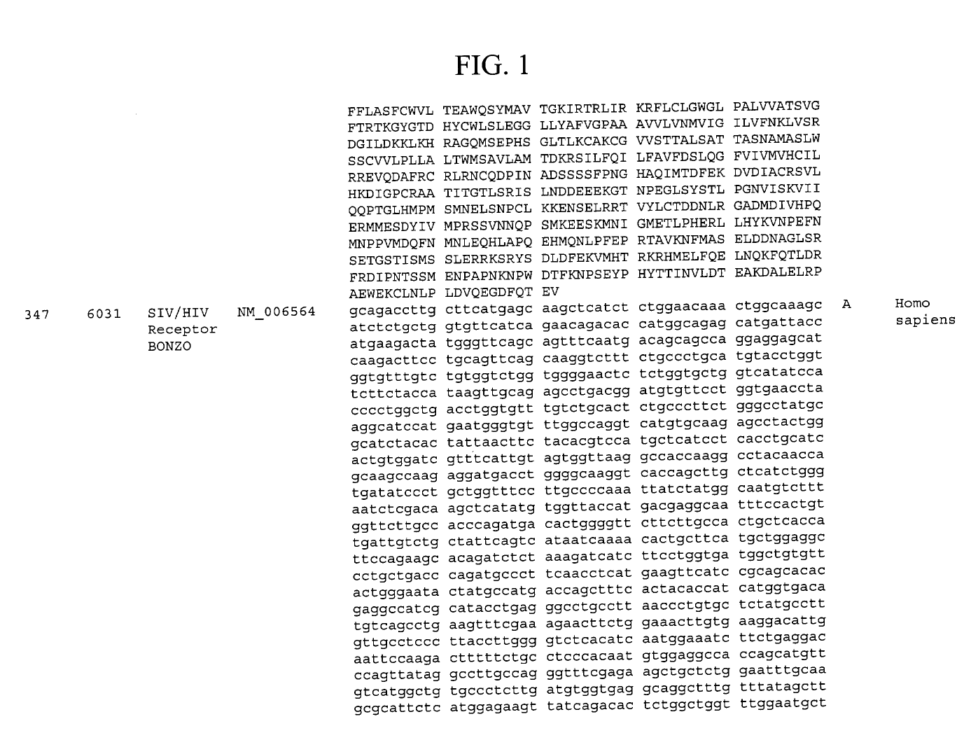

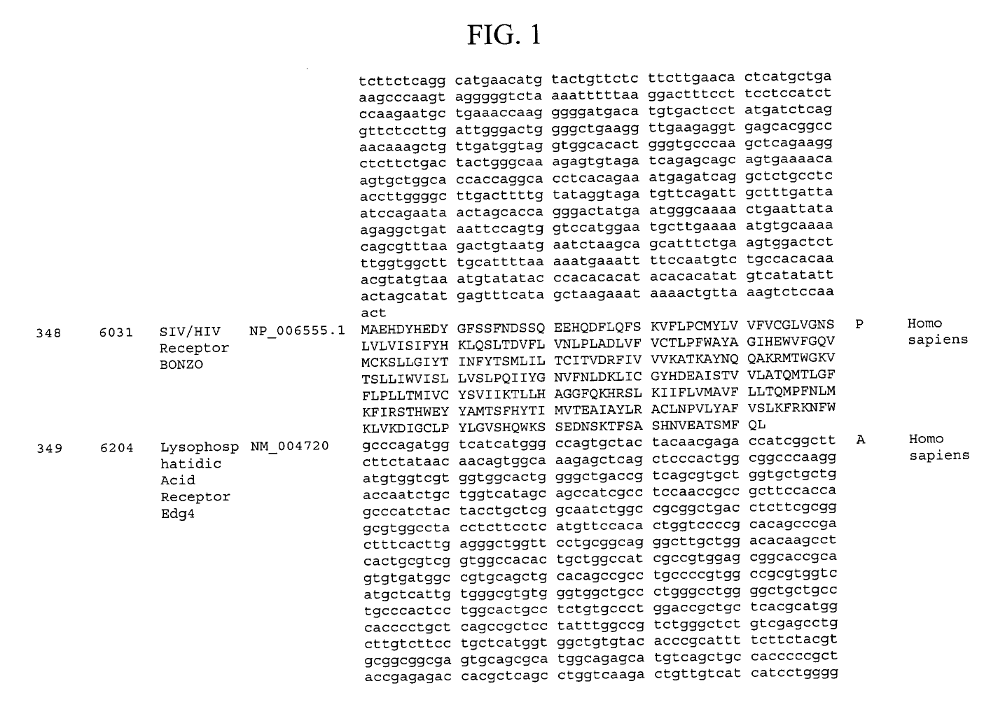

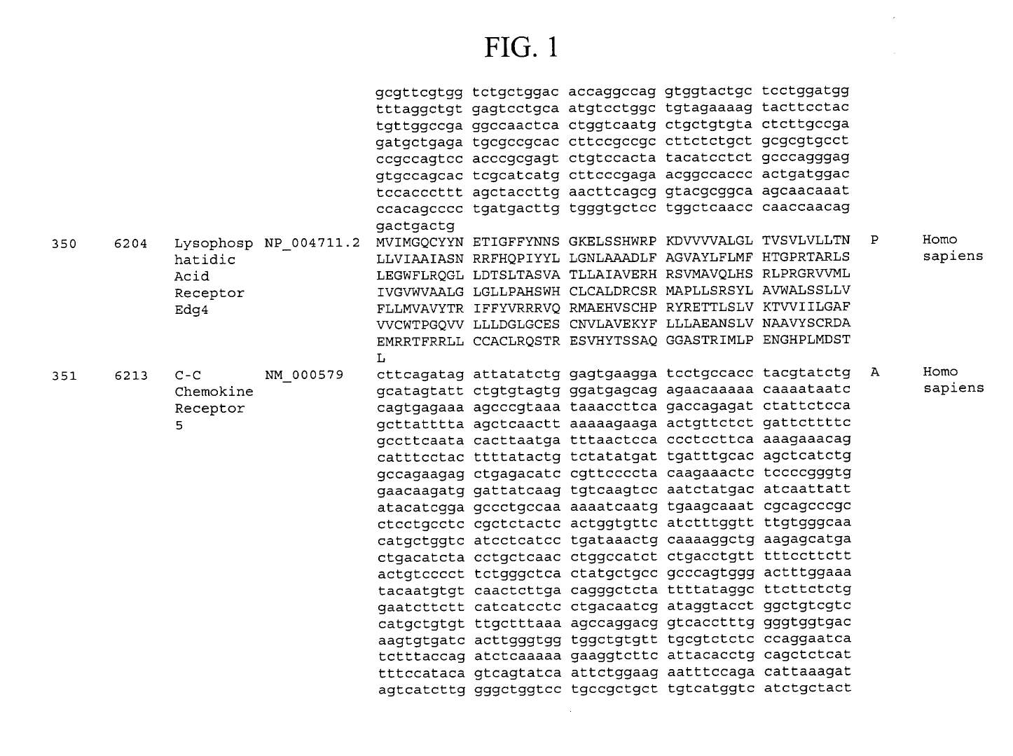

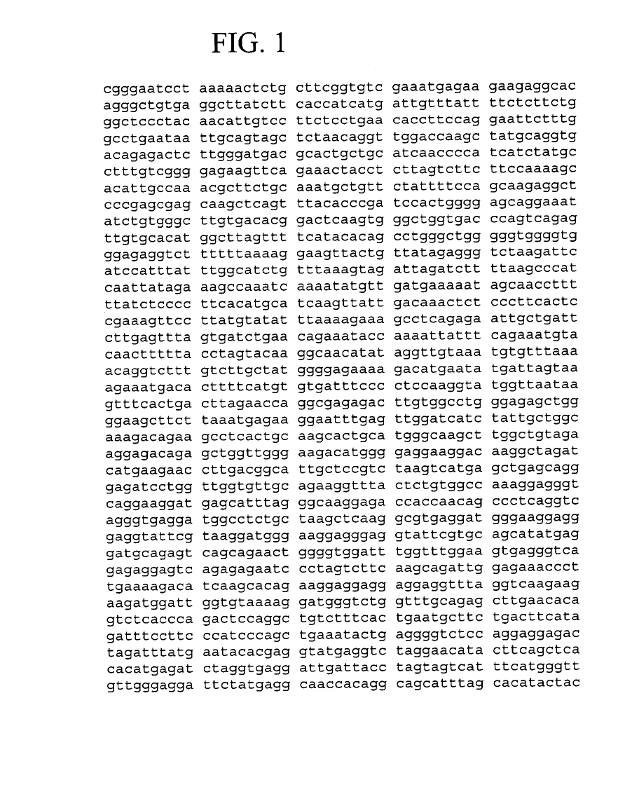

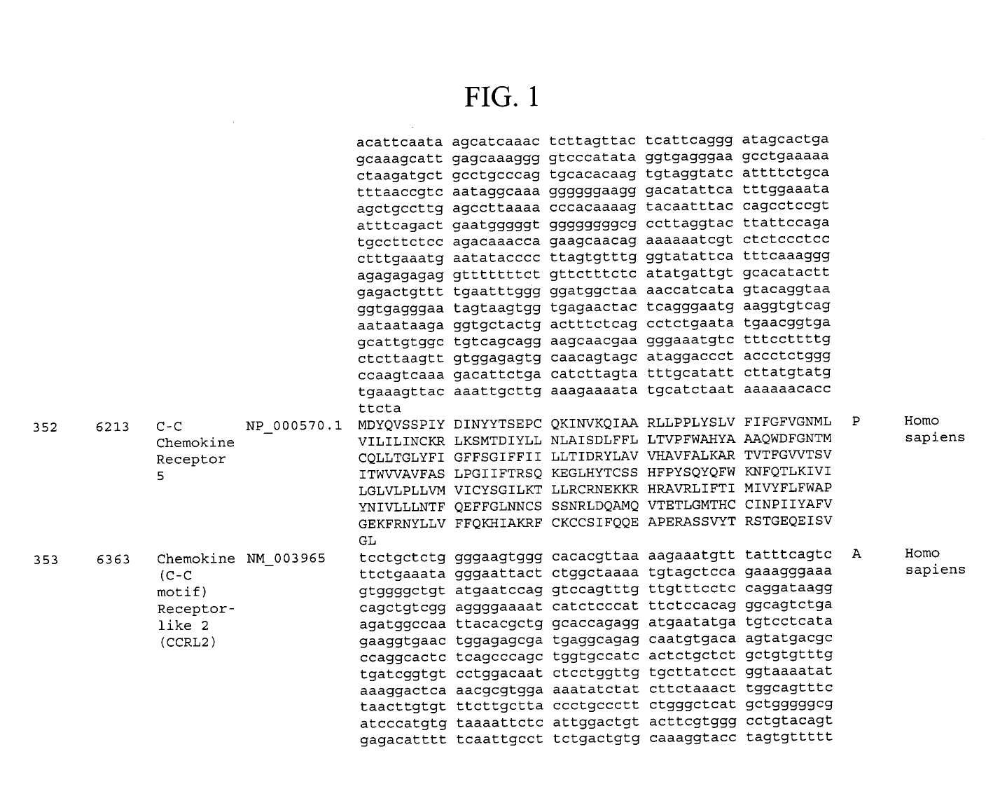

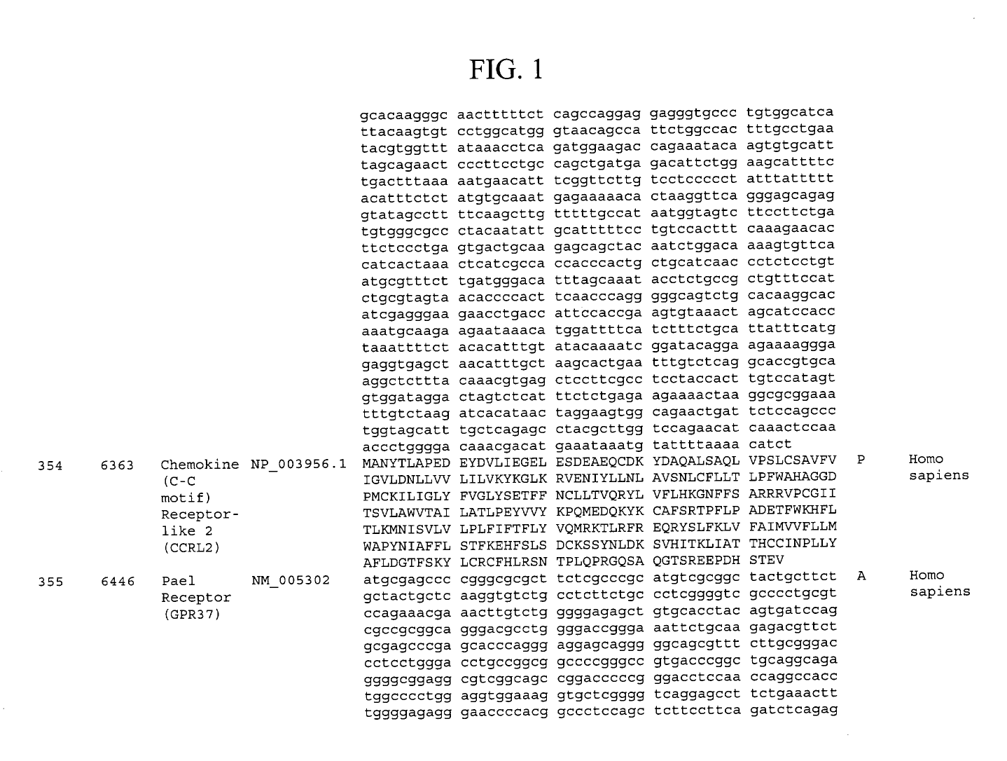

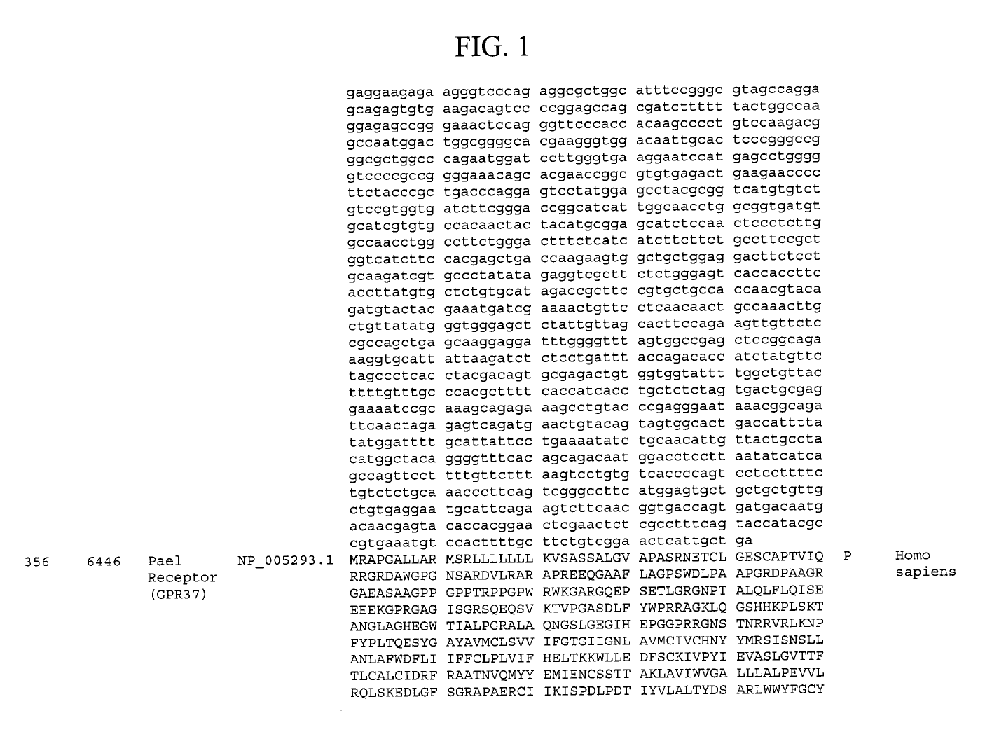

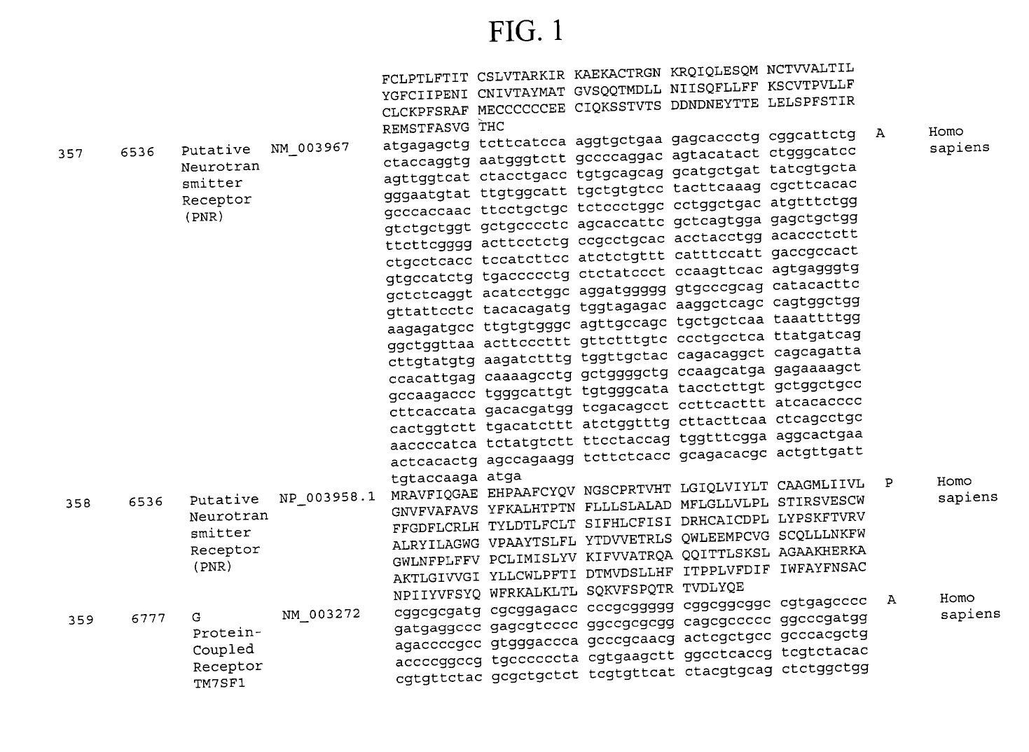

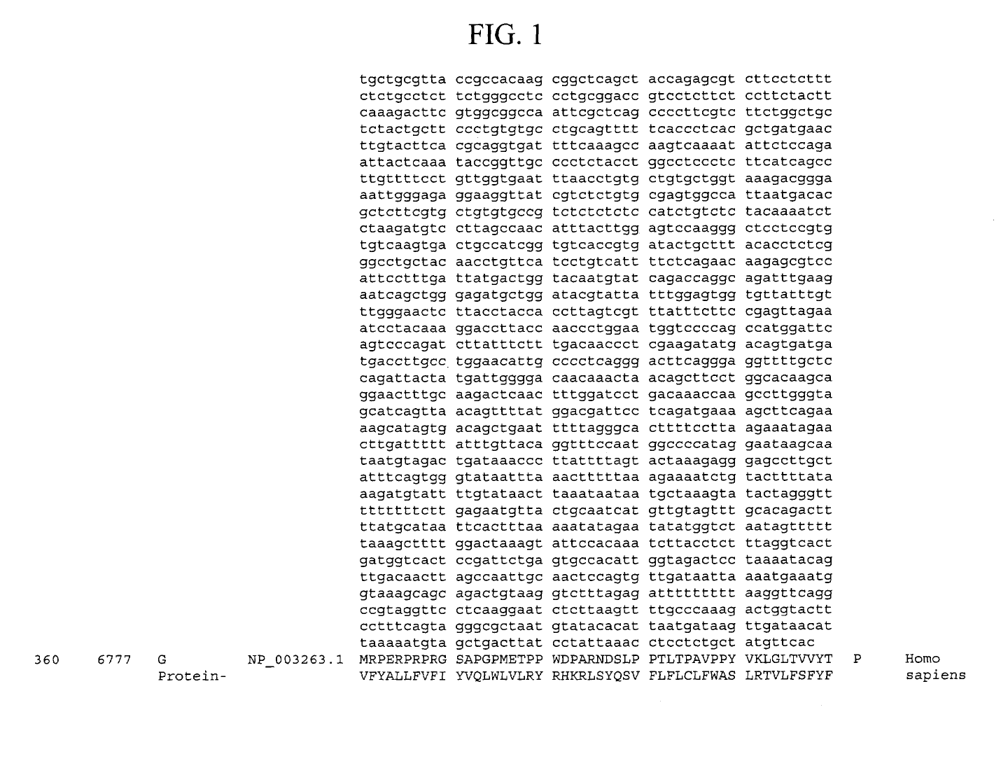

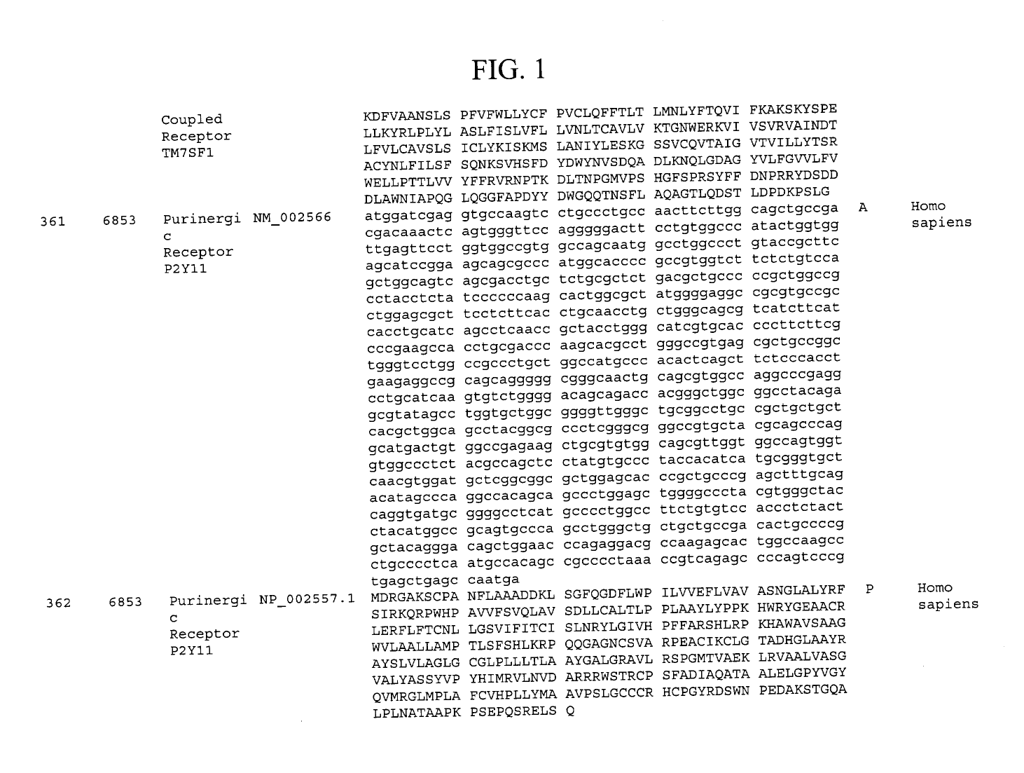

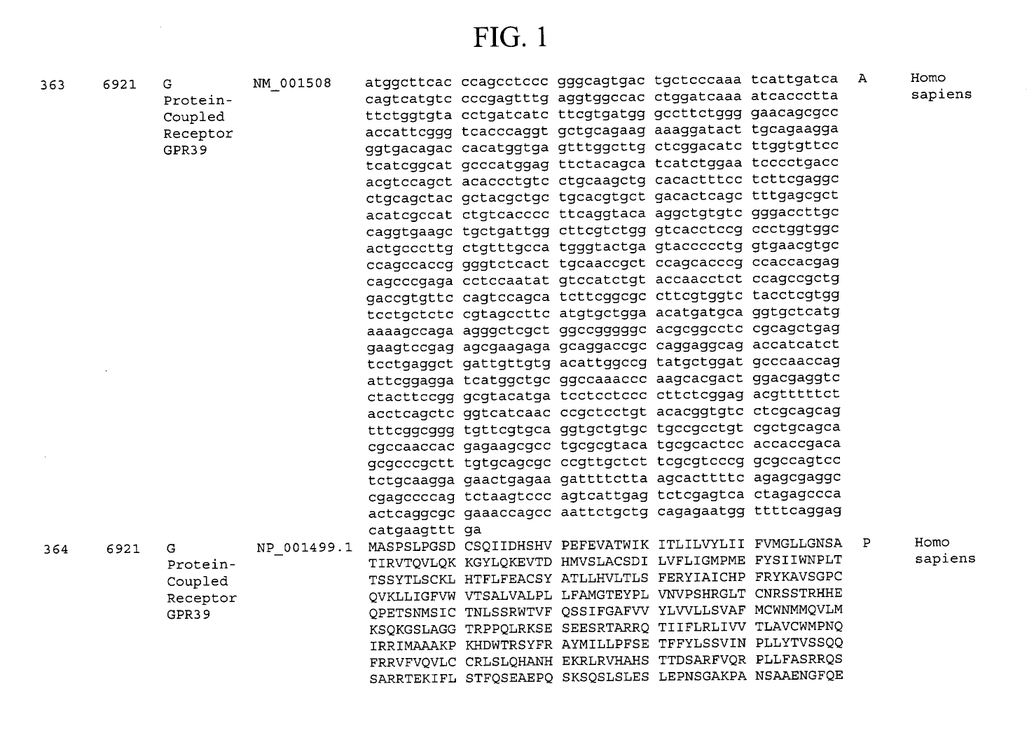

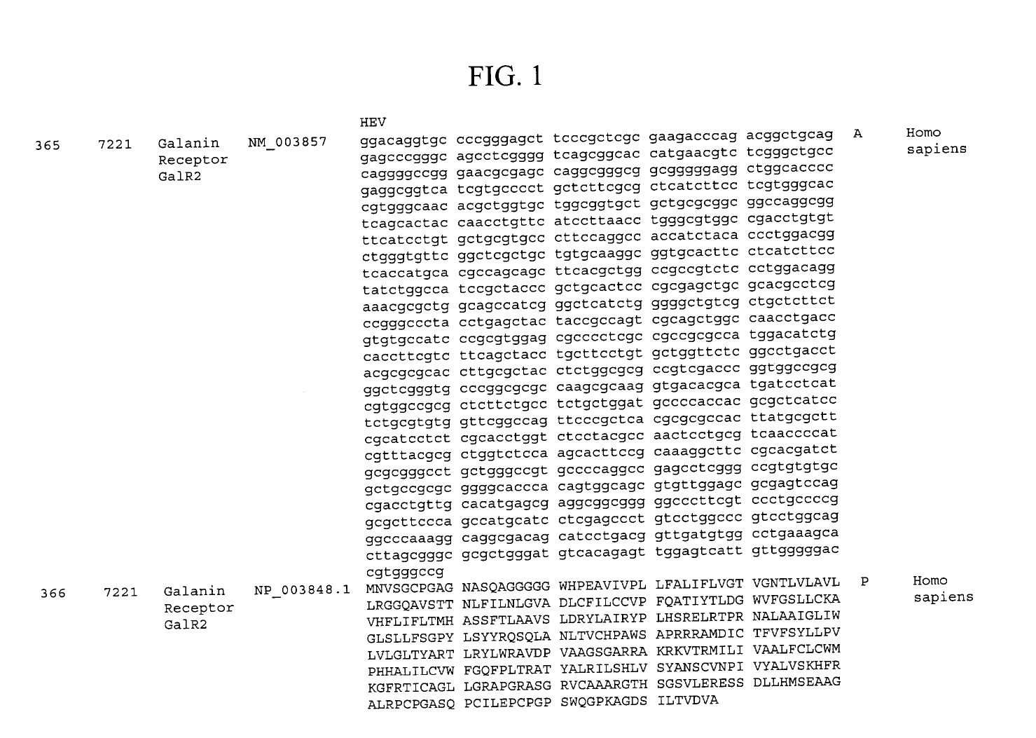

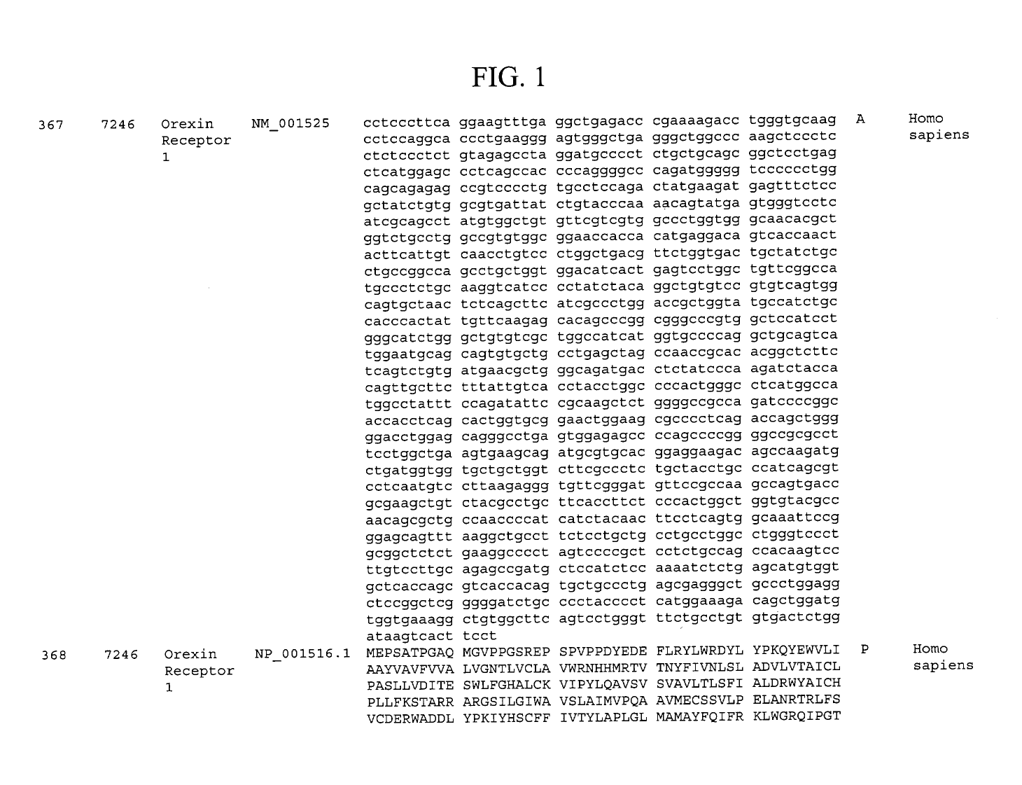

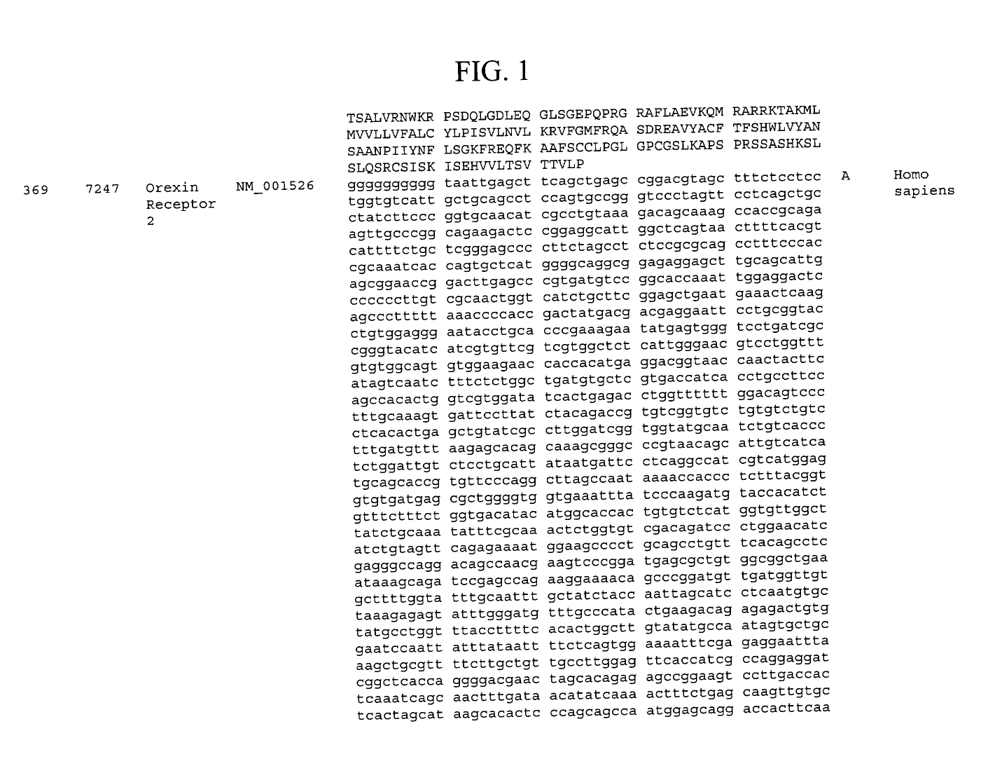

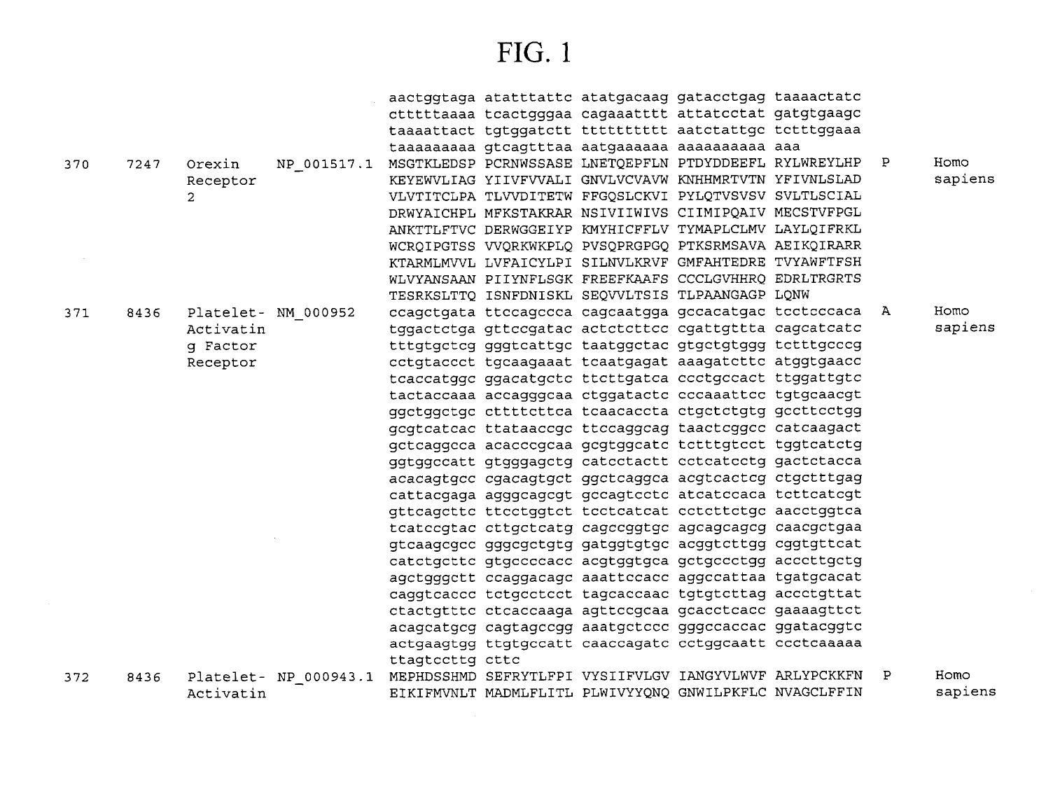

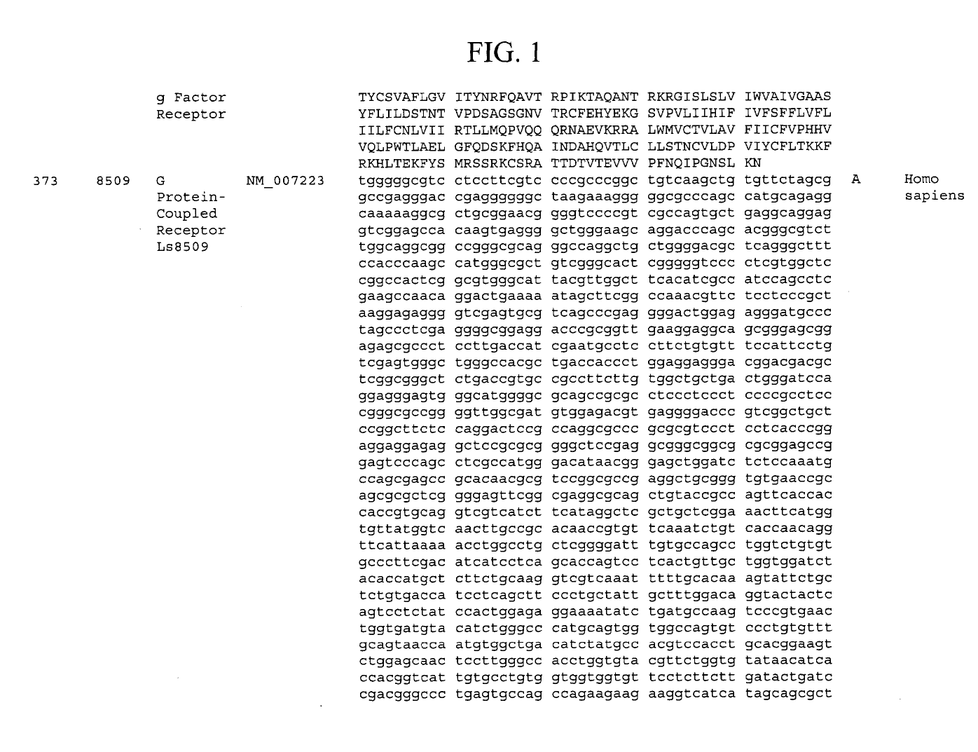

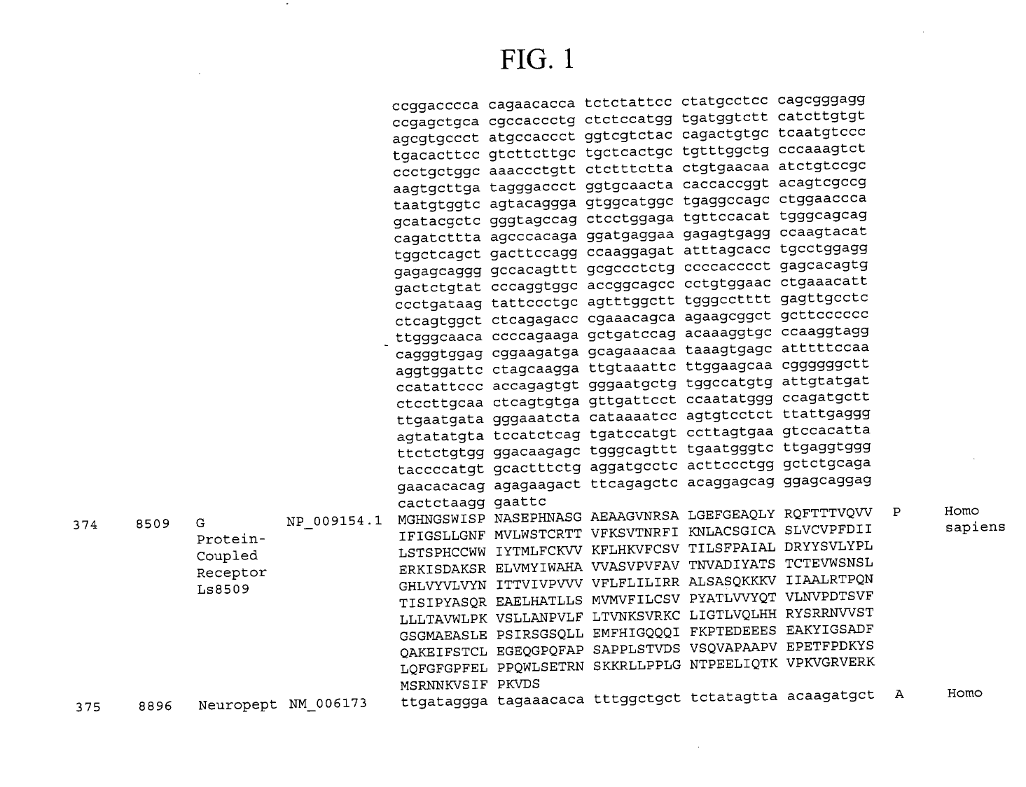

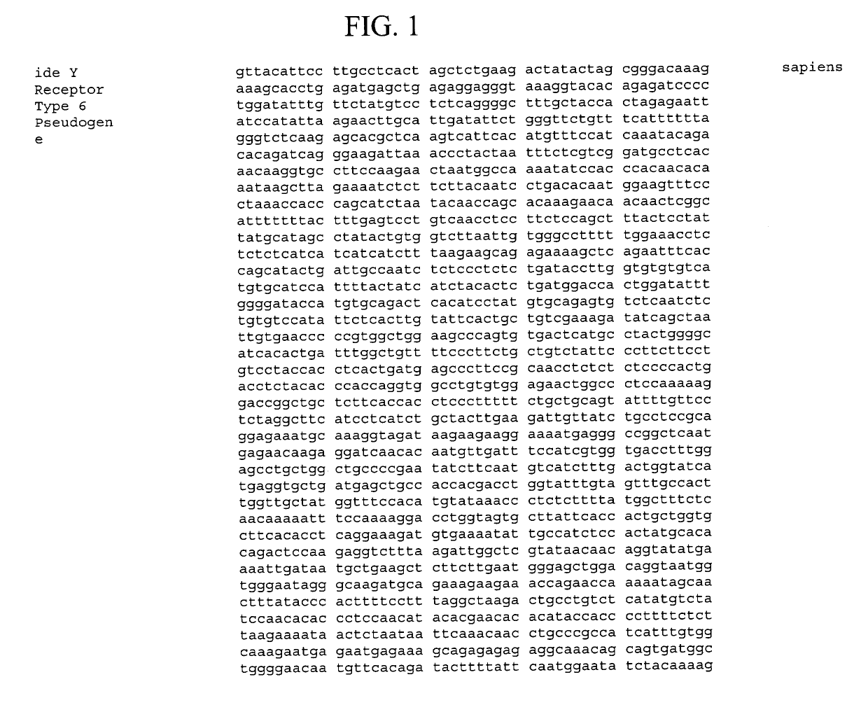

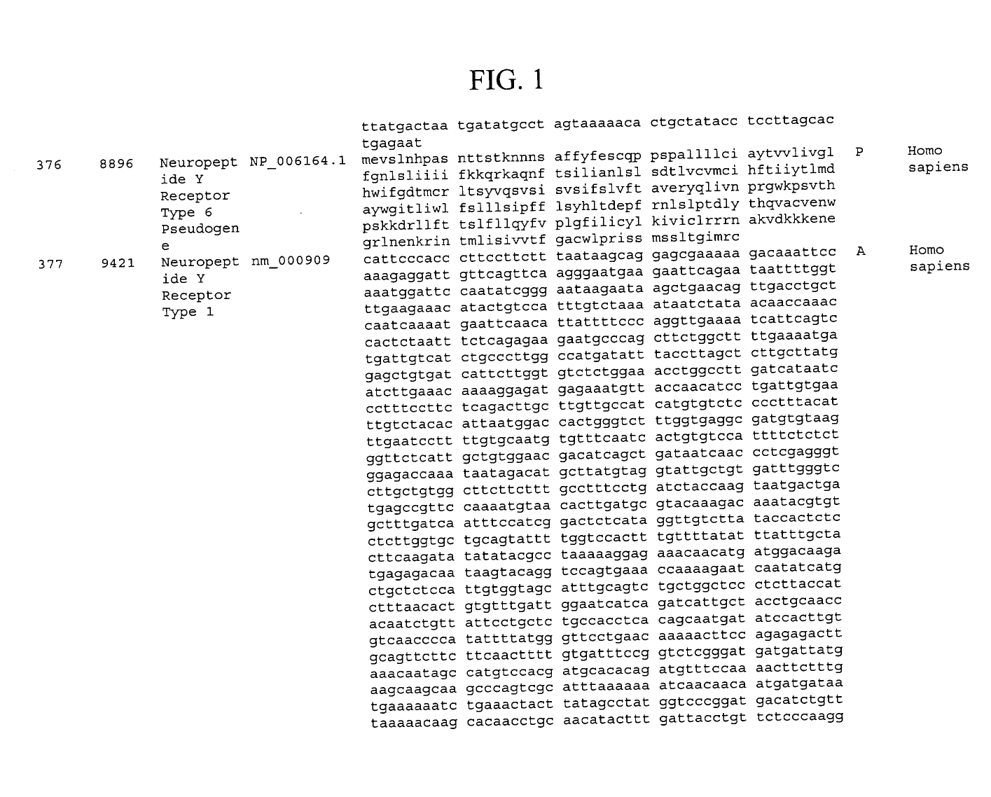

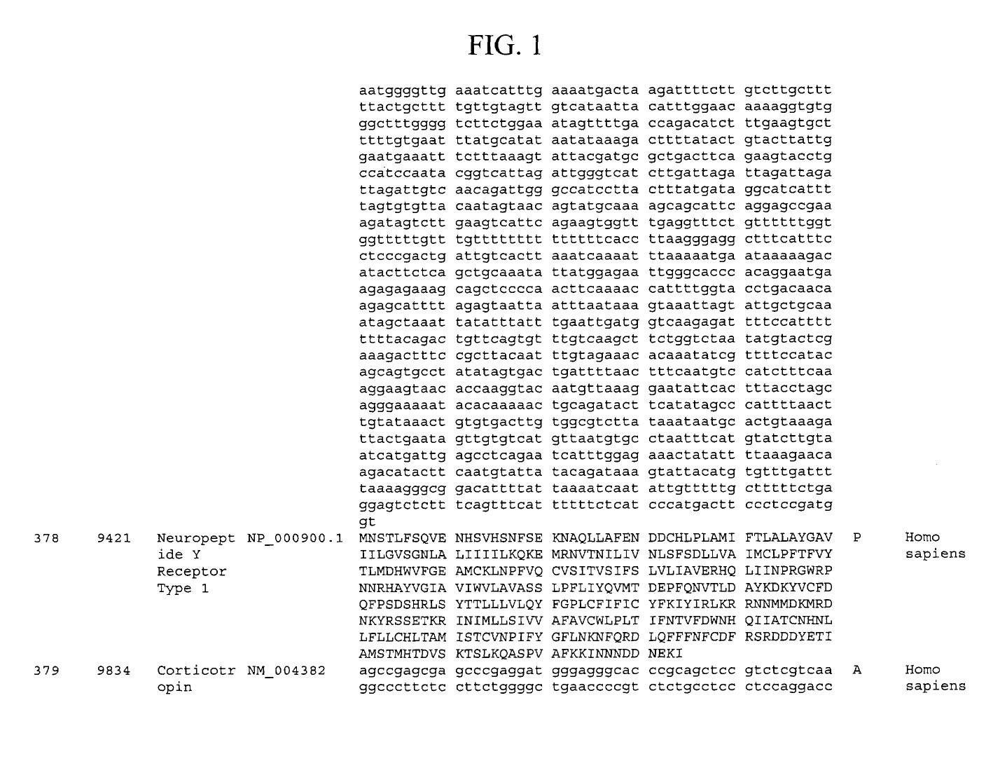

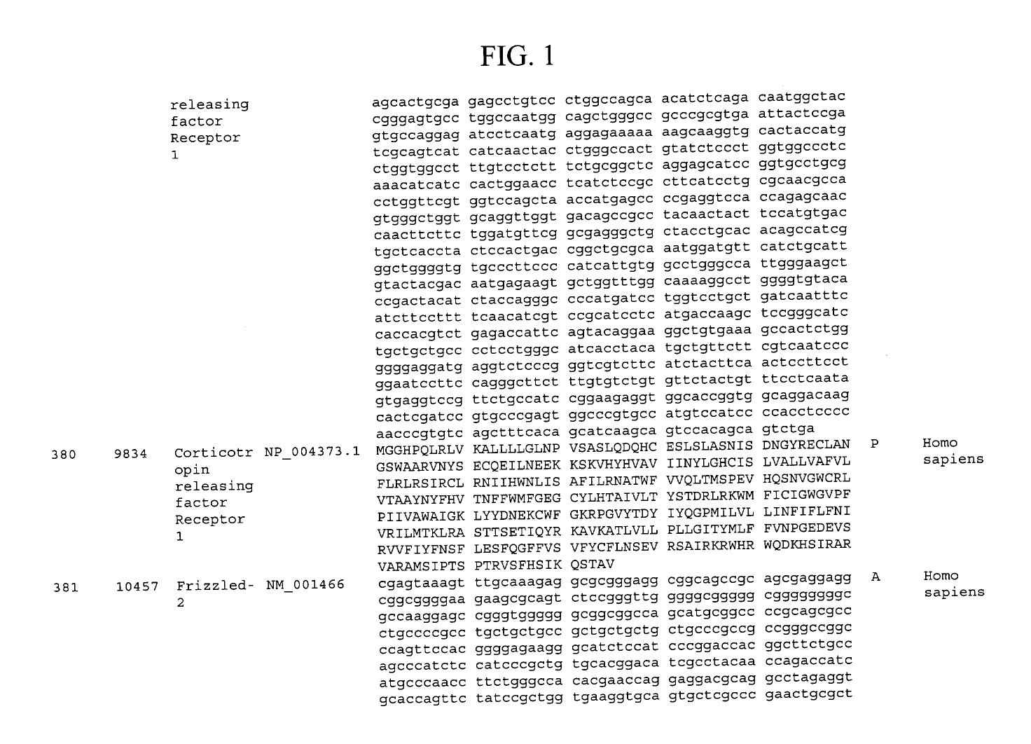

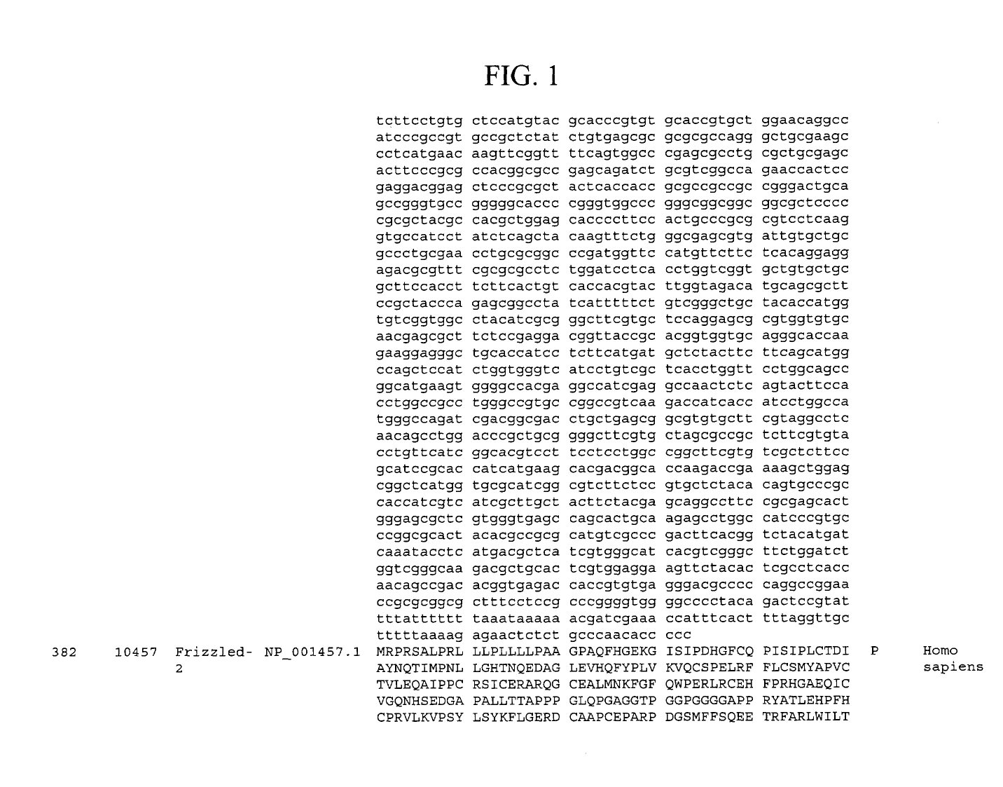

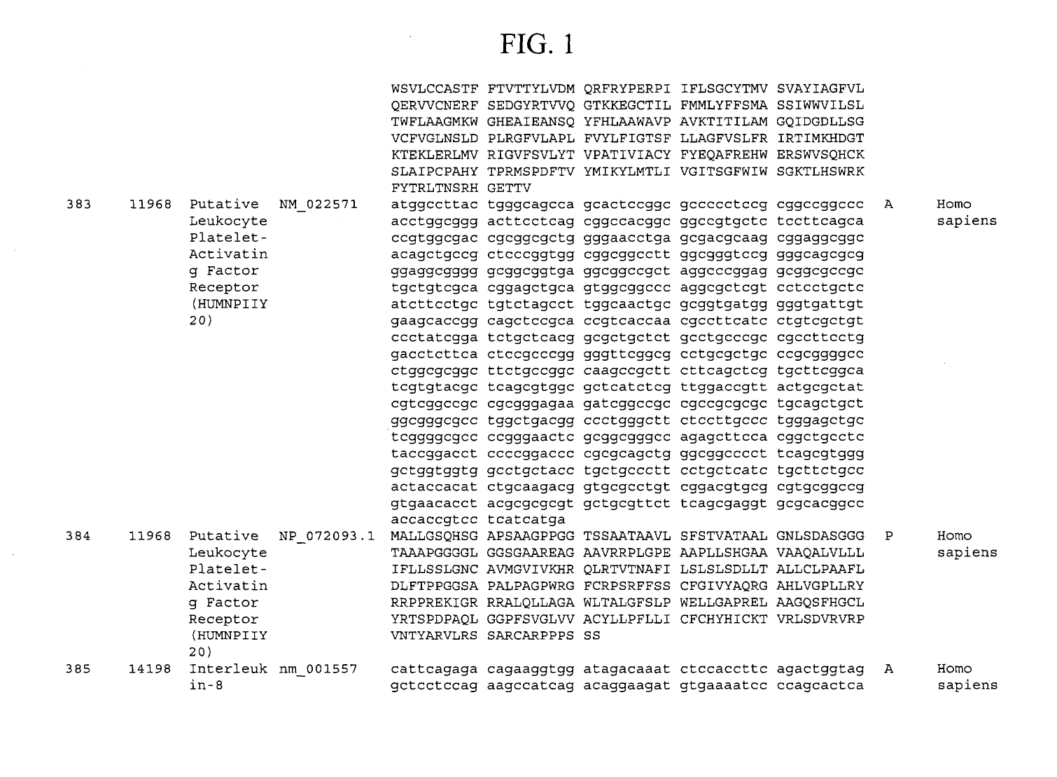

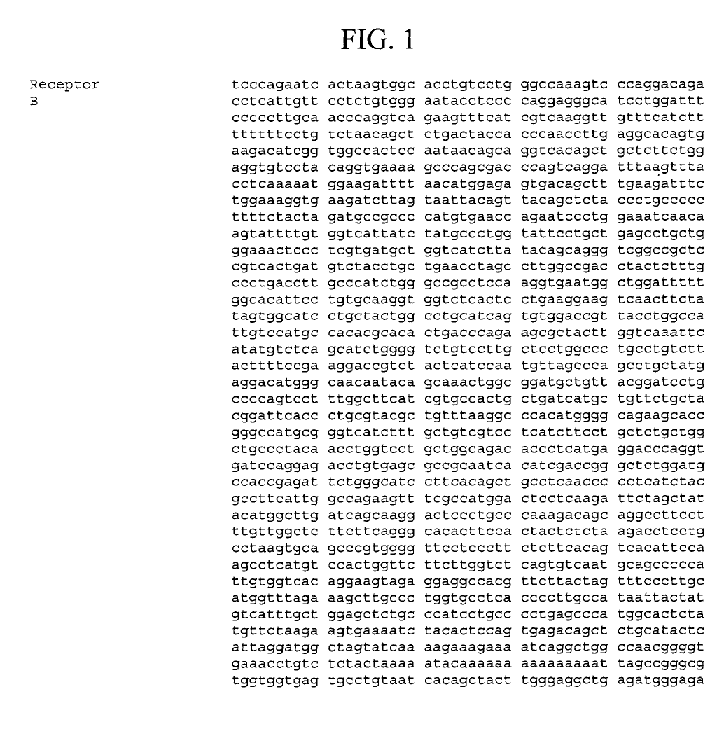

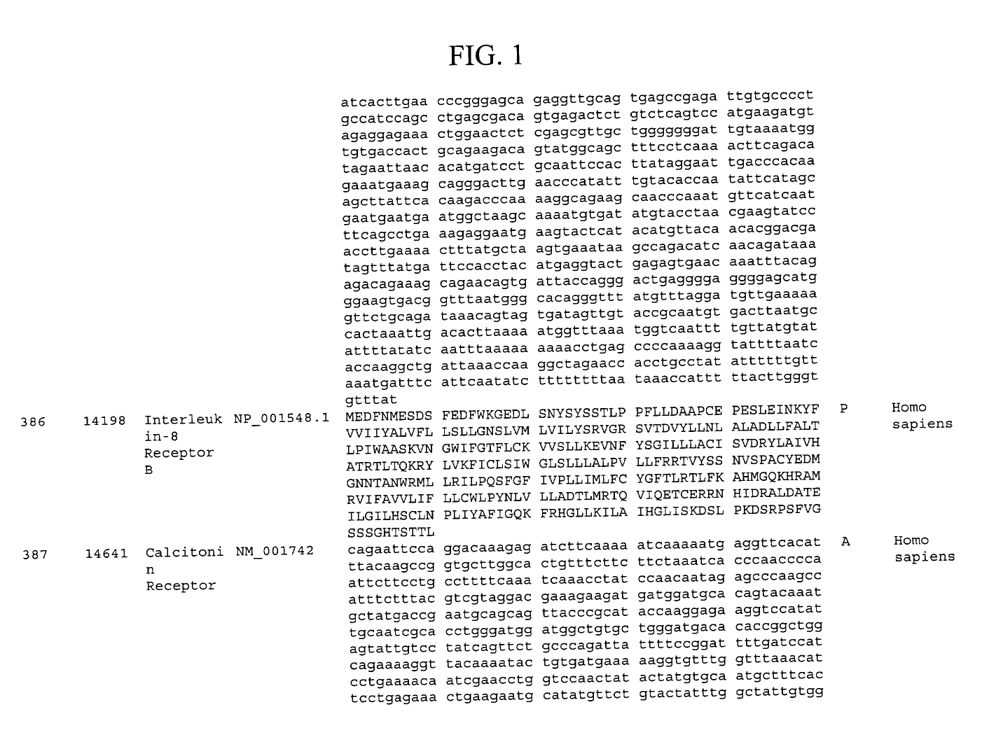

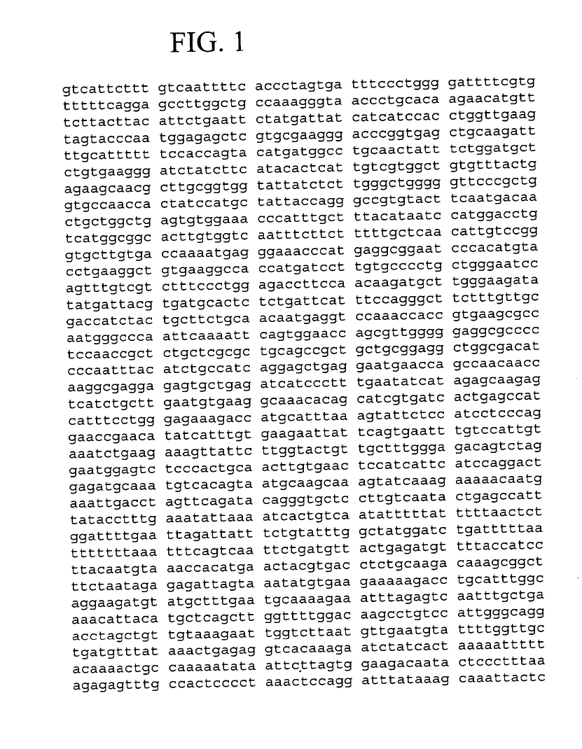

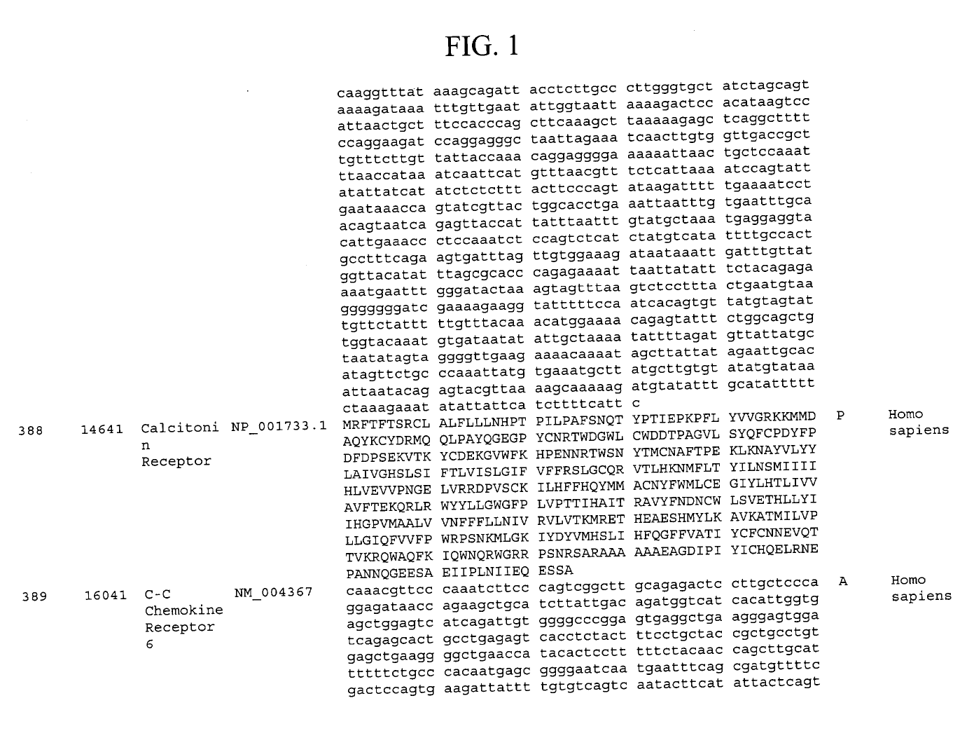

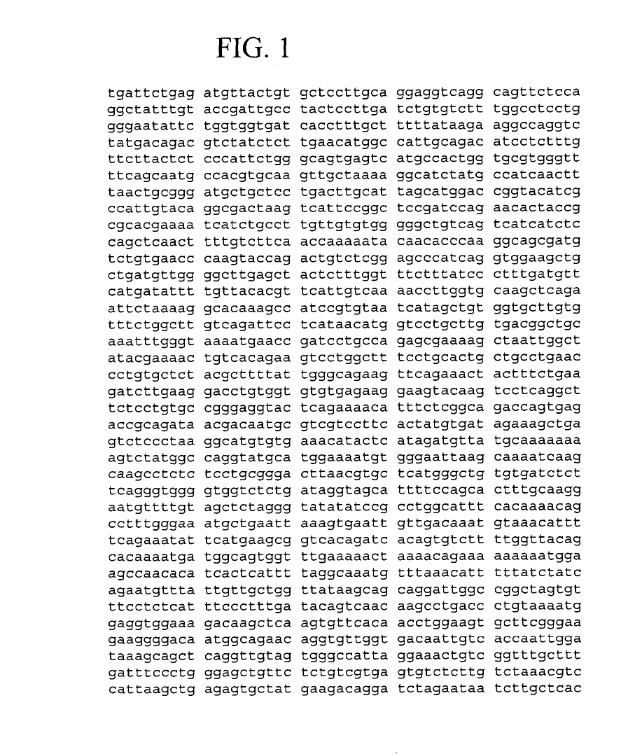

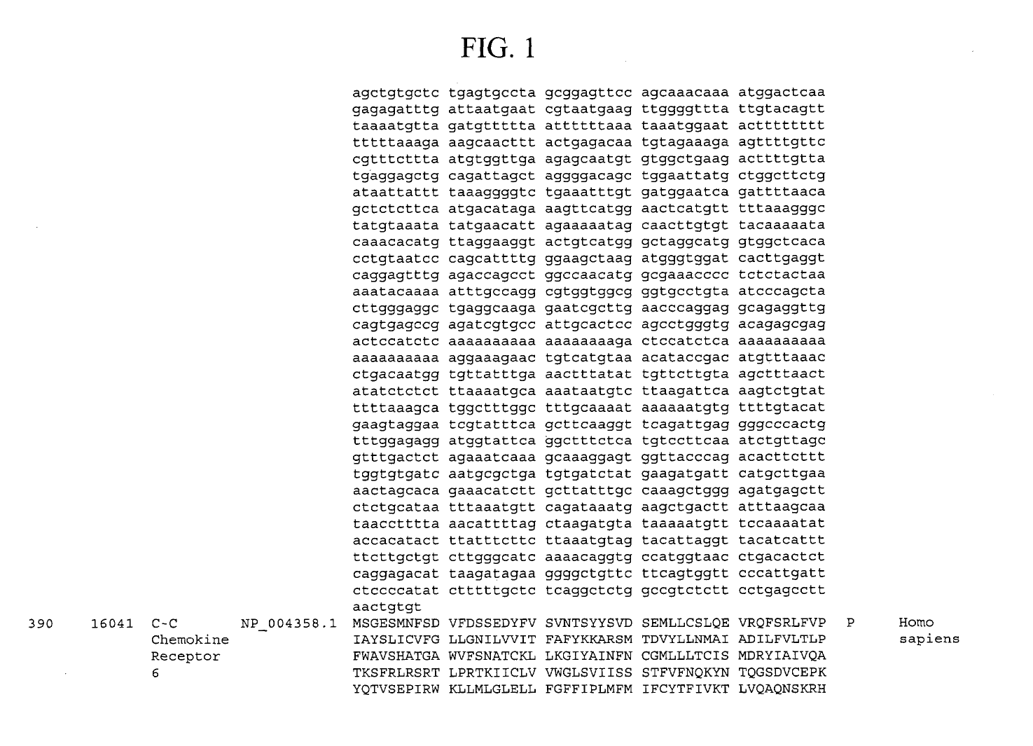

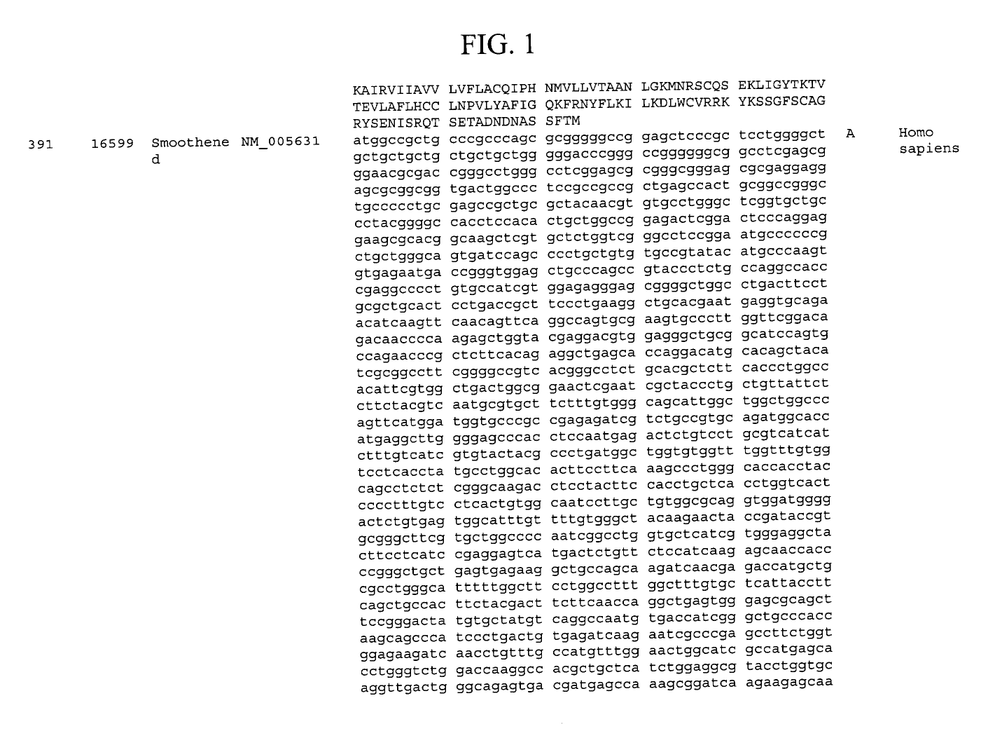

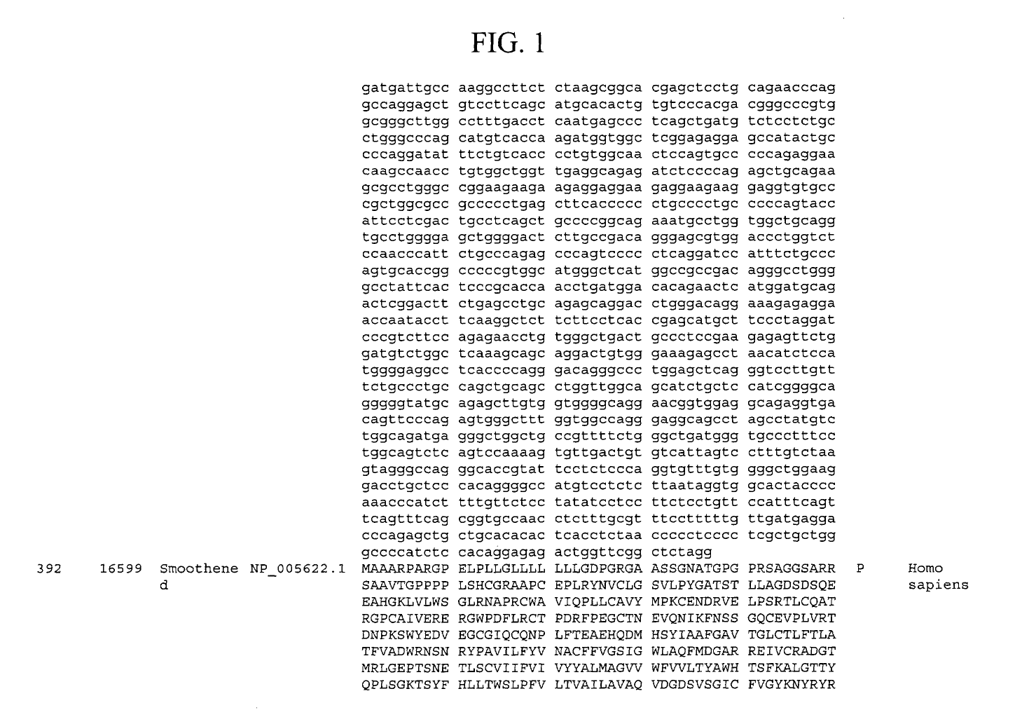

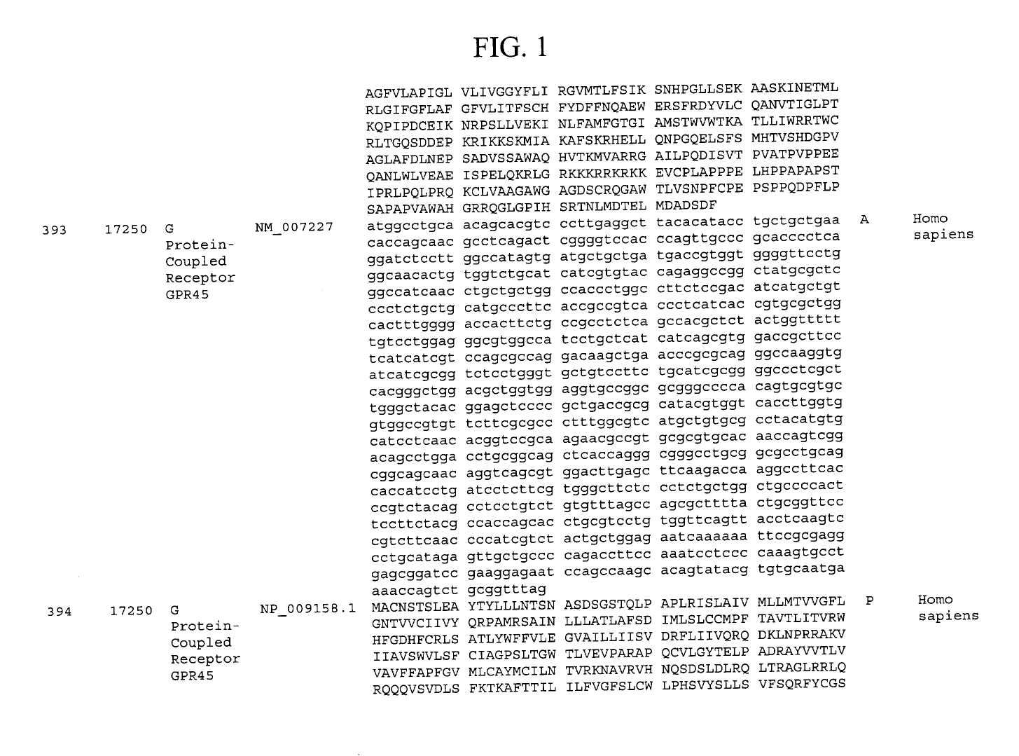

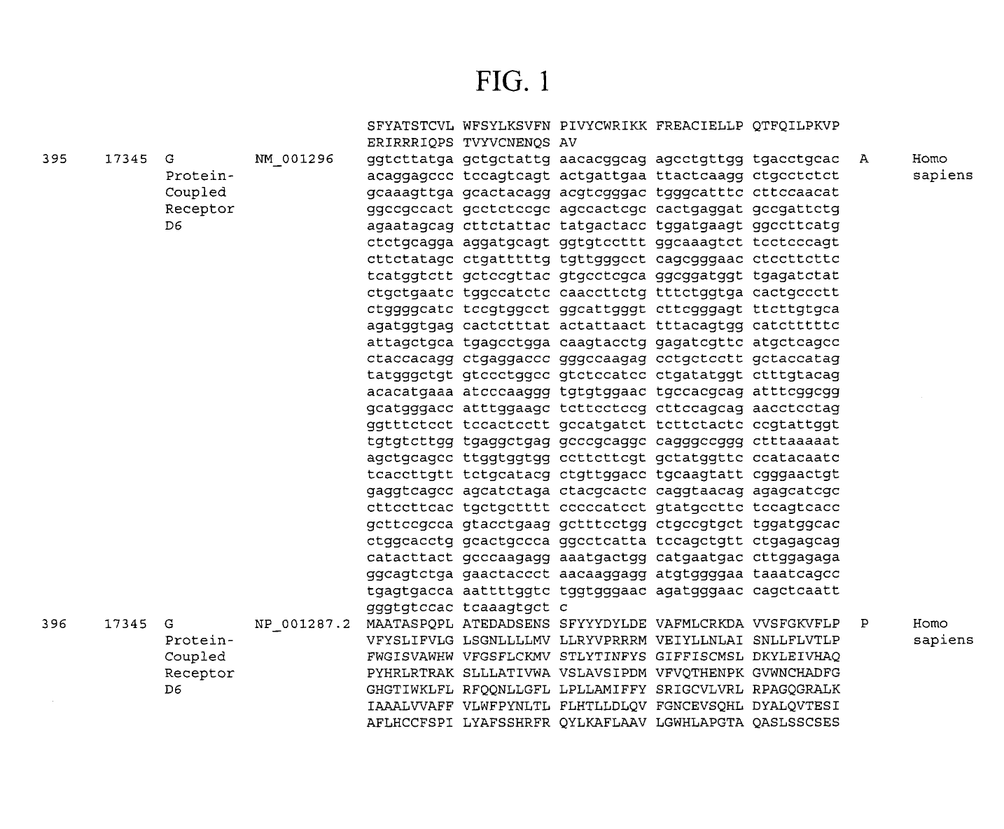

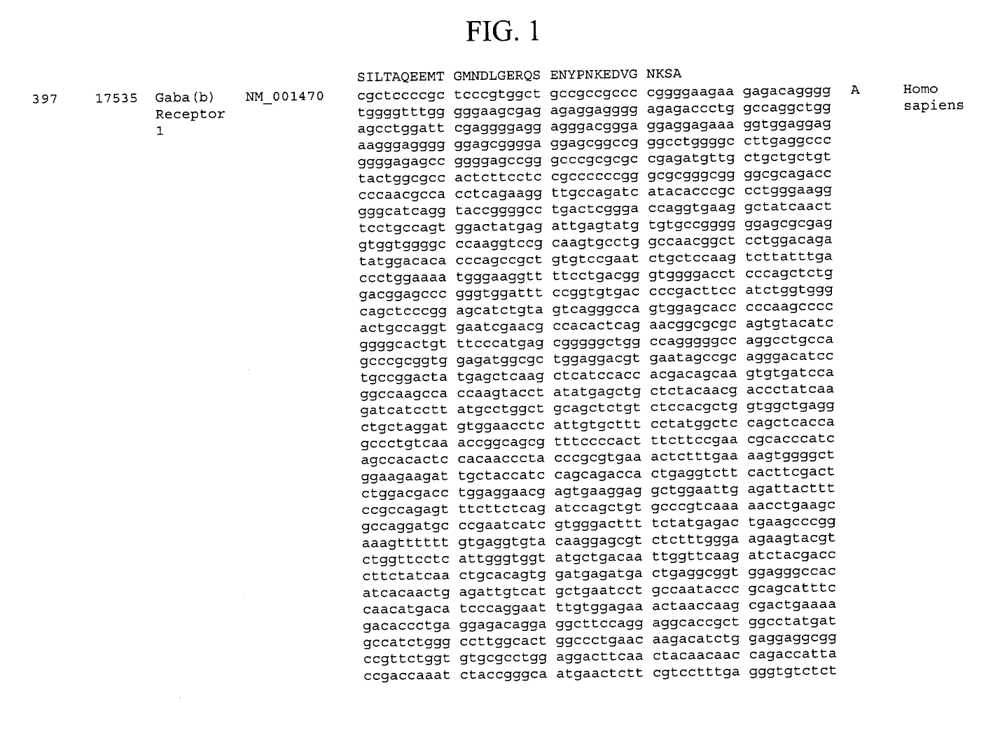

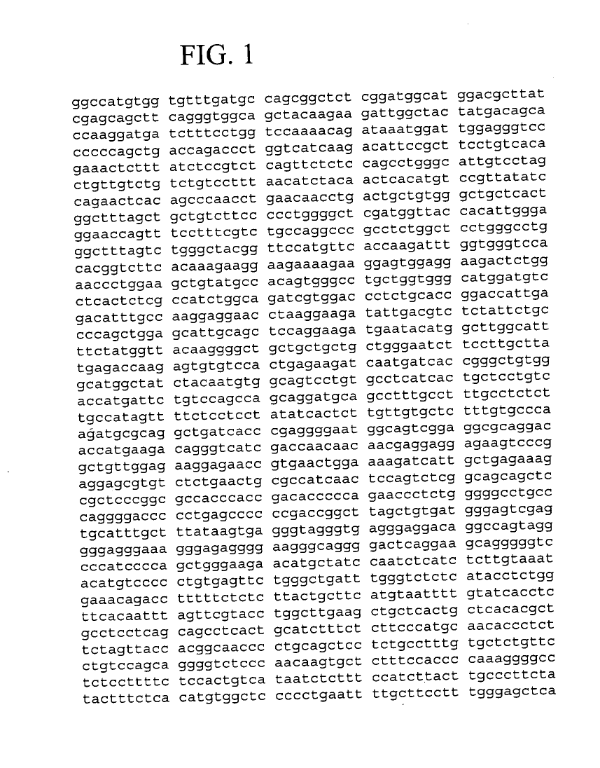

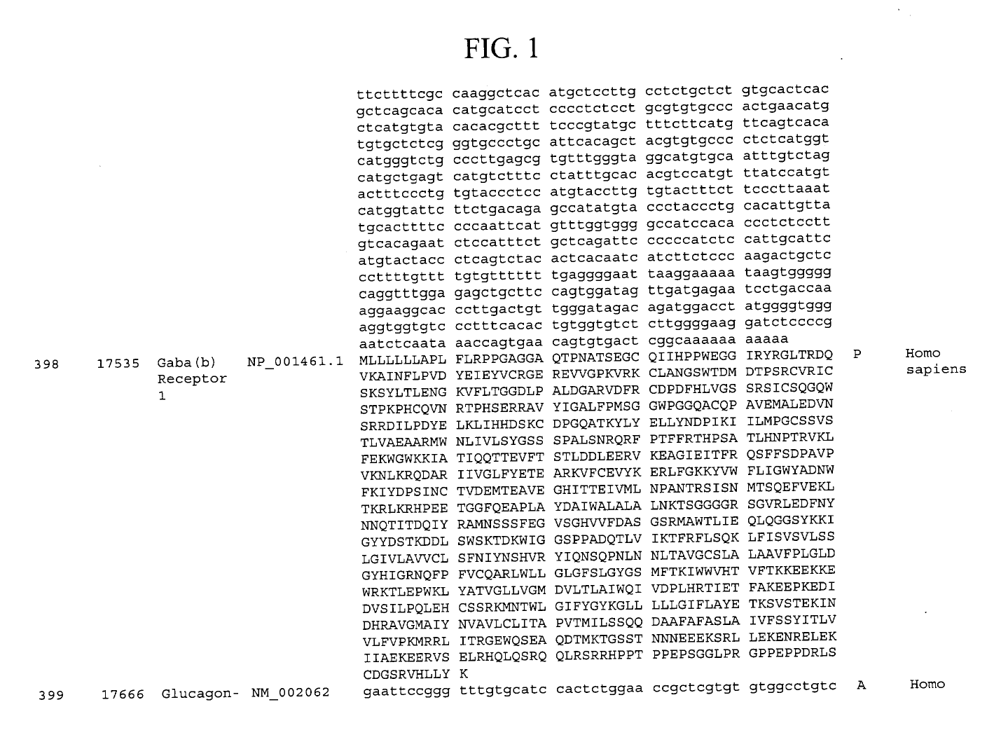

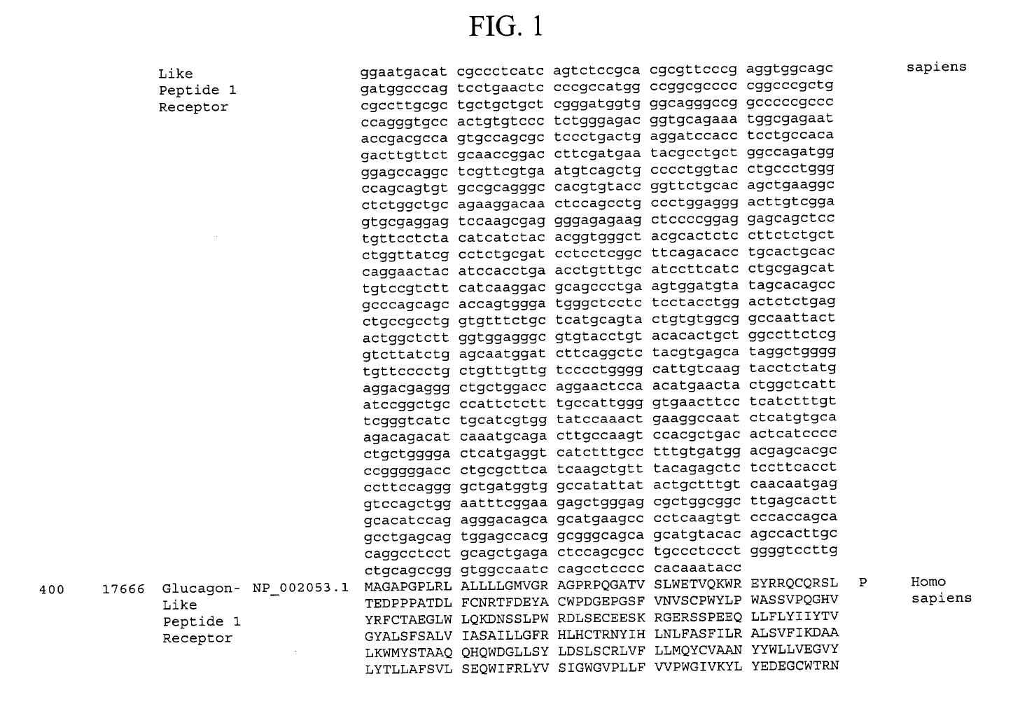

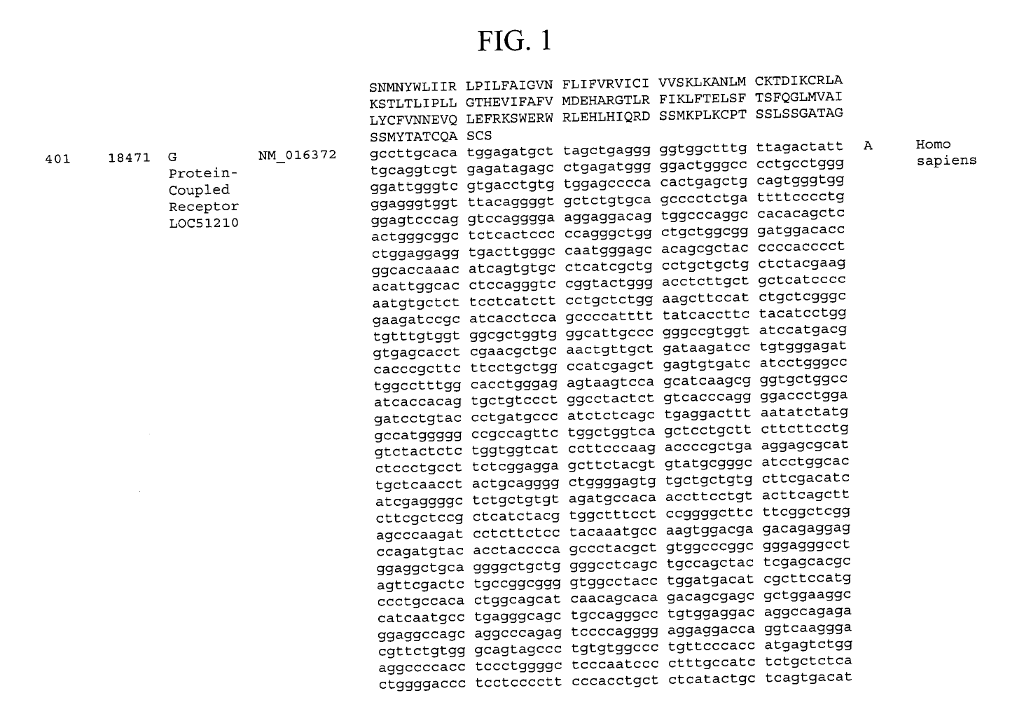

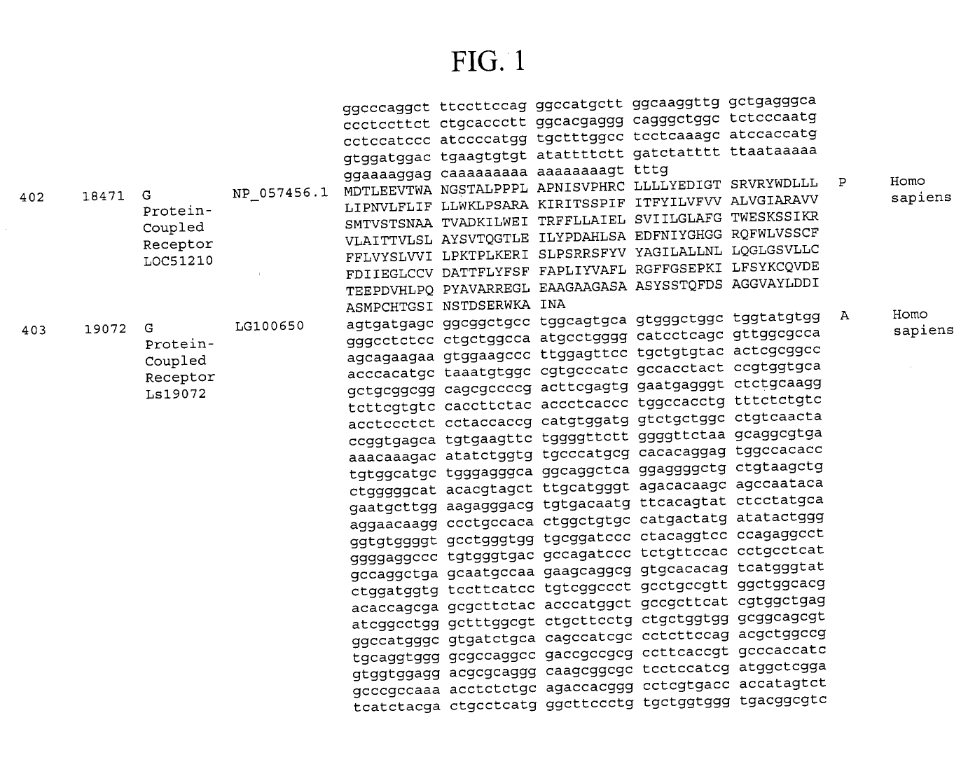

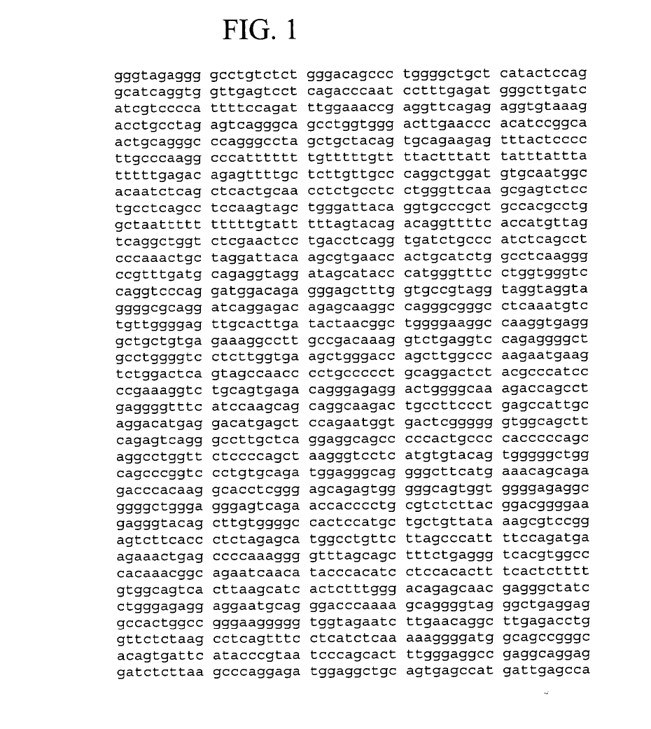

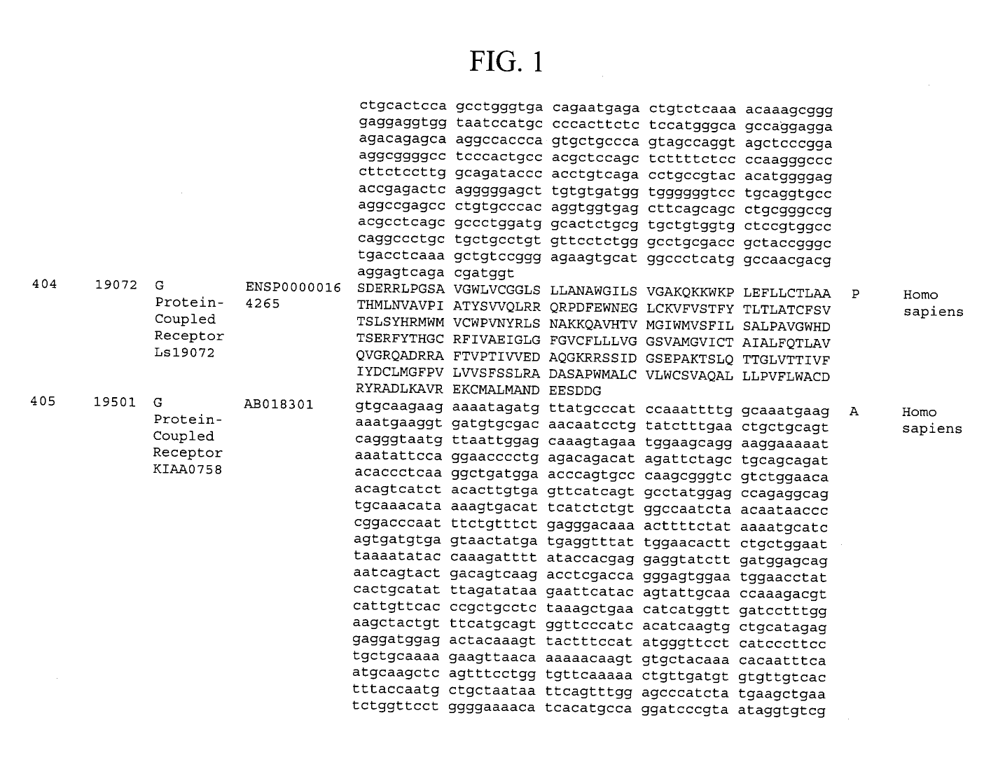

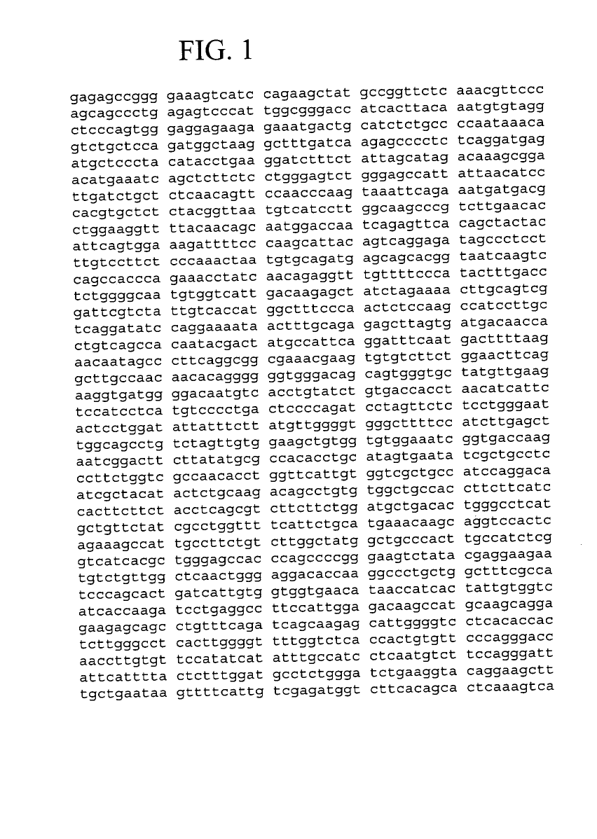

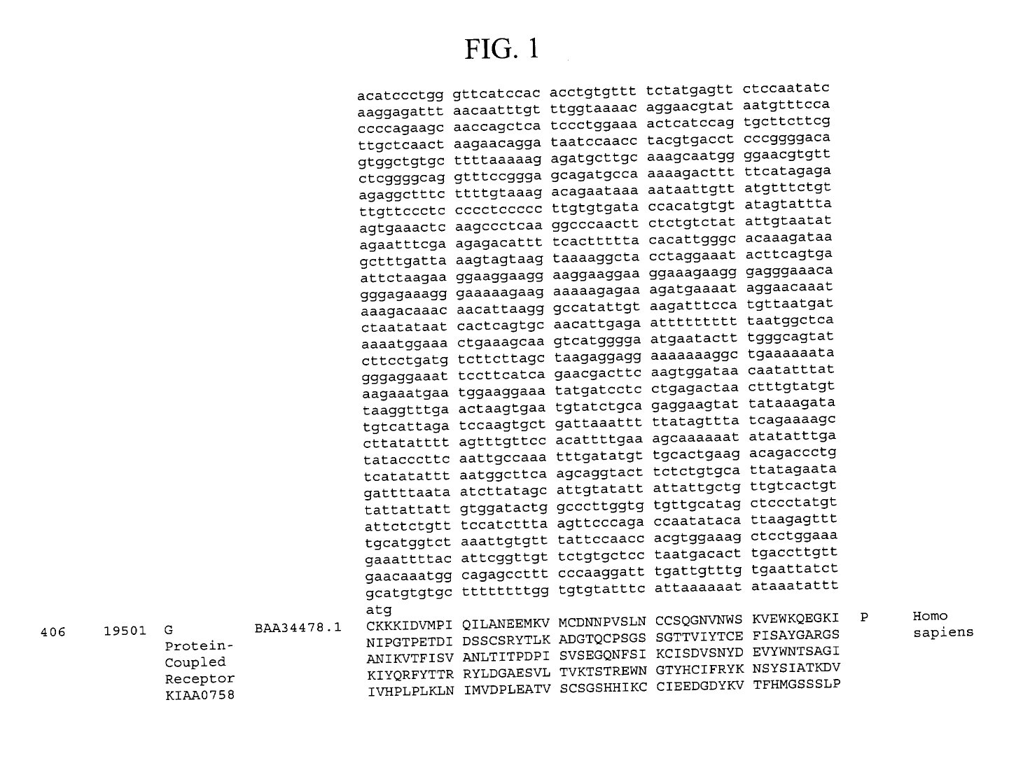

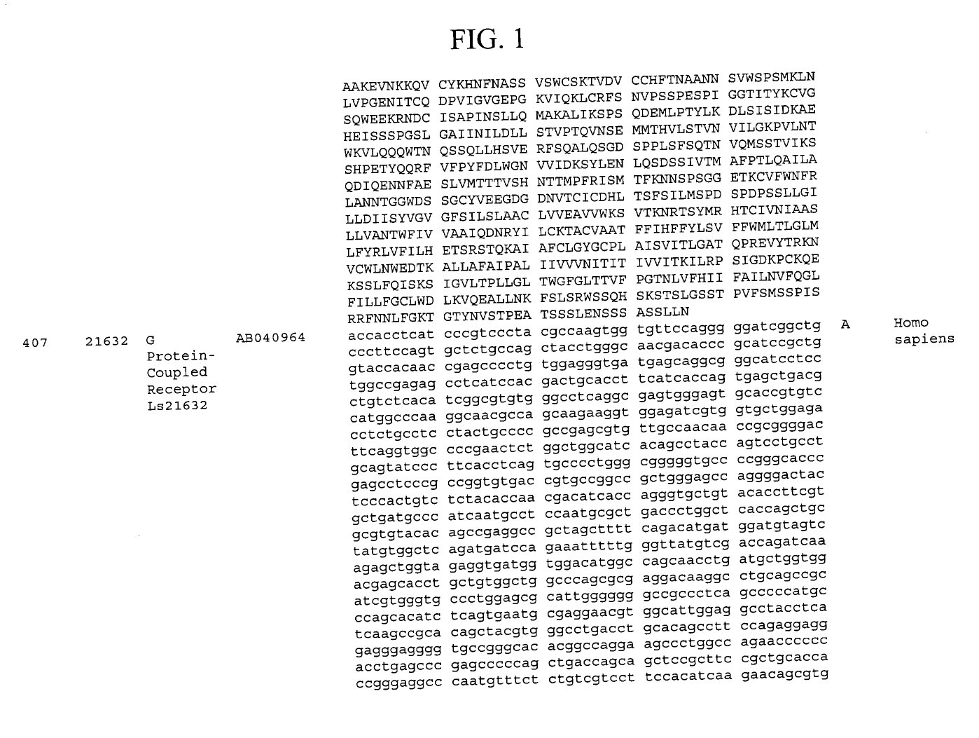

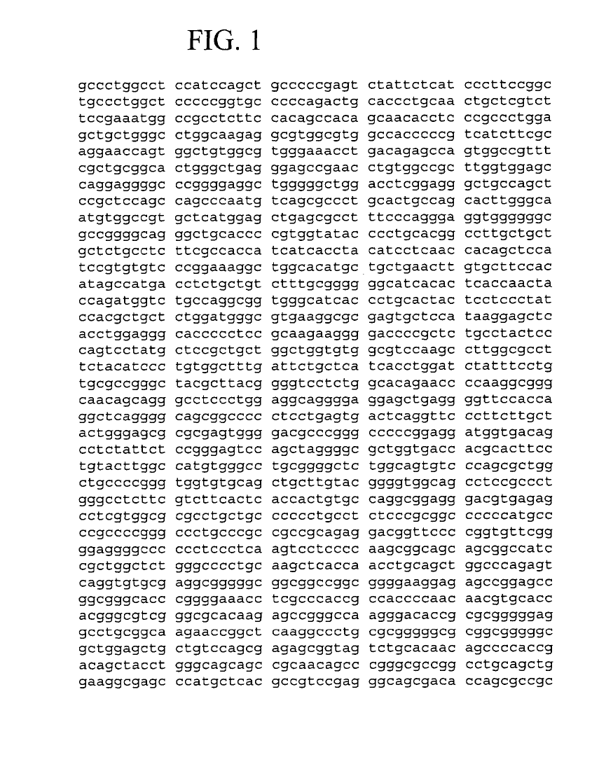

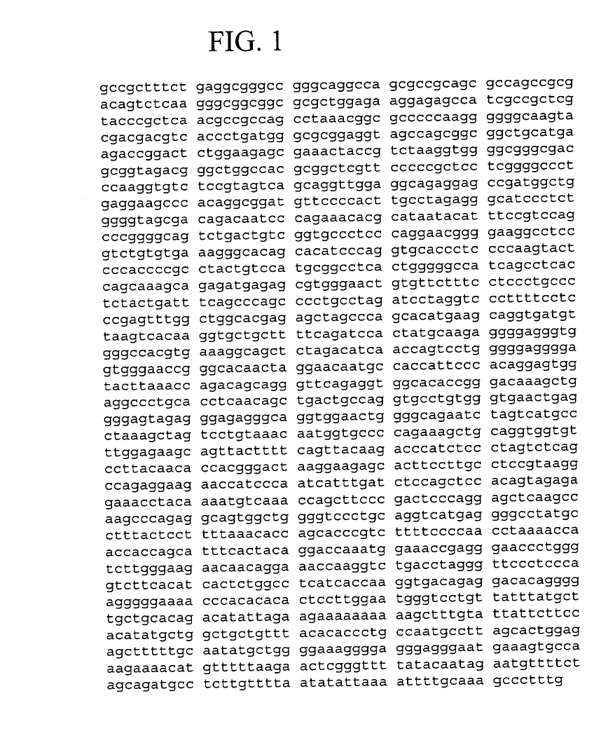

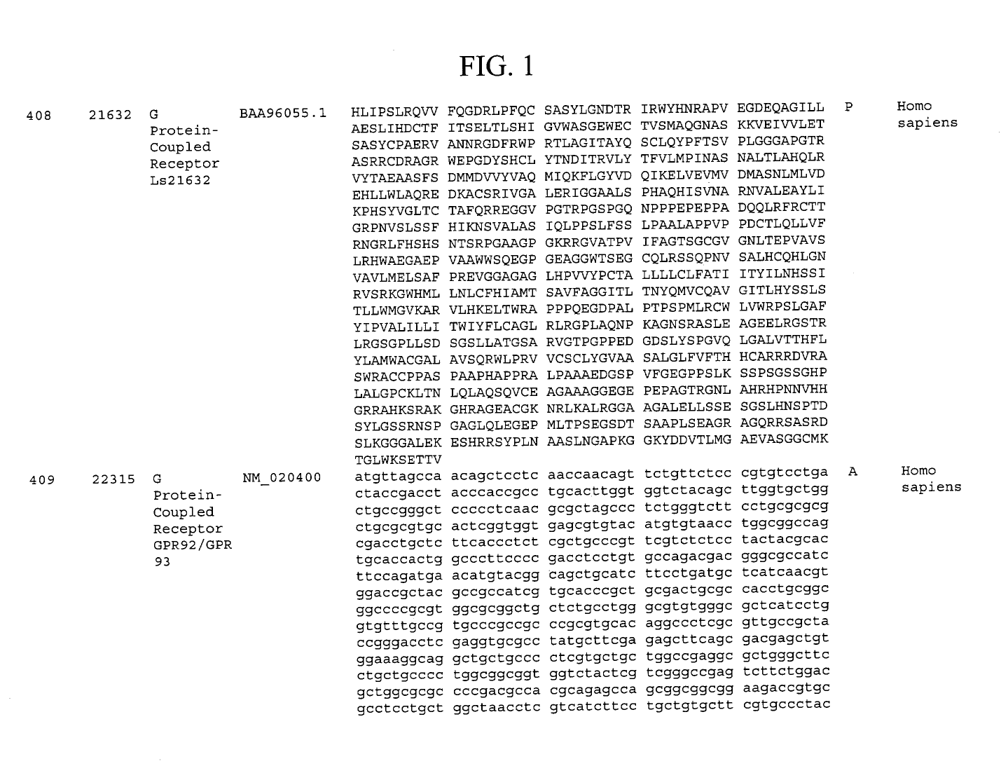

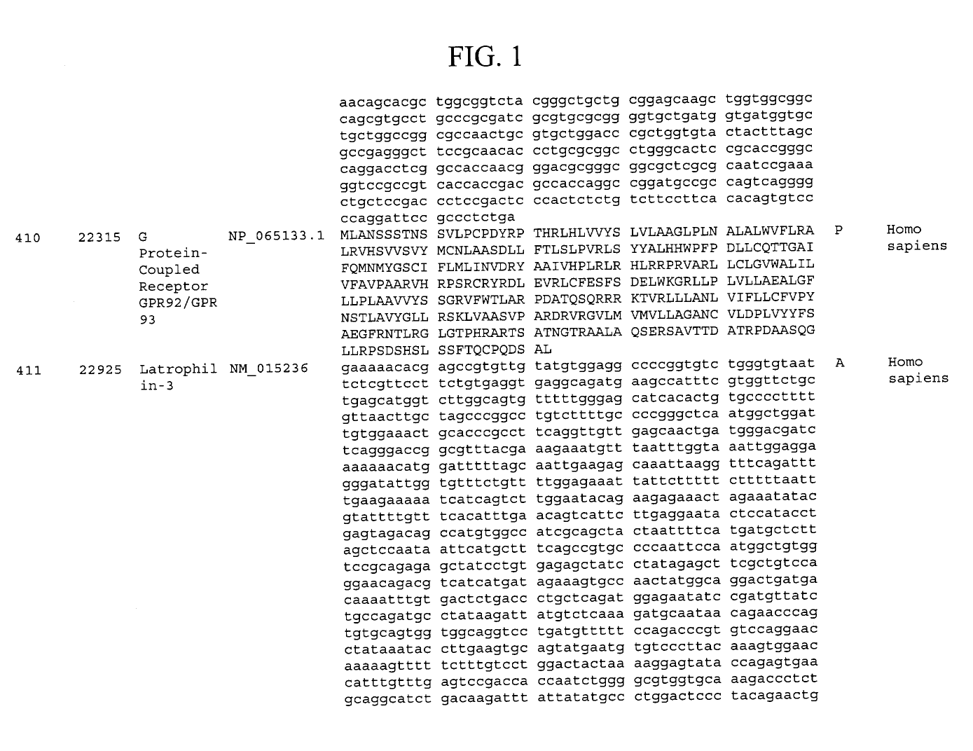

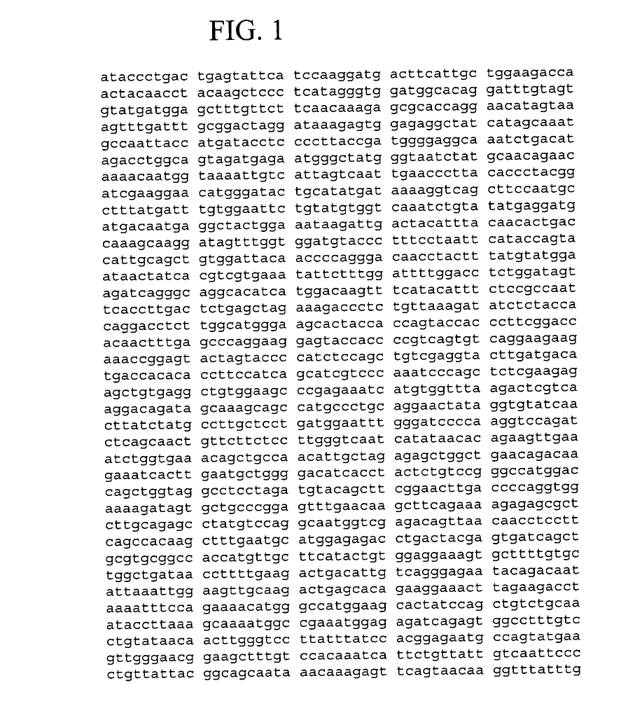

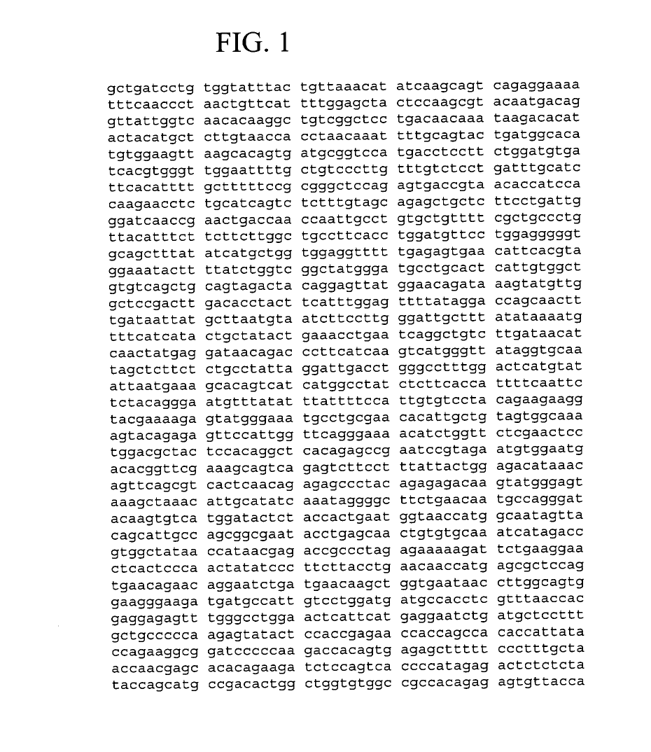

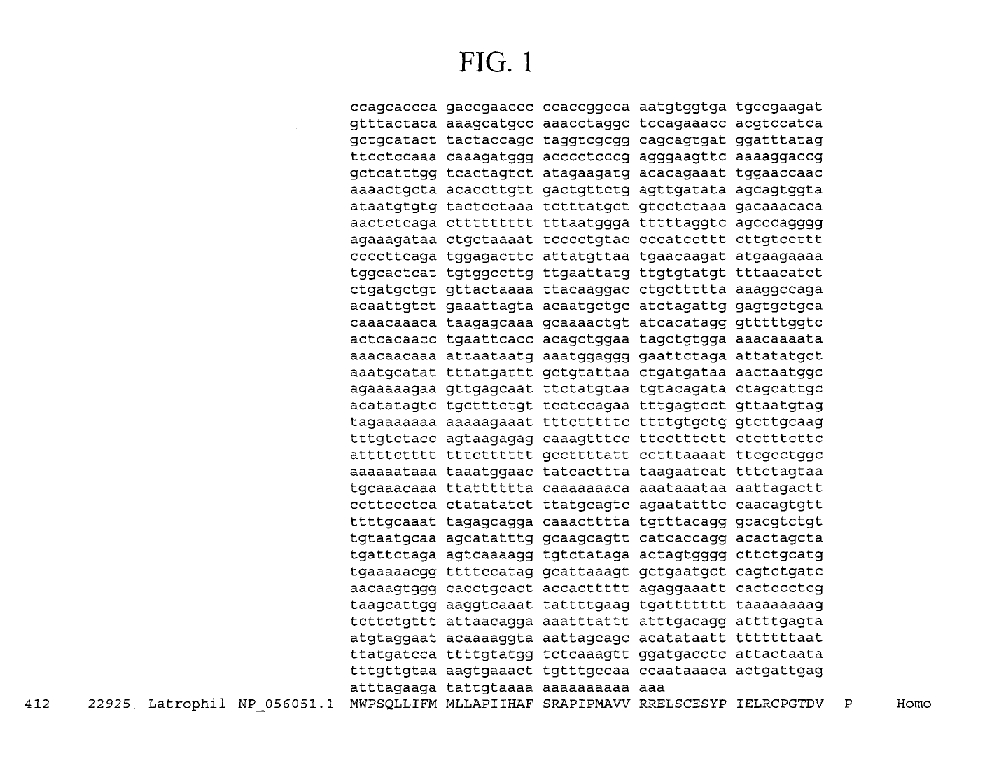

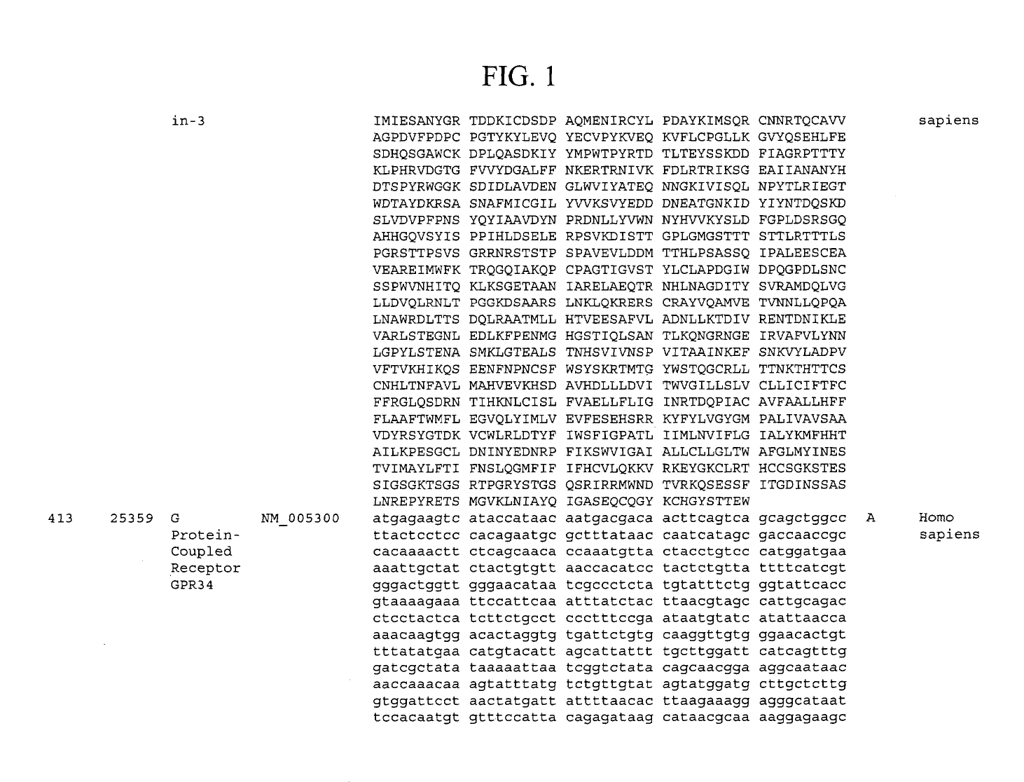

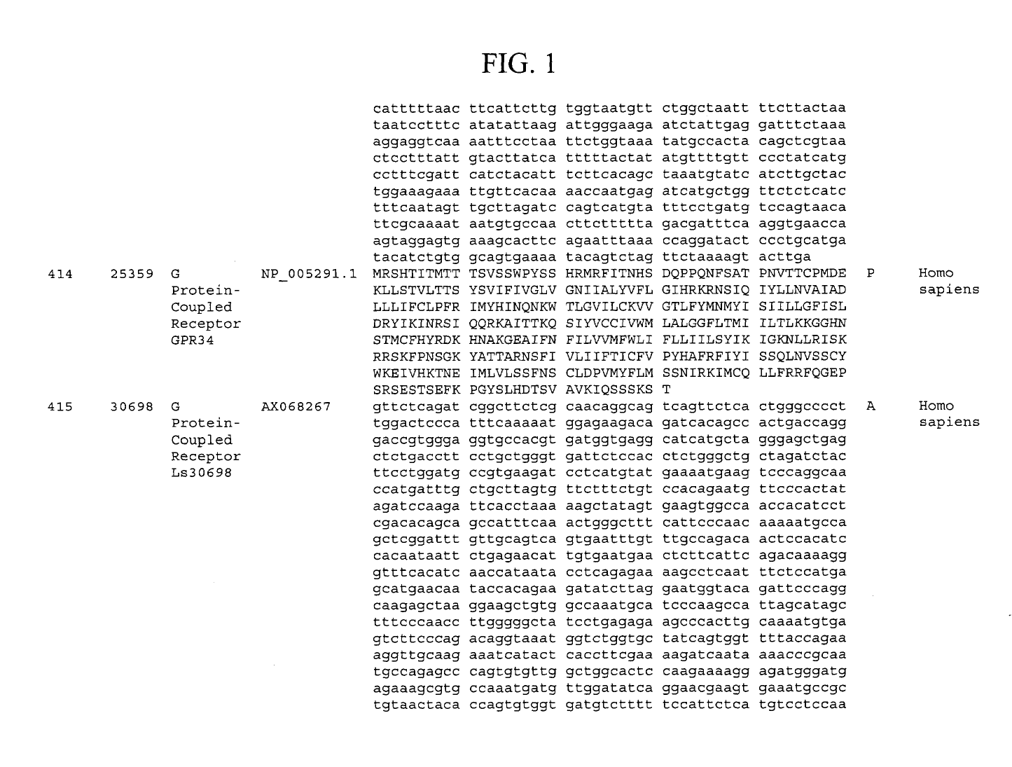

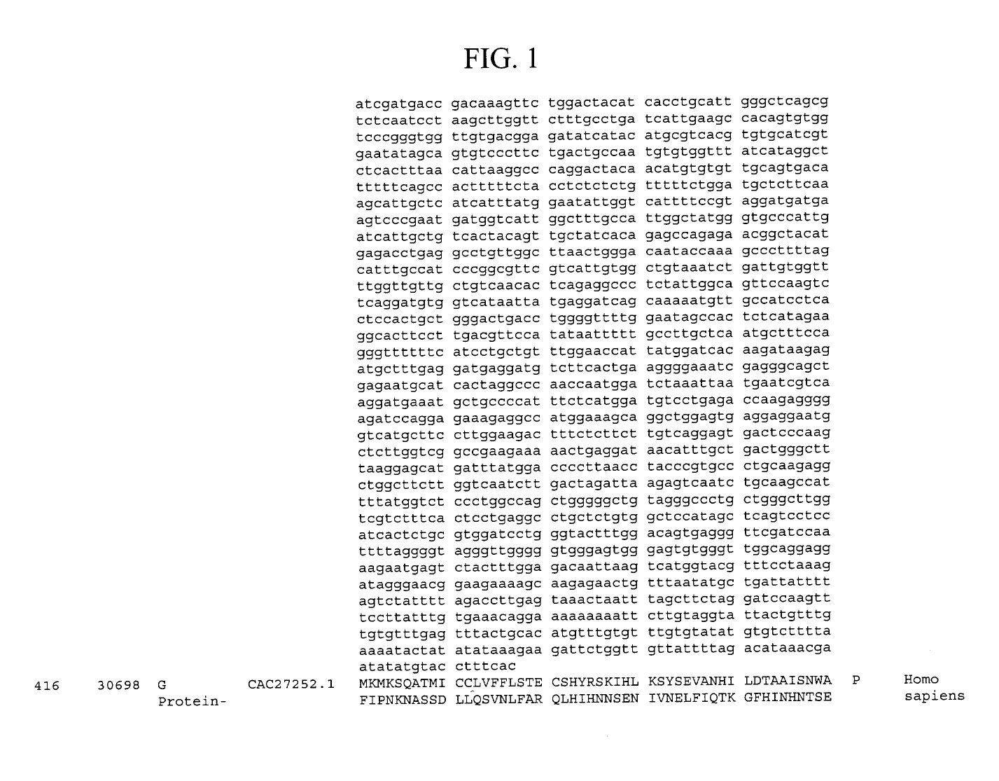

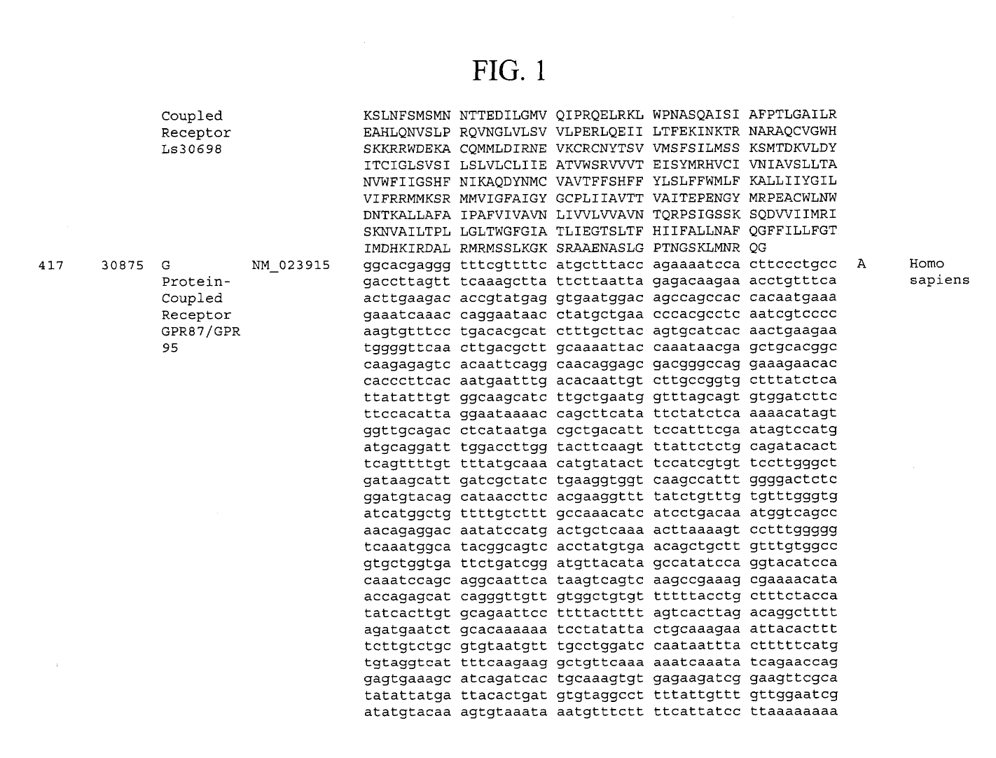

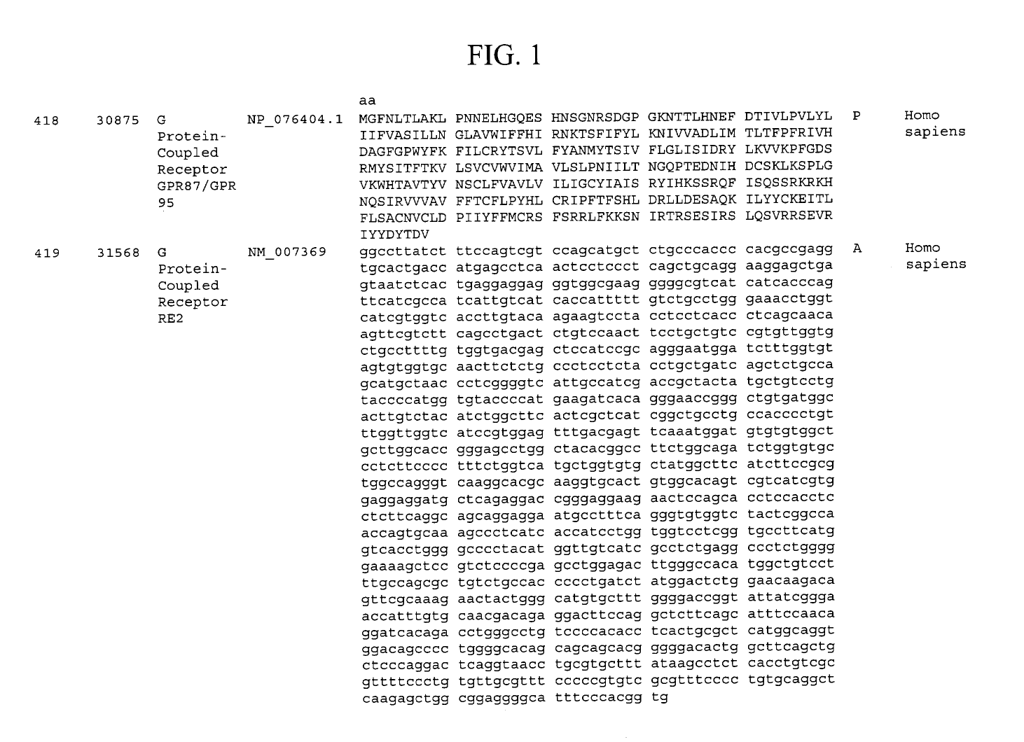

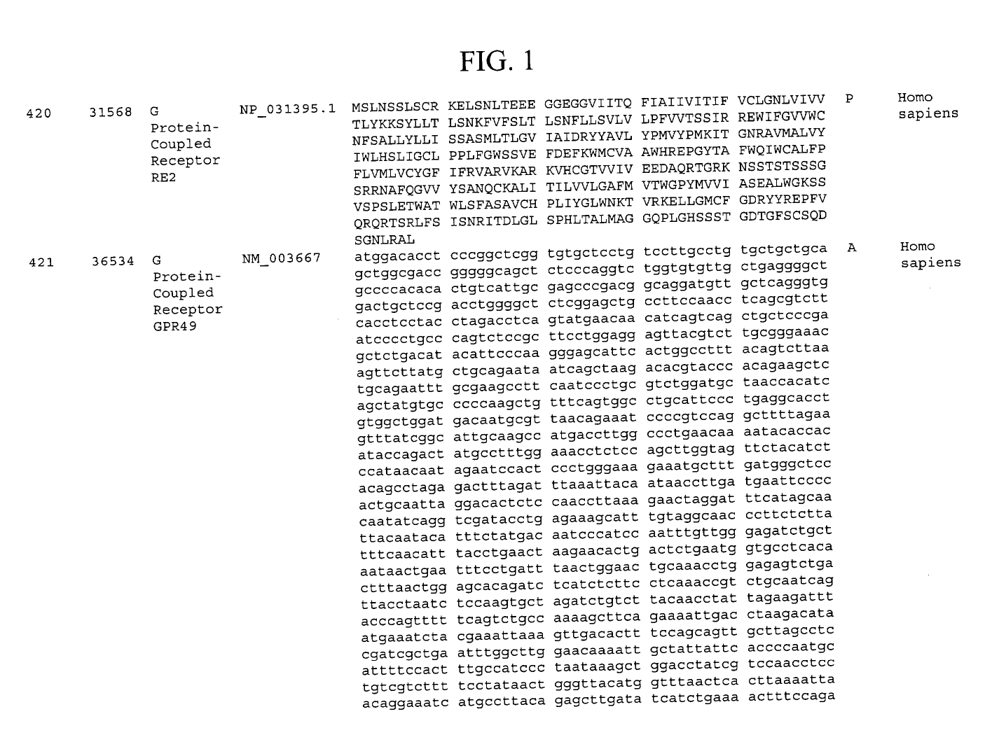

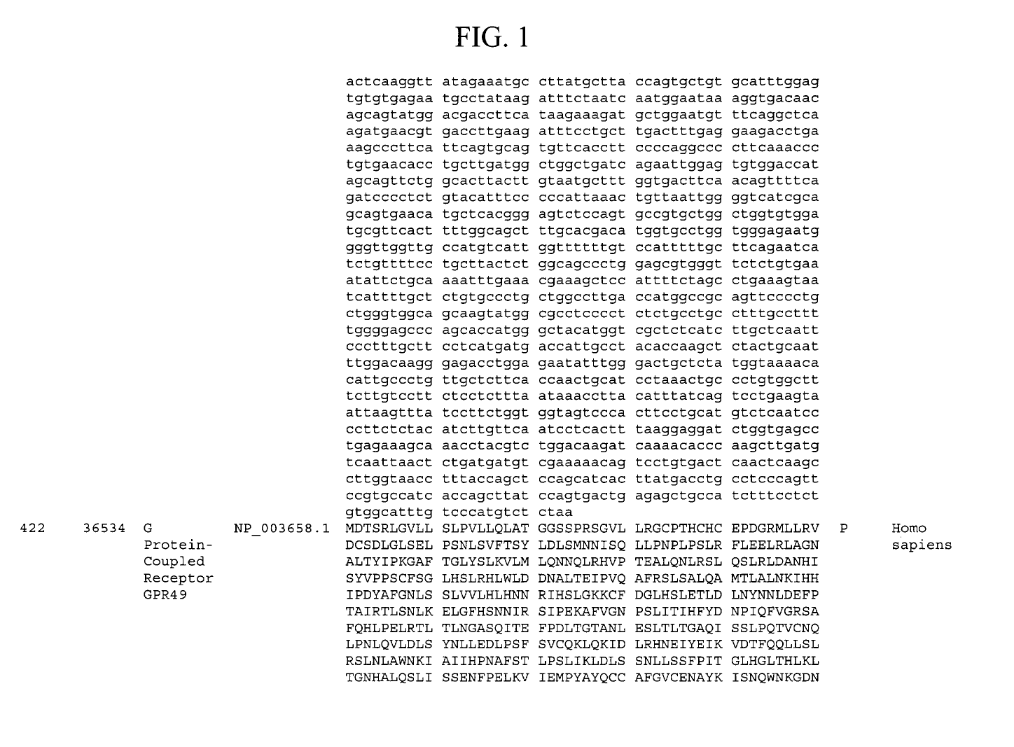

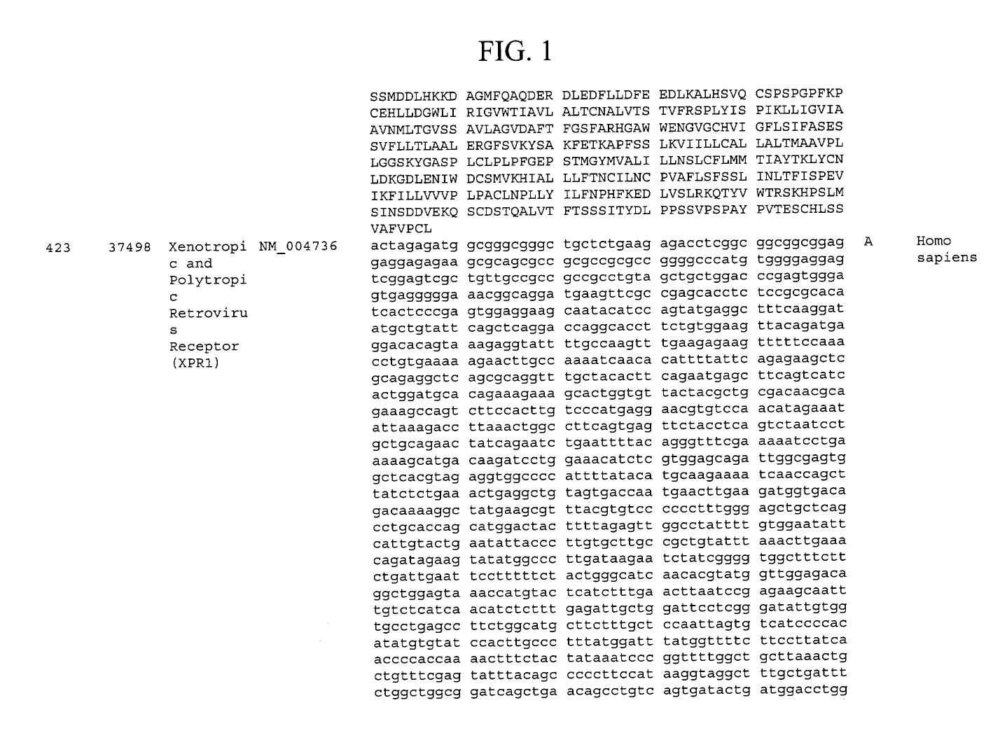

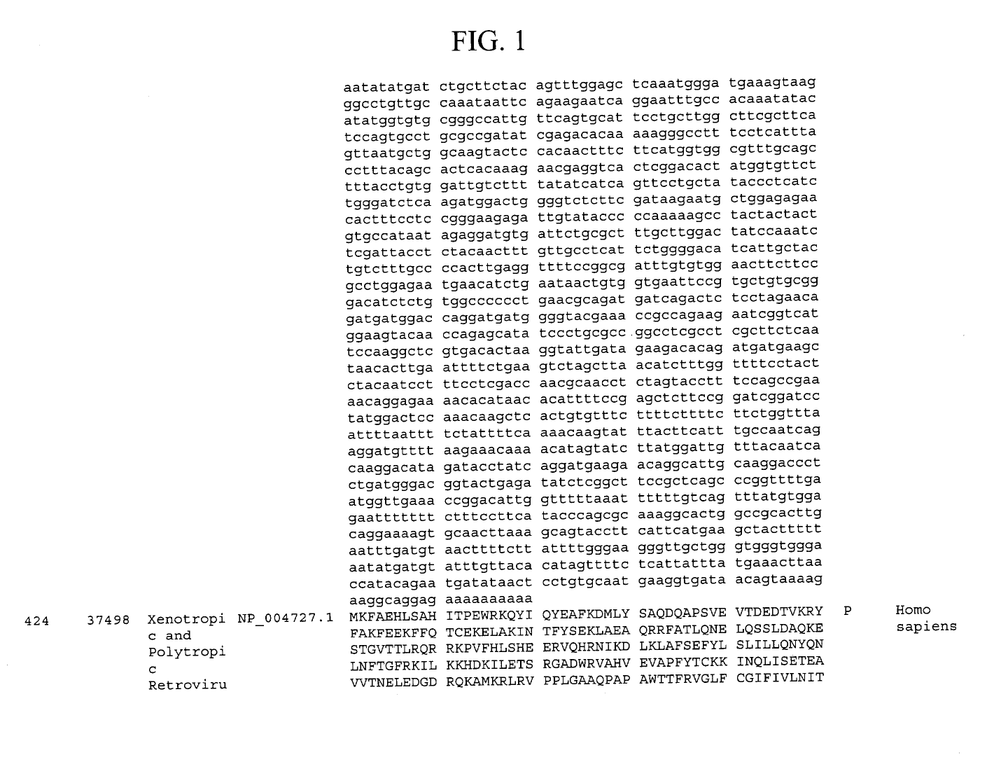

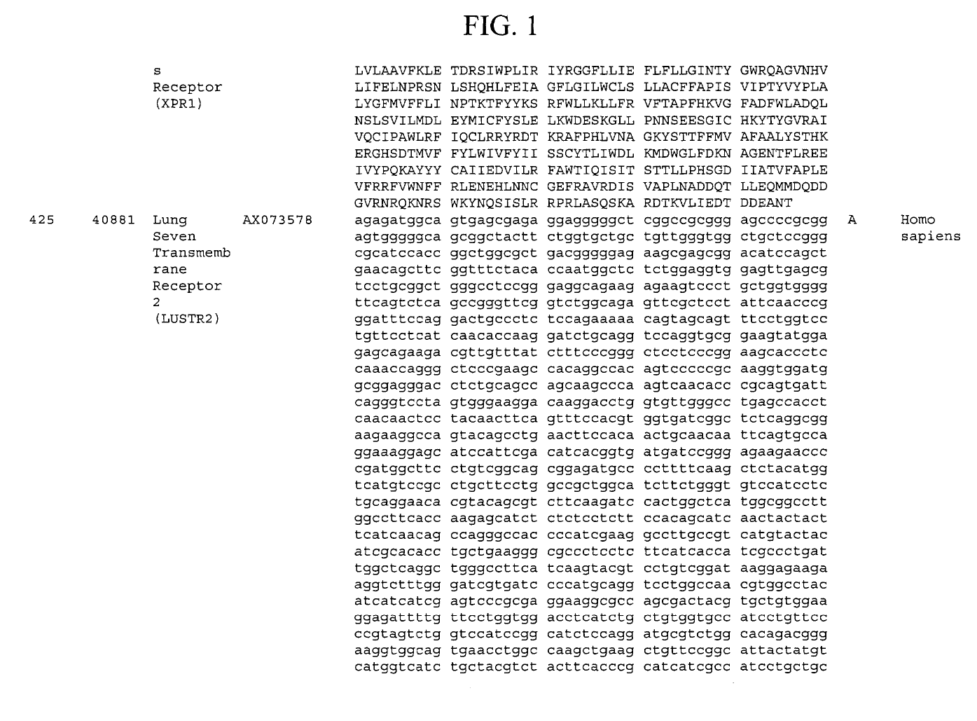

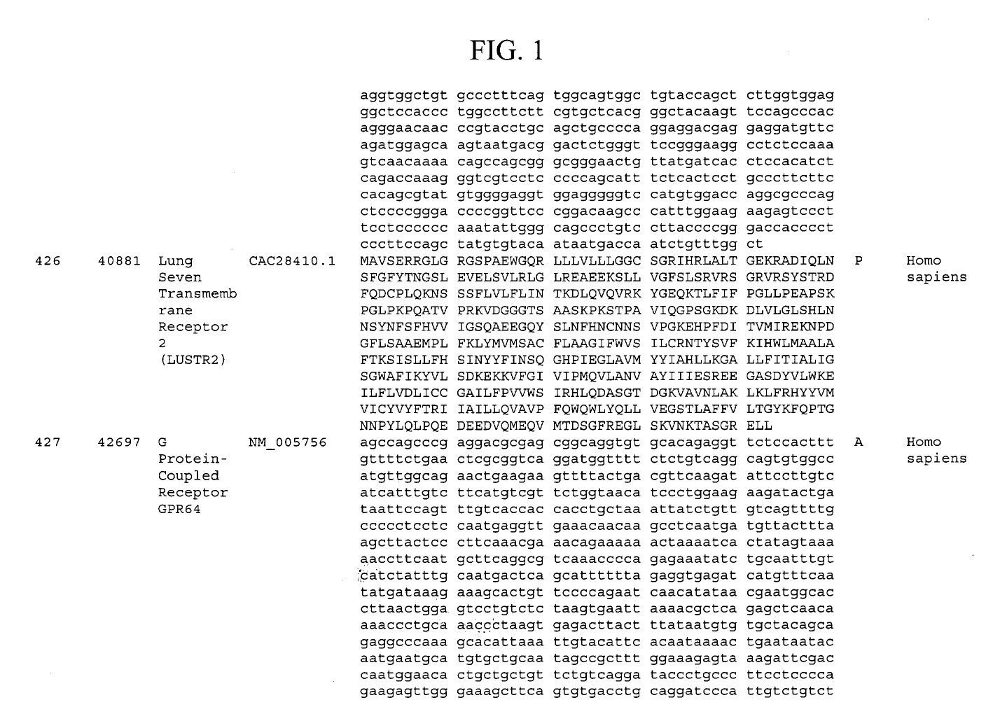

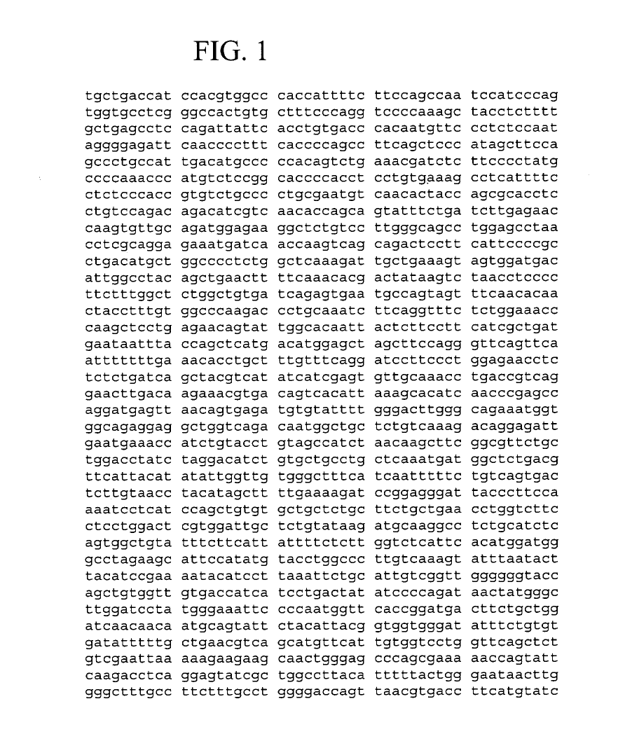

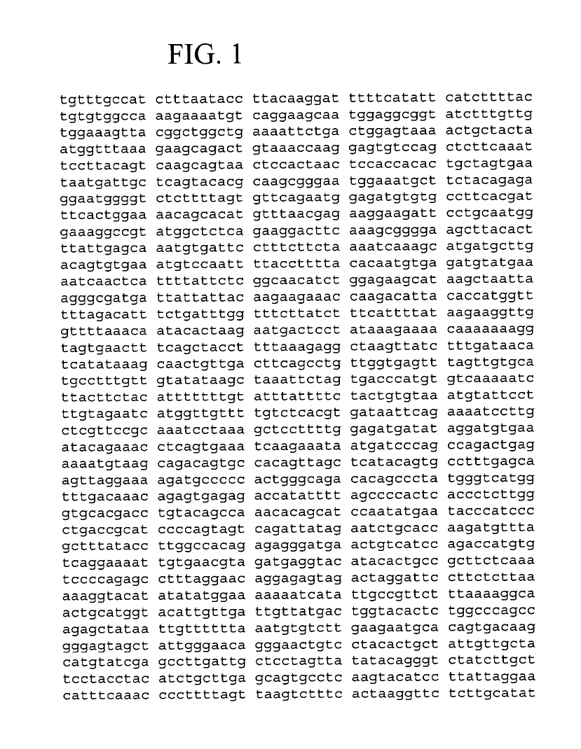

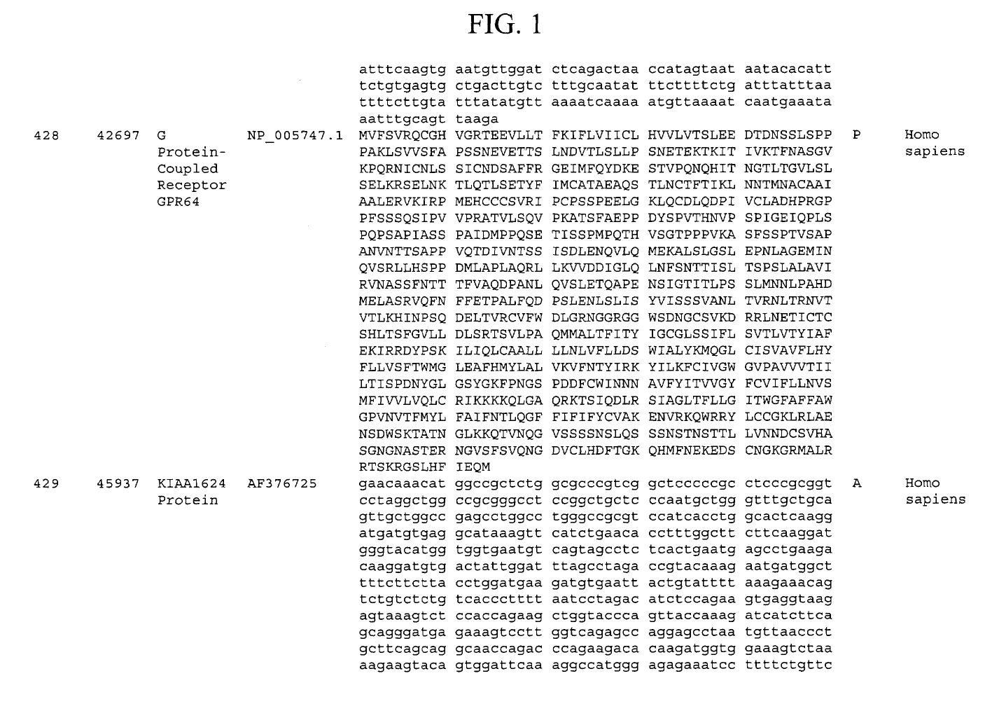

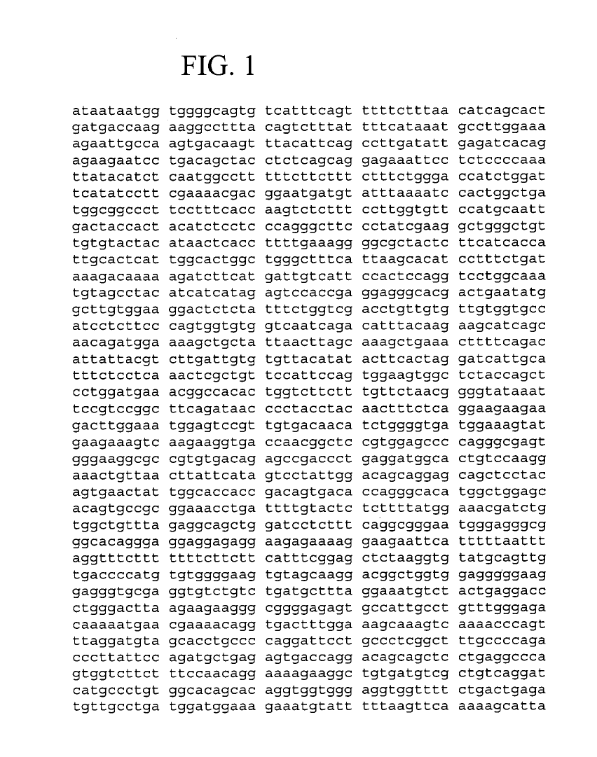

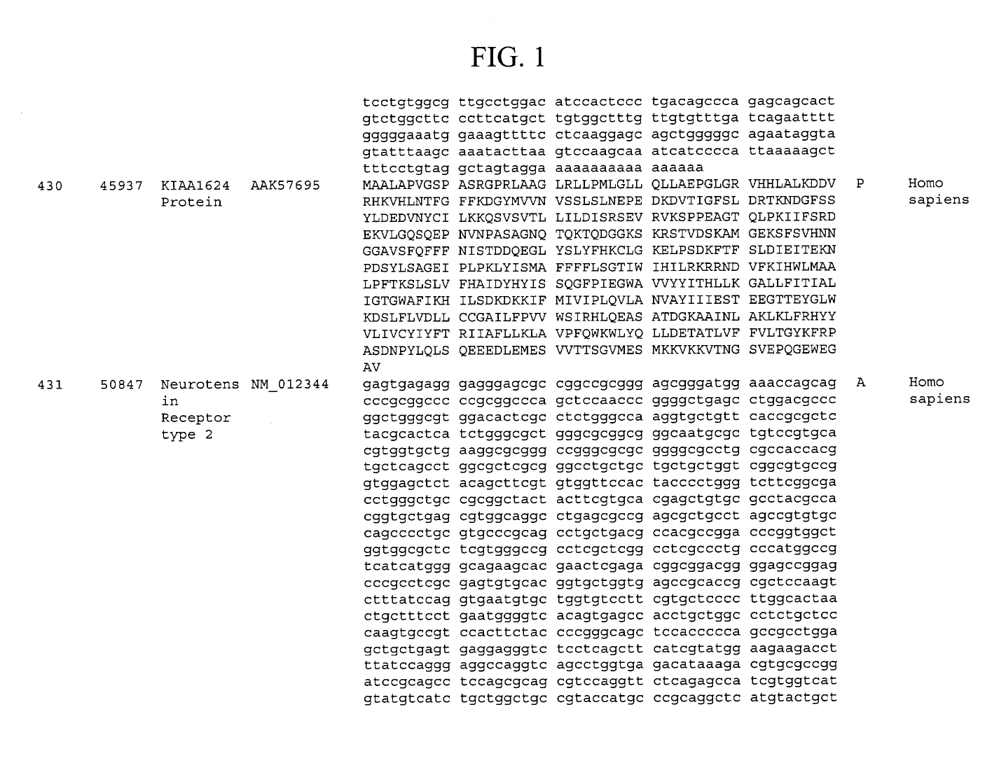

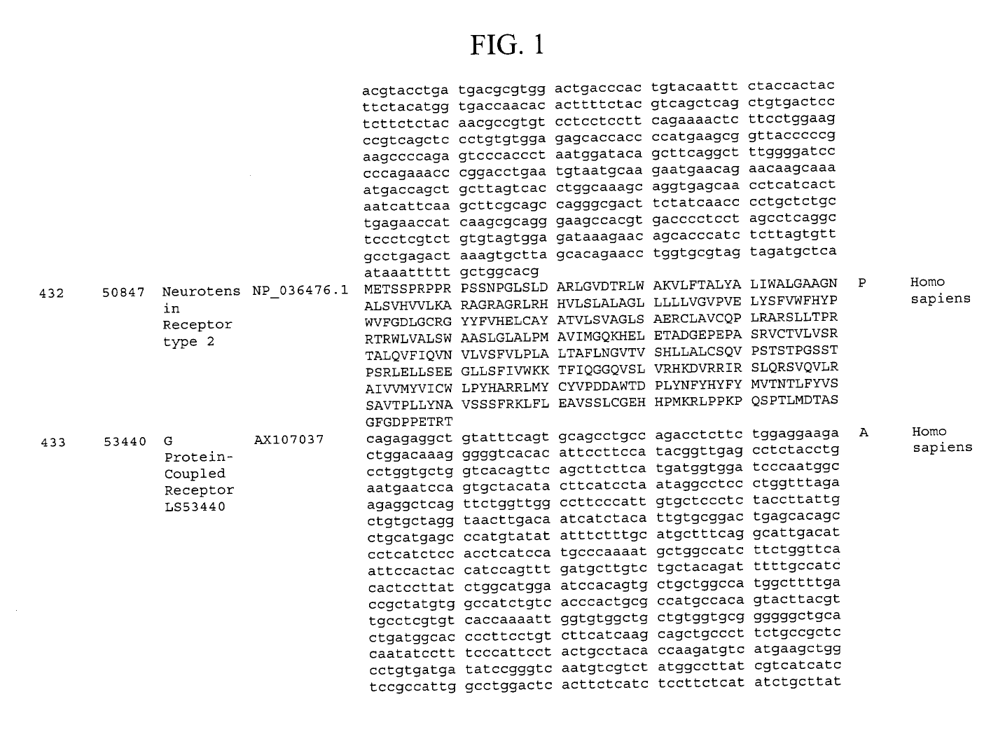

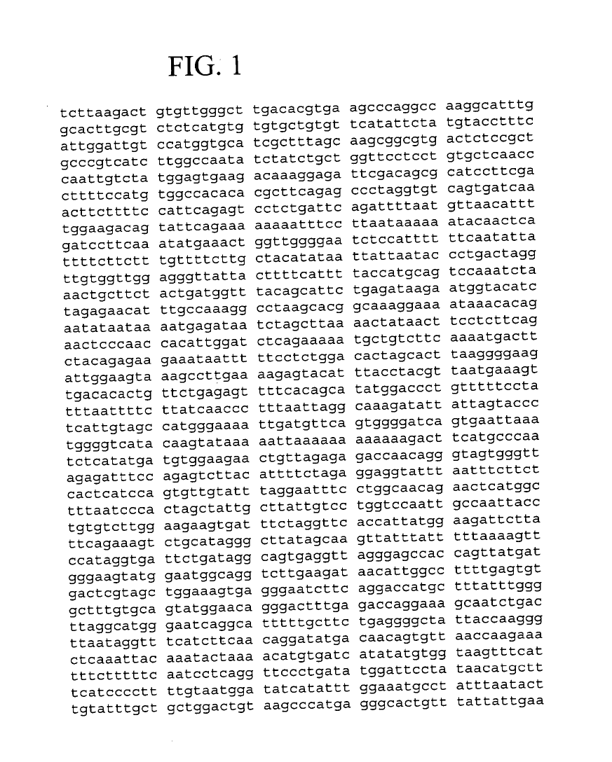

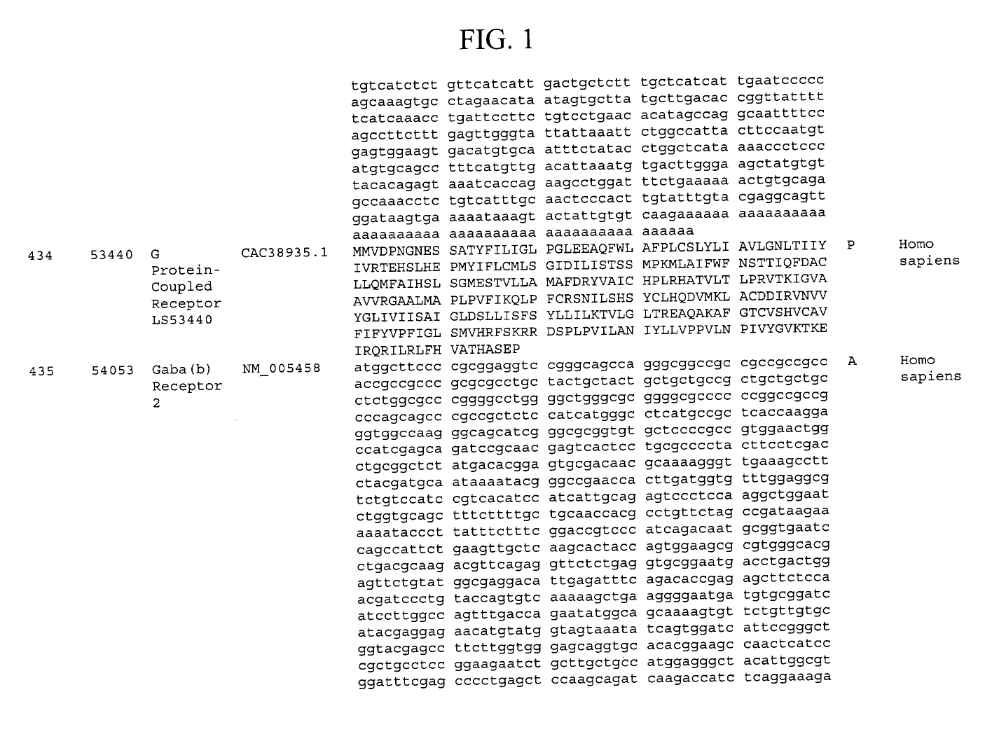

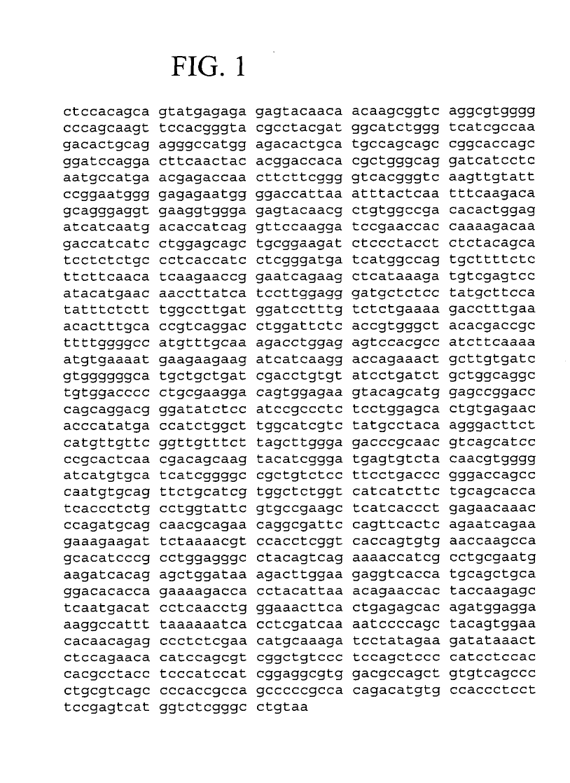

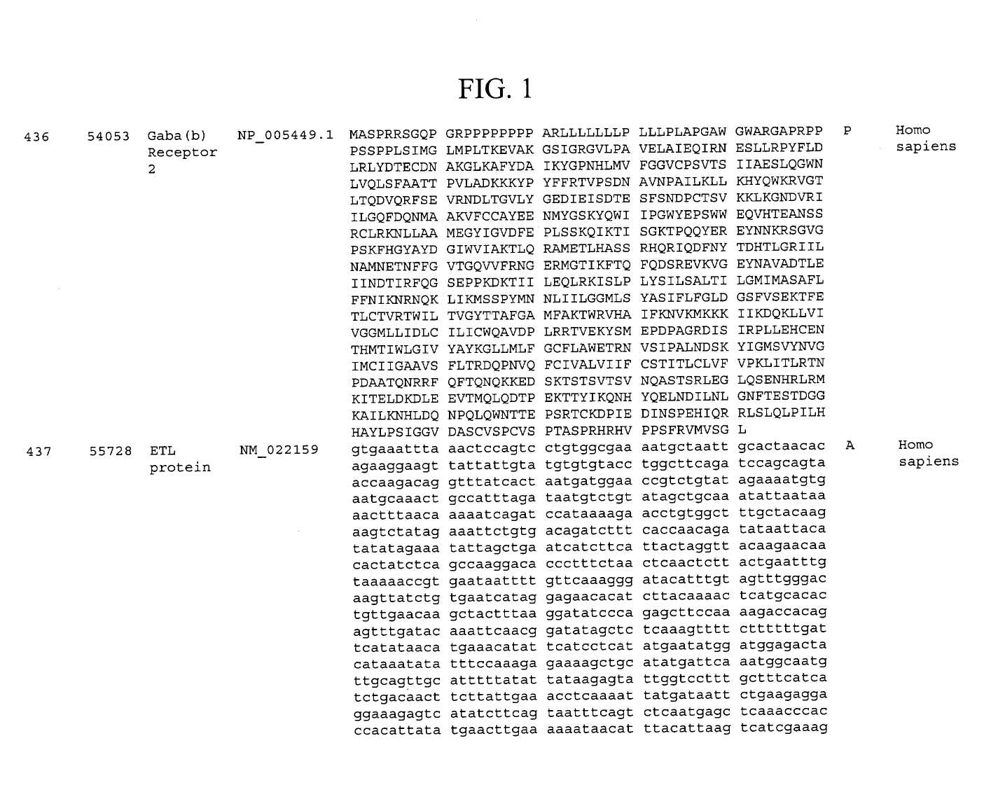

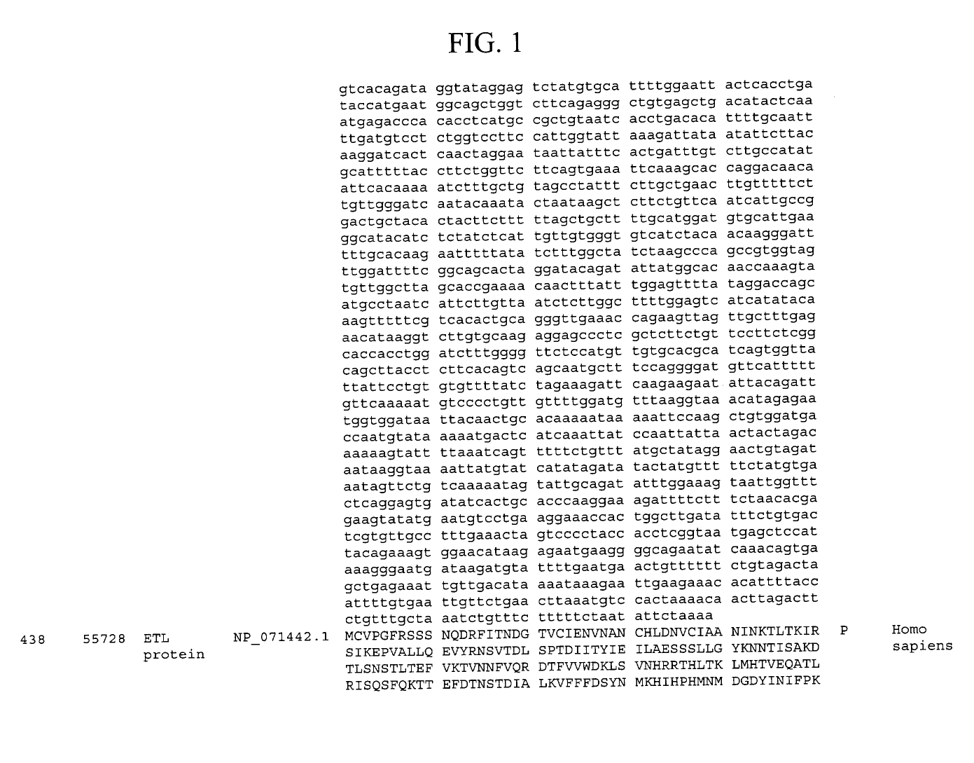

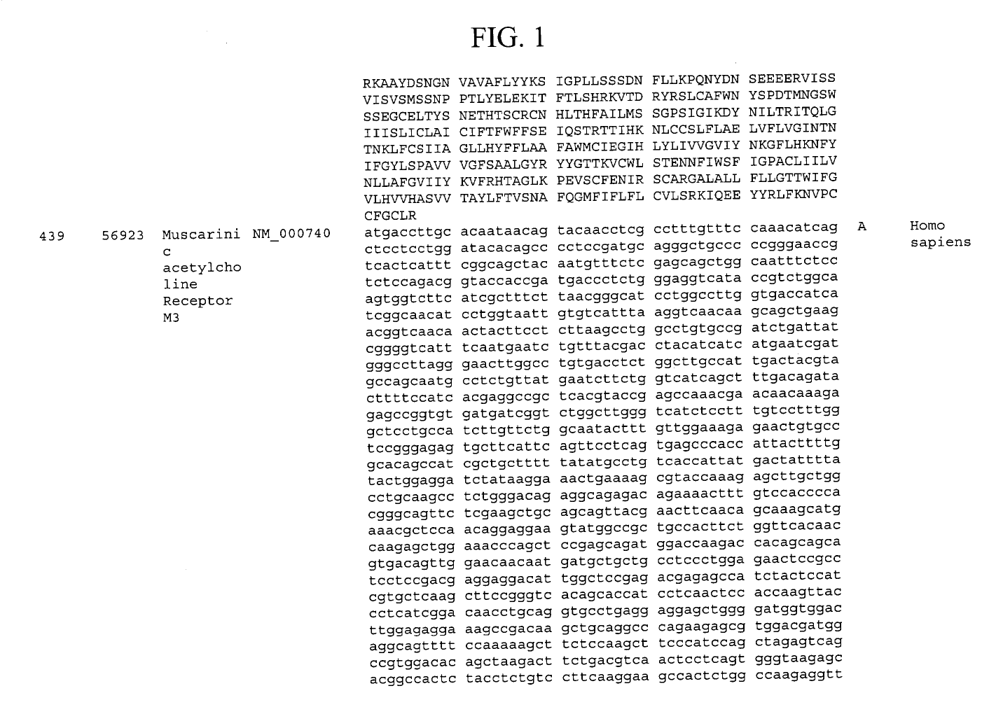

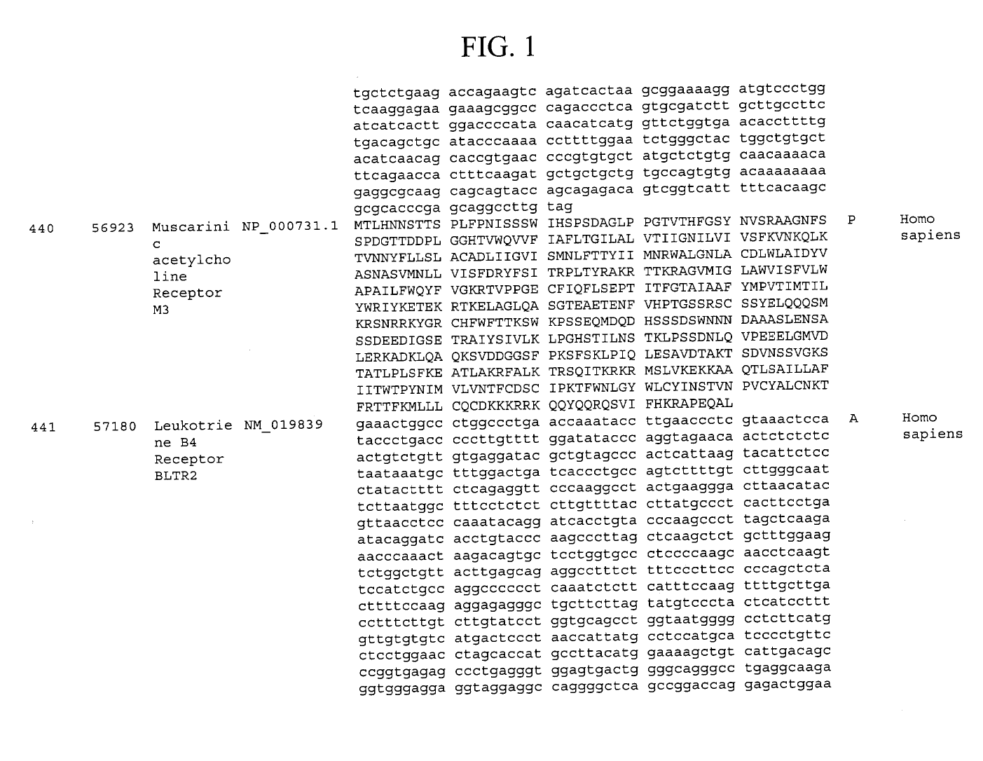

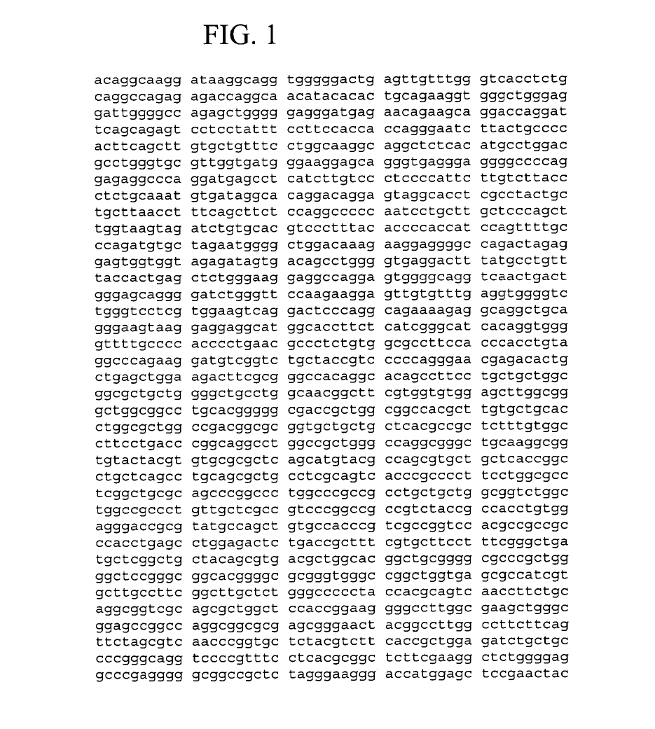

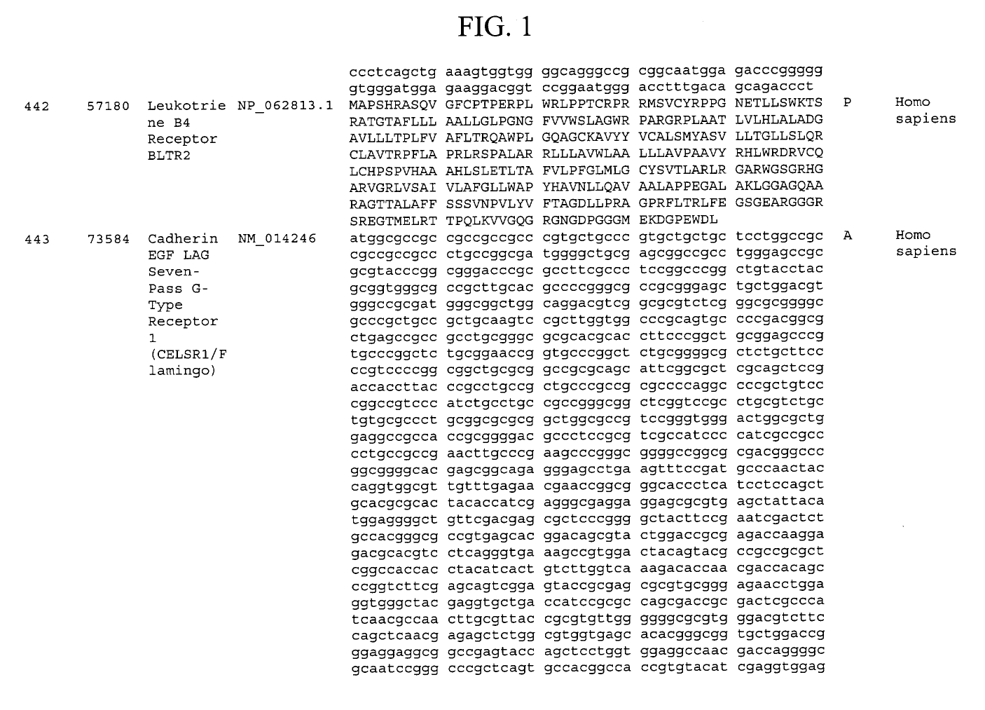

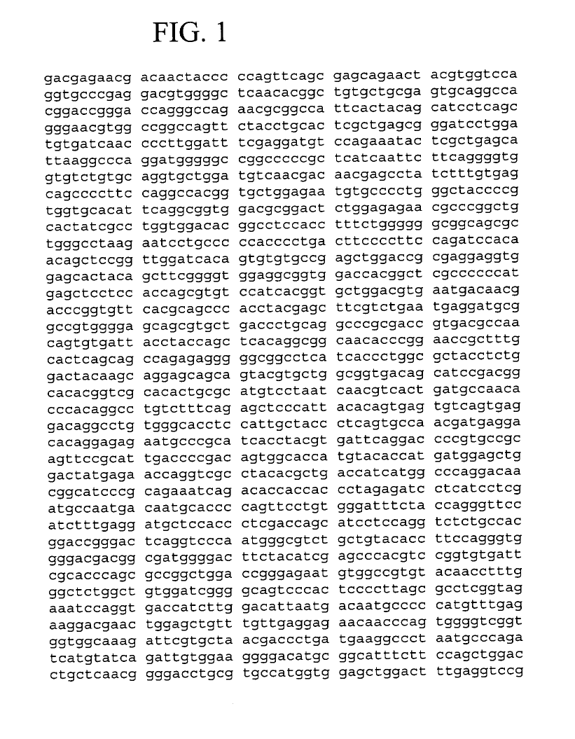

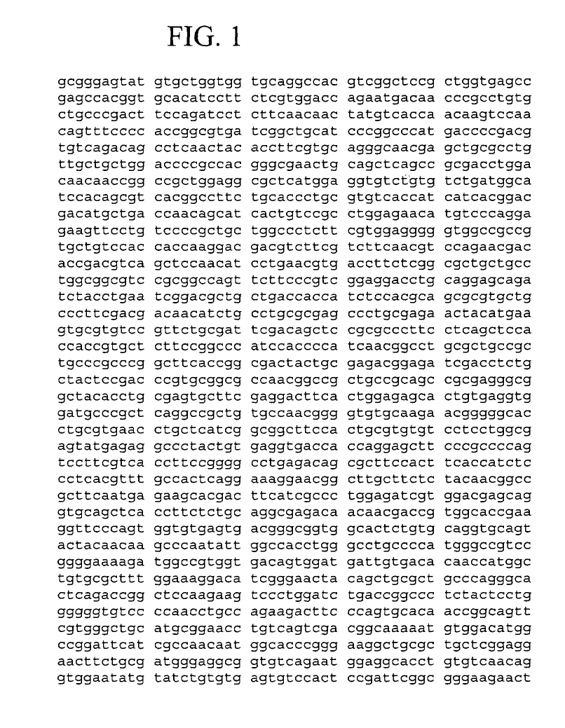

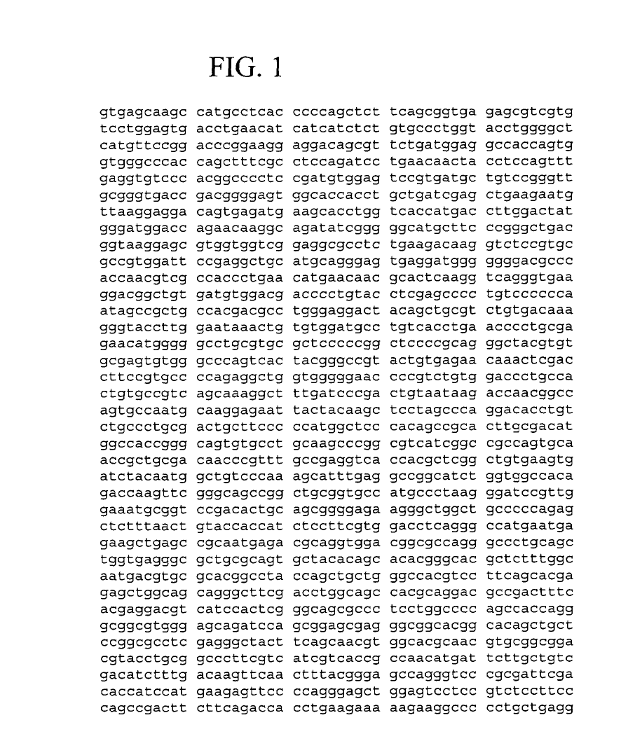

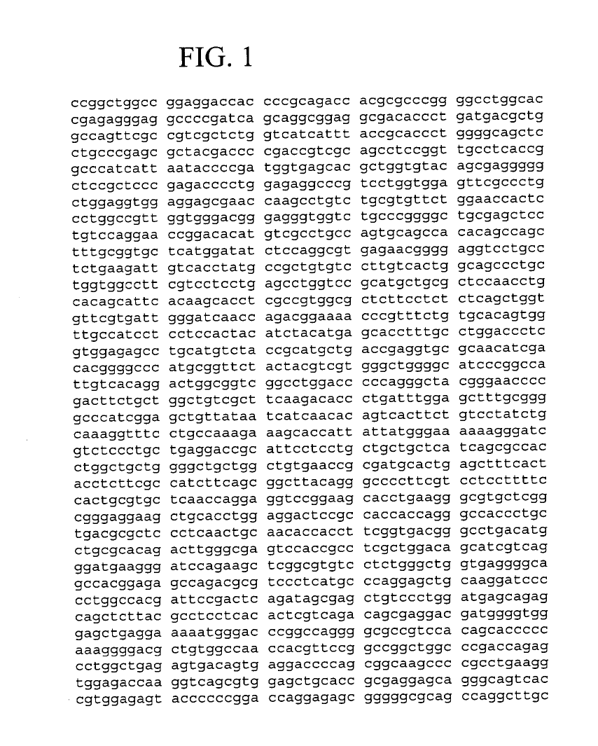

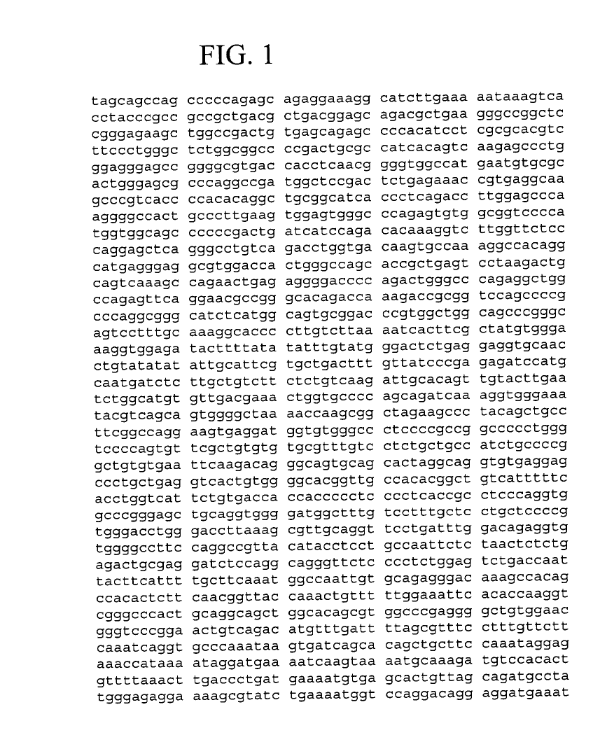

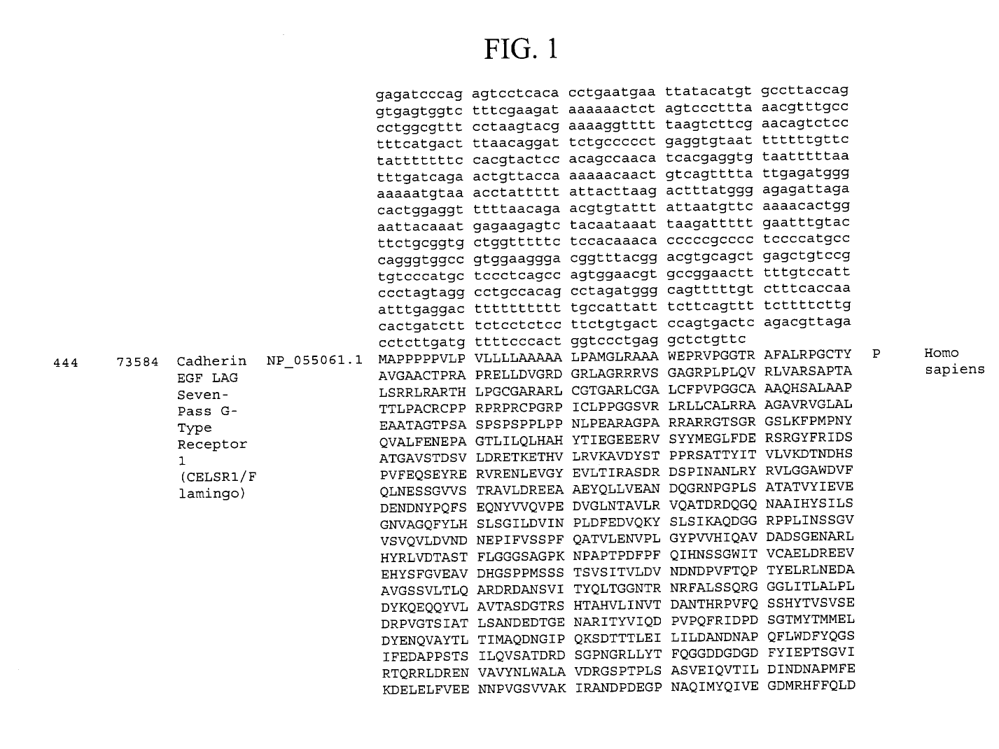

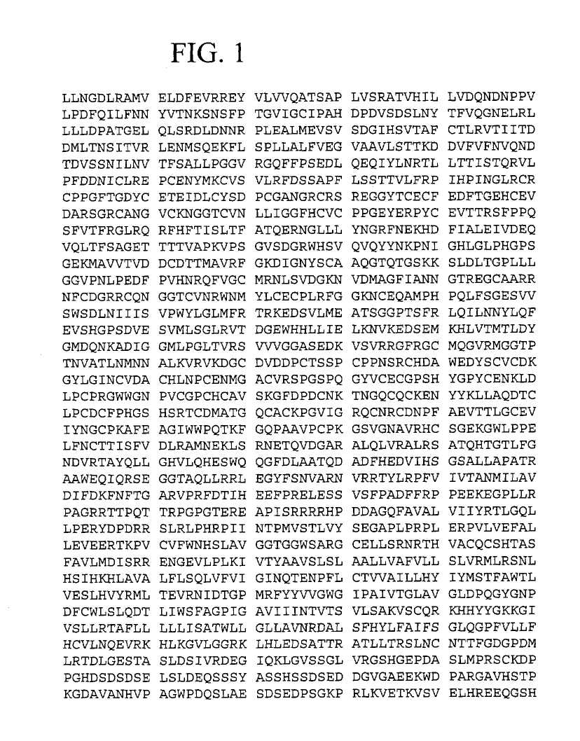

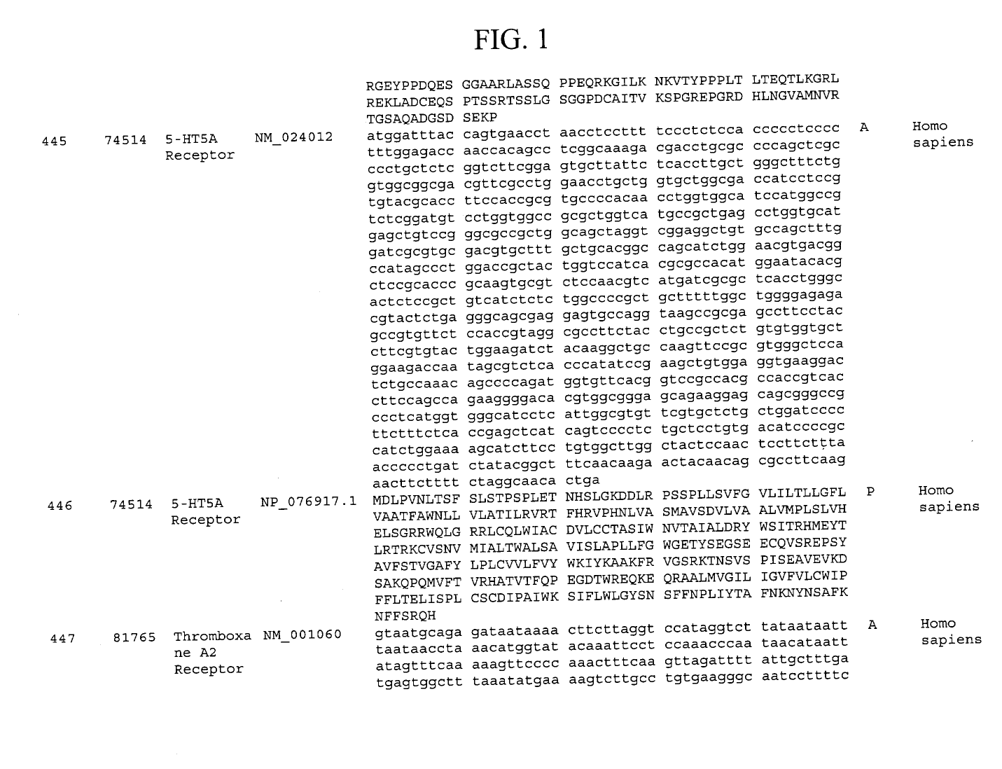

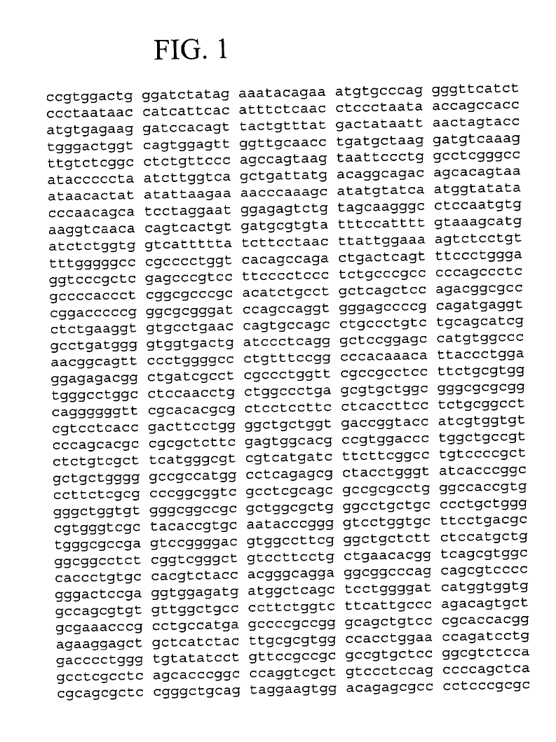

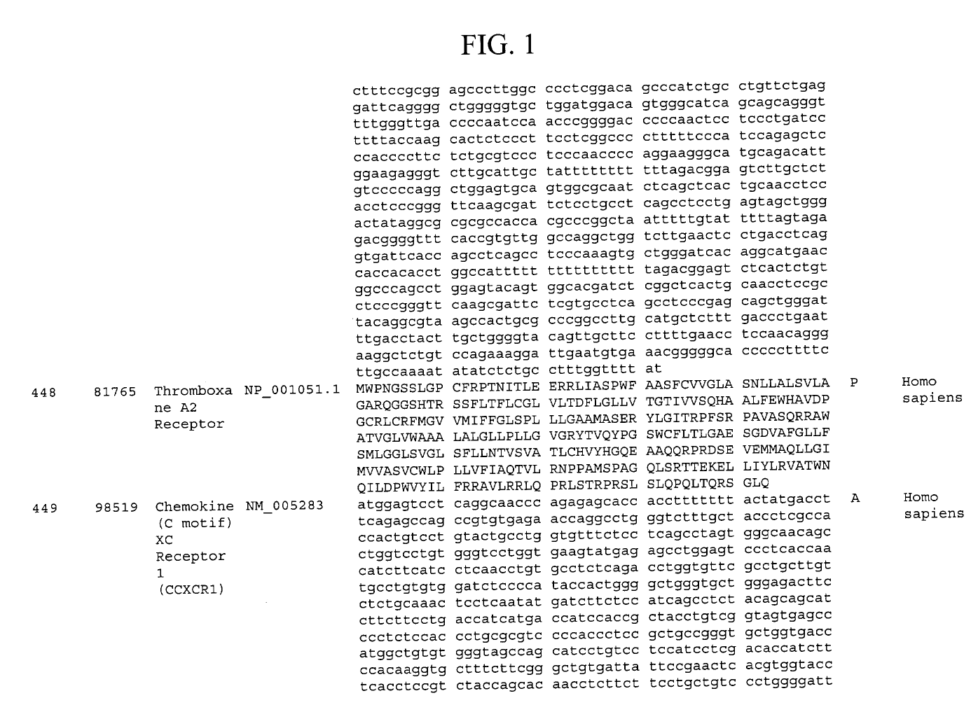

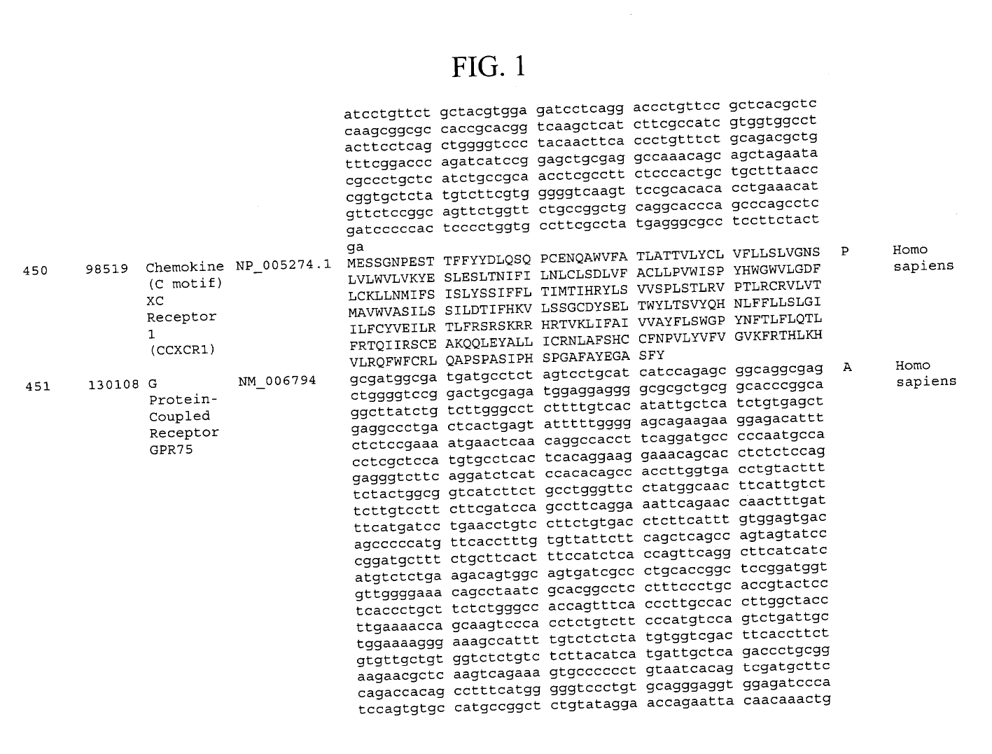

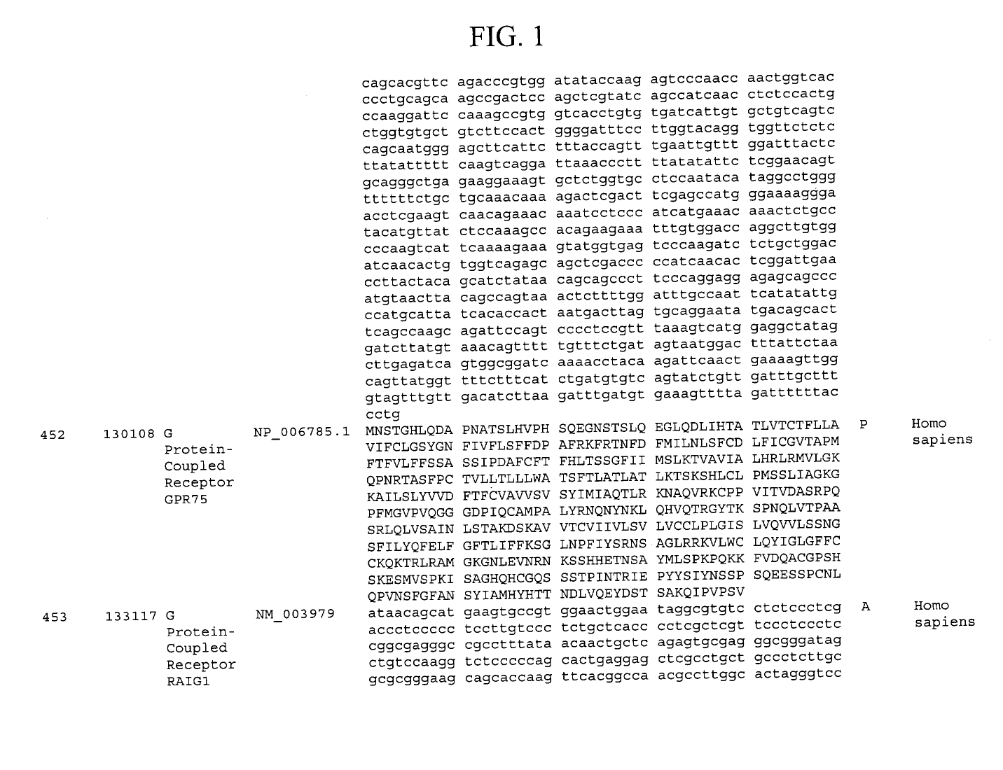

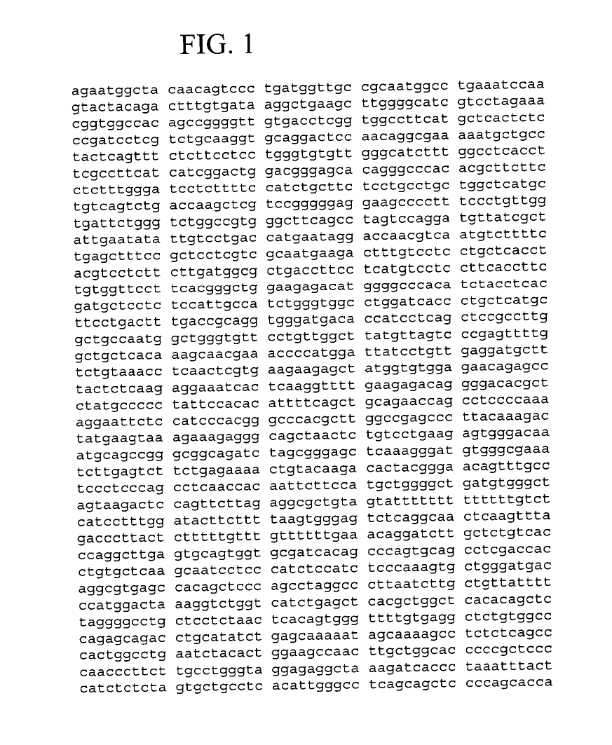

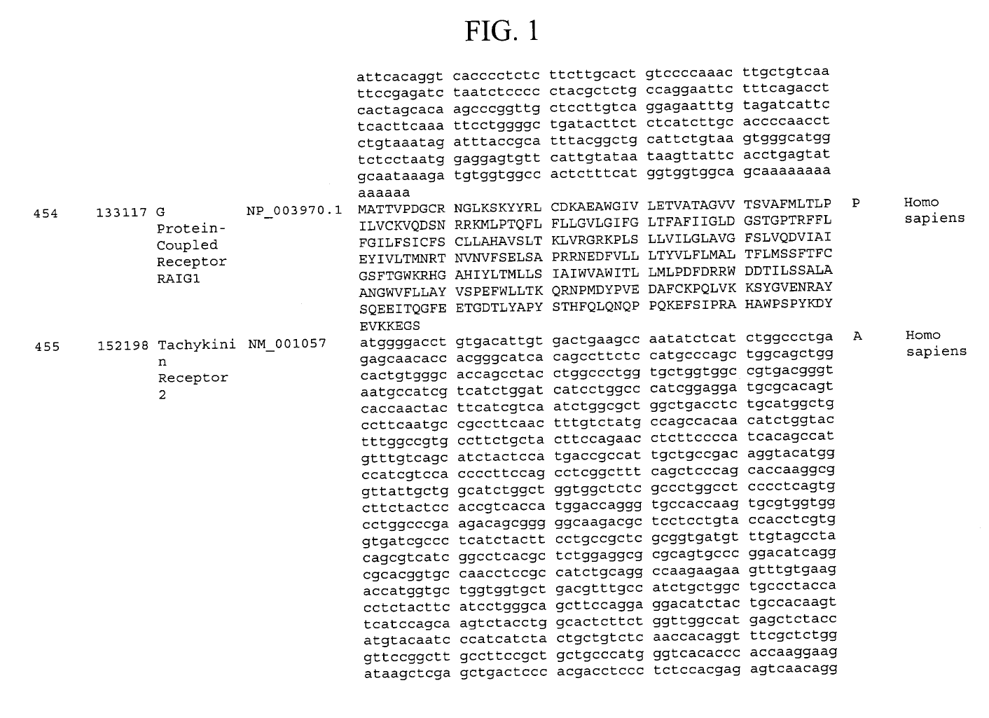

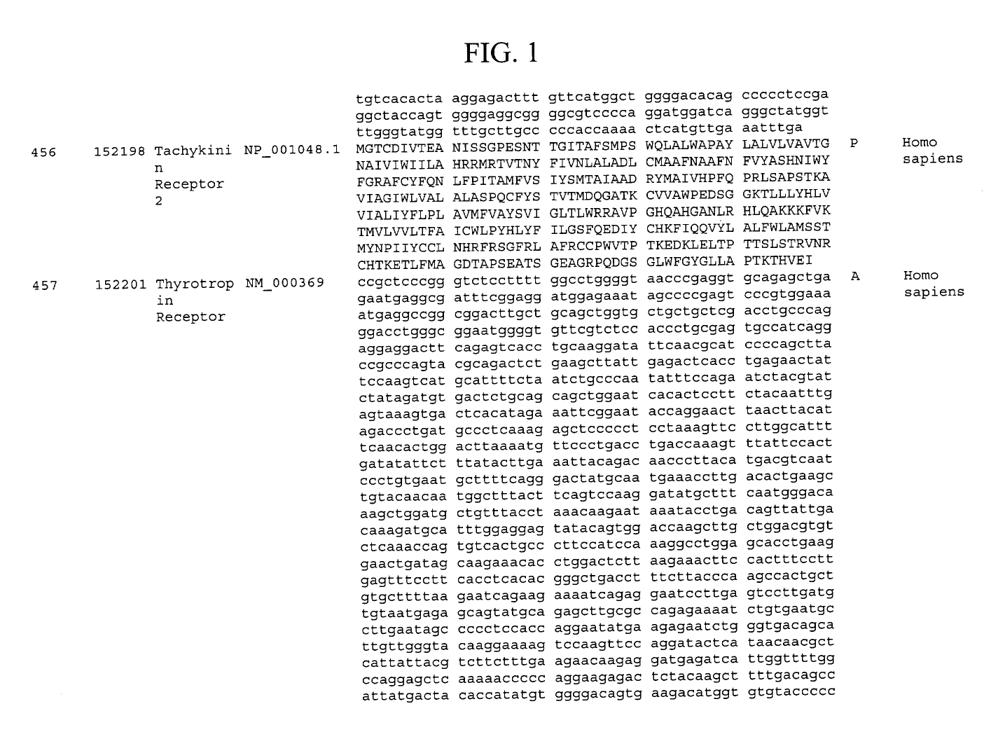

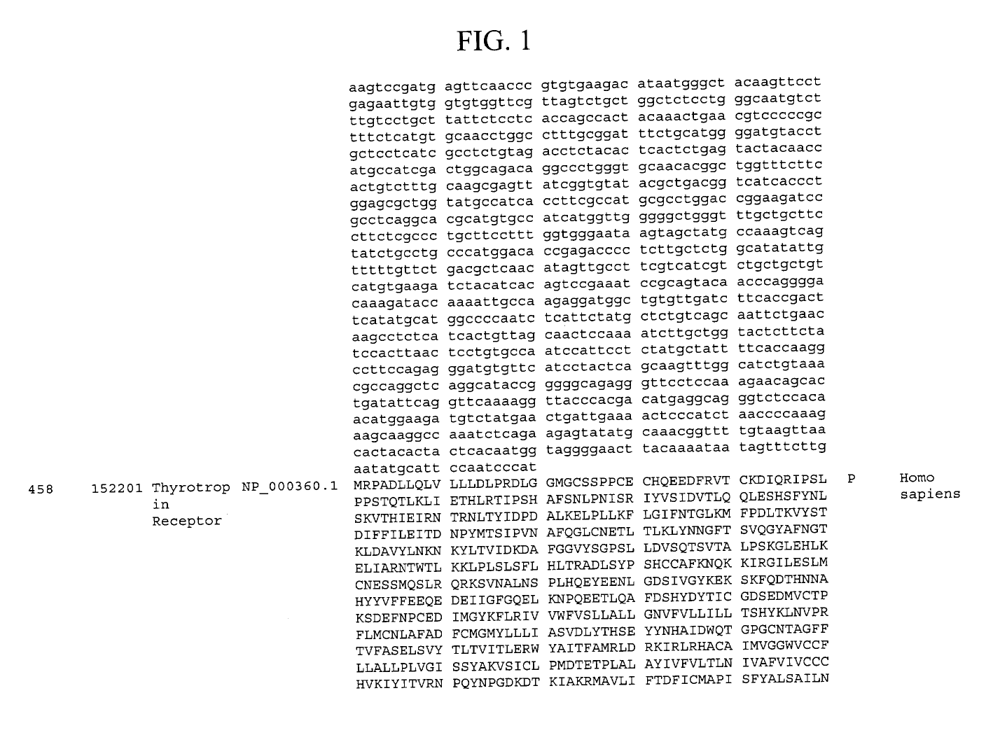

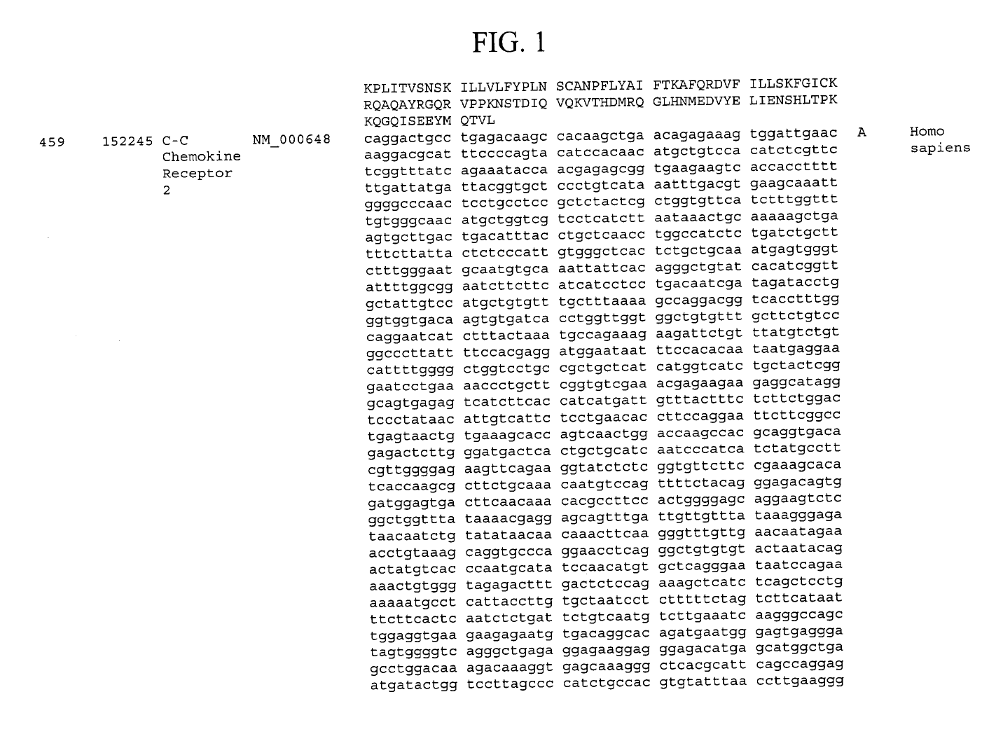

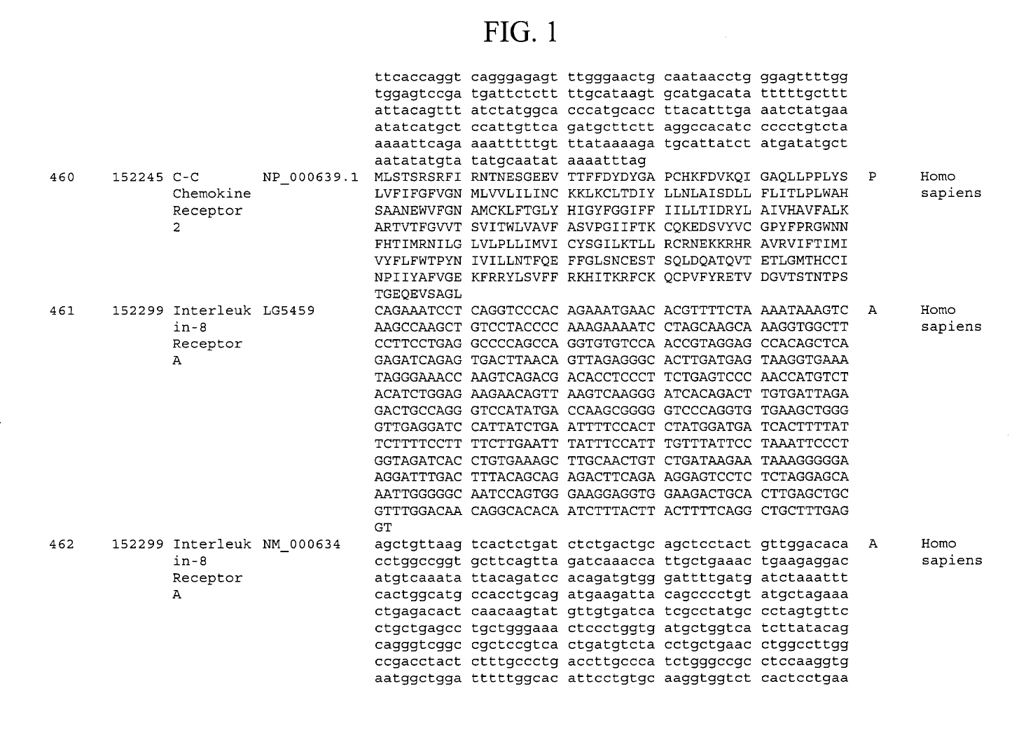

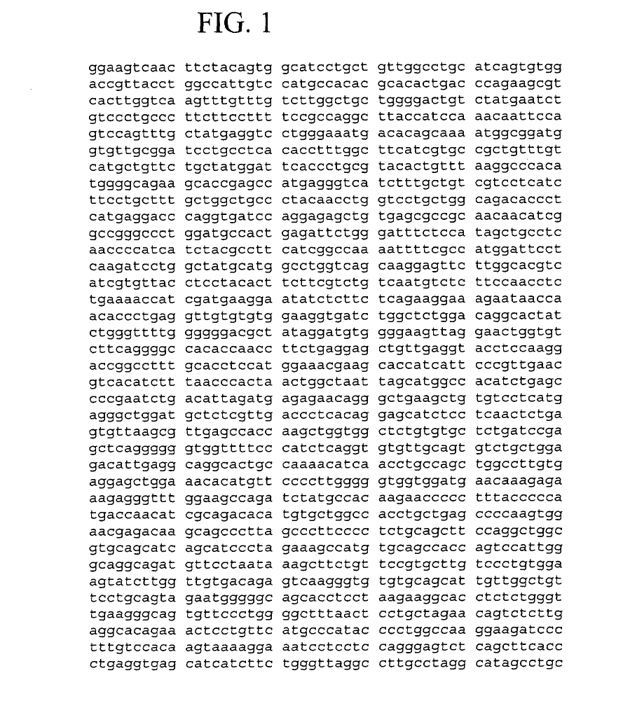

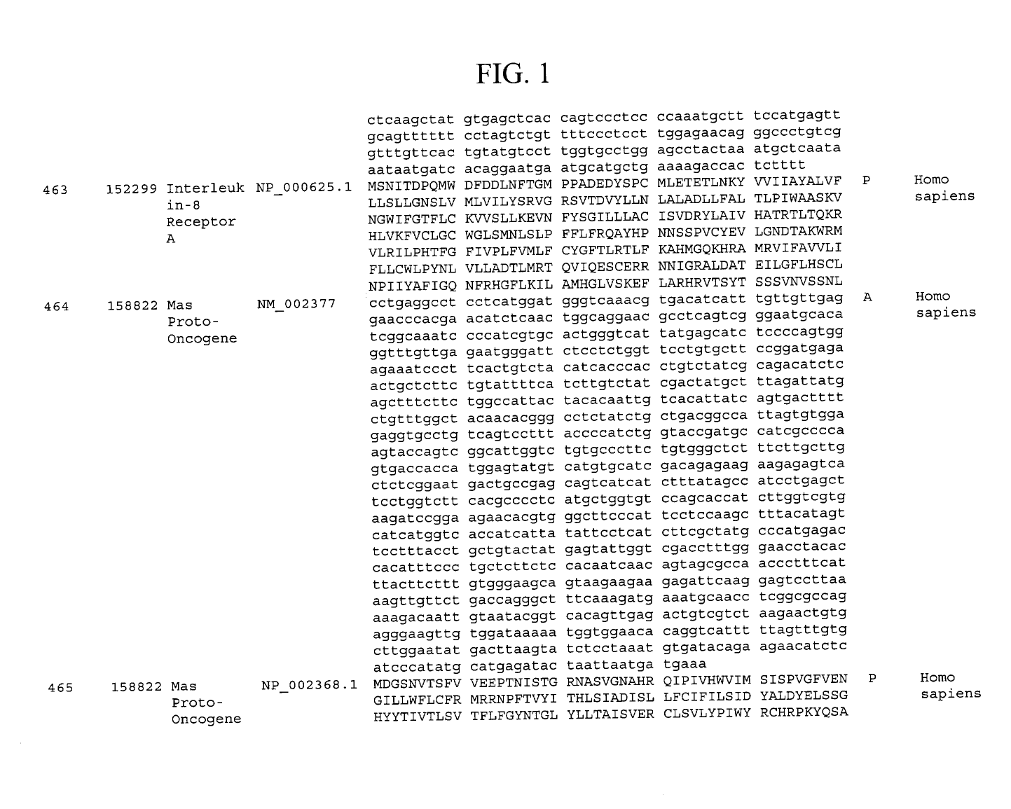

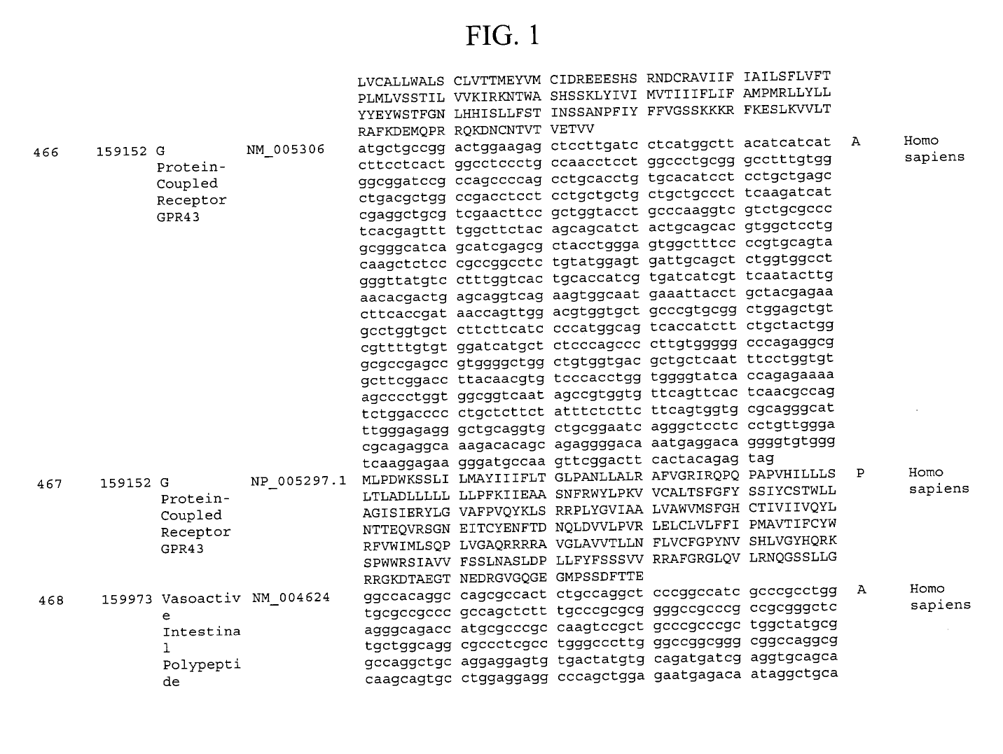

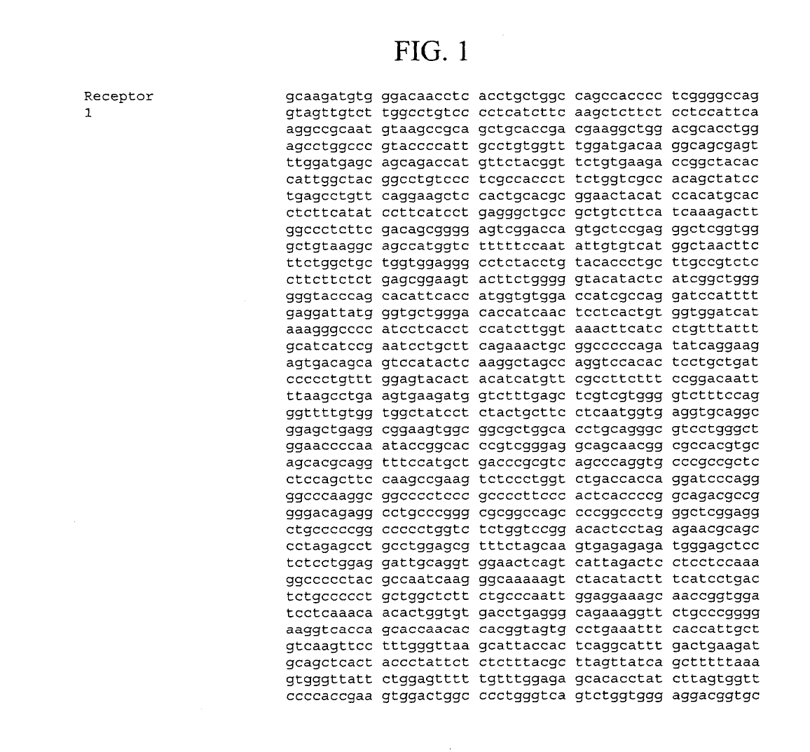

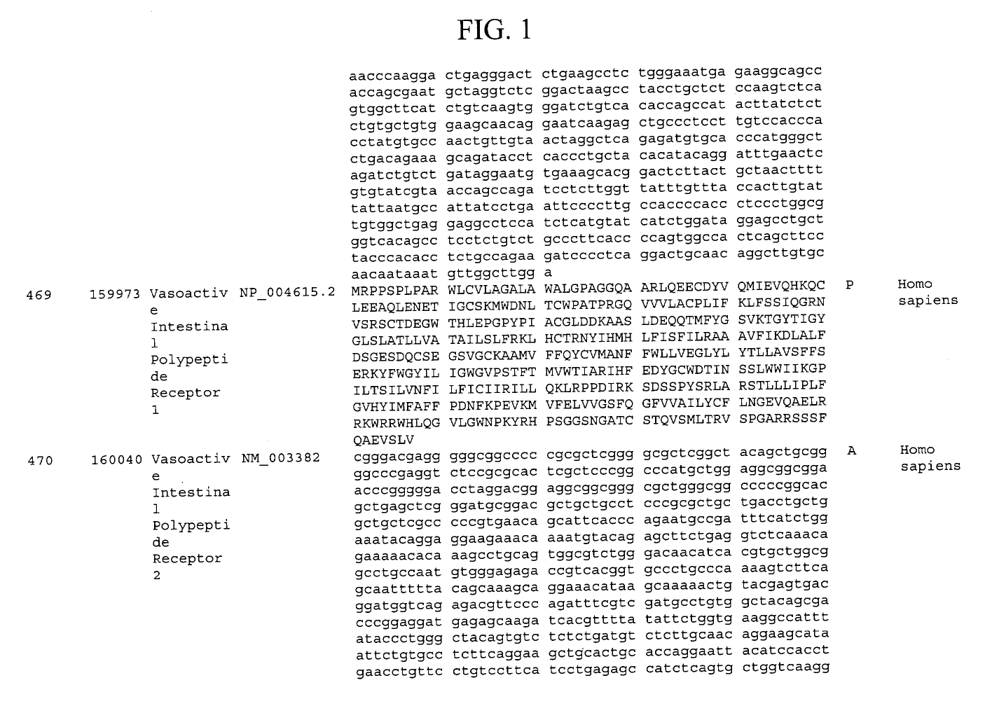

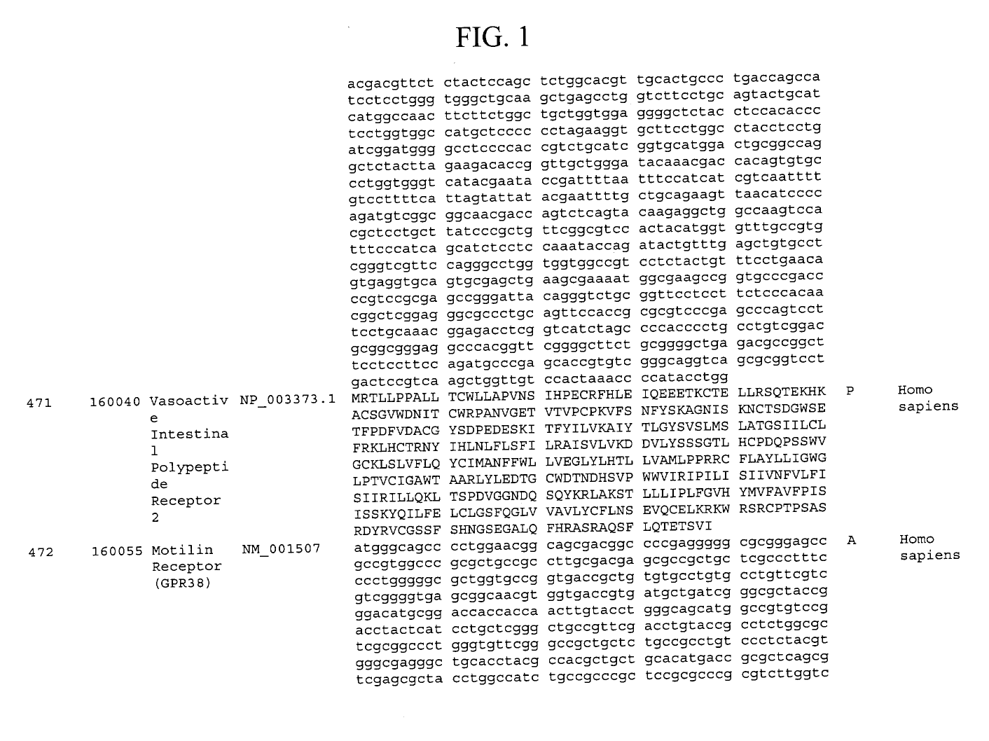

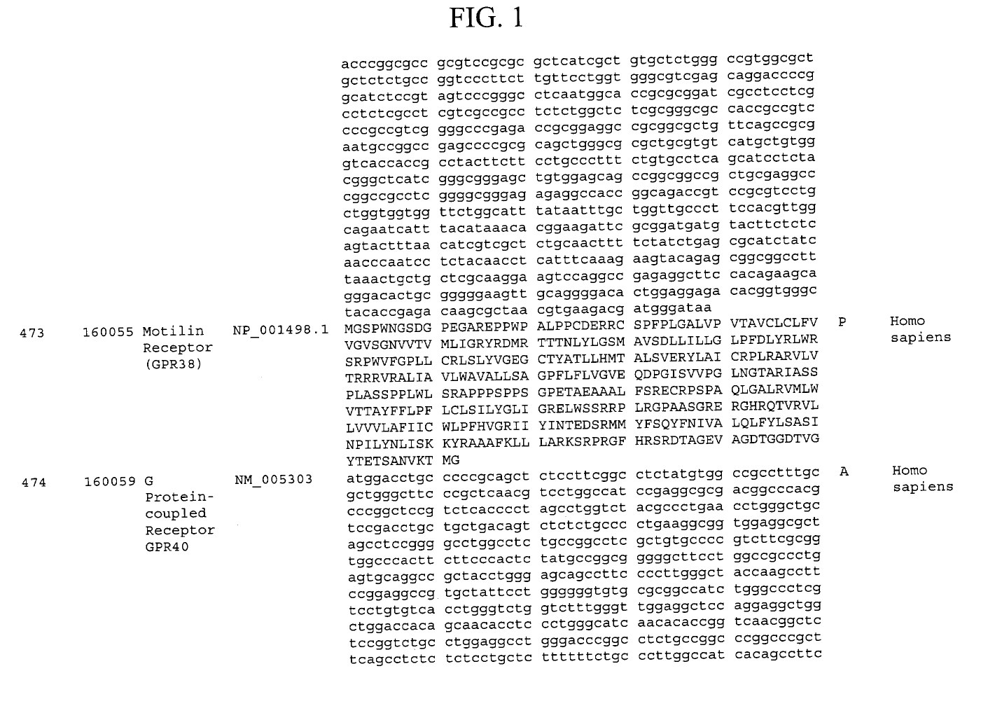

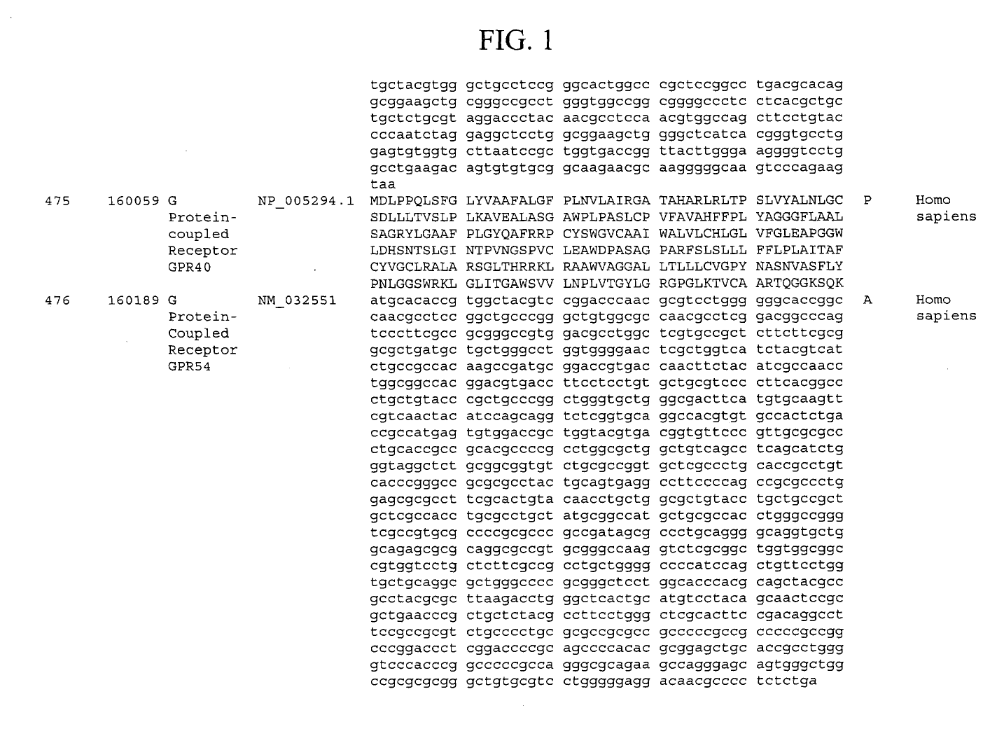

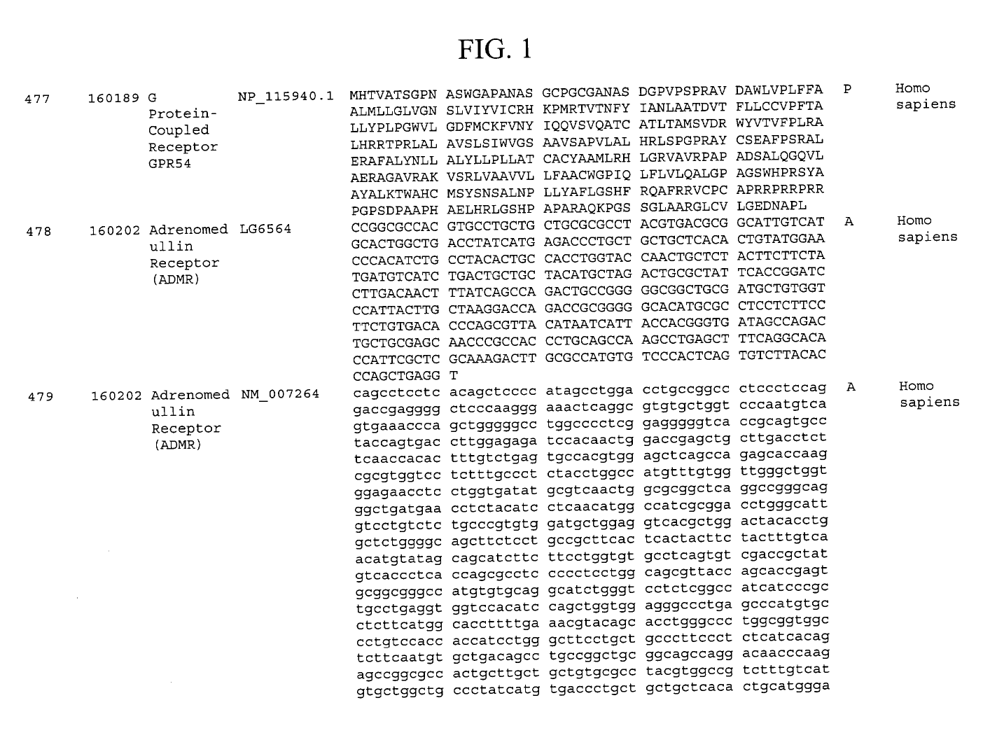

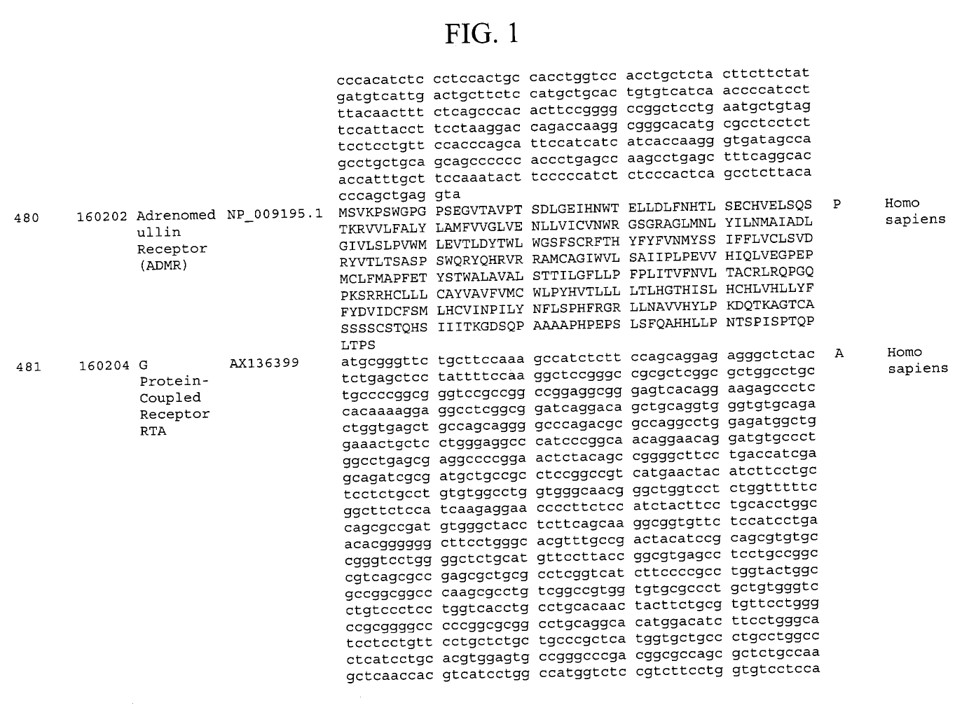

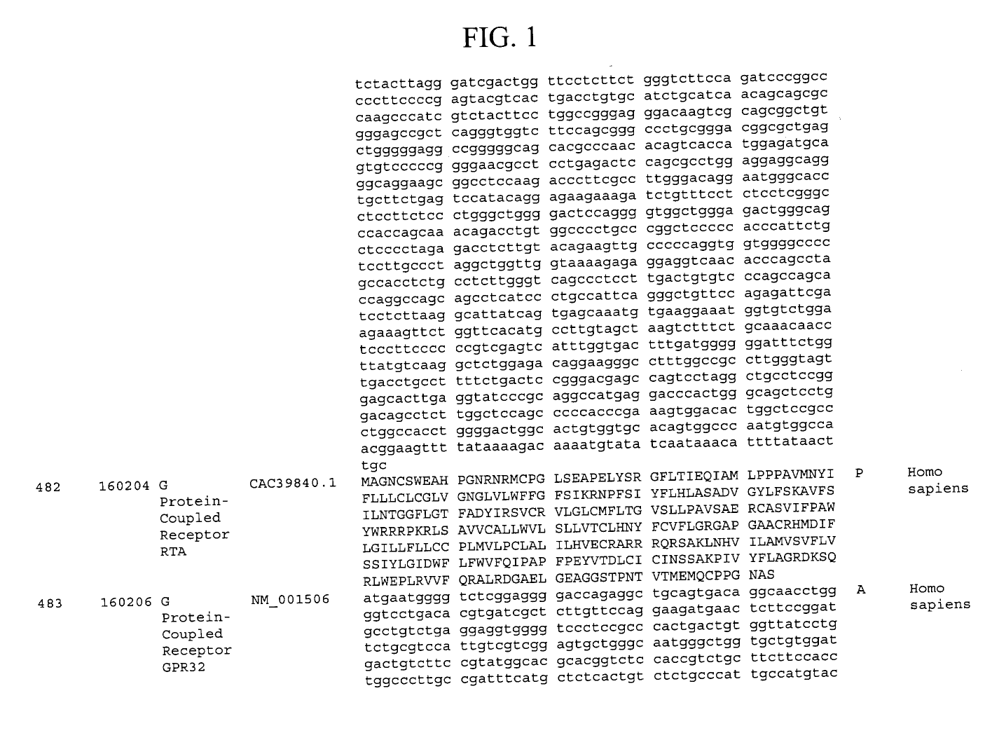

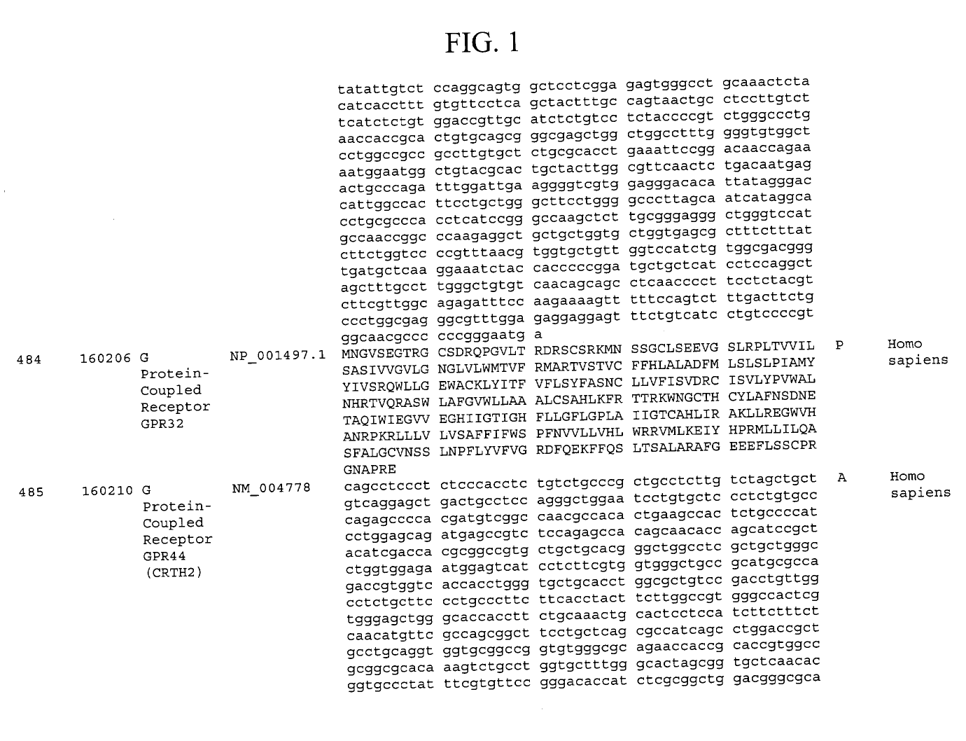

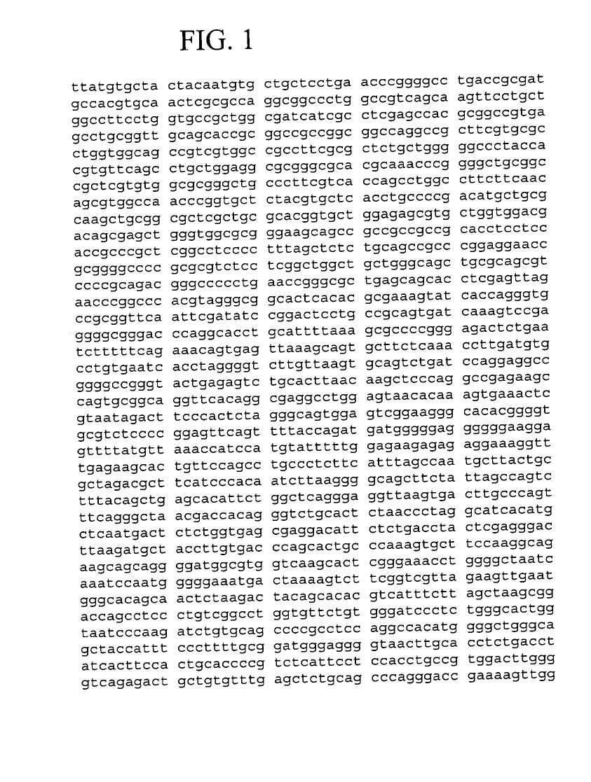

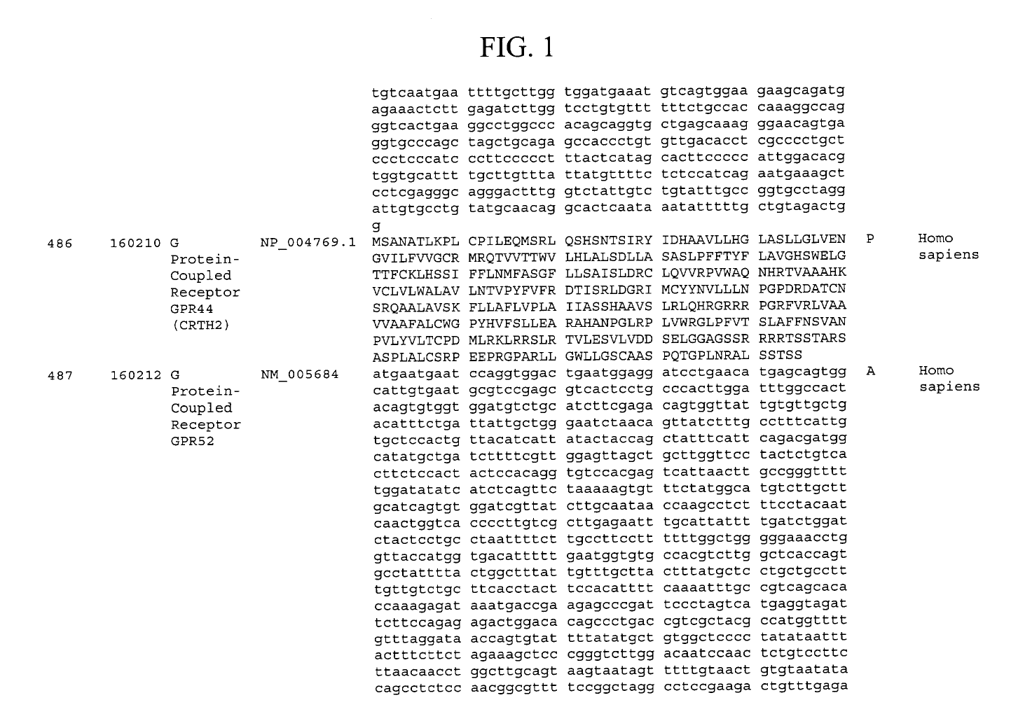

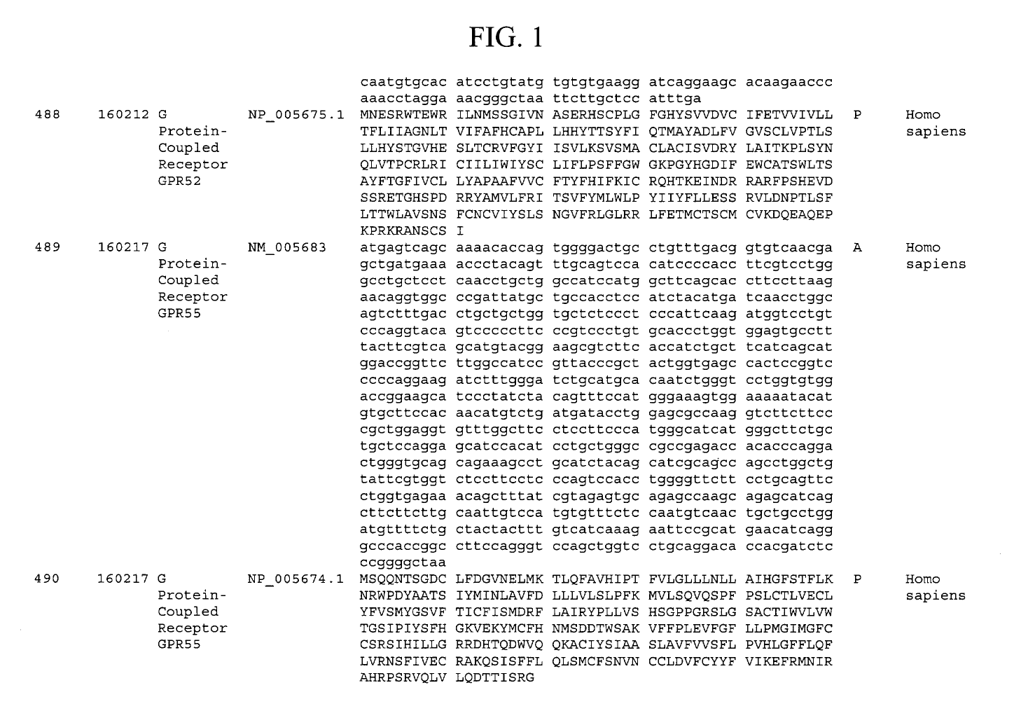

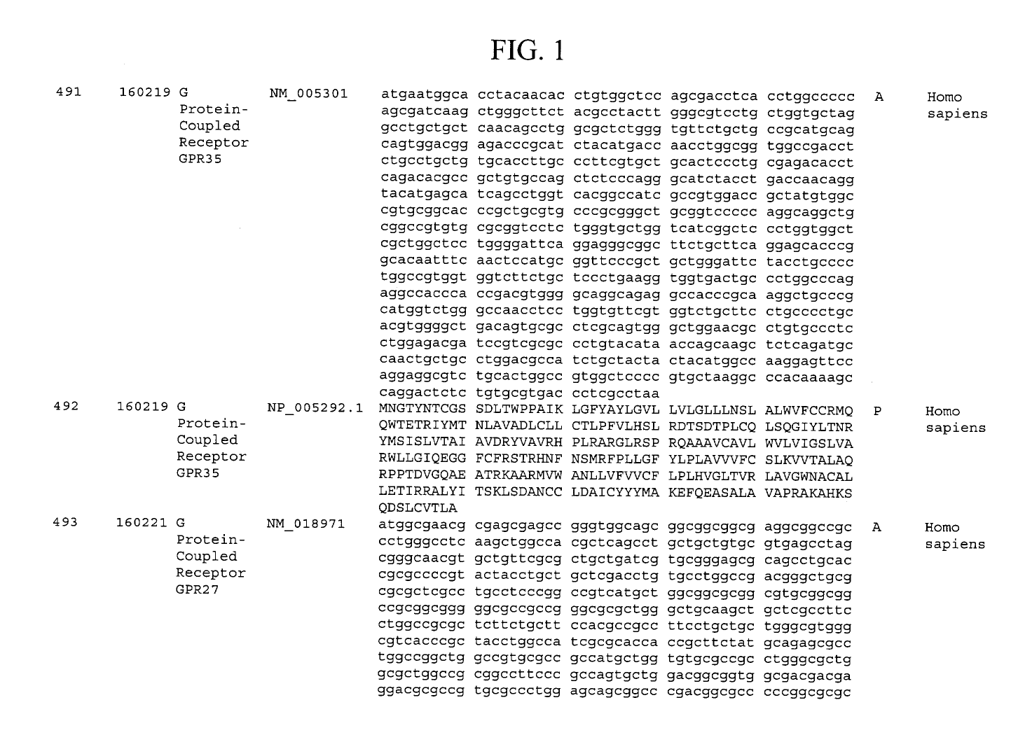

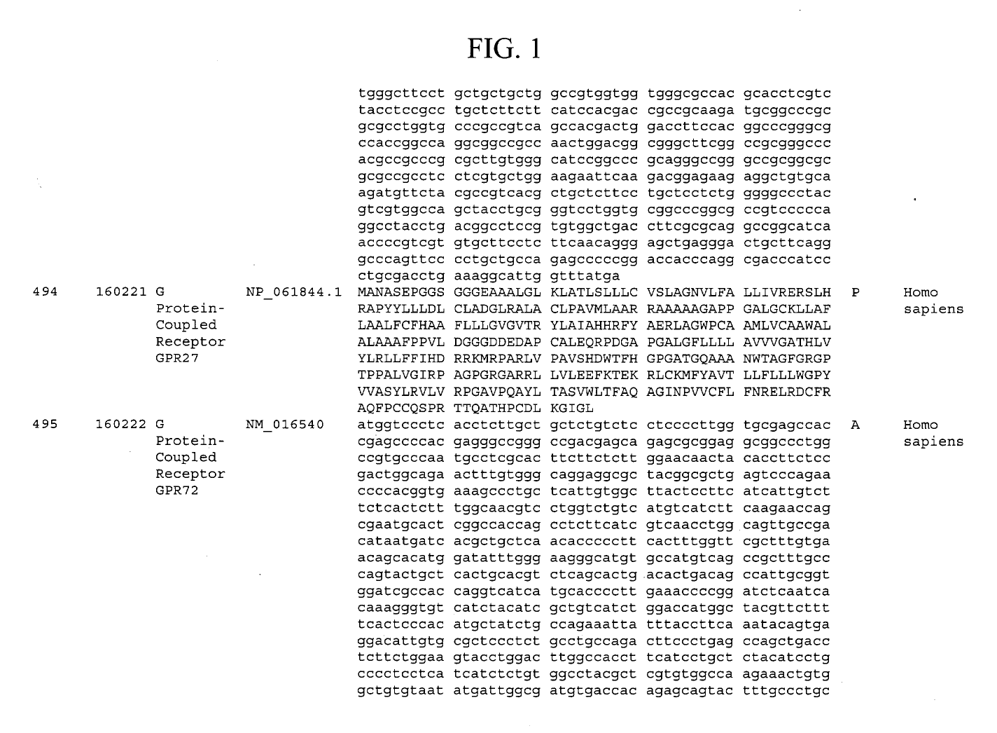

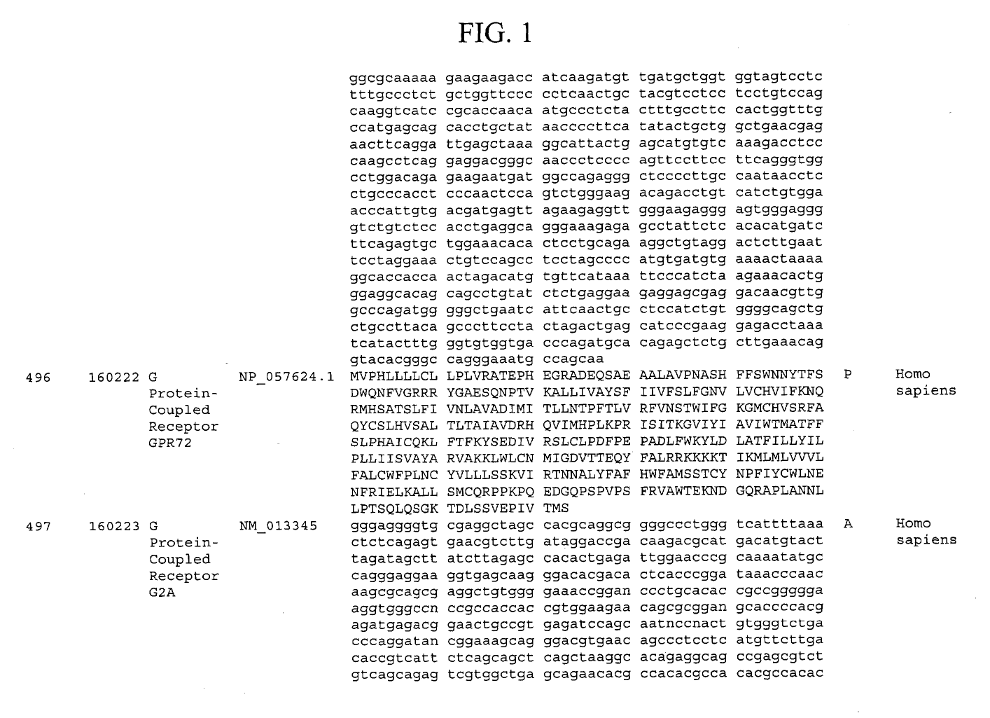

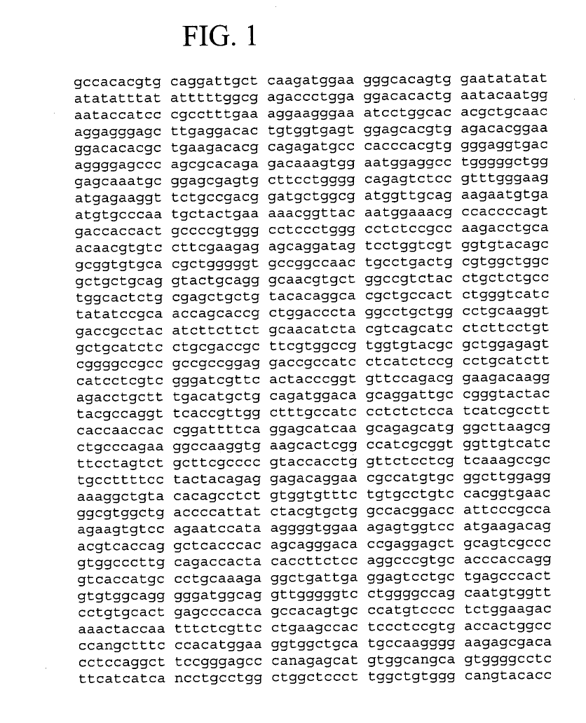

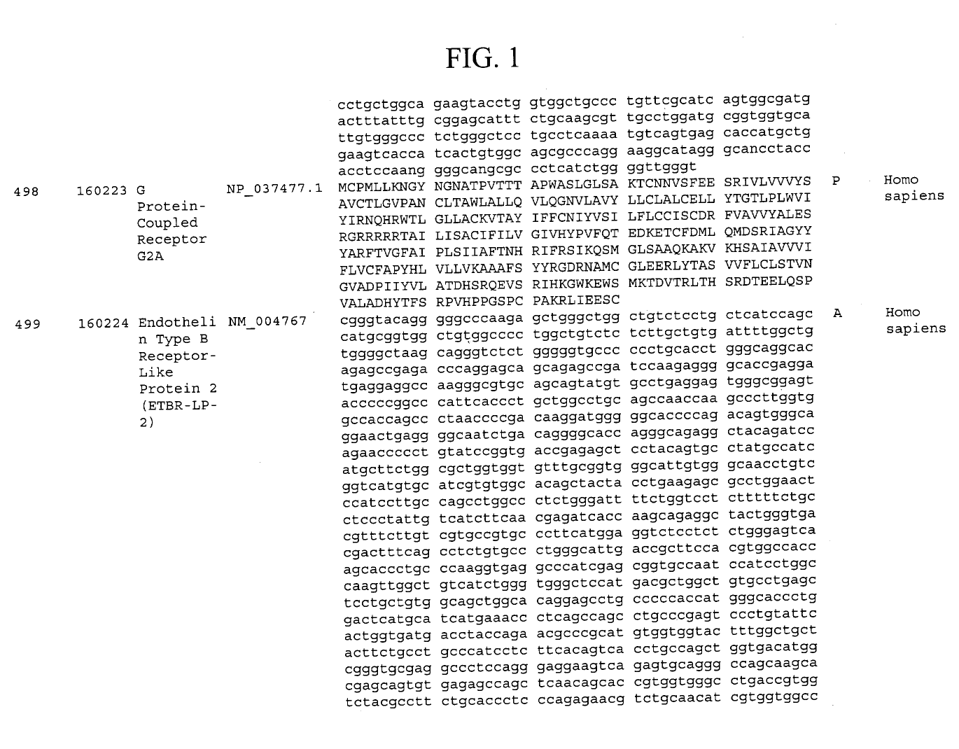

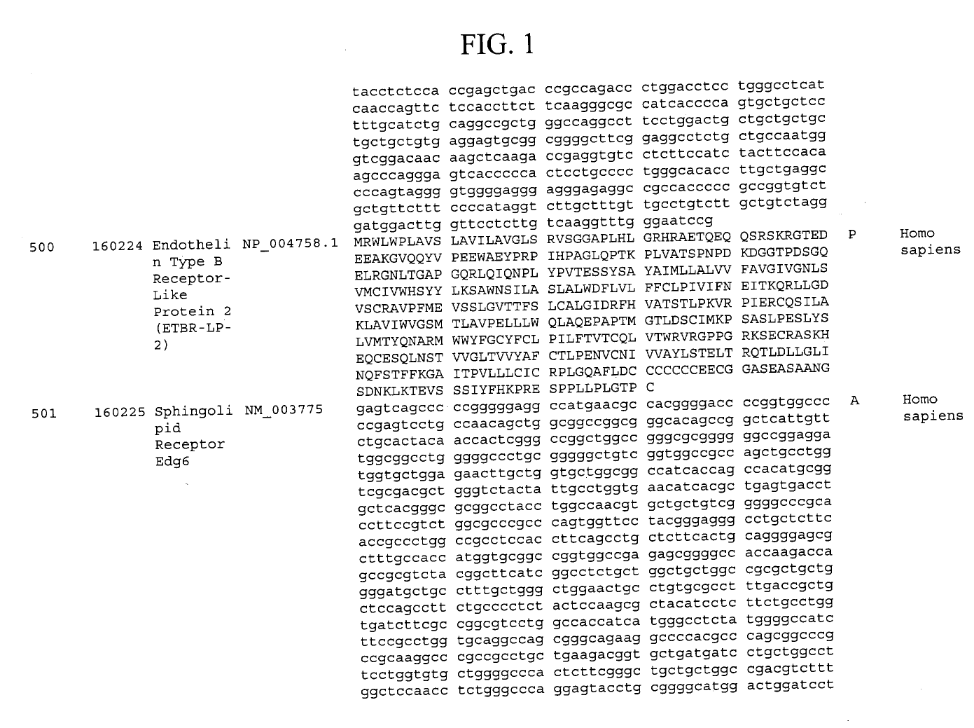

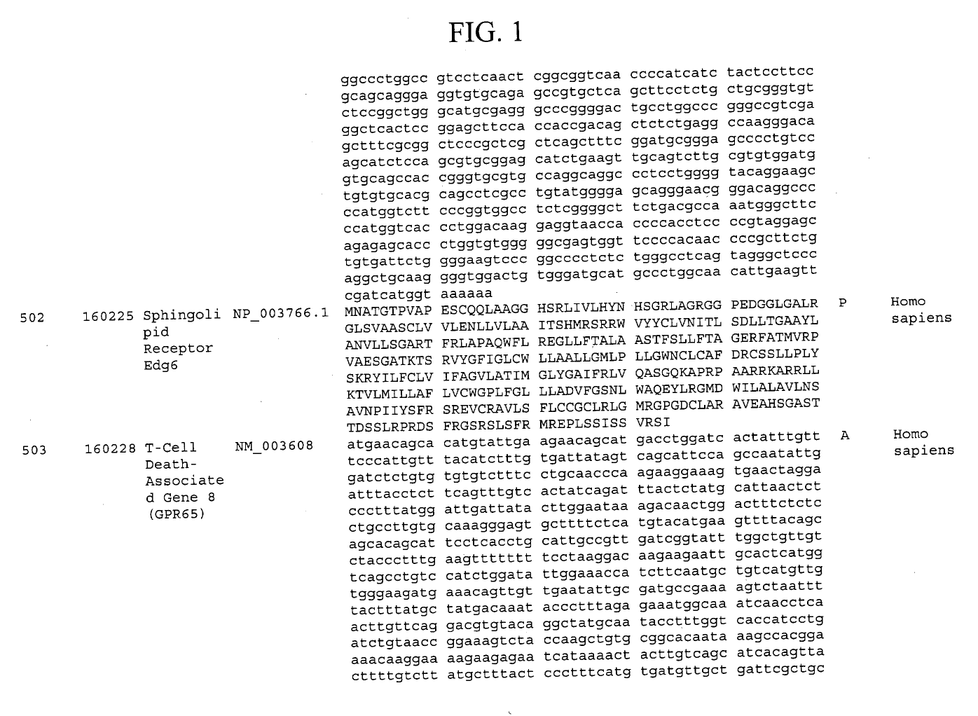

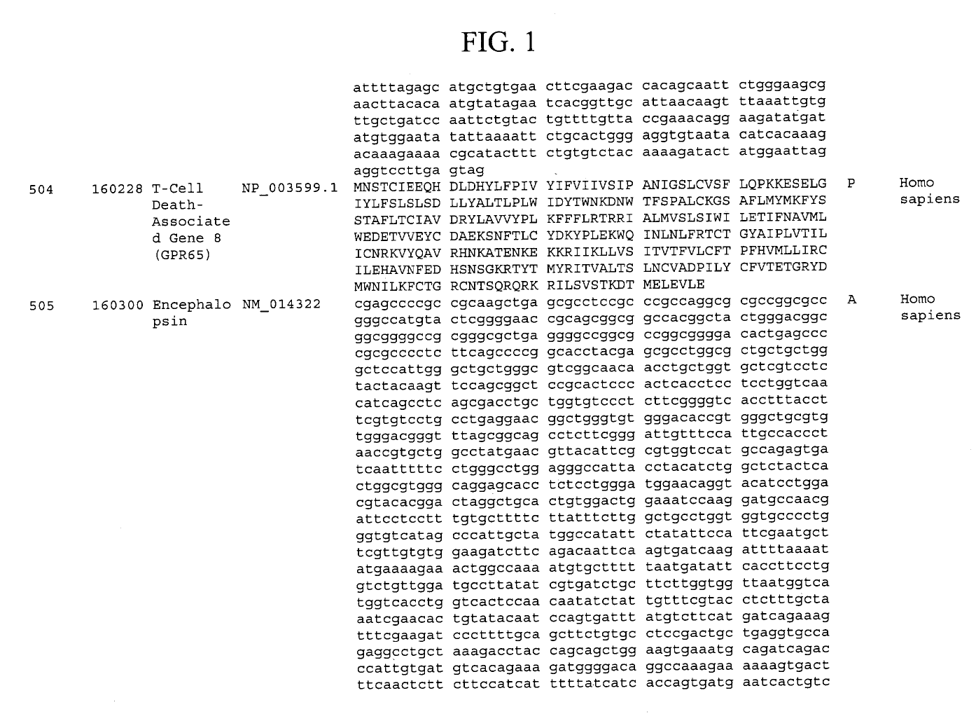

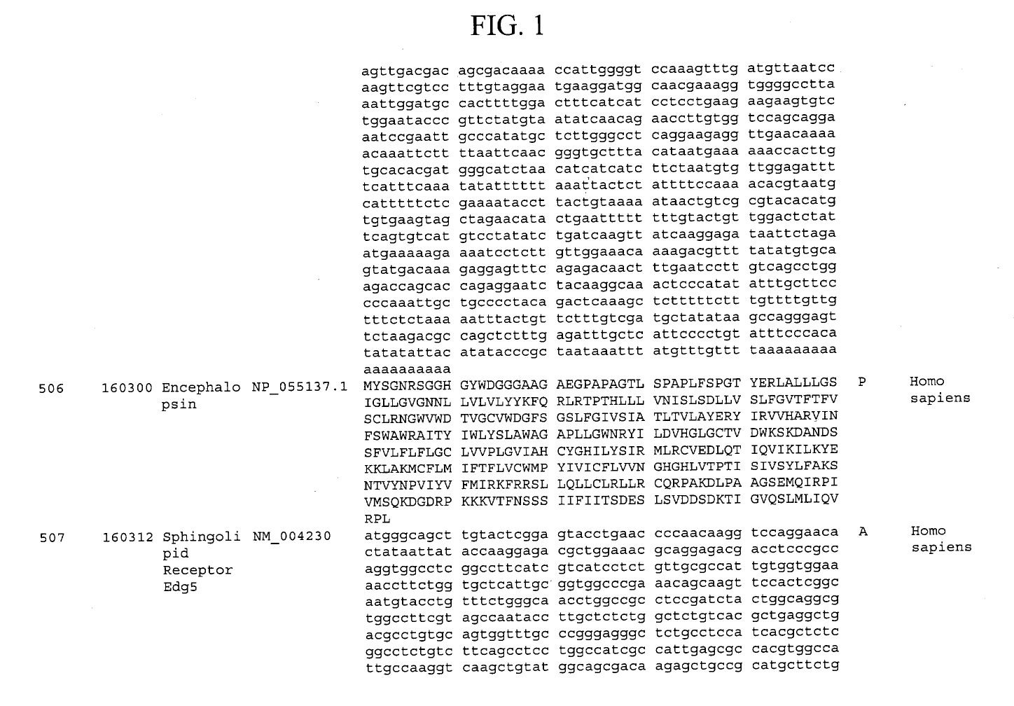

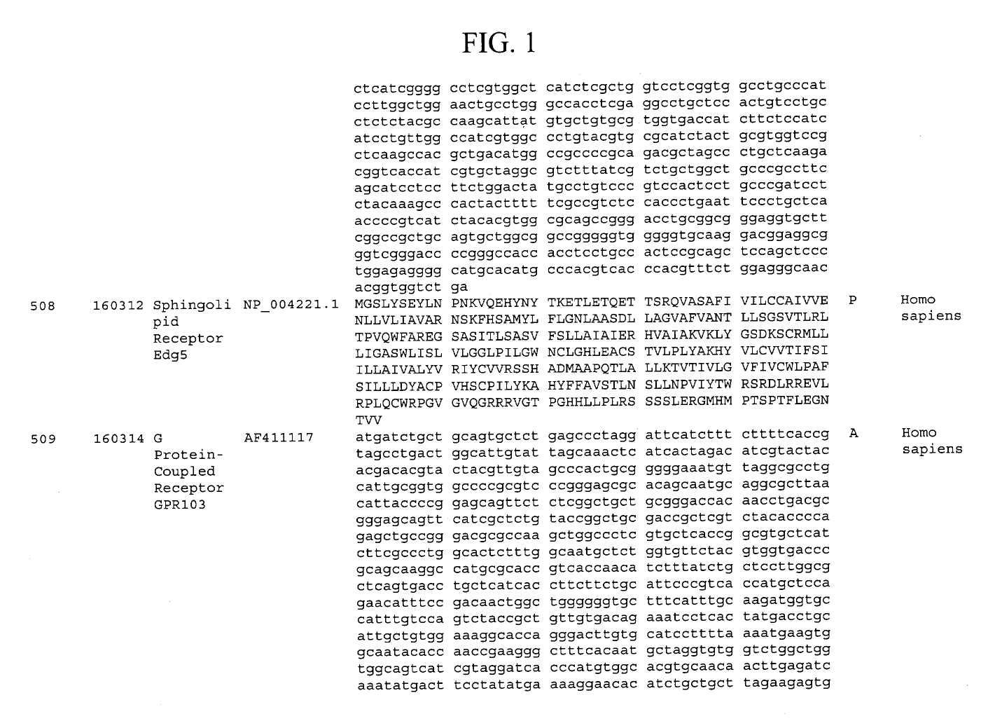

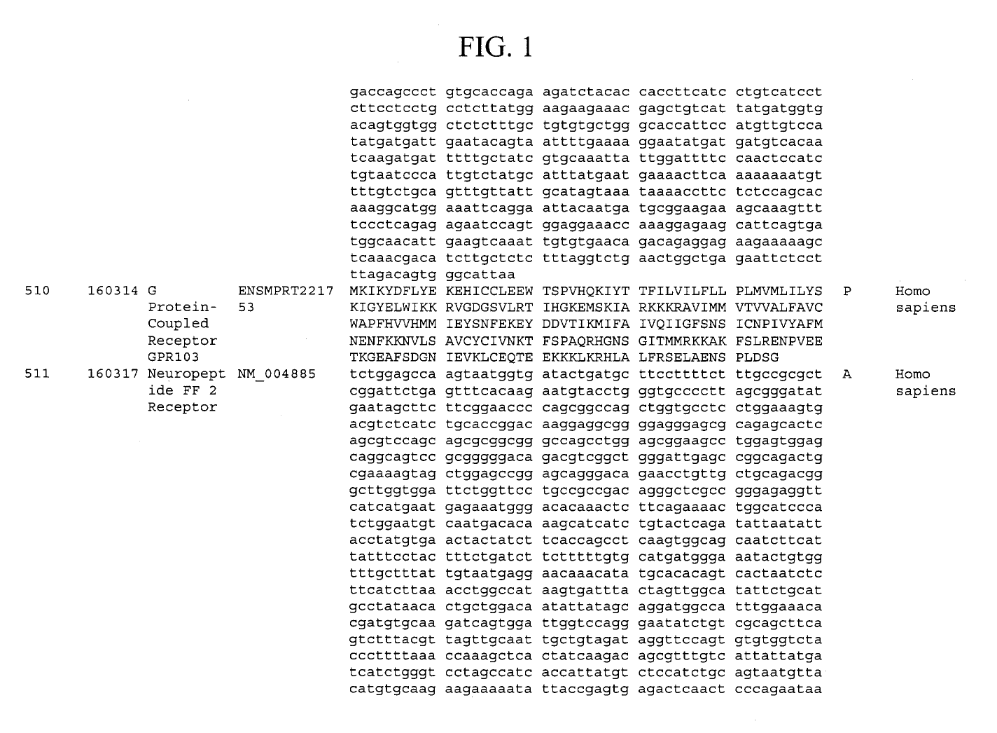

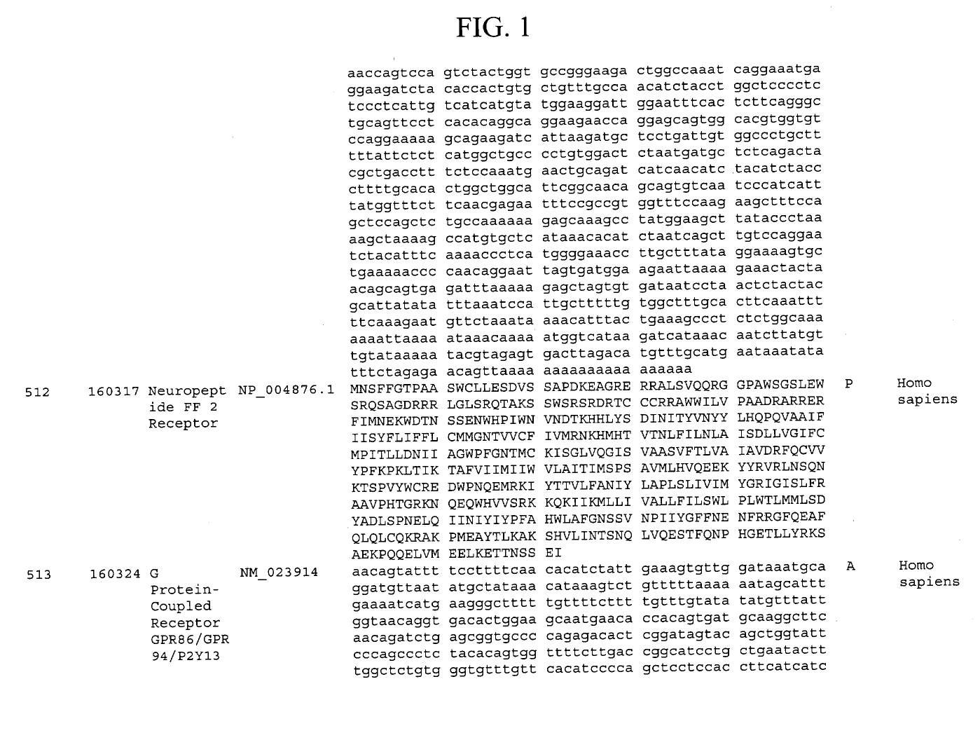

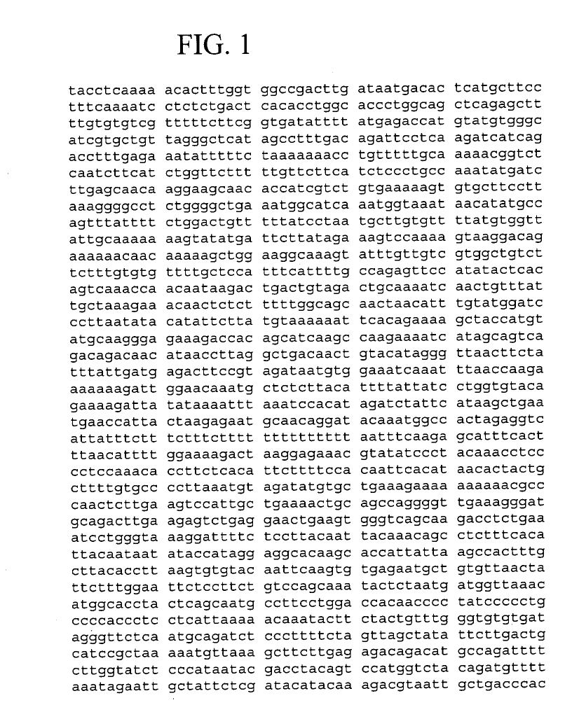

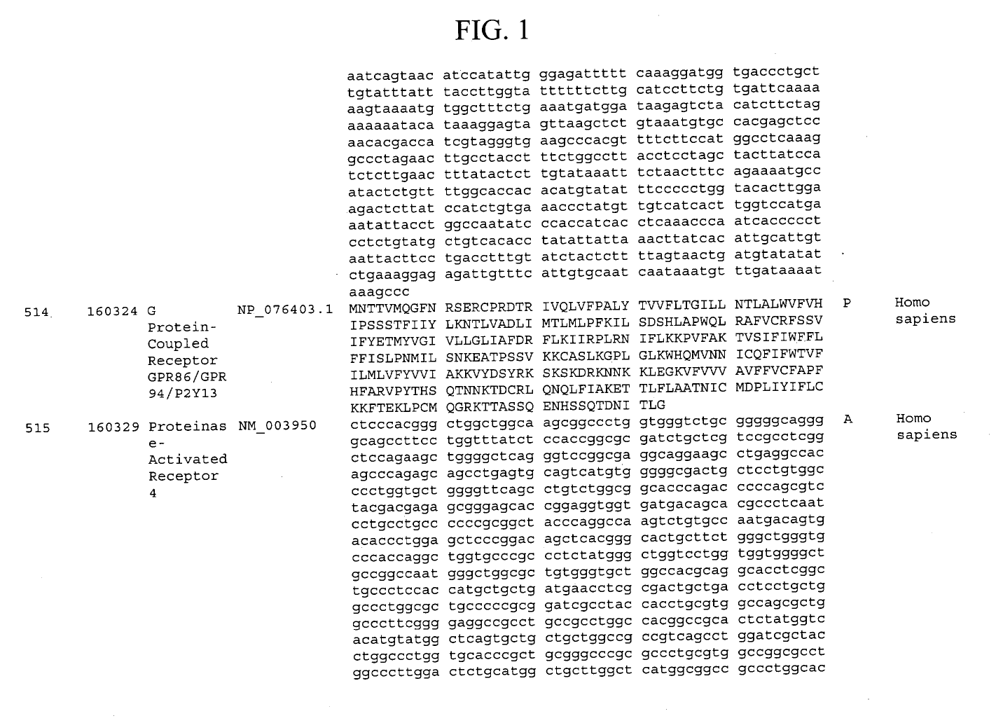

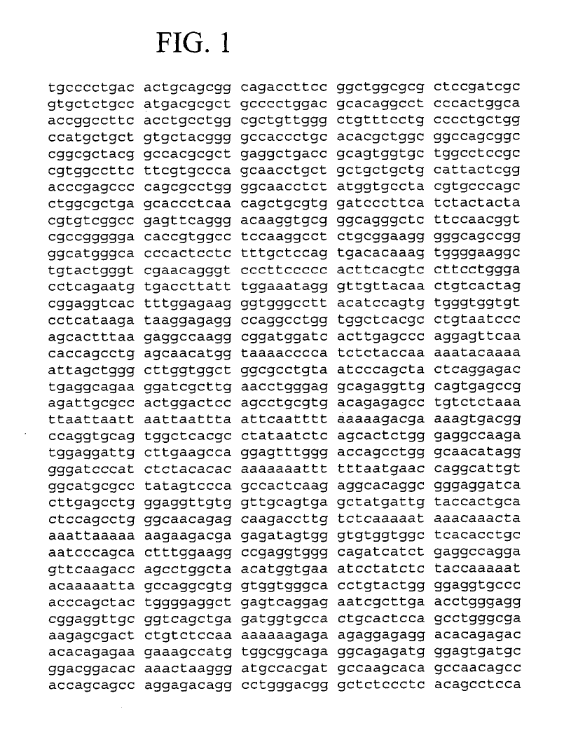

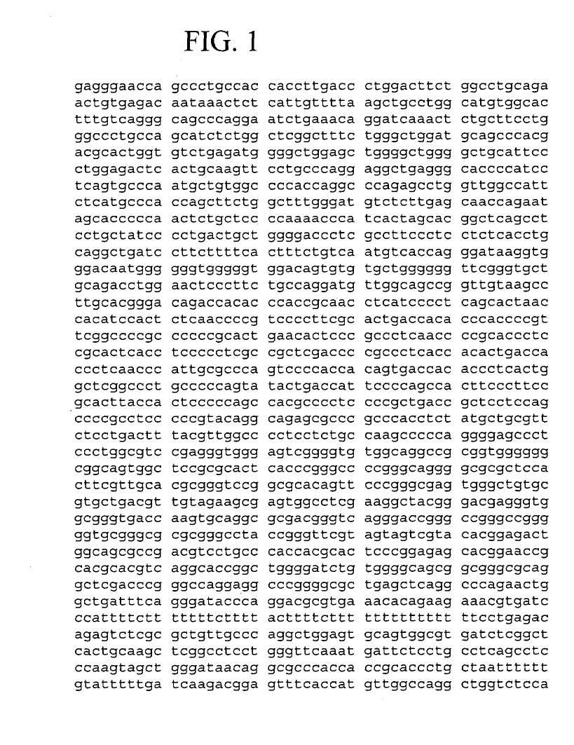

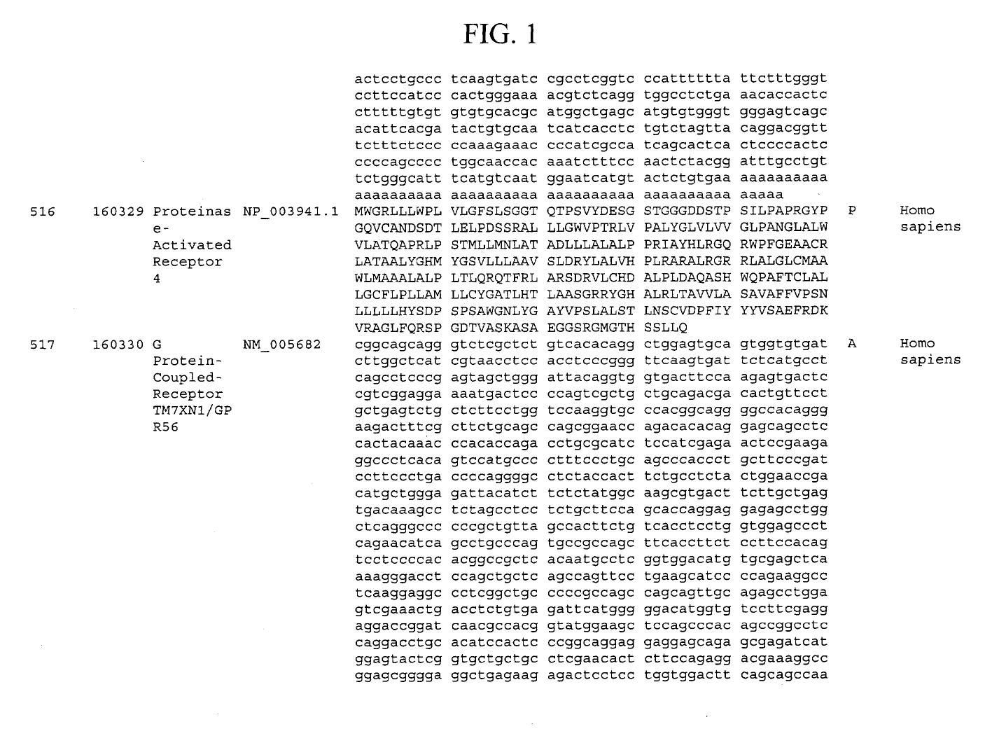

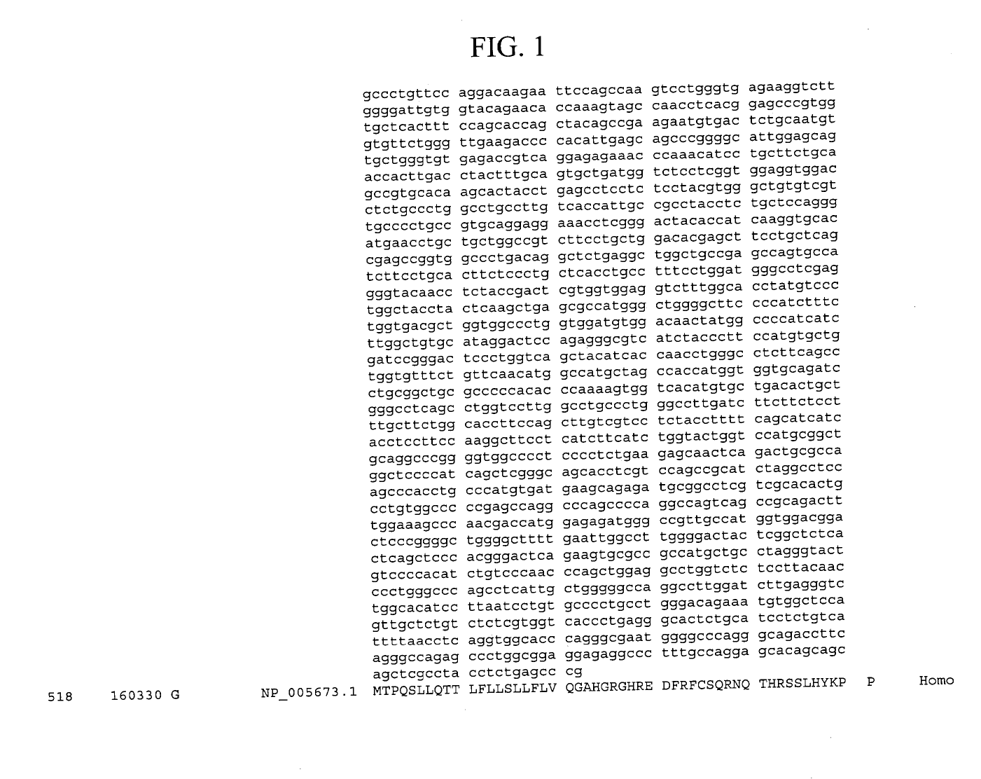

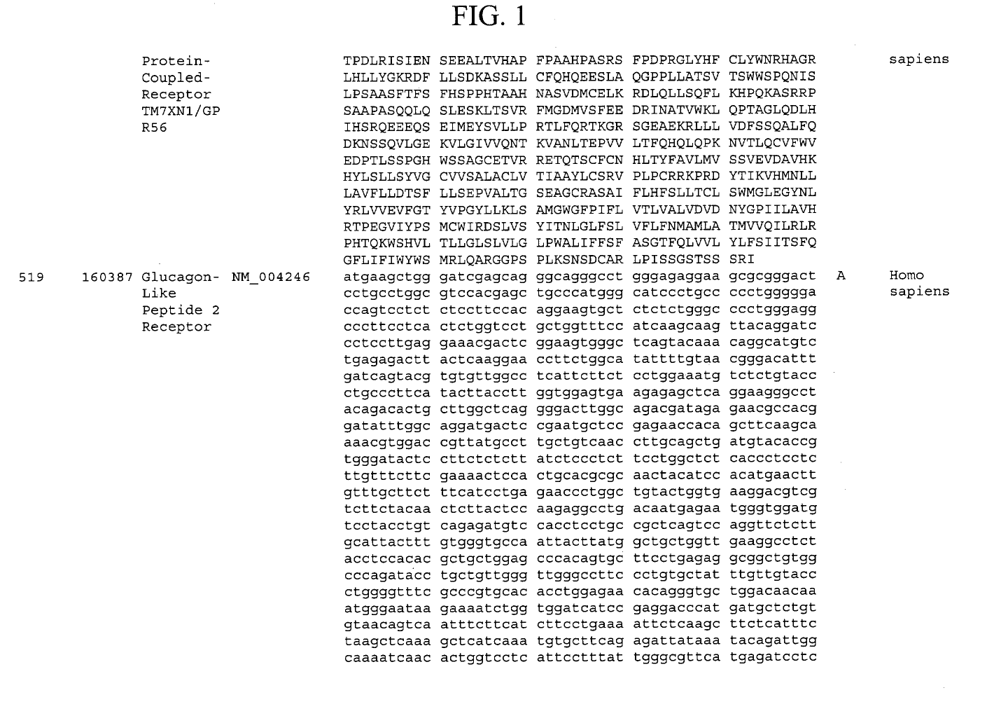

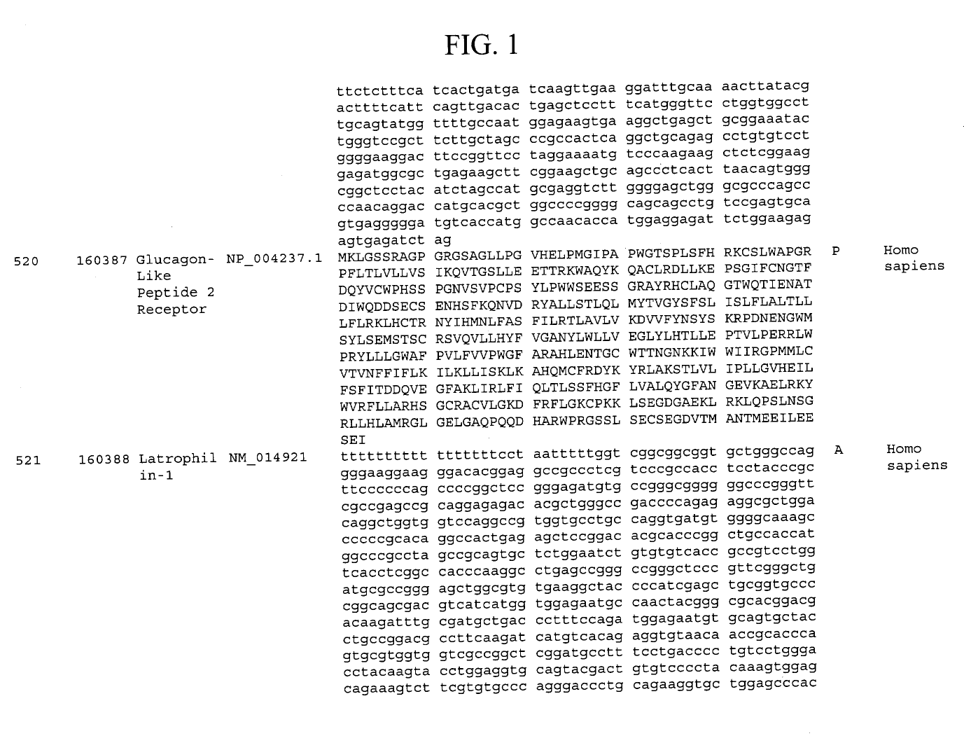

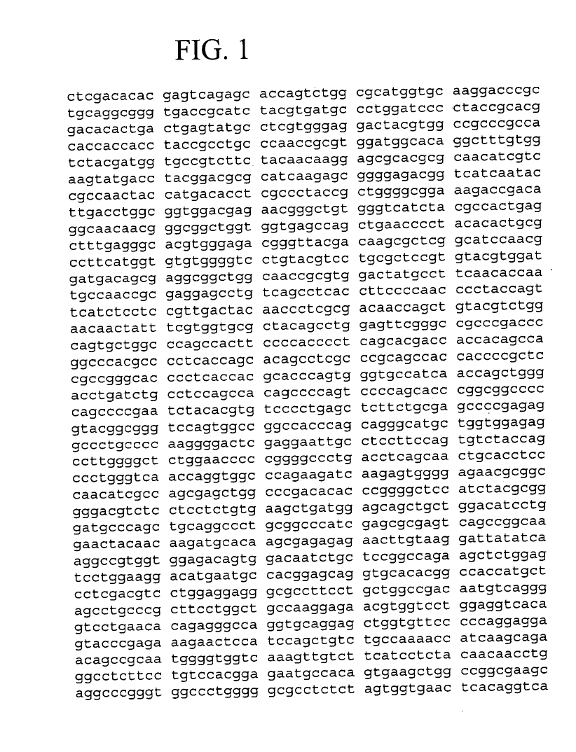

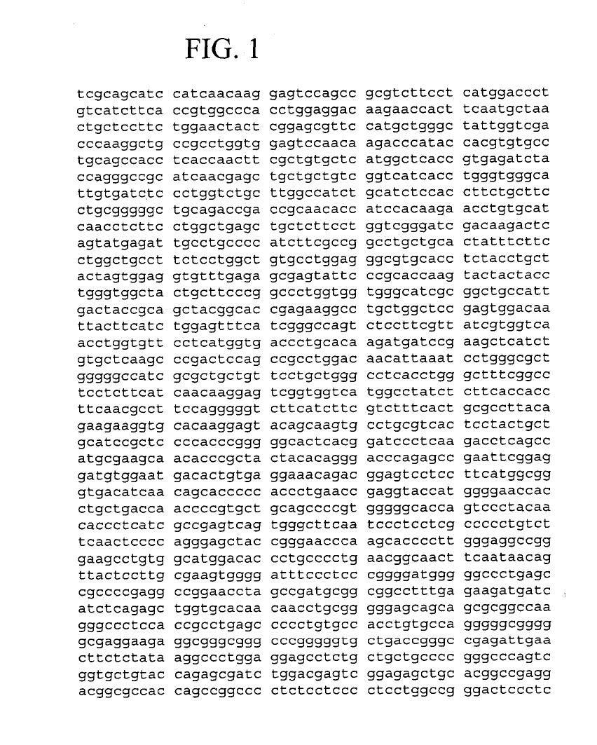

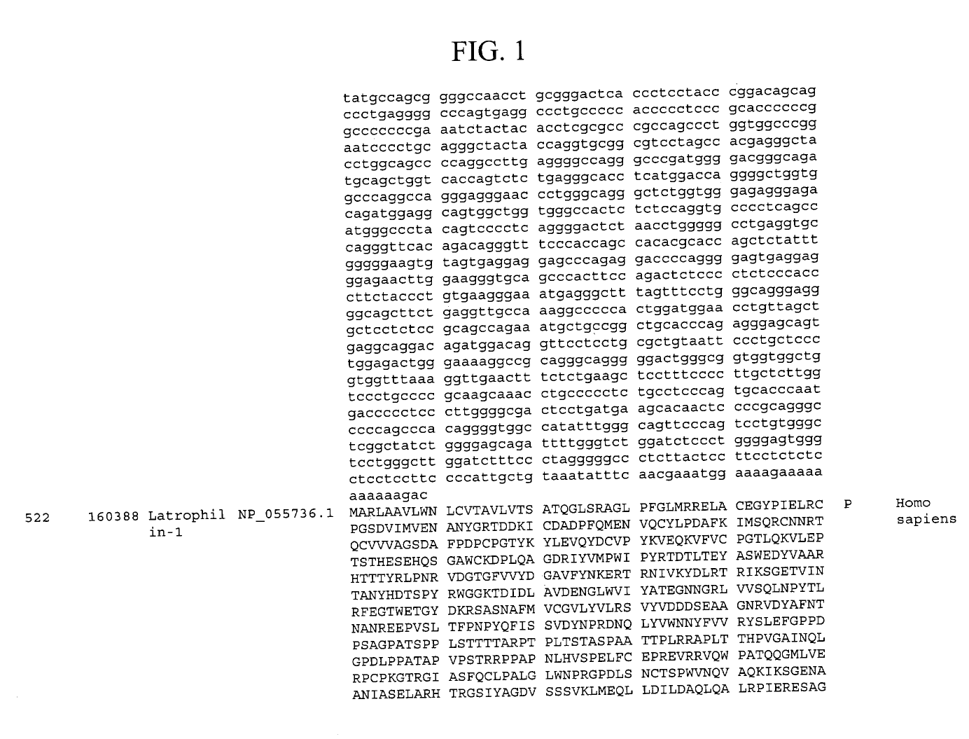

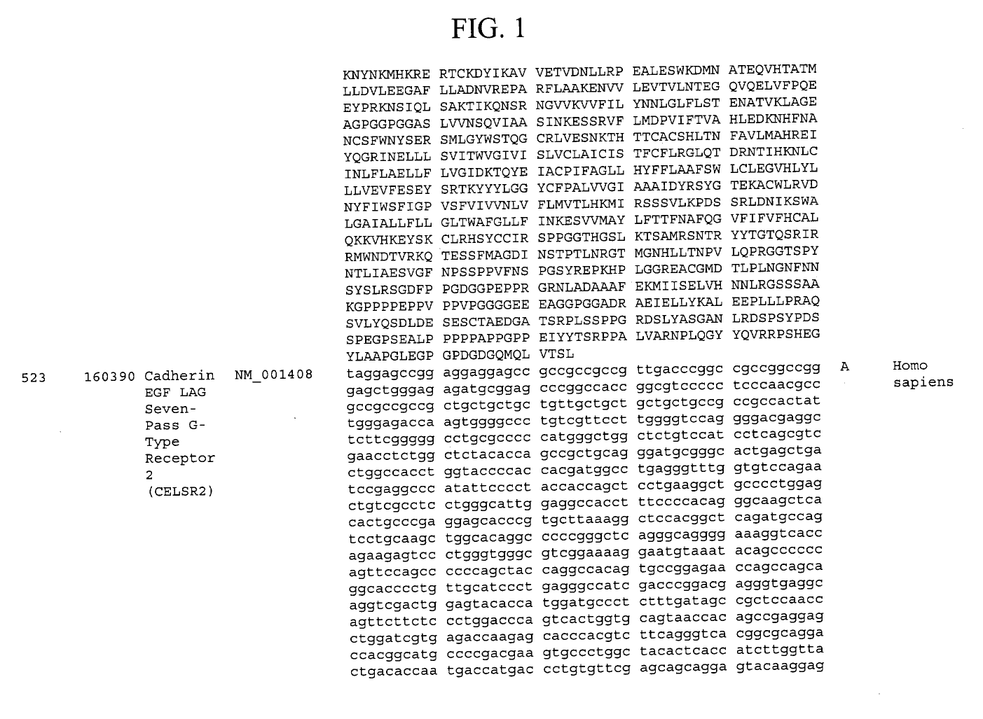

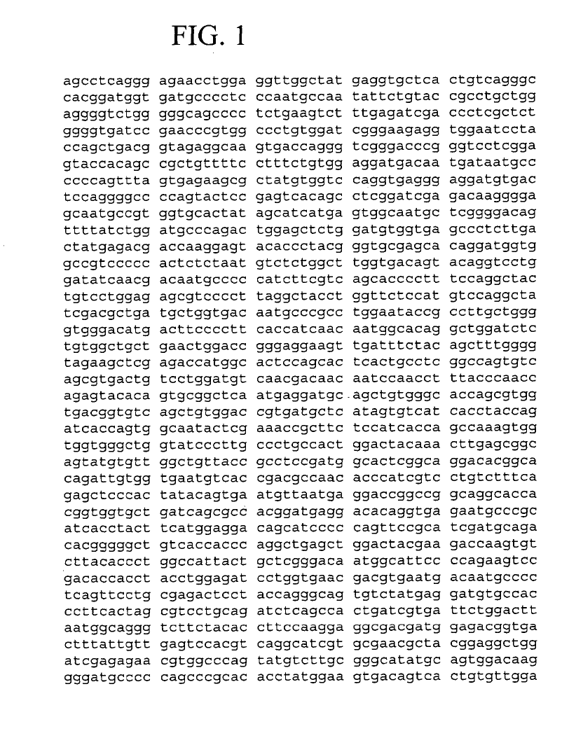

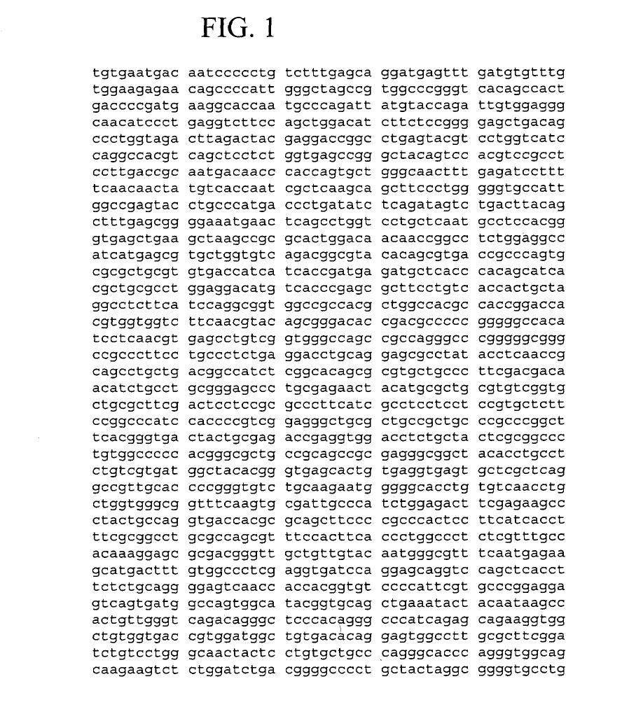

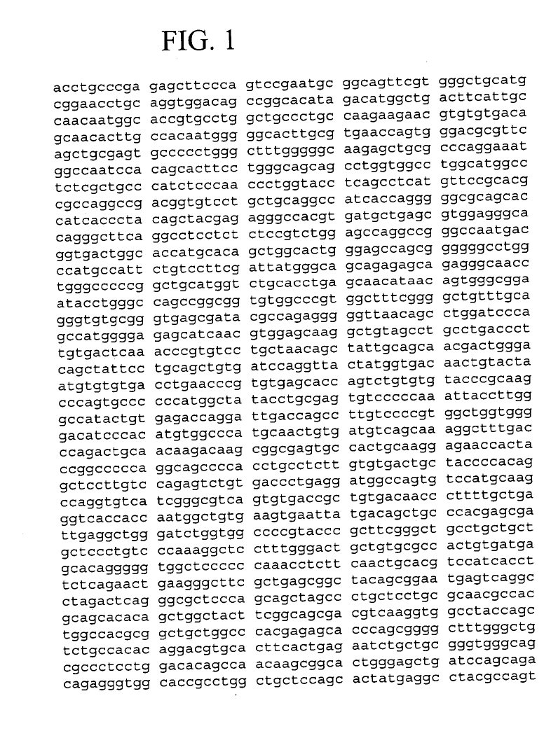

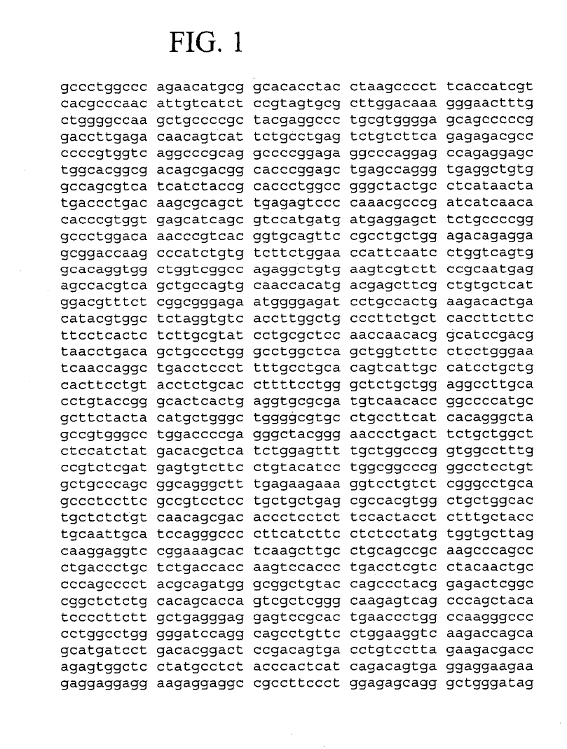

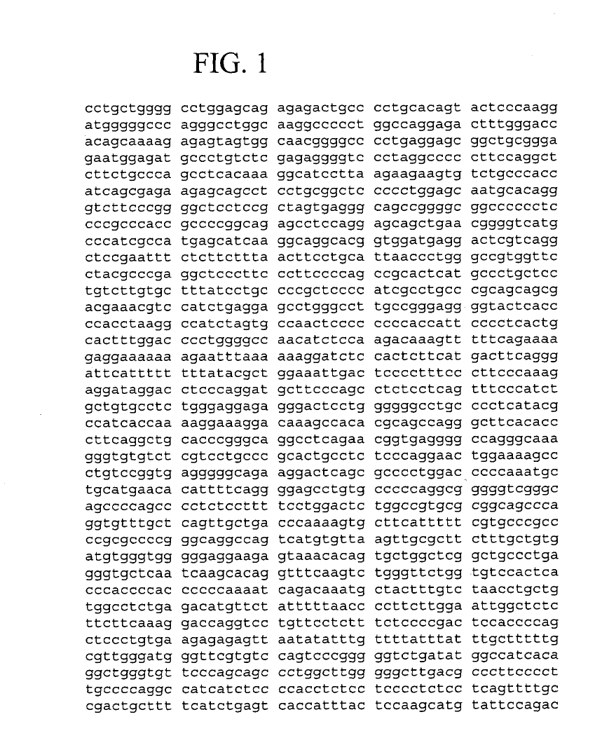

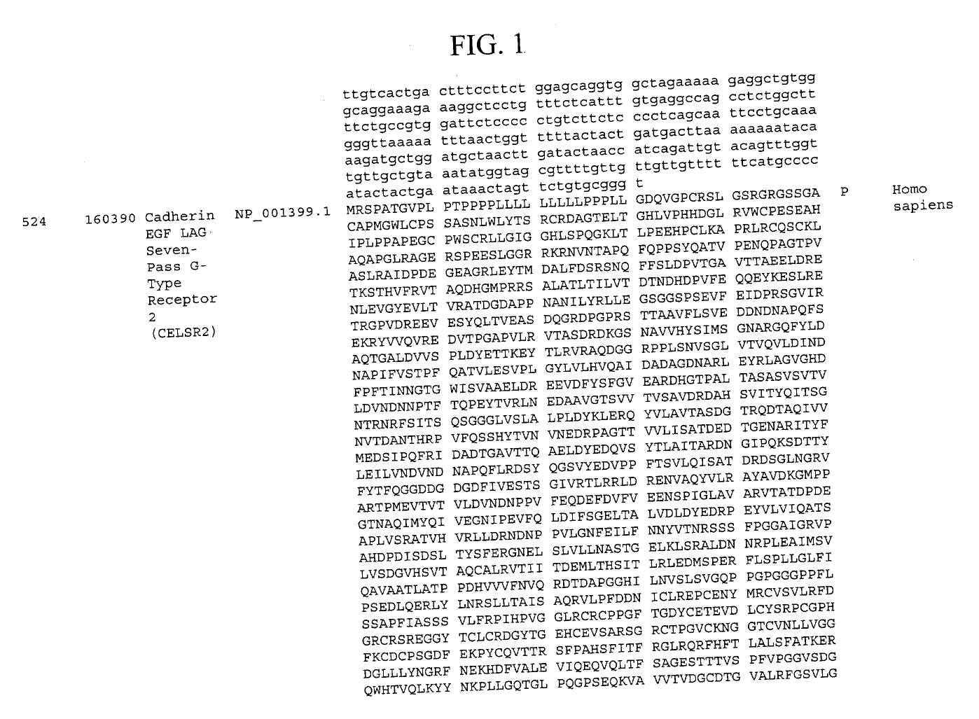

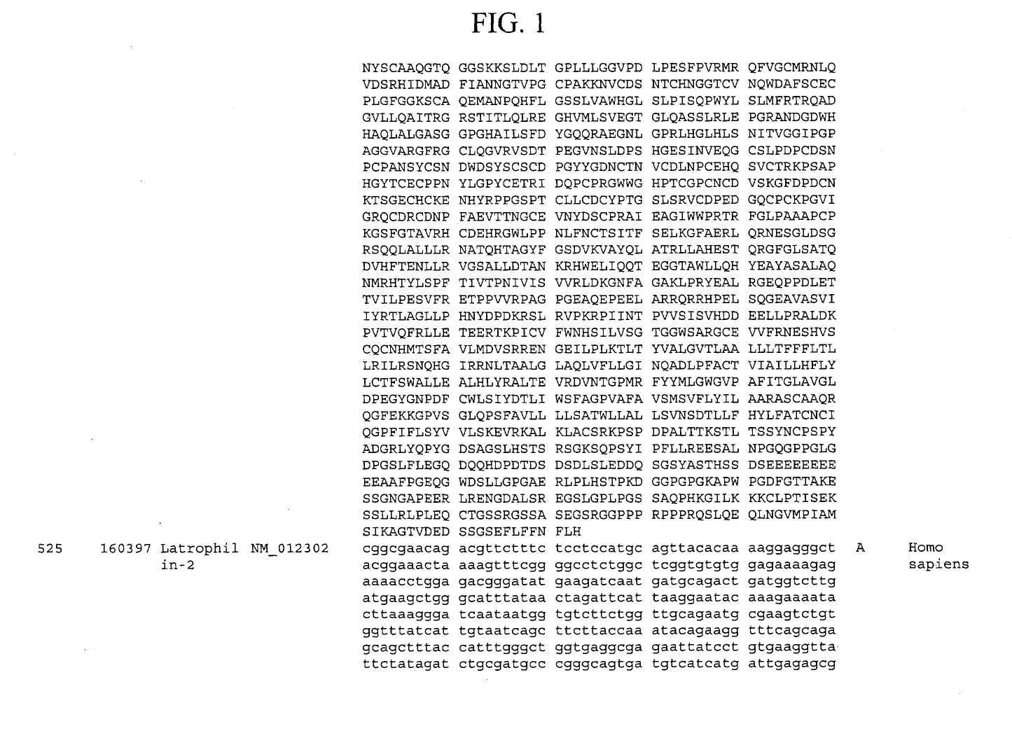

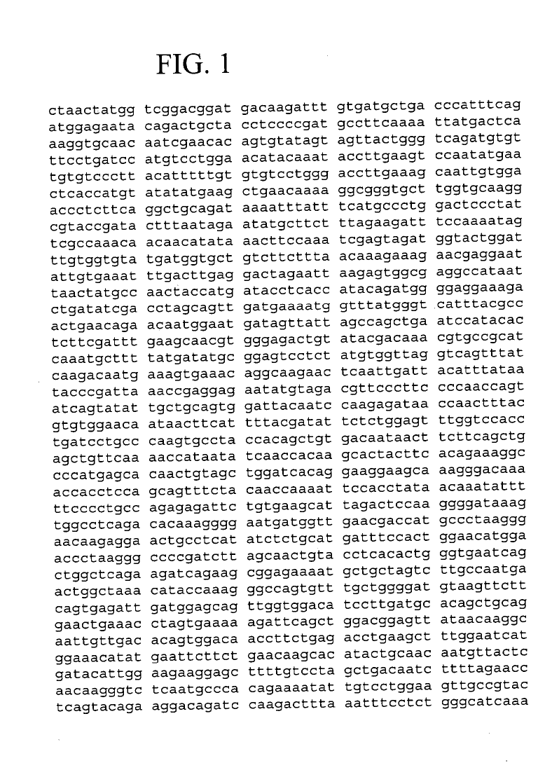

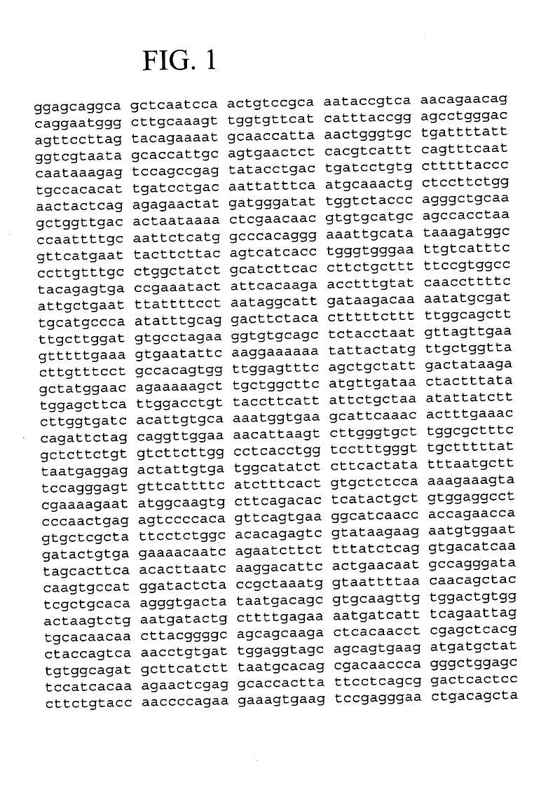

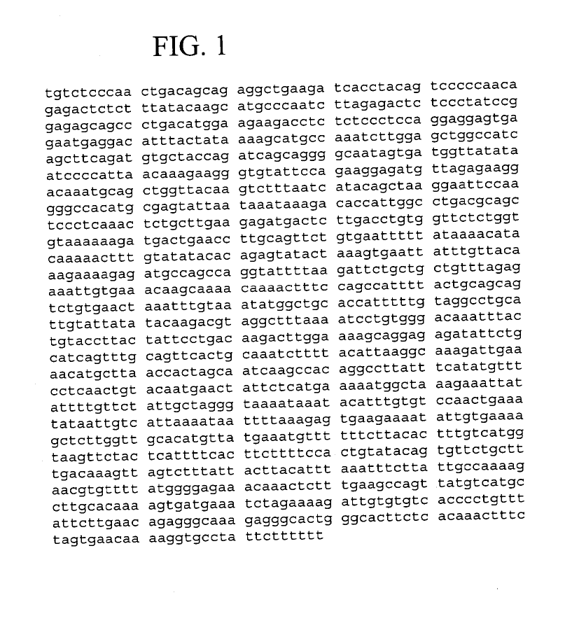

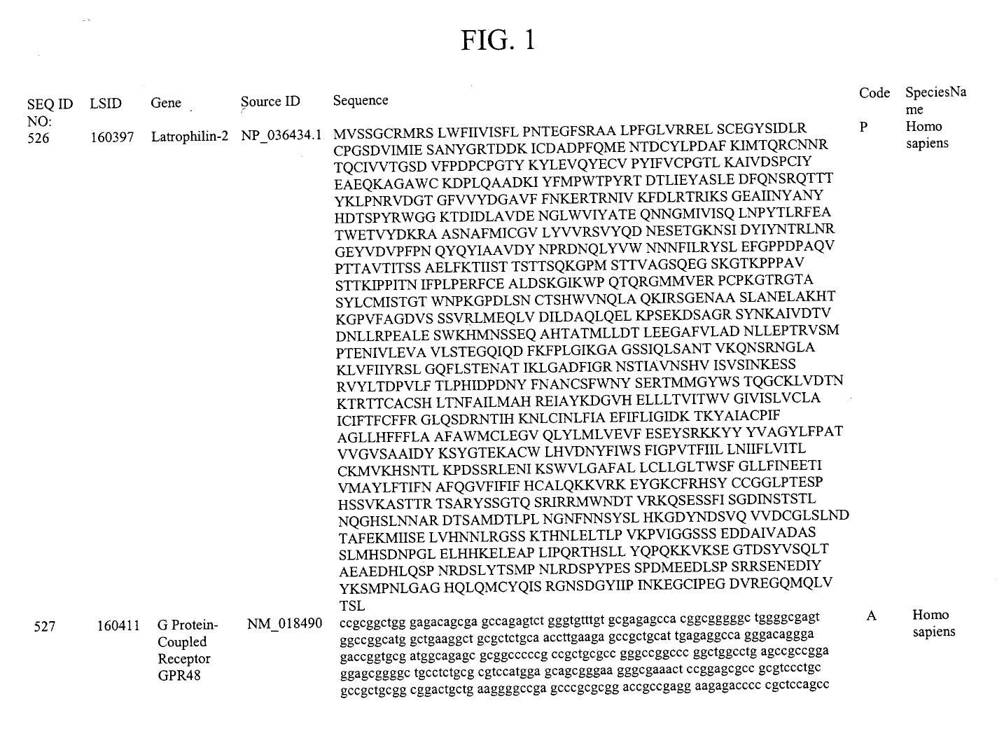

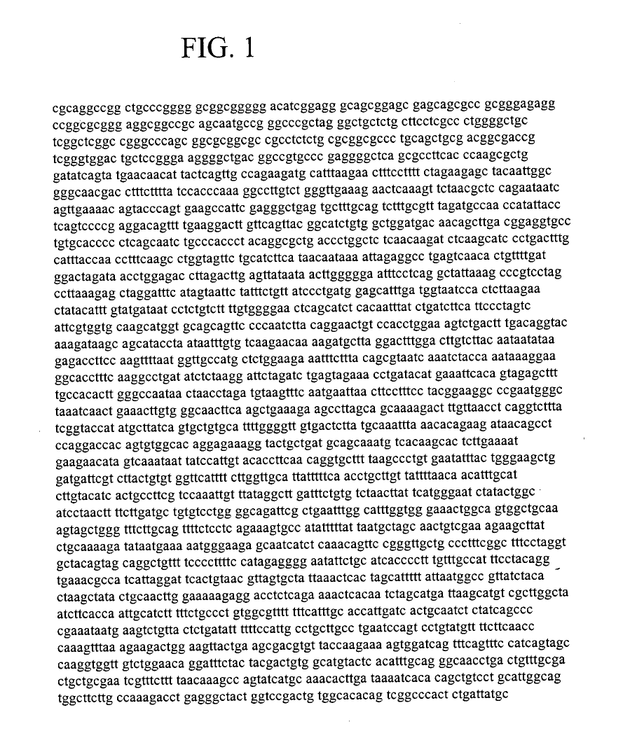

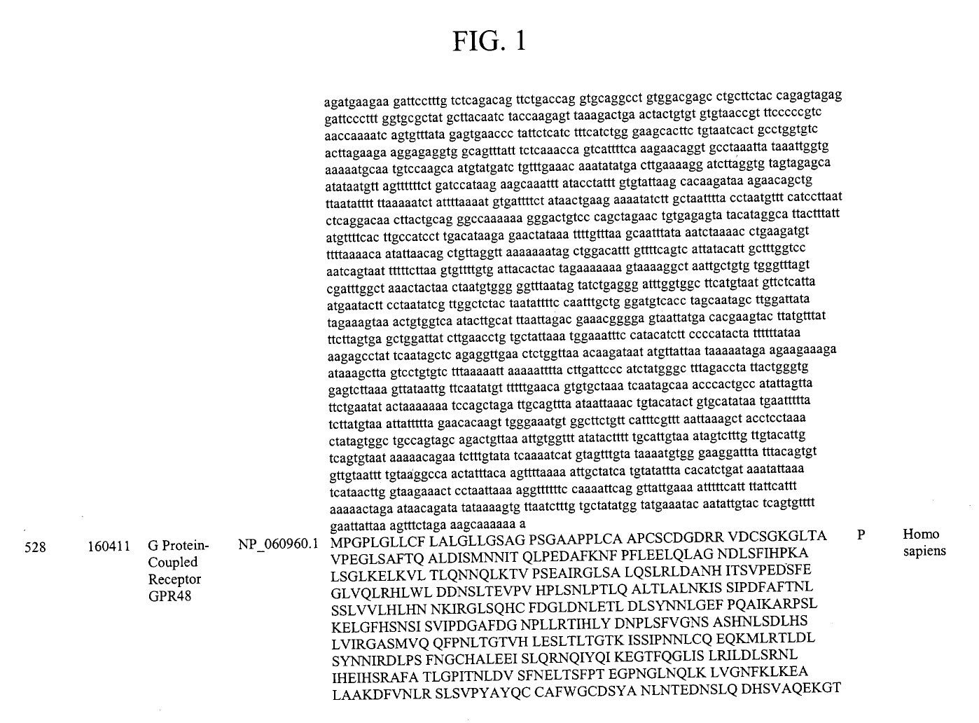

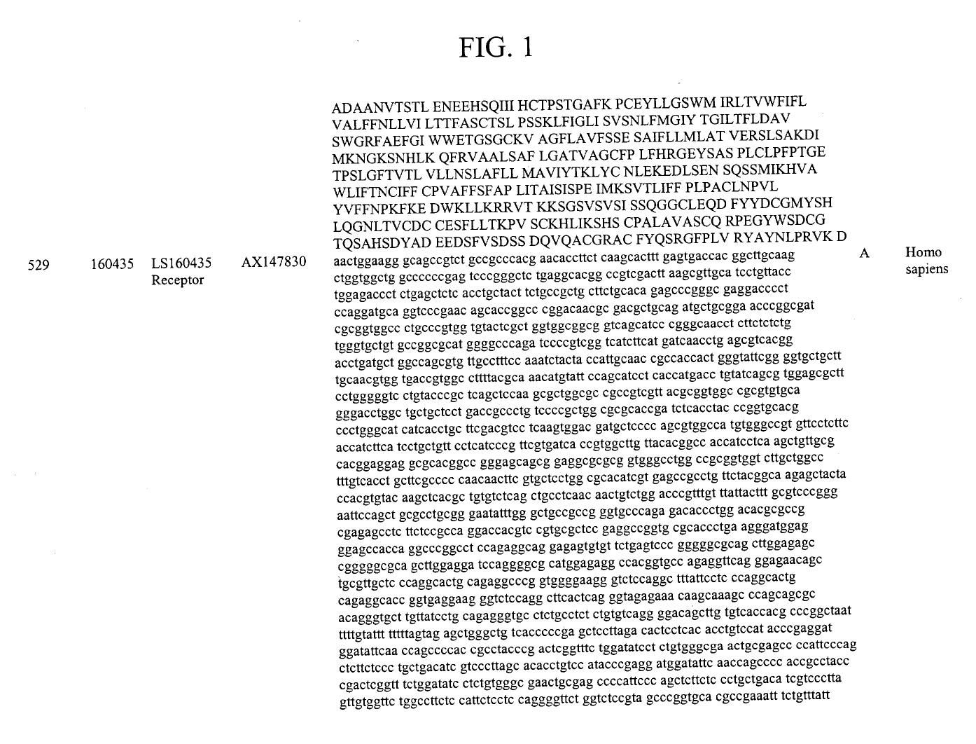

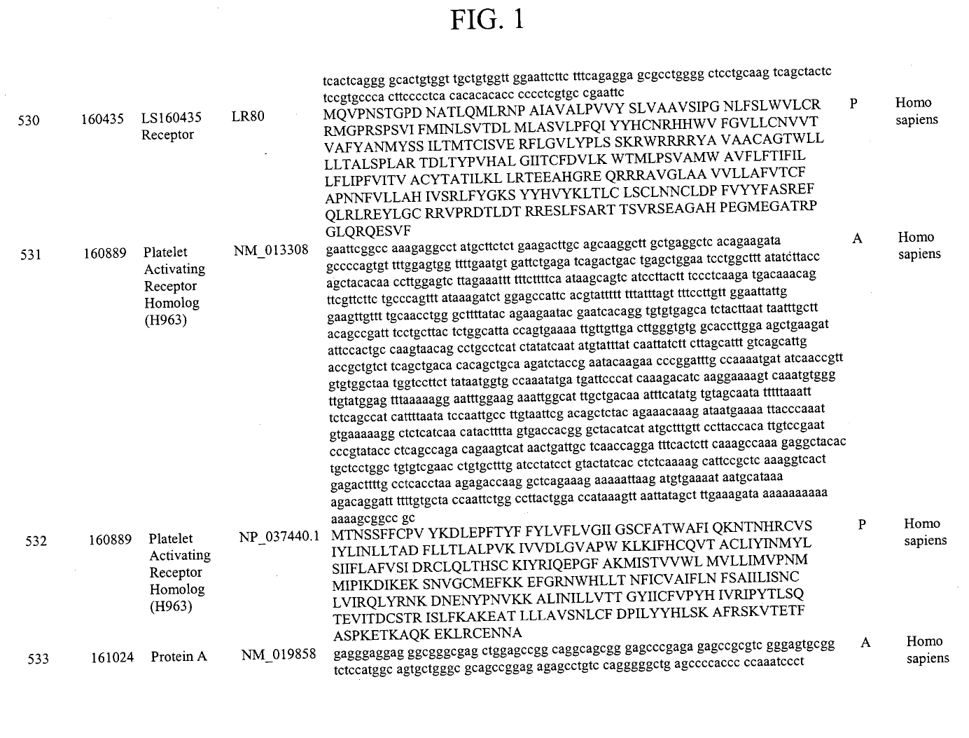

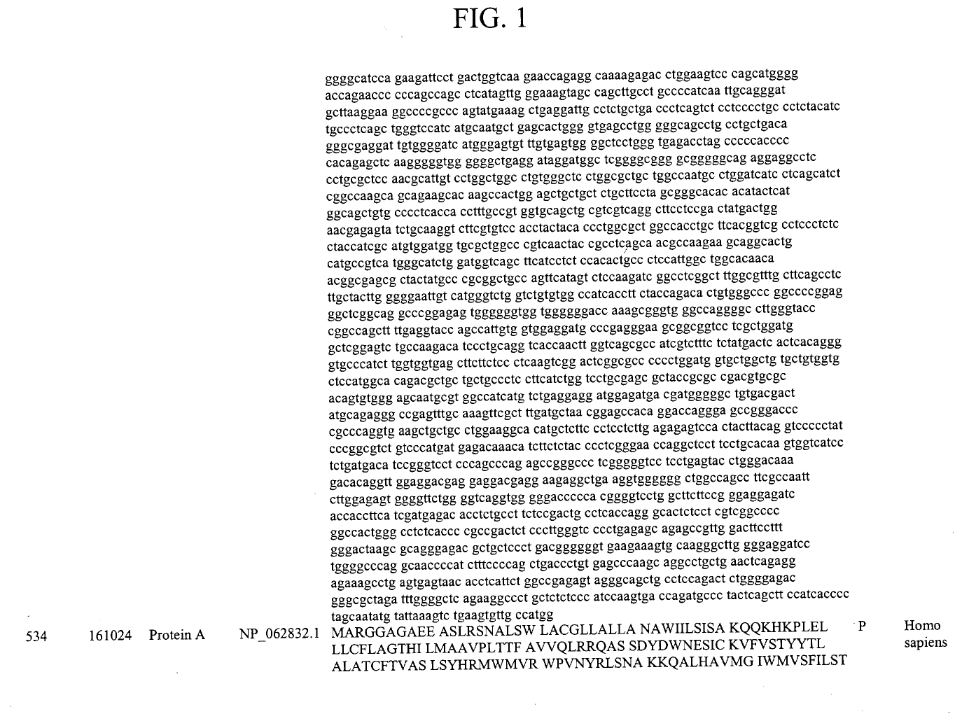

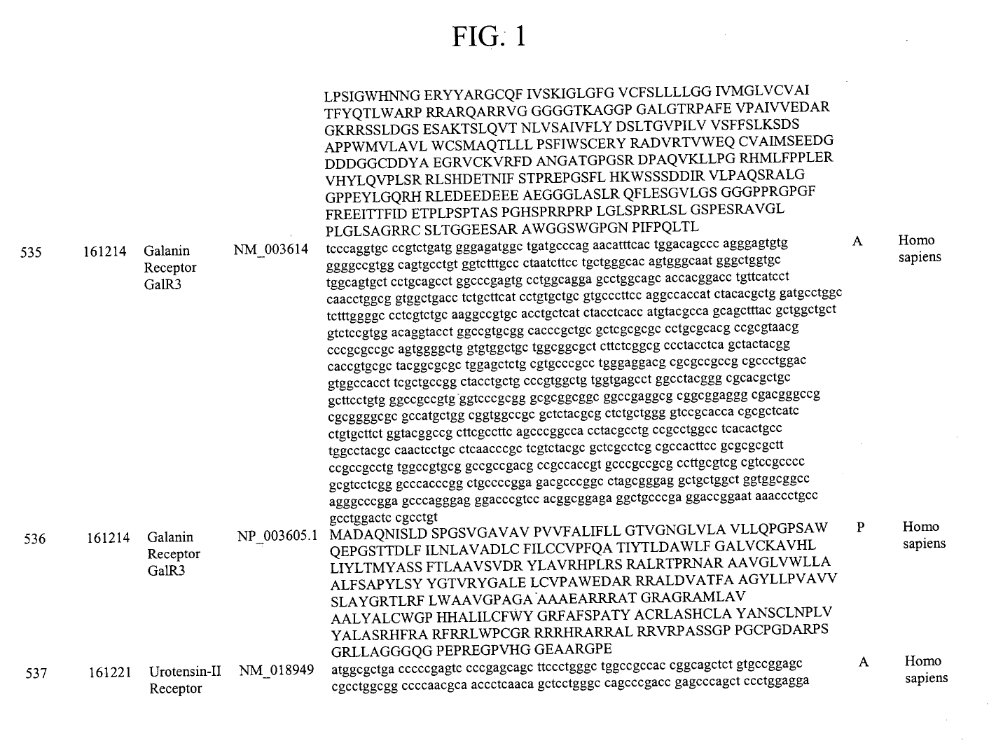

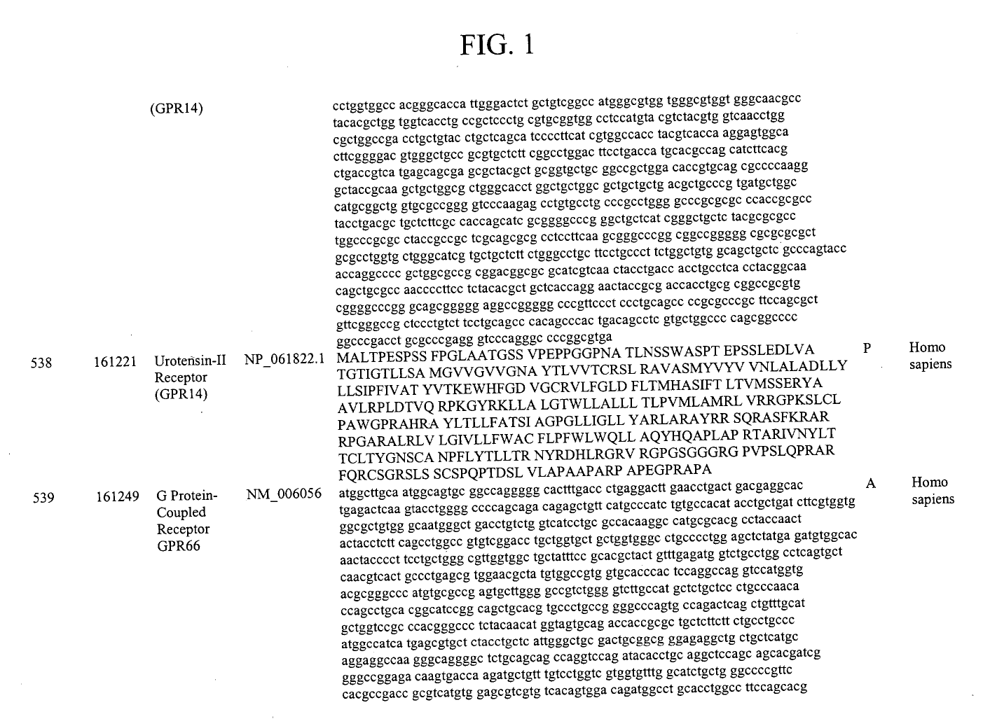

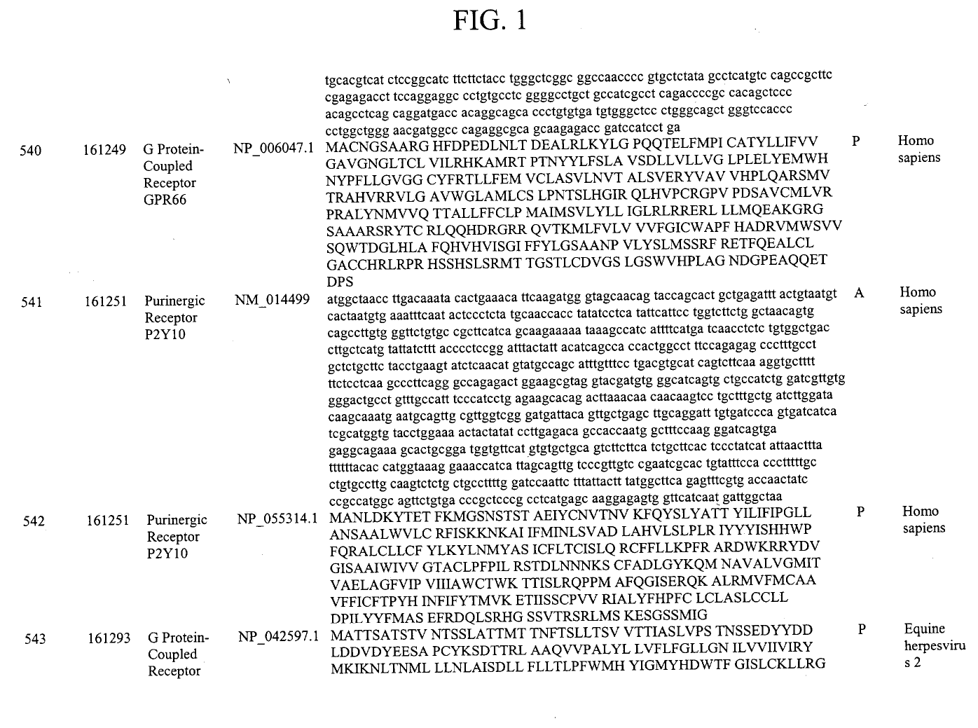

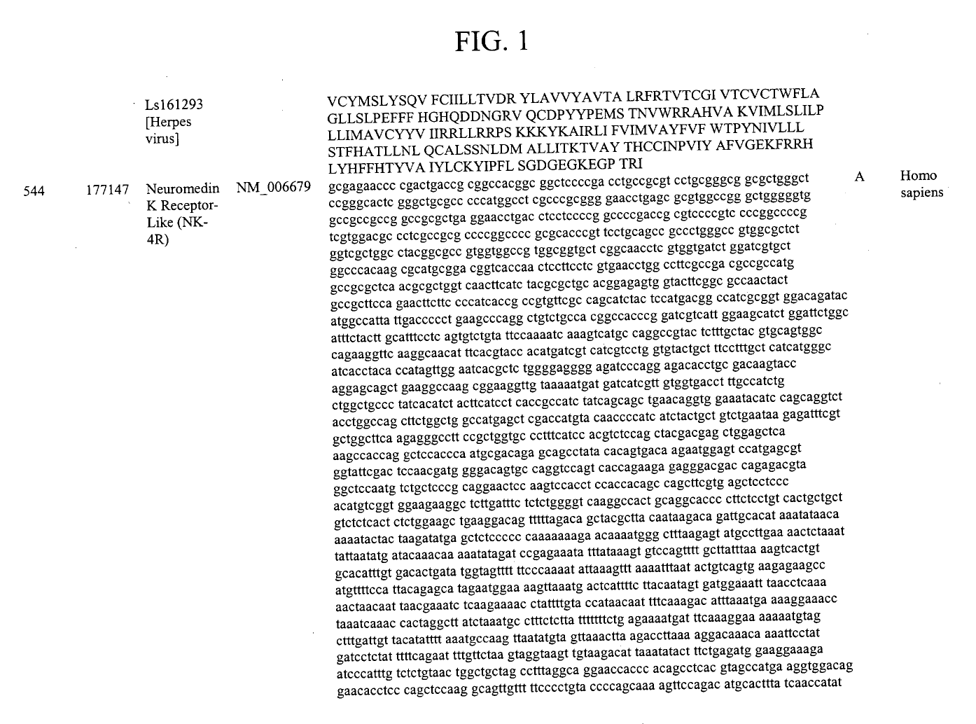

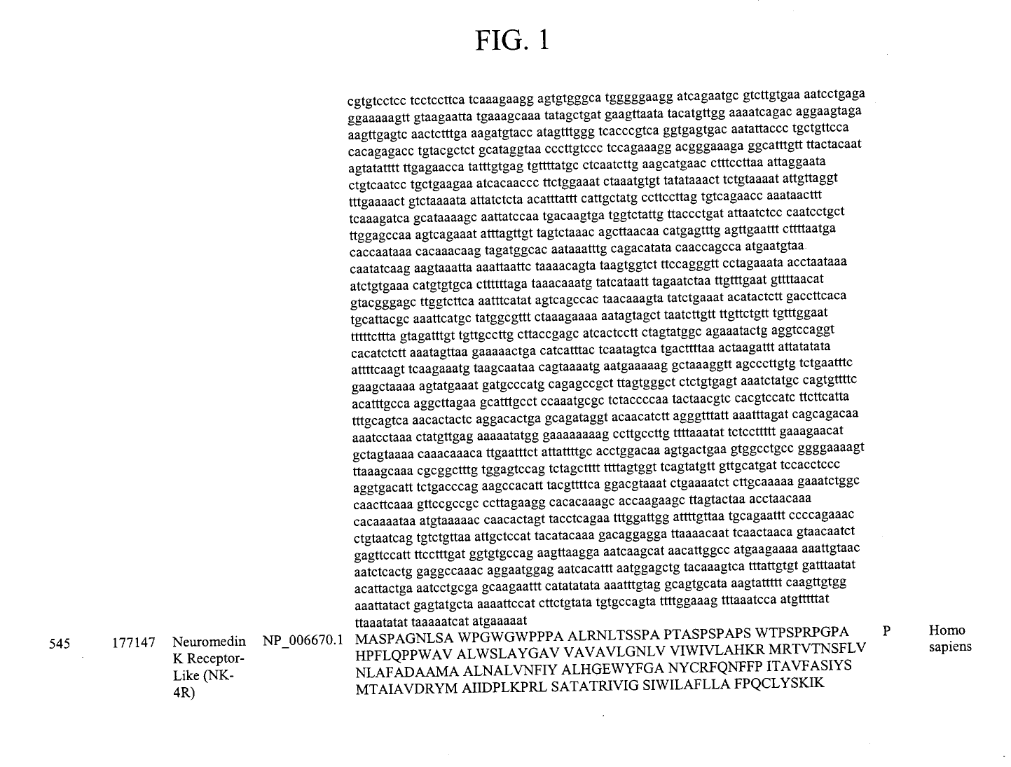

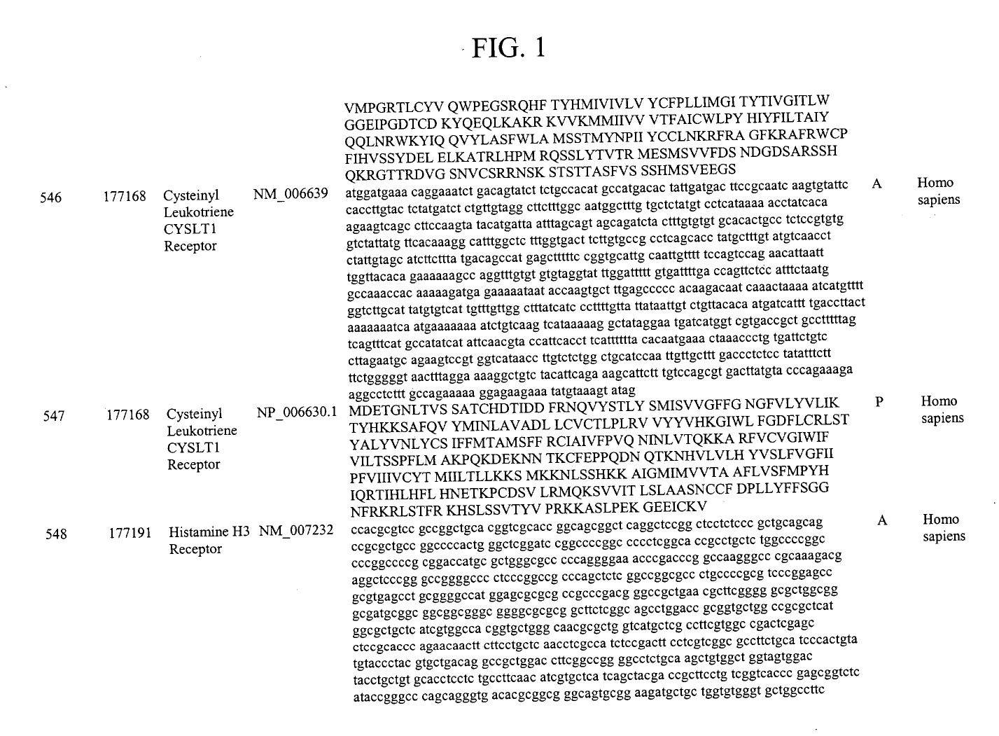

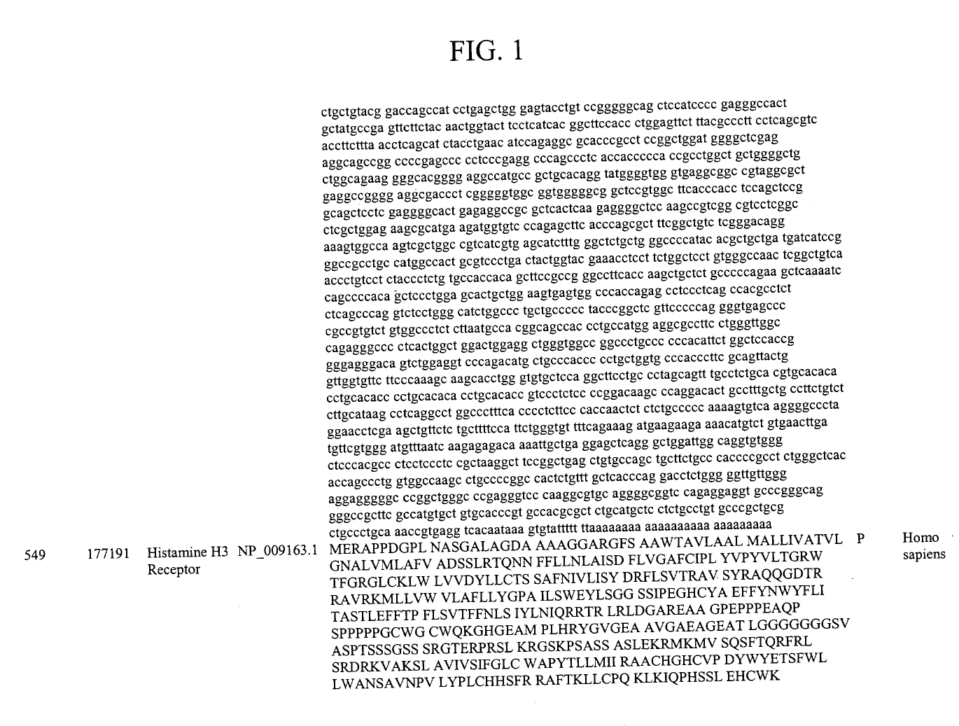

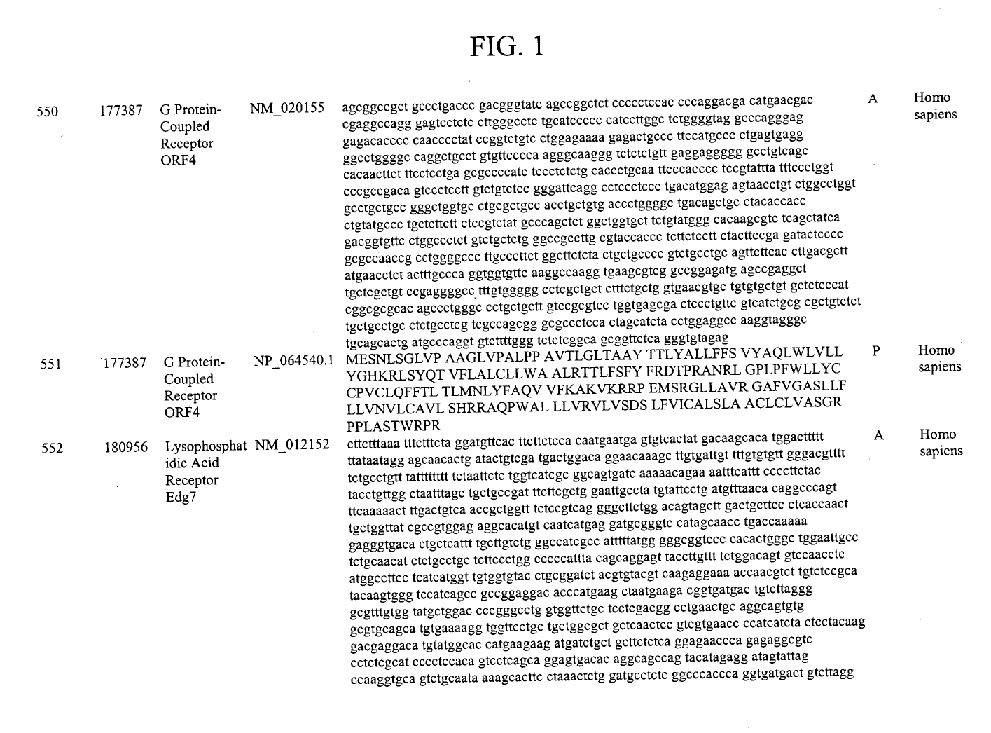

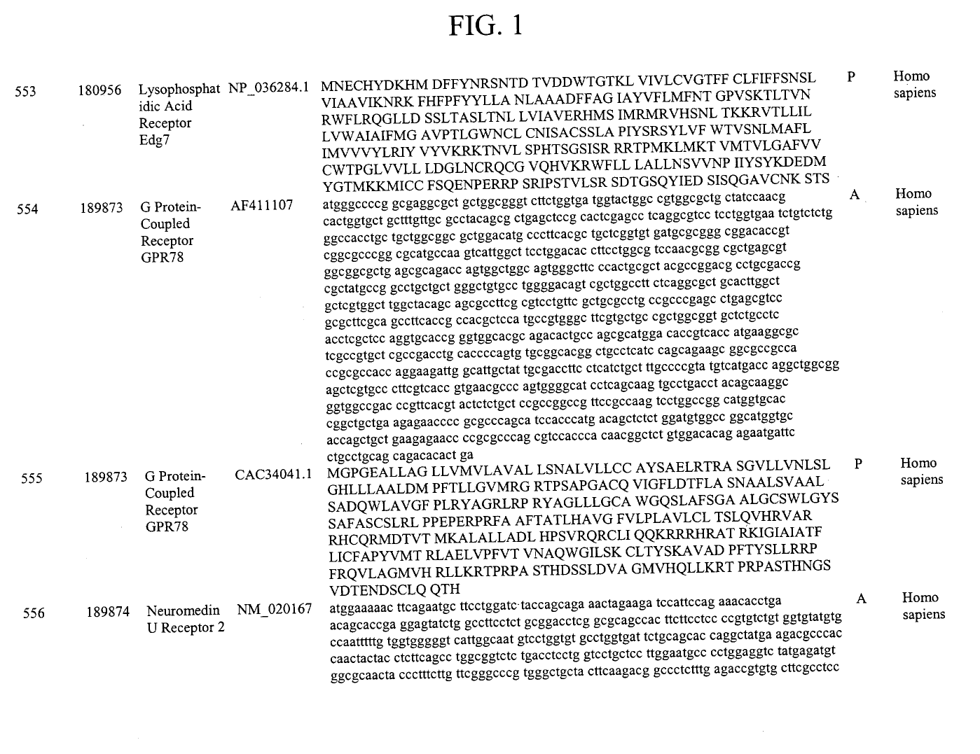

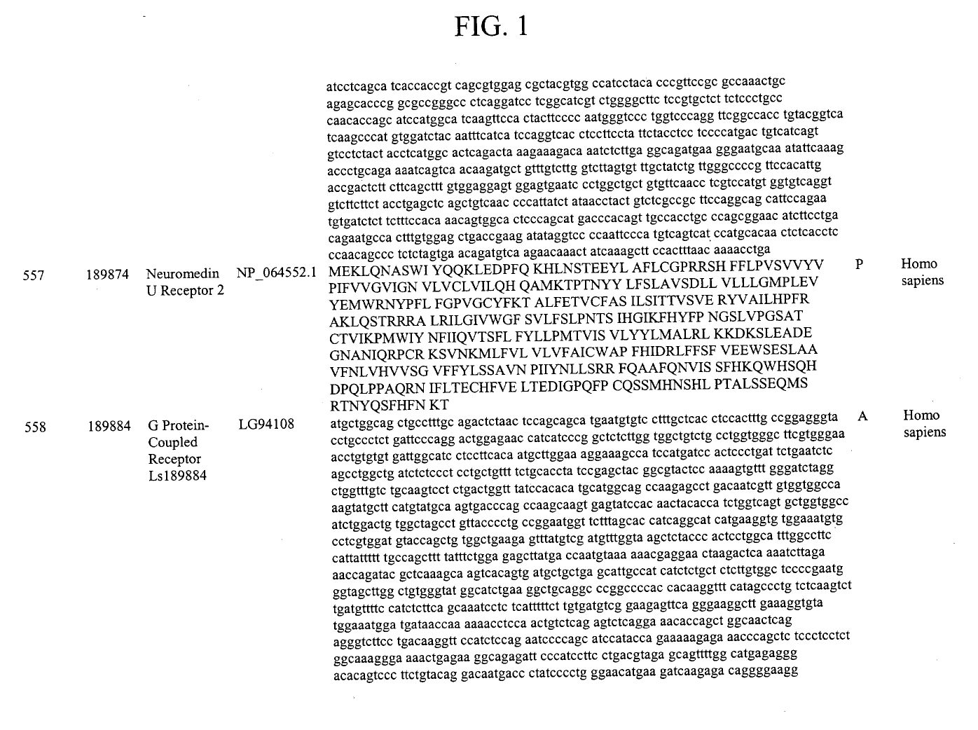

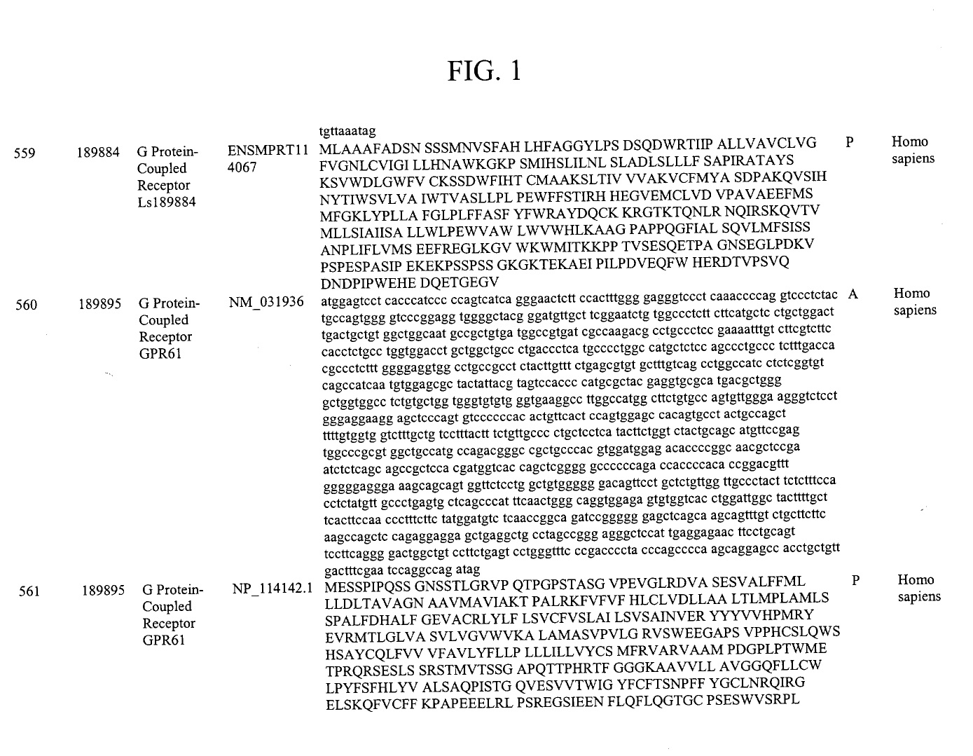

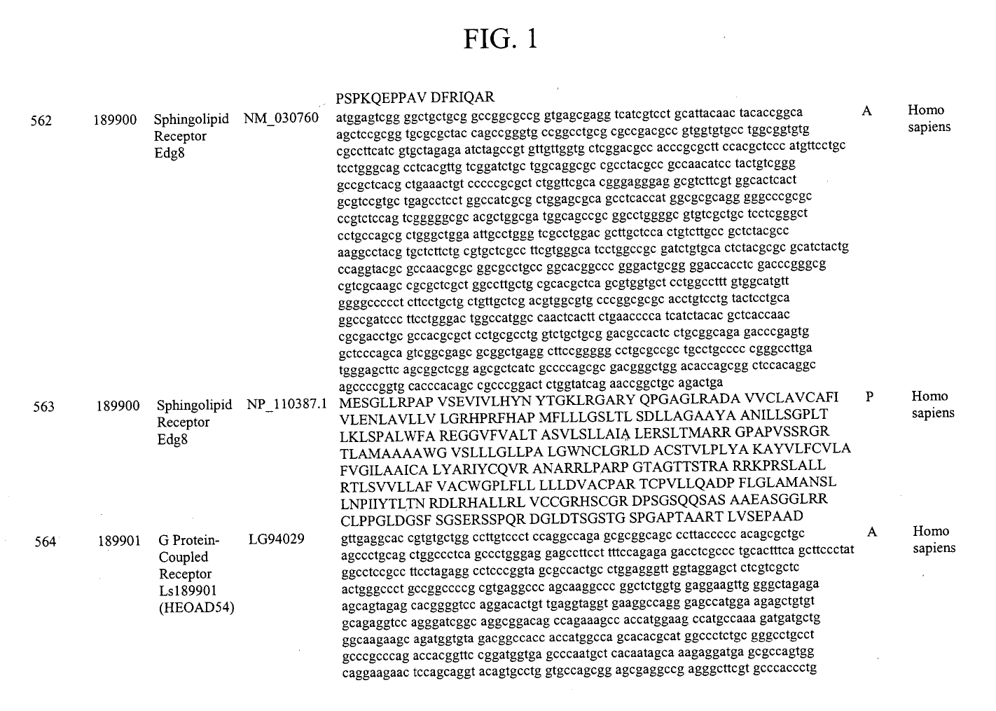

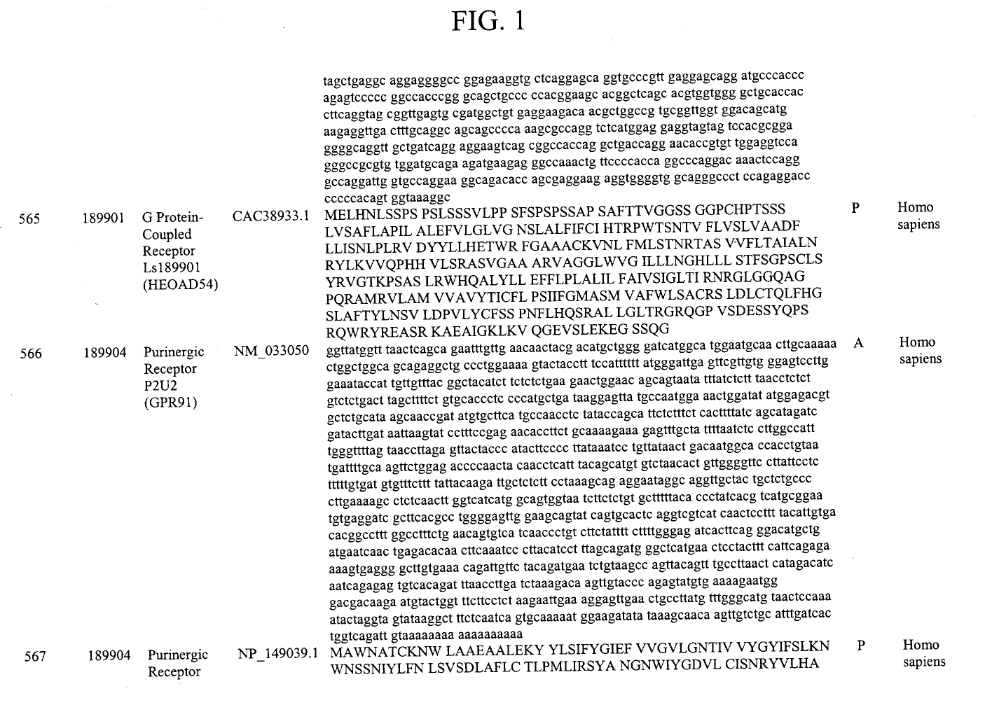

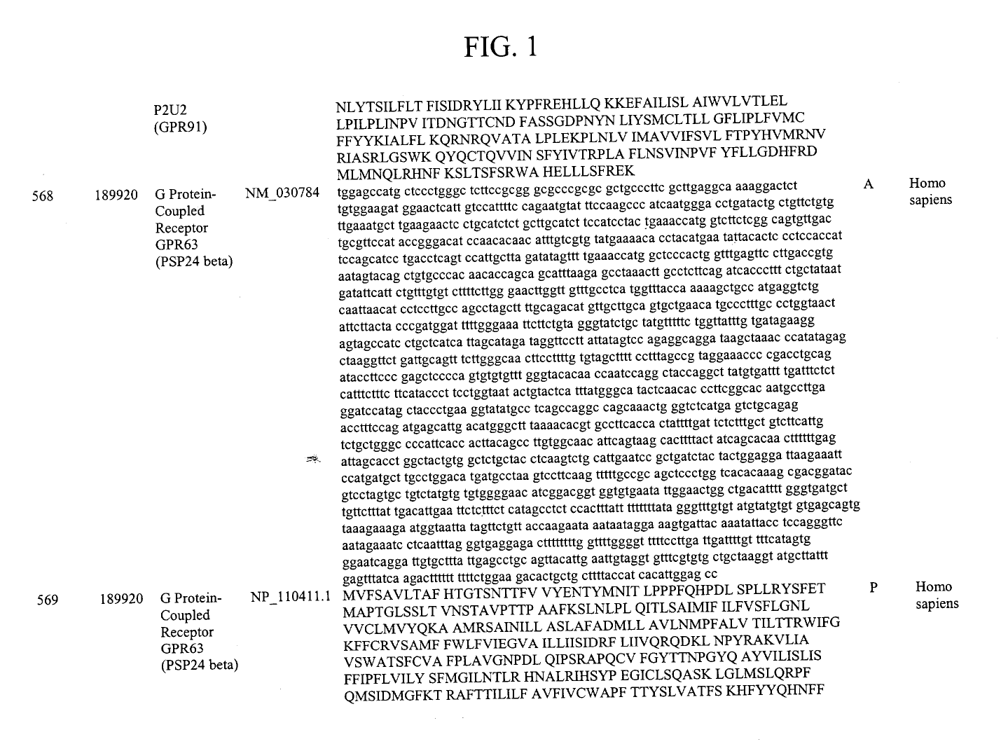

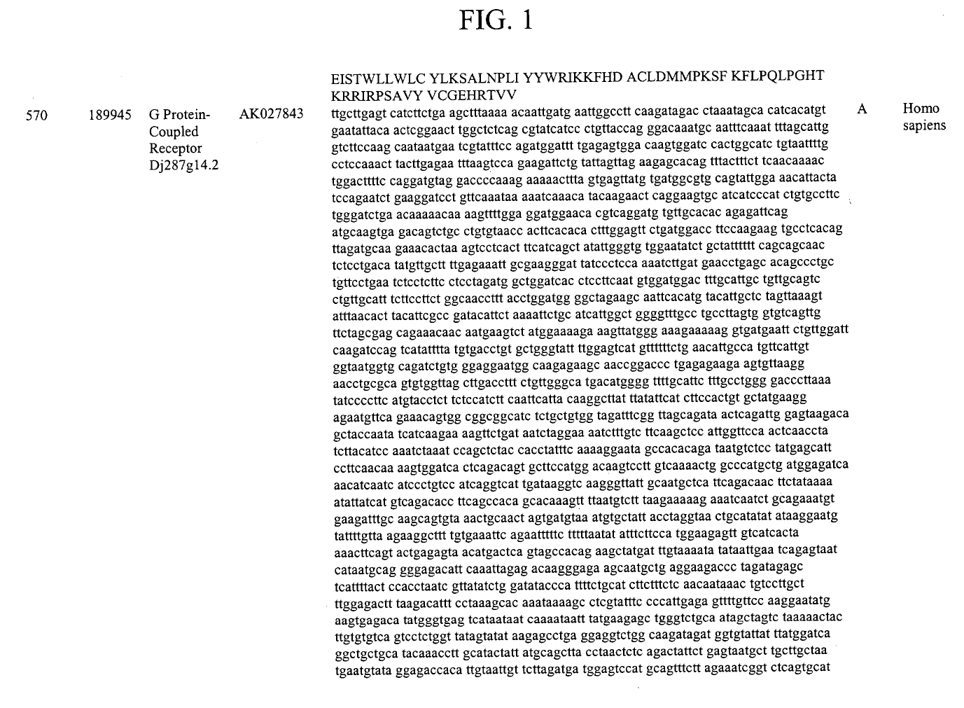

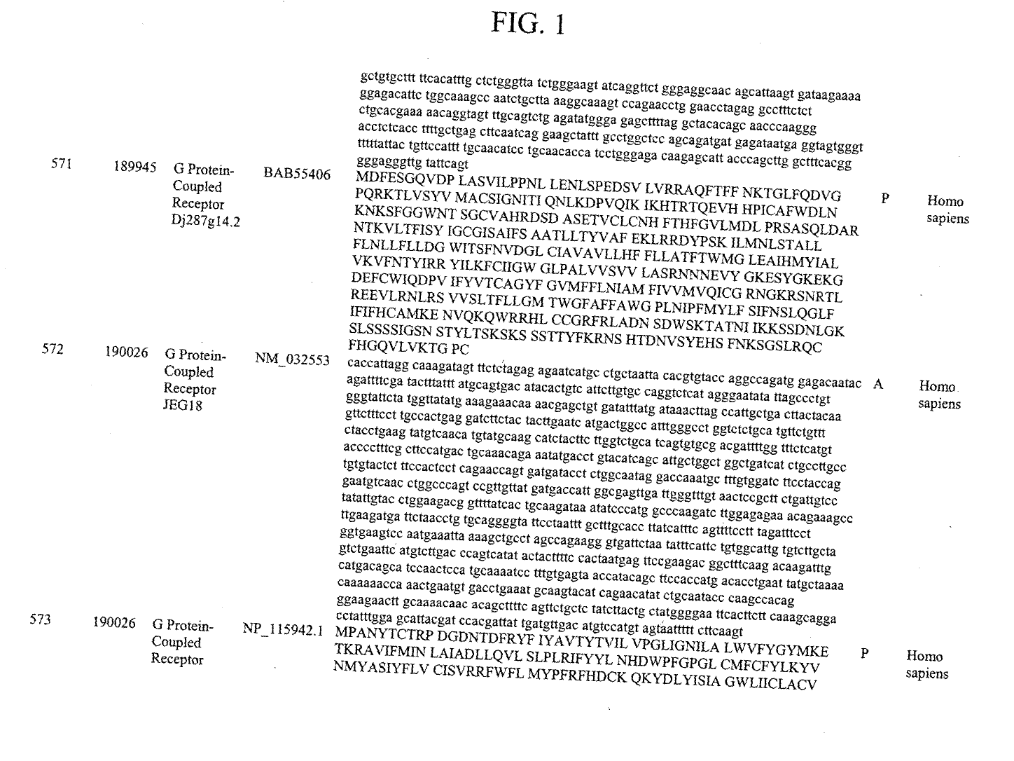

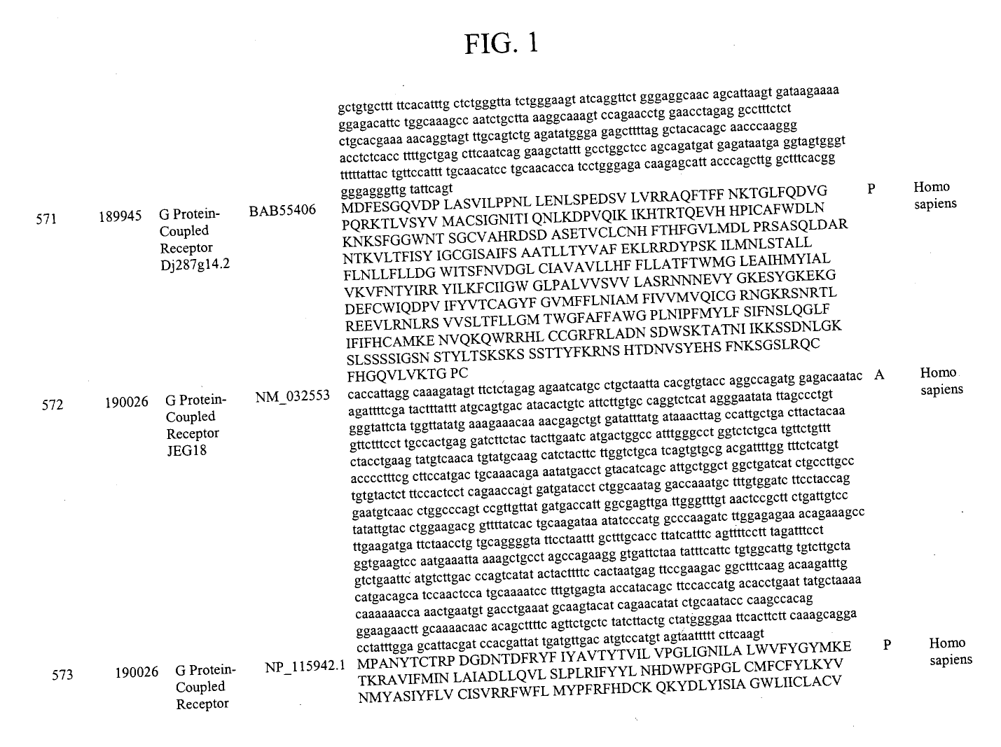

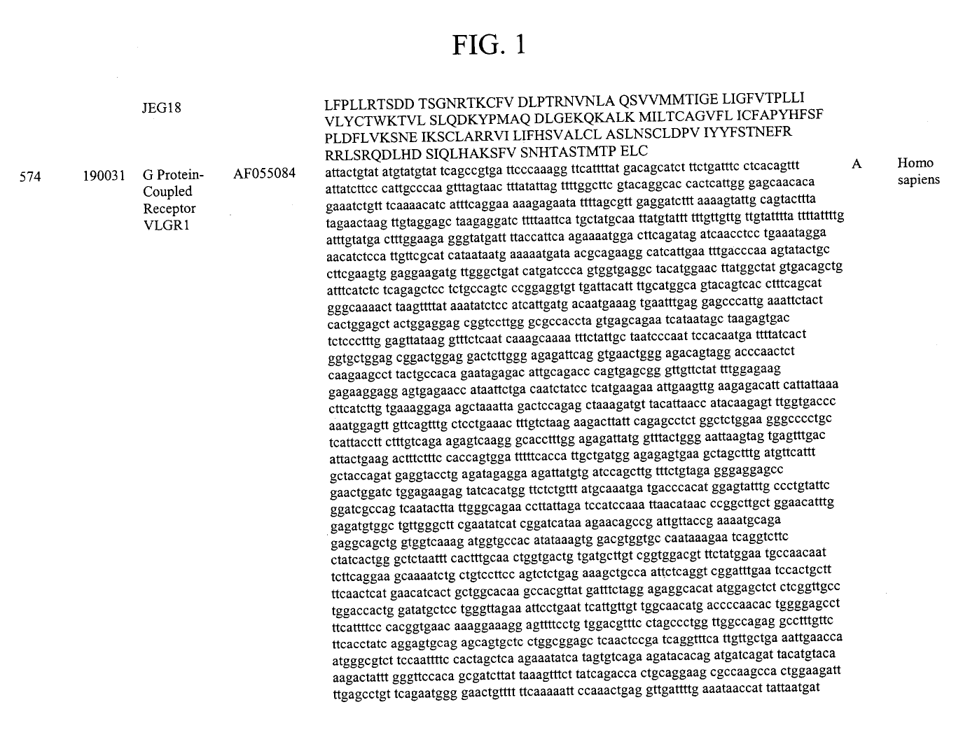

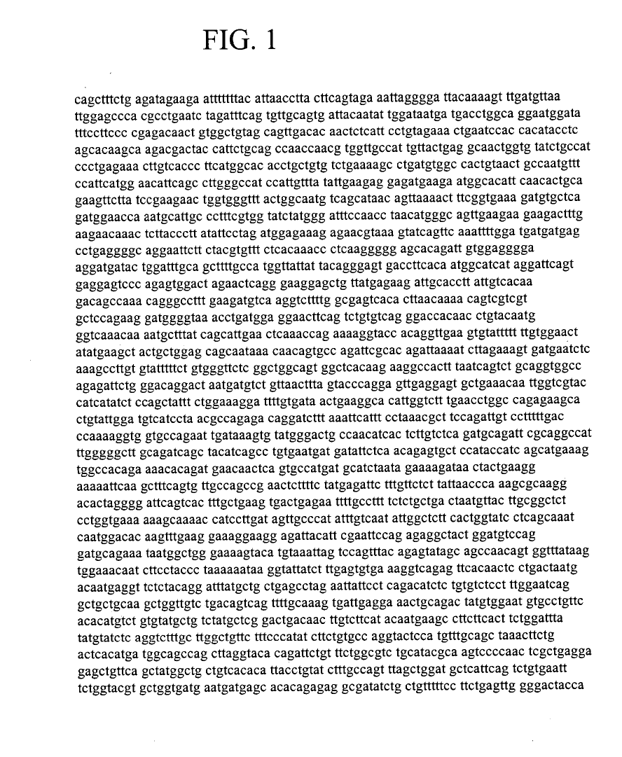

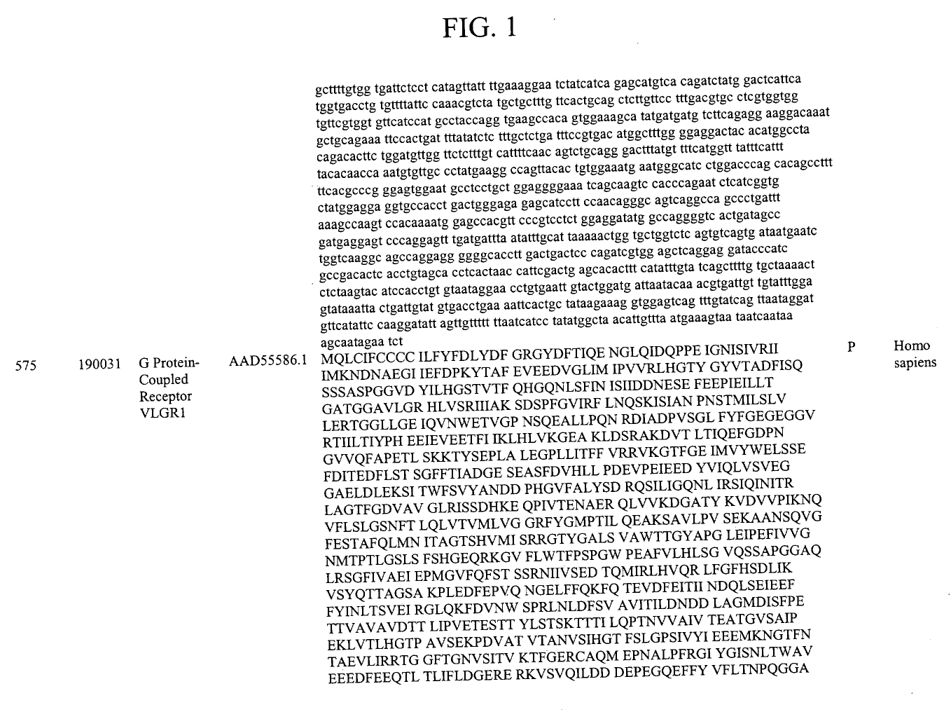

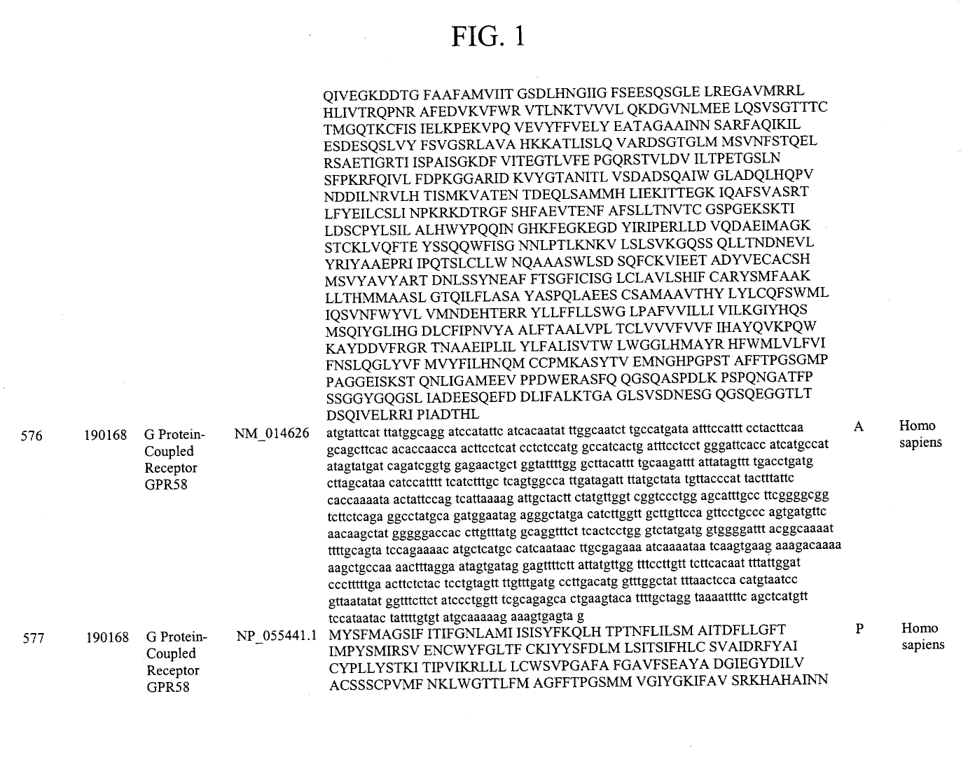

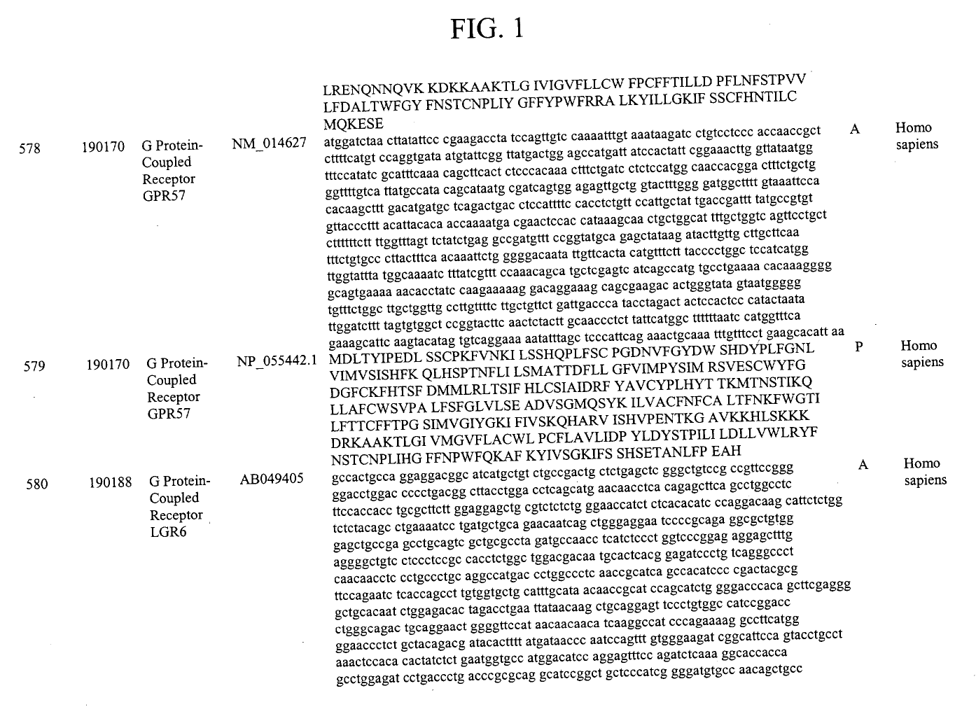

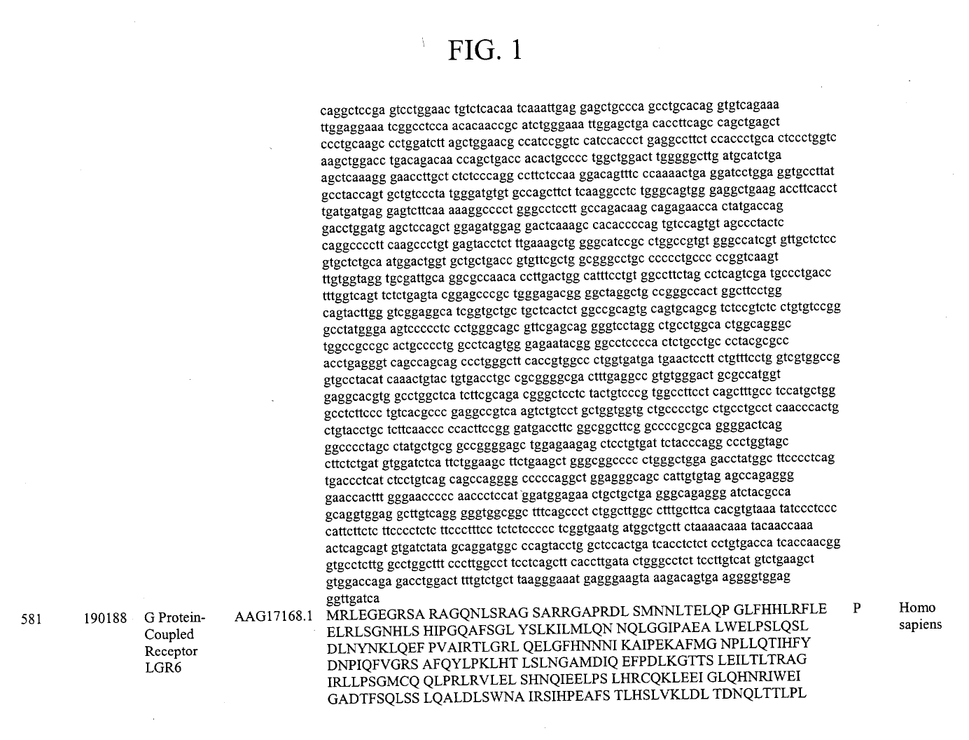

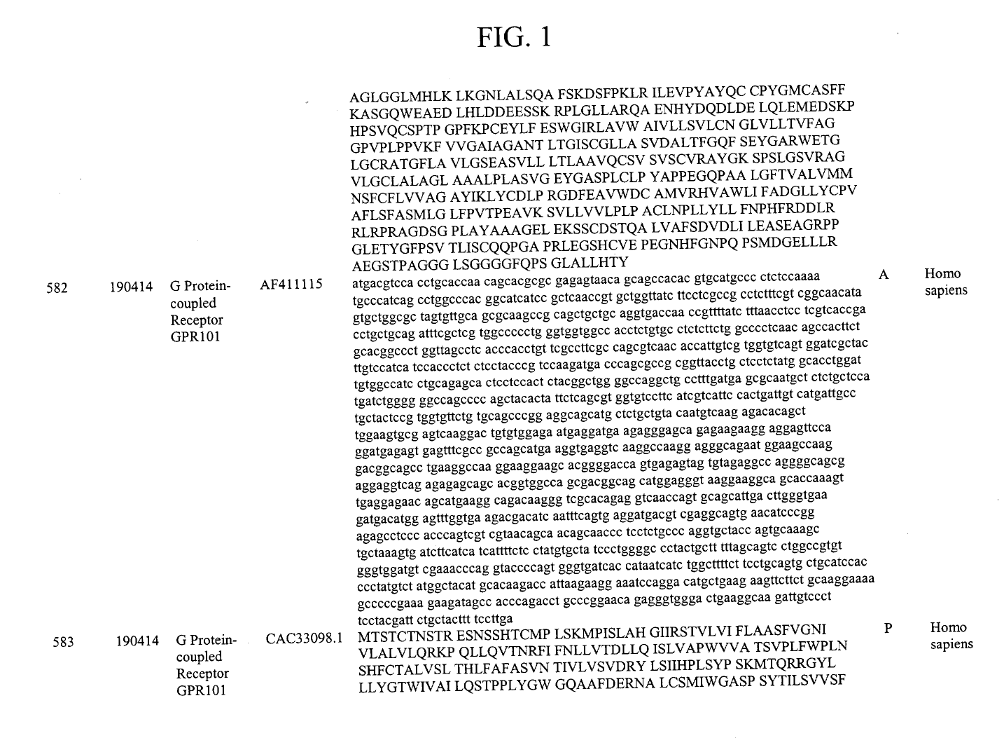

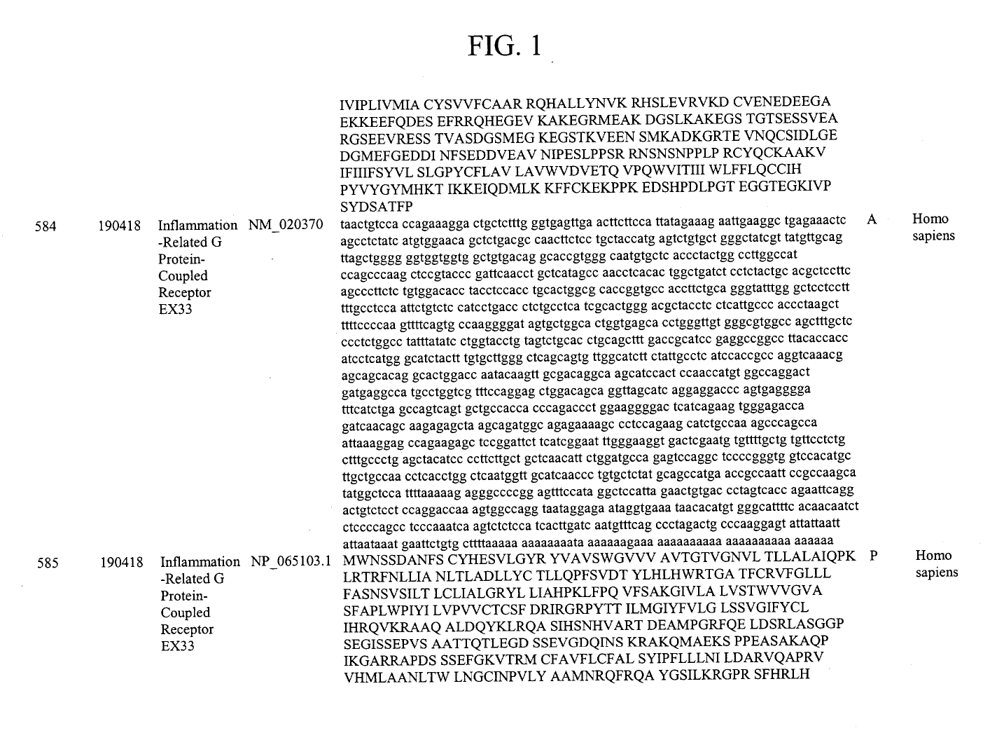

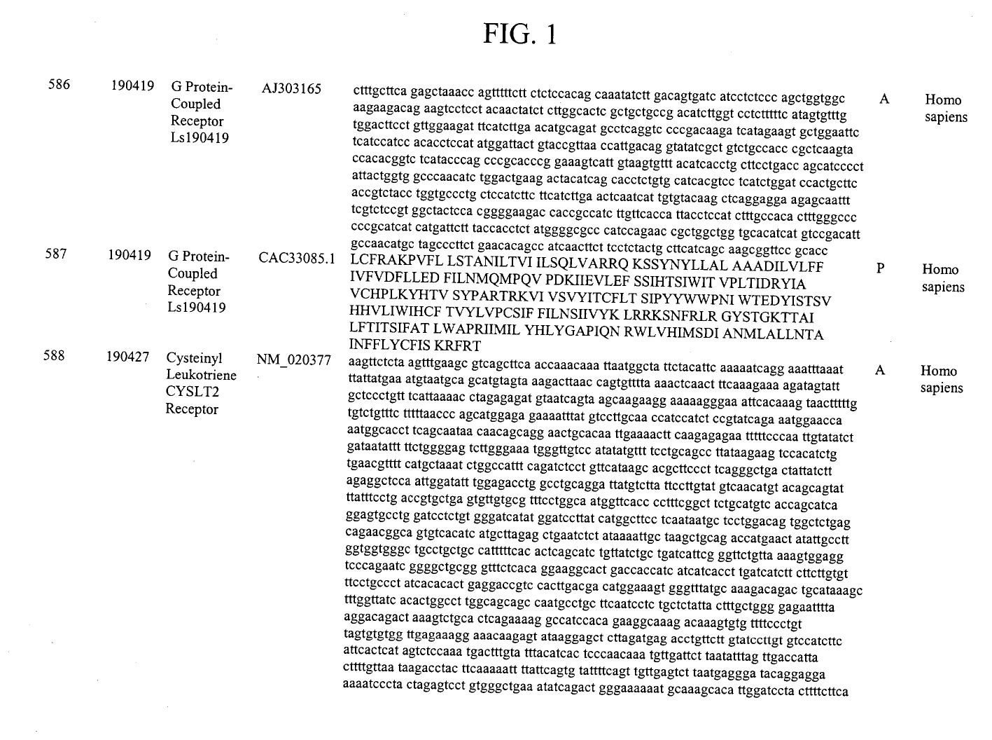

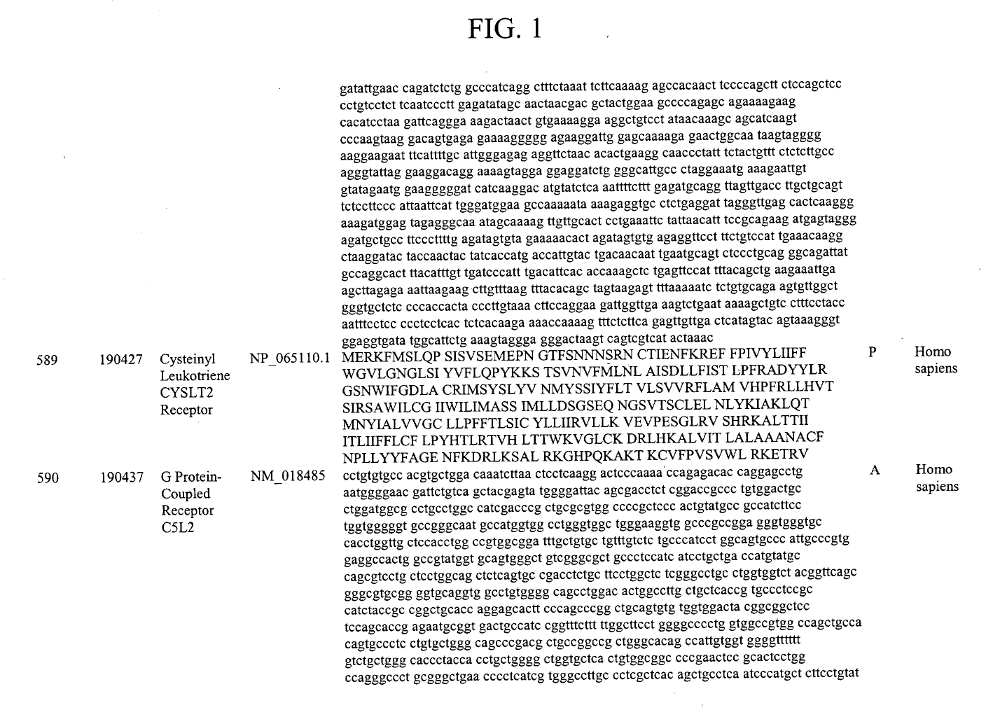

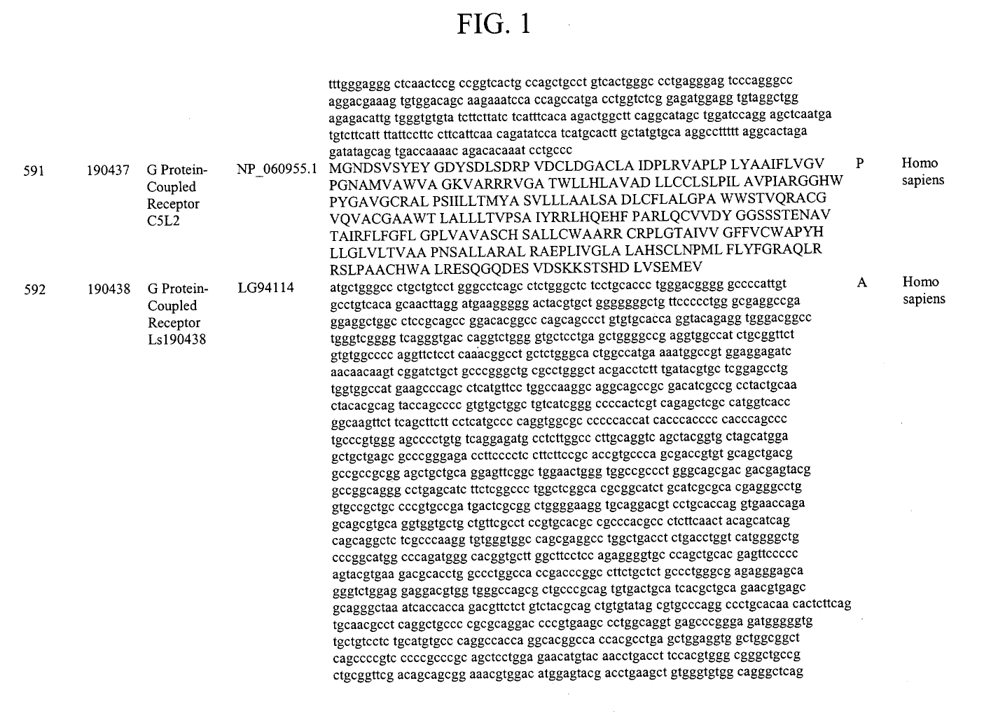

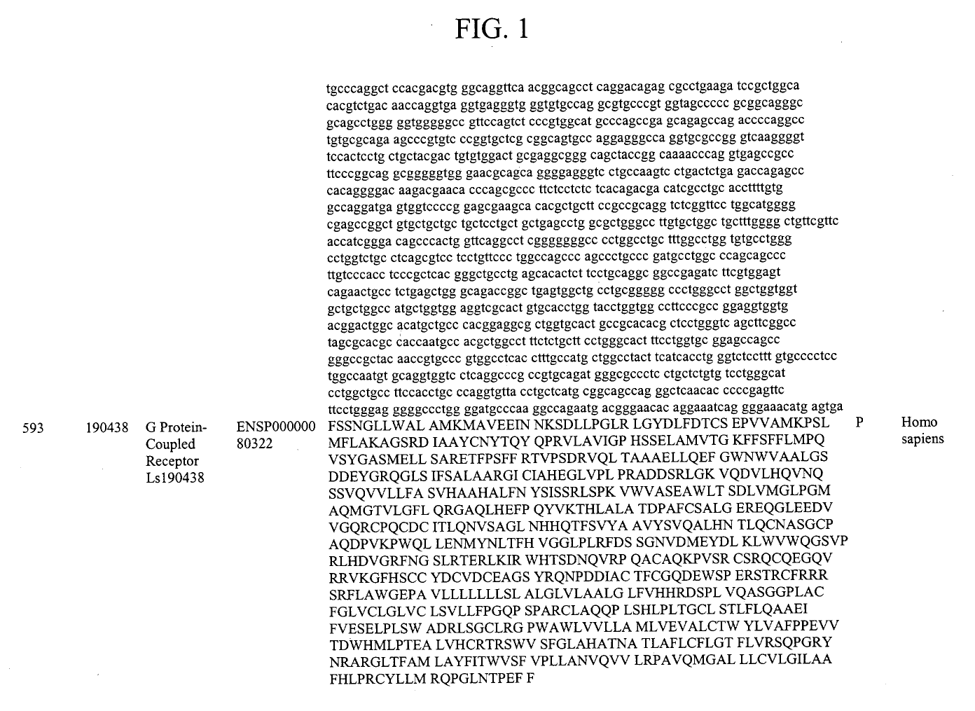

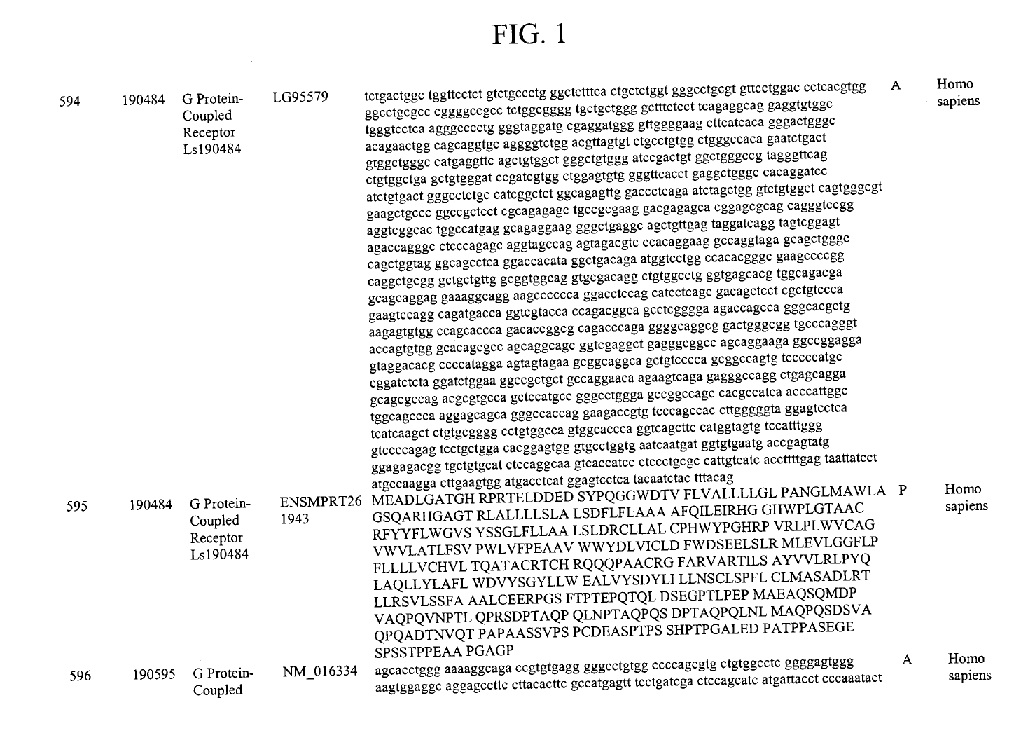

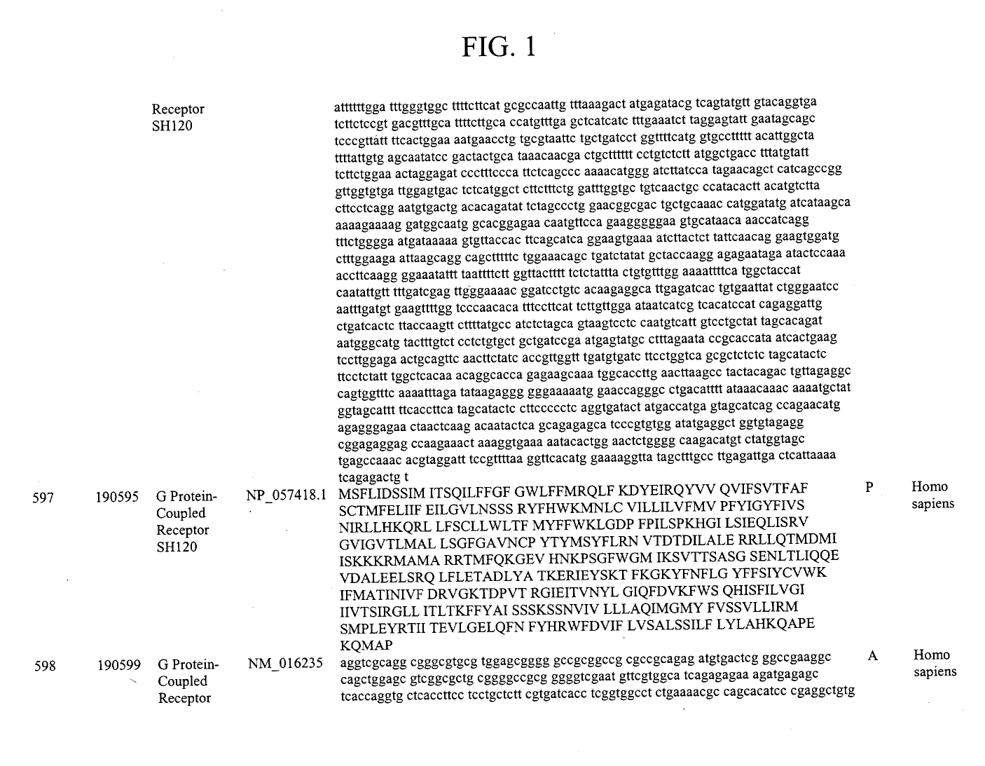

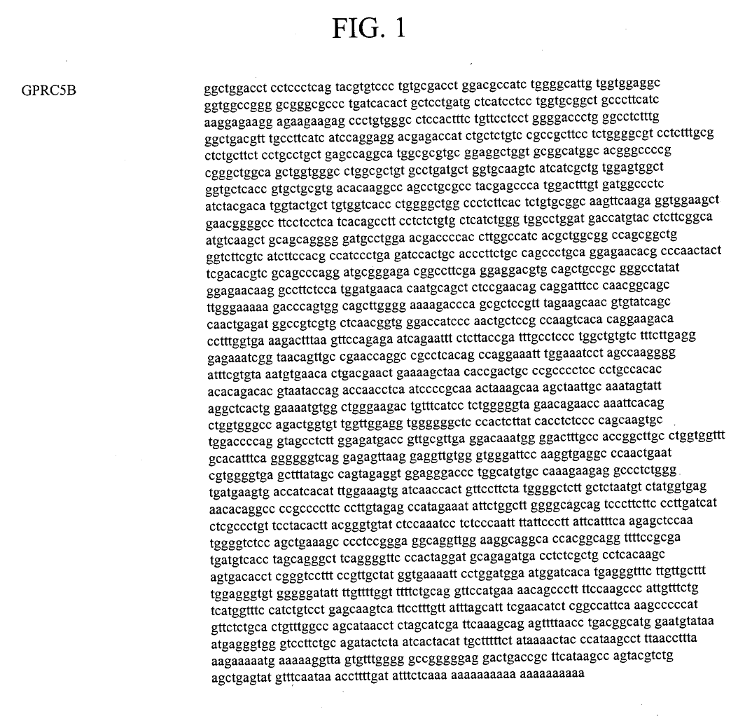

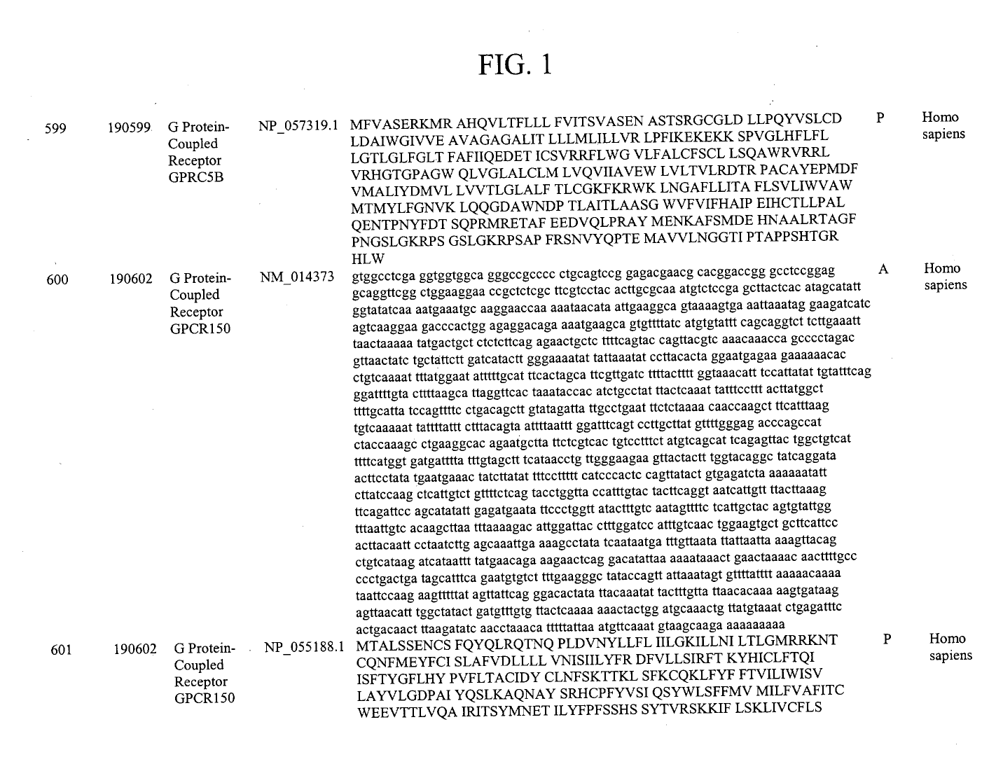

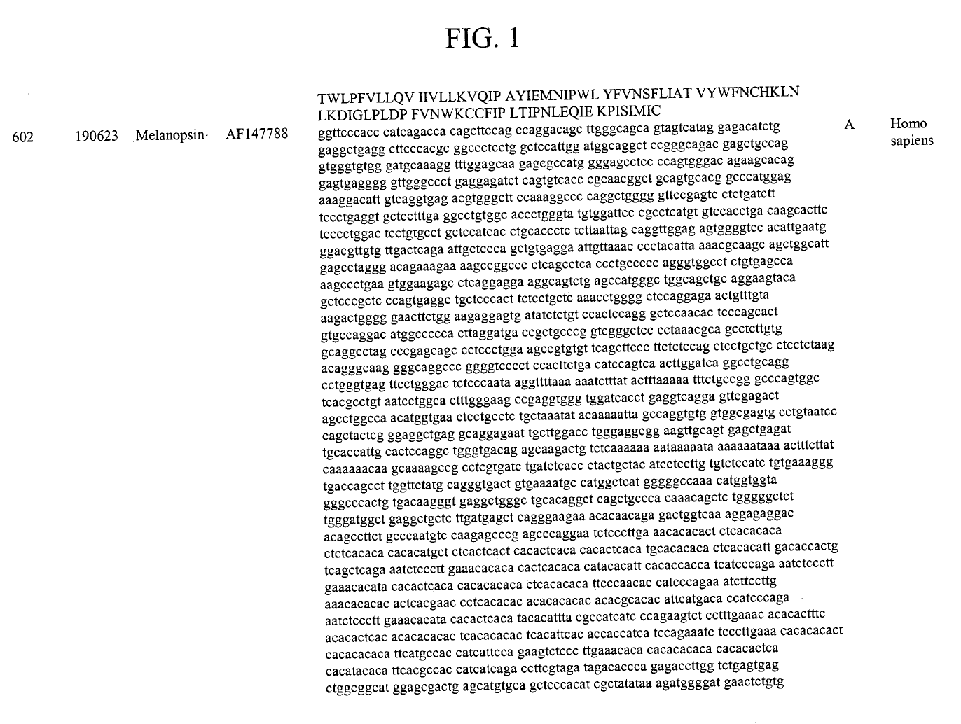

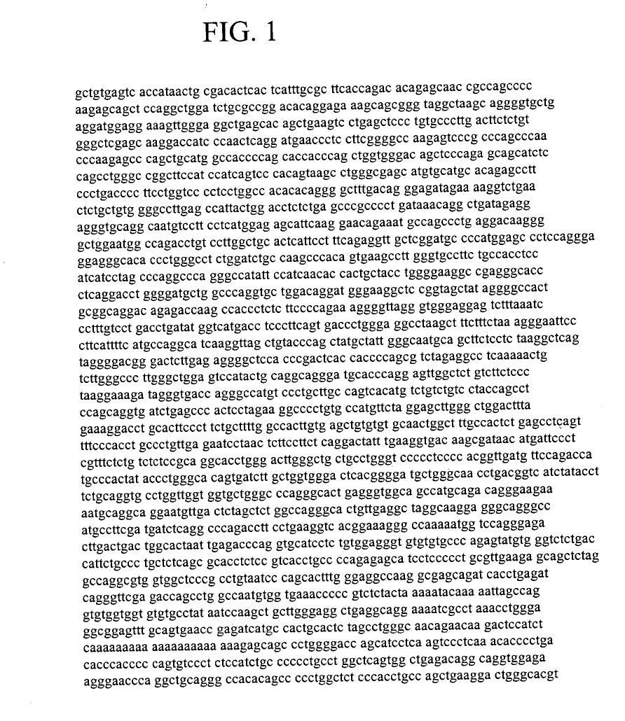

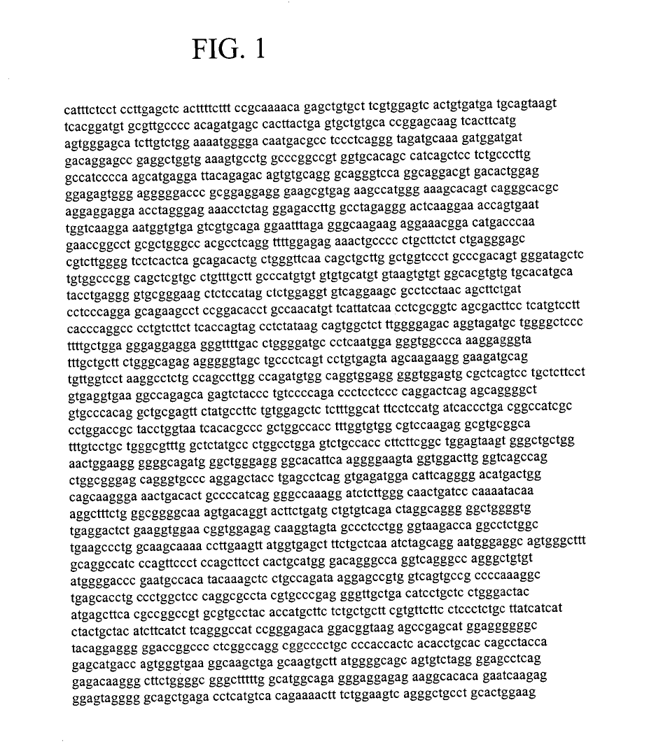

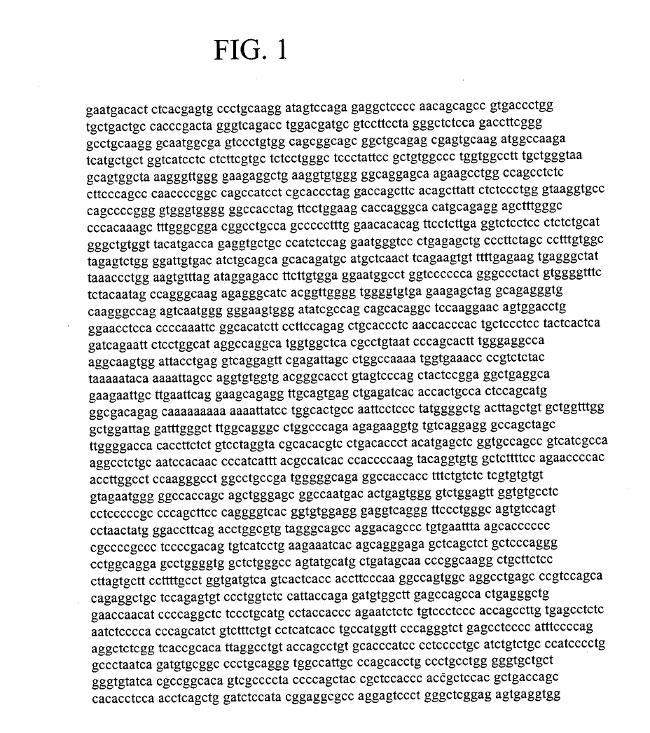

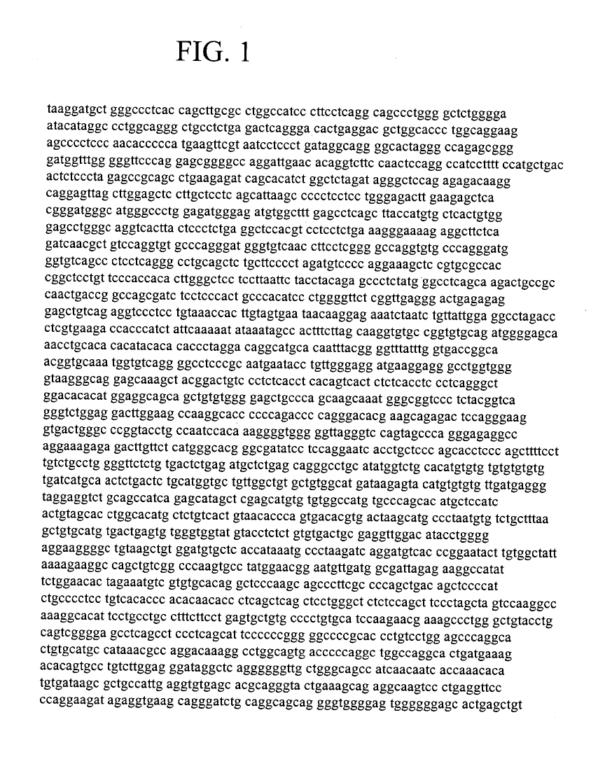

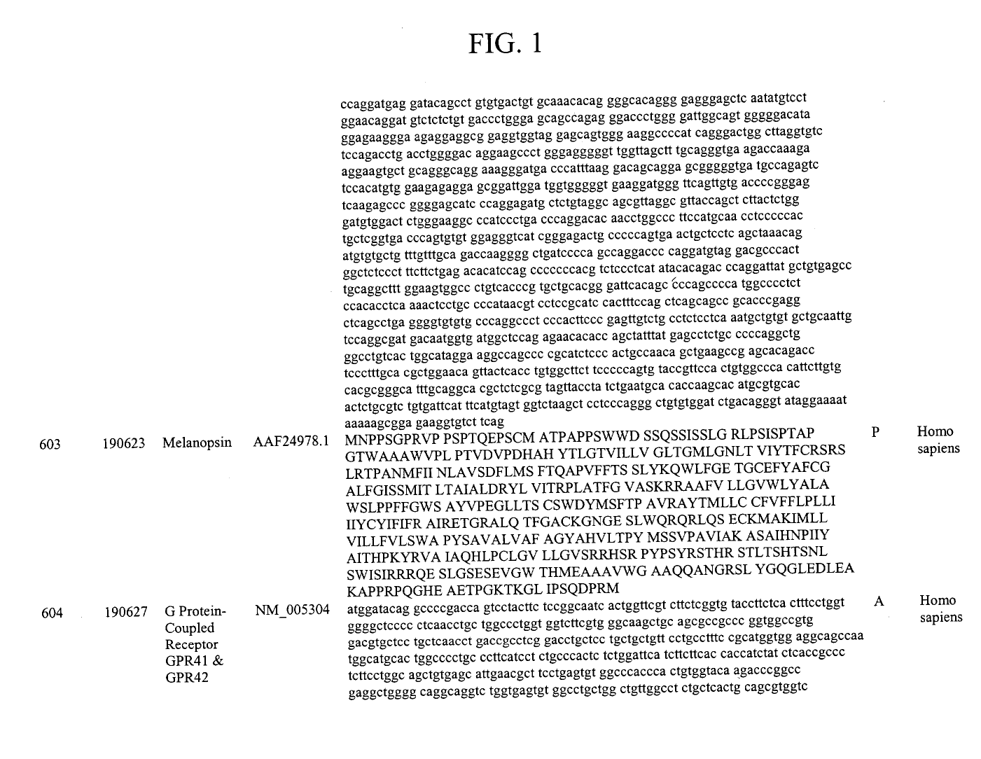

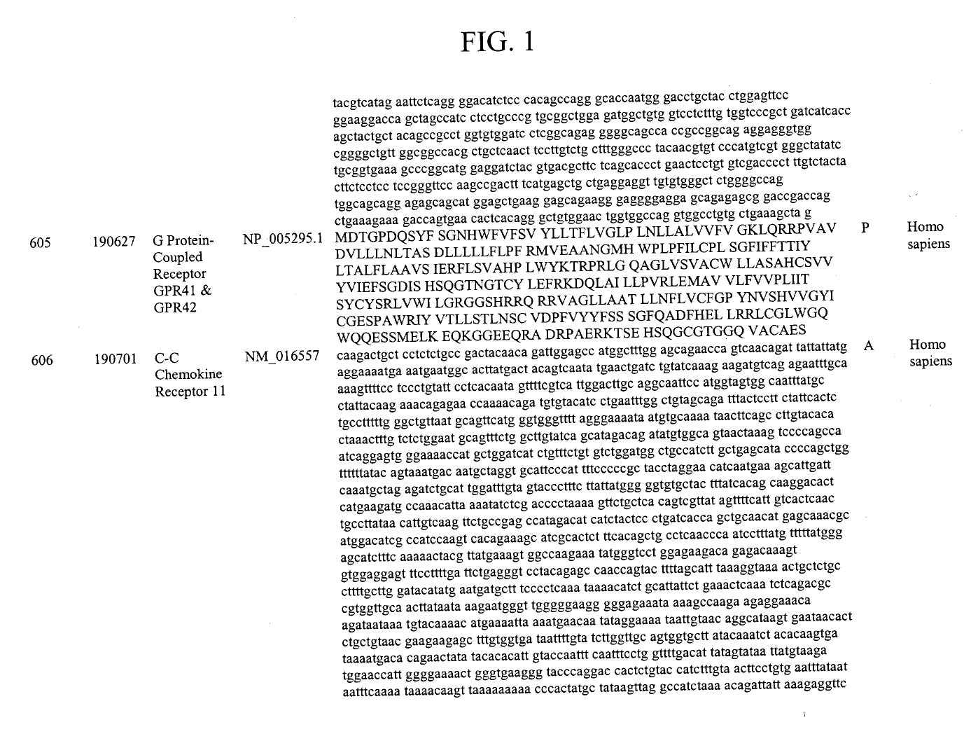

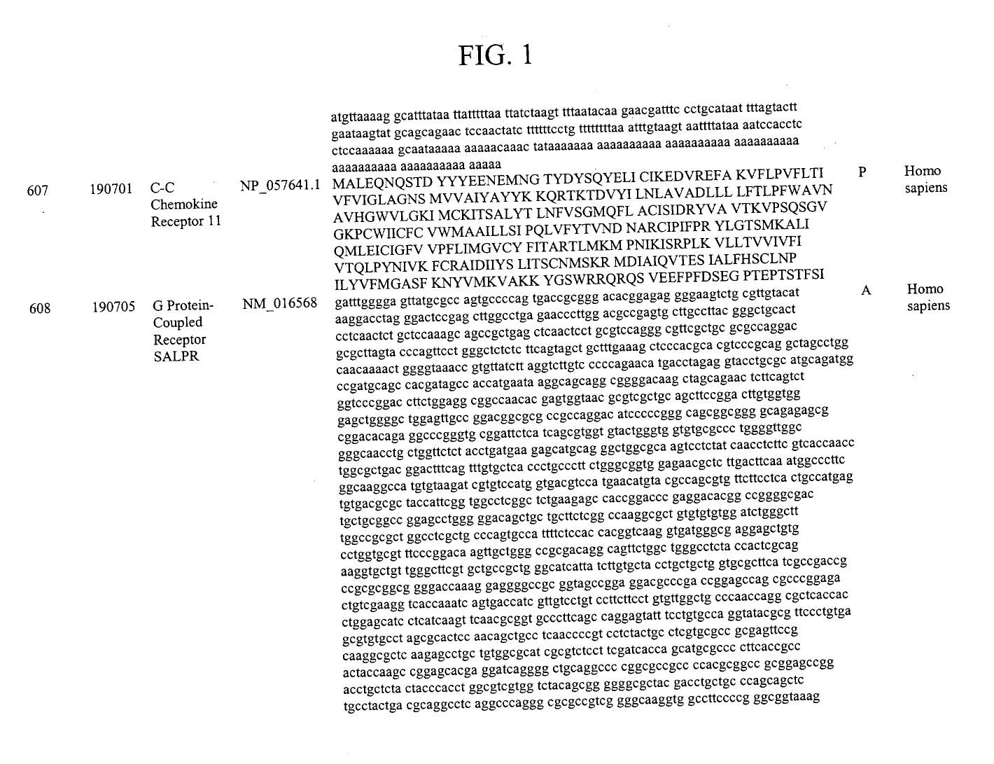

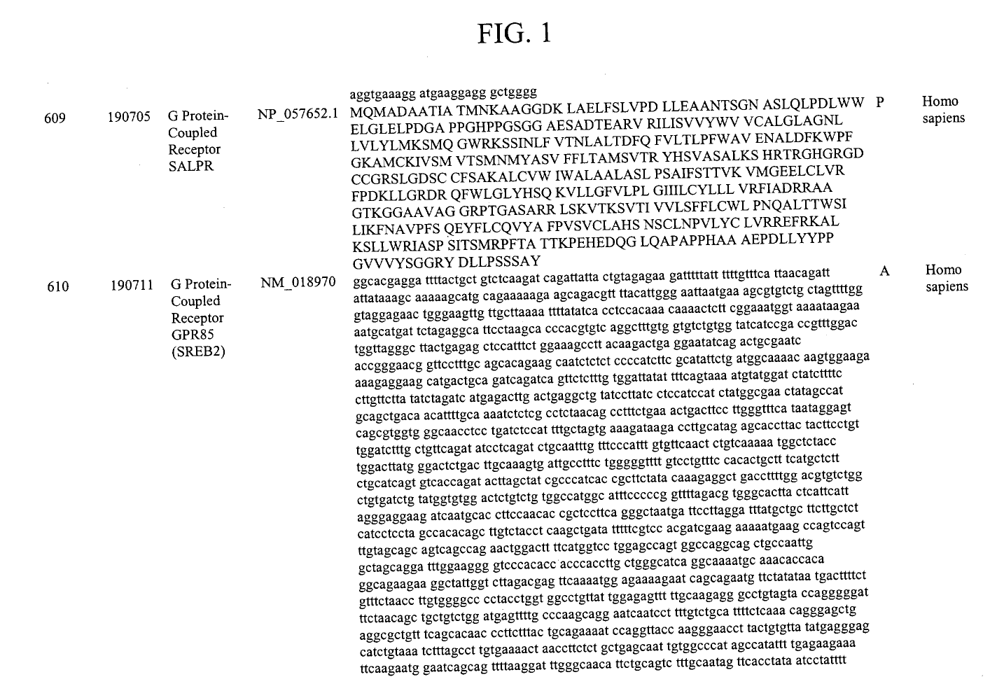

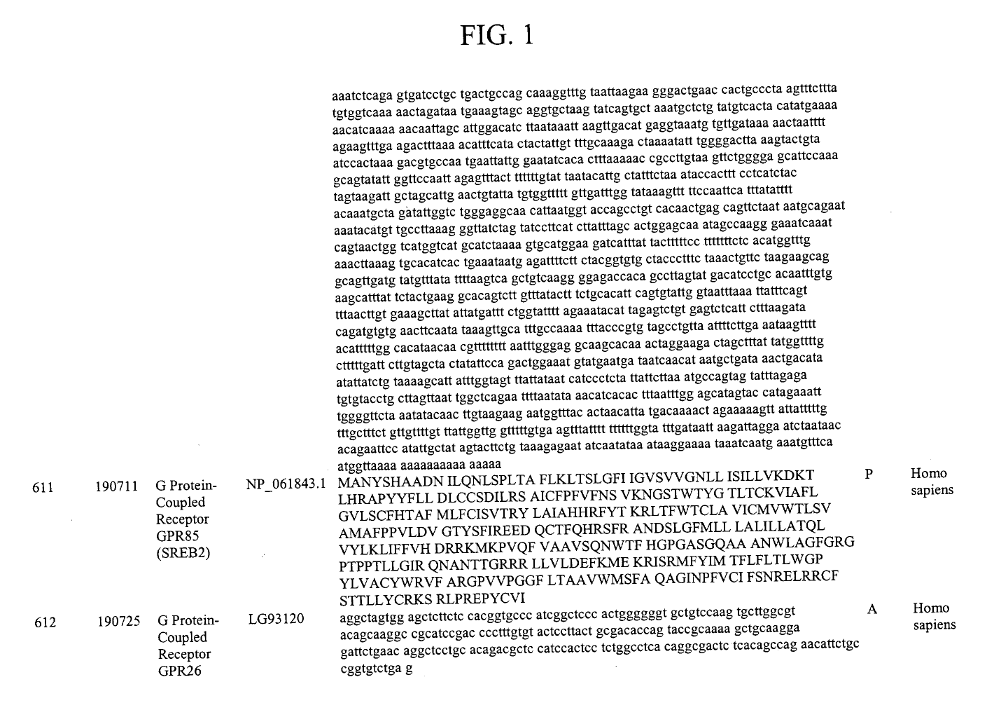

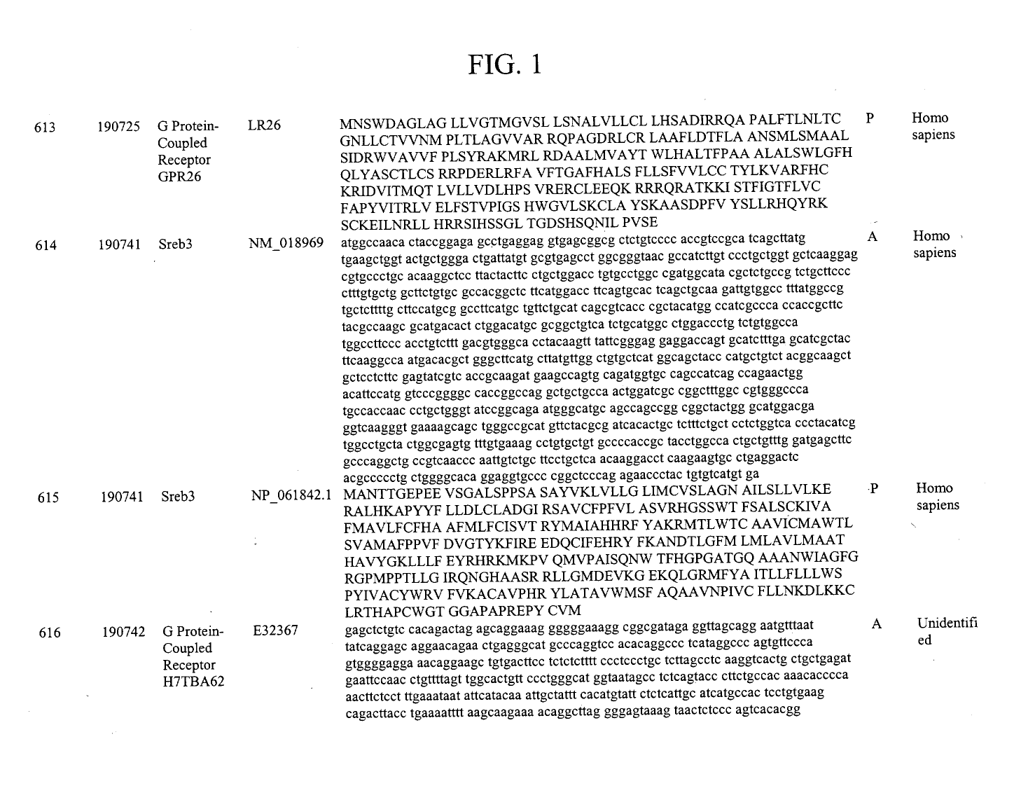

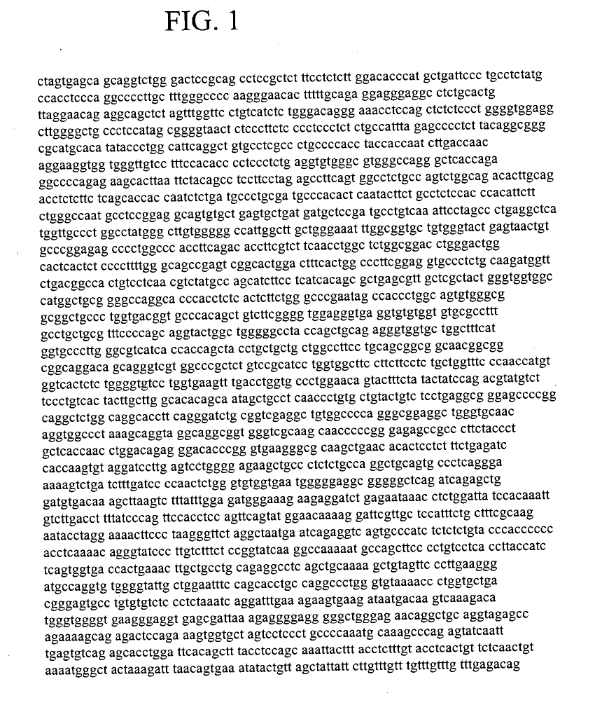

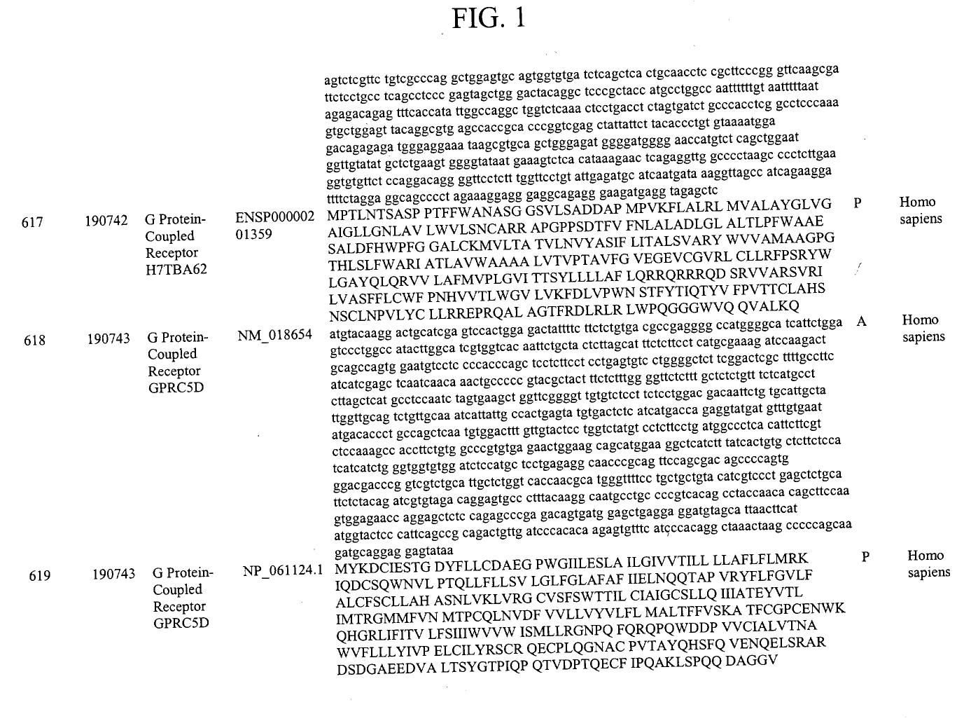

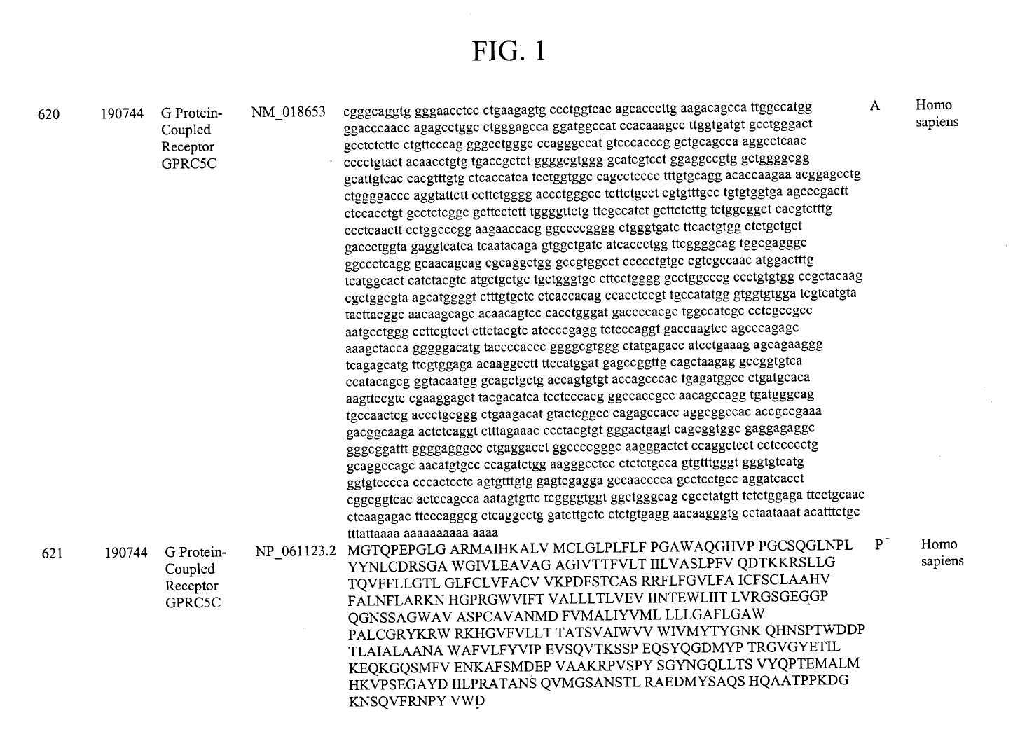

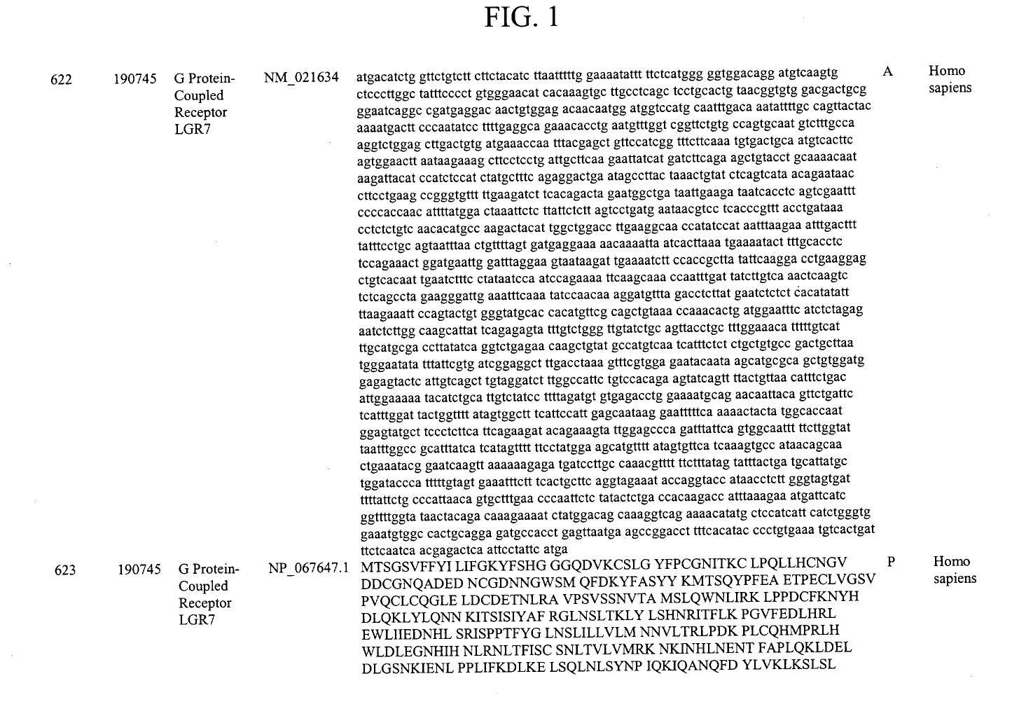

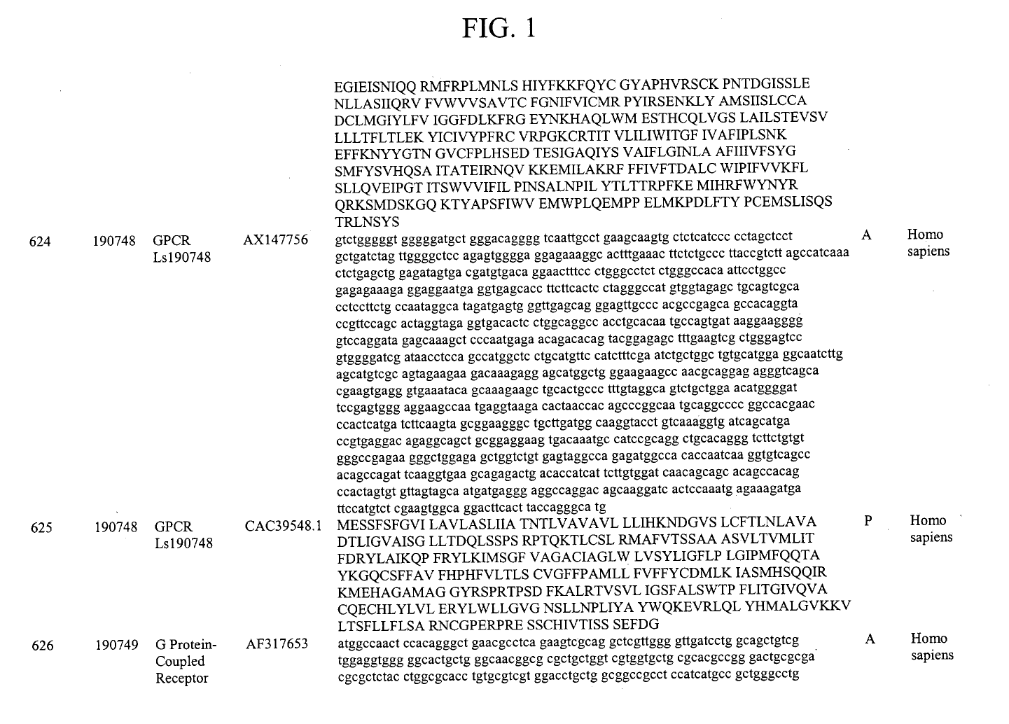

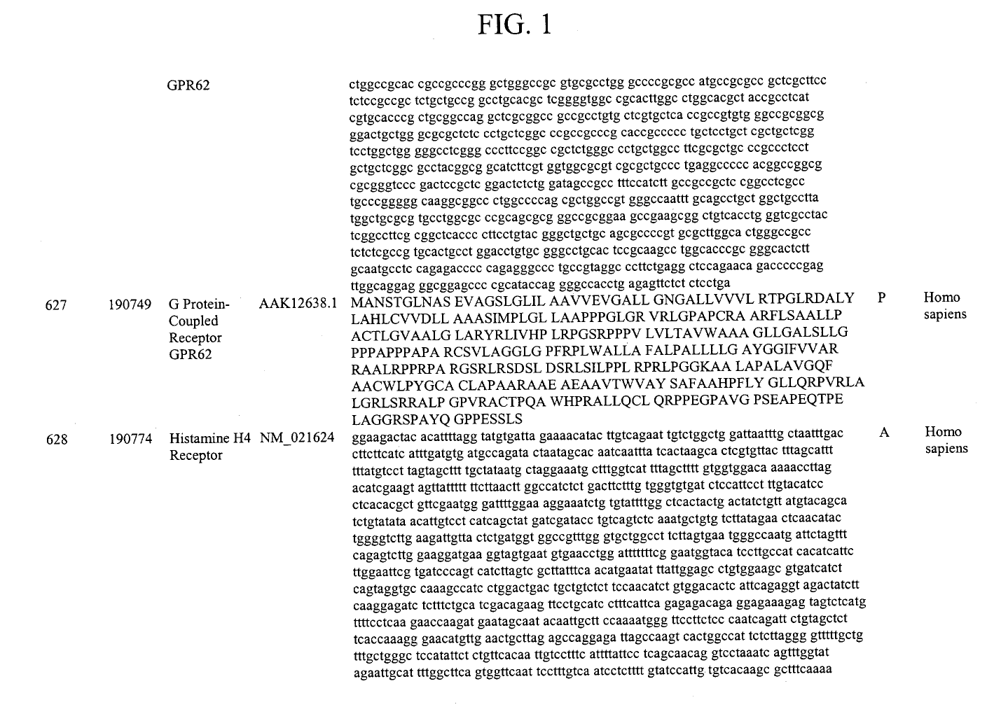

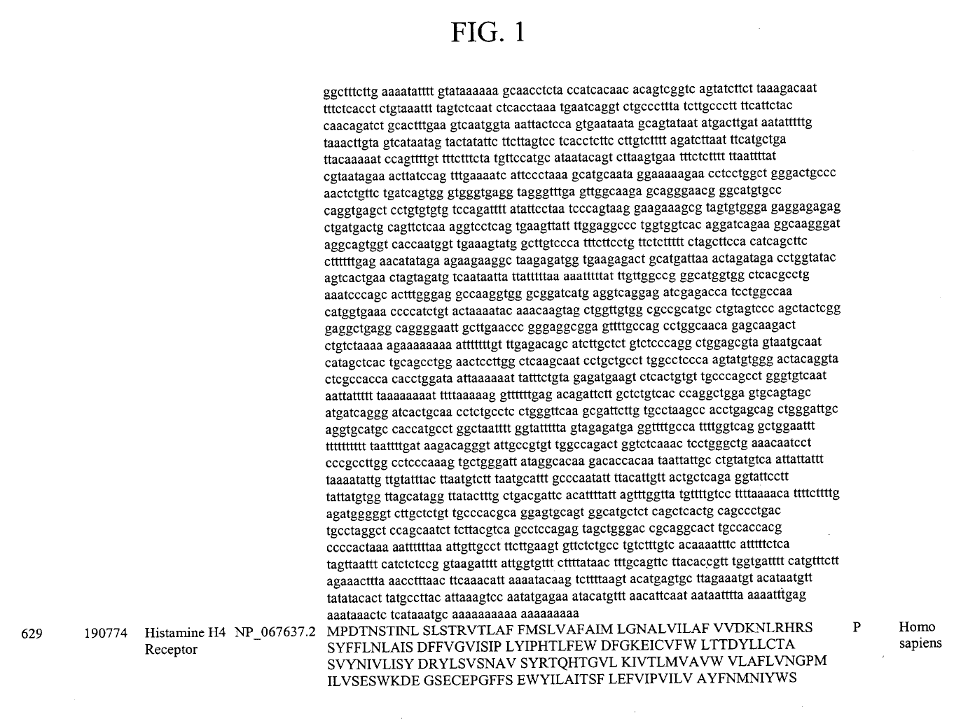

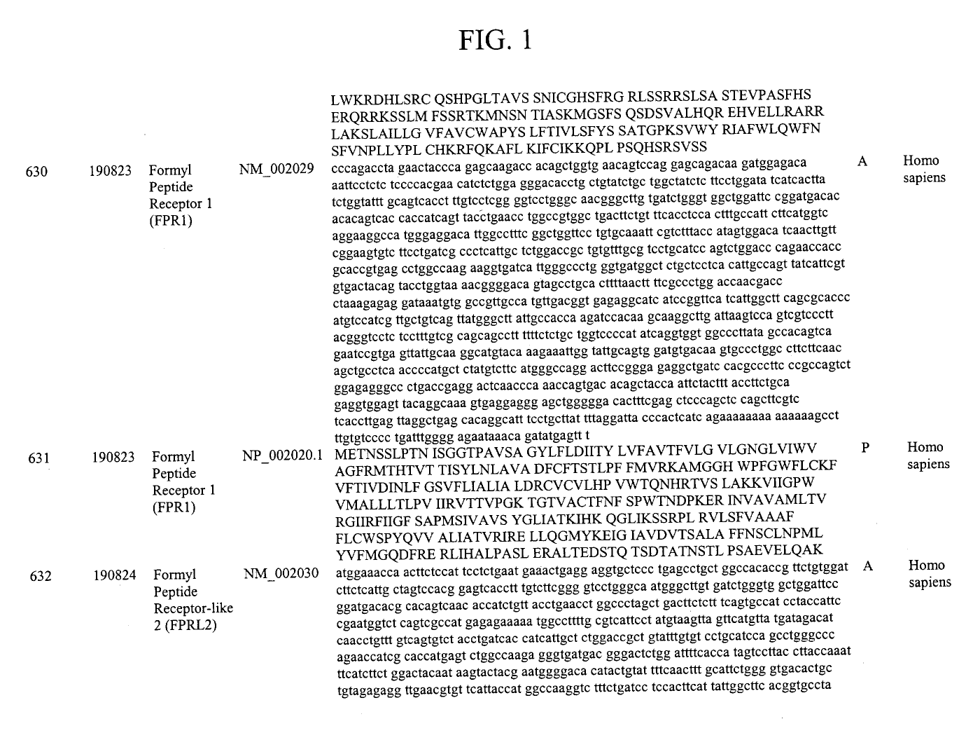

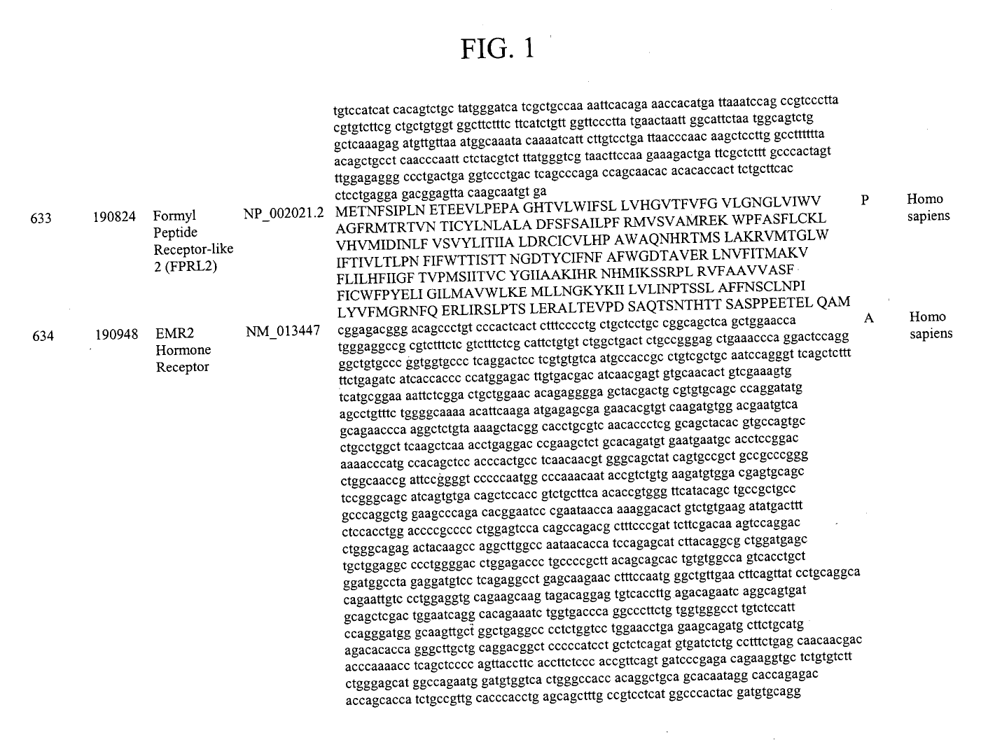

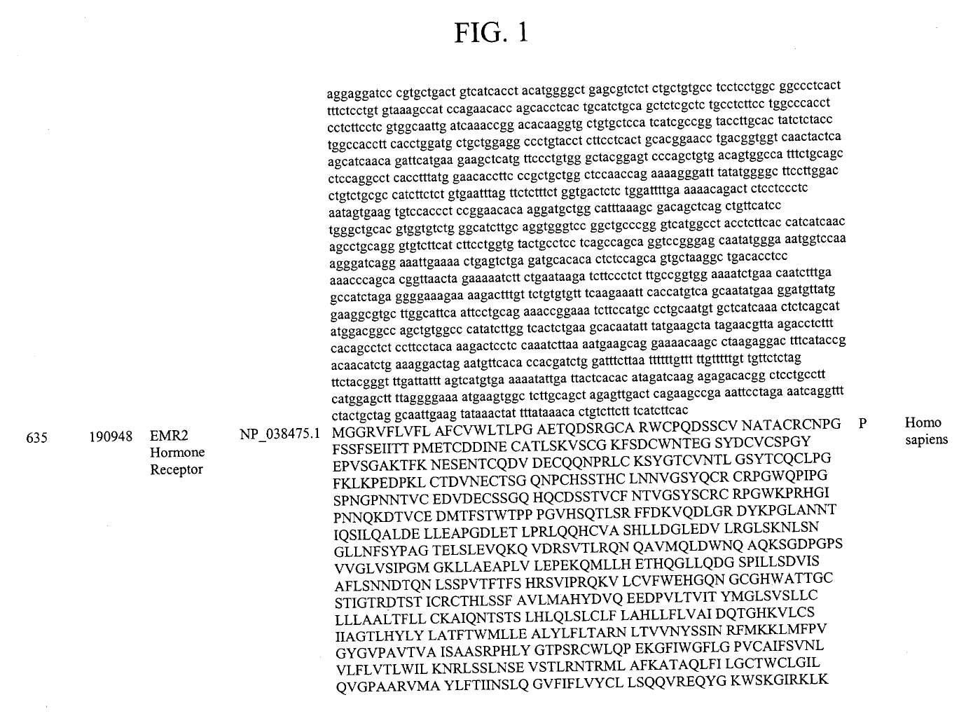

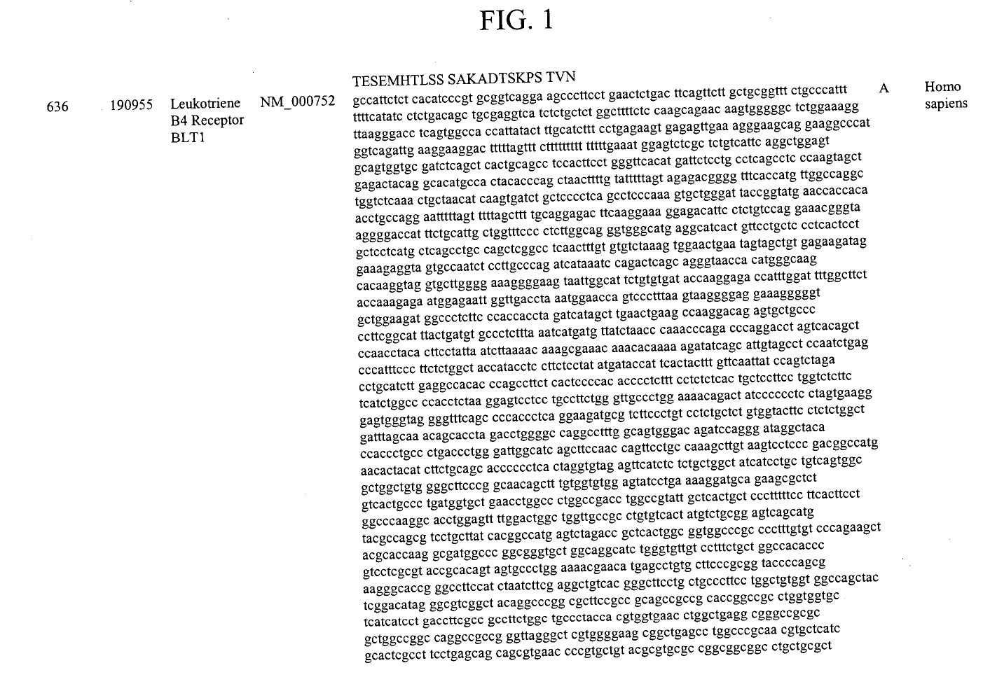

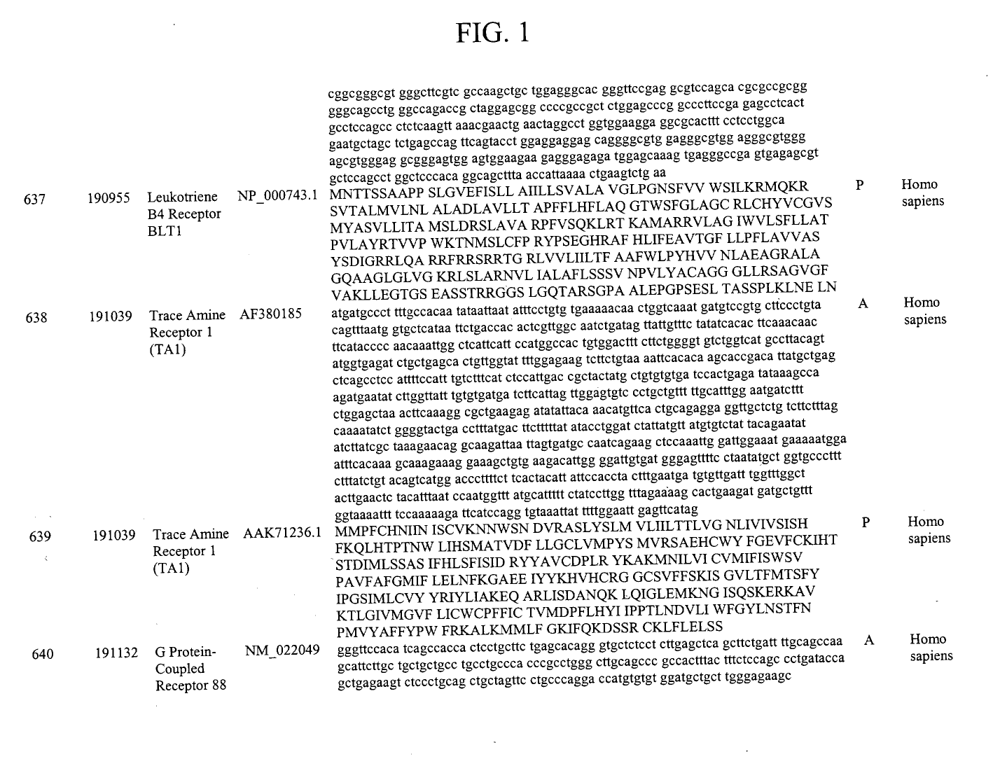

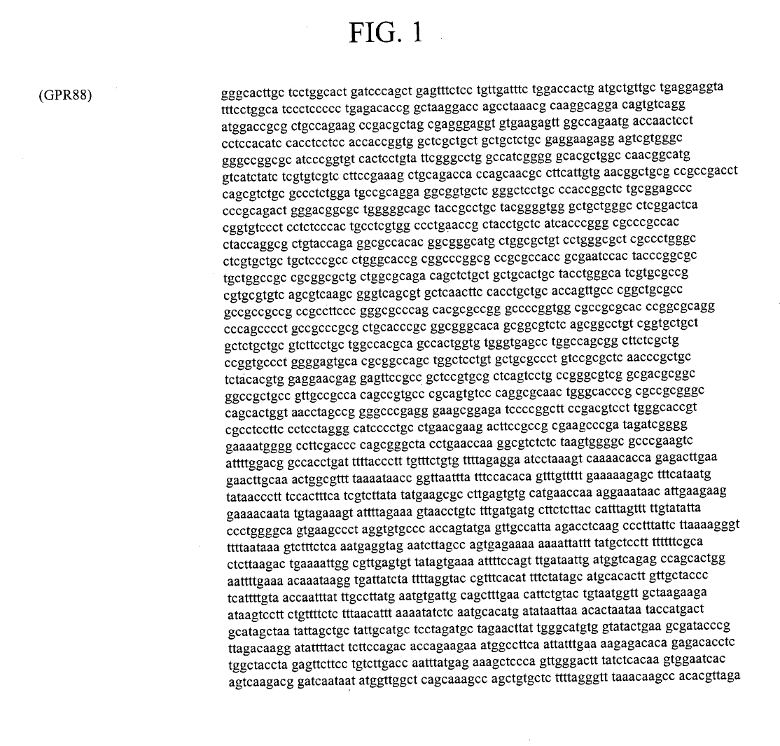

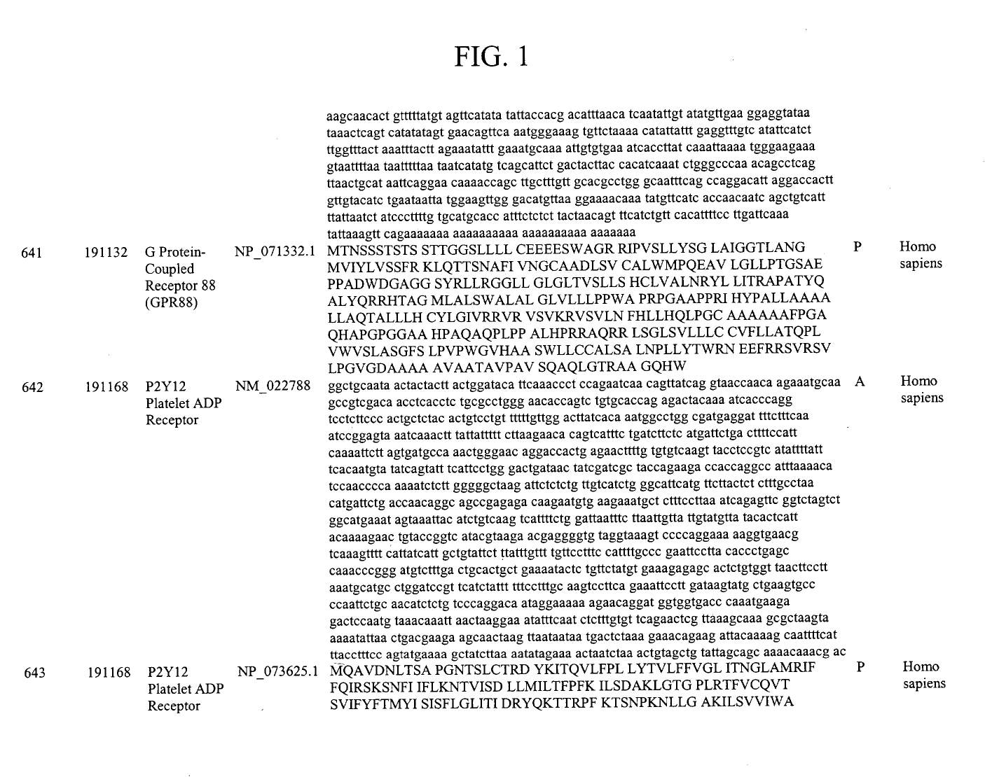

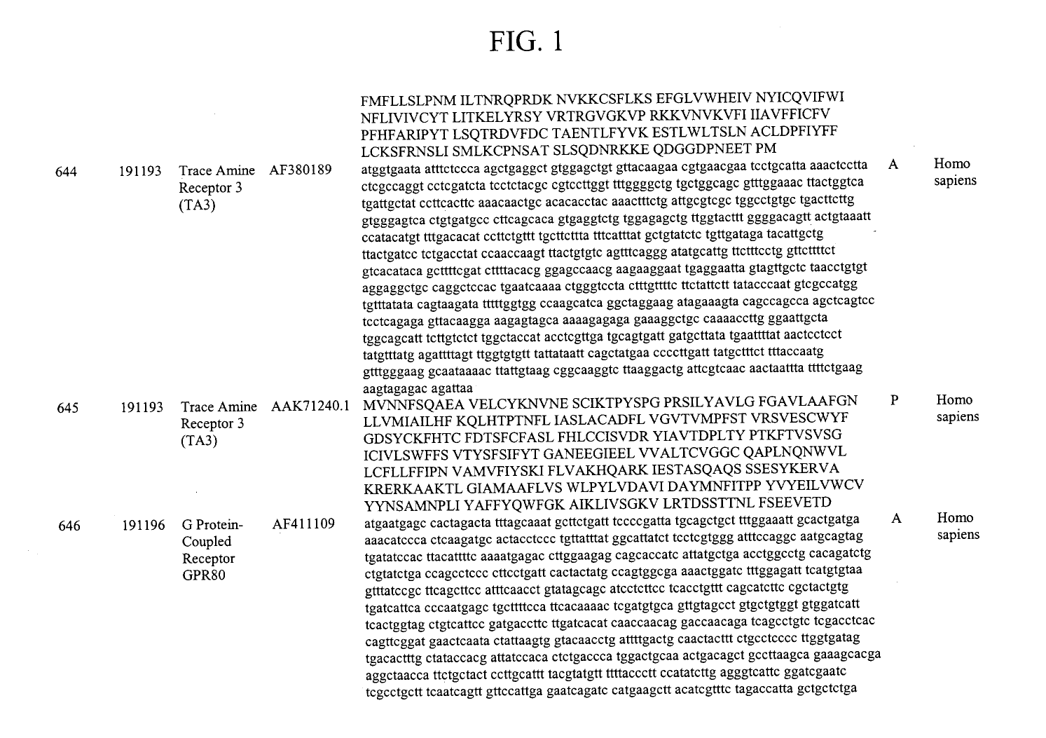

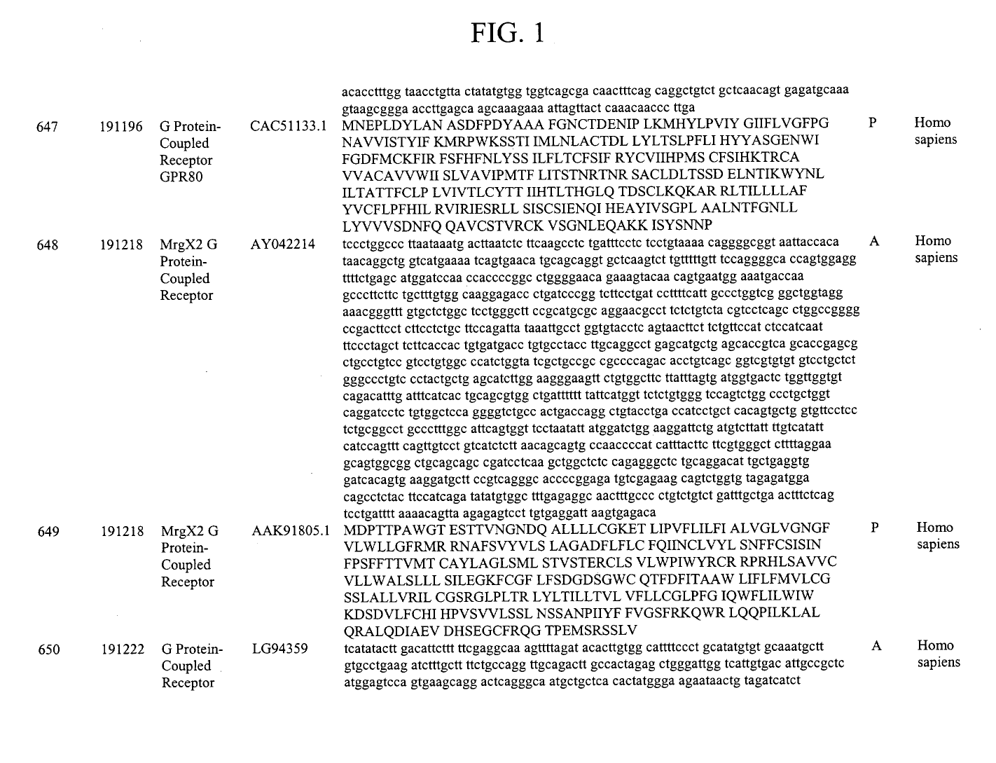

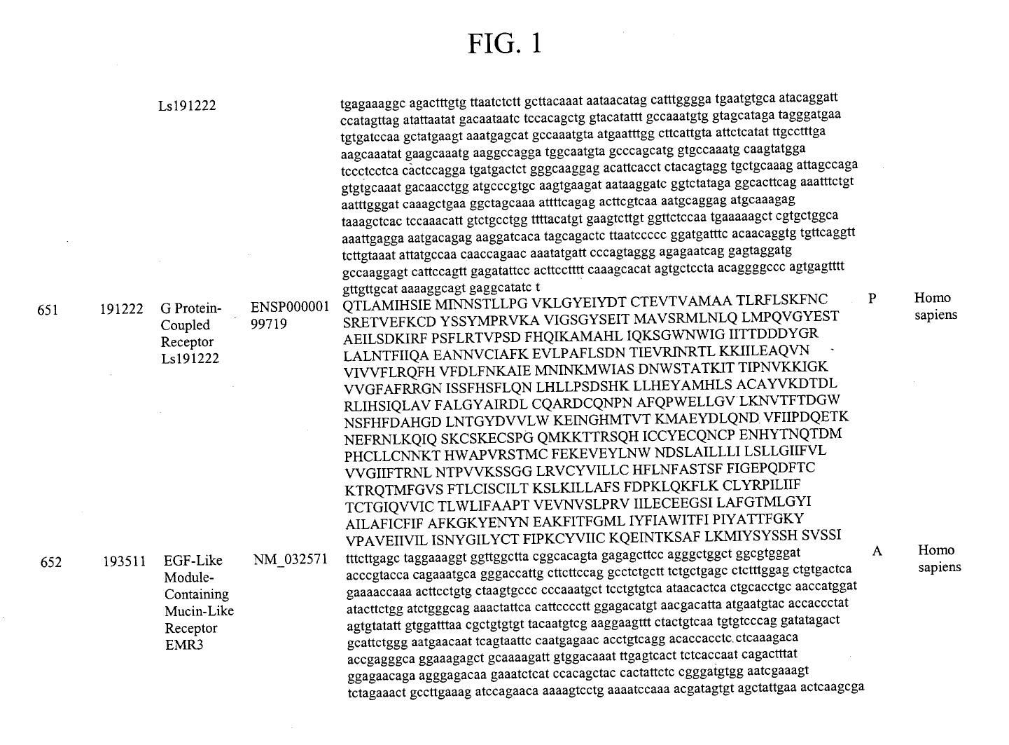

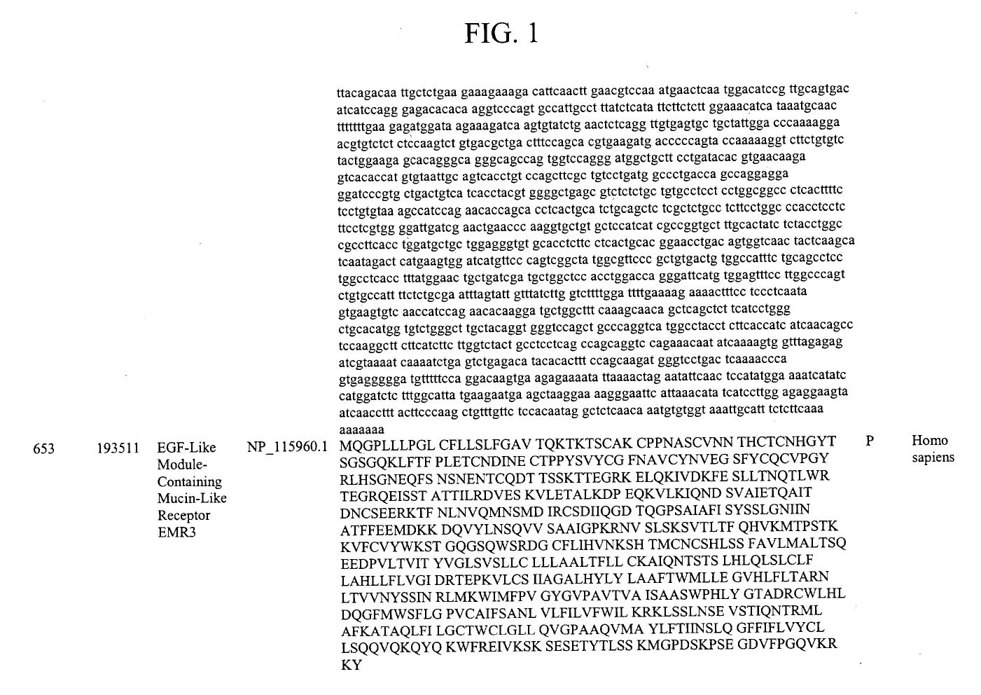

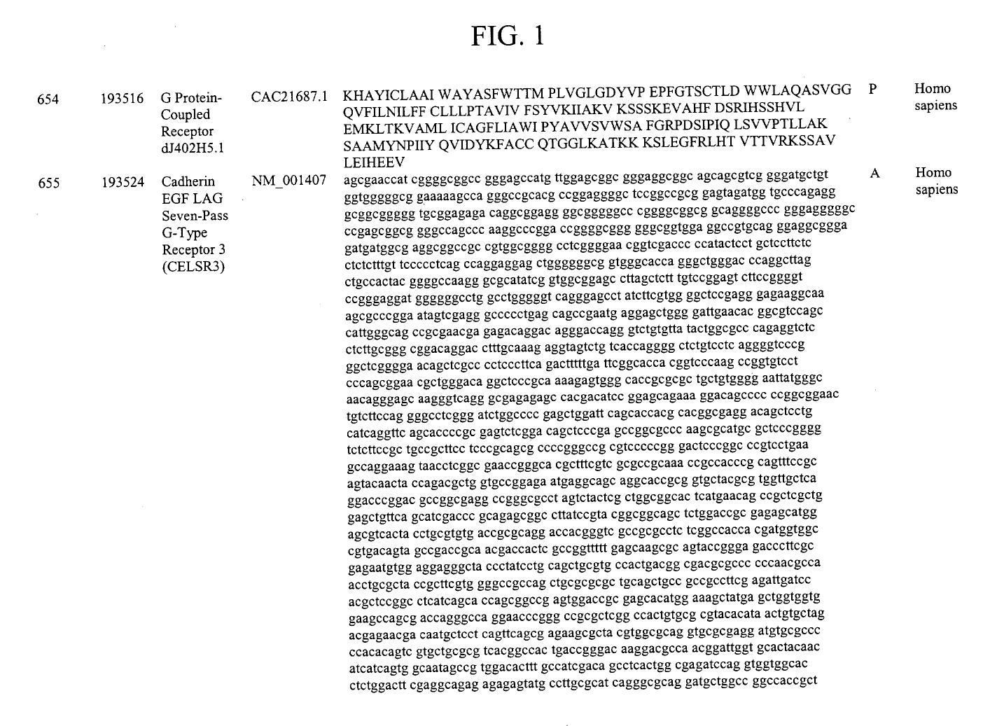

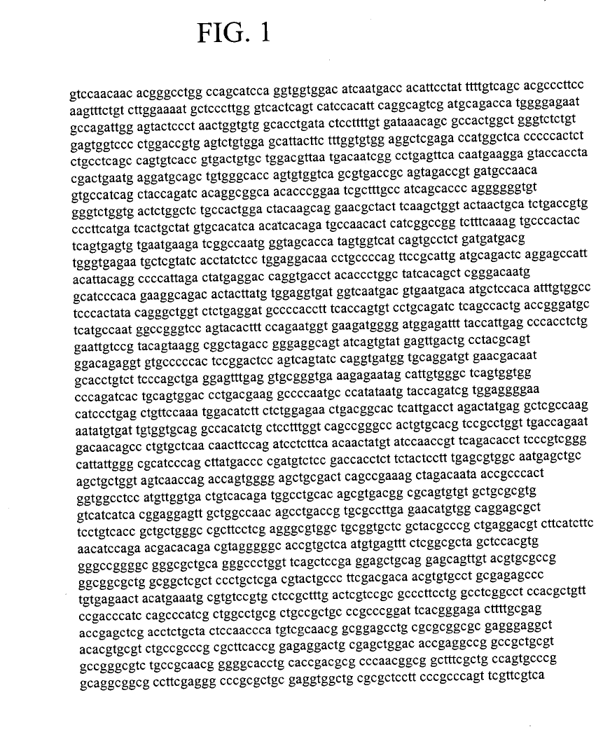

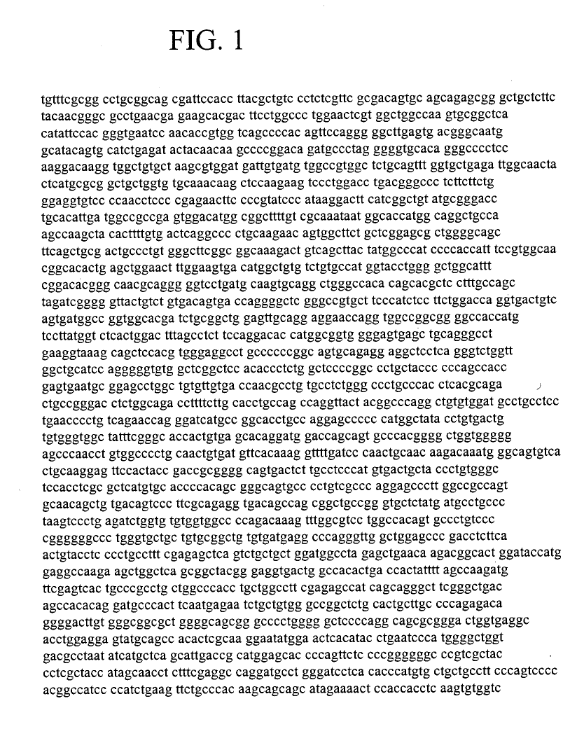

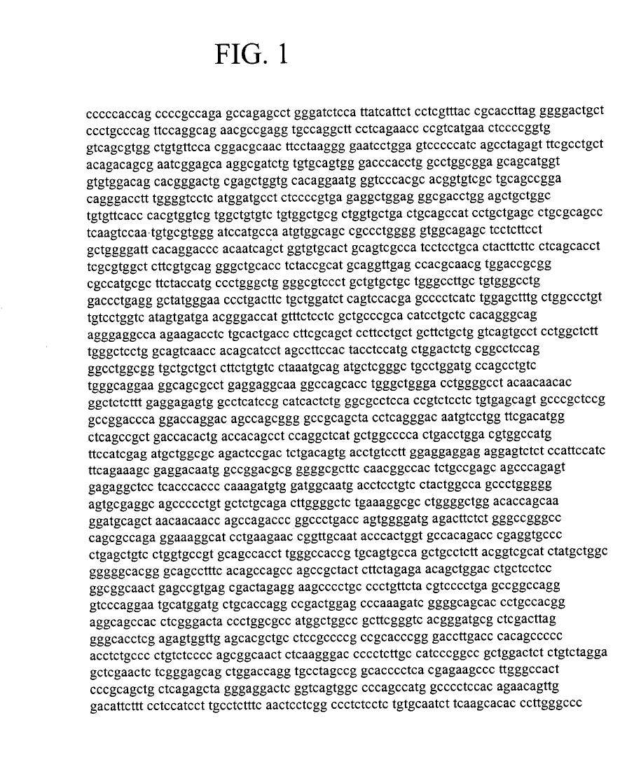

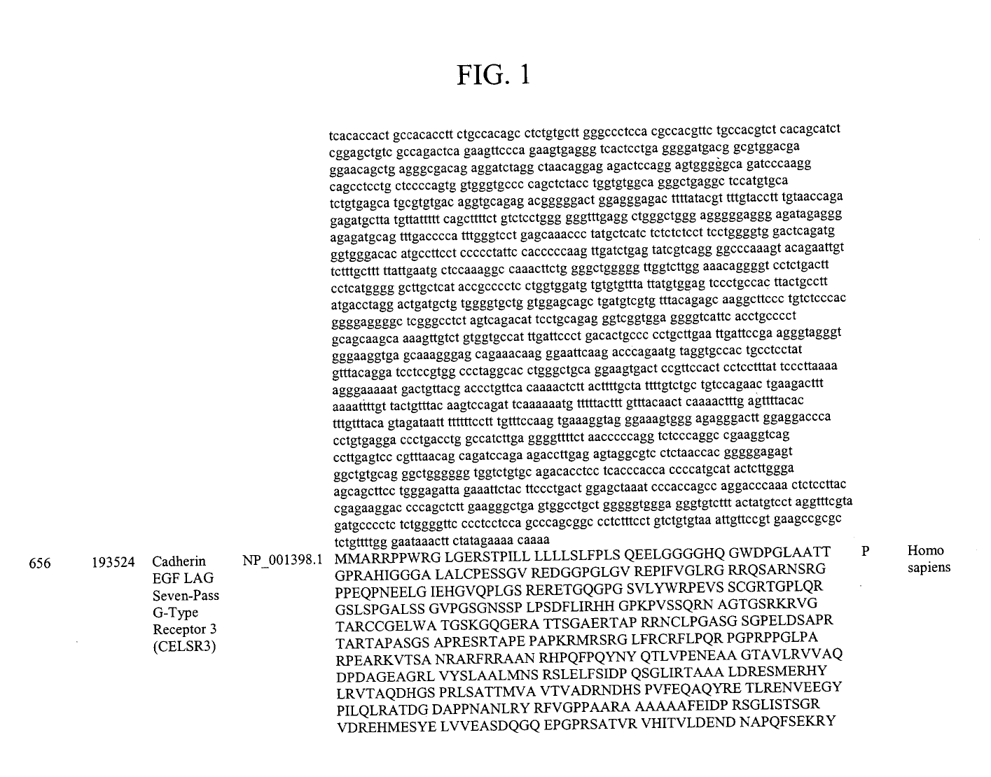

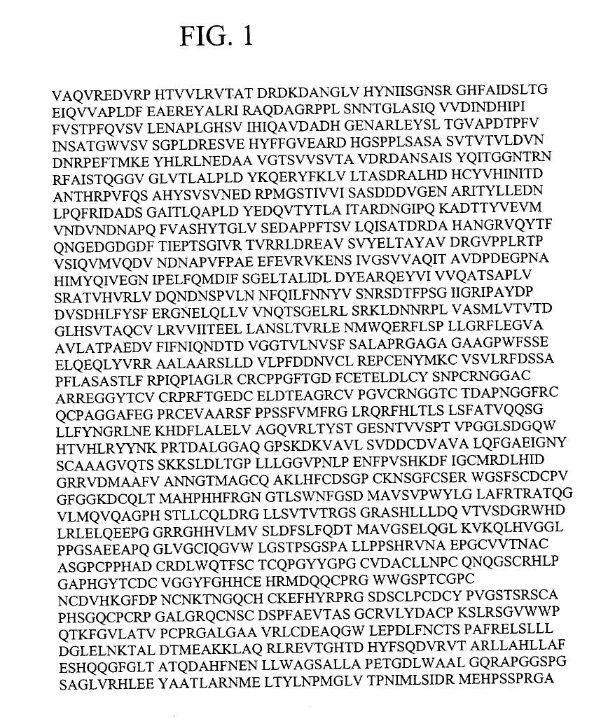

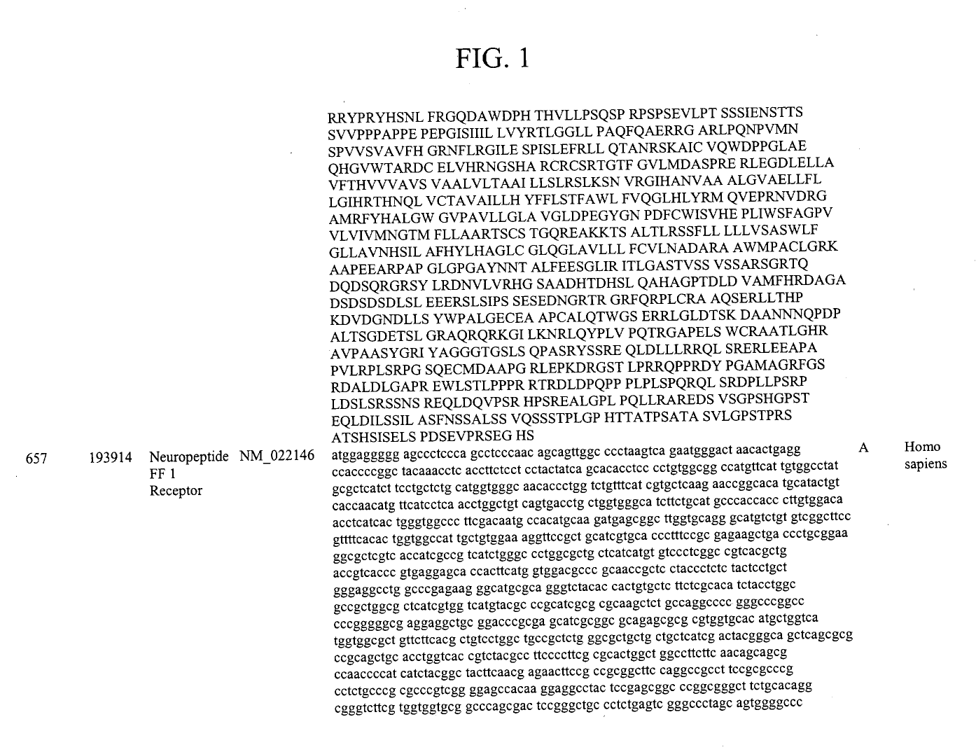

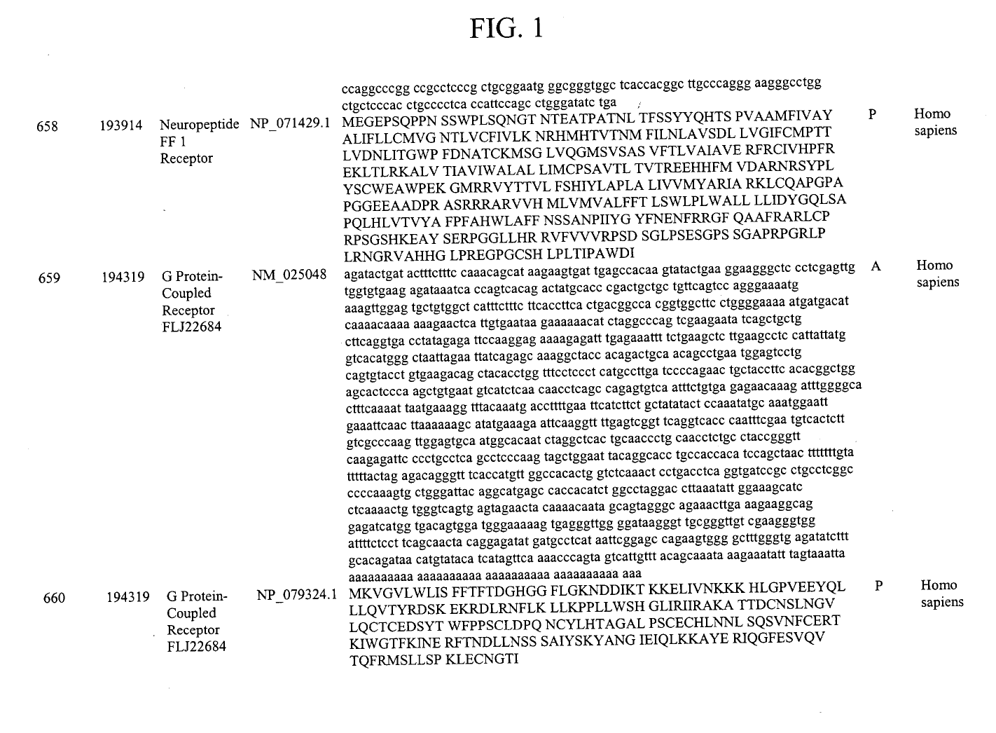

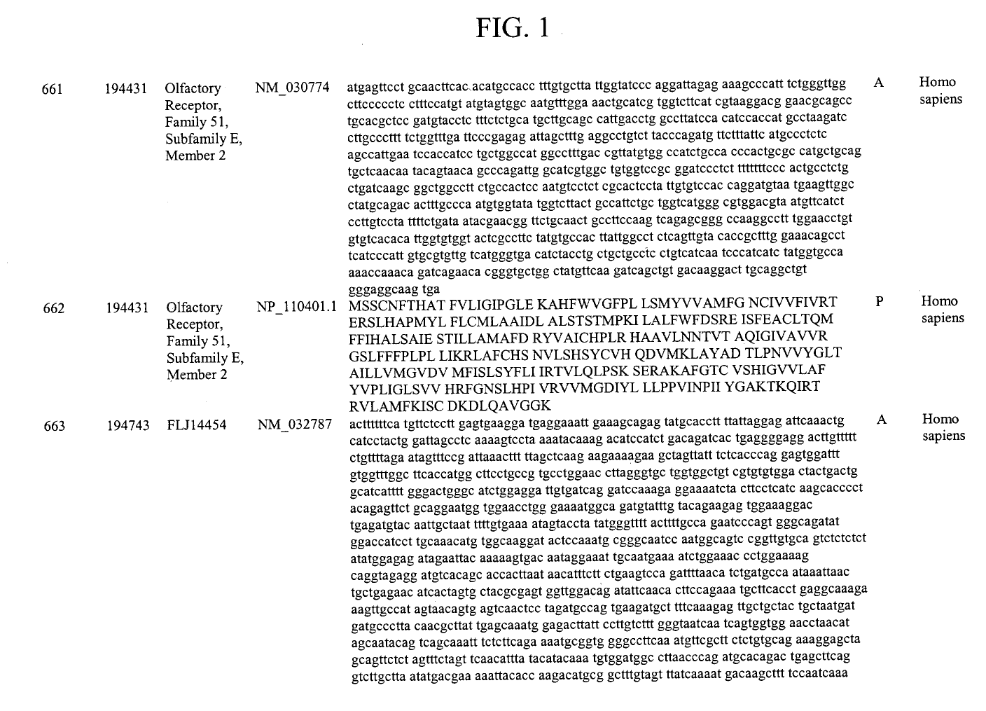

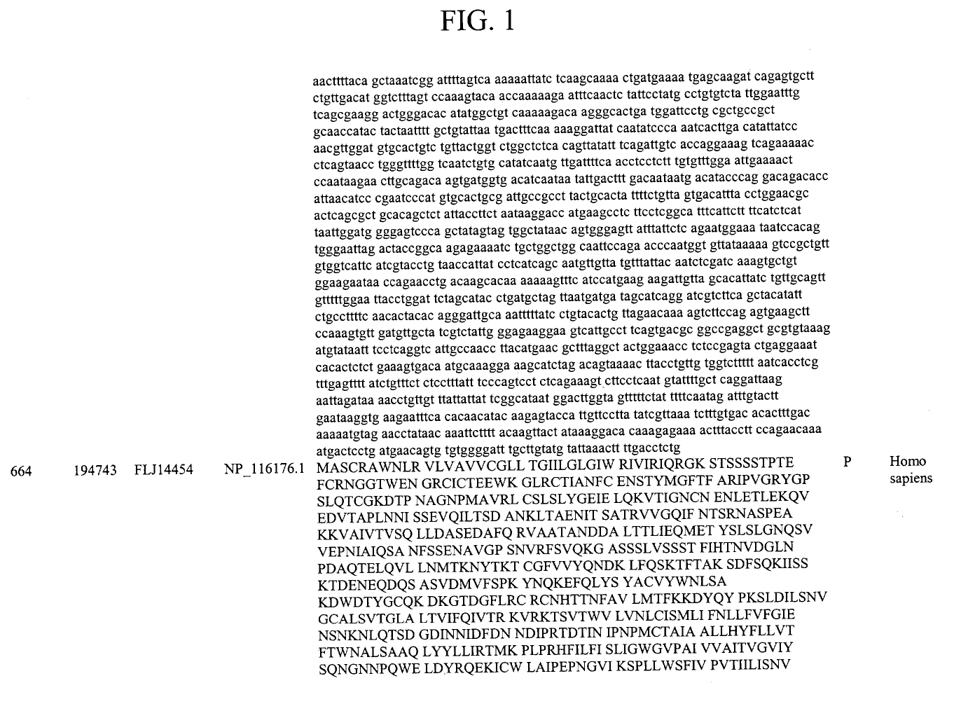

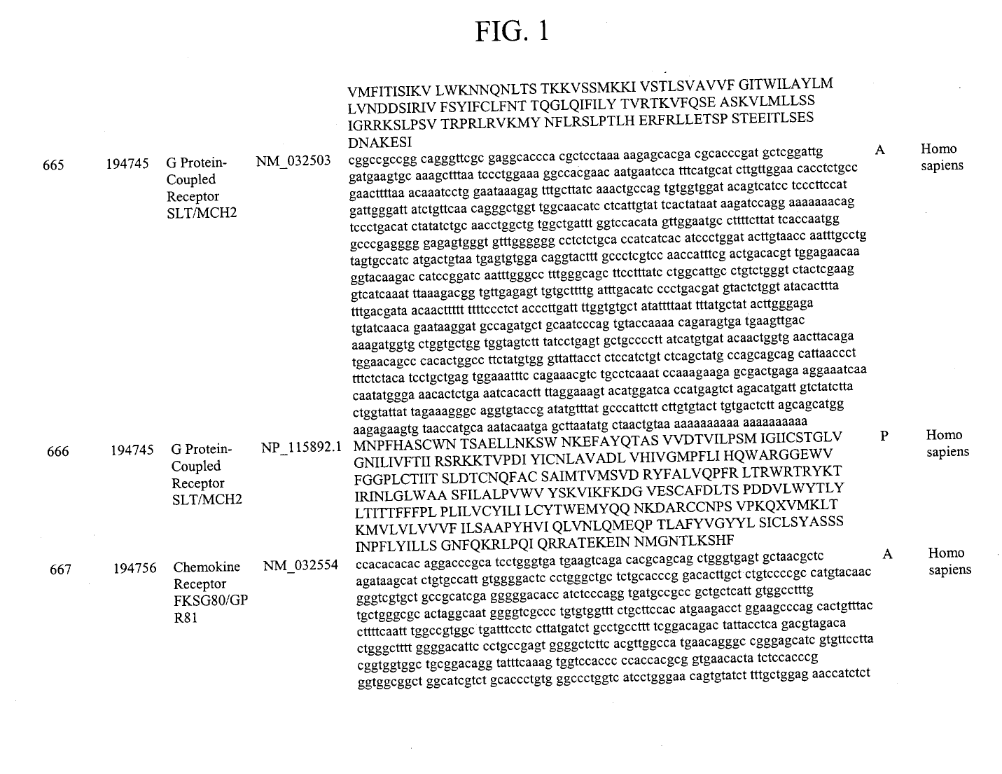

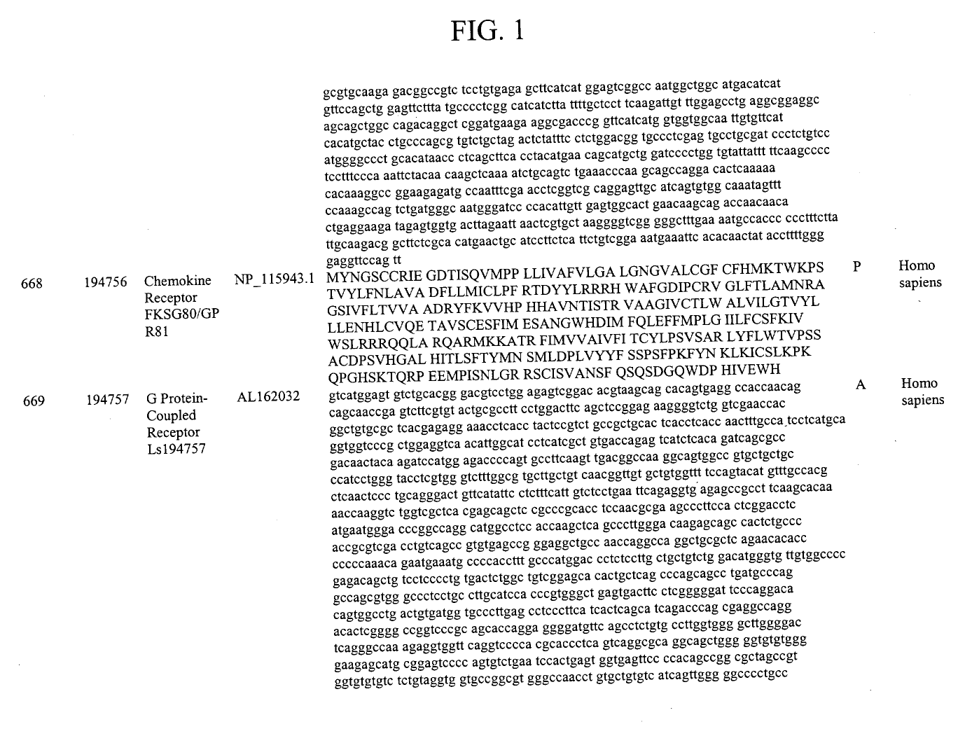

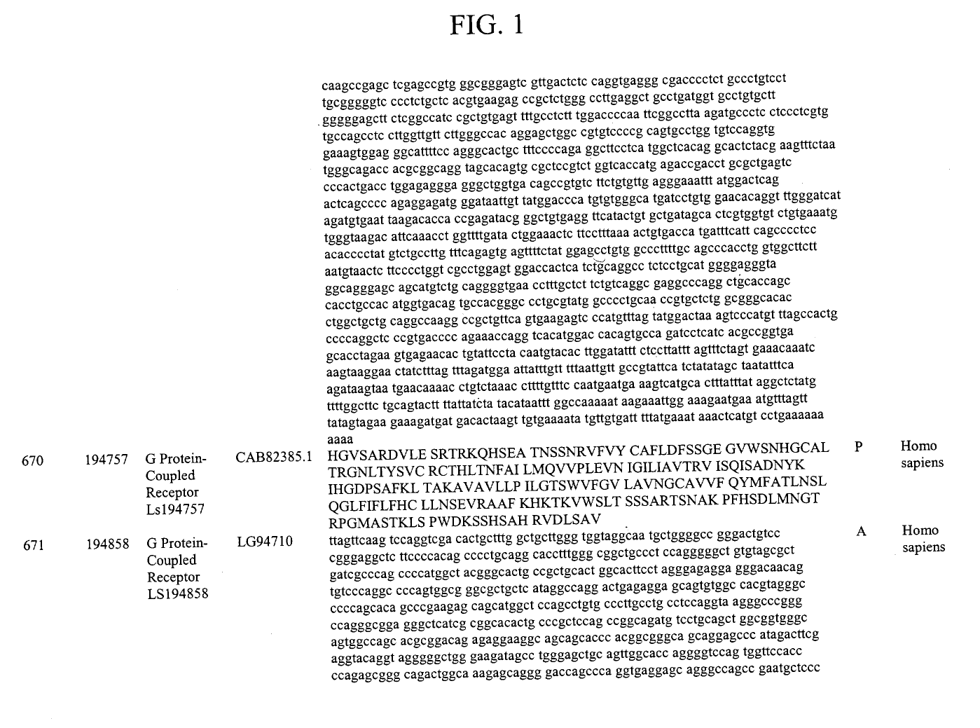

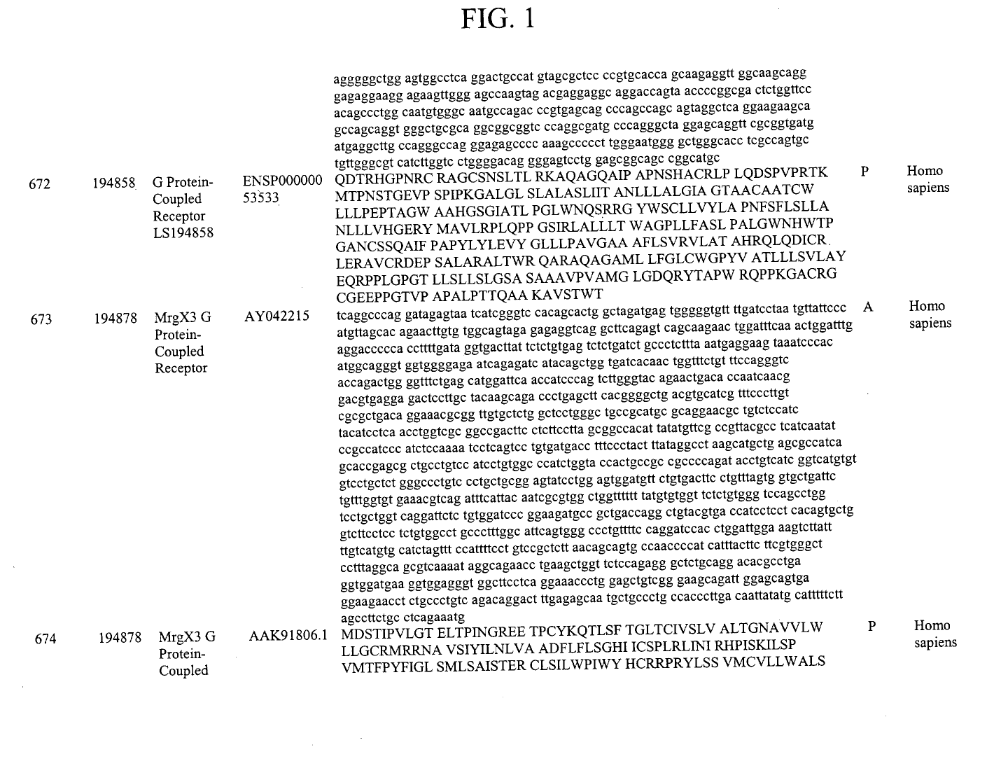

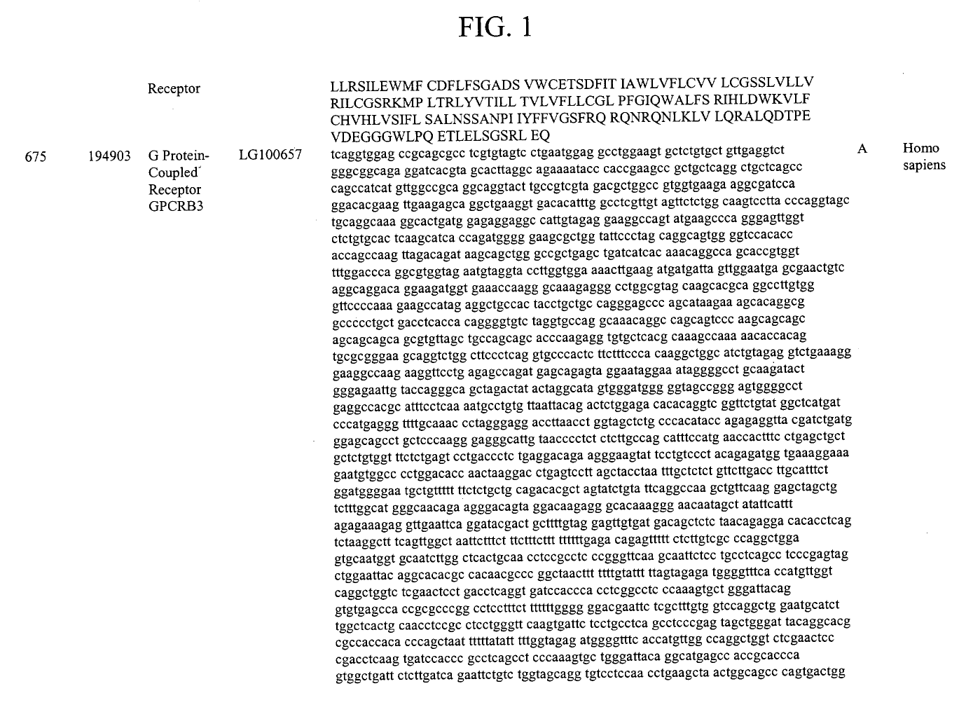

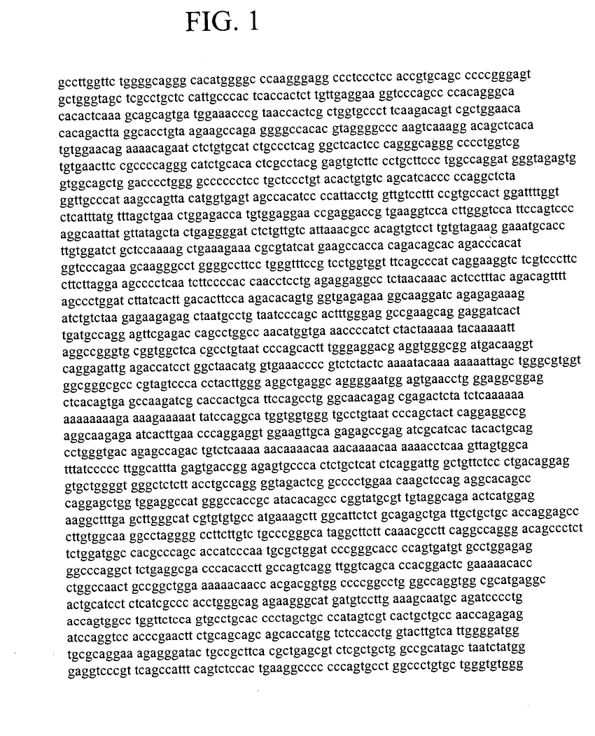

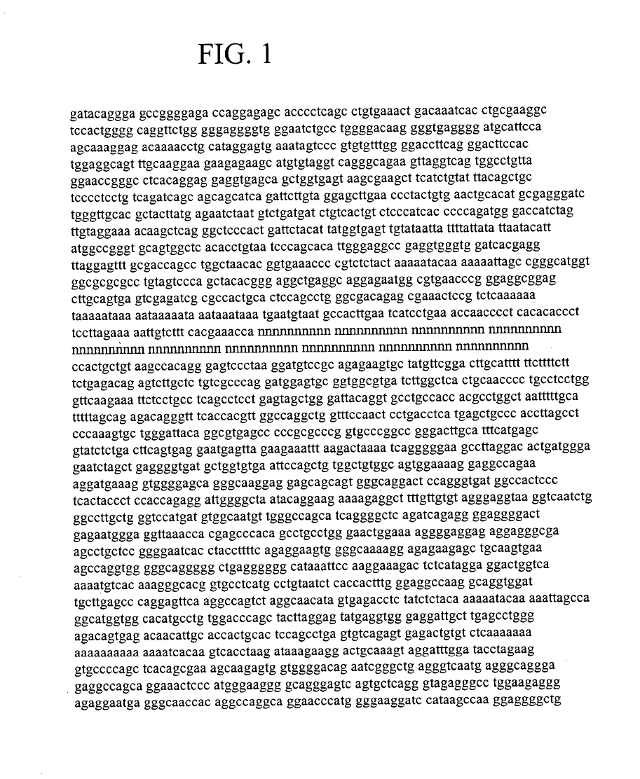

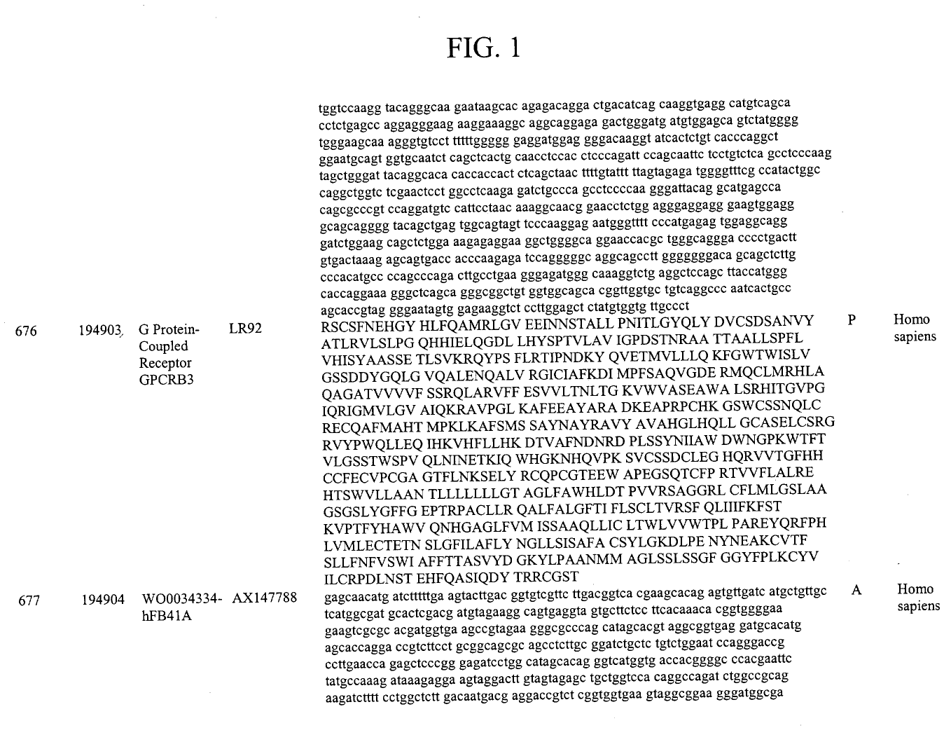

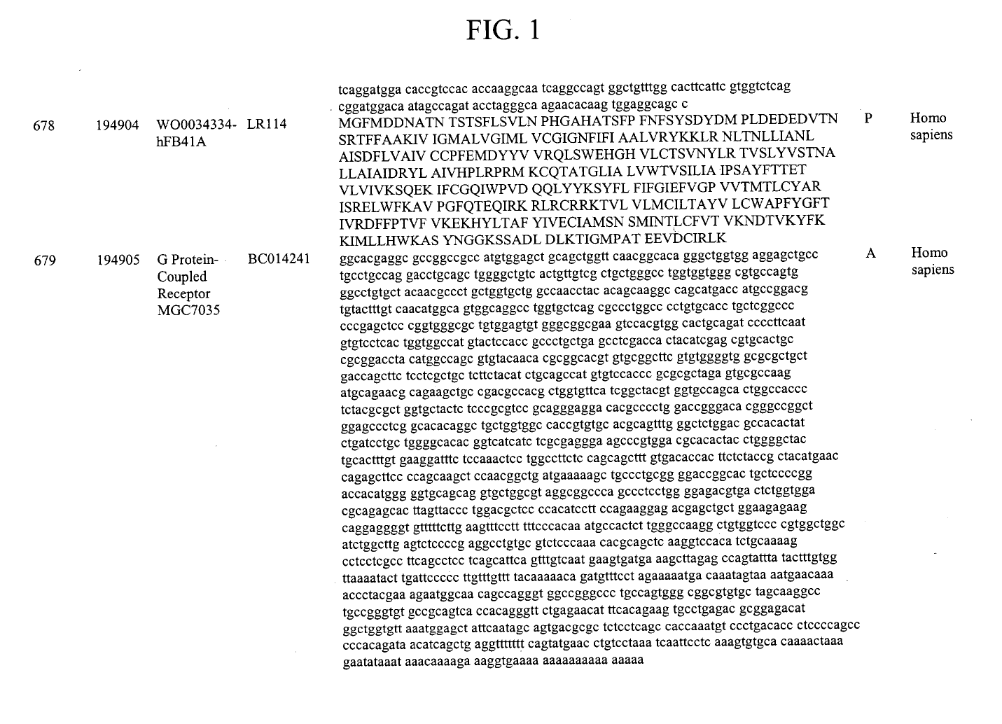

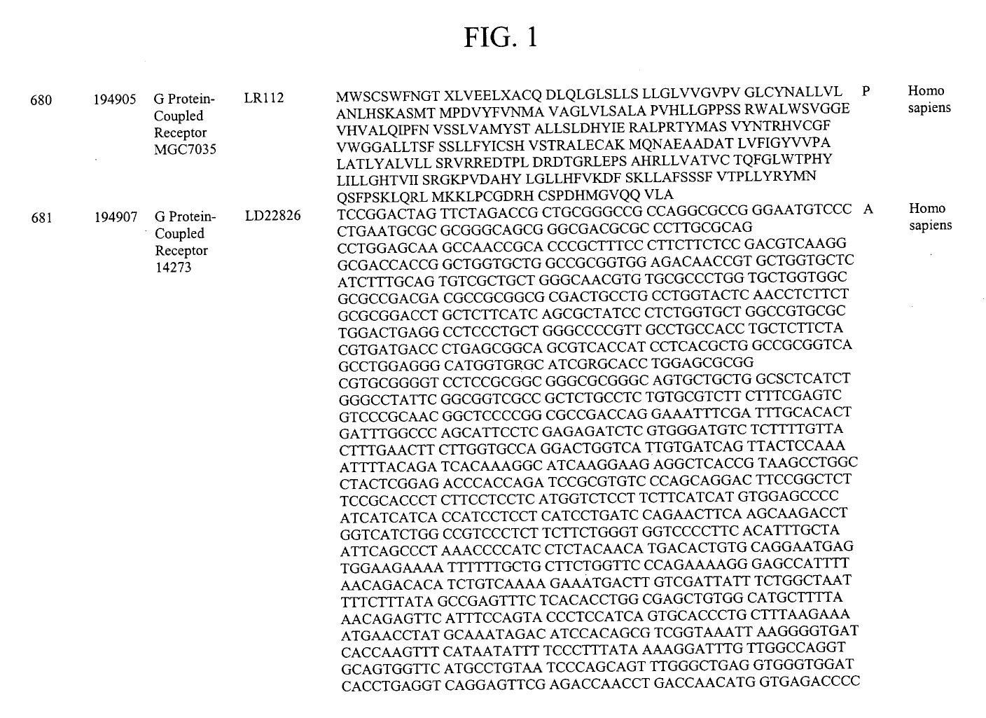

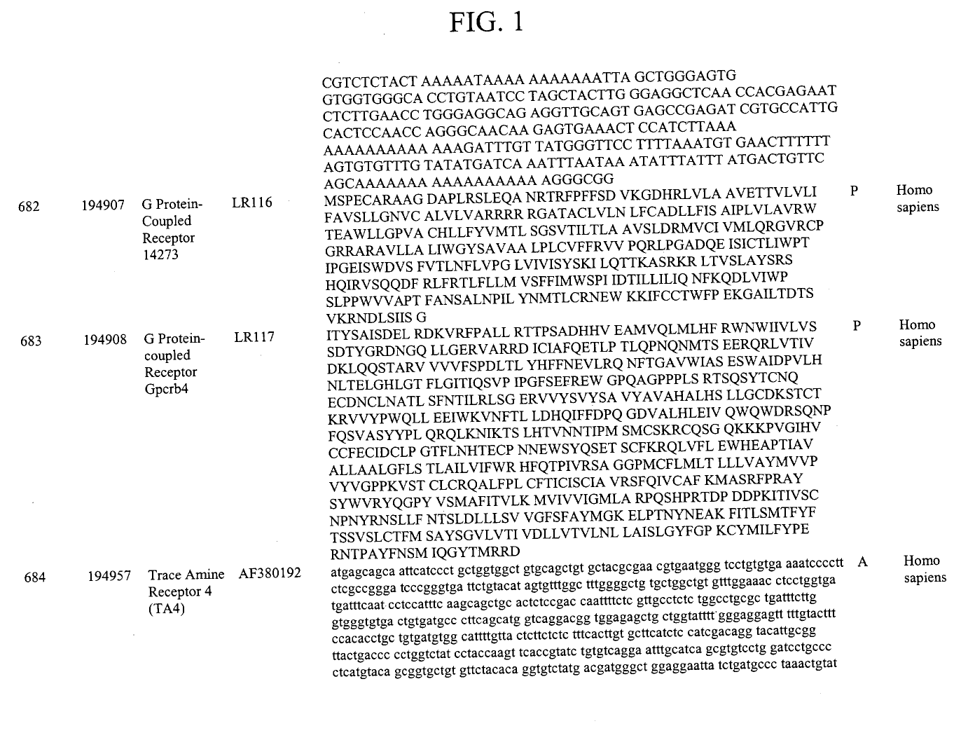

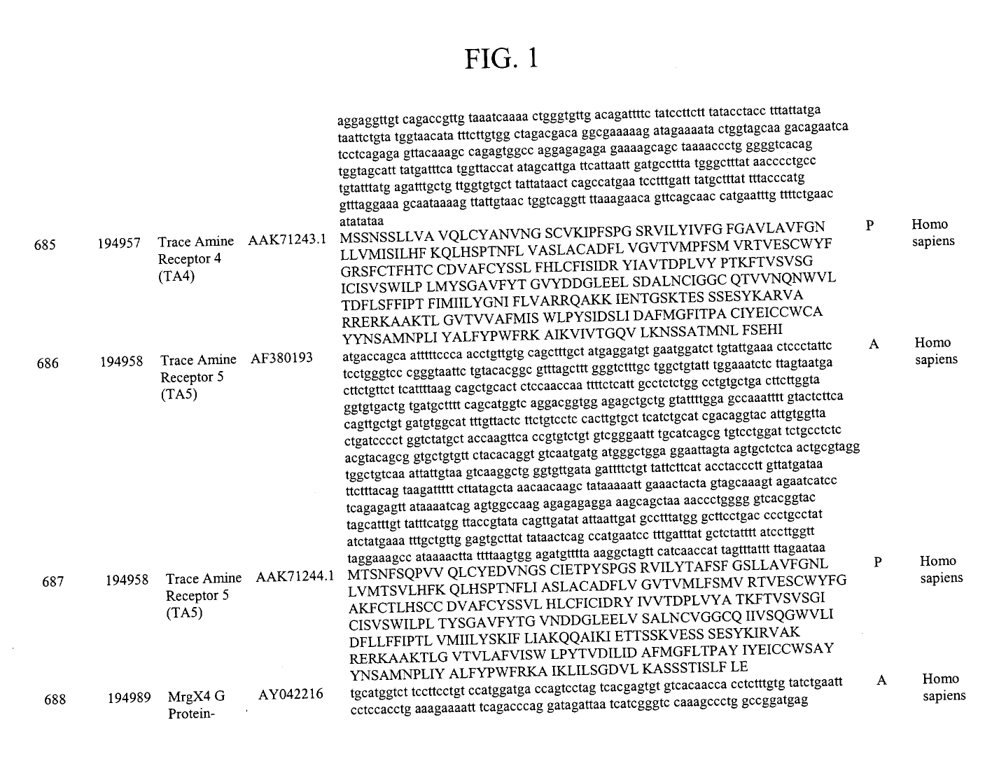

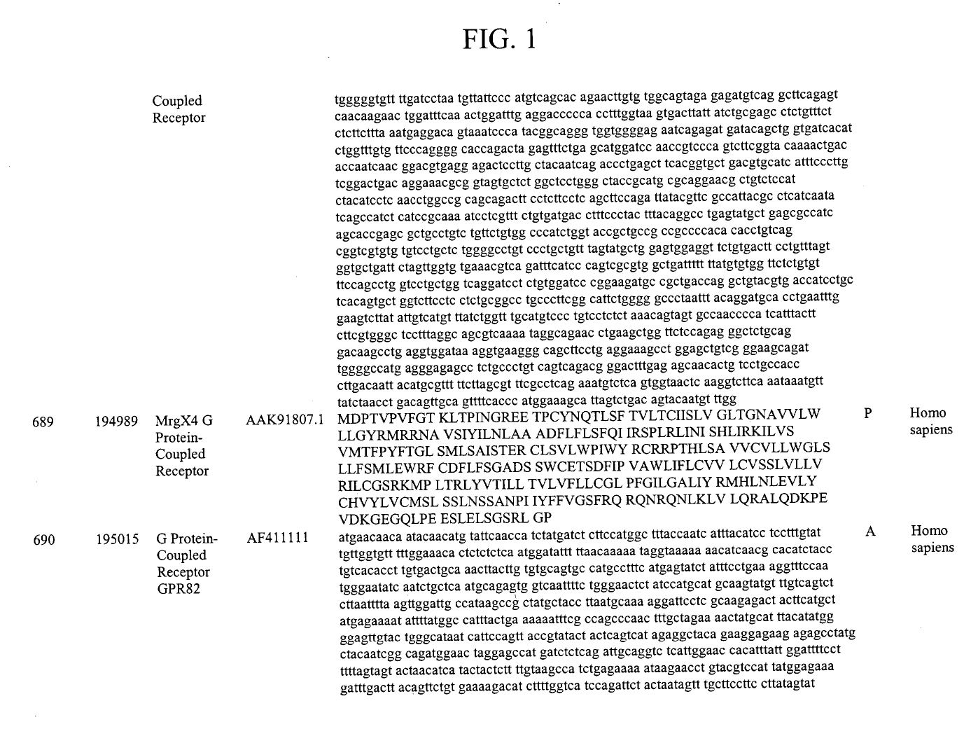

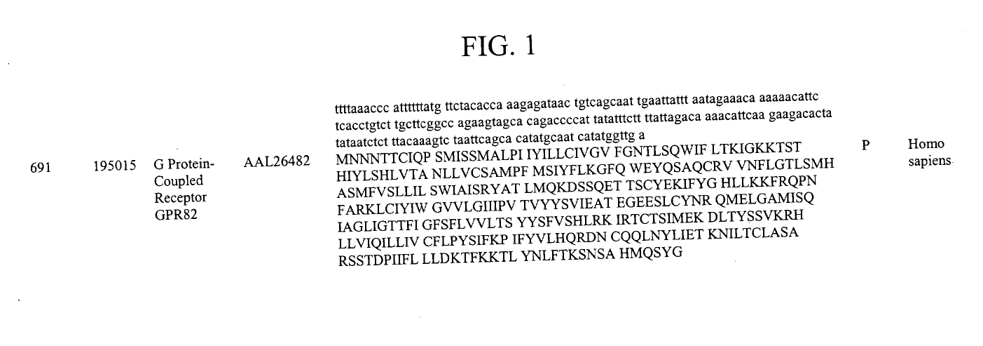

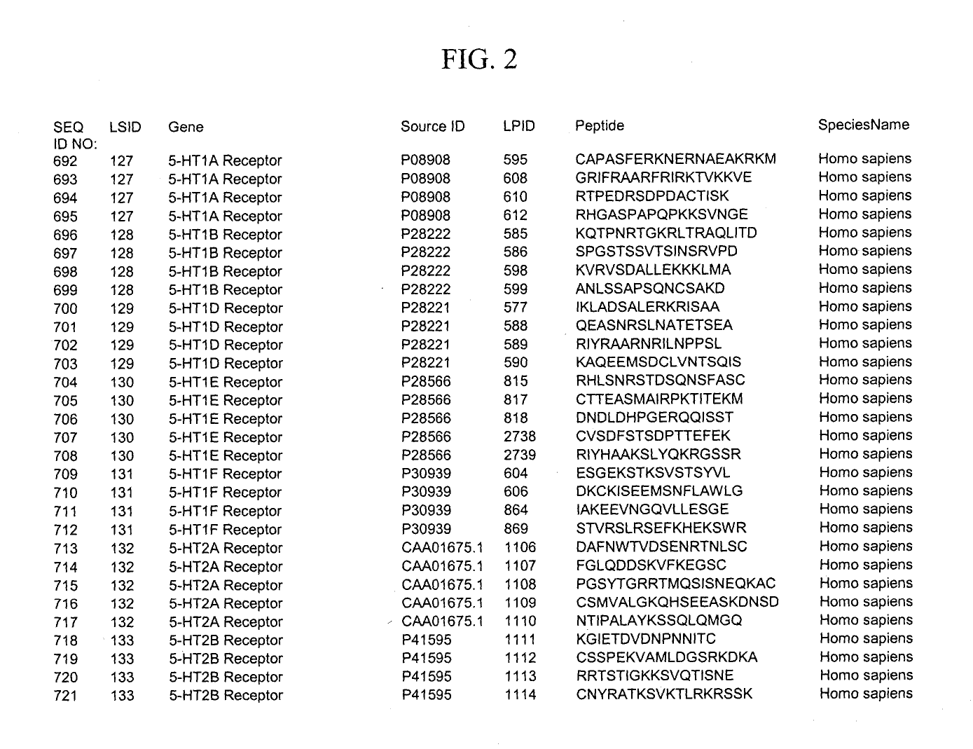

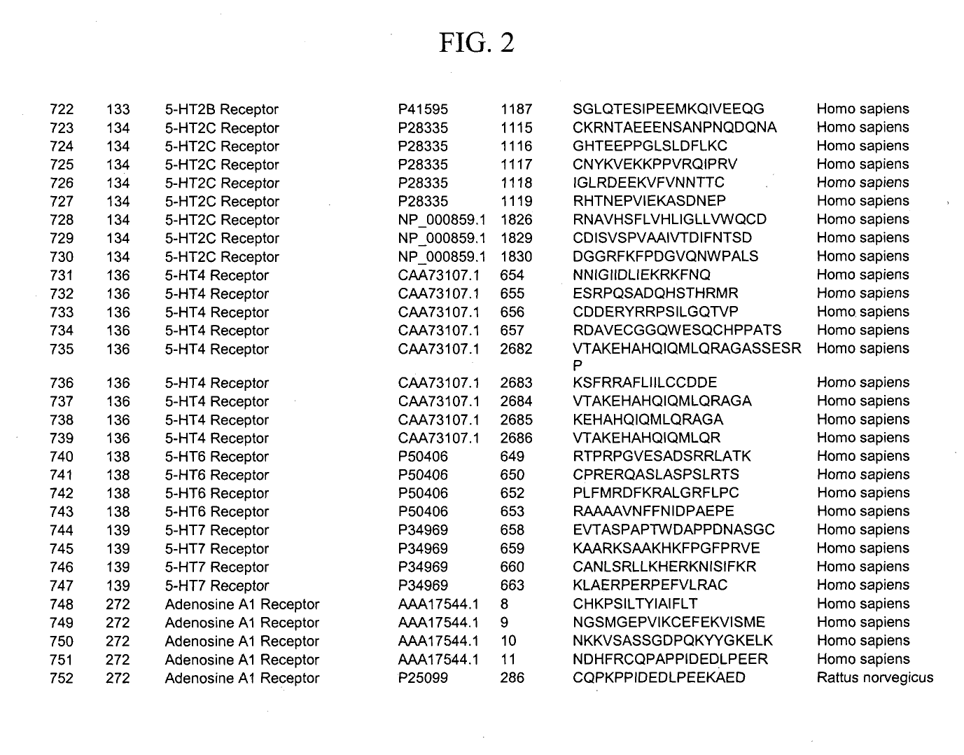

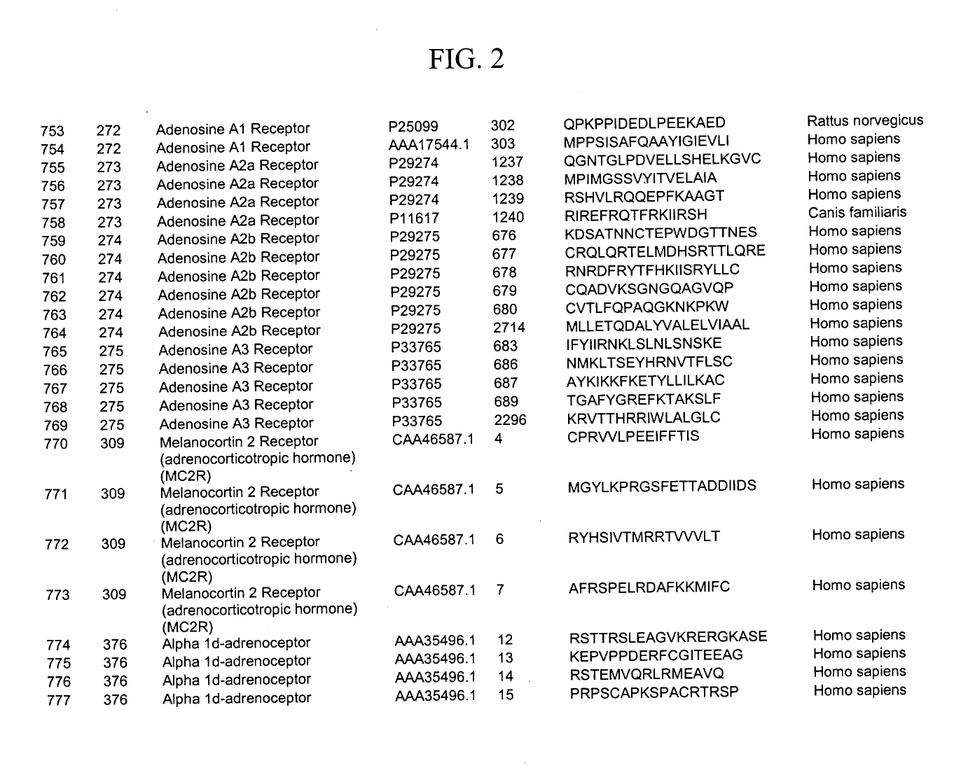

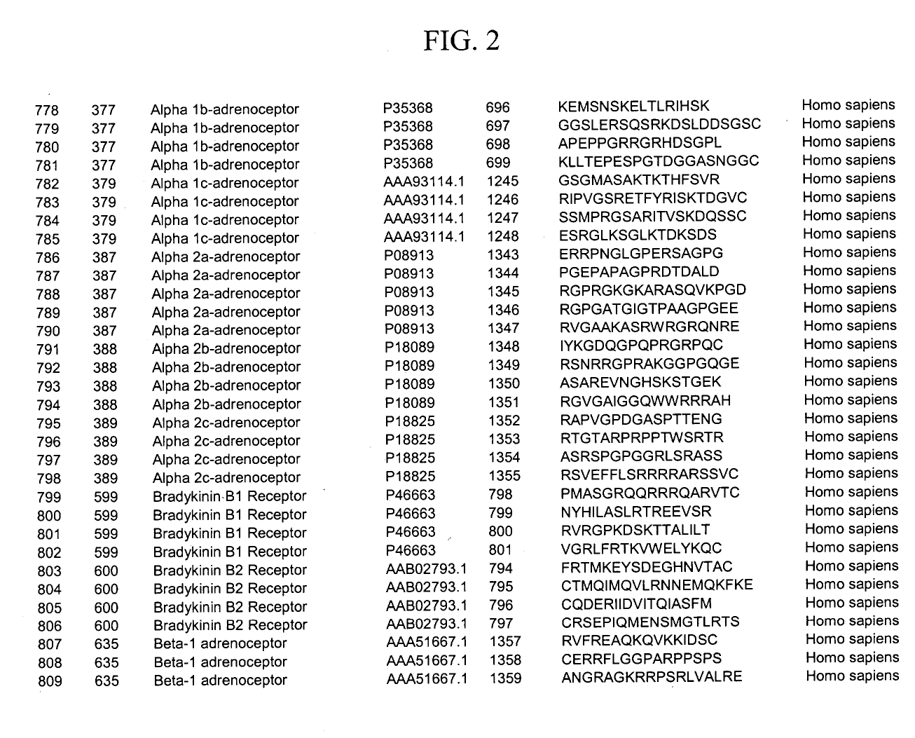

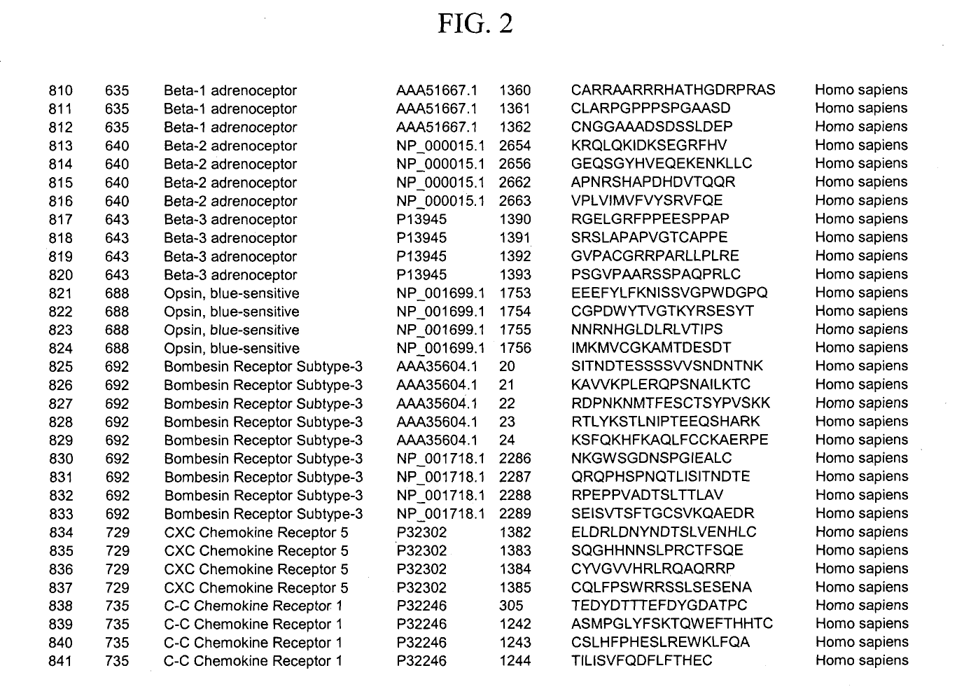

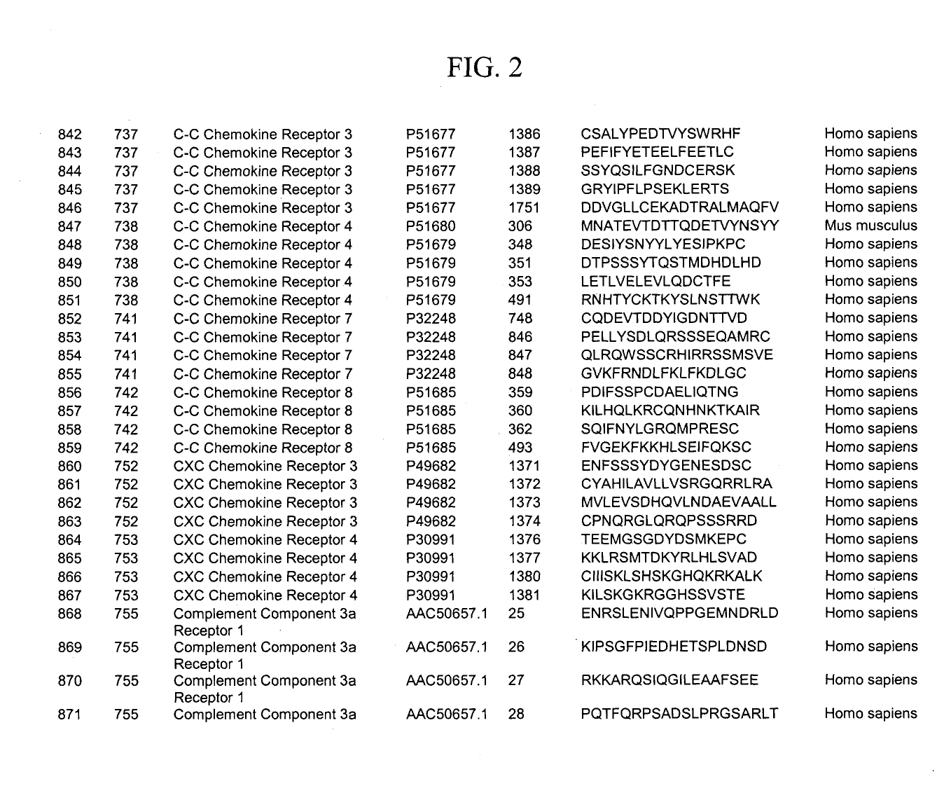

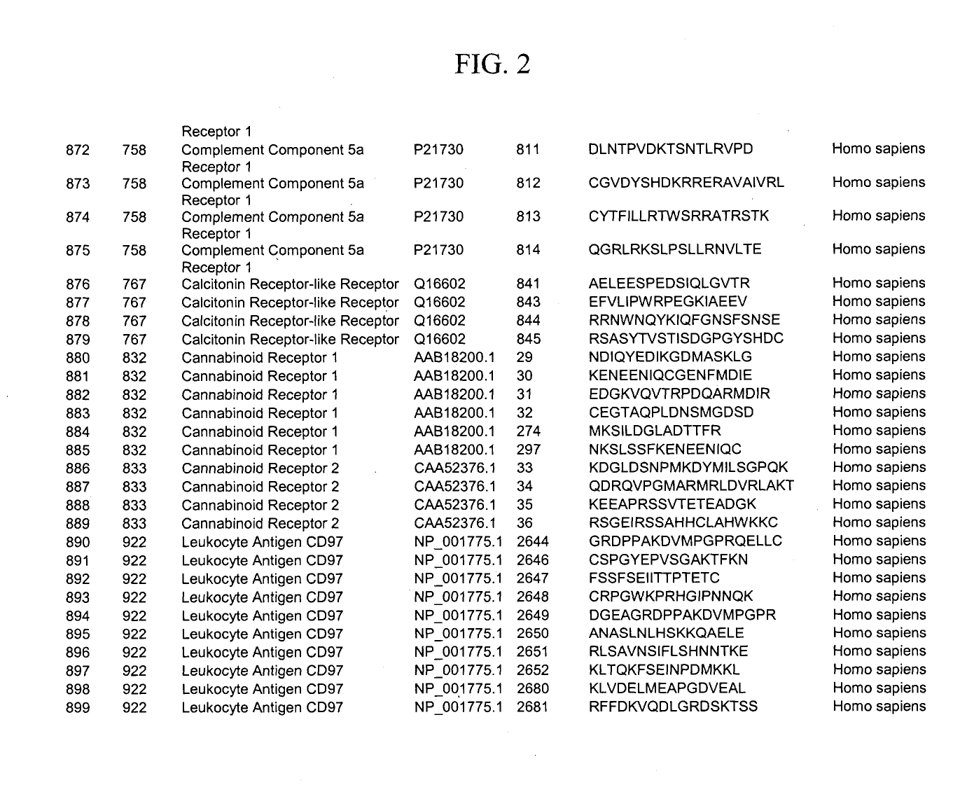

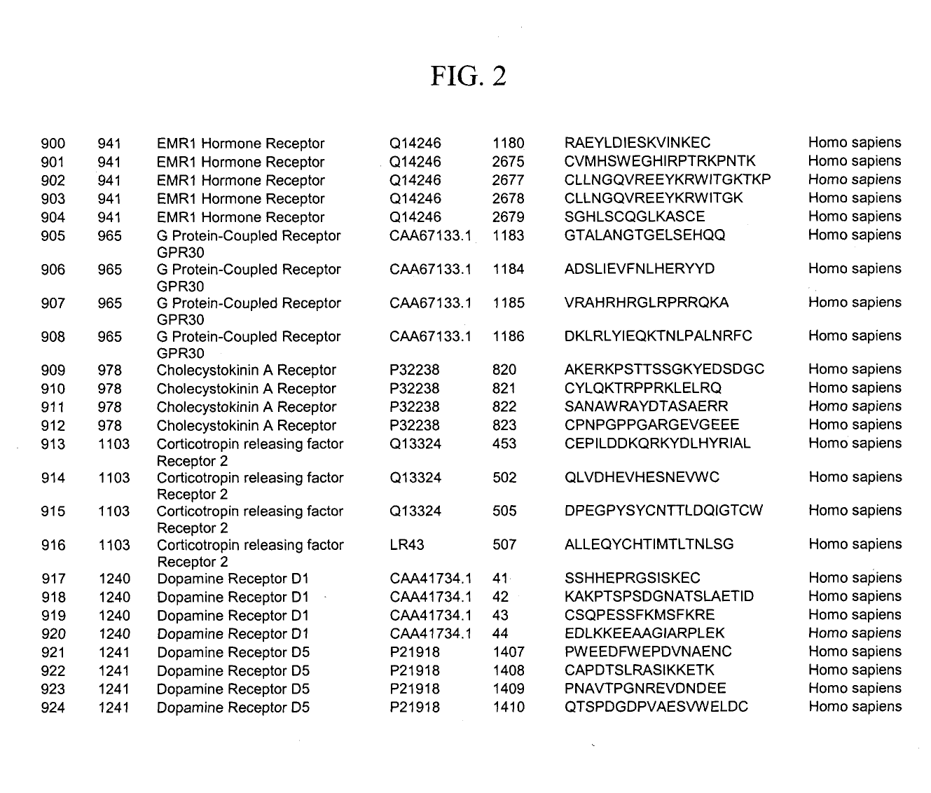

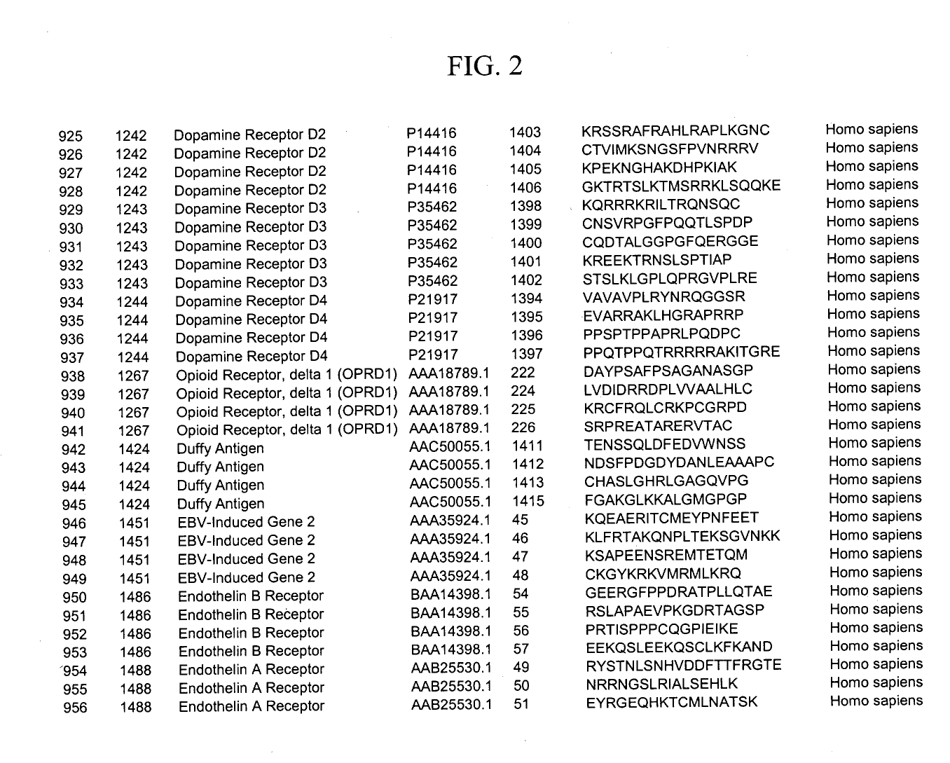

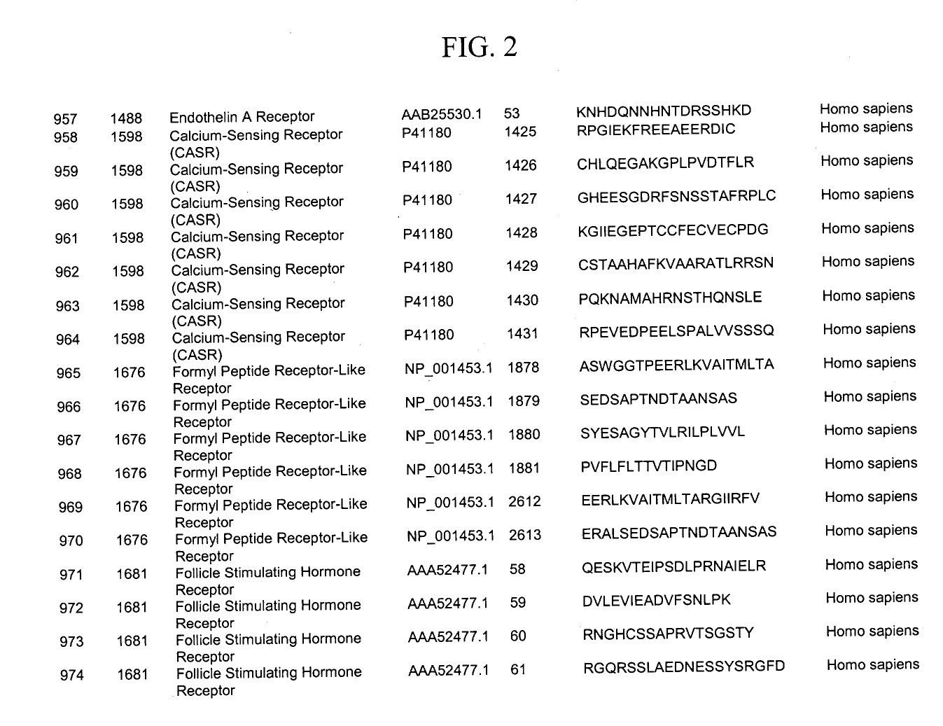

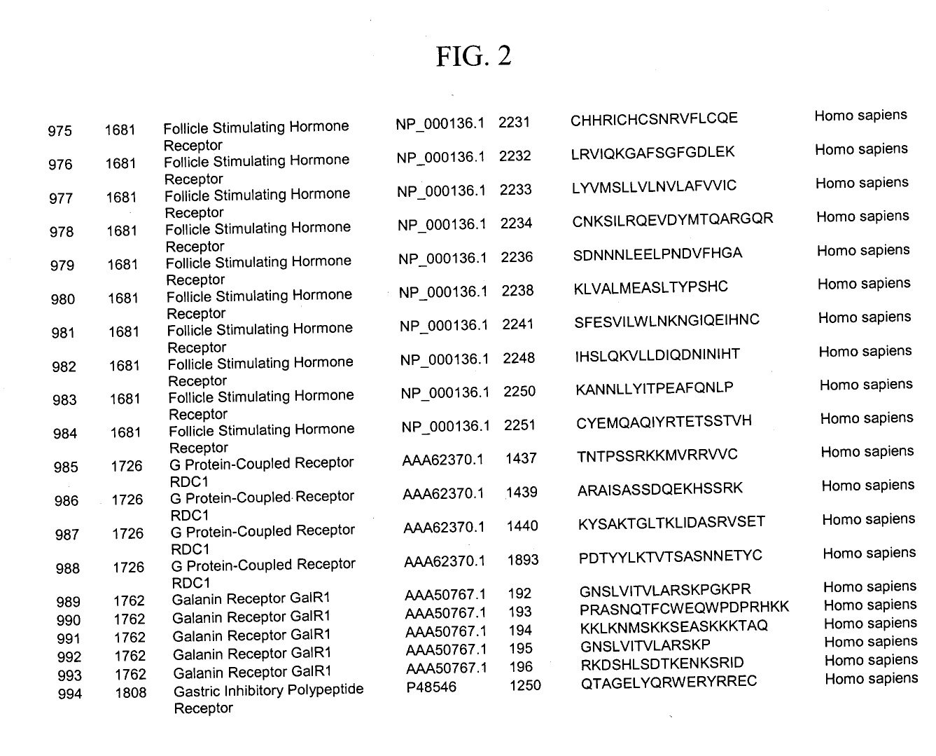

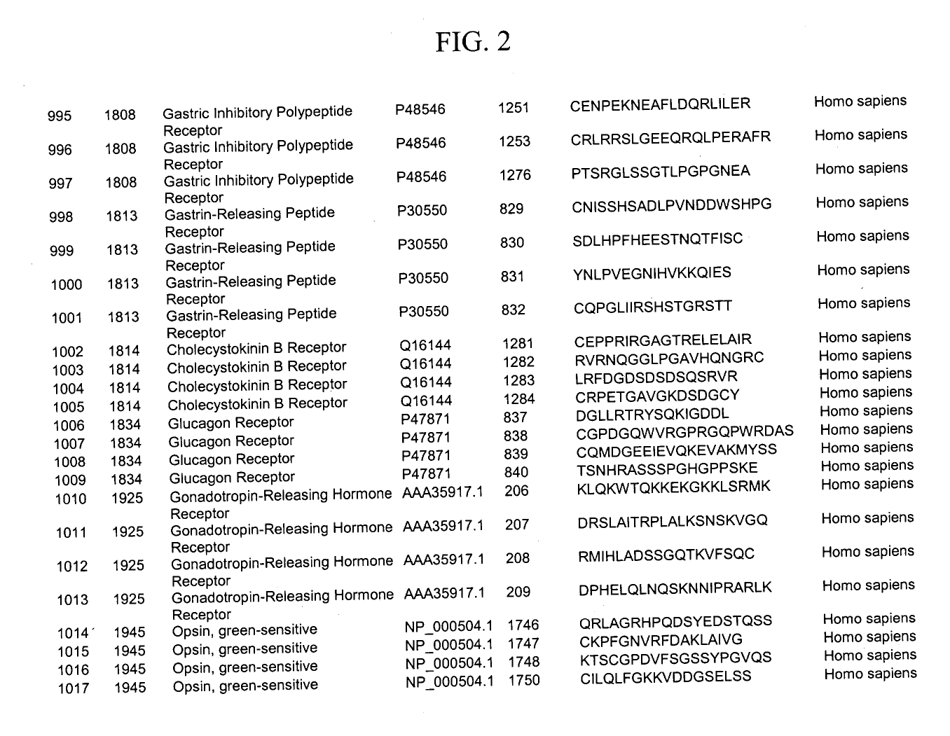

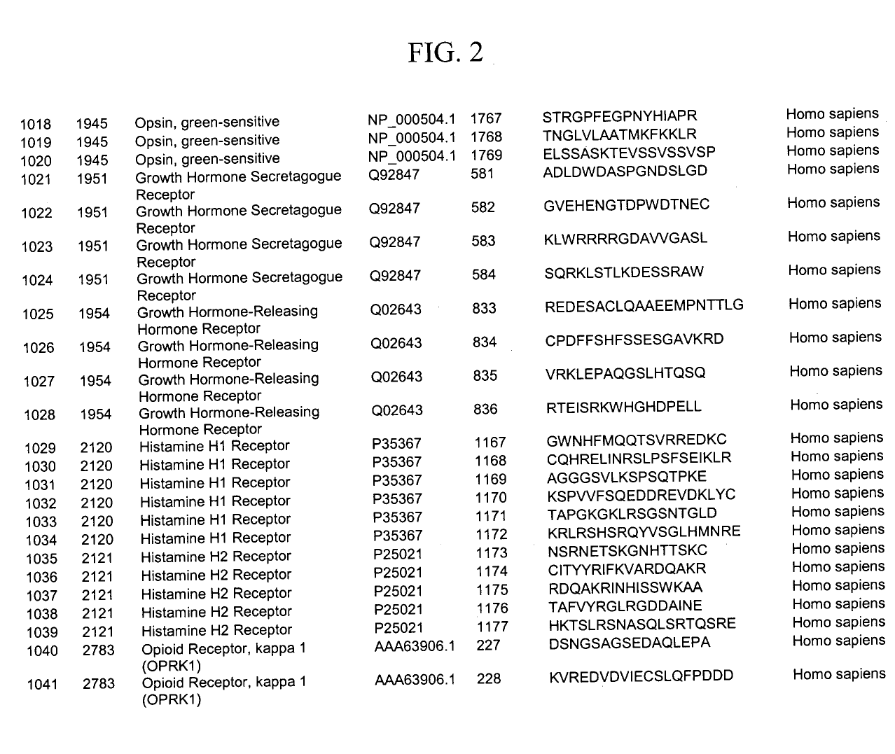

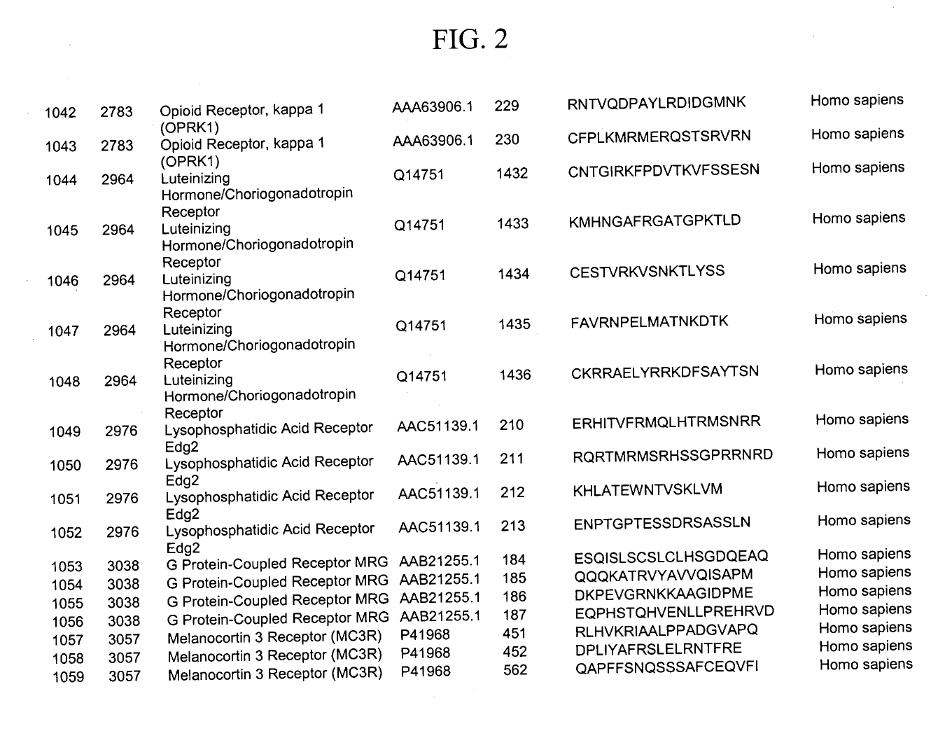

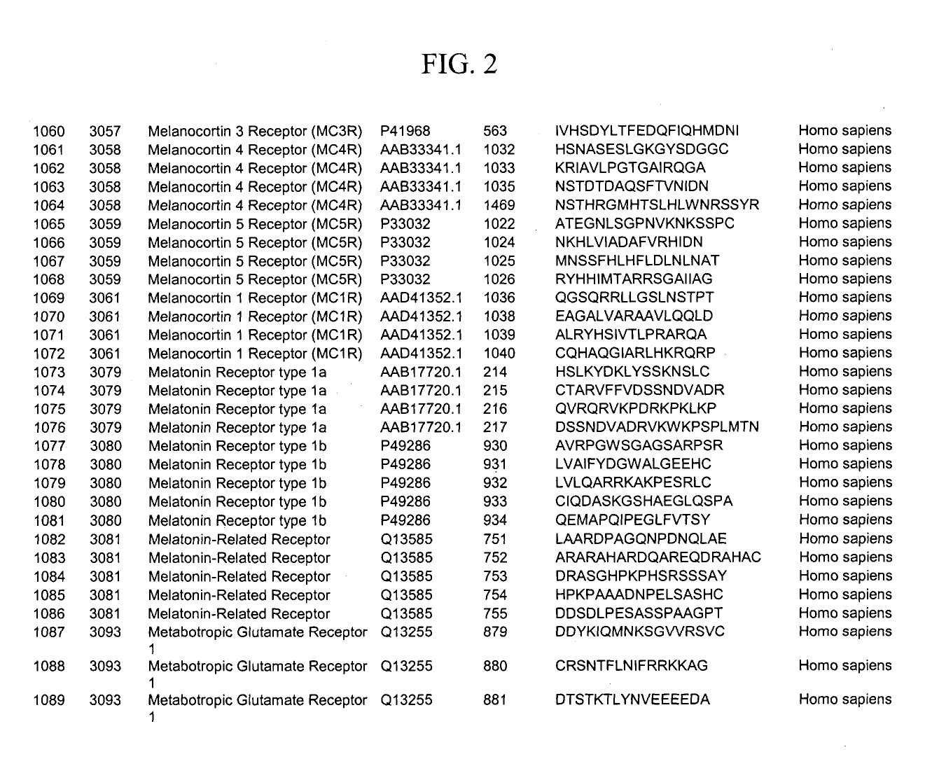

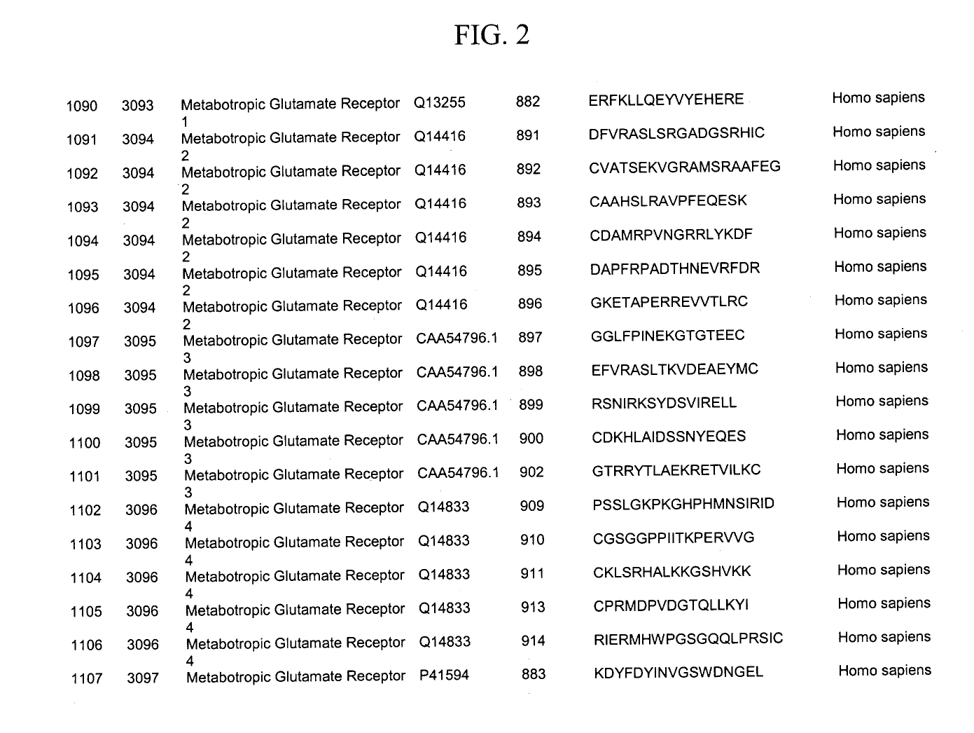

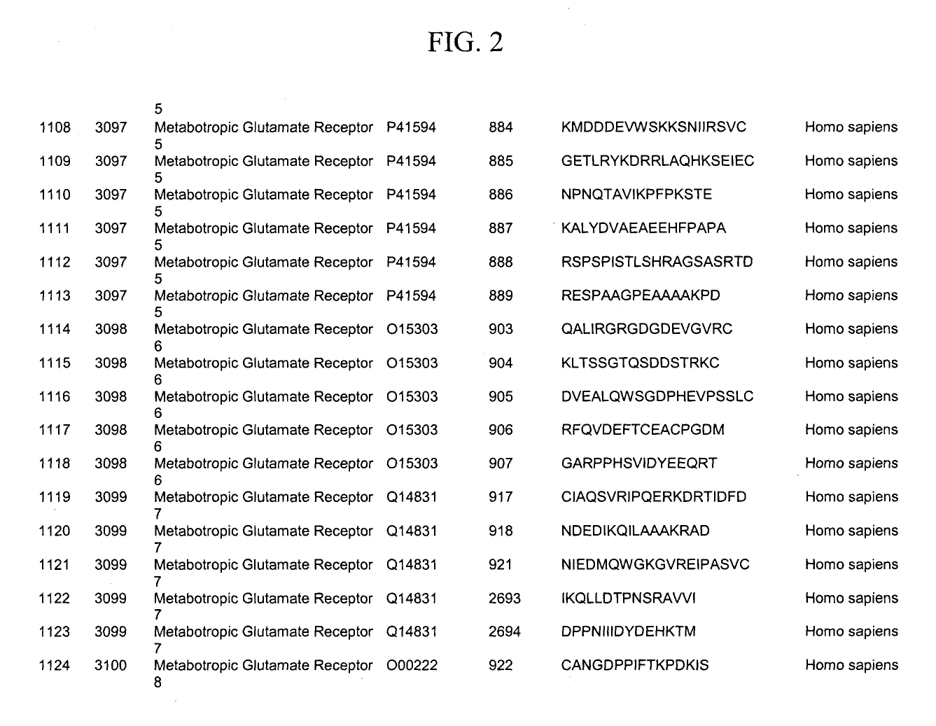

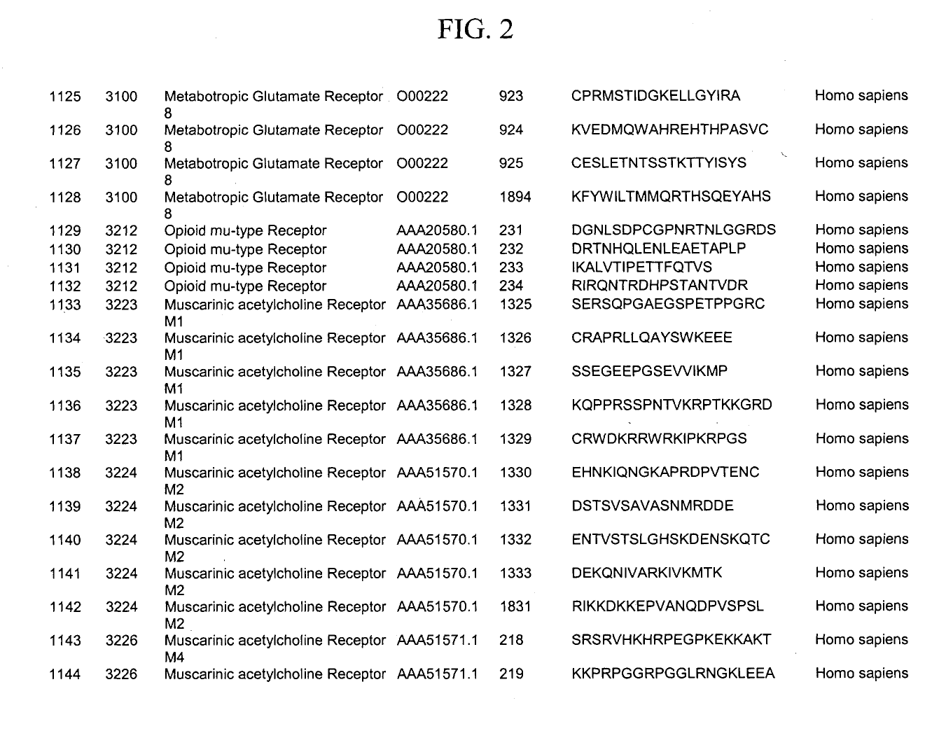

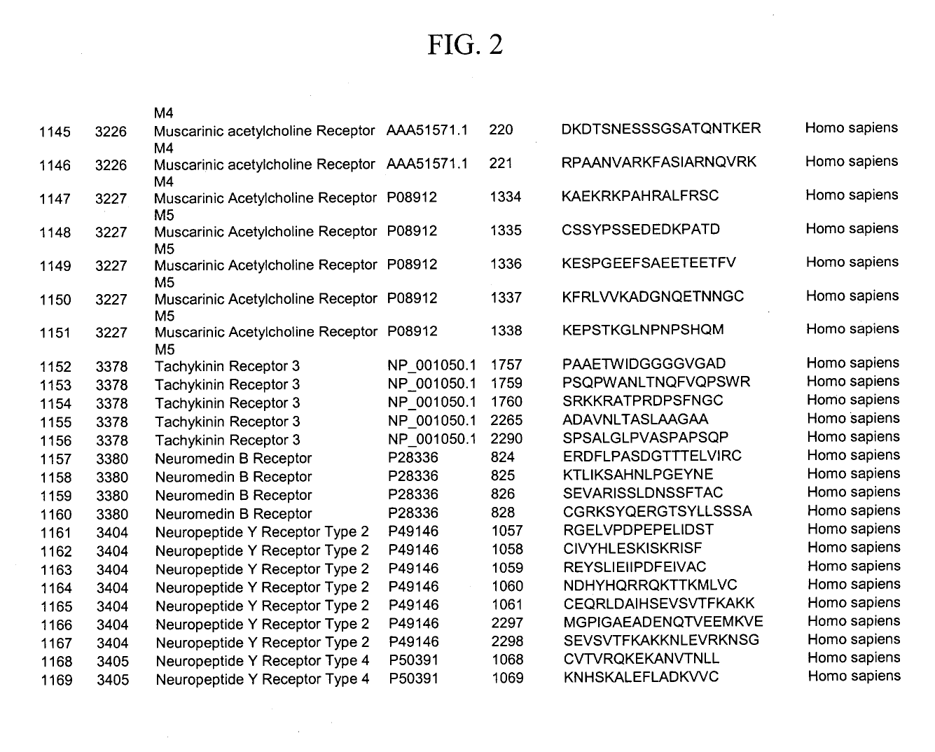

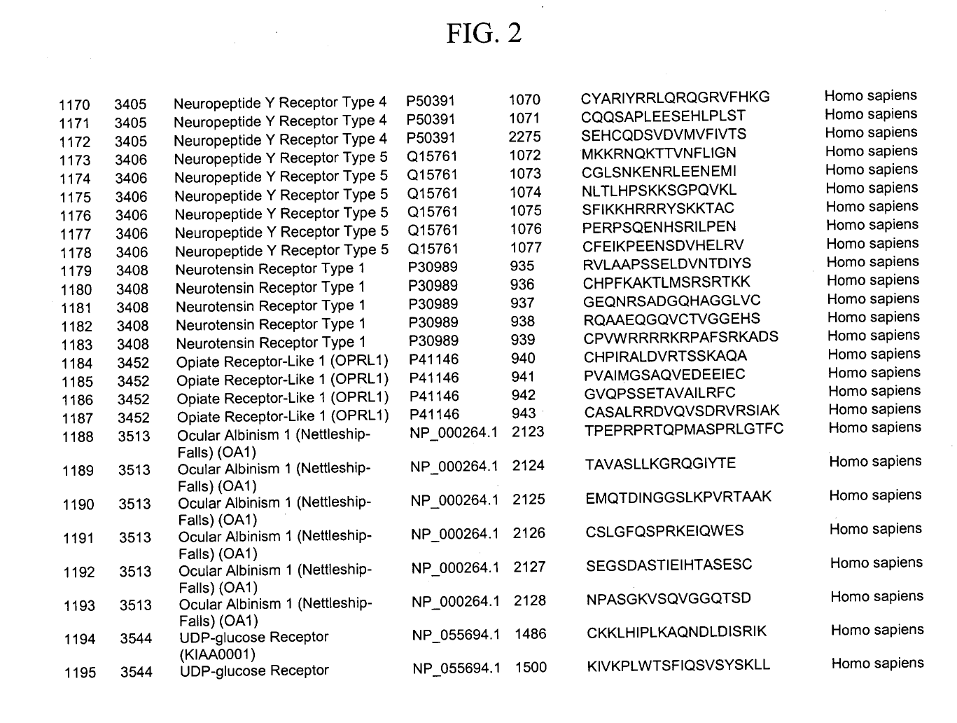

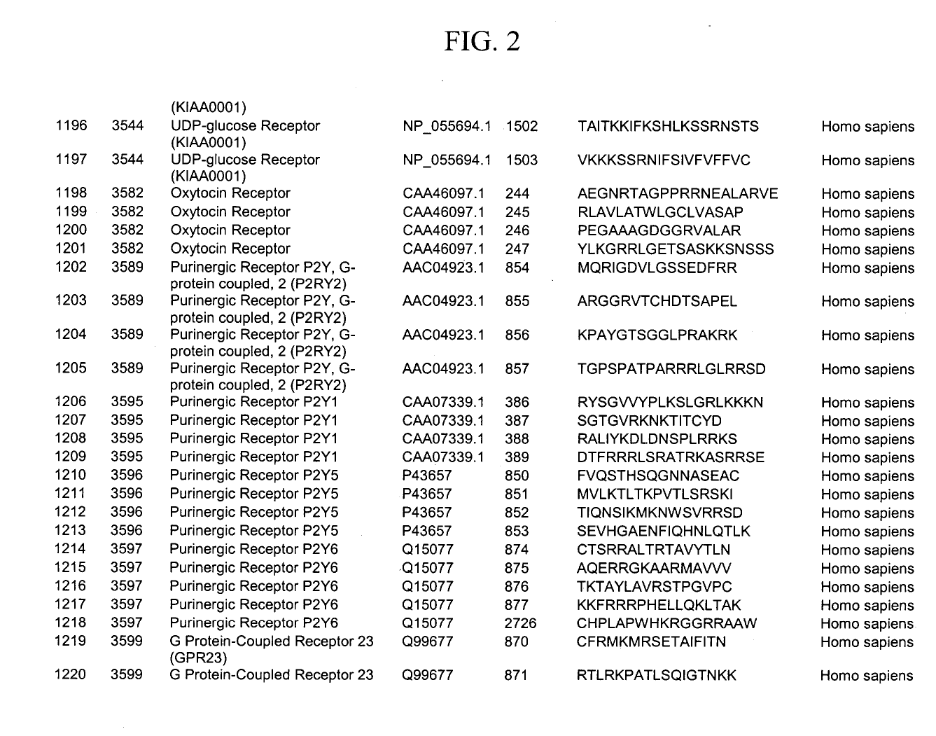

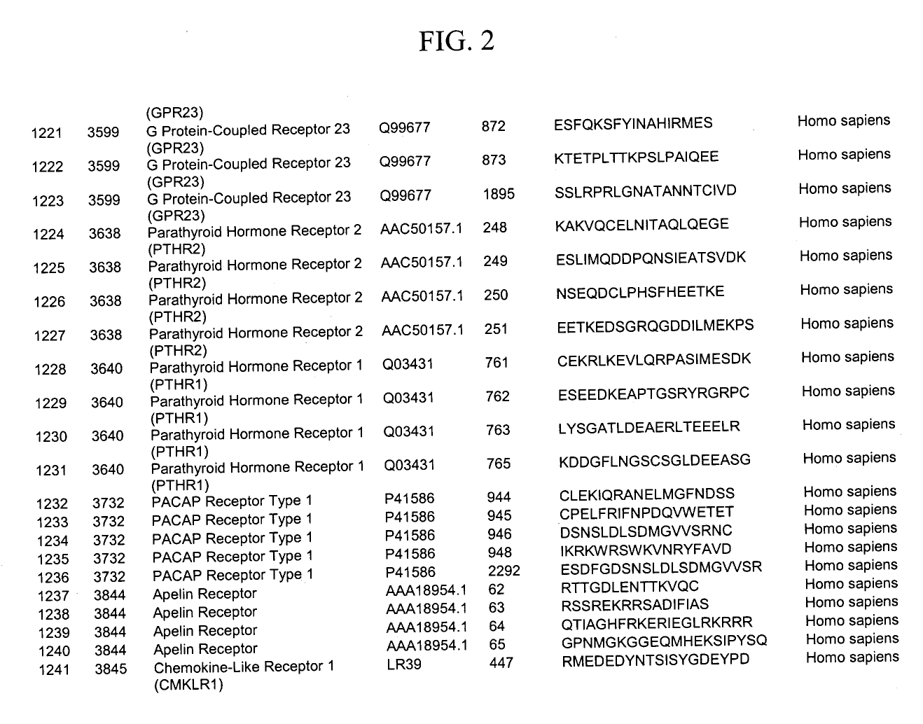

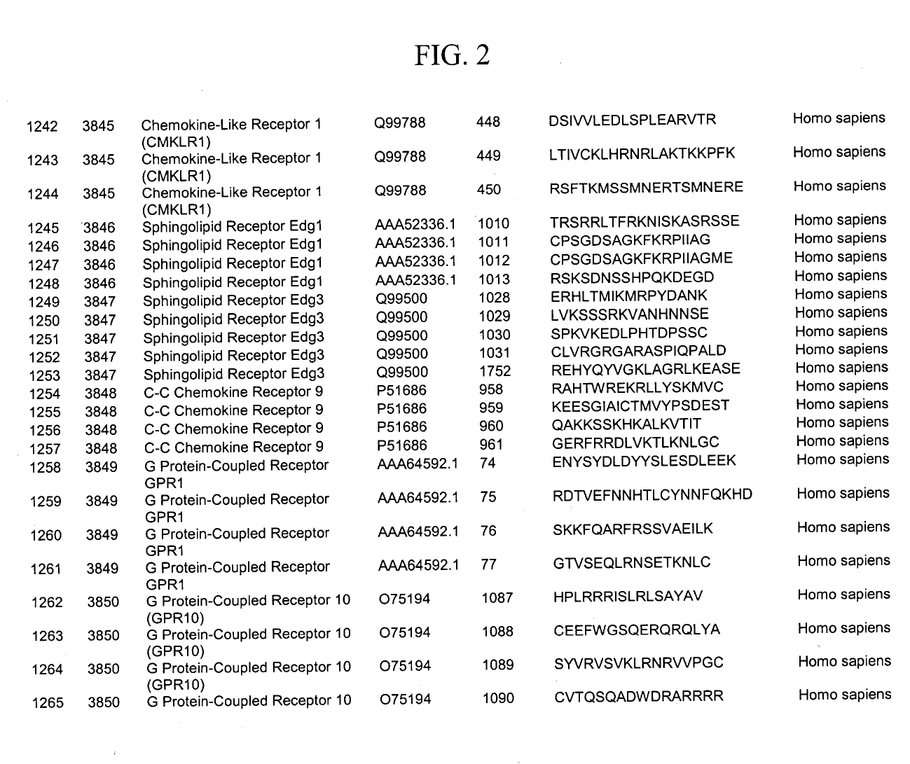

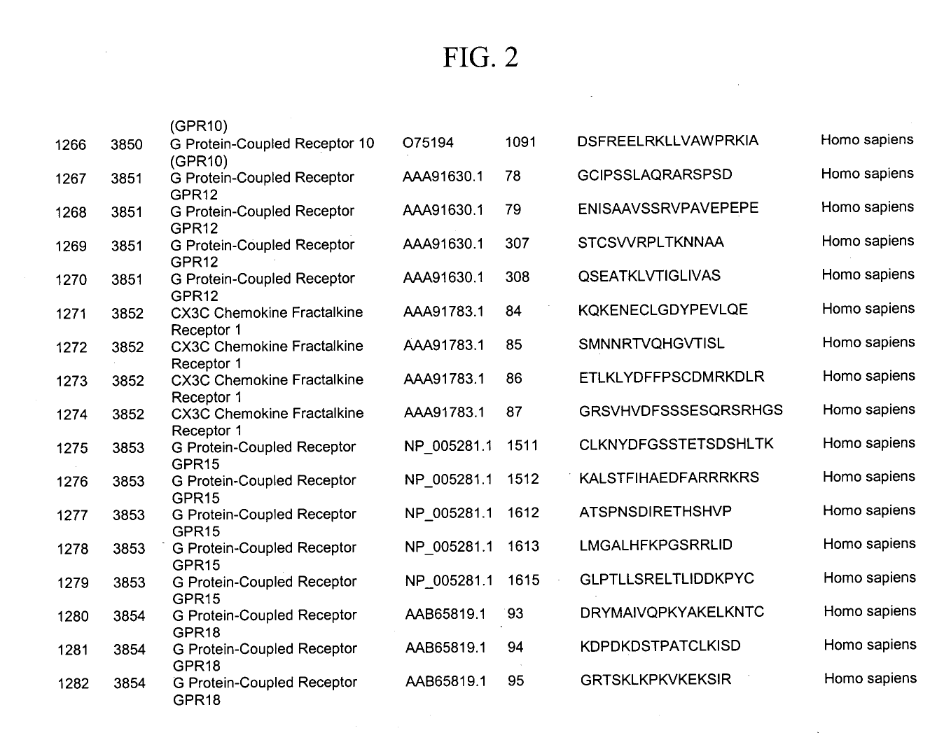

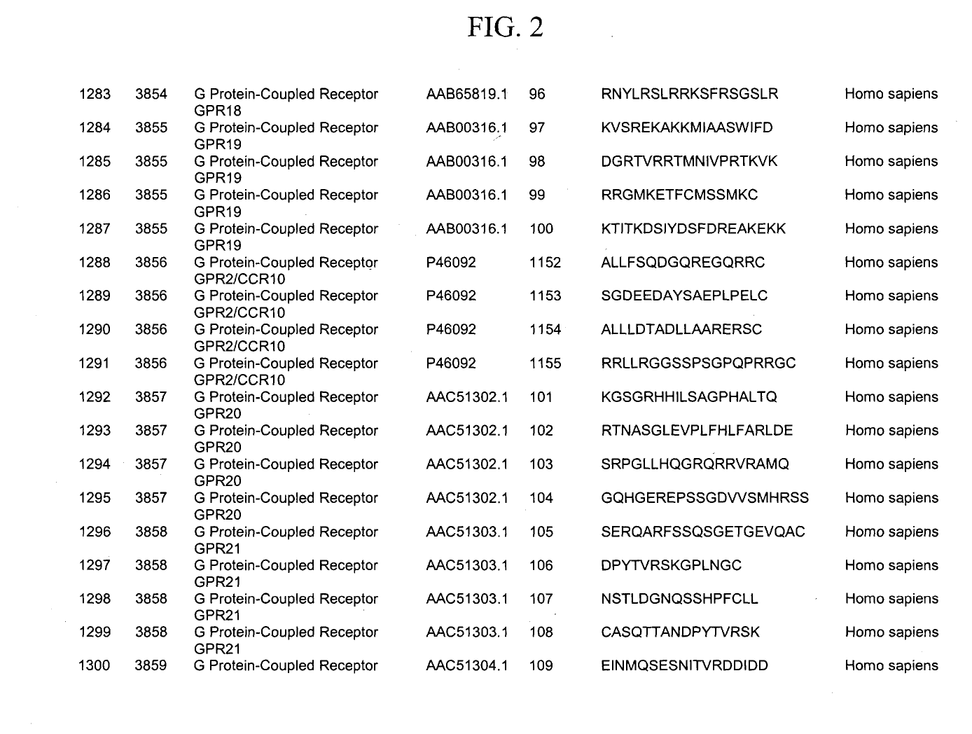

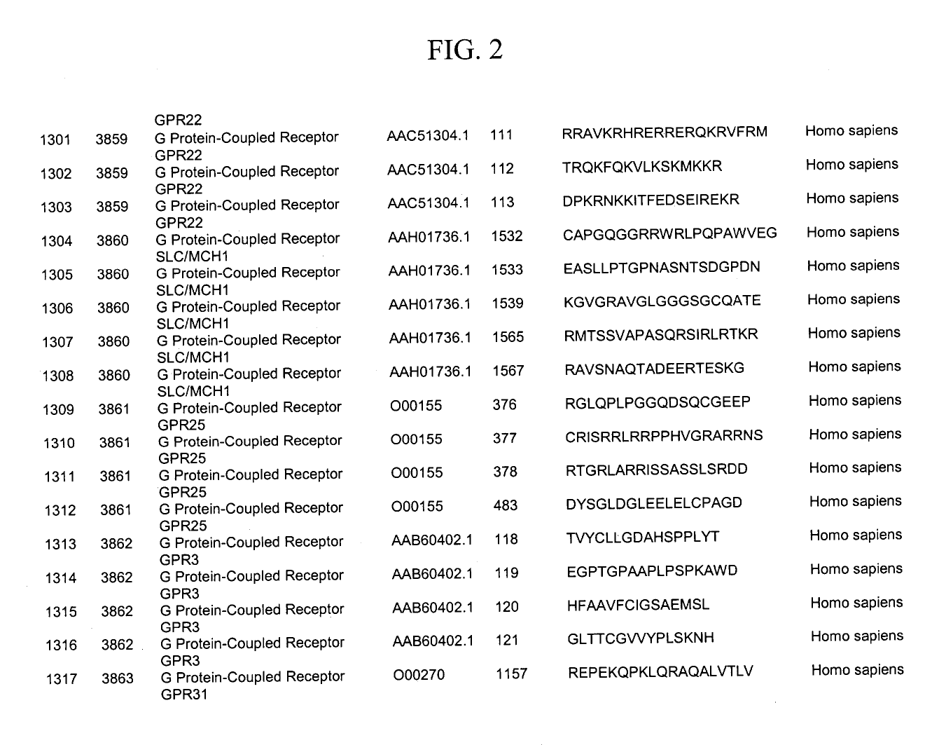

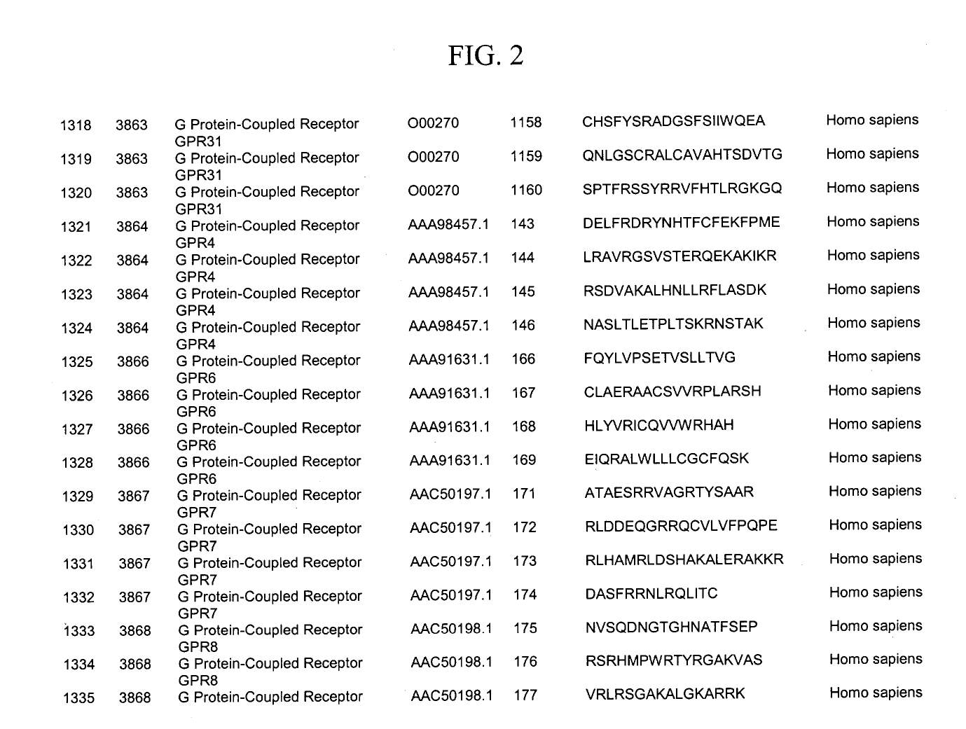

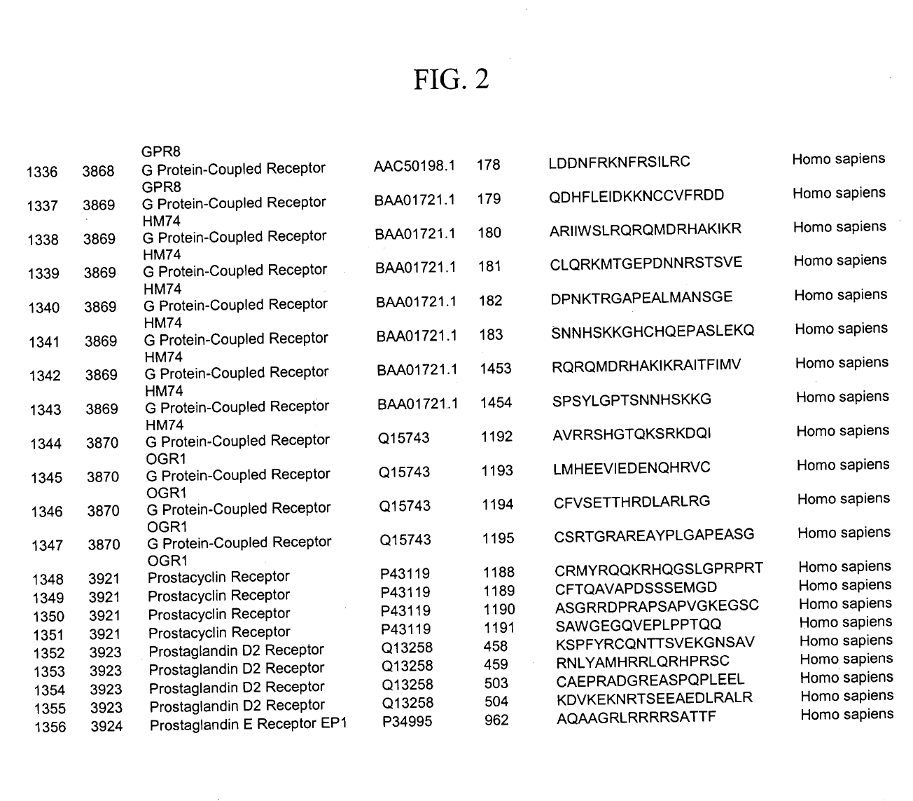

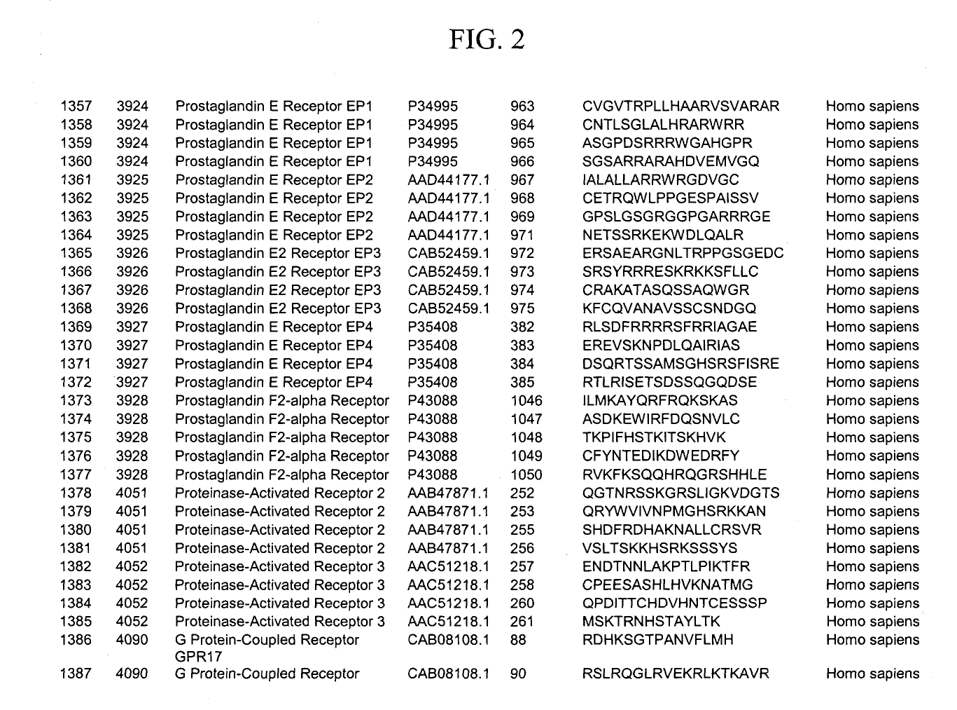

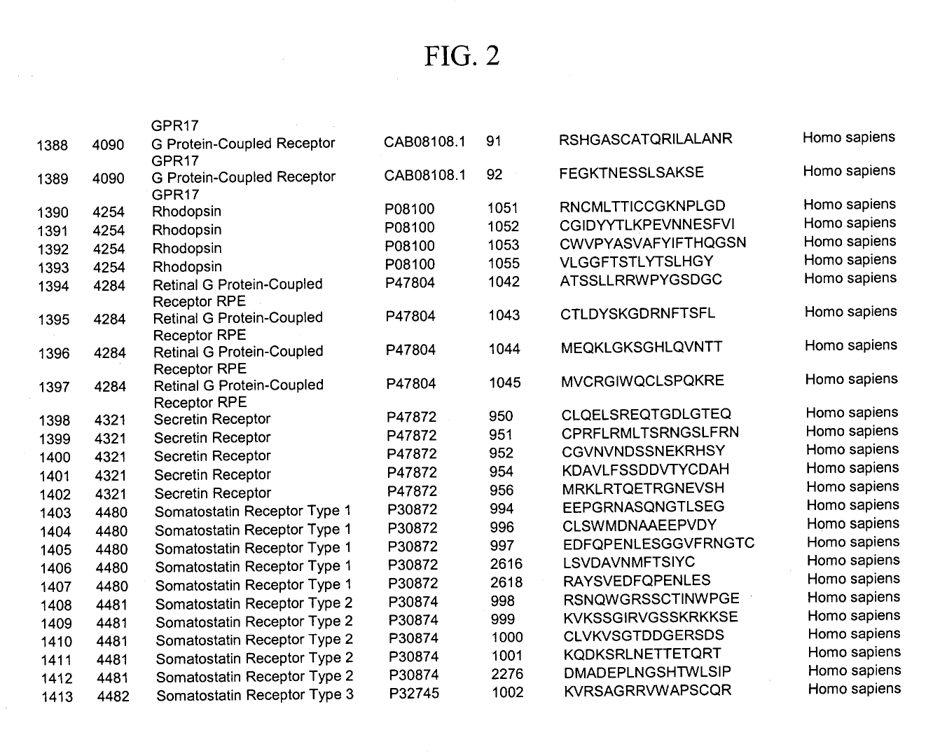

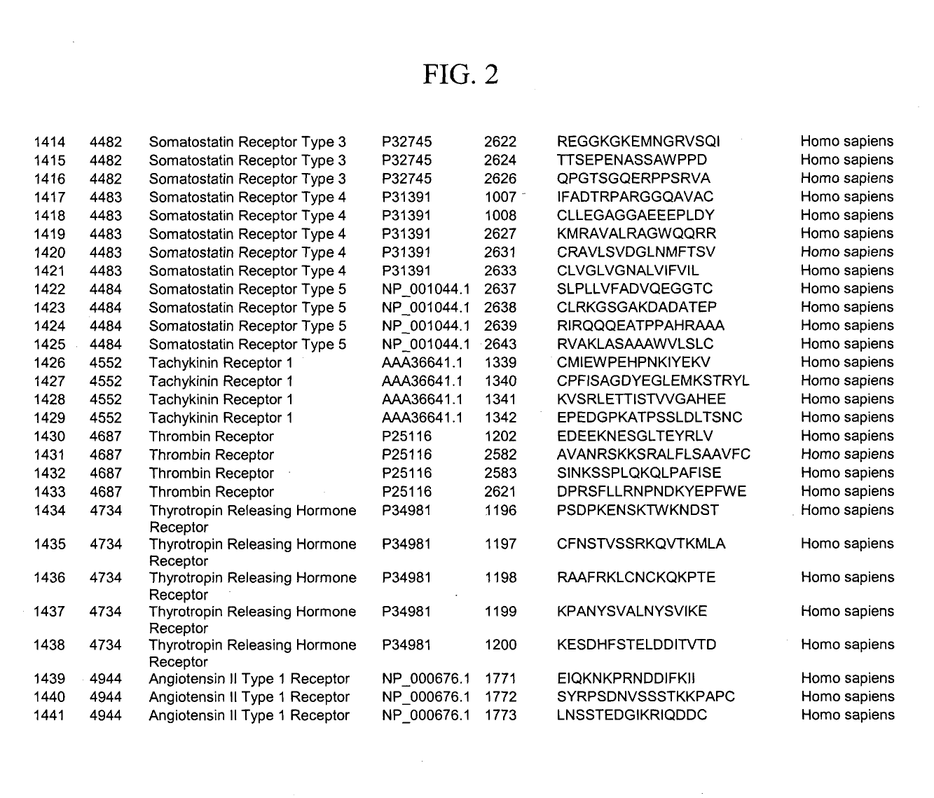

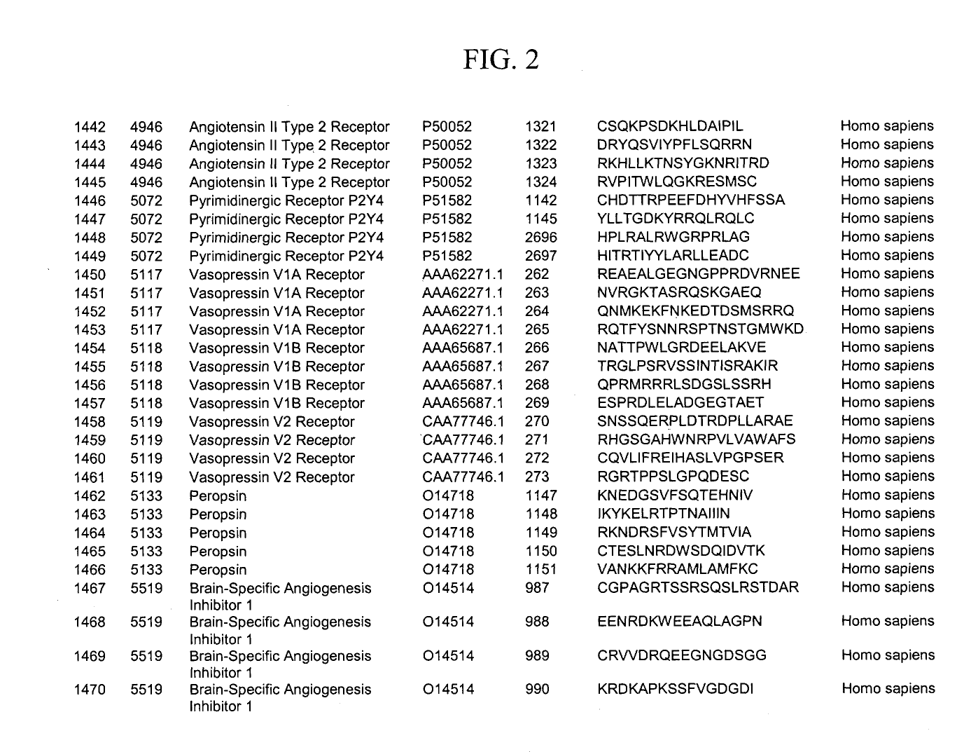

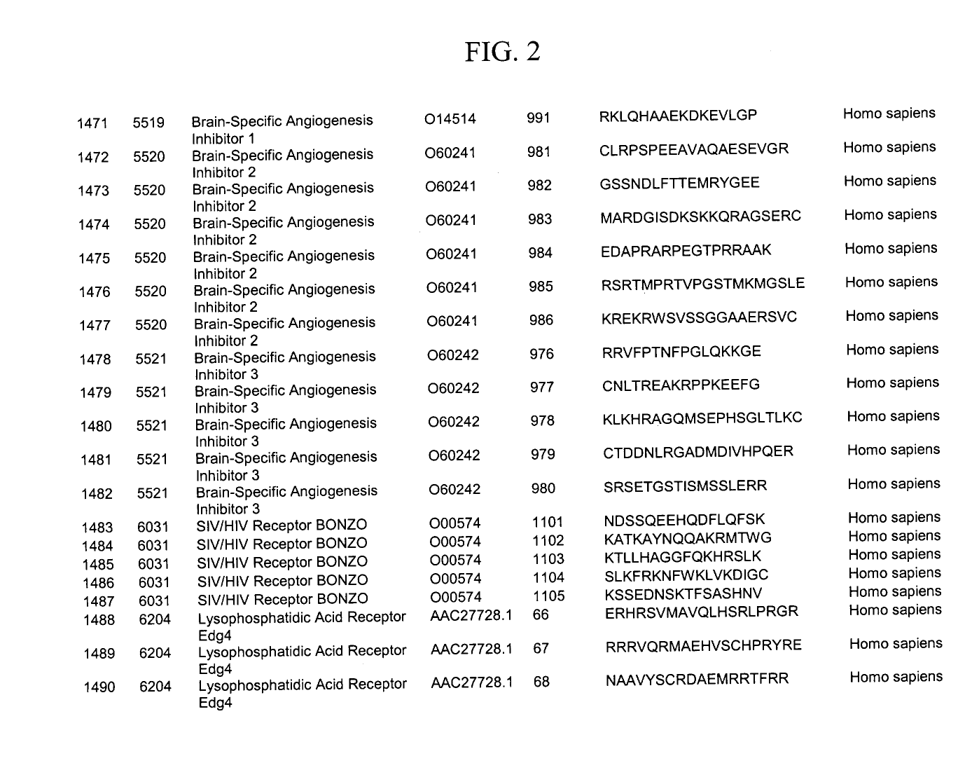

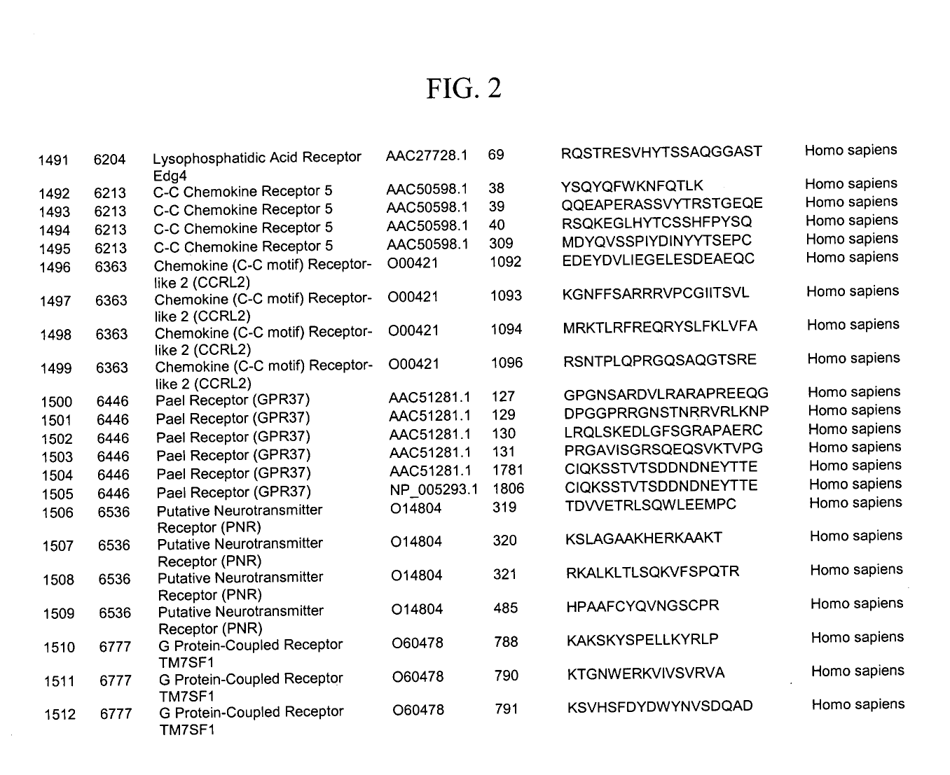

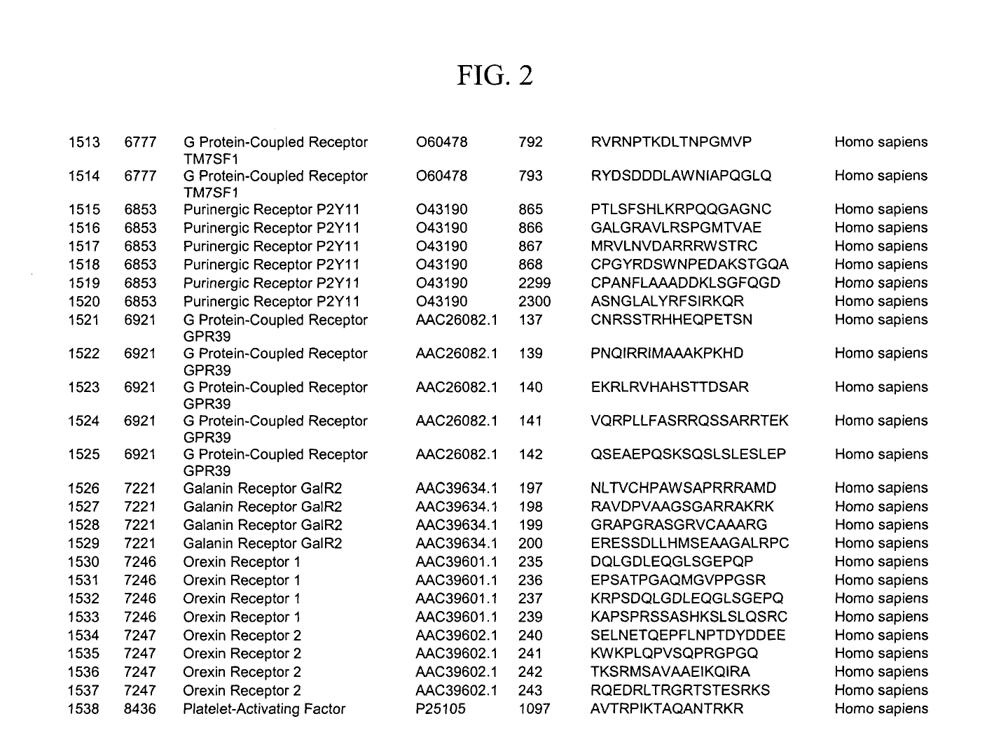

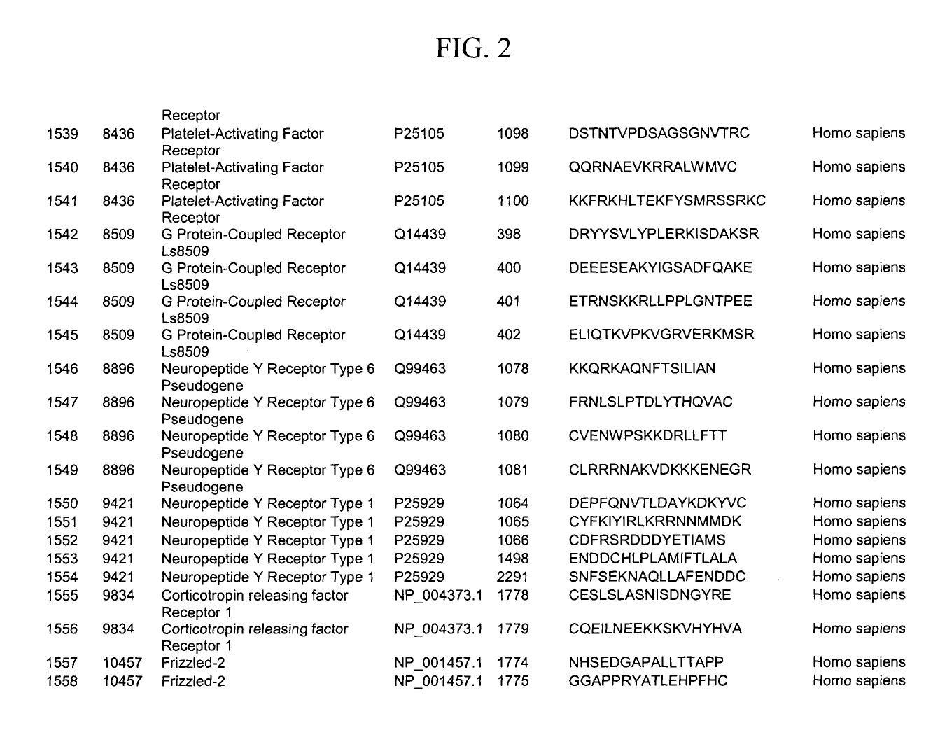

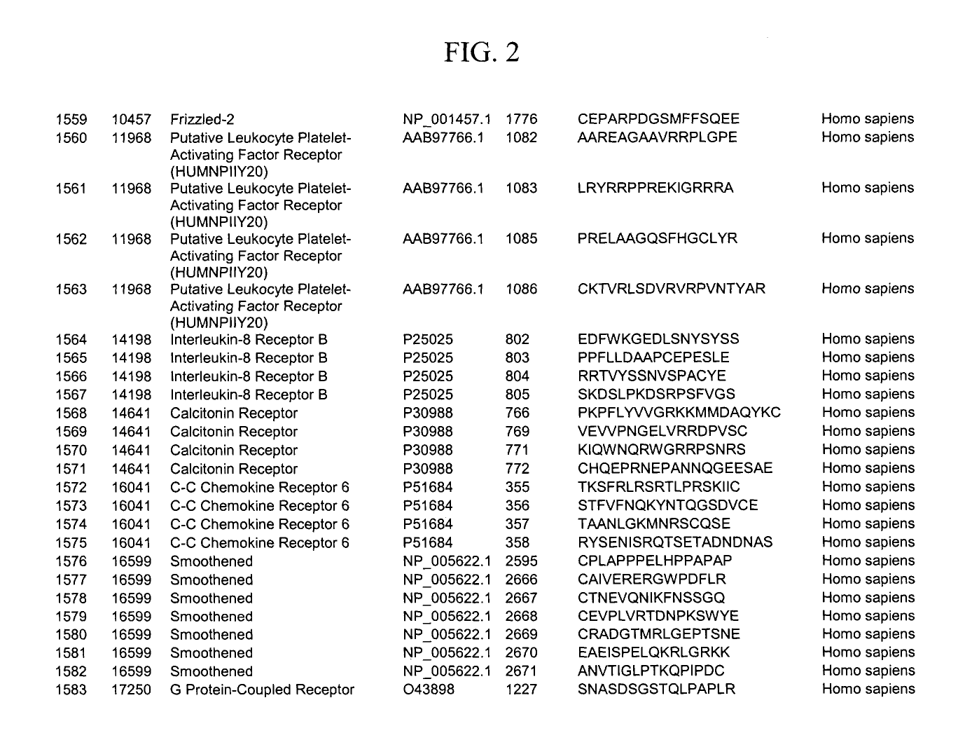

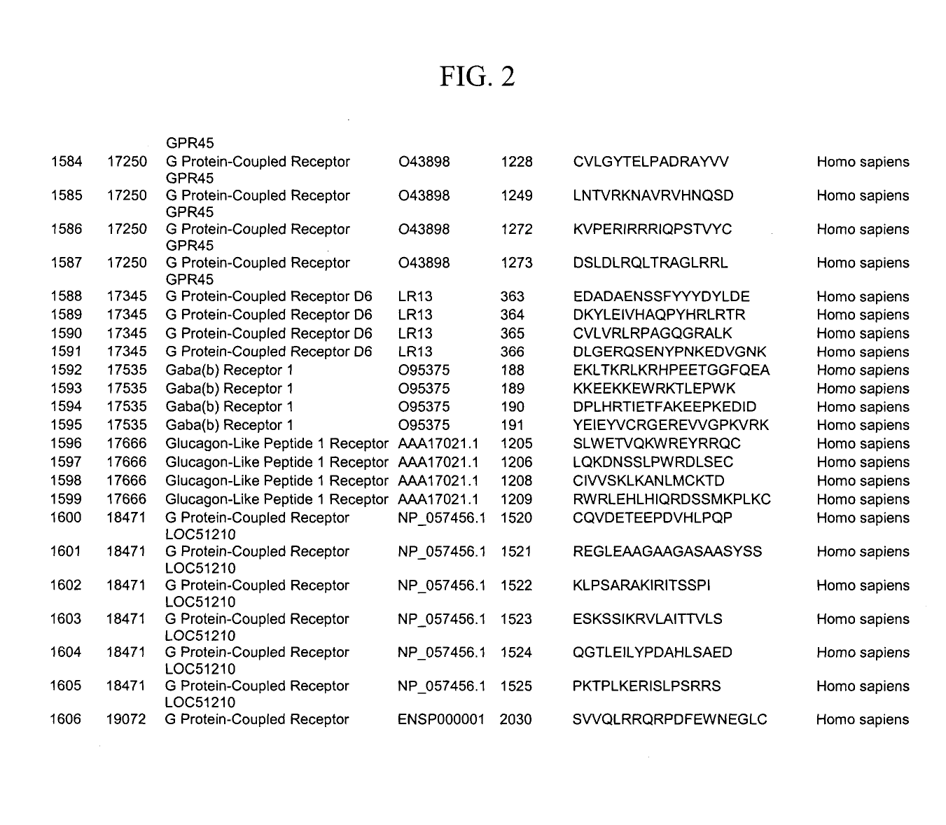

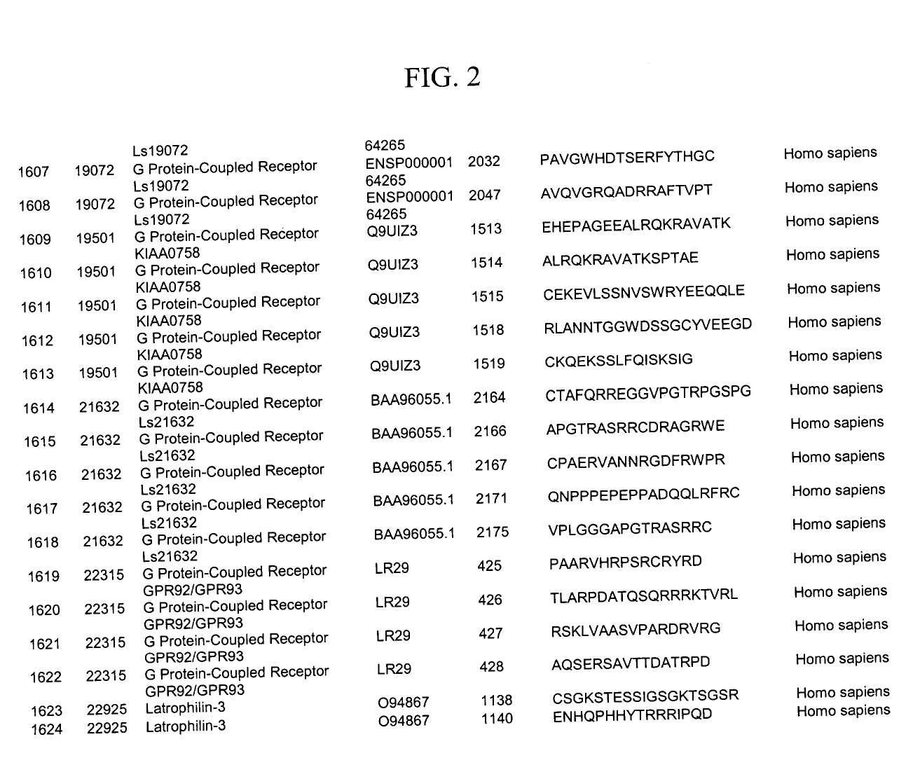

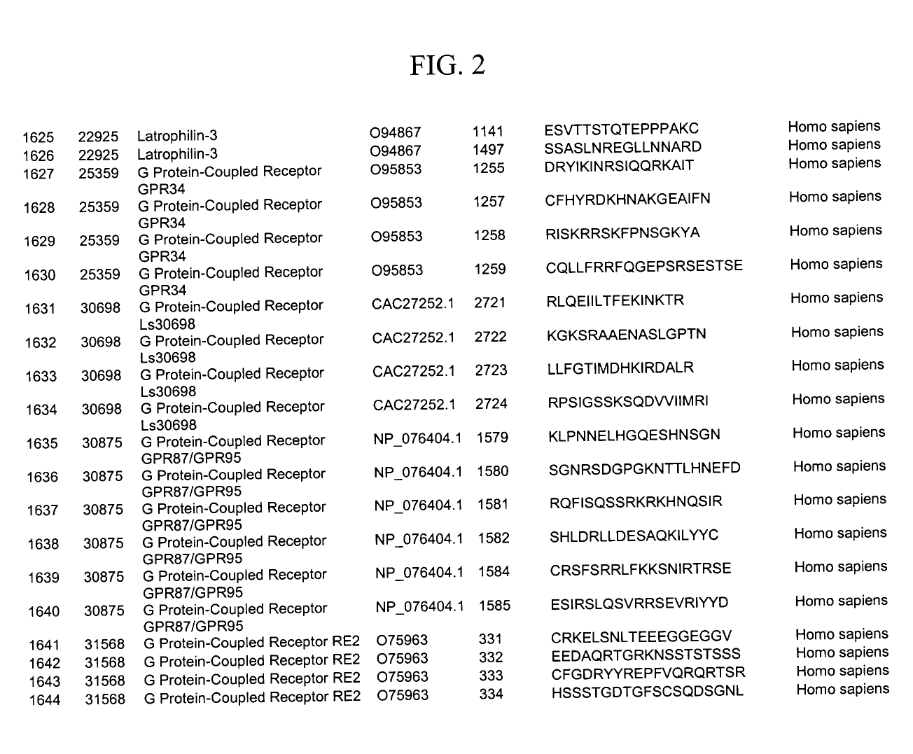

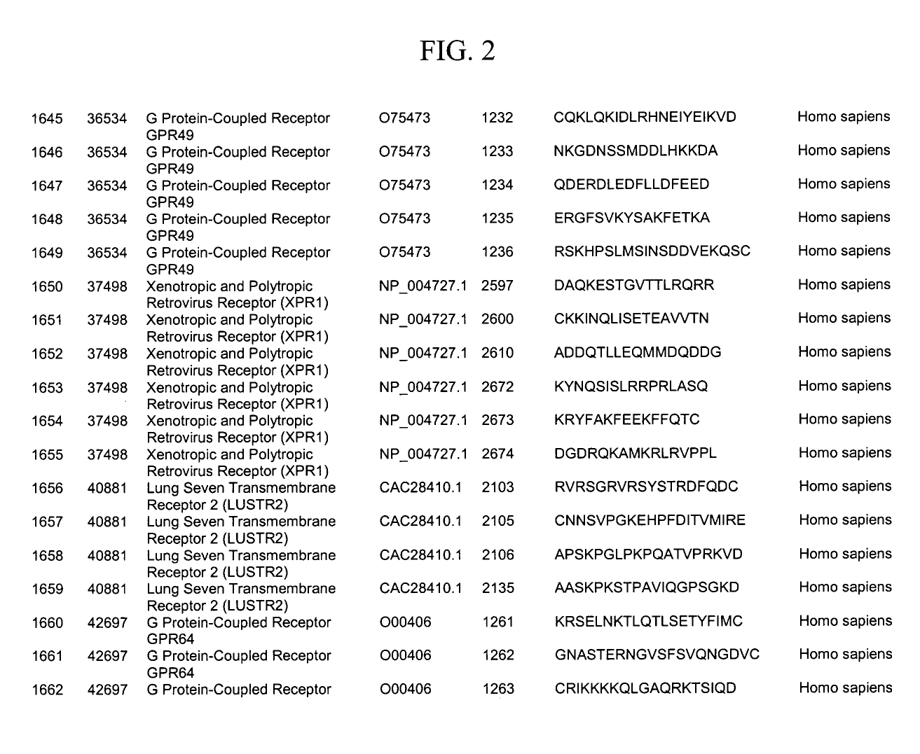

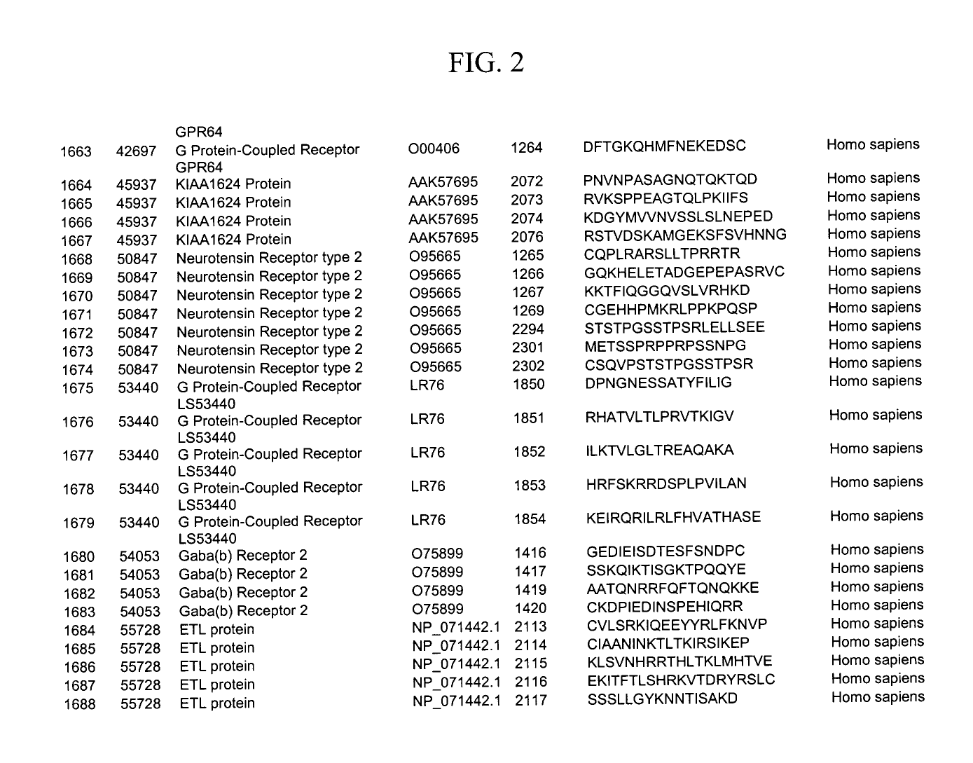

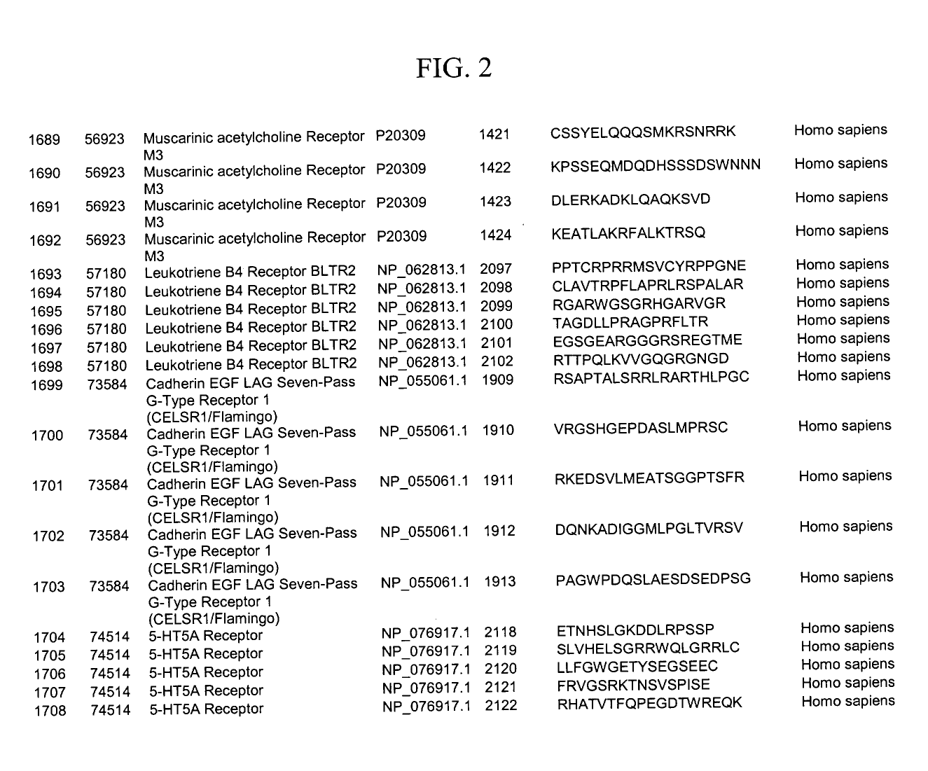

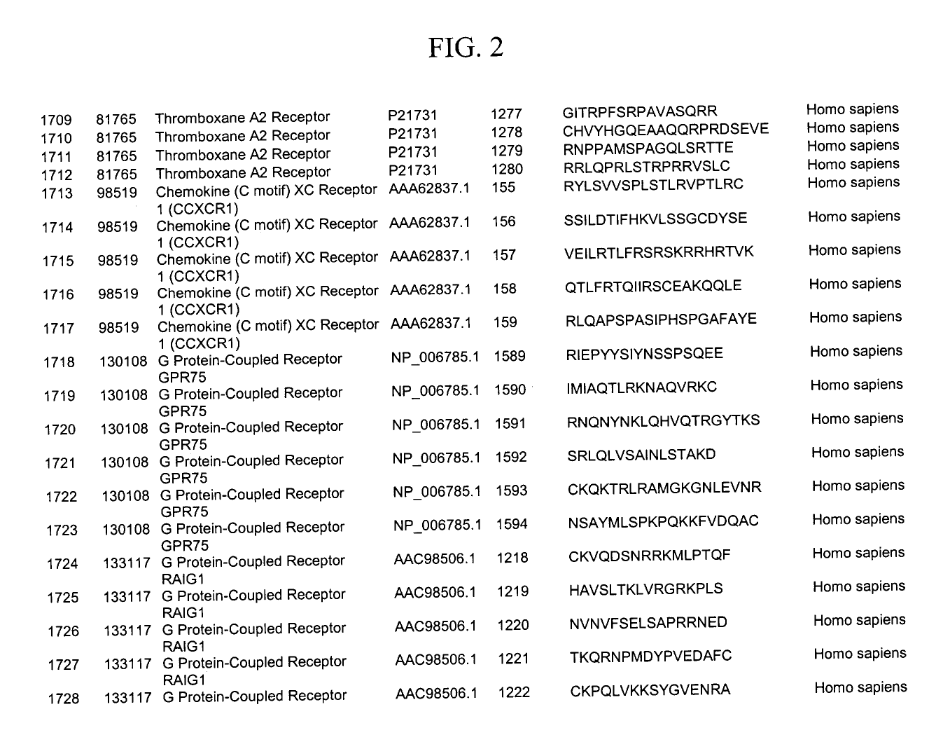

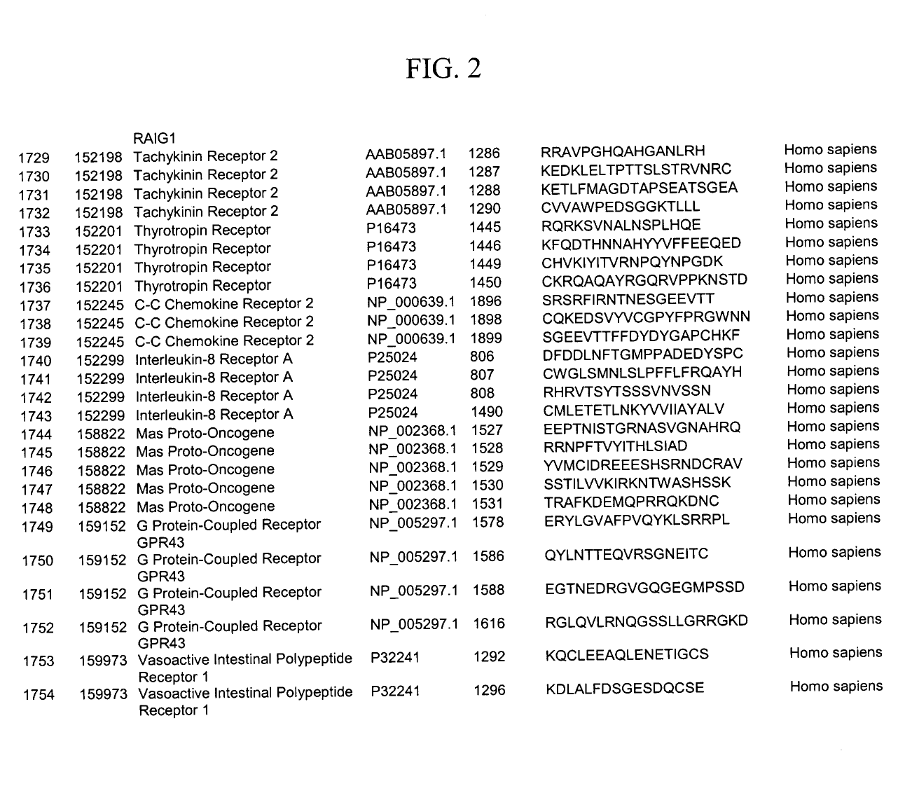

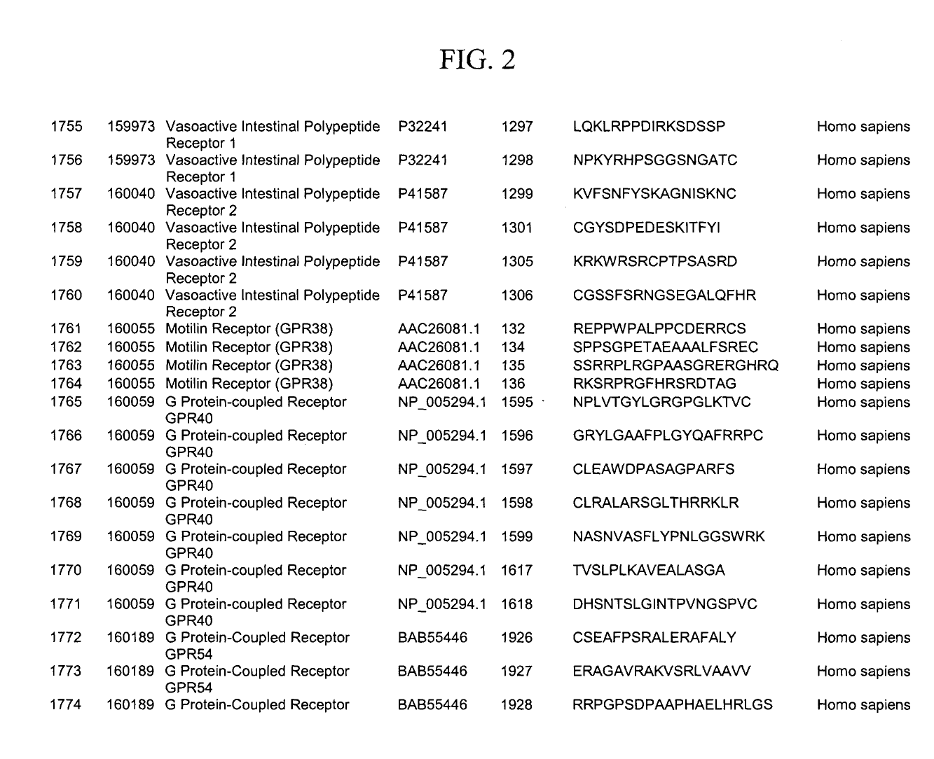

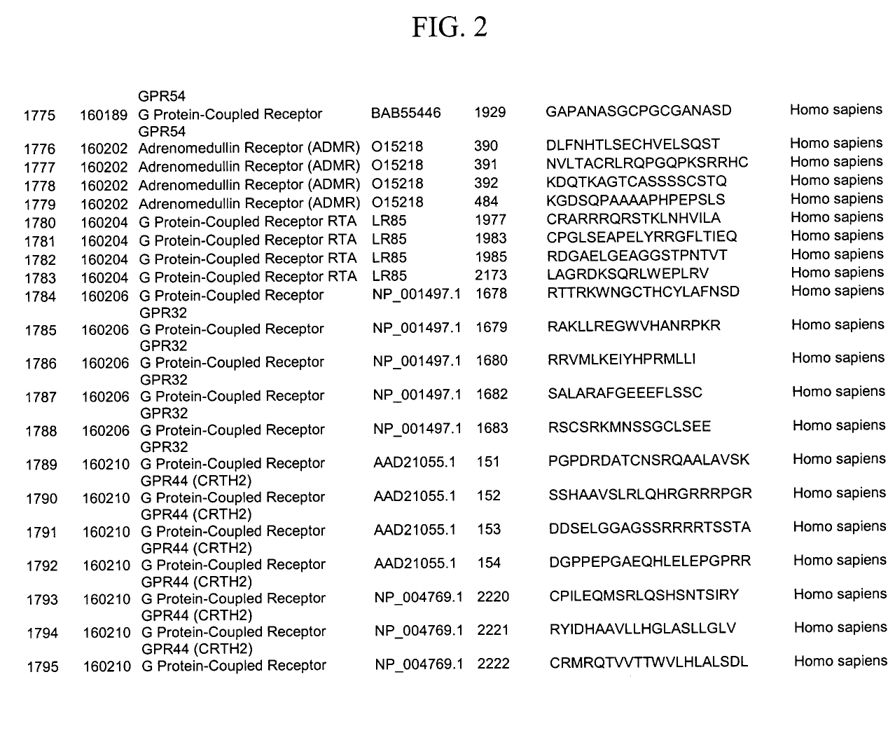

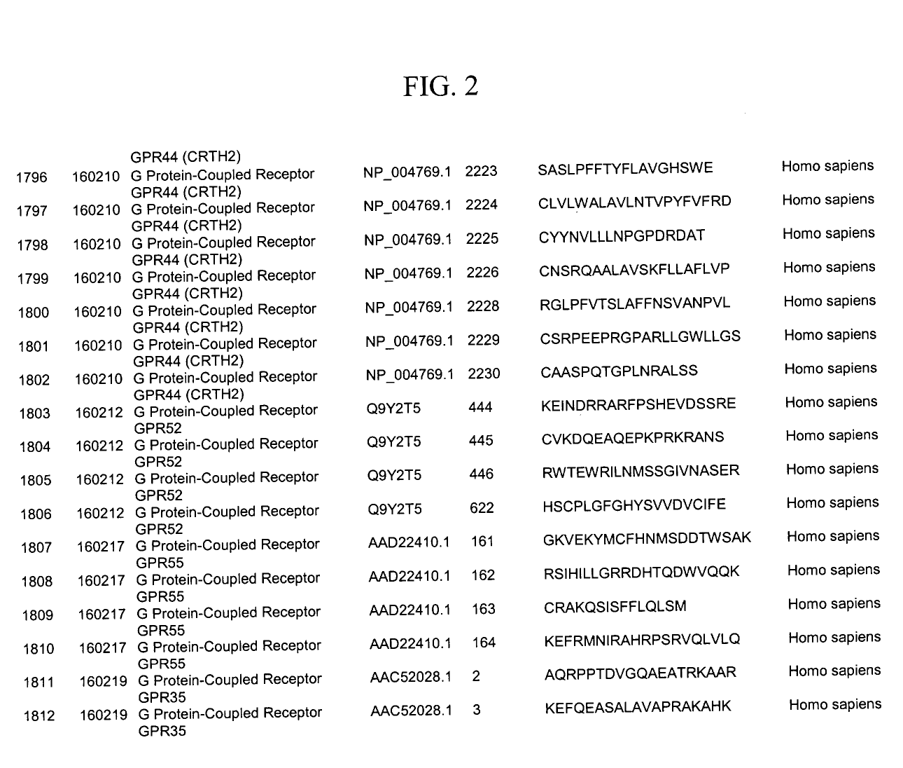

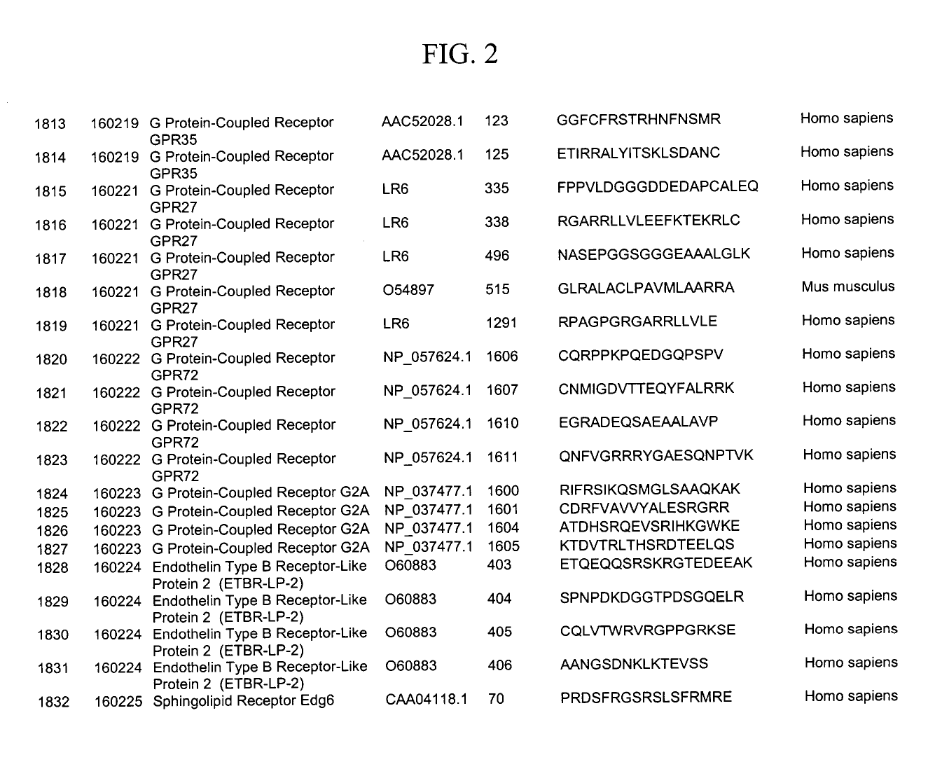

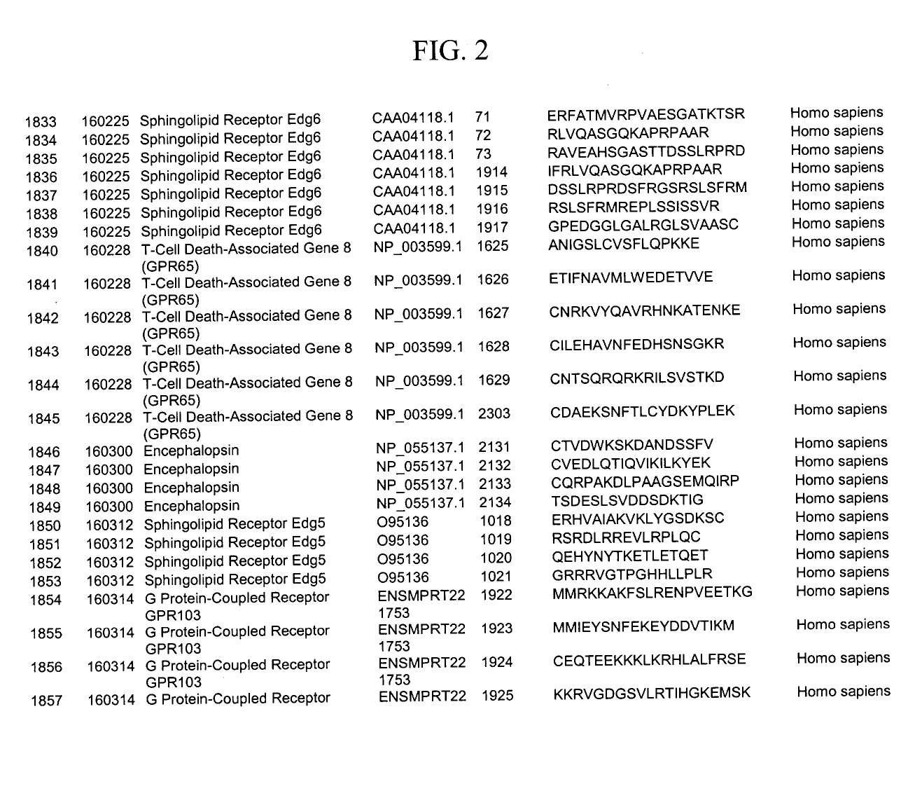

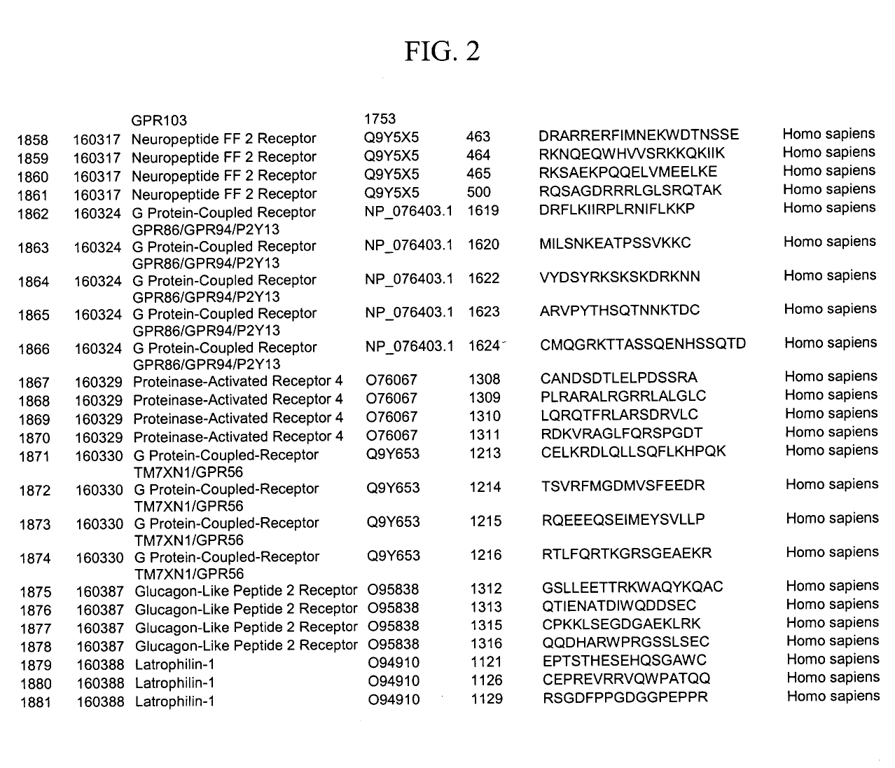

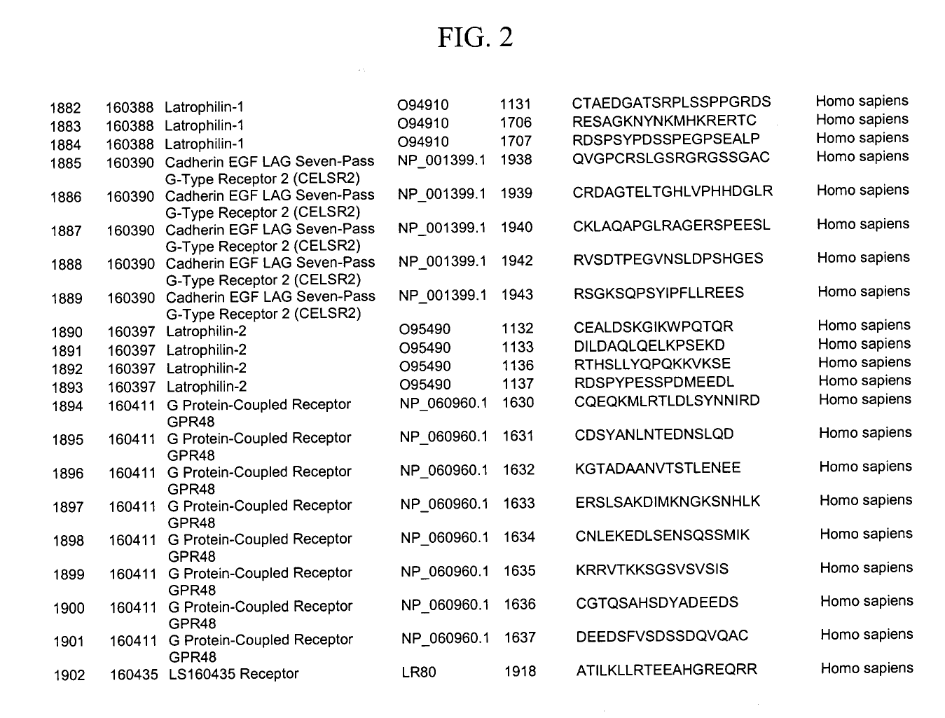

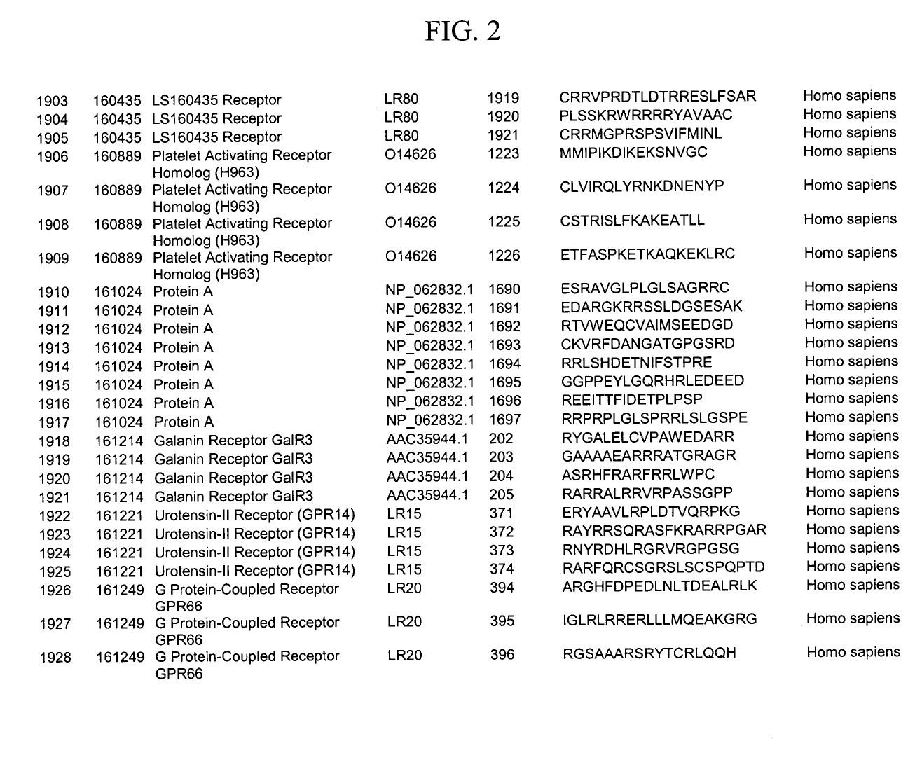

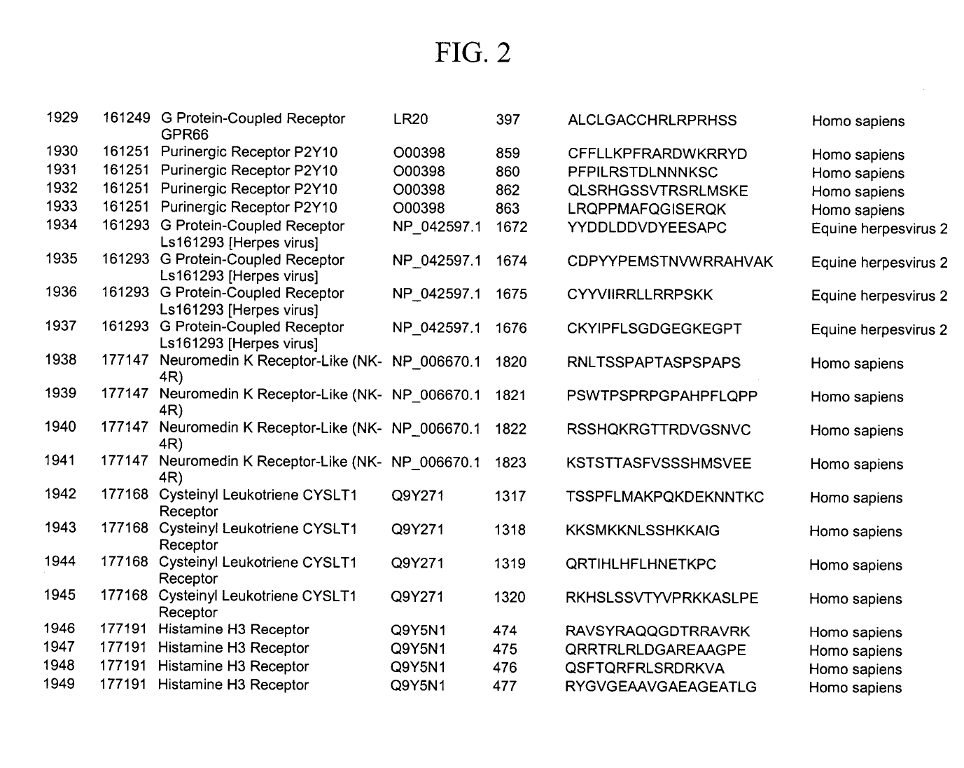

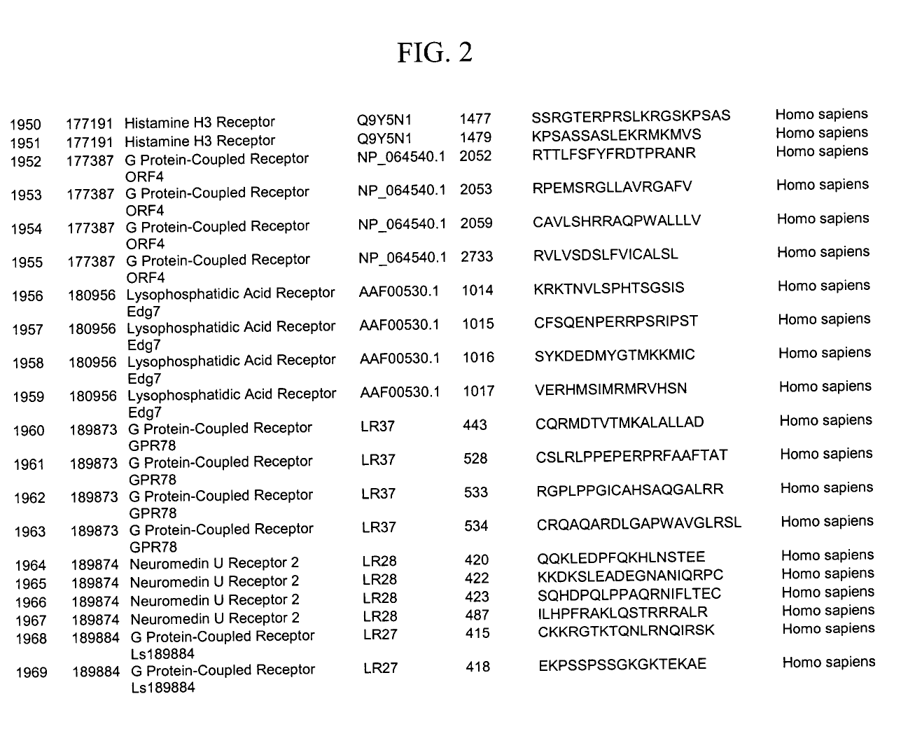

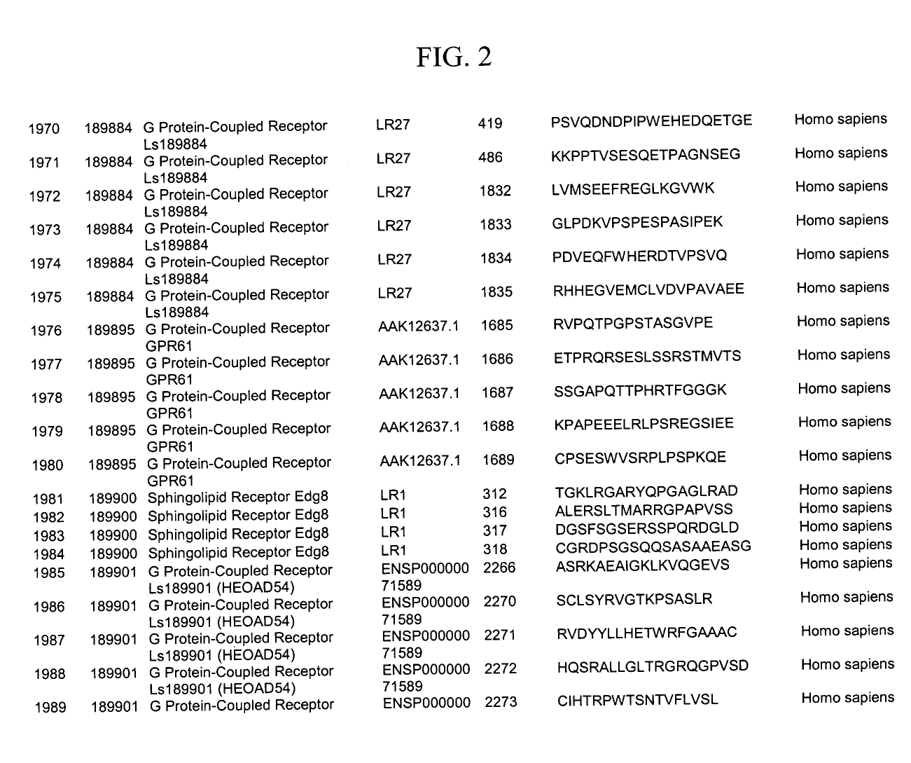

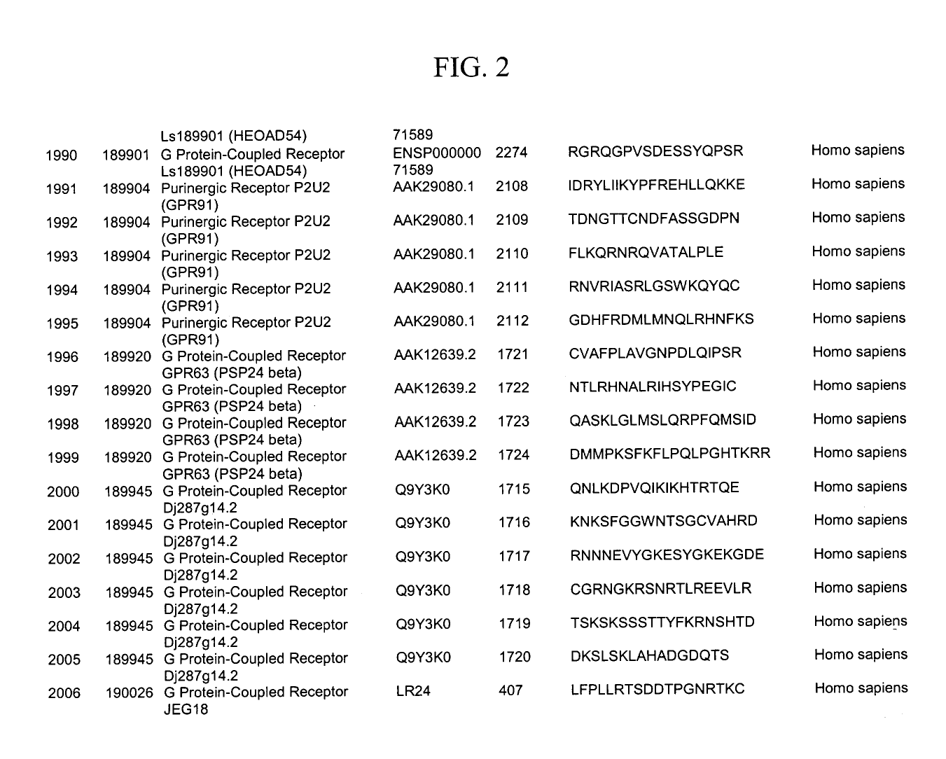

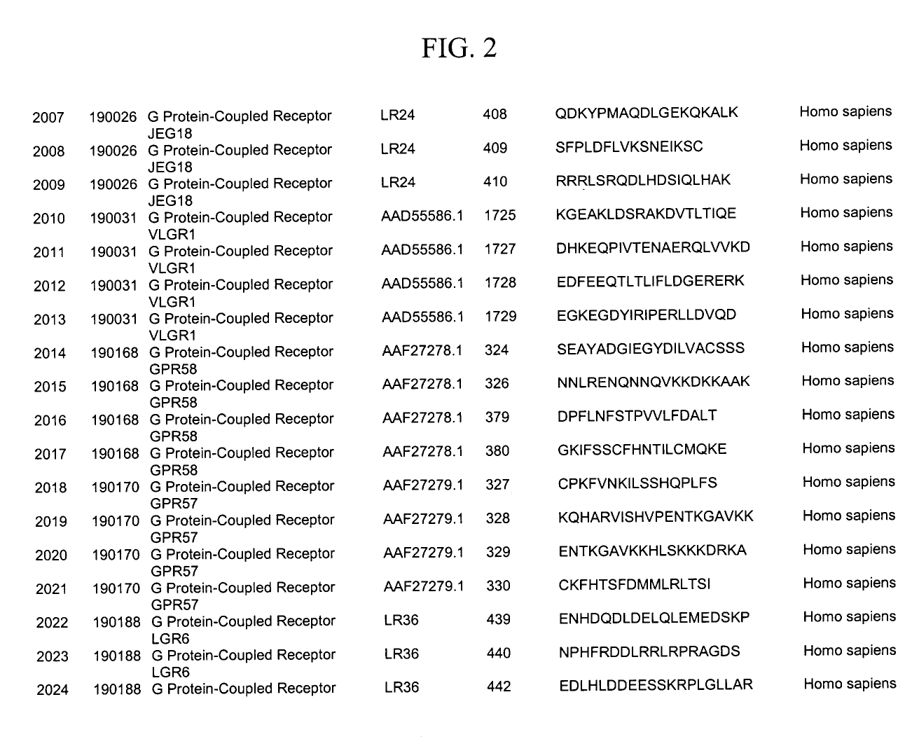

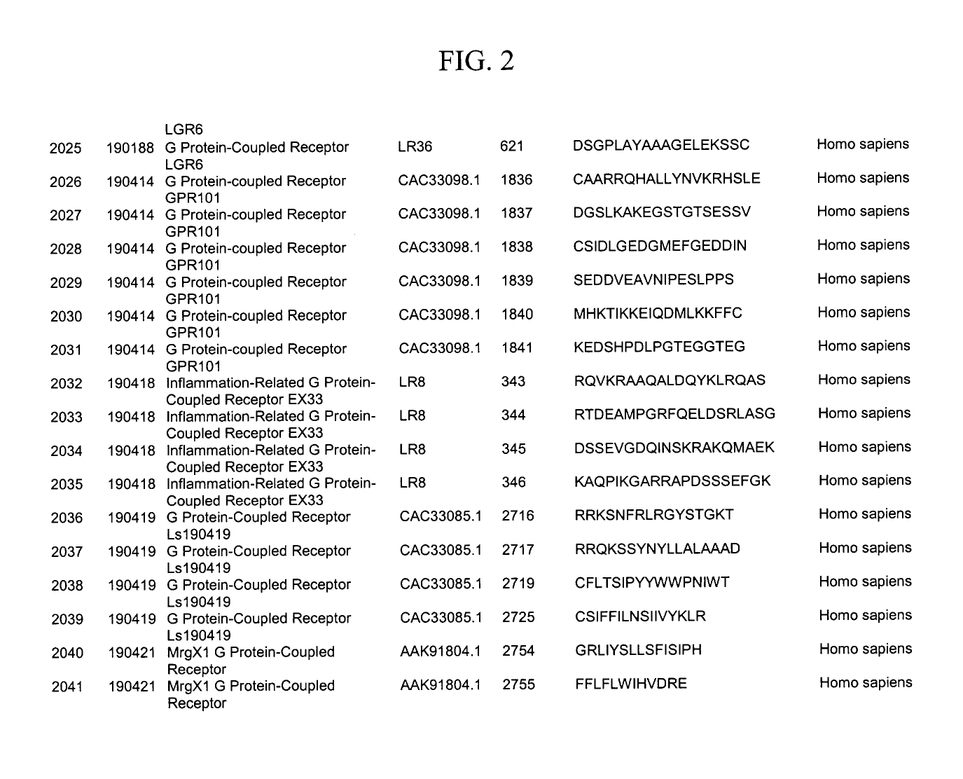

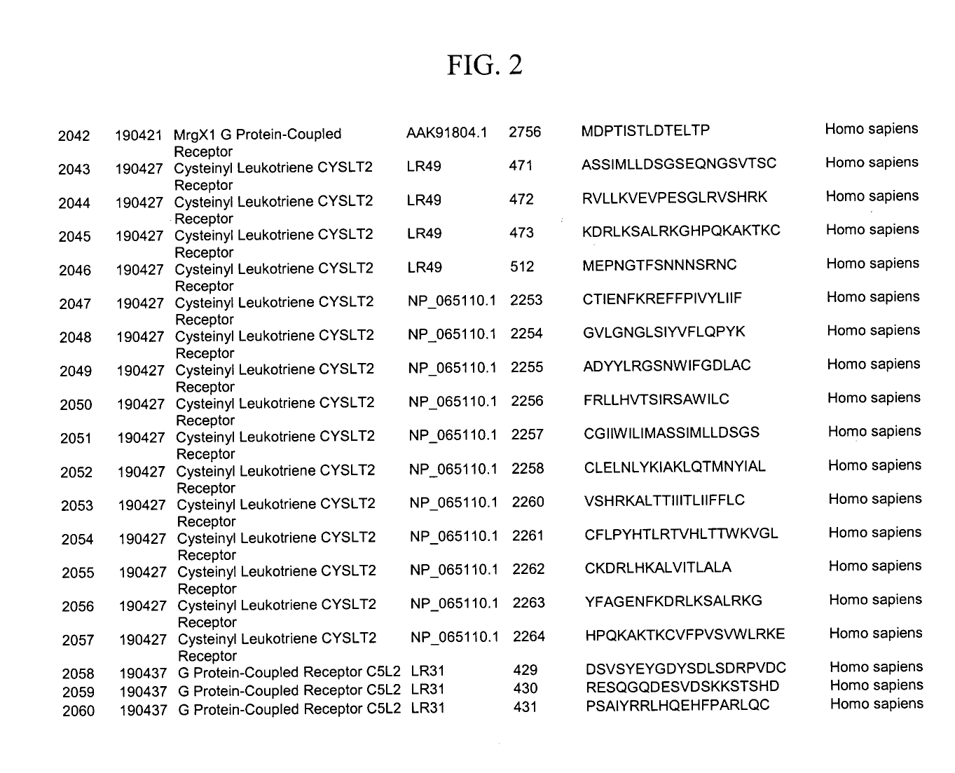

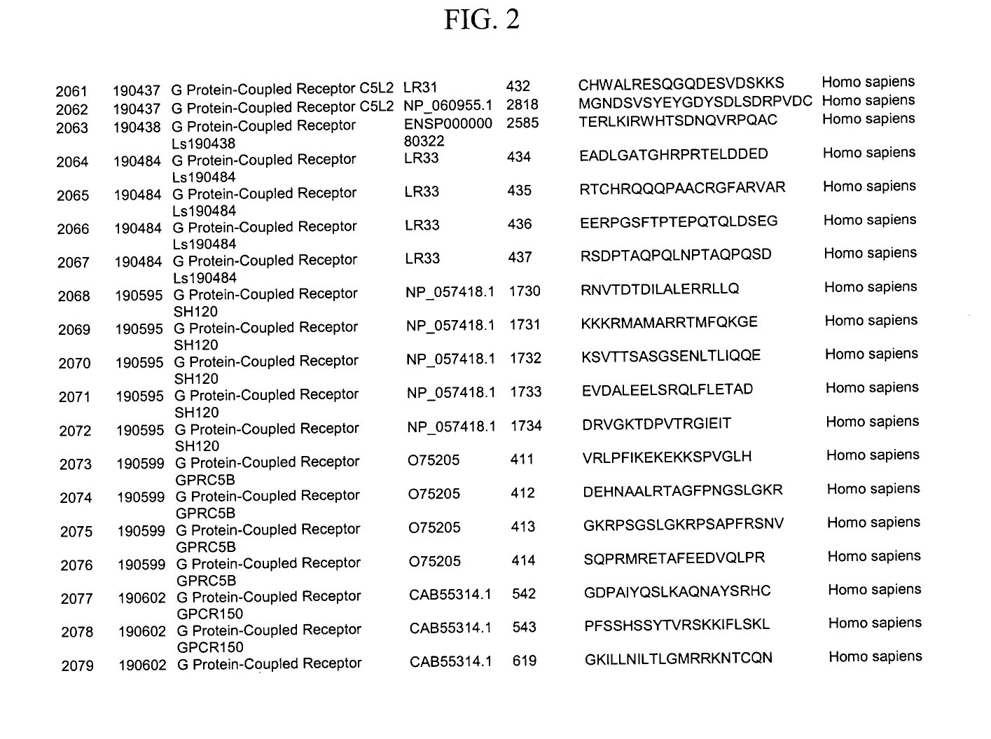

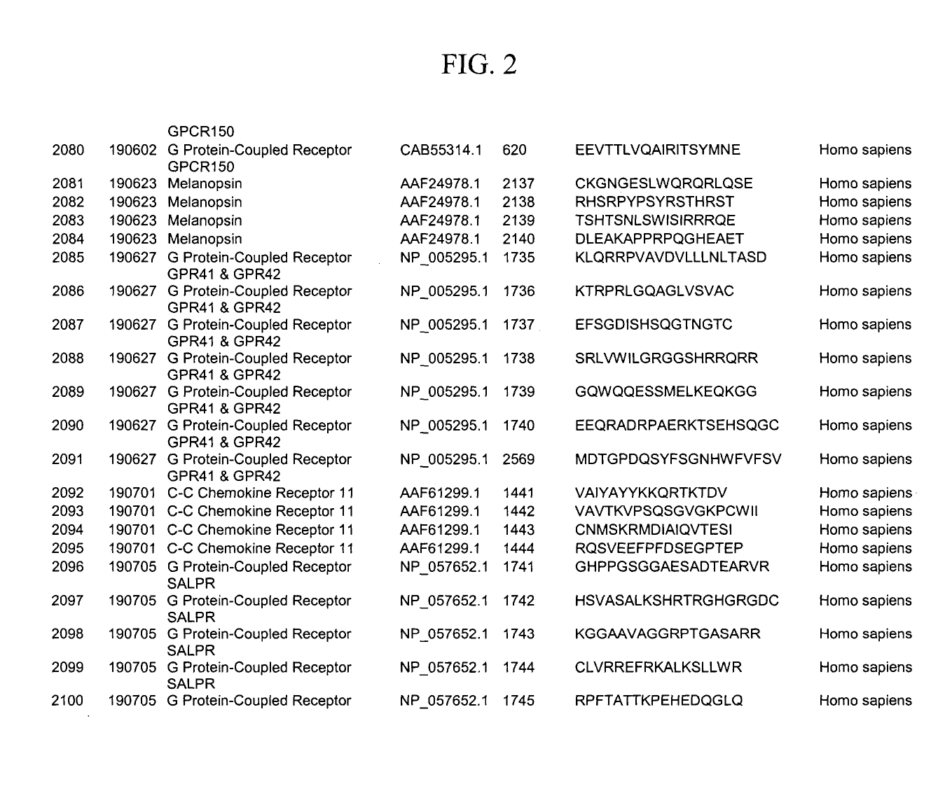

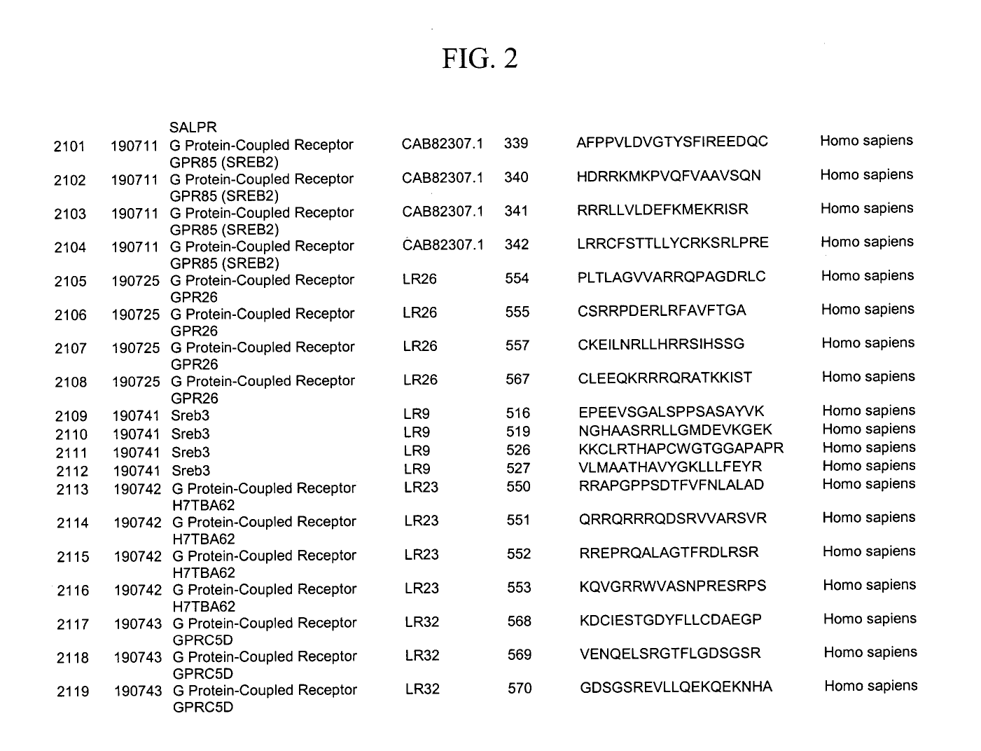

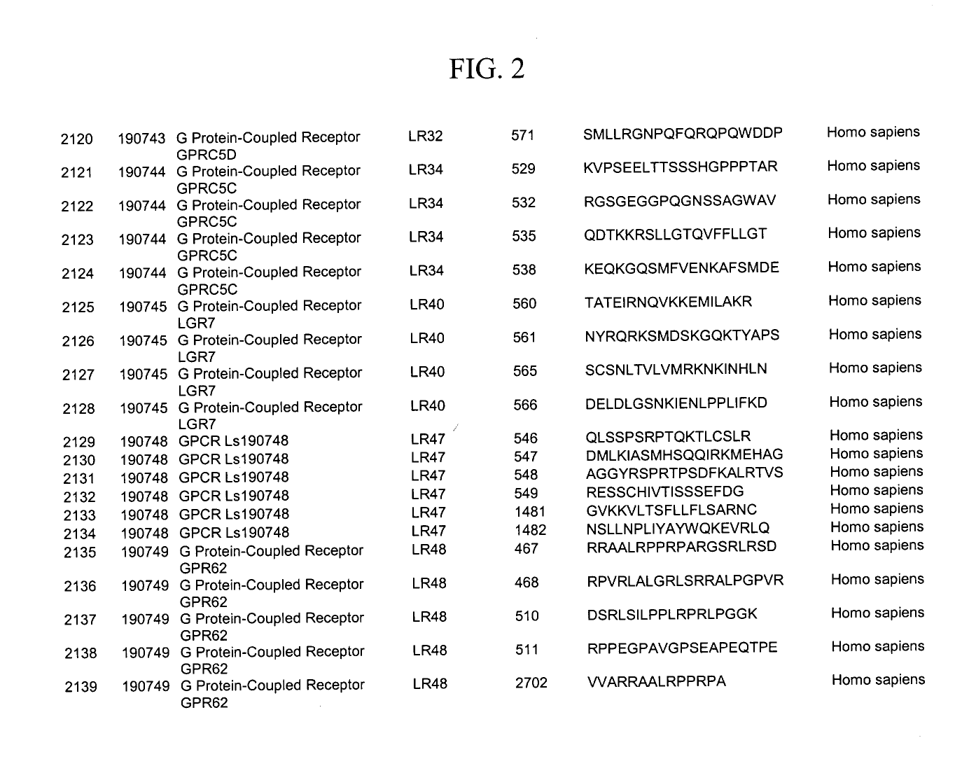

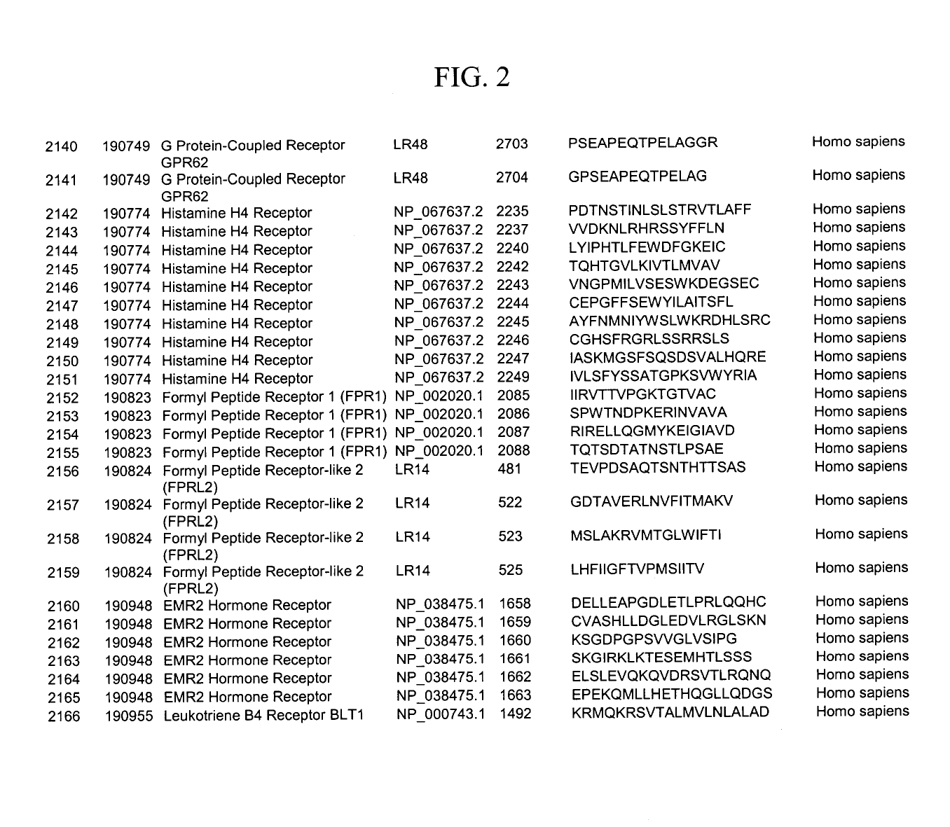

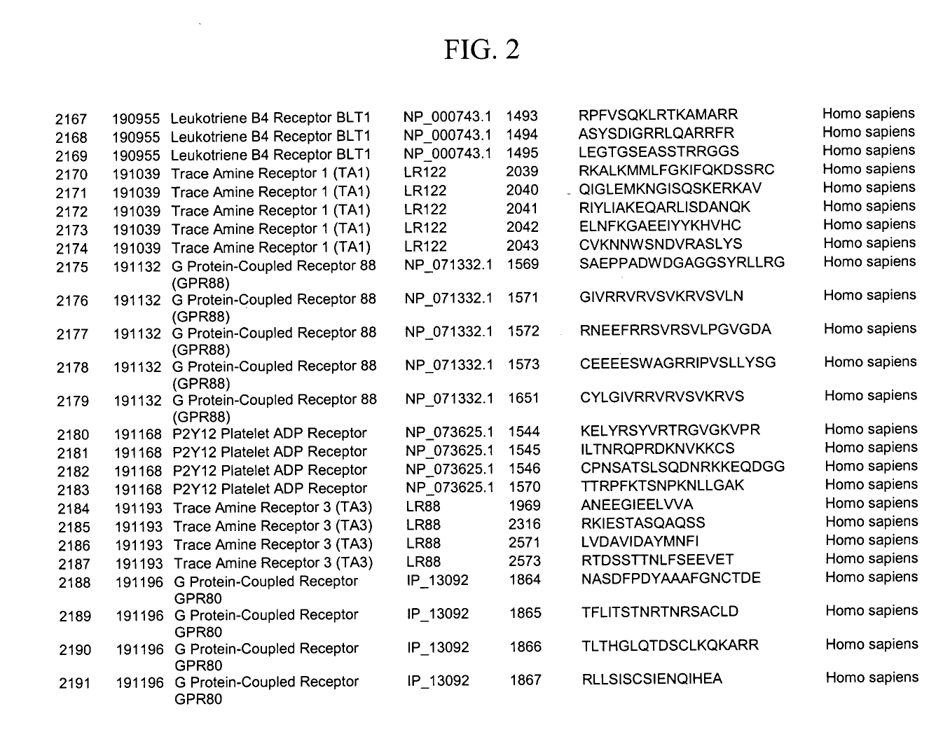

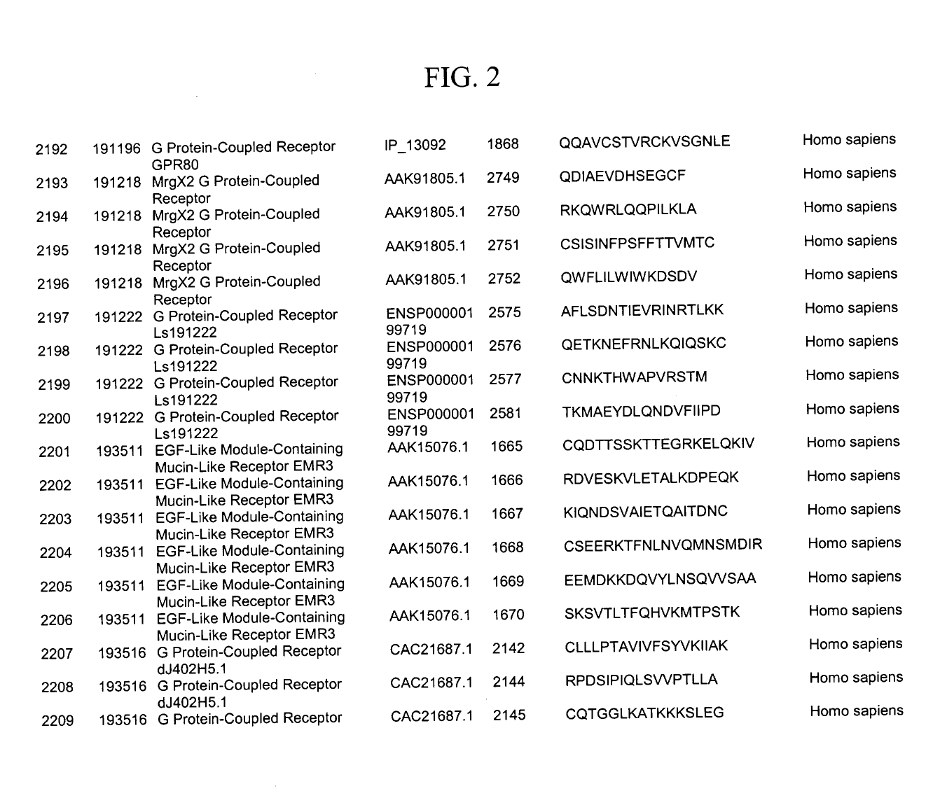

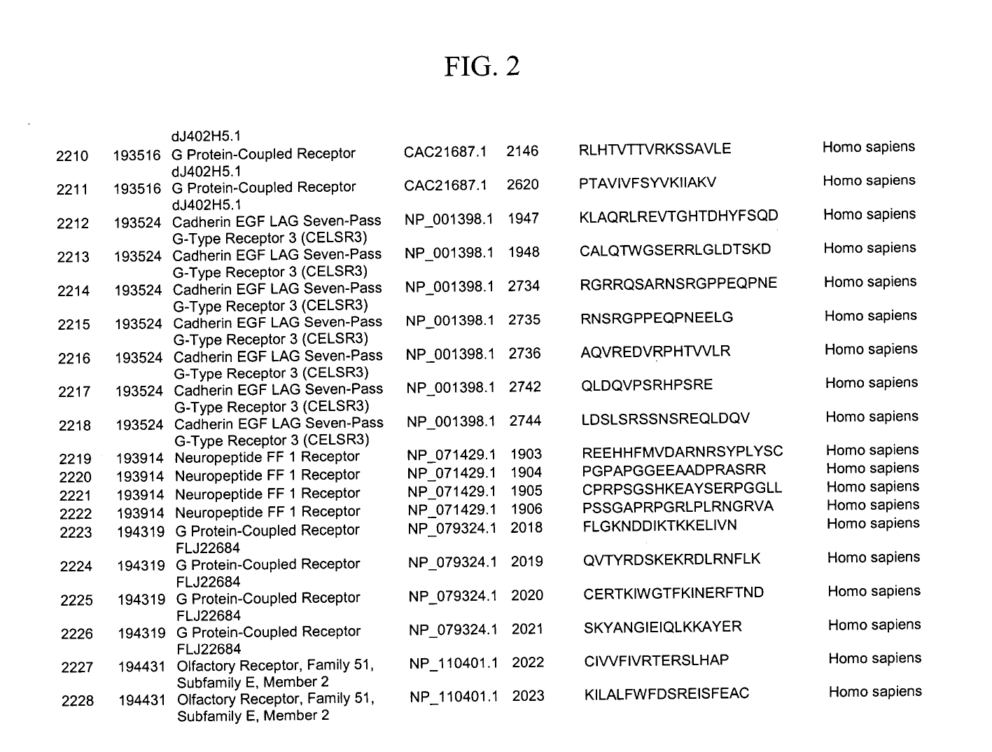

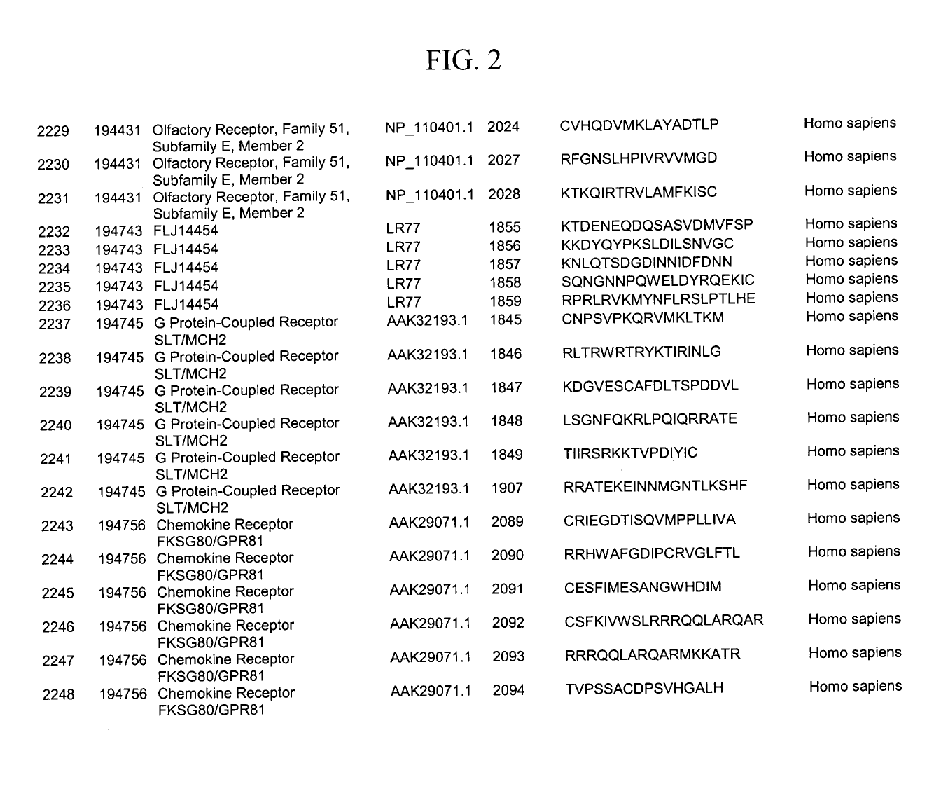

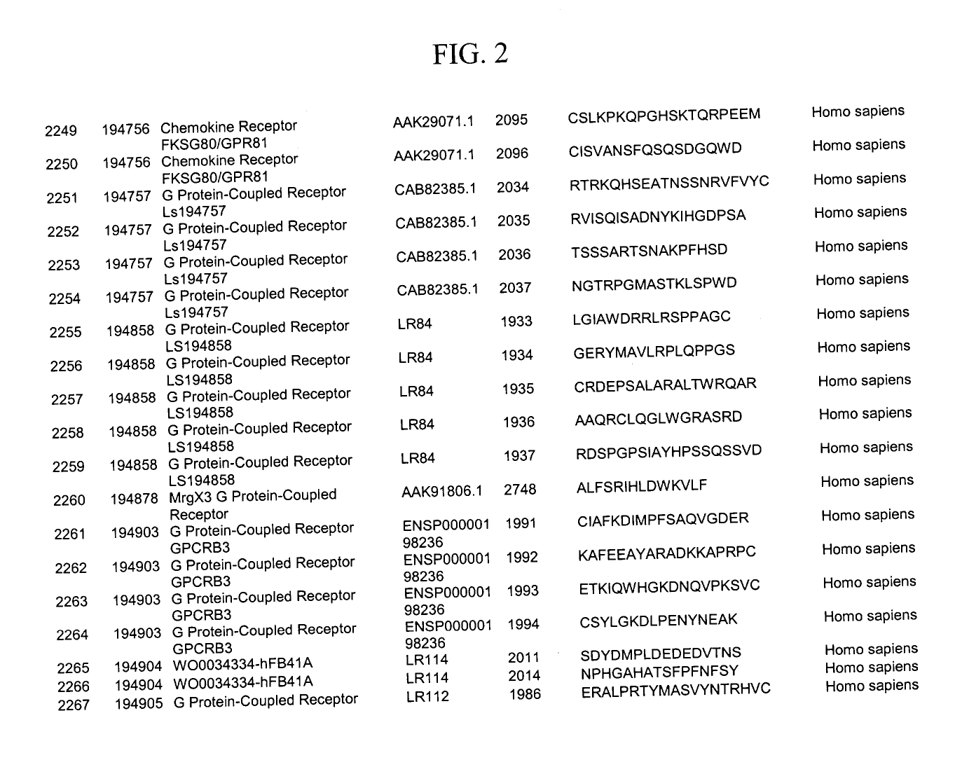

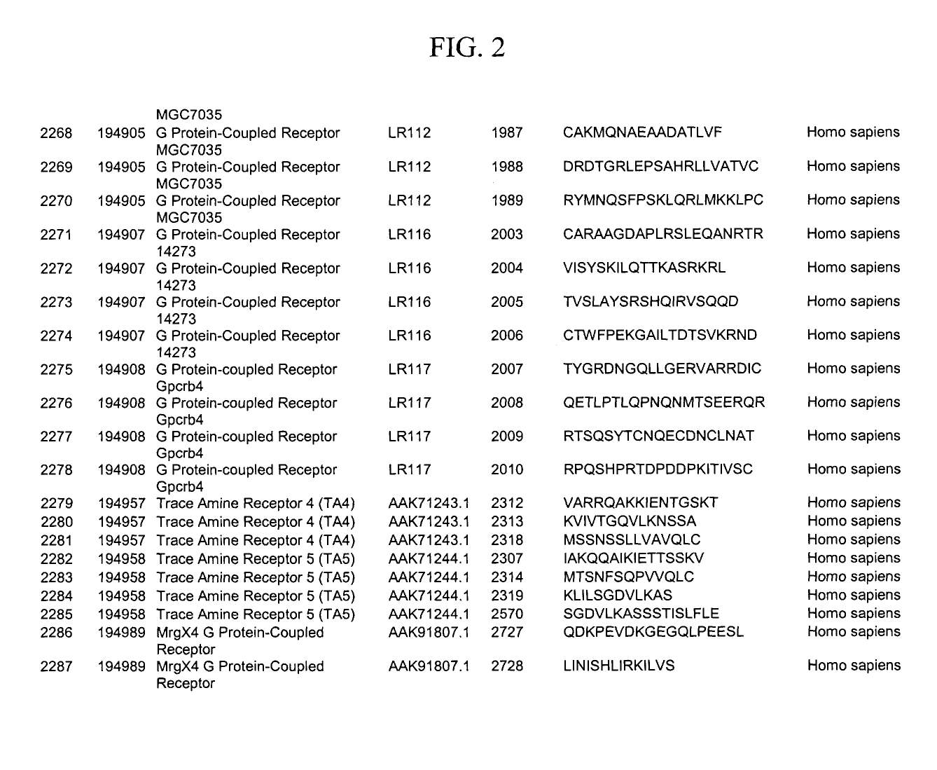

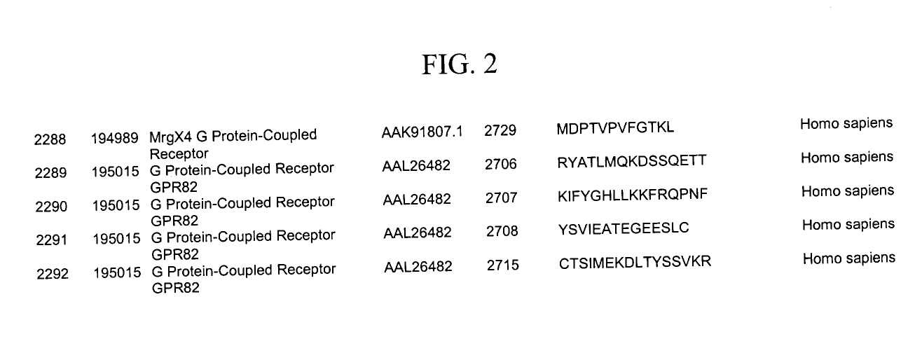

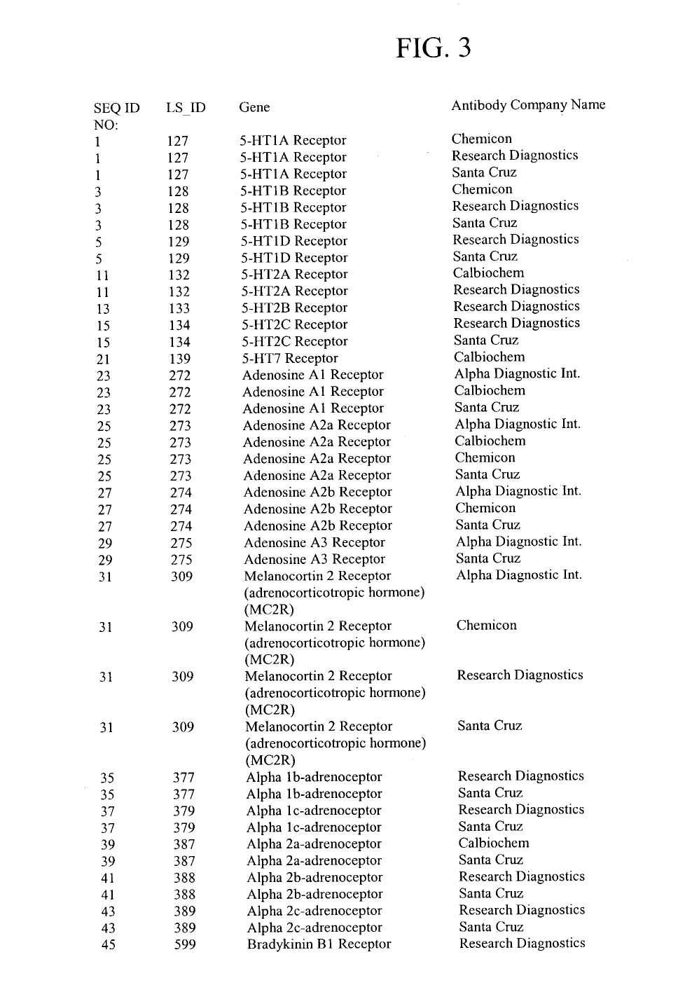

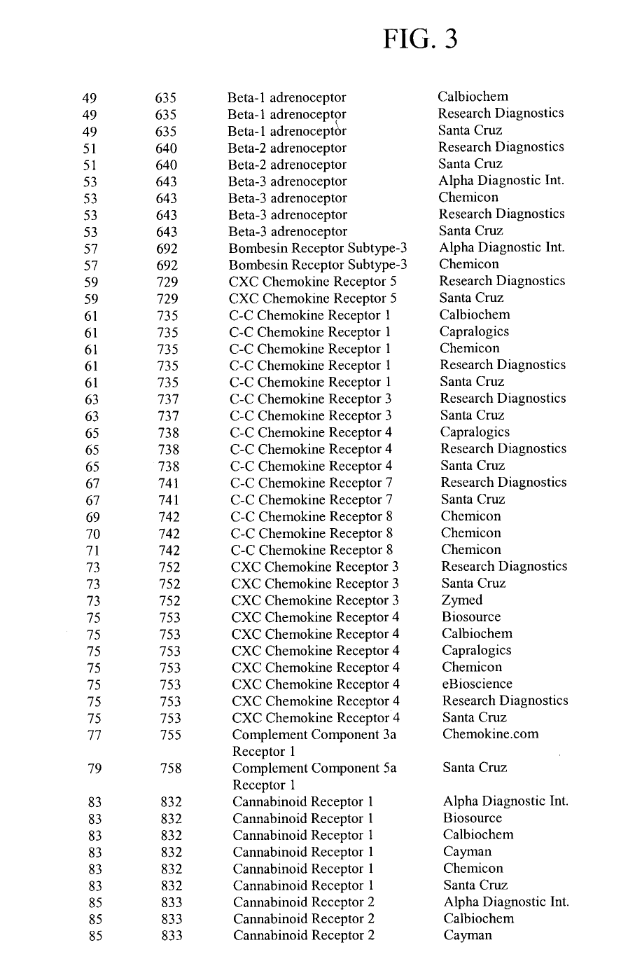

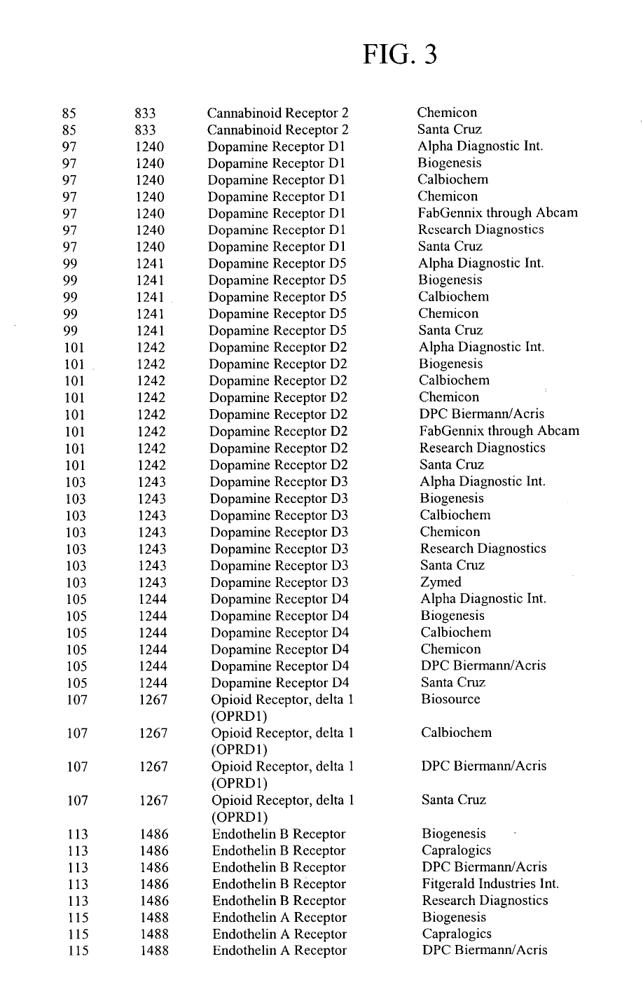

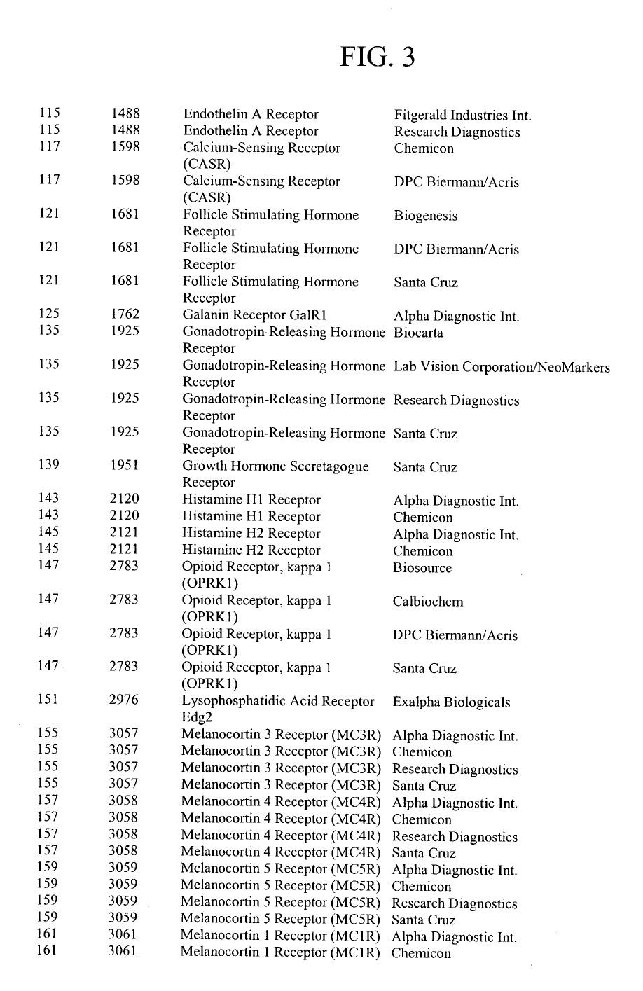

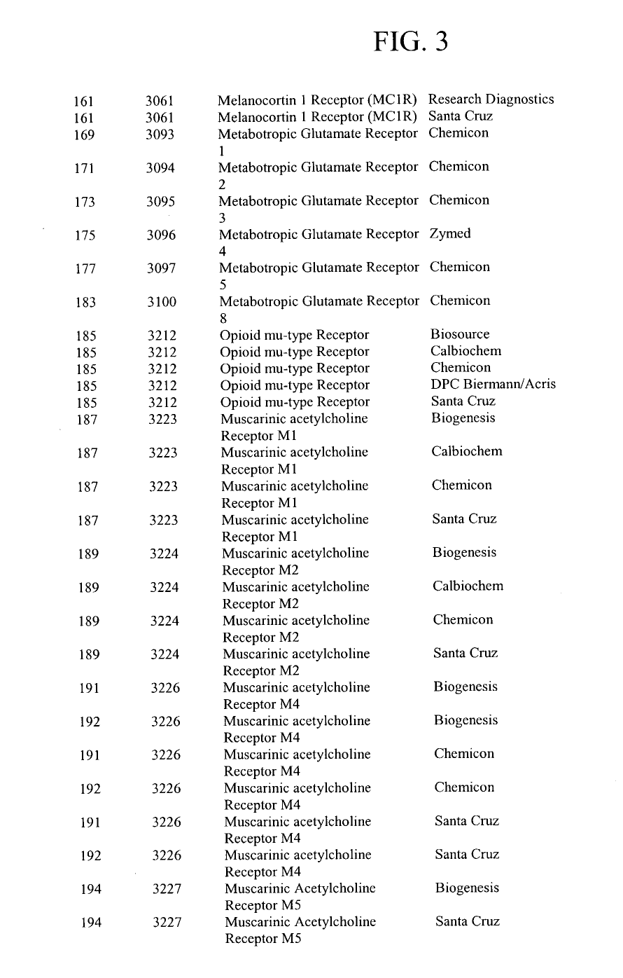

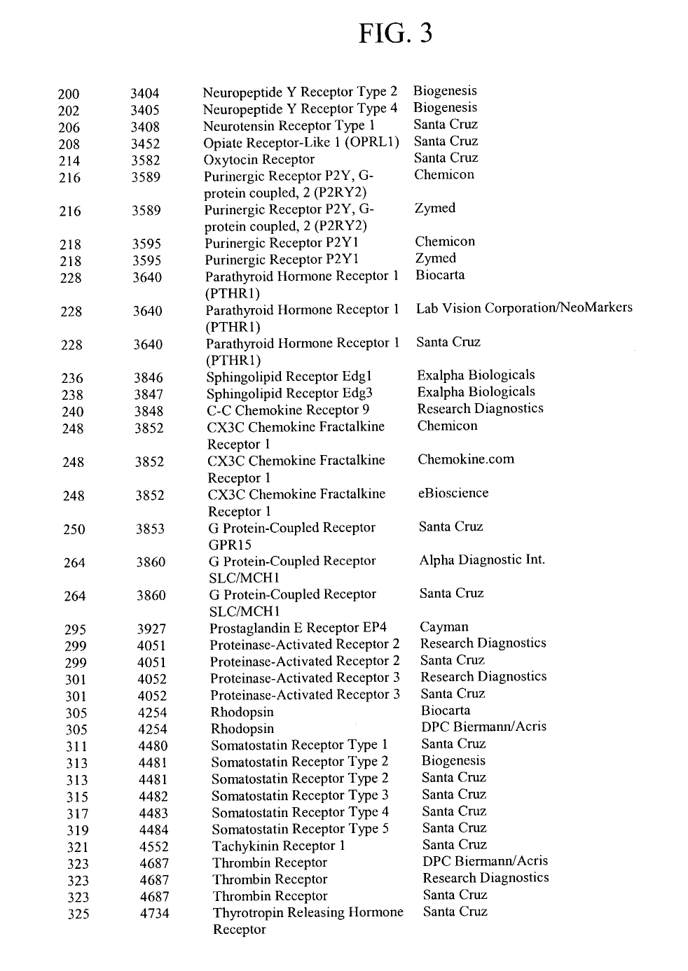

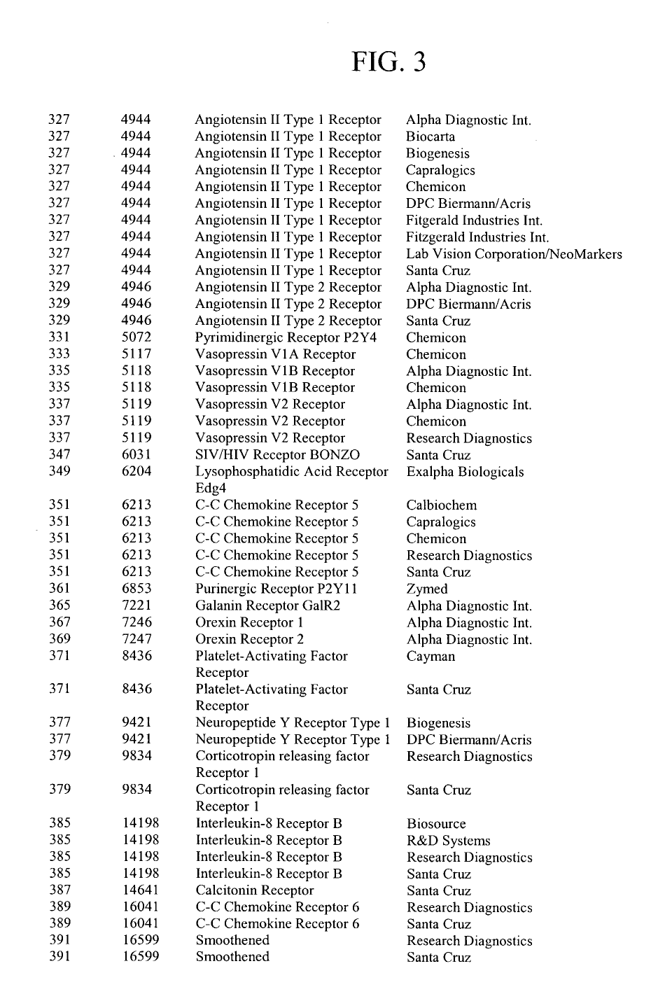

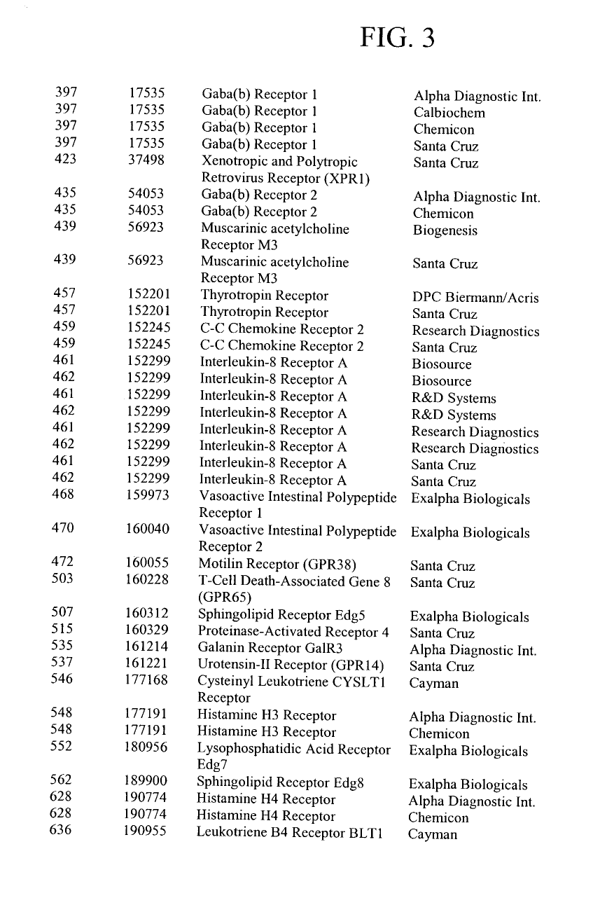

FIG. 1 sets forth the DNA and protein sequences for the GPCRs from which the antigenic peptides of the present invention were derived SEQ ID NOS. 1-691. FIG. 2 sets forth the amino acid sequences of exemplary antigenic peptides, SEQ ID NOS. 692-2292. The sequences in FIGS. 1 and 2 are listed according to SEQ ID NO and LSID, which is an identification number assigned to the given sequence in the LifeSpan Biosciences databases. The sequences in FIG. 2 also include an identifier LPID, which is also an identification number assigned to the given sequence in the LifeSpan Biosciences databases. FIG. 3 depicts GPCRs for which it has been reported that antibodies are commercially available, SEQ ID NOS. 1, 3, 5, 11, 13, 15, 21, 23, 25, 27, 29, 31, 35, 37, 39, 41, 43, 45, 49, 51, 53, 57, 59, 61, 63, 65, 67, 69, 70, 71, 73, 75, 77, 79, 83, 85, 97, 99, 101, 103, 105, 107, 113, 115, 117, 121, 125, 135, 139, 143, 145, 147, 151, 155, 157, 159, 161, 169, 171, 173, 175, 177, 183, 185, 187, 189, 191, 192, 194, 200, 202, 206, 208, 214, 216, 218, 228, 236, 238, 240, 248, 250, 264, 295, 299, 301, 305, 311, 313, 315, 317, 319, 321, 323, 325, 327, 329, 331, 333, 335, 337, 347, 349, 351, 361, 365, 367, 369, 371, 377, 379, 385, 387, 389, 391, 397, 423, 435, 439, 457, 459, 461, 462, 468, 470, 472, 503, 507, 515, 535, 537, 546, 548, 552, 562, 628, 636; Applicants do not represent that any of the antibodies in FIG. 3 that such antibodies are actually commercially available nor that they have any significant specificity nor affinity for the GPCRs reported. For GPCRs for which no antigens or antibodies were previously known, the present invention provides valuable antigenic peptides and antibodies (see, e.g., SEQ ID NOS. 704-712, 731-743, 774-777, 803-806, 821-824, 876-879, 890-916, 942-949, 965-970, 985-988, 994-1009, 1014-1020, 1025-1028, 1044-1048, 1053-1056, 1073-1086, 1114-1123, 1152-1160, 1173-1178, 1188-1197, 1210-1227, 1232-1244, 1258-1270, 1280-1303, 1309-1368, 1373-1377, 1386-1389, 1394-1402, 1462-1482, 1496-1525, 1542-1549, 1557-1563, 1583-1649, 1656-1679, 1684-1688, 1693-1732, 1744-1752, 1765-1839, 1846-1854, 1855-1866, 1871-1917, 1926-1941, 1952-1955, 1960-1980, 1985-2141, 2152-2165, and 2170-2292.); for GPCRs for which antigens or antibodies are known, the present invention provides improved antigens in the form of antigenic peptides and improved antibodies (see, e.g., SEQ ID NOS. 692-703, 713-730, 744-802, 807-820, 825-875, 880-889, 917-941, 950-964, 971-984, 989-993, 1010-1013, 1021-1024, 1029-1043, 1049-1052, 1057-1072, 1087-1113, 1124-1151, 1161-1172, 1179-1187, 1198-1209, 1228-1231, 1245-1257, 1271-1279, 1304-1308, 1369-1372, which are antigenic peptides derived from GPCRs for which antibodies are reportedly commercially available). The antigenic peptides and antibodies, and uses and assays, etc., related to the antigenic peptides, are discussed further below.

The discussion herein, including the following passages, has been separated by headings for convenience. The disclosure under a given heading is not restricted to that heading. For example, the discussion in the definitions section is a part of the disclosure of the invention, the discussion on antigenic peptides also contains discussion related to probes and diagnostics, and the discussion on antibodies contains discussion related to therapeutic compositions, etc.

B. Definitions

The following paragraphs provide a non-exhaustive list of definitions of some of the terms and phrases as used herein. All terms used herein, including those specifically described below in this section, are used in accordance with their ordinary meanings unless the context or definition indicates otherwise. Also unless indicated otherwise, except within the claims, the use of “or” includes “and” and vice-versa. Non-limiting terms are not to be construed as limiting unless expressly stated (for example, “including” means “including without limitation” unless expressly stated otherwise).

The terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

“Agonist” indicates a substance, such as a molecule or compound, that interacts with a particular GPCR, for example by binding to the GPCR, to activate, increase, or prolong the amount or the duration of the effect of the biological activity or functionality of the GPCR. Agonists include proteins, nucleic acids, carbohydrates, or any other molecules that bind to and positively modulate the effect of the GPCR. Agonists and other modulators of the particular GPCR can be identified using in vitro or in vivo assays for G protein-coupled receptor expression or G protein-mediated signaling. For example, assays for agonists and other modulators include expressing a particular GPCR in cells or cell membranes, applying putative modulator compounds in the presence or absence of a specific known or putative ligand and then determining the functional effects on the particular GPCR-mediated signaling. Samples or assays comprising a particular GPCR that are treated with a potential agonist or other modulator are compared to control samples without the agonist or other modulator to examine the extent of modulation. Control samples can be assigned a relative activity value for the particular GPCR of 100%. Agonist activity on a particular GPCR is achieved when the G protein-coupled receptor activity value relative to the control is at least about 110%, optionally about 150%, preferably about 200-500%, or about 1000-3000% or higher. Down-modulation (for example by an antagonist) of a particular GPCR is achieved when the particular GPCR activity value relative to the control is at most about 90%, typically about 80%, optionally about 50% or about 25-0% of the 100% value.

“Aggregate,” see Complex.

“Algorithm” refers to a detailed sequence of actions to perform to accomplish some task. In computer programming, refers to instructions given to the computer.

“Allele” or “allelic sequence” indicates an alternative form of the gene encoding the GPCR. Alleles may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to alleles are generally ascribed to natural deletions, additions, or substitutions of nucleotides. Each of these types of changes may occur alone or in combination with the others, one or more times in a given sequence.

“Altered” nucleic acid sequences encoding the GPCR include those sequences with deletions, insertions, or substitutions of different nucleotides, resulting in a polynucleotide encoding the same GPCR or a polypeptide variant with at least one substantial structural or functional characteristic of the GPCR. Included within this definition are polymorphisms that may or may not be readily detectable using a particular oligonucleotide probe against the polynucleotide encoding the GPCR. “Altered” proteins may contain deletions, insertions, or substitutions of amino acid residues that produce a silent change and result in a functionally equivalent GPCR. Deliberate amino acid substitutions may be made on the basis of similarity in polarity, charge, solubility, hydrophobicity, hydrophilicity, or the amphipathic nature of the residues, as long as the biological or immunological activity of the GPCR is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine.

“Alternative splicing” refers to different ways of cutting and assembling exons to produce mature mRNAs.

“Amino acid” refers generally to any of a class of organic compounds that contains at least one amino group, —NH₂, and one carboxyl group, —COOH. The alpha-amino acids, RCH(NH₂)COOH, are the building blocks from which proteins are typically constructed. Amino acid can also refer to artificial chemical analogues or mimetics of a given amino acid as described, depending on the context.

“Amino acid sequence” refers to a string of amino acids, such as an oligopeptide, peptide, polypeptide, or protein sequence, or a fragment of any of these, including naturally occurring or synthetic molecules and those comprising an artificial chemical analogue or mimetic of a given amino acid. In this context, “biologically active fragments,” “biologically functional fragments,” “immunogenic fragments,” and “antigenic fragments” refer to fragments of the GPCR that are preferably about 15, 25, or 50 or more amino acids in length and that retain a substantial amount of such activity of the GPCR. Where “amino acid sequence” refers to an amino acid sequence of a naturally occurring protein molecule, “amino acid sequence” and like terms are not necessarily limited to the complete native amino acid sequence associated with the recited protein molecule.

“Amplification” indicates the production of additional copies of something, such as a nucleic acid sequence. Amplification can be generally carried out using polymerase chain reaction (PCR) technologies or other technologies such as the cycling probe reaction (CPR) that are well known in the art. See, e.g., Dieffenbach, C. W. and G. S. Dveksler, PCR Primer, a Laboratory Manual, pp.1-5, Cold Spring Harbor Press, Plainview, N.Y. (1995); U.S. Pat. Nos. 5,660,988, 5,731,146 and 6,136,533.

“Amplification primers” are oligonucleotides such as natural, analog or artificially created nucleotides that can serve as the basis for the amplification of a selected nucleic acid sequence. They include, for example, both PCR primers and ligase chain reaction oligonucleotides.

“Analog” or “variant” indicates a GPCR or antigenic peptide that has been modified by deletion, addition, modification, or substitution of one or more amino acid residues compared to the wild-type sequence. Analogs encompass allelic and polymorphic variants, and also muteins and fusion proteins that comprise all or a significant part of such GPCR, e.g., covalently linked via side-chain group or terminal residue to a different protein, polypeptide, or moiety (fusion partner). Variants of a particular GPCR protein refer to an amino acid sequence that is altered by one or more amino acids, for example by one or more amino acid substitution, insertion, deletion or modification, or proteins with or without associated native-pattern glycosylation. The variant may have “conservative” changes. Such “conservative” changes generally are well known in the art and readily determinable for a particular GPCR in view of the present application. Conservative changes include, for example, substitutions where a substituted amino acid has similar structural or chemical properties to the amino acid it replaced (e.g., negatively charged amino acids include aspartic acid and glutamic acid; positively charged amino acids include lysine, arginine, histidine, asparagine, and glutamine; amino acids containing sulfur include methionine and cysteine; polar hydroxy amino acids include serine, threonine, and tyrosine; large hydrophobic amino acids include phenylalanine and tryptophan; small hydrophobic amino acids include alanine, leucine, isoleucine, and valine). A variant may also have “nonconservative” changes which means that the replacement amino acid provides some substantial change in the amino sequence.

A variant preferably retains at least about 90% identity, and more preferably at least about 95% identity. Within certain embodiments, such variants contain alterations such that the ability of the variant to induce an immunogenic response is not substantially eliminated; in some embodiments the ability to an immunogenic response is not substantially diminished. Modifications of amino acid residues may include but are not limited to aliphatic esters or amides of the carboxyl terminus or of residues containing carboxyl side chains, O-acyl derivatives of hydroxyl group-containing residues, and N-acyl derivatives of the amino-terminal amino acid or amino-group containing residues, e.g., lysine or arginine. Guidance in determining which and how many amino acid residues may be substituted, inserted, deleted or modified without diminishing immunological or biological activity may be found in view of the present application using any of a variety of methods and computer programs known in the art, for example, DNASTAR software. Properties of a variant may generally be evaluated by assaying the reactivity of the variant with, for example, antibodies as described herein or evaluating a biological activity characteristic of the native protein as described herein or as known in the art in view of the present application. Certain polynucleotide variants are capable of hybridizing under appropriately stringent conditions to a naturally occurring DNA sequence encoding a particular GPCR protein (or a complementary sequence). Such hybridizing nucleic acid sequences are also within the scope of this invention.

“Antagonist” refers to a molecule which interacts with a particular GPCR, for example by binding to the particular GPCR, and prevents, inactivates, decreases or shortens the amount or the duration of the effect of the biological activity of the GPCR. Antagonists include proteins, nucleic acids, carbohydrates, antibodies, or any other molecules that so affect the GPCR. Antagonists can be identified, for example, using appropriate screens corresponding to those described for agonists above and elsewhere herein or as would be apparent to those skilled in the art in view of the present application.

“Antibody” indicates one type of binding partner, typically encoded by an immunoglobulin gene or immunoglobulin genes, and refers to, for example, intact monoclonal antibodies (including agonist and antagonist antibodies), polyclonal antibodies, phage display antibodies, and multispecific antibodies (e.g., bispecific antibodies) formed, for example, from at least two intact antibodies. Antibody also refers to fragments thereof, which comprise a portion of an intact antibody, generally the antigen-binding or variable region of the intact antibody that are capable of binding the epitopic determinant. Examples of antibody fragments include Fab, Fab′, F(ab′)₂, and Fv fragments, diabodies, linear antibodies, single-chain antibody molecules, and multispecific antibodies formed from antibody fragments. See U.S. Pat. No. 6,214,984. Antibody fragments may be synthesized by digestion of an intact antibody or synthesized de novo either chemically or utilizing recombinant DNA technology. Antibodies according to the present invention have at least one of adequate specificity, affinity and capacity to perform the activities desired for the antibodies. Antibodies can, for example, be monoclonal, polyclonal, or combinatorial. Antibodies that bind GPCR polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

“Antigenic determinant” refers to the antigen recognition site on an antigen (i.e., epitope). Such antigenic determinant may also be immunogenic.

“Antisense” refers to any composition containing a nucleic acid sequence that is complementary to a specific nucleic acid sequence. “Antisense strand” refers to a nucleic acid strand that is complementary to the “sense” strand. Antisense molecules may be produced by any method including transcription or synthesis including synthesis by ligating the gene(s) of interest in a reverse orientation to a desired promoter that permits the synthesis of a complementary strand. Once introduced into a cell, the complementary nucleotides can combine with natural sequences produced by the cell to form duplexes and to block either transcription or translation. The designation “negative” can refer to the antisense strand, and the designation “positive” can refer to the sense strand.

“Biologically active” or “biologically functional,” when referring to an antigenic peptide, indicates that the antigenic peptide induces an immunogenic response specific for the antigenic peptide and thus for the GPCR from which is was obtained. A variant, fragment, etc., of an antigenic peptide is “biologically active” or “biologically functional” if the ability to induce the specific immunogenic response is not substantially diminished. The term “not substantially diminished” means retaining a functionality that is at least about 90% of the functionality of the native antigenic peptide. Appropriate assays designed to evaluate such functionality may be designed based on existing assays known in the art in view of the present application, or on the representative assays provided herein.

“Annotation” refers to the provision of helpful or identifying information about a GPCR or other open reading frame (ORF), such as locus name, key words, and Medline references.

“BLAST” refers to the Basic Local Alignment Search Tool, which is a technique for detecting ungapped sub-sequences that match a given query sequence. BLAST can be used as a preliminary step for detecting ORF boundaries.

“BLASTP” refers to a BLAST program that compares an amino acid query sequence against a protein sequence database.

“BLASTX” refers to a BLAST program that compares the six-frame conceptual translation products of a nucleotide query sequence (both strands) against a protein sequence database. BLASTX can be used to create a sub-database of ORFs which may exist on a contig, and to identify the best match between one of these ORFs and a sequence in an external database.

“Buffer” refers to a component in a solution to provide a buffered solution that resists changes in pH by the action of its acid-base conjugate components.

“CDS” refers to the GenBank DNA sequence entry for coding sequence. A coding sequence is a sub-sequence of a DNA sequence that is surmised to encode a gene. A complete gene coding sequence begins with an “ATG” and ends with a stop codon.

“Clone” in molecular biology refers to a vector carrying an insert DNA sequence.

“Cloning” in molecular biology refers to a recombinant DNA technique used to produce multiple, up to millions or more, copies of a DNA sequence. The DNA sequence is inserted into a small carrier or vector (e.g., plasmid, bacteriophage, or virus) and inserted into a host cell for amplification or expression.

“Cluster” refers to a group of ORFs related to one another by sequence homology. Clusters are generally determined by a specified degree of homology and overlap (e.g., a stringency).

“Comparison window” indicates a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are aligned to enhance sequence similarity. Methods of alignment of sequences for comparison will be readily apparent to a person of ordinary skill in the art in view of the present application.

“Complementary” or “complementarity” refers to the natural binding of polynucleotides by base pairing. For example, the sequence “A-G-T” binds to the complementary sequence “T-C-A.” Complementarity between two single-stranded molecules may be “partial,” such that only some of the nucleic acids bind, or it may be “complete,” such that all of the nucleotides of at least one of the single-stranded molecules binds to corresponding nucleotides of the other single-stranded molecule. The degree of complementarity between nucleic acid strands has significant effects on the efficiency and strength of the hybridization between the nucleic acid strands. This can be of particular importance in amplification reactions, which can depend upon binding between nucleic acids strands, and in the design and use of peptide nucleic acid (PNA) molecules.

“Complex,” or “aggregate,” indicates a dimer or multimer formed between at least two proteins or other macromolecules, for example a GPCR and its ligand.

“Composition” indicates a combination of multiple substances into a mixture.

“Composition comprising a given amino acid sequence” refers broadly to any composition containing the given amino acid sequence. The composition may comprise a dry formulation, an aqueous solution, or a sterile composition.

“Consensus sequence” refers to the sequence that reflects the most common choice of base or amino acid at each position from a series of related DNA, RNA, or protein sequences. Areas of particularly good agreement often represent conserved functional domains. The generation of consensus sequences has typically been subjected to intensive mathematical analysis.

“Conservative changes” to an amino acid sequence, see Analog.

“Deletion” refers to a change in the amino acid or nucleotide sequence that results in the absence of one or more amino acid residues or nucleotides.

“Derivative” refers to chemical modification of an antigenic peptide, or of an antibody specific for and created from the antigenic peptide. A derivative peptide can be modified, for example, by glycosylation or pegylation.

“Diabodies” refers to one type of antibody comprising small antibody fragments with two antigen-binding sites, which fragments comprise a heavy-chain variable domain (VH) connected to a light-chain variable domain (VL) on the same polypeptide chain (VH-VL). By using a linker that is too short to allow pairing between the two domains on the same chain, the domains pair with the complementary domains of another chain and create two antigen-binding sites. Diabodies are described, for example, in EP 404,097; WO 93/11161; and Holliger et al., Proc. Natl. Acad. Sci. USA, 90:6444-6448 (1993).

“Database” refers to a structured format for organizing and maintaining information or data, a collection of data records, in a computer-readable form that can be rapidly and easily retrieved. A database is typically stored in a computer-readable memory. Records may comprise web pages, graphics, audio files, text files, or links. Records may or may not be further broken into fields. Database records are usually indexed and come with a search interface to find records of interest.

“E-value” refers to a result of a FASTA analysis. The number indicates the probability that a match between two sequences is due to random chance.

“Expression vector” is a specialized vector constructed so that the gene inserted in the vector can be expressed in the cytoplasm of a host cell.

“FASTA” refers to a modular set of sequence comparison programs used to compare an amino acid or DNA sequence against all entries in a sequence database. FASTA was written by Professor William Pearson of the University of Virginia Department of Biochemistry. The program uses the rapid sequence algorithm described by Lipman and Pearson (1988) and the Smith-Waterman sequence alignment protocol. FASTA performs a protein to protein comparison.

“FASTX” refers to a module of the FASTA protocol used to define optimal ORF boundaries while searching for genes. FASTX uses a nucleotide to protein sequence comparison.

“Fragment,” see Portion.

“GenBank” refers to a family of public databases comprising nucleic acid and amino acid sequence information, including the GenPept bacterial peptide database.

“Gene” refers to the basic unit of heredity that carries the genetic information for a given RNA or protein molecule. A gene is composed of a contiguous stretch of DNA and contains a coding region that is flanked on each end by regions that are transcribed but not translated. A gene is a segment of DNA involved in producing a biologically active or biologically functional polypeptide chain.

“Heterologous” indicates a nucleic acid that comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“Hit Threshold” refers to a pre-set E-value or P-value for evaluating sequence matches. For example, this value can be set at le-6 for finding genes; and at le-15 for clustering genes.

“Homology” refers to a degree of complementarity. There may be partial homology or complete homology. The word “identity” may substitute for the word “homology.” A partially complementary sequence that at least partially, and substantially, inhibits a corresponding sequence from hybridizing to a target nucleic acid is referred to as “substantially homologous.”The inhibition of hybridization of the completely complementary sequence to the target sequence may be examined using a hybridization assay (e.g., Southern or Northern blot, in situ hybridization, solution hybridization) under conditions of reduced stringency. A substantially homologous sequence or hybridization probe will compete for and inhibit the binding of a completely homologous sequence to the target sequence under stringency conditions that inhibit non-specific binding but permit specific binding. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second, non-complementary target sequence.

“Humanized antibody” refers to antibody molecules in which the amino acid sequence in the non-antigen-binding regions has been altered so that the antibody more closely resembles a human antibody, and still retains its original binding ability. Typically, humanized antibodies are human immunoglobulins (recipient antibody) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework residues of the human immunoglobulin are replaced by corresponding non-human residues. Furthermore, humanized antibodies may comprise residues that are found neither in the recipient antibody nor in the imported CDR or framework sequences. These modifications are typically made to further refine and optimize antibody performance. In general, the humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin sequence. The humanized antibody optimally also will comprise at least a portion of an immunoglobulin constant region (Fc), typically that of a human immunoglobulin. For further details see, e.g., Jones et al., Nature, 321:522-525 (1986); Reichmann et al., Nature, 332:323-329 (1988); and, Presta, Curr. Op. Struct. Biol., 2:593-596 (1992).

“Identity,” see Homology.

“Immunocytochemistry” refers to the use of immunologic methods, including a specific antibody, to study cell constituents.

“Immunohistochemistry” refers to the use of immunologic methods, including a specific antibody, to study specific antigens in tissue slices.

“Immunolocalization” refers to the use of immunologic methods, including a specific antibody, to locate molecules or structures within cells or tissues.

“Immunologically active” refers to the capability of a natural, recombinant, or synthetic GPCR, or any immunogenic fragment thereof, to induce a specific immune response in appropriate animals or cells and to bind with specific antibodies. A polypeptide is “immunologically active” if it is recognized by (e.g., specifically bound by) a B-cell or T-cell surface antigen receptor. Immunological activity may generally be assessed using well known techniques, such as those summarized in Paul, Fundamental Immunology, 3rd ed., 243-247, Raven Press (1993) and references cited therein. Such techniques include screening polypeptides derived from the native polypeptide for the ability to react with antigen-specific antisera or T-cell lines or clones, which may be prepared in view of the present application using well known techniques. Preferably, an immunologically active portion of a GPCR protein reacts with such antisera or T-cells at a level that is not substantially lower than the reactivity of the full-length polypeptide (e.g., in an ELISA or T-cell reactivity assay). Such screens may generally be performed using methods well known to those of ordinary skill in the art in view of the present application, such as those described in Harlow and Lane, Antibodies: A Laboratory Manual, Cold Spring Harbor Press (1988). B-cell and T-cell epitopes may also be predicted via computer analysis.

“Immune response” refers to any of the body's immunologic reactions to an antigen such as antibody formation, cellular immunity, hypersensitivity, or immunological tolerance.

“Insertion” and “addition” when referring to a change in a nucleotide or amino sequence indicate the addition of one or more nucleotides or amino acid residues, respectively, to the sequence.

“In situ hybridization” refers to use of a nucleic acid probe, typically a DNA or RNA probe, to detect the presence of a DNA or RNA sequence in target cells such as cloned bacterial cells, cultured eukaryotic cells, or tissue samples. In situ hybridization can also be used for locating genes on chromosomes. The process can be performed by preparing a microscope slide with cells in metaphase of mitosis, then treating slide with a weak base to denature the DNA. Next, pour radioactively labeled probe onto the slide under hybridizing conditions, expose the slide to a photographic emulsion for a suitable period such as a few days or weeks, then develop the emulsion.

“Isoform” refers to different forms of a protein that may be produced from different genes or from the same gene by alternative RNA splicing.

“Isolated” generally means that the material is removed from its original environment (e.g., the natural environment if it is naturally occurring).

“Library” refers physically to a pool of nucleic acid fragments that has been propagated in a cloning vector. Library can also refer to an electronic collection of genomic or proteomic sequence data, including raw sequences, contigs, ORFs and loci from a specific organism.

“Ligand” refers to an ion or molecule that binds with another molecule, such as a GPCR, to form a macromolecule such as a receptor-ligand complex. An “endogenous ligand” refers to a native ligand that binds to the receptor of the GPCR and modulates biological activity or functionality of the GPCR in its native environment. A “specific ligand” is a ligand able to bind to a particular GPCR and modulate the biological activity or functionality of the particular GPCR; an endogenous ligand is one example of a specific ligand.

“Microarray” refers to an array of distinct nucleic acid or amino acid molecules arrayed on a substrate, such as paper, nylon or any other type of membrane, filter, chip, glass slide, or any other suitable solid support. Microarrays can also refer to tissue microarrays, composed of small tissue pieces arranged on a slide. U.S. Pat. No. 5,143,854 and PCT Patent Publication Nos. WO 90/15070 and 92/10092.

“Mimetic” refers to a molecule, e.g., a peptide or non-peptide agent, such as a small molecule, that is able to perform the same biological activity as a certain biologically active agent. For example, some mimetics are molecules comprising the same biological function or activity as the particular GPCR. The structure of the mimetic can be developed from knowledge of the structure of the particular GPCR or portions thereof. For appropriate mimetics, the mimetic is able to effect some or all of the actions of a given antigenic peptide or antibodies against the angtigenic peptide. Such mimetics can be made, in view of the present application, using techniques well known in the art, see, e.g., U.S. Pat. Nos. 6,197,752; 6,093,697; 6,207,643; 5,849,323, and can be included in the various processes, methods, and systems, etc., described herein, such as databases, binding partner assays, probes, medicaments, and therapeutics.

“Modulate” refers to controllably changing the activity of a substance or other item, such as the biological activity of a GPCR, antigenic peptide or corresponding antibody. For example, modulation may cause an increase or a decrease in protein activity, binding characteristics, or other biological, functional, or immunological properties of the GPCR.

“Monoclonal antibody” refers to an antibody obtained from a population of substantially homogeneous antibodies, e.g., the individual antibodies comprising the population are identical except for possible naturally occurring mutations that may be present in minor amounts. Monoclonal antibodies include “chimeric” antibodies (immunoglobulins) in which a portion of the heavy or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al., P.N.A.S. USA, 81:6851-6855 (1984). Monoclonal antibodies are highly specific, being directed against a single antigenic site. As a matter of distinction, polyclonal antibody preparations typically include different antibodies directed against different determinants (epitopes) of a target antigen whereas each monoclonal antibody is directed against a single determinant on the antigen. Monoclonal antibodies can be synthesized by hybridoma culture, uncontaminated by other immunoglobulins. For example, the monoclonal antibodies to be used in accordance with the present invention may be made by the hybridoma method first described by Kohler and Milstein, Nature, 256:495 (1975), or may be made by recombinant DNA methods. See, e.g., U.S. Pat. No. 4,816,567. Monoclonal antibodies may also be isolated from phage antibody libraries using the techniques described in Clackson et al., Nature, 352:624-628 (1991), and Marks et al., J. Mol. Biol., 222:581-597 (1991), for example. The modifier “monoclonal” indicates the character of the antibody as being obtained from a substantially homogeneous population of antibodies, and is not to be construed as requiring production of the antibody by any particular method.

“Nonconservative” changes to an amino acid sequence, see Analog.

“Northern blotting” or “Northern analysis” refers to a method used to detect specific RNA sequences. For example, the process can be performed by electrophoresing RNA in a denaturing agarose gel, transferring the gel onto a membrane, and hybridizing with a labeled RNA or DNA probe.

“Nucleic acid sequence” refers to a polymer comprising a string of “nucleic acids” such as an oligonucleotide, or a polynucleotide or fragment thereof. The nucleic acid sequence can be from DNA or RNA of genomic or synthetic origin, may be single-stranded or double-stranded, and may represent the sense or the antisense strand. A nucleic acid sequence can also be a PNA or a DNA-like or RNA-like material. Unless stated otherwise, the term encompasses nucleic acids containing known analogues or mimetics of natural nucleotides that have similar binding properties as the reference nucleic acid.

“Oligonucleotide” refers to a nucleic acid sequence, generally between 6 nucleotides to 60 nucleotides, preferably about 15 to 30 nucleotides, and most preferably about 20 to 25 nucleotides, that can, for example, be used in PCR or other nucleic acid amplification or in a hybridization assay or microarray. “Oligonucleotide” includes “amplimers,” “primers,” “oligomers,” and “probes,” as these terms are commonly defined in the art. Oligonucleotides can be chemically synthesized. Such synthetic oligonucleotides may have no 5′ phosphate and if so will not ligate to another oligonucleotide without adding a phosphate, typically by using an ATP in the presence of a kinase. A synthetic oligonucleotide will ligate to a fragment that has not been dephosphorylated.

“Operably linked” or “operably connected” indicates that one element of an apparatus, system, or method, etc., is connected to another element of the apparatus, system, or method, etc., such that the two elements are able to perform their intended purposes. For example, when a promoter is linked to a polynucleotide to allow transcription of the polynucleotide, it is “operably linked” to the polynucleotide.

“Orphan receptor” refers to a receptor for which the endogenous ligand or other ligands inducing biological activity are not known.

“PCR” or “polymerase chain reaction” refers to an in vitro method that uses oligonucleotide primers, enzymes, and a series of repetitive temperature cycles to generate millions of copies of a nucleic acid, typically DNA, from an original specimen of a specific DNA sequence, which specimen may be present only in a trace amount.

“Plasmids” refers to extrachromasomal genetic elements composed of DNA or RNA found in both eukaryotic and prokaryotic cells that can propagate themselves autonomously in cells. Plasmids can be used as carriers or vectors to clone DNA molecules. They are designated by a lower case p preceded or followed by capital letters or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan in view of the present application.

“Polynucleotide encoding a polypeptide” indicates a polynucleotide that includes only the coding sequence for the polypeptide as well as polynucleotides that include additional coding or non-coding sequence.

“Portion” or “fragment” with regard to a protein (as in “a portion of a given protein”) refers to parts of that protein, a subsequence of the complete amino acid sequence of the receptor containing at least about 8, usually at least about 12, more typically at least about 20, and commonly at least about 30 or more contiguous amino acid residues, up to the entire amino acid sequence minus one amino acid. Thus, a protein “comprising at least a portion of the amino acid sequence of SEQ ID NO:XX” or a protein “comprising at least a portion of the amino acid sequence of a particular GPCR” encompasses the full-length protein and fragments thereof. A portion or fragment of a nucleic acid refers to nucleic acid sequences that are greater than about 12 nucleotides in length, and typically at least about 60 or 100 nucleotides, generally at least about 1000 nucleotides, or at least about 10,000 nucleotides in length, up to the entire nucleic acid sequence minus one nucleic acid.

“P-value” is a statistical term used to indicate the probabilty that an event is due to random chance. When used in reference to a result of BLAST searches, the number indicates the probability that a match between two sequences is due to random chance.

“Receptor” refers to a molecular structure, typically within a cell or on a cell surface, that selectively binds a specific substance (a ligand) and a specific physiologic effect that accompanies the binding. GPCRs are a type of cell-surface receptor, which means a protein in, on, or traversing the cell membrane (in the case of GPCRs, traversing the cell membrane) that recognizes and binds to specific molecules in the surrounding fluid. The binding to a receptor may serve to transport molecules into the cell's interior or to signal the cell to respond in some way.

“Recombinant” refers to both a method of production and a structure. Some recombinant nucleic acids and proteins are made by the use of recombinant DNA techniques that involve human intervention, either in manipulation or selection. Others are made by fusing two fragments that are not naturally contiguous to each other. Engineered vectors are encompassed, as well as nucleic acids comprising sequences derived using any synthetic oligonucleotide process.

“Sample” is used in its usual broad sense. For example, a biological sample suspected of containing nucleic acids encoding the GPCR, or fragments thereof, or the GPCR itself, may comprise a bodily fluid; an extract from a cell, chromosome, organelle, or membrane from a cell; a cell; genomic DNA, RNA, or cDNA (in solution or bound to a solid support); a tissue; a tissue print, and the like. Biological sample refers to samples from a healthy individual as well as to samples from a subject suspected of having or susceptible to having, e.g., immune-related diseases, cell growth-related diseases, cell regeneration-related diseases, immunological-related cell proliferative diseases, and autoimmune diseases. Examples of specific diseases include AIDS, allergies, Alzheimer's disease, amyotrophic lateral sclerosis, atherosclerosis, bacterial, fungal, protozoan and viral infections, benign prostatic hypertrophy, bone diseases (e.g., osteoarthritis, osteoporosis), carcinoma (e.g., basal cell carcinoma, breast carcinoma, embryonal carcinoma, ovarian carcinoma, renal cell carcinoma, lung adenocarcinoma, lung small cell carcinoma, pancreatic carcinoma, prostate carcinoma, transitional carcinoma of the bladder, squamous cell carcinoma, thyroid carcinoma), cardiomyopathy, chronic and acute inflammation, circadian rhythm disorders, COPD, Crohn's disease, diabetes, Duchenne muscular dystrophy, embryonal carcinoma, endotoxic shock, environmental stress (e.g., by heat, UV or chemicals), gastrointestinal disorders, glioblastoma multiform, graft vs. host disease, Hodgkin's disease, inflammatory bowel disease, ischemia, stroke, lymphoma, macular degeneration, malignant cytokine production, malignant fibrous histiocytoma, melanoma, meningioma, mesothelioma, multiple sclerosis, nasal congestion, pain, Parkinson's disease, prostate carcinoma, psoriasis, rhabdomyosarcoma, psychotic or neurological disorders (e.g., anxiety, depression, schizophrenia, dementia, mental retardation, memory loss, epilepsy, locomotor problems, respiratory disorders, asthma, eating/body weight disorders including obesity, bulimia, diabetes, anorexia, nausea, hypertension, hypotension), renal disorders, reperfusion injury, rheumatoid arthritis, sarcoma (e.g., chondrosarcoma, Ewing's sarcoma, osteosarcoma), septicemia, seminoma, sexual/reproductive disorders, tonsil, transitional carcinoma of the bladder, transplant rejection, trauma, tuberculosis, ulcers, ulcerative colitis, urinary retention, vascular and cardiovascular disorders, or any other disease or disorder in which G protein-coupled receptors are involved, as well as learning and/or memory disorders, diabetes, pain perception disorders, anorexia, obesity, hormonal release problems, or any other disease or disorder in which a specific GPCR is involved.

“Second messengers” refer to intracellular signaling molecules such as cyclic AMP (cAMP), inositol triphosphate, diacylglycerol, or Ca²⁺. Second messengers, in turn, alter the activity of other intracellular proteins such as cAMP-dependent protein kinase and Ca²⁺/calmodulin-dependent protein kinases, leading to the transduction and amplification of the original extracellular signal.

“Southern blotting” refers to a method for detecting specific DNA sequences via hybridization. For example, a DNA sample can be electrophoresed in a denaturing agarose gel, transferred onto a membrane, and hybridized with a complementary nucleic acid probe. “Southern” when used in reference to a database indicates an electronic analog of the laboratory technique, which analysis can be used to identify libraries in which a given DNA sequence, such as a gene, EST, or ORF is present. The terms “Northern” and “Western” likewise can be used for electronic analogs to the respective laboratory techniques described above.

“Specific binding” or “specifically binding” refers to an interaction between protein or peptide and a certain substance, such as its specific ligand or antibody, and in some cases its agonists or antagonists. The interaction is dependent upon the presence of a particular structure of the protein recognized by the binding molecule (e.g., the antigenic determinant or epitope). For example, if an antibody specifically binds epitope “A,” the presence of a polypeptide containing epitope A or the presence of free unlabeled epitope A will reduce the amount of labeled epitope A that binds to the antibody in a reaction containing free labeled epitope A and the antibody. Conversely, the presence of a polypeptide that does not contain epitope A will not reduce the amount of labeled epitope A that binds to the antibody. Highly specific binding indicates that the protein or peptide binds to its particular ligand, antibody, etc., and does not bind in a significant amount to other proteins present in the sample. Typically, a specific or selective reaction will be at least twice the background signal or noise and more typically more than 10 to 100 times the background signal or noise.

“Stringent conditions” refer to conditions that permit hybridization between complementary polynucleotide sequences. Suitably stringent conditions can be defined by, for example, the concentrations of salt or formamide in the prehybridization and hybridization solutions, or by the hybridization temperature. Stringency can be increased by reducing the concentration of salt, increasing the concentration of formamide, or raising the hybridization temperature. Stringent conditions are dependent upon the type of probe as well as the length of the probe and the GC content of the probe. “Stringent conditions” typically occur within a range from about Tm-5° C. (5° C. below the melting temperature (Tm) of the probe) to about Tm-20-25° C. for a cRNA probe and to about Tm-15° C. for an oligonucleotide probe. “Highly stringent conditions” refers to conditions under which a probe will hybridize to its target sequence, typically in a complex mixture of nucleic acid sequences, but will not substantially hybridize to other sequences. One example of high stringency conditions for a cRNA probe that is 1,000 nucleotides in length and has a GC content of about 60% is about 55-65° C. in 50% formamide, 0.1×SSC, and 200 μg/ml sheared and denatured salmon sperm DNA. One example of low stringency conditions for the same probe in 50% formamide, 0.1×SSC, and 200 μg/ml sheared and denatured salmon sperm DNA would be 30-35° C. “Very highly stringent conditions” indicates that there must be complete identity between the sequences. The temperature range corresponding to a particular level of stringency can be narrowed further by calculating the purine to pyrimidine ratio of the nucleic acid of interest and adjusting the temperature accordingly. Variations on and modifications of the above ranges and conditions will be readily appreciated by those of skill in the art in view of the present application. As will be understood by those of skill in the art in view of the present application, the stringency of hybridization can be altered to identify or detect identical or related polynucleotide sequences. One guide for nucleic acid hybridization is Tijssen, Laboratory Techniques in Biochemistry and Molecular Biology-v.24 Hybridization with Nucleic Acid Probes, Part I “Overview of principles of hybridization and the strategy of nucleic acid assays” (New York: Elsevier 1993).

“Substantially purified” refers to nucleic acid or amino acid sequences that are removed from their natural environment and are separated from other components from such natural environment, and are at least about 60% free, preferably about 75% or 85% free, and most preferably about 90%, 95% or 99% free from such other components with which they are naturally associated. Substantially purified preferably indicates a substantially homogeneous state and can be in either a dry or aqueous solution or other composition as desired. Purity and homogeneity can be assayed by standard methods, for example on a mass or molar basis, using analytical chemistry techniques such as polyacrylamide gel electrophoresis or high performance liquid chromatography.

“Substitution” when referring to a change in a nucleotide or amino sequence indicates the replacement of one or more nucleotides or amino acids by different nucleotides or amino acids, respectively.

“Variant,” see Analog.

“Western blotting” or “Western analysis” refers to a method for detecting specific protein sequences. For example, the process can be performed by electrophoresing a protein mixture in a denaturing agarose or acrylamide gel, transferring the mixture onto a membrane, and incubating it with an antibody raised against the protein of interest.

Other terms and phrases are defined in other portions of this application.

C. Selection of Desired Antigenic Peptides for GPCRs and Other Polypeptides

The present invention provides improved antigenic peptides, for example as set forth in FIG. 2, SEQ ID NOS. 692-2292, and improved methods of identifying such antigenic peptides from known or publicly available sequences of polypeptides or proteins, i.e., from a candidate polypeptide sequence. Polypeptide and protein are used in their traditional sense to indicate lengthy amino acid molecules, whereas the antigenic peptide has a length significantly less than the length of the corresponding polypeptide or protein such that the antigenic peptide is capable of providing significantly improved antigenicity relative to the corresponding polypeptide or protein, typically improved specificity, affinity or avidity. The candidate polypeptide can be, for example, a human protein or polypeptide, a naturally occurring protein or polypeptide or a synthetic or recombinant protein or polypeptide.

The antigenic peptides are typically 5 to about 100 amino acids in length, preferably 6 to about 50 amino acids, and further preferably 7 to about 20 amino acids. The antigenic peptides include short antigenic amino acid sequences (i.e., peptides comprising only a portion of an antigenic sequence as set forth in FIG. 2 or as identified using the methods described herein, plus an insignificant number of additional amino acids at one or both ends, where insignificant indicates that the extra amino acids do not substantially interfere with the antigenicity of the antigenic peptide). Such short antigenic peptides can be identical to at least 5, 6, 7 or more consecutive amino acids of the sequences herein or identified using the methods described herein, or can have one or two (or more, with increasing length) conservative amino acid substitution for antigenic peptides comprising more than 6 or 7 consecutive amino acids of the sequences herein or identified using the methods described herein. Antigenic peptides and sequences, and related antibodies and assays and the like, are discussed further elsewhere herein with regard to GPCRs, but such discussions applies to all antigenic peptides produced according to the methods herein, including proteins and polypeptides such as kinases, phosphatases and any other desired protein or polypeptide.

The identification or selection methods comprise searching the candidate polypeptide sequence using a comparison window of the desired length, then selecting against or rejecting amino acid sequences of the length and having at least 1 characteristic selected from the group consisting of 1) at least two consecutive prolines, 2) at least two consecutive serines, 3) at least two consecutive lysines, 4) at least two consecutive arginines, 5) at least two consecutive aspartic acids, 6) at least two consecutive glutamic acids, 7) methionine, 8) tryptophan, and 9) at least five consecutive amino acids comprising no charged amino acids. Preferably, at least 5, 7, 8, or all of the characteristics are selected.

The identification or selection methods can also comprise selecting against amino acid sequences having at least 5 consecutive amino acids that are identical to an alternative amino acid sequence from an alternative polypeptide, i.e., some polypeptide other than the candidate polypeptide from which the selected antigen was derived, that is different from the candidate polypeptide, posttranslational modification sites, or highly hydrophobic sequences, which indicates sequences adequately hydrophobic to be located in a lipid membrane such as a cellular membrane. The posttranslational modification sites can be phosphorylation or glycosylation sites.

The methods can further comprise performing a BLAST-type or a FAST-type analyses for the candidate polypeptide sequence. Exemplary BLAST-type and FAST-type analyses are described above, including BLAST, BLASTP, BLASTX, FASTA, and FASTX.

D. General Discussion of Antigenic Peptides Related to Particular GPCRs

Antigenic Peptides Generally:

The present invention includes antigenic peptides able to induce specific immunogenic responses, and corresponding binding partners. Such antigenic peptides and binding partners can be cloned, expressed, isolated, purified, and otherwise obtained or manipulated according to routine methods known in the art in view of the present application.

The present invention further relates to antigenic peptides having an amino acid sequence from a particular GPCR, including analogs, mimetics, fragments, derivatives, and the like of such antigenic peptides. See SEQ ID NOS. 1-2292, FIGS. 1-3. The antigenic peptides may be recombinant, natural or synthetic. The antigenic peptides include (i) antigenic peptides in which one or more of the amino acid residues are substituted with a conserved or non-conserved amino acid residue (preferably a conserved amino acid residue) and such substituted amino acid residue may or may not be one encoded by the genetic code, (ii) antigenic peptides in which one or more of the amino acid residues includes a substituent group, (iii) antigenic peptides in which the mature polypeptide is complexed (e.g., fused or otherwise bonded) with another compound, such as a compound to increase the half-life of the polypeptide (for example, polyethylene glycol), and (iv) antigenic peptides in which additional amino acids are fused to the antigenic peptide. Preparing and using such analogs, etc., are within the scope of those skilled in the art in view of the present application. The antigenic peptides additionally include antigenic peptides that have at least about 90% identity to the given antigenic peptide, and preferably at least about 95% identity to the antigenic peptide. The antigenic peptides additionally include antigenic peptides that contain at least five, six, seven or more consecutive amino acids that are identical to the given antigenic peptide, as well as antigenic peptides that contain at least six, seven, eight or more consecutive amino acids that are identical to the given antigenic except for one or two conservative changes within this such stretch of amino acids. The antigenic peptides of the present invention can be produced by peptide synthesis.

Expression Profiles Based on Proteins:

An expression profile of a particular GPCR in one or more tissues can be made using antibodies or other binding partners produced using the antigenic peptides herein, then using traditional approaches such as Western blotting, immunohistochemistry analysis, protein array, ligand-binding studies, radioimmunoassay (RIA), and high performance liquid chromatography (HPLC), and immunohistochemistry analysis. H&E staining and other analyses can be used in combination with such immunologically-based analyses.

Screening for Activity:

The activity or functionality of an antigenic peptide can be measured using any of a variety of assays known in the art. Similarly, the specificity or affinity of an antibody or other binding partner made using the antigenic peptide can be measured using any of a variety of assays known in the art

The activity or functionality of a particular GPCR may be measured using any of a variety of functional assays in which activation of the receptor in question results in an observable change in the level of some second messenger system, including but not limited to adenylyl cyclase, calcium mobilization, arachidonic acid release, ion channel activity, inositol phospholipid hydrolysis, or guanylyl cyclase. Heterologous expression systems utilizing appropriate host cells to express the nucleic acid of the subject invention are used to obtain the desired second messenger coupling. Receptor activity may also be assayed in an oocyte expression system.

Protein Purification:

The antigenic peptides and proteins or polypeptides containing them can be purified by standard methods, including but not limited to salt or alcohol precipitation, preparative disc-gel electrophoresis, isoelectric focusing, high pressure liquid chromatography (HPLC), reversed-phase HPLC, gel filtration, cation and anion exchange, partition chromatography, and countercurrent distribution. Suitable purification methods will be readily apparent to those skilled in the art in view of the present application and are disclosed, e.g., in Guide to Protein Purification, Methods in Enzymology, Vol. 182, M. Deutscher, Ed., Academic Press, New York, N.Y. (1990). Purification steps can be followed as part of carrying out assays for ligand binding activity. Particularly where a particular GPCR is being isolated from a cellular or tissue source, it is preferable to include one or more inhibitors of proteolytic enzymes in the assay system, such as phenylmethylsulfonyl fluoride (PMSF).

E. Certain Assays, Antibodies, Probes, Therapeutics, and Other Systems and Aspects, of the Invention

1. Systems and Methods for Screening for a Particular GPCR or Antigenic Peptide

Screening for Antigenic Peptides:

As noted elsewhere herein, the present invention provides antigenic peptides and antibodies that are specific for a particular GPCR. The invention also provides systems and methods for using or detecting such peptides, and antibodies against such peptides or corresponding GPCRs in a sample. The assays are based on the detection of the antigenic peptides, typically as they are displayed by the particular GPCR, or the detection of antibodies produced against the particular antigenic peptides and corresponding GPCRs.

Screening for/with Antigenic Peptides:

Many assays are characterized by the ability of antigenic peptides for a particular GPCR to be bound by antibodies against them, and the ability of antibodies produced against such antigenic peptides to bind to antigens or epitopes of the particular GPCR in a sample. Some exemplary assays are described below and elsewhere herein.

List of Assays:

A variety of assays can detect antibodies that bind specifically to the desired protein in or from a sample, or detect a desired protein bound to one or more antibodies in or from the sample. Exemplary assays are described in detail in Antibodies: A Laboratory Manual, Harlow and Lane (eds.), Cold Spring Harbor Laboratory Press (1988). Representative examples of such assays include: countercurrent immuno-electrophoresis (CIEP), radioimmunoassays, radioimmunoprecipitations, enzyme-linked immunosorbent assays (ELISA), dot blot assays, inhibition or competition assays, sandwich assays, immunostick (dip-stick) assays, simultaneous assays, immunochromatographic assays, immunofiltration assays, latex bead agglutination assays, immunofluorescent assays, biosensor assays, and low-light detection assays. See U.S. Pat. Nos. 4,376,110 and 4,486,530; WO 94/25597; WO/25598.

Enzyme-Linked Immunosorbent Assays (ELISA):

One assay for the detection of a particular GPCR is a sandwich assay such as an enzyme-linked immunosorbent assay (ELISA). In one preferred embodiment, the ELISA comprises the following steps: (1) coating the particular GPCR antigenic peptide onto a solid phase, (2) incubating a sample suspected of containing anti-particular GPCR antibodies with the antigenic peptide coated onto the solid phase under conditions that allow the formation of an antigen-antibody complex, (3) adding an anti-antibody (such as anti-IgG) conjugated with a label to be captured by the resulting antigen-antibody complex bound to the solid phase, and (4) measuring the captured label and determining therefrom whether the sample contains anti-particular GPCR antibodies.

Immunofluorescence Assay:

A fluorescent antibody test (FA-test) uses a fluorescently labeled antibody able to bind to one of the proteins of the invention. For detection, visual determinations are made by a technician using fluorescence microscopy, yielding a qualitative result. In one embodiment, this assay is used for the examination of tissue samples or histological sections.

Bead Agglutination Assays:

In latex bead agglutination assays, antibodies to one or more of the antigenic peptides of the present invention are conjugated to latex beads. The antibodies conjugated to the latex beads are then contacted with a sample under conditions permitting the antibodies to bind to desired proteins in the sample, if any. The results are then read visually, yielding a qualitative result. In some embodiments, as with certain other assays, this format can be used in the field for on-site testing.

Enzyme Immunoassays:

Enzyme immunoassays (EIA) include a number of different assays that can use the antibodies described in the present application. For example, a heterogeneous indirect EIA uses a solid phase coupled with an antibody of the invention and an affinity purified, anti-IgG immunoglobulin preparation. The solid phase can be a polystyrene microtiter plate. The antibodies and immunoglobulin preparation are then contacted with the sample under conditions permitting antibody binding, which conditions are well known in the art. The results of such an assay can be read visually or using a device such as a spectrophotometer, such as an ELISA plate reader, to yield a quantitative result. An alternative solid phase EIA format includes plastic-coated ferrous metal beads able to be moved during the procedures of the assay by means of a magnet. Yet another alternative is a low-light detection immunoassay format. In this highly sensitive format, the light emission produced by appropriately labeled bound antibodies are quantified automatically. Preferably, the reaction is performed using microtiter plates.

In an alternative embodiment, a radioactive tracer is substituted for the enzyme-mediated detection in an EIA to produce a radioimmunoassay (RIA).

Sandwich Assay:

In a capture-antibody sandwich enzyme assay, the desired protein is bound between an antibody attached to a solid phase, preferably a polystyrene microtiter plate, and a labeled antibody. The results can be measured, for example, using a spectrophotometer, such as an ELISA plate reader.

Sequential and Simultaneous Assays:

In a sequential assay format, reagents are allowed to incubate with the capture antibody in a stepwise fashion. The test sample is first incubated with the capture antibody. Following a wash step, incubation with the labeled antibody occurs. In a simultaneous assay, the two incubation periods described in the sequential assay are combined. This eliminates one incubation period plus a wash step.

Immunostick (Dip-Stick) Assays:

A dipstick/immunostick format is essentially an immunoassay using a polystyrene paddle or dipstick instead of a polystyrene microtiter plate as the solid phase. Reagents are the same and the format can either be simultaneous or sequential.

Immunochromatographic Assays:

In a chromatographic strip test format, a capture antibody and a labeled antibody are dried onto a chromatographic strip, which typically comprises nitrocellulose or high porosity nylon bonded to cellulose acetate. The capture antibody is usually spray dried as a line at one end of the strip. At this end, there is an absorbent material that is in contact with the strip. At the other end of the strip, the labeled antibody is deposited in a manner that prevents it from being absorbed onto the membrane. Usually, the label attached to the antibody is a latex bead or colloidal gold. The assay may be initiated by applying the sample immediately in front of the labeled antibody.

Immunofiltration Assays:

Immunofiltration/immunoconcentration formats combine a large solid-phase surface with directional flow of sample/reagents, which concentrates and accelerates the binding of antigen to antibody. In an exemplary format, the test sample is preincubated with a labeled antibody, and then applied to a solid phase such as fiber filters, nitrocellulose membranes, or the like. The solid phase can also be precoated with latex or glass beads coated with capture antibody. Detection of analyte is the same as that in a standard immunoassay. The flow of sample/reagents can be modulated by either vacuum or the wicking action of an underlying absorbent material.

Biosensor Assays:

A threshold biosensor assay is a sensitive, instrumented assay amenable to screening large numbers of samples at low cost. In one embodiment, such an assay comprises the use of light-addressable potentiometric sensors wherein the reaction involves the detection of a pH change due to binding of the desired protein by capture antibodies, bridging antibodies, and urease-conjugated antibodies. Upon binding, a pH change is effected that is measurable by translation into electrical potential (μvolts). The assay typically occurs in a very small reaction volume, and is very sensitive; the reported detection limit of the assay is 1,000 molecules of urease per minute.

2. Antibodies

Antibodies Generated Against a Particular Antigenic Peptide and its Corresponding GPCR:

Highly specific, high affinity or antibodies against a particular GPCR or other polypeptide can be generated using the antigenic peptides herein and using antibody generation techniques as described herein or elsewhere. The antibodies produced using the antigenic peptides of the present invention, for example, have a specificity for the corresponding GPCR such that the antibodies can selectively detect the corresponding GPCR in a sample containing non-desired or contaminating proteins or polypeptides, such as a tissue or blood sample. Preferably, the antibodies have a high specificity such that no significant amounts of such proteins or polypeptides are detected, and further preferably have a specificity such that only insubstantial to essentially zero amounts of non-desirable proteins are detected. The antibodies produced using the antigenic peptides of the present invention, for example, typically have an affinity or avidity constant (Ka) of at least about 10⁷ liters/mole, typically a high affinity or avidity at least about 10⁹ liters/mole, preferably at least about 10¹⁰ liters/mole, and further preferably at least about 10¹¹ liters/mole.

The antibodies can be used to conduct immunohistochemistry and other analyses of a variety of tissue samples to determine expression of a particular GPCR in such tissues, for diagnostic assays, and for other desired purposes. The specification will now discuss a variety of antibody types, methods, uses, etc.

Antibodies Generally:

In some embodiments, the present invention provides antibodies and other binding partners created using the antigenic peptides herein and directed to a particular GPCR from which the antigenic peptides were derived. Compositions and uses for such antibodies are contemplated, including diagnostic, medicament, and therapeutic uses. Various diagnostic, medicament, and therapeutic uses for antibodies have been reviewed above and, for example, in Goldenberg et al., Semin. Cancer Biol., 1(3):217-225 (1990); Beck et al., Semin. Cancer Biol., 1(3):181-188 (1990); Niman, Immunol. Ser., 53:189-204 (1990); Endo, Nippon Igaku Hoshasen Gakkai Zasshi (Japan), 50(8):901-909 (1990); and, U.S. Pat. No. 6,214,984.

Recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. An exemplary immunoglobulin (antibody) structural unit comprises a tetramer. Each tetramer is composed of two identical pairs of antigenic peptide chains, each pair having one “light” chain (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Anti-Idiotypic Antibodies:

The present invention encompasses anti-idiotypic antibodies, including polyclonal and monoclonal anti-idiotypic antibodies, that are produced using the antibodies described herein as antigens. These anti-idiotypic antibodies are useful because they may mimic the structures of the antigenic peptides set forth herein.

Techniques for producing antibodies, including antibody fragments, include the following.

a. Antibody Preparation

(i) Polyclonal Antibodies

Antibody Prep—Polyclonal:

Polyclonal antibodies are generally raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor, using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCL₂, or R¹N═C═NR, where R and R¹ are different alkyl groups.

Antibody Prep—Adjuvants (All ABS):

Suitable adjuvants for the vaccination of animals for the production of polyclonal, monoclonal, and other antibodies include but are not limited to Adjuvant 65 (containing peanut oil, mannide monooleate, and aluminum monostearate); Freund's complete or incomplete adjuvant; mineral gels such as aluminum hydroxide, aluminum phosphate, and alum; surfactants such as hexadecylamine, octadecylamine, lysolecithin, dimethyldioctadecylammonium bromide, N,N-dioctadecyl-N′,N′-bis(2-hydroxymethyl) propanediamine, methoxyhexadecylglycerol, and pluronic polyols; polyanions such as pyran, dextran sulfate, poly IC, polyacrylic acid, and carbopol; peptides such as muramyl dipeptide, dimethylglycine, tuftsin, stress proteins, core-containing proteins from a positive stranded RNA virus, see U.S. Pat. No. 6,153,378; and, oil emulsions. The antigenic peptides could also be administered following incorporation into liposomes or other microcarriers.

Information concerning adjuvants and various aspects of immunoassays are disclosed, e.g., in the series by P. Tijssen, Practice and Theory of Enzyme Immunoassays, 3rd Edition (1987), Elsevier, New York. Other useful references covering methods for preparing polyclonal antisera include Microbiology, Hoeber Medical Division, Harper and Row (1969); Landsteiner, Specificity of Serological Reactions, Dover Publications, New York (1962); and, Williams, et al., Methods in Immunology and Immunochemistry, Vol. 1, Academic Press, New York (1967).

Animals can be immunized against the antigen, immunogenic conjugates, or derivatives by combining 1 mg or 1 μg of the peptide or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein or through a different cross-linking reagent. Conjugates also can be made in recombinant cell culture as protein fusions. In addition, aggregating agents such as alum can be suitably used to enhance the immune response.

(ii) Monoclonal Antibodies

Antibody Prep—Monoclonal:

Monoclonal antibodies are obtained from a population of substantially homogeneous antibodies, e.g., the individual antibodies comprising the population are identical except for possible naturally occurring mutations that may be present in minor amounts. For example, monoclonal antibodies can be made using the hybridoma method first described by Kohler and Milstein, Nature, 256:495 (1975), or can be made by recombinant DNA methods, or otherwise as desired.

In the hybridoma method, a mouse, or other appropriate host animal, such as a hamster, is immunized as described herein to elicit lymphocytes that produce or are capable of producing antibodies that will bind specifically to the antigenic peptide used for immunization. Alternatively, lymphocytes may be immunized in vitro. Lymphocytes then are fused with myeloma cells using a suitable fusing agent, such as polyethylene glycol, to form a hybridoma cell, Goding, Monoclonal Antibodies: Principles and Practice, pp. 59-103, Academic Press (1986).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium, for example murine myeloma lines, such as those derived from MOPC-21 and MPC-11 mouse tumors available from the Salk Institute Cell Distribution Center, San Diego, Calif. USA, and SP-2 cells available from the American Type Culture Collection, Rockville, Md. USA. Human myeloma and mouse-human heteromyeloma cell lines have also been described for the production of human monoclonal antibodies, Kozbor, J. Immunol., 133:3001 (1984); Brodeur et al., Monoclonal Antibody Production Techniques and Applications, pp. 51-63, Marcel Dekker, Inc., New York (1987).

Culture medium in which hybridoma cells are growing is assayed for production of monoclonal antibodies directed against the antigenic peptide. The binding specificity of monoclonal antibodies produced by hybridoma cells can be determined by immunoprecipitation or by an in vitro binding assay, such as radioimmunoassay (RIA) or enzyme-linked immunosorbent assay (ELISA). The binding affinity of the monoclonal antibody can, for example, be determined by the Scatchard analysis of Munson and Pollard, Anal. Biochem., 107:220 (1980). The antibodies produced using the antigenic peptides of the present invention, for example, typically have an affinity or avidity constant (Ka) of at least about 10⁷ liters/mole, typically a high affinity or avidity at least about 10⁹ liters/mole, preferably at least about 10¹⁰ liters/mole, and further preferably at least about 10¹¹ liters/mole.

After hybridoma cells are identified that produce antibodies of the desired specificity, affinity, or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-SEPHAROSE™, hydroxyapatite chromatography, gel electrophoresis, dialysis, or affinity chromatography.

DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which can then be transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Review articles on recombinant expression in bacteria of DNA encoding antibody include Skerra et al., Curr. Opinion in Immunol., 5:256-262 (1993), and Pluckthun, Immunol. Revs., 130:151-188 (1992).

Moabs—Combinatorial:

In a further embodiment, antibodies or antibody fragments can be isolated from antibody phage libraries generated using the techniques described in McCafferty et al., Nature, 348:552-554 (1990), using the proper antigen such as CD1 la, CD18, IgE, or HER-2 to select for a suitable antibody or antibody fragment. Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling, Marks et al., Biotechnology, 10:779-783 (1992), as well as combinatorial infection and in vivo recombination as strategies for constructing very large phage libraries, Waterhouse et al., Nuc. Acids. Res., 21:2265-2266 (1993). Combinatorial antibodies are also discussed in Huse et al., Science 246:1275-1281 (1989), and Sastry et al., Proc. Natl. Acad. Sci. USA, 86:5728-5732 (1989), and Alting-Mees et al., Strategies in Molecular Biology 3:1-9 (1990). These references describe a system commercially available from Stratacyte, La Jolla, Calif. USA. Briefly, mRNA is isolated from a B cell population and utilized to create heavy and light chain immunoglobulin cDNA expression libraries in the λIMMUNOZAP(H) and λIMMUNOZAP(L) vectors. These vectors may be screened individually or co-expressed to form Fab fragments or antibodies, see Huse et al., supra; see also Sastry et al., supra. Positive plaques can subsequently be converted to a non-lytic plasmid, which allows for high-level expression of monoclonal antibody fragments from E. coli.

Humanized Moab:

Binding partners can also be constructed utilizing recombinant DNA techniques to incorporate the variable regions of a gene that encode a specifically binding antibody. The construction of these binding partners can be readily accomplished by one of ordinary skill in the art in view of the present application. See Larrick et al., Biotechnology, 7:934-938 (1989); Riechmann et al., Nature, 332:323-327 (1988); Roberts et al., Nature, 328:731-734 (1987); Verhoeyen et al., Science 239:1534-1536 (1988); Chaudhary et al., Nature, 339:394-397 (1989); see also U.S. Pat. No. 5,132,405 entitled “Biosynthetic Antibody Binding Sites”.) For example, the DNA can be modified by substituting the coding sequence for human heavy- and light-chain constant domains in place of homologous murine sequences, U.S. Pat. No. 4,816,567; Morrison, et al., Proc. Nat. Acad. Sci., 81:6851 (1984), or by covalently joining to the immunoglobulin coding sequence all or part of the coding sequence for a non-immunoglobulin polypeptide. In another example, DNA segments encoding the desired antigen-binding domains specific for the protein or peptide of interest are amplified from appropriate hybridomas and inserted directly into the genome of a cell that produces human antibodies. See Verhoeyen et al., supra; see also Reichmann et al., supra. Some of these techniques transfer the antigen-binding site of a specifically binding mouse or rat monoclonal antibody or the like to a human antibody. Such antibodies can be preferable for therapeutic use in humans because they are typically not as antigenic as rat or mouse antibodies.

In an alternative embodiment, genes that encode the variable region from a hybridoma producing a monoclonal antibody of interest can be amplified using oligonucleotide primers for the variable region. These primers may be synthesized by one of ordinary skill in the art, or may be purchased from commercially available sources. For instance, primers for mouse and human variable regions including, among others, primers for V_{H}a, V_{H}b, V_{H}c, V_{H}d, C_{H}1, V_{L}, and C_{L} regions are available from Stratacyte (La Jolla, Calif.). These primers may be utilized to amplify heavy- or light-chain variable regions, which may then be inserted into vectors such as IMMUNOZAP™(H) or IMMUNOZAP™(L) (Stratacyte), respectively. These vectors may then be introduced into E. coli for expression. Utilizing these techniques, large amounts of a single-chain protein containing a fusion of the VH and VL domains may be produced, see Bird et al., Science 242:423-426 (1988).

Antibody Substitutions—Non-Immunoglobulin Polypeptides (All ABS):

Non-immunoglobulin polypeptides can be substituted in monoclonal and other antibodies described herein for the constant domains of an antibody, or they can be substituted for the variable domains of one antigen-combining site of an antibody to create a chimeric bivalent antibody comprising one antigen-combining site having specificity for an antigen and another antigen-combining site having specificity for a different antigen.

CHIMERICS:

Chimeric or hybrid antibodies can also be prepared in vitro using known methods in synthetic protein chemistry, including those involving crosslinking agents, in view of the present application. For example, immunotoxins may be constructed using a disulfide-exchange reaction or by forming a thioether bond. Examples of suitable reagents for this purpose include iminothiolate and methyl-4-mercaptobutyrimidate.

Antibody Labeling (All ABS):

For diagnostic applications or otherwise as desired, and for monoclonal and other antibodies described herein, the antibodies and other binding partners typically will be labeled with a detectable moiety. The detectable moiety can be any moiety that is capable of producing, either directly or indirectly, a detectable signal. For example, the detectable moiety may be a radioisotope, such as ³H, ¹⁴C, ³²P, ³⁵S, or ¹²⁵I; a fluorescent or chemiluminescent compound, such as fluorescein isothiocyanate, rhodamine, or luciferin; or an enzyme, such as alkaline phosphatase, beta-galactosidase, or horseradish peroxidase. Any method known in the art for conjugating the antibody or binding partner to the detectable moiety may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. Cytochem., 30:407 (1982).

(iii) Humanized And Human Antibodies

Humanized AB Generally:

Methods for humanizing non-human antibodies are well known in the art and have been discussed in part above. Generally, a humanized antibody has one or more amino acid residues introduced into it from a source which is non-human. These non-human amino acid residues are often referred to as “import” residues, which are typically taken from an “import” variable domain. Humanization can be performed essentially following the method of Winter and co-workers, Jones et al., Nature, 321:522-525 (1986); Riechmann et al., Nature, 332:323-327 (1988); Verhoeyen et al., Science, 239:1534-1536 (1988), by substituting rodent CDRs or CDR sequences for the corresponding sequences of a human antibody. Accordingly, such humanized antibodies are chimeric antibodies, U.S. Pat. No. 4,816,567, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making humanized antibodies is very important to reduce antigenicity. According to the so-called “best-fit” method, the sequence of the variable domain of a rodent antibody is screened against the entire library of known human variable-domain sequences. The human sequence that is closest to that of the rodent is then accepted as the human framework (FR) for the humanized antibody. Sims et al., J. Immunol., 151:2296 (1993); Chothia and Lesk, J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Natl. Acad. Sci. USA, 89:4285 (1992); Presta et al., J. Immunol., 151:2623 (1993).

It is typically desirable that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind antigen. In this way, FR residues can be selected and combined from the consensus and import sequences so that the desired antibody characteristic, such as increased affinity for the target antigen(s), is achieved. In general, CDR residues are directly and most substantially involved in influencing antigen binding.

It is also possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA. 90:2551-255 (1993); Jakobovits et al., Nature, 362:255-258 (1993); Bruggemann et al., Year Immuno., 7:33 (1993). Human antibodies can also be produced in phage-display libraries, Hoogenboom and Winter, J. Mol. Biol., 227:381 (1991); Marks et al., J. Mol. Biol., 222:581 (1991).

(iv) Antibody Fragments

Antibody Fragments:

Various techniques have been developed for the production of antibody fragments. Such fragments can be derived via proteolytic digestion of intact antibodies, see, e.g., Morimoto et al., J. Biochem. Biophys. Meth. 24:107-117 (1992) and Brennan et al., Science, 229:81 (1985). Fragments can also be produced directly by recombinant host cells. For example, antibody fragments can be isolated from antibody phage libraries discussed above. Fab′-SH fragments can be directly recovered from E. coli and chemically coupled to form F(ab′)₂ fragments, Carter et al., Biotechnology 10:163-167 (1992). F(ab′)₂ fragments can be isolated directly from recombinant host cell culture. Other techniques for the production of antibody fragments will be apparent to the skilled practitioner.

(v) Bispecific Antibodies

Bispecific Antibodies Generally:

Bispecific antibodies (BsAbs) are antibodies that have binding specificities for at least two different antigens. Bispecific antibodies can be derived from full-length antibodies or from antibody fragments, e.g., F(ab′)₂ bispecific antibodies.

Methods for making bispecific antibodies are known in the art. Traditional production of full-length bispecific antibodies is based on the coexpression of two immunoglobulin heavy chain-light chain pairs, where the two chains have different specificities, Millstein and Cuello, Nature, 305:537-539 (1983). Because of the random assortment of immunoglobulin heavy and light chains, these hybridomas (quadromas) produce a mixture of potentially 10 different antibody molecules, of which only one has the correct bispecific structure. Purification of the correct molecule, which is usually accomplished by affinity chromatography steps, is rather cumbersome, and the product yields are low. Similar procedures are disclosed in WO 93/08829, and in Traunecker et al., E.M.B.O. J., 10:3655-3659 (1991).

According to another approach, antibody variable domains containing the desired binding specificities (antibody-antigen combining sites) are fused to immunoglobulin constant domain sequences. The fusion is preferably with an immunoglobulin heavy chain constant domain, comprising at least part of the hinge, C_{H} 2, and C_{H} 3 regions. It is preferred to have the first heavy-chain constant region (C_{H} 1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the improved yields. It is, however, possible to insert the coding sequences for two or all three polypeptide chains in one expression vector when the expression of at least two polypeptide chains in equal ratios results in high yields or when the ratios are of no particular significance.

Antibodies—Hybrid Immunoglobulin Heavy Chain:

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure may facilitate the separation of the desired bispecific compound from unwanted immunoglobulin chain combinations, as the presence of an immunoglobulin light chain in only one half of the bispecific molecule provides for a facile method of separation. This approach is discussed in WO 94/04690. For further details of generating bispecific antibodies see, for example, Suresh et al., Meth. Enzymol., 121:210 (1986).

ANTIBODIES—CROSS-LINKED OR “HETEROCONJUGATE”:

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells, U.S. Pat. No. 4,676,980), and for treatment of HIV infection, WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

ANTIBODIES—DIABODIES:

The “diabody” technology described by Hollinger et al., Proc. Natl. Acad. Sci. USA, 90:6444-6448 (1993) has provided an alternative mechanism for making BsAb fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites.

Another strategy for making BsAb fragments by the use of single-chain Fv (sFv) dimers has also been reported. See Gruber et al., J. Immunol., 152:5368 (1994). These researchers designed an antibody comprising the V_{H} and V_{L} domains of a first antibody joined by a 25-amino-acid-residue linker to the V_{H} and V_{L} domains of a second antibody. The refolded molecule bound to fluorescein and the T-cell receptor and redirected the lysis of human tumor cells that had fluorescein covalently linked to their surface.

Antibodies—Other:

Techniques for generating bispecific antibodies from antibody fragments have also been described in the literature. For example, bispecific antibodies can be prepared using chemical linkage. Brennan et al., Science, 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the BsAb. The BsAbs produced can be used as agents for the selective immobilization of enzymes.

Fab′-SH fragments can be directly recovered from E. coli, which can be chemically coupled to form bispecific antibodies. Shalaby et al., J. Exp. Med., 175:217-225 (1992) describe the production of a fully humanized BsAb F(ab′)₂ molecule. Each Fab′ fragment was separately secreted from E. coli and subjected to directed chemical coupling in vitro to form the BsAb. The BsAb thus formed was able to bind to cells overexpressing the HER2 receptor and normal human T cells, as well as trigger the lytic activity of human cytotoxic lymphocytes against human breast tumor targets. See also Rodriguez et al., Int. J. Cancers (Suppl.) 7:45-50 (1992).

Various techniques for making and isolating BsAb fragments directly from recombinant cell culture have also been described. For example, bispecific F(ab′)₂ heterodimers have been produced using leucine zippers. Kostelny et al., J. Immunol., 148(5):1547-1553 (1992). The leucine zipper peptides from the Fos and Jun proteins are linked to the Fab′ portions of two different antibodies by gene fusion. The antibody homodimers are reduced at the hinge region to form monomers and then re-oxidized to form the antibody heterodimers. b. Antibody Purification 12211 ANTIBODY PURIFICATION GENERALLY:

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992), describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

Before LPHIC:

The antibody composition prepared from the cells is preferably subjected to at least one purification step prior to LPHIC. Examples of suitable purification steps include hydroxyapatite chromatography, gel electrophoresis, dialysis, and affinity chromatography. The suitability of protein A as an affinity ligand depends on the species and isotype of any immunoglobulin Fc domain that is present in the antibody. Protein A can be used to purify antibodies that are based on human γ1, γ2, or γ4 heavy chains, Lindmark et al., J. Immunol. Meth. 62:1-13 (1983). Protein G has been recommended for mouse isotypes and for human γ3, Guss et al., E.M.B.O. J., 5:1567-1575 (1986). The matrix to which the affinity ligand is attached is often agarose, but other matrices are available. Mechanically stable matrices such as controlled pore glass or poly(styrenedivinyl)benzene allow for faster flow rates and shorter processing times than can be achieved with agarose. Where the antibody comprises a C_{H} 3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™, chromatography on an anion or cation exchange resin (such as a polyaspartic acid column), chromatofocusing, SDS-PAGE, and ammonium sulfate precipitation are also available depending on the antibody to be recovered.

LPHIC:

Following any preliminary purification step(s), the mixture comprising the antibody of interest and contaminant(s) can be subjected to LPHIC. See U.S. Pat. No. 6,214,984. Often, the antibody composition to be purified will be present in a buffer from the previous purification step. However, it may be necessary to add a buffer to the antibody composition prior to the LPHIC step. Many buffers are available and can be selected by routine experimentation. The pH of the mixture comprising the antibody to be purified and at least one contaminant in a loading buffer is adjusted to a pH of about 2.5-4.5 using either an acid or base, depending on the starting pH. The loading buffer can have a low salt concentration (e.g., less than about 0.25 M salt).

The mixture is loaded on the HIC column. HIC columns normally comprise a base matrix (e.g., cross-linked agarose or synthetic copolymer material) to which hydrophobic ligands (e.g., alkyl or aryl groups) are coupled. One example of an HIC column comprises an agarose resin substituted with phenyl groups (e.g., a Phenyl SEPHAROSE™ column). Many HIC columns are available commercially. Examples include, but are not limited to, Phenyl SEPHAROSE 6 FAST FLOW™ column with low or high substitution (Pharmacia LKB Biotechnology, AB, Sweden); Phenyl SEPHAROSE™ High Performance column (Pharmacia LKB Biotechnology, AB, Sweden); Octyl SEPHAROSE™ High Performance column (Pharmacia LKB Biotechnology, AB, Sweden); FRACTOGEL™ TM EMD Propyl or FRACTOGEL™ EMD Phenyl columns (E. Merck, Germany); MACRO-PREP™ Methyl or MACRO-PREP™ t-Butyl Supports (Bio-Rad, California); WP HI-Propyl (C₃)™ column (J. T. Baker, New Jersey); and TOYOPEARL™ ether, phenyl, or butyl columns (TosoHaas, PA).

The antibody is typically eluted from the column using an elution buffer that is the same as the loading buffer. The elution buffer can be selected using routine experimentation in view of the present application. The pH of the elution buffer may be between about 2.5-4.5 and have a low salt concentration (e.g., less than about 0.25 M salt). It may not be necessary to use a salt gradient to elute the antibody of interest; the desired product may be recovered in the flow-through fraction that does not bind significantly to the column.

The LPHIC step provides a way to remove a correctly folded and disulfide bonded antibody from unwanted contaminants (e.g., incorrectly associated light and heavy fragments). The method can provide an approach to substantially remove an impurity characterized as a correctly folded antibody fragment whose light and heavy chains fail to associate through disulfide bonding. Antibody compositions prepared using LPHIC can be up to about 95% pure or more. Purities of more than about 98% have been reported. U.S. Pat. No. 6,214,984.

Post LPHIC:

Antibody compositions prepared by LPHIC can be further purified as desired using techniques which are well known in the art. Diagnostic or therapeutic formulations of the purified protein can be made by providing the antibody composition in a physiologically acceptable carrier, examples of which are provided below. To remove contaminants (e.g., unfolded antibody and incorrectly associated light and heavy fragments) from the HIC column so that it can be re-used, a composition including urea (e.g., 6.0 M urea, 1% MES buffer pH 6.0, 4 mM ammonium sulfate) can be flowed through the column.

c. Some Uses for Antibodies Described Herein

(i) Generally

Generally:

The present invention comprises any suitable use for the antibodies and other binding partners discussed herein. The following provides some of the desired uses, including diagnostic and therapeutic uses. Various diagnostic and therapeutic uses for antibodies have been reviewed in Goldenberg et al., Semin. Cancer Biol., 1(3):217-225 (1990); Beck et al., Semin. Cancer Biol., 1(3):181-188 (1990); Niman, Immunol. Ser. 53:189-204 (1990); and, Endo, Nippon Igaku Hoshasen Gakkai Zasshi (Japan) 50(8):901-909 (1990), for example.

Assays:

The antibodies can be used in immunoassays, such as enzyme immunoassays. BsAbs can be useful for this type of assay; one arm of the BsAb can be designed to bind to a specific epitope on the enzyme so that binding does not cause enzyme inhibition, the other arm of the antibody can be designed to bind to an immobilizing matrix ensuring a high enzyme density at the desired site. Examples of such diagnostic BsAbs include those having specificity for IgG as well as ferritin, and those having binding specificities for horseradish peroxidase (HRP) as well as a hormone, for example. Monoclonal and polyclonal antibodies are also exemplary antibodies for immunoassays.

The antibodies can be designed for use in two-site immunoassays. For example, two antibodies are produced binding to two separate epitopes on the analyte protein; one antibody binds the complex to an insoluble matrix, the other binds an indicator enzyme.

Diagnostic Uses:

Antibodies can also be used for immunodiagnosis, in vitro or in vivo or otherwise, of various diseases or conditions based on the presence or absence of a particular GPCR. Such diseases and conditions include, e.g., immune-related diseases, cell growth-related diseases, cell regeneration-related diseases, immunological-related cell proliferative diseases, and autoimmune diseases. Examples of specific diseases include AIDS, allergies, Alzheimer's disease, amyotrophic lateral sclerosis, atherosclerosis, bacterial, fungal, protozoan and viral infections, benign prostatic hypertrophy, bone diseases (e.g., osteoarthritis, osteoporosis), carcinoma (e.g., basal cell carcinoma, breast carcinoma, embryonal carcinoma, ovarian carcinoma, renal cell carcinoma, lung adenocarcinoma, lung small cell carcinoma, pancreatic carcinoma, prostate carcinoma, transitional carcinoma of the bladder, squamous cell carcinoma, thyroid carcinoma), cardiomyopathy, chronic and acute inflammation, circadian rhythm disorders, COPD, Crohn's disease, diabetes, Duchenne muscular dystrophy, embryonal carcinoma, endotoxic shock, environmental stress (e.g., by heat, UV or chemicals), gastrointestinal disorders, glioblastoma multiform, graft vs. host disease, Hodgkin's disease, inflammatory bowel disease, ischemia, stroke, lymphoma, macular degeneration, malignant cytokine production, malignant fibrous histiocytoma, melanoma, meningioma, mesothelioma, multiple sclerosis, nasal congestion, pain, Parkinson's disease, prostate carcinoma, psoriasis, rhabdomyosarcoma, psychotic or neurological disorders (e.g., anxiety, depression, schizophrenia, dementia, mental retardation, memory loss, epilepsy, locomotor problems, respiratory disorders, asthma, eating/body weight disorders including obesity, bulimia, diabetes, anorexia, nausea, hypertension, hypotension), renal disorders, reperfusion injury, rheumatoid arthritis, sarcoma (e.g., chondrosarcoma, Ewing's sarcoma, osteosarcoma), septicemia, seminoma, sexual/reproductive disorders, tonsil, transitional carcinoma of the bladder, transplant rejection, trauma, tuberculosis, ulcers, ulcerative colitis, urinary retention, vascular and cardiovascular disorders, or any other disease or disorder in which G protein-coupled receptors are involved, as well as learning and/or memory disorders, diabetes, pain perception disorders, anorexia, obesity, hormonal release problems, or any other disease or disorder in which a specific GPCR is involved.

To facilitate this diagnostic use, an antibody that binds a particular GPCR, when such is differentially expressed in tumors or other target diseases, can be conjugated with a detectable marker (e.g., a chelator that binds a radionuclide). Examples of tumor-associated antigens being used in a similar fashion include an antibody having specificity for the tumor-associated antigen CEA used for imaging colorectal and thyroid carcinomas and the anti-p185^{HER2} antibody used for detecting cancers characterized by amplification of the HER2 protooncogene. Other uses for the antibodies of the present invention will be apparent to the skilled practitioner in view of the present application.

(ii) Assays

Assays:

For certain applications such as some diagnostic and other assay applications, the antibody typically can be labeled directly or indirectly with a detectable moiety. The detectable moiety can be any moiety that is capable of producing, either directly or indirectly, a detectable signal. For example, the detectable moiety may be a radioisotope, such as ³H, ¹⁴C, ³²p, 35%, or 125I; a fluorescent or chemiluminescent compound, such as fluorescein isothiocyanate, rhodamine, or luciferin; or an enzyme, such as alkaline phosphatase, beta-galactosidase, or HRP.

Any method known in the art for separately conjugating the antibody to the detectable moiety may be employed, including those methods described by Hunter et al., Nature, 144:945 (1962); David et al., Biochemistry, 13:1014 (1974); Pain et al., J. Immunol. Meth. 40:219 (1981); and, Nygren, J. Histochem. and Cytochem. 30:407 (1982).

The antibodies of the present invention may be employed in any desired assay method, such as competitive binding assays, direct, and indirect sandwich assays, and immunoprecipitation assays. Zola, Monoclonal Antibodies: A Manual of Techniques, pp. 147-158 (CRC Press, Inc. (1987).

Competitive Binding Assays:

Competitive binding assays rely on the ability of a labeled standard to compete with the test sample analyte for binding with a limited amount of antibody. The amount of analyte in the test sample is inversely proportional to the amount of standard that becomes bound to the antibody. To facilitate determining the amount of standard that becomes bound, the antibody generally is insolubilized before or after the competition, so that the standard, and analyte that are bound to the antibody may conveniently be separated from the standard, and analyte which remain unbound.

BsAbs are particularly useful for sandwich assays which involve the use of two molecules, each capable of binding to a different immunogenic portion, or epitope, of the sample to be detected. In a sandwich assay, the test sample analyte is bound by a first arm of the antibody which is immobilized on a solid support, and thereafter a second arm of the antibody binds to the analyte, thus forming an insoluble three part complex. See, e.g., U.S. Pat. No. 4,376,110. The second arm of the antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme. Assays are discussed further elsewhere herein in relation to binding partners such as antibodies, and antigenic peptides for particular GPCRs, including assays searching for or using such antigenic peptides, and would be apparent to those skilled in the art in view of the present application.

(iii) Affinity Purification

Affinity Purification:

The antibodies also are useful for the affinity purification of an antigen of interest such as a particular GPCR from sources such as recombinant cell culture or natural sources.

(iv) Therapeutics

Therapeutic Uses:

Therapeutic compositions, and uses, etc., for the antibodies described herein will now be discussed. As with other parts of this application, this section does not contain the entire discussion of therapeutic uses or compositions, etc., for antibodies; other sections discuss both antibodies, and therapeutics, and the discussion in this section applies to certain other aspects discussed herein. Turning to antibodies and therapeutics, the antibodies can be used, for example, for redirected cytotoxicity (e.g., to kill tumor cells), as a vaccine adjuvant, for delivering thrombolytic agents to clots, for delivering immunotoxins to tumor cells, for converting enzyme activated prodrugs at a target site (e.g., a tumor), for treating infectious diseases or targeting immune complexes to cell surface receptors.

Therapeutic Formulations:

Therapeutic formulations of the antibody can be prepared for storage by mixing the antibody having the desired degree of purity with optional physiologically acceptable carriers, excipients, or stabilizers (Remington's Pharmaceutical Sciences, 16th edition, Osol, A., Ed. (1980), for example in the form of lyophilized cake or aqueous solutions. Acceptable carriers, excipients, or stabilizers are nontoxic to recipients at the dosages, and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; or nonionic surfactants such as Tween, Pluronics, or polyethylene glycol (PEG).

The antibodies also may be entrapped in microcapsules prepared, for example, by coacervation techniques or by interfacial polymerization (for example, hydroxymethylcellulose or gelatin-microcapsules, and poly-[methylmethacrylate] microcapsules, respectively), in colloidal drug delivery systems (for example, liposomes, albumin microspheres, microemulsions, nano-particles, and nanocapsules), or in macroemulsions. Such techniques are disclosed in Remington's Pharmaceutical Sciences, supra.

Therapeutic Formulations—Sterile:

An antibody to be used for in vivo human administration should be sterile. This can be accomplished by filtration through sterile filtration membranes, for example prior to or following lyophilization and reconstitution. The antibody ordinarily will be stored in lyophilized form or in solution. Therapeutic antibody compositions generally are placed into a container having a sterile access port, for example, an intravenous solution bag or vial having a stopper pierceable by a hypodermic injection needle.

Therapeutic Administrations:

The route of antibody administration is in accord with known methods, e.g., injection or infusion by intravenous, intraperitoneal, intracerebral, intramuscular, intraocular, intraarterial, or intralesional routes, or by sustained release systems as noted below.

The antibody can be administered, for example, continuously by infusion or by bolus injection. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15:167-277 (1981), and Langer, Chem. Tech., 12:98-105 (1982), or poly(vinylalcohol)), polylactides, U.S. Pat. No. 3,773,919; EP 58,481, copolymers of L-glutamic acid and gamma ethyl-L-glutamate, Sidman et al., Biopolymers, 22:547-556 (1983), non-degradable ethylene-vinyl acetate, Langer et al., supra, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid, EP 133,988.

Therapeutic Administrations—Sustained Release-Polymers:

While polymers such as ethylene-vinyl acetate and lactic acid-glycolic acid sustain release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for antibody stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Therapeutic Administrations—Sustained Release-Liposomes:

Sustained-release antibody compositions also include liposomally entrapped antibody. Liposomes containing the antibody can be prepared by methods such as those in DE 3,218,121; Epstein et al., Proc. Natl. Acad. Sci. USA, 82:3688-3692 (1985); Hwang et al., Proc. Natl. Acad. Sci. USA, 77:4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamellar type in which the lipid content is greater than about 30 mol. % cholesterol, the selected proportion being adjusted for the optimal antibody therapy.

Therapeutically Effective Amount:

An effective amount of antibody to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. A typical daily dosage might range from about 1 μg/kg to up to 10 mg/kg or more, depending on the factors mentioned above. Typically, the clinician will administer antibody until a dosage is reached that achieves the desired effect. The progress of this therapy is easily monitored by conventional assays.

5. Drug Design Based on the Antigens Herein or Antibodies Thereto

Disease/Conditions List:

The peptides and antibodies of the present invention can serve as valuable tools for designing drugs for treating various pathophysiological conditions such as immune-related diseases, cell growth-related diseases, cell regeneration-related diseases, immunological-related cell proliferative diseases, and autoimmune diseases. Examples of specific diseases include AIDS, allergies, Alzheimer's disease, amyotrophic lateral sclerosis, atherosclerosis, bacterial, fungal, protozoan and viral infections, benign prostatic hypertrophy, bone diseases (e.g., osteoarthritis, osteoporosis), carcinoma (e.g., basal cell carcinoma, breast carcinoma, embryonal carcinoma, ovarian carcinoma, renal cell carcinoma, lung adenocarcinoma, lung small cell carcinoma, pancreatic carcinoma, prostate carcinoma, transitional carcinoma of the bladder, squamous cell carcinoma, thyroid carcinoma), cardiomyopathy, chronic and acute inflammation, circadian rhythm disorders, COPD, Crohn's disease, diabetes, Duchenne muscular dystrophy, embryonal carcinoma, endotoxic shock, environmental stress (e.g., by heat, UV or chemicals), gastrointestinal disorders, glioblastoma multiform, graft vs. host disease, Hodgkin's disease, inflammatory bowel disease, ischemia, stroke, lymphoma, macular degeneration, malignant cytokine production, malignant fibrous histiocytoma, melanoma, meningioma, mesothelioma, multiple sclerosis, nasal congestion, pain, Parkinson's disease, prostate carcinoma, psoriasis, rhabdomyosarcoma, psychotic or neurological disorders (e.g., anxiety, depression, schizophrenia, dementia, mental retardation, memory loss, epilepsy, locomotor problems, respiratory disorders, asthma, eating/body weight disorders including obesity, bulimia, diabetes, anorexia, nausea, hypertension, hypotension), renal disorders, reperfusion injury, rheumatoid arthritis, sarcoma (e.g., chondrosarcoma, Ewing's sarcoma, osteosarcoma), septicemia, seminoma, sexual/reproductive disorders, tonsil, transitional carcinoma of the bladder, transplant rejection, trauma, tuberculosis, ulcers, ulcerative colitis, urinary retention, vascular and cardiovascular disorders, or any other disease or disorder in which G protein-coupled receptors are involved, as well as learning and/or memory disorders, diabetes, pain perception disorders, anorexia, obesity, hormonal release problems, or any other disease or disorder in which a specific GPCR is involved or that would be readily apparent to those skilled in the art in view of the present application.

### EXAMPLES

The Examples below provide information as follows: Example 1 relates to the identification and selection of the antigens set forth in FIG. 2. Examples 2 to 4 relate to antibody production and purification based on such antigens. Examples 5 to 10 relate to H&E staining. And, Example 11 relates to Western blot analyses.

### Example 1

### Selection of Antigens

Antigenic peptides were derived from the amino acid sequence of a particular GPCR based on analyses of likely antigen-containing regions and specificity of those regions for the protein/gene of interest. The specificity of the antigen peptides (approximately 20 amino acids in length) for antibody generation was determined using the outlined techniques, including BLAST of several public databases. These public databases included but were not limited to GenBank, Swiss Prot Human, Swiss Prot NonHuman, GenPeptH, GenPept M, and LifeSpan's proprietary databases. With respect to specificity, parameters that precluded the use of a particular peptide included the presence of 6 or more contiguous amino acids with sequence identity to protein(s) other than the protein of interest, the presence of sites of posttranslational modification, including phosphorylation and glycosylation, and highly hydrophobic sequences, which could indicate potential in situ localization within the plasma membrane. The peptides were analyzed for antigenicity using the published algorithm of Hopp, T. P., and Woods, K. R, Proc. Natl. Acad. Sci. U.S.A. 78, 3824-3828, (1981). Additional considerations in antigenic peptide design included 1) selection against sequences with multiple prolines in a row, 2) selection against sequences with multiple serines in a row, 3) selection against sequences with multiple lysines in a row, 4) selection against sequences with multiple arginines in a row 5) selection against sequences with multiple aspartic acids in a row, 6) selection against sequences with multiple glutamic acids in a row, 7) selection against peptides containing methionine or tryptophan, which can become oxidized as a result of the cyclization reaction, and 8) avoidance of stretches of 5 or more amino acids having no uncharged amino acids (which also resulted in a desirable charge to peptide length ratio of at least 1 charge: 5 residues). The selected antigenic peptides are set forth in the Sequence Listing and in FIG. 2.

### Example 2

### Antibody Production Schedule

Day 0—Pre-immune serum collection (approximately 5.0 ml). Immunize using 200 μg antigen peptide per rabbit in Complete Freund's Adjuvant.

Day 14—Immunize using 100 μg antigen per rabbit in Incomplete Freund's Adjuvant.

Day 28—Immunize using 100 μg antigen per rabbit in Incomplete Freund's Adjuvant.

Day 42—Immunize using 100 μg antigen per rabbit in Incomplete Freund's Adjuvant.

Day 49—First production bleed; obtain 24.0-26.0 ml.

Day 56—Immunize using 100 μg antigen per rabbit in Incomplete Freund's Adjuvant.

Day 63—Second production bleed and ELISA analysis.

Day 70—Immunize using 100 μg antigen per rabbit in Incomplete Freund's Adjuvant.

Day 77—Third production bleed and affinity purification.

### Example 3

### Immunosorbent Purification of Antiserum: Coupling of Peptide to CNBr-Activated Sepharose 4

Weigh out 0.8 g of CNBr-activated Sepharose 4B (2.5 ml of final gel volume). Wash and re-swell on sintered glass filter with 1 mM HCl, followed by coupling buffer (0.1 M NaHCO₃, 0.25 M NaCl, pH 8.5). Dissolve 10 mg of protein or peptide in coupling buffer. Mix protein solution with gel suspension and incubate 2 hours at room temperature or overnight at 4° C. Block remaining active groups with 0.2 M glycine buffer, pH 8.1. Wash away excess adsorbed protein with coupling buffer, followed by 0.1 M acetate buffer containing 0.5 M NaCl, pH 4.3. Equilibrate the column with phosphate-buffered saline (PBS), pH 7.7.

### Example 4

### Immunosorbent Purification of Antiserum Affinity Purification of Antiserum

Dilute 10 ml of clear antiserum 1:1 with PBS, pH 7.7, apply to affinity column at a flow rate of 0.3 ml/minute, and monitor absorbance of eluate at 280 nm. Collect fractions of unbound material and rinse column with PBS, pH 7.7. Elute bound antibody with 0.2 M glycine, pH 1.85, and collect eluate until absorbance at 280 nm returns to baseline. Neutralize all collected fractions with 1 M Tris-HCl, pH 8.5 immediately after collection. Determine OD at 280 nm, and determine the total OD recovered. Conduct ELISA analysis with the corresponding antigen to confirm the presence and identity of recovered antibody and the removal of all antibody from the original serum. Concentrate antibody to approximately 2.0 mg/ml and dialyze against PBS with 0.01% NaN₃.

### Example 5

### Preparation of Antibody Dilutions

The purpose of this protocol is to dilute antibodies in solution. Materials include Tris-HCl Buffer with carrier protein and 0.015 M NaN₃ (Dako Antibody Diluent #S0809 (DAKO, Carpentaria, Calif.); vials containing the antibodies described above or commercial antibodies against the particular GPCR; pipetmen and disposable tips; container of chopped ice; 12 ml Dako reagent tubes; and, reagent tube rack.

The procedure is a) calculate proportions of antibody and diluent according to desired concentrations and volume requirements; b) label reagent tubes and place in rack; c) pipette needed volume of diluent into tube(s); d) place vials of antibodies into ice; e) invert and/or flick antibody vial(s) 3 or 4 times to insure suspension; f) pipette required volume of antibody(s) into corresponding diluent volumes; and, g) mix gently.

### Example 6

### Preparation of Autostainer Solutions

The purpose of this protocol is the preparation of concentrated solutions for use in a DAKO autostainer. Materials include DAKO® TBST (Tris Buffered Saline Containing Tween-S3306), 10× Concentrate, DAKO® Target Retrieval Solution, 10× Concentrate (S1699), deionized H₂O, 20L container, with lid, marked at the 10L level, DAKO® TBS (Tris Buffered Saline-S 1968), and DAKO Tween® (S 1966).

The procedure to make TBST 10× Concentrate is a) pour 2 500 ml bottles DAKO® TBST into a 20 L container, b) add deionized H₂O until solution level is at 10 L mark, c) replace lid and shake 10 to 20 times, d) pour diluted DAKO® TBST into autostainer carboy(s) as designated. The procedure to make Target Retrieval Solution is a) measure 135 ml of deionized H₂O and pour into slide bath, b) measure 15 ml of DAKO® Target Retrieval solution, c) add to H₂O, and d) agitate. This solution is then used in the steam method of target retrieval, Example 9, below. The procedure to make TBS is a) fill 20L container to 10L mark with deionized H₂O, b) add 2 envelopes of DAKO® TBS, c) add 5 ml of DAKO TWEEN®, and d) replace lid and agitate 10 to 20 times.

### Example 7

### Preparation of Solutions for Antibody Detection

Solutions for antibody detection are prepared using Vector® Biotinylated antibody (BA series), Vectastain® ABC-AP Kit (AK-5000), 10 mM sodium phosphate, pH 7.5, 0.9% saline (PBS), Vector® Red Alkaline Phosphatase Substrate Kit I (SK-5100), and 100 mM Tris-HCl, pH 8.2 Buffer. To prepare biotinylated antibody, add 10 ml of PBS to reagent tube, add 1 drop biotinylated antibody to the PBS, then mix gently. To prepare ABC, to 10 ml of PBS, add 2 drops each of Reagent A and Reagent B, mix immediately, then allow to stand 30 minutes before use. To prepare AP Red, which should be prepared immediately before use, to 5 ml of Tris-HCl buffer, add 2 drops of Reagent 1 and mix well, add 2 drops of Reagent 2 and mix well, then add 2 drops of Reagent 3 and mix well.

### Example 8

### Deparaffinization and Rehydration of Samples

The purpose of this protocol is to remove paraffin from and rehydrate preserved tissues in preparation for IHC procedures. Materials and equipment include fume hood, vertical slide rack(s), three xylene (VWR #72060-088) baths, three 100% alcohol blend (VWR #72060-050) baths, two 95% alcohol blend (VWR #72060-052) baths, one 70% alcohol blend (VWR #72060-056) bath, and Tris-Buffered Saline (DAKO® S1968)+Tween® (DAKO S1966).

Insert the slides into the vertical rack(s). Move slides through baths inside fume hood as follows:

Xylene 5 Minutes

Xylene 5 Minutes

Xylene 5 Minutes

100% Alcohol 2 Minutes

100% Alcohol 2 Minutes

100% Alcohol 1 Minute

95% Alcohol 2 Minutes

95% Alcohol 2 Minutes

70% Alcohol 1 Minute

Finally, place slides into a container with TBST.

### Example 9

### Steam Method of Target Retrieval

The purpose of this protocol is to optimize antibody binding within paraffin embedded tissues. Materials and equipment included a steamer, deionized H₂O, target retrieval solution, 10× concentrate (DAKO #S 1699), 250 ml graduated cylinder, 15 ml graduated cylinder, staining dish(es), and deparaffinized and rehydrated tissue on microscope slides in immersed TBST. The procedure is to a) fill the steamer with deionized H20 to appropriate depth as indicated, b) turn the steamer on, c) in a graduated cylinder, measure 135 ml of deionized H₂O and pour into staining dish(es), d) pipette 15 ml of target retrieval solution and release into deionized H₂O, e) place the staining dish(es) into the basket of the steamer and heat for at least 10 minutes to preheat, f) add rack(s) containing tissue slides to heated target retrieval solution, g) cover and steam for 20 minutes, h) remove container from steamer and let stand at room temperature for 20 minutes, i) transfer rack(s) with slides to container(s) of TBST, and j) slides are now ready for staining procedures.

### Example 10

### Antibody Detection

The deparaffinized, rehydrated, and steamed (if needed) slides are loaded onto racks within a DAKO autostainer and then the autostainer is run according to the manufacturer's instructions. The slides are removed and the autostainer is turned off.

### Example 11

### Western BLOTTING

The purpose of this protocol is to visualize the immunoreactivity of the antibodies described above against the particular GPCR on a western blot. Materials and equipment included western blot membrane, TBS Tween (TBST: 100 mM Tris-HCl pH 7.5, 150 mM NaCl, 0.1% Tween™ 20), 5% non-fat dried milk in TBST (blotto), antibody of interest (primary), peroxidase-conjugated AffiniPure goat anti-rabbit IgG (H+L) (secondary)—Jackson ImmunoResearch, ECL solution (Amersham Biosciences, Uppsala Sweden), film, developer D-19, fixer, rocking platform.

During the blotting procedure, the blot is kept wet at all times and on a substantially level surface. The Western blot is placed right-side up in 10 ml of blotto. The membrane is flipped over and the dish rocked so that the solution covered it. The membrane is then flipped back to the right side and solution is again rocked over it. The blot is then placed on a shaker for at least 1 hour. Ten ml of primary antibody are prepared by diluting 1:500 in blotto.

The blotto is removed from the Western blot and replaced with the primary antibody. The blot is flipped again and placed on the shaker for 1 hour. Secondary antibody and peroxidase-conjugated AffiniPure goat anti-rabbit IgG (H+L) are prepared 1:20,000 in 10 ml of blotto. The primary antibody is removed and the Western blot is washed 3 times with 10 ml of blotto. The blotto is removed and replaced with the secondary antibody solution. The blot is flipped and placed on the shaker for 1 hour. The secondary antibody is removed and the blot washed 2 times with 10 ml of blotto. The blotto is removed and the blot is washed 2 times with 10 ml TBST. ECL is prepared by combining equal amounts of Solution 1 and 2.

The blotto is removed and 1 ml of ECL is placed on the blot. The blot is flipped and let sit for 1 minute. The blot is placed on plastic wrap and immediately covered with plastic wrap. The ECL is pressed out. The blot is placed on the film, then the film is developed.

From the foregoing, it will be appreciated that, although specific embodiments of the invention have been described herein for purposes of illustration, various modifications may be made without deviating from the spirit and scope of the invention. Accordingly, the invention includes all permutations and combinations of the subject matter set forth herein and is not limited except as by the appended claims.

Sequence Listing:

Provided On Accompanying Diskette

What is claimed is:

1. An isolated antigenic peptide according to any one of SEQ ID NOS. 692-2292.

2. An isolated antigenic peptide comprising an amino acid sequence that is at least about 90% identical to a sequence set forth in any one of SEQ ID NOS. 692-2292.

3. An isolated antigenic peptide that is an analog of an antigenic peptide according to any one of SEQ ID NOS. 692-2292.

4. An isolated antigenic peptide comprising a short antigenic amino acid sequence that is identical to at least 5 consecutive amino acids set forth in any one of SEQ ID NOS. 692-2292.

5. An isolated antigenic peptide comprising a short antigenic amino acid sequence that is identical to or contains no more than one conservative amino acid substitution over at least 7 consecutive amino acids set forth in any one of SEQ ID NOS. 692-2292.

6. A kit for the detection of antibodies against a particular GPCR in a sample comprising:
a) an isolated antigenic peptide according to any one of claims 1-5 and derived from the particular GPCR, and
b) at least one of a reagent or a device for detecting the antibodies.

7. An isolated antibody having high specificity and high affinity or avidity for a particular GPCR comprising a peptide sequence that is identical to any one of SEQ ID NOS. 692-703, 713-730, 744-802, 807-820, 825-875, 880-889, 917-941, 950-964, 971-984, 989-993, 1010-1013, 1021-1024, 1029-1043, 1049-1052, 1057-1072, 1087-1113, 1124-1151, 1161-1172, 1179-1187, 1198-1209, 1228-1231, 1245-1257, 1271-1279, 1304-1308, 1369-1372, wherein the antibody was produced using an isolated antigenic peptide comprising the peptide sequence that is identical to the any one of SEQ ID NOS. 692-703, 713-730, 744-802, 807-820, 825-875, 880-889, 917-941, 950-964, 971-984, 989-993, 1010-1013, 1021-1024, 1029-1043, 1049-1052, 1057-1072, 1087-1113, 1124-1151, 1161-1172, 1179-1187, 1198-1209, 1228-1231, 1245-1257, 1271-1279, 1304-1308, 1369-1372.

8. An isolated antibody having high specificity and high affinity or avidity for a particular GPCR comprising a peptide sequence that is at least about 90% identical to any one of SEQ ID NOS. 692-703, 713-730, 744-802, 807-820, 825-875, 880-889, 917-941, 950-964, 971-984, 989-993, 1010-1013, 1021-1024, 1029-1043, 1049-1052, 1057-1072, 1087-1113, 1124-1151, 1161-1172, 1179-1187, 1198-1209, 1228-1231, 1245-1257, 1271-1279, 1304-1308, 1369-1372, wherein the antibody was produced using the peptide sequence that is at least about 90% identical to the any one of SEQ ID NOS. 692-703, 713-730, 744-802, 807-820, 825-875, 880-889, 917-941, 950-964, 971-984, 989-993, 1010-1013, 1021-1024, 1029-1043, 1049-1052, 1057-1072, 1087-1113, 1124-1151, 1161-1172, 1179-1187, 1198-1209, 1228-1231, 1245-1257, 1271-1279, 1304-1308, 1369-1372.

9. An isolated antibody having high specificity and high affinity or avidity for a particular GPCR comprising a peptide sequence that is an analog to any one of SEQ ID NOS. 692-703, 713-730, 744-802, 807-820, 825-875, 880-889, 917-941, 950-964, 971-984, 989-993, 1010-1013, 1021-1024, 1029-1043, 1049-1052, 1057-1072, 1087-1113, 1124-1151, 1161-1172, 1179-1187, 1198-1209, 1228-1231, 1245-1257, 1271-1279, 1304-1308, 1369-1372, wherein the antibody was produced using an isolated antigenic peptide comprising the peptide sequence that is the analog to the any one of SEQ ID NOS. 692-703, 713-730, 744-802, 807-820, 825-875, 880-889, 917-941, 950-964, 971-984, 989-993, 1010-1013, 1021-1024, 1029-1043, 1049-1052, 1057-1072, 1087-1113, 1124-1151, 1161-1172, 1179-1187, 1198-1209, 1228-1231, 1245-1257, 1271-1279, 1304-1308, 1369-1372.

10. An isolated antibody having high specificity and high affinity or avidity for a particular GPCR comprising a peptide sequence that is identical to at least 5 consecutive amino acids set forth any one of SEQ ID NOS. 692-703, 713-730, 744-802, 807-820, 825-875, 880-889, 917-941, 950-964, 971-984, 989-993, 1010-1013, 1021-1024, 1029-1043, 1049-1052, 1057-1072, 1087-1113, 1124-1151, 1161-1172, 1179-1187, 1198-1209, 1228-1231, 1245-1257, 1271-1279, 1304-1308, 1369-1372, wherein the antibody was produced using a short isolated antigenic peptide comprising the at least 5 consecutive amino acids set forth in the any one of SEQ ID NOS. 692-703, 713-730, 744-802, 807-820, 825-875, 880-889, 917-941, 950-964, 971-984, 989-993, 1010-1013, 1021-1024, 1029-1043, 1049-1052, 1057-1072, 1087-1113, 1124-1151, 1161-1172, 1179-1187, 1198-1209, 1228-1231, 1245-1257, 1271-1279, 1304-1308, 1369-1372.

11. An isolated antibody specific for a particular GPCR comprising a peptide sequence that is identical to any one of SEQ ID NOS. 704-712, 731-743, 774-777, 803-806, 821-824, 876-879, 890-916, 942-949, 965-970, 985-988, 994-1009, 1014-1020, 1025-1028, 1044-1048, 1053-1056, 1073-1086, 1114-1123, 1152-1160, 1173-1178, 1188-1197, 1210-1227, 1232-1244, 1258-1270, 1280-1303, 1309-1368, 1373-1377, 1386-1389, 1394-1402, 1462-1482, 1496-1525, 1542-1549, 1557-1563, 1583-1649, 1656-1679, 1684-1688, 1693-1732, 1744-1752, 1765-1839, 1846-1854, 1855-1866, 1871-1917, 1926-1941, 1952-1955, 1960-1980, 1985-2141, 2152-2165, and 2170-2292, wherein the antibody was produced using an isolated antigenic peptide comprising the peptide sequence that is identical to the any one of SEQ ID NOS. 704-712, 731-743, 774-777, 803-806, 821-824, 876-879, 890-916, 942-949, 965-970, 985-988, 994-1009, 1014-1020, 1025-1028, 1044-1048, 1053-1056, 1073-1086, 1114-1123, 1152-1160, 1173-1178, 1188-1197, 1210-1227, 1232-1244, 1258-1270, 1280-1303, 1309-1368, 1373-1377, 1386-1389, 1394-1402, 1462-1482, 1496-1525, 1542-1549, 1557-1563, 1583-1649, 1656-1679, 1684-1688, 1693-1732, 1744-1752, 1765-1839, 1846-1854, 1855-1866, 1871-1917, 1926-1941, 1952-1955, 1960-1980, 1985-2141, 2152-2165, and 2170-2292.

12. An isolated antibody specific for a particular GPCR comprising a peptide sequence that is at least about 90% identical to any one of SEQ ID NOS. 704-712, 731-743, 774-777, 803-806, 821-824, 876-879, 890-916, 942-949, 965-970, 985-988, 994-1009, 1014-1020, 1025-1028, 1044-1048, 1053-1056, 1073-1086, 1114-1123, 1152-1160, 1173-1178, 1188-1197, 1210-1227, 1232-1244, 1258-1270, 1280-1303, 1309-1368, 1373-1377, 1386-1389, 1394-1402, 1462-1482, 1496-1525, 1542-1549, 1557-1563, 1583-1649, 1656-1679, 1684-1688, 1693-1732, 1744-1752, 1765-1839, 1846-1854, 1855-1866, 1871-1917, 1926-1941, 1952-1955, 1960-1980, 1985-2141, 2152-2165, and 2170-2292, wherein the antibody was produced using the peptide sequence that is at least about 90% identical to the any one of SEQ ID NOS. 704-712, 731-743, 774-777, 803-806, 821-824, 876-879, 890-916, 942-949, 965-970, 985-988, 994-1009, 1014-1020, 1025-1028, 1044-1048, 1053-1056, 1073-1086, 1114-1123, 1152-1160, 1173-1178, 1188-1197, 1210-1227, 1232-1244, 1258-1270, 1280-1303, 1309-1368, 1373-1377, 1386-1389, 1394-1402, 1462-1482, 1496-1525, 1542-1549, 1557-1563, 1583-1649, 1656-1679, 1684-1688, 1693-1732, 1744-1752, 1765-1839, 1846-1854, 1855-1866, 1871-1917, 1926-1941, 1952-1955, 1960-1980, 1985-2141, 2152-2165, and 2170-2292.

13. An isolated antibody specific for a particular GPCR comprising a peptide sequence that is an analog to any one of SEQ ID NOS. 704-712, 731-743, 774-777, 803-806, 821-824, 876-879, 890-916, 942-949, 965-970, 985-988, 994-1009, 1014-1020, 1025-1028, 1044-1048, 1053-1056, 1073-1086, 1114-1123, 1152-1160, 1173-1178, 1188-1197, 1210-1227, 1232-1244, 1258-1270, 1280-1303, 1309-1368, 1373-1377, 1386-1389, 1394-1402, 1462-1482, 1496-1525, 1542-1549, 1557-1563, 1583-1649, 1656-1679, 1684-1688, 1693-1732, 1744-1752, 1765-1839, 1846-1854, 1855-1866, 1871-1917, 1926-1941, 1952-1955, 1960-1980, 1985-2141, 2152-2165, and 2170-2292, wherein the antibody was produced using an isolated antigenic peptide comprising the peptide sequence that is the analog to the any one of SEQ ID NOS. 704-712, 731-743, 774-777, 803-806, 821-824, 876-879, 890-916, 942-949, 965-970, 985-988, 994-1009, 1014-1020, 1025-1028, 1044-1048, 1053-1056, 1073-1086, 1114-1123, 1152-1160, 1173-1178, 1188-1197, 1210-1227, 1232-1244, 1258-1270, 1280-1303, 1309-1368, 1373-1377, 1386-1389, 1394-1402, 1462-1482, 1496-1525, 1542-1549, 1557-1563, 1583-1649, 1656-1679, 1684-1688, 1693-1732, 1744-1752, 1765-1839, 1846-1854, 1855-1866, 1871-1917, 1926-1941, 1952-1955, 1960-1980, 1985-2141, 2152-2165, and 2170-2292.

14. An isolated antibody specific for a particular GPCR comprising a peptide sequence that is identical to at least 5 consecutive amino acids set forth any one of SEQ ID NOS. 704-712, 731-743, 774-777, 803-806, 821-824, 876-879, 890-916, 942-949, 965-970, 985-988, 994-1009, 1014-1020, 1025-1028, 1044-1048, 1053-1056, 1073-1086, 1114-1123, 1152-1160, 1173-1178, 1188-1197, 1210-1227, 1232-1244, 1258-1270, 1280-1303, 1309-1368, 1373-1377, 1386-1389, 1394-1402, 1462-1482, 1496-1525, 1542-1549, 1557-1563, 1583-1649, 1656-1679, 1684-1688, 1693-1732, 1744-1752, 1765-1839, 1846-1854, 1855-1866, 1871-1917, 1926-1941, 1952-1955, 1960-1980, 1985-2141, 2152-2165, and 2170-2292, wherein the antibody was produced using a short isolated antigenic peptide comprising the at least 5 consecutive amino acids set forth in the any one of SEQ ID NOS. 704-712, 731-743, 774-777, 803-806, 821-824, 876-879, 890-916, 942-949, 965-970, 985-988, 994-1009, 1014-1020, 1025-1028, 1044-1048, 1053-1056, 1073-1086, 1114-1123, 1152-1160, 1173-1178, 1188-1197, 1210-1227, 1232-1244, 1258-1270, 1280-1303, 1309-1368, 1373-1377, 1386-1389, 1394-1402, 1462-1482, 1496-1525, 1542-1549, 1557-1563, 1583-1649, 1656-1679, 1684-1688, 1693-1732, 1744-1752, 1765-1839, 1846-1854, 1855-1866, 1871-1917, 1926-1941, 1952-1955, 1960-1980, 1985-2141, 2152-2165, and 2170-2292.

15. A kit for the detection of antibodies against the particular GPCR of claim 5 comprising:
a) an isolated antibody according to any one of claims 7-14, and
b) at least one of a reagent or a device for detecting the antibody.

16. An assay for the detection of a particular GPCR in a sample, comprising:
a) providing an isolated antigenic peptide according to any one of claims 1-5,
b) contacting the isolated antigenic peptide with the sample under conditions suitable and for a time sufficient for the antigenic peptide to bind to one or more antibodies specific for the particular GPCR present in the sample, to provide an antibody-bound antigenic peptide, and
c) detecting the antibody-bound antigenic peptide, and therefrom determining whether the sample contains the particular GPCR.

17. The assay of claim 16 further comprising the step of binding the isolated antigenic peptide or the antibody to a solid substrate.

18. The assay of claim 16 or 17 wherein the sample is an unpurified sample.

19. The assay of any one of claims 15-18 further comprising, prior to the contacting, obtaining the sample from a human being.

20. The assay of any one of claims 15-19 wherein the assay is selected from the group consisting of a countercurrent immuno-electrophoresis (CIEP) assay, a radioimmunoassay, a radioimmunoprecipitation, an enzyme-linked immuno-sorbent assay (ELISA), a dot blot assay, an inhibition or competition assay, a sandwich assay, an immunostick (dip-stick) assays, a simultaneous assay, an immunochromatographic assay, an immunofiltration assay, a latex bead agglutination assay, an immunofluorescent assay, a biosensor assay, and a low-light detection assay.

21. An isolated nucleic acid molecule encoding an antigenic peptide according to any one of SEQ ID NOS. 692-2292.

22. The isolated nucleic acid molecule according to claim 21 wherein the molecule encodes a naturally occurring human antigenic peptide.

23. An isolated nucleic acid molecule encoding an antigenic peptide that is at least about 90% identical to any one of the antigenic peptides set forth in SEQ ID NOS. 692-2292.

24. The isolated nucleic acid molecule according to claim 23 wherein the antigenic peptide is at least about 95% identical to the antigenic peptide.

25. The isolated nucleic acid molecule according to claim 23 or 24 wherein the molecule encodes a naturally occurring human antigenic peptide.

26. A process for producing an isolated polynucleotide comprising hybridizing a nucleotide encoding an antigenic peptide according to any one of SEQ ID NOS. 692-2292 to genomic DNA under highly stringent conditions and isolating the polynucleotide detected with the nucleotide.

27. A method of identifying an amino acid sequence for an antigenic peptide from a candidate polypeptide sequence wherein the antigenic peptide has a length of about 5 to about 100 amino acids, the method comprising:
a) searching the candidate polypeptide sequence using a comparison window of the length, and
b) selecting against amino acid sequences of the length and having at least 3 characteristics selected from the group consisting of 1) at least two consecutive prolines, 2) at least two consecutive serines, 3) at least two consecutive lysines, 4) at least two consecutive arginines, 5) at least two consecutive aspartic acids, 6) at least two consecutive glutamic acids, 7) methionine, 8) tryptophan, and 9) at least five consecutive amino acids comprising no charged amino acids.

28. The method of claim 27 wherein the method further comprises selecting against at least 5 of the characteristics.

29. The method of claim 27 wherein the method further comprises selecting against at least 7 of the characteristics.

30. The method of claim 27 wherein the method further comprises selecting against the 9 characteristics.

31. The method of any one of claims 27-30 wherein the method further comprises:
c) selecting against amino acid sequences of the length and having at least one of the following additional characteristics 1) sequences having at least 5 consecutive amino acids that are identical to an alternative amino acid sequence from an alternative polypeptide that is different from the candidate polypeptide, 2) posttranslational modification sites, and 3) highly hydrophobic sequences.

32. The method of claim 31 wherein the posttranslational modification sites are phosphorylation or glycosylation sites.

33. The method of claim 31 or 32 wherein the method further comprises selecting against at least 2 of the additional characteristics.

34. The method of claim 31 or 32 wherein the method further comprises selecting against the 3 additional characteristics.

35. The method of any one of claims 27-34 wherein the method further comprises performing a BLAST-type or a FAST-type analyses for the candidate polypeptide sequence.

36. The method of any one of claims 27-34 wherein the method further comprises performing a BLAST analysis for the candidate polypeptide sequence.

37. The method of any one of claims 27-36 wherein the antigenic peptide has a length from 6 amino acids to about 50 amino acids.

38. The method of any one of claims 27-36 wherein the antigenic peptide has a length from 6 amino acids to about 20 amino acids.

39. The method of any one of claims 27-36 wherein the antigenic peptide has a length of about 20 amino acids.

40. The method of any one of claims 27-39 wherein the polypeptide is a protein.

41. The method of any one of claims 27-40 wherein the polypeptide is a human protein.

42. The method of any one of claims 27-41 wherein the polypeptide is a naturally occurring protein.

43. An isolated antigenic peptide that is specific for the candidate polypeptide of any one of claims 27-42 that is produced according to the method of any one of claims 27-42.

44. An antigenic peptide that is at least about 90% identical to the isolated antigenic peptide of claim 43.

45. An isolated antigenic peptide that is an analog of the isolated antigenic peptide of claim 43.

46. An isolated antigenic peptide comprising a short antigenic amino acid sequence that is identical to at least 5 consecutive amino acids of the isolated antigenic peptide of claim 43.

47. An isolated antigenic peptide comprising a short antigenic amino acid sequence that is identical to or contains no more than one conservative amino acid substitution over at least 7 consecutive amino acids of the isolated antigenic peptide of claim 43.

48. A kit for the detection of antibodies against the candidate polypeptide of any one of claims 43-47 in a sample comprising:
a) an isolated antigenic peptide according to any one of claims 43-47 and derived from the candidate polypeptide, and
b) at least one of a reagent or a device for detecting the antibodies.

49. An isolated antibody specific for a candidate polypeptide comprising an amino acid sequence that is identical to the amino acid sequence of the isolated antigenic peptide of claim 43, wherein the antibody was produced using the isolated antigenic peptide of claim 43.

50. An isolated antibody specific for a candidate polypeptide comprising an amino acid sequence that is identical to the amino acid sequence of the isolated antigenic peptide of claim 44, wherein the antibody was produced using the isolated antigenic peptide of claim 44.

51. An isolated antibody specific for a candidate polypeptide comprising an amino acid sequence that is identical to the amino acid sequence of the isolated antigenic peptide of claim 45, wherein the antibody was produced using the isolated antigenic peptide of claim 45.

52. An isolated antibody specific for a candidate polypeptide comprising an amino acid sequence that is identical to the amino acid sequence of the isolated antigenic peptide of claim 46, wherein the antibody was produced using the isolated antigenic peptide of claim 46.

53. An isolated antibody specific for a candidate polypeptide comprising an amino acid sequence that is identical to the amino acid sequence of the isolated antigenic peptide of claim 47, wherein the antibody was produced using the isolated antigenic peptide of claim 47.

54. The isolated antibody of any one of claims 49-53 wherein the antibody has high specificity and high affinity for the candidate polypeptide.

55. A kit for the detection of antibodies against the candidate polypeptide of any one of claims 43-47 comprising:
a) an isolated antibody according to any one of claims 49-53, and
b) at least one of a reagent or a device for detecting the antibody.

56. An assay for the detection of a candidate polypeptide in a sample, comprising:
a) providing an isolated antigenic peptide according to any one of claims 43-47,
b) contacting the isolated antigenic peptide with the sample under conditions suitable and for a time sufficient for the antigenic peptide to bind to one or more antibodies specific for the candidate polypeptide present in the sample, to provide an antibody-bound antigenic peptide, and
c) detecting the antibody-bound antigenic peptide, and therefrom determining whether the sample contains the candidate polypeptide.

57. The assay of claim 56 further comprising the step of binding the isolated antigenic peptide or the antibody to a solid substrate.

58. The assay of claim 56 or 57 wherein the sample is an unpurified sample.

59. The assay of any one of claims 56-58 further comprising, prior to the contacting, obtaining the sample from a human being.

60. The assay of any one of claims 56-59 wherein the assay is selected from the group consisting of a countercurrent immuno-electrophoresis (CIEP) assay, a radioimmunoassay, a radioimmunoprecipitation, an enzyme-linked immuno-sorbent assay (ELISA), a dot blot assay, an inhibition or competition assay, a sandwich assay, an immunostick (dip-stick) assays, a simultaneous assay, an immunochromatographic assay, an immunofiltration assay, a latex bead agglutination assay, an immunofluorescent assay, a biosensor assay, and a low-light detection assay.

61. An isolated nucleic acid molecule encoding an antigenic peptide according to any one of claims 43-47.

62. The isolated nucleic acid molecule according to claim 61 wherein the molecule encodes a naturally occurring human antigenic peptide.

63. An isolated nucleic acid molecule encoding an antigenic peptide that is at least about 90% identical to any one of the antigenic peptides set forth in claims 43-47.

64. The isolated nucleic acid molecule according to claim 63 wherein the antigenic peptide is at least about 95% identical to the antigenic peptide.

65. The isolated nucleic acid molecule according to claim 63 or 64 wherein the molecule encodes a naturally occurring human antigenic peptide.

66. A process for producing an isolated polynucleotide comprising hybridizing a nucleotide encoding an antigenic peptide according to any one of claims 43-47 to genomic DNA under highly stringent conditions and isolating the polynucleotide detected with the nucleotide.
